(19) United States
(12) Patent Application Publication
(10) Pub. No.: US 2024/0344146 A1
(43) Pub. Date: Oct. 17, 2024

(54) KIT, DEVICE, AND METHOD FOR DETECTING LUNG CANCER

(71) Applicants: TORAY INDUSTRIES, INC., Tokyo (JP); NATIONAL CANCER CENTER, Tokyo (JP)

(72) Inventors: Yuho KIDA, Kamakura-shi (JP); Satoko KOZONO, Kamakura-shi (JP); Hiroko SUDO, Kamakura-shi (JP); Satoshi KONDOU, Kamakura-shi (JP); Junpei KAWAUCHI, Kamakura-shi (JP); Takahiro OCHIYA, Tokyo (JP); Shun-ichi WATANABE, Tokyo (JP)

(73) Assignees: TORAY INDUSTRIES, INC., Tokyo (JP); NATIONAL CANCER CENTER, Tokyo (JP)

(21) Appl. No.: 18/739,680

(22) Filed: Jun. 11, 2024

Related U.S. Application Data
(62) Division of application No. 18/120,059, filed on Mar. 10, 2023, now Pat. No. 12,043,872.
Application No. 18/120,059 is a division of application No. 16/626,781, filed on Dec. 26, 2019, now Pat. No. 11,634,778.

(30) Foreign Application Priority Data
Jun. 29, 2017 (JP) 2017-126933

(51) Int. Cl. C12Q 1/6886; C12Q 1/6816

(52) U.S. Cl. CPC C12Q 1/6886; C12Q 1/6816; C12Q 2600/112; C12Q 2600/178; G01N 2800/7028

(57) ABSTRACT

This application provides a kit or a device for detection of lung cancer, comprising a nucleic acid(s) for detecting a miRNA(s) in a sample from a subject, and a method for detecting lung cancer, comprising measuring the miRNA(s) in vitro.

### CROSS-REFERENCE TO RELATED APPLICATIONS

This application is a Divisional of copending U.S. application Ser. No. 18/120,059 filed on Mar. 10, 2023, which is a Divisional of copending application Ser. No. 16/626,781, filed on Dec. 26, 2019 (now U.S. Pat. No. 11,634,778 issued Apr. 25, 2023), which is the National Phase under 35 U.S.C. § 371 of International Application No. PCT/JP2018/024834, filed on Jun. 29, 2018, which claims the benefit under 35 U.S.C. § 119(a) to Patent Application No. 2017-126933, filed in Japan on Jun. 29, 2017, all of which are hereby expressly incorporated by reference into the present application.

### REFERENCE TO ELECTRONIC SEQUENCE LISTING

This application contains a Sequence Listing which has been submitted electronically in .XML format and is hereby incorporated by reference in its entirety. Said .XML copy, created on Feb. 28, 2023, is named “PH-7402-PCT_Sequence Listing” and is 277,496 bytes in size. The sequence listing contained in this .XML file is part of the specification and is hereby incorporated by reference herein in its entirety.

### TECHNICAL FIELD

The present invention relates to a kit or a device for detection of lung cancer, comprising a nucleic acid capable of specifically binding to a particular polynucleotide or a polynucleotide complementary to that of the polynucleotide, which is used for examining the presence or absence of lung cancer in a subject, and a method for detecting lung cancer by measuring an expression level of the miRNA using the nucleic acid.

### BACKGROUND ART

Lung cancer is a cancerous change of some cells of the trachea, bronchi, or alveoli of the lungs for some reasons. According to the cancer statistics in the year of 2012 by site in Japan disclosed by the Center for Cancer Control and Information Services, National Cancer Center, the number of people affected by lung cancer is 107,241 people, and 1 out of every 10 men and 1 out of every 22 women are supposedly affected. The number of deaths from lung cancer in men and women all together climbed to 71,518 people which is the top cause of deaths among cancer types. In the United States, the estimated number of people affected by lung cancer in 2014 climbed to 224,210 people, out of which about 159,260 people are expected to die.

Lung cancer has several different tissue types, of which about 15% is small cell lung carcinoma, whereas the remaining tissue types are called non-small cell lung carcinoma. Non-small cell lung carcinoma contains various tissue types, including three major tissue types that are adenocarcinoma, squamous cell carcinoma, and large cell carcinoma. Depending on the tissue type of lung cancer, development site, form and speed of progression, and their symptoms notably vary, and appropriate approaches to therapy are accordingly different. For example, small cell carcinoma quickly grows and is highly malignant, but is said to be more susceptible to anticancer agents and radiation treatment than non-small cell carcinoma. Furthermore, classification by the development site of lung cancer primarily includes hilar type and lung field type. The hilar type which develops near the hilar area at which various tracheas come together is not easily detected by a typical X-ray examination compared to the lung field type which develops in the periphery of the lung.

A stage of progression of lung cancer is classified into stages 0, IA, IB, IIA, IIB, IIIA, IIIB, and IV according to spread of tumors (T1 to T4), lymph node metastasis (NO to N3), and distant metastasis (M0, M1). Survival rate in lung cancer varies depending on the stage of progression. Five-year relative survival rates in non-small cell lung carcinoma are reported to be 45 to 49% in the case of stage I (IA and IB), 30 to 31% in the case of stage II (IIA and IIB), 5 to 14% in the case of stage III (IIIA and IIIB), and 1% in the case of stage IV. Thus, detection of lung cancer in an early stage, i.e., detection in stage 0 or stage I, and treatment thereof notably contribute to improvement in survival rate.

Lung cancer is mainly treated by surgery, radiation therapy, and anticancer agents. Particularly, surgery is suitable for an early stage lung cancer, and it is likely to be cured in such case. Furthermore, in the case of an early stage lung cancer, there are several treatment selections that are less burden on a patient are available; such treatment includes thoracoscopic surgery, stereotactic body radiation treatment (SBRT), photodynamic treatment, laser treatment, brachytherapy for irradiating radiation from inside the body, or the like.

Many lung cancer cases are, regardless of a stage of progression, almost asymptomatic, which makes early detection at routine health checkups important. The most common lung cancer screening is chest X-ray examination. When a suspecting result is obtained from chest X-ray examination, a more precise image diagnoses such as CT test, MRI test, PET test, or the like, are carried out. Additionally, in recent years effectiveness of low dose CT on lung cancer screening has been recognized. National Lung Screening Trial conducted in the United States revealed that a test subject group of high-risk for lung cancer such as chain smokers who took CT checkups had reduced mortalities compared to those who took chest X-ray checkups.

When an individual is strongly suspected of having lung cancer by an image diagnosis, final diagnosis would be made that includes determination of the lung cancer tissue type by collecting cells and tissues for a microscopic examination. Cytodiagnosis and tissue diagnosis include sputum cytodiagnosis, pleural effusion examination, bronchoscopy, percutaneous needle biopsy, and the like.

In conventional diagnostic methods, lung cancers are often found in a progressed state, and they were found by the methods that impose an ineligible burden on the examinee, such as biopsy test. Under the circumstance, there is an effort to detect lung cancers earlier in a simpler manner using tumor markers in blood. Examples of the lung cancer tumor markers used at present include CEA, CYFRA21-1, NSE, SCC, and the like. As shown in Patent Literatures 1 to 6 and Non-Patent Literatures 1 to 6, there are reports, albeit at a research stage, on the detection of lung cancer using the expression levels of microRNAs (miRNAs) in biological samples including blood.
Specifically, Patent Literature 1 discloses a method for discriminating lung cancer patients from healthy subjects and patients with cancers other than lung cancer using miR-1343-3p, miR-6746-5p, miR-187-5p, miR-4632-5p and the like in serum.
Patent Literature 2 discloses a method for discriminating lung cancer using a pair of expression levels of miR-296-5p, miR-422a, miR-638, miR-191-5p, miR-23a-3p, miR-24-3p, miR-320a, miR-29b-3p, miR-92-3p and the like in serum or plasma.
Patent Literature 3 discloses miR-150-3p, miR-103a-3p, miR-107 and the like in peripheral blood as biomarkers for non-small cell lung carcinoma.
Patent Literature 4 discloses a method for discriminating non-small cell lung carcinoma using miR-23b-3p, miR-29b-3p, miR-625-3p, miR-17-3p and the like in peripheral blood.
Patent Literature 5 discloses a method for diagnosing lung cancer or predicting prognosis for lung cancer using miR-1249-3p, miR-1275, miR-191-5p, miR-423-5p, miR-744, miR-874-3p and the like in tracheal cells.
Patent Literature 6 discloses a method for detecting lung cancer patients from high-risk people for lung cancer with high smoking amount using miR-23b-3p, miR-107, miR-103a-3p, miR-17-5p and the like in plasma.
Non-Patent Literature 1 discloses a method for discriminating lung adenocarcinoma from healthy subjects and lung benign diseases using twenty miRNAs including miR-1290 and miR-24-3p in serum.
Non-Patent Literature 2 discloses that expression levels of five miRNAs including miR-650 in bronchoalveolar lavage samples significantly increase in lung cancer patients whereby these miRNAs have potential for the use as lung cancer markers.
Non-Patent Literature 3 discloses a method for discriminating lung cancer patients from healthy subjects using expression levels of miR-3180-3p, miR-342-5p, miR-150 and the like contained in neutrophilic granulocyte.
Non-Patent Literature 4 discloses miR-550 and the like in serum as biomarkers for lung adenocarcinoma.
Non-Patent Literature 5 discloses miR-1229 and the like in serum as biomarkers for non-small cell lung carcinoma.
Non-Patent Literature 6 discloses miR-1254, miR-1275, miR-320a and the like in serum as biomarkers for non-small cell lung carcinoma.

### PRIOR ART LITERATURE

### Patent Literature

Patent Literature 1: International Publication No. WO 2015/194610
Patent Literature 2: JP Patent Publication (Kohyo) No. 2013-502931 A (2013)
Patent Literature 3: JP Patent Publication (Kohyo) No. 2011-505143 A (2011)
Patent Literature 4: Published U.S. Patent Application No. 2012/108462
Patent Literature 5: Published U.S. Patent Application No. 2015/080243
Patent Literature 6: International Publication No. WO 2015/115923

### Non-Patent Literature

Non-Patent Literature 1: Tai M C et al. Sci Rep. 2016 Aug. 10; 6: 31389
Non-Patent Literature 2: Schmidt B et al. Adv Exp Med Biol. 2016; 924: 33-37.
Non-Patent Literature 3: Leidinger P et al. Oncotarget. 2014 Oct. 15; 5(19): 9484-97.
Non-Patent Literature 4: Rani S et al. Cancer Biol Ther. 2013 December; 14(12): 1104-12.
Non-Patent Literature 5: Roth C et al. PLoS One. 2012; 7(6): e38248
Non-Patent Literature 6: Foss K M et al. J Thorac Oncol. 2011 March; 6(3): 482-8

### SUMMARY OF INVENTION

### Problem to be Solved by Invention

An object of the present invention is to find a novel tumor marker(s) for lung cancer practically usable in primary tests of lung cancer and to provide a method that can effectively detect lung cancer using a nucleic acid(s) that specifically bind(s) to the marker(s). Effective primary tests of lung cancer require four factors: 1. ability to detect early stages, 2. Ability to detect any histological type of lung cancer, 3. high detection sensitivity and specificity for lung cancer, and 4. low invasiveness to examinees. An object of the present invention is to provide a test method that satisfies these factors.

X-ray examination, which is currently used as a main primary test of lung cancer, has the difficulty in the early detection of small cell carcinoma or squamous cell carcinoma which develops mainly in the hilar area. Large cell carcinoma grows rapidly and often already has a large tumor size when detected. Furthermore, some who are detected as abnormal in low-dose CT examination often turn out to be non-cancer (false-positive) by additional examination. Such case could lead to more highly invasive needle biopsy or surgery if unattended.

For detection of lung cancer, CEA and CYFRA21-1 are known examples as tumor markers in blood. These tumor markers in blood, however, have been reported to have general lung cancer detection sensitivity of 69% (CEA) and 43% (CYFRA21-1), and are thus not very useful in lung cancer examination. Furthermore, the tumor markers such as CEA and CYFRA21-1 may elevate for reasons other than lung cancer, and therefore have the difficulty in identifying cancer types. The false diagnosis of other cancers as lung cancer wastes appropriate therapeutic opportunity or places unnecessary economical and physical burdens on patients due to the application of wrong medical approaches.

As described below, there are reports, albeit at a research stage, on the determination of lung cancer using the expression levels of microRNAs (miRNAs) in biological samples including blood, none of which, however, have yet been brought into practical use.
Patent Literature 1 discloses a method for discriminating a lung cancer patient from a healthy subject or a patient having a cancer other than lung cancer using miR-1343-3p, miR-6746-5p, miR-187-5p, miR-4632-5p and the like in serum. However, lung cancer sample groups include only adenocarcinoma and squamous cell carcinoma samples. Therefore, this method might be unable to detect the other types of lung cancers.
Patent Literature 2 discloses a method for determining lung cancer using a pair of expression levels of miR-296-5p, miR-422a, miR-638, miR-191-5p, miR-23a-3p, miR-24-3p, miR-320a, miR-29b-3p, miR-92-3p and the like in serum or plasma. However, any cancer sample other than lung cancer was not used in the Examples. Therefore, the method might misdiagnose lung cancer as another cancer.
Patent Literature 3 discloses miR-150-3p, miR-103a-3p, miR-107 and the like in peripheral blood as biomarkers for non-small cell lung carcinoma; however, it does not describe the specific detection performance, such as accuracy, sensitivity, or specificity, for determining lung cancer, making these miRNAs poor in industrially practical use.
Patent Literature 4 discloses a method for determining non-small cell lung carcinoma using miR-23b-3p, miR-29b-3p, miR-625-3p, miR-17-3p and the like in peripheral blood. However, any cancer sample other than lung cancer, or any other cancer type other than small cell carcinoma was not used in the Examples. Therefore, the method might misdiagnose lung cancer as another cancer, or might be unable to detect some types of lung cancers such as small cell carcinoma.
Patent Literature 5 discloses a method for diagnosing lung cancer or predicting prognosis for lung cancer using miR-1249-3p, miR-1275, miR-191-5p, miR-423-5p, miR-744, miR-874-3p and the like in tracheal cells. However, obtaining tissue samples requires tissue resection by surgery, and this step causes an undue physical burden on a patient, hence not preferable as a test method.
Patent Literature 6 discloses a method for detecting a lung cancer patient from people who have a large quantity of smoking and have a high risk of lung cancer using miR-23b-3p, miR-107, miR-103a-3p, miR-17-5p and the like in plasma. However, any cancer sample other than lung cancer was not used in the Examples. Therefore, the method might misdiagnose lung cancer as another cancer.
Non-Patent Literature 1 discloses a method for discriminating lung adenocarcinoma from normal health or benign lung disease using twenty miRNAs including miR-1290 and miR-24-3p in serum. However, the discriminant performance for squamous cell carcinoma or small cell carcinoma is as low as approximately 70%. Therefore, the method might overlook some histological types of lung cancer patients.
Non-Patent Literature 2 discloses that expression levels of five miRNAs including miR-650 in bronchoalveolar lavage samples were significantly increased in lung cancer patients, indicating their potentiality as lung cancer markers; however, it does not describe the specific detection performance, such as accuracy, sensitivity, or specificity, for determining lung cancer, making these miRNAs poor in industrially practical use.
Non-Patent Literature 3 discloses a method for discriminating a lung cancer patient from a healthy subject using expression levels of miR-3180-3p, miR-342-5p, miR-150 and the like contained in neutrophilic granulocytes. However, separation of immunocytes on a cell type basis from blood is laborious, making these miRNAs poor in industrially practical use.
Non-Patent Literature 4 discloses miR-550 and the like in serum as biomarkers for lung adenocarcinoma. However, any cancer sample other than lung cancer, or any lung cancer type other than adenocarcinoma was not used in the Examples. Therefore, use of these miRNAs as biomarkers might misdiagnose lung cancer as another cancer or might be unable to detect some histological types of lung cancers.
Non-Patent Literature 5 discloses miR-1229 and the like in serum as biomarkers for non-small cell lung carcinoma. However, any cancer sample other than lung cancer, or any small cell carcinoma sample was not used in the Examples. Therefore, use of these miRNAs as the biomarker might misdiagnose lung cancer as another cancer or might be unable to detect some histological types of lung cancers.
Non-Patent Literature 6 discloses miR-1254, miR-1275, miR-320a and the like in serum as biomarkers for non-small cell lung carcinoma. However, any cancer sample other than lung cancer, or any small cell carcinoma sample was not used in the Examples. Therefore, use of these miRNAs as the biomarker might misdiagnose lung cancer as another cancer, or might be unable to detect some types of lung cancers such as small cell carcinoma.

As mentioned above, chest X-ray examination or low-dose CT for use in lung cancer examination has the difficulty in detection of lung cancer, depending on the site of origin, and in some case detect non-cancer abnormality in an image that might lead to the execution of needless extra examination. Furthermore, the existing tumor markers exhibit low detection performance for lung cancer and cannot distinguish lung cancer from other cancers. Neither validation using samples of cancers other than lung cancer nor validation using some histological types of lung cancers such as small cell carcinoma or large cell carcinoma has been conducted as to the markers at a research stage. Therefore, use of these markers might require carrying out needless extra examination due to the false detection of normal subjects or other cancer patients as being lung cancer patients, or might waste therapeutic opportunity because of overlooking lung cancer patients. Furthermore, the collection of lung tissues for measuring the tumor markers is highly invasive to patients and is not favorable. Hence, there is a demand for a highly accurate lung cancer marker that is detectable from blood, which can be collected with less invasiveness, and is capable of correctly determining the presence or absence of lung cancer. Particularly, the early detection of lung cancer increases the applicability of surgery in treatment, and drastically improve the survival rates. For early-stage lung cancers, there are several therapeutic options available that place less burden on patients, such as thoracoscopic surgery and stereotactic body radiotherapy. Therefore, a highly sensitive lung cancer marker that can detect lung cancer even at a low stage of progression is desired.

### Means for Solution of Problem

The present inventors have conducted diligent studies to attain the object and consequently completed the present invention by finding gene markers usable as markers for detection of lung cancer from blood, which can be collected with limited invasiveness, and finding that lung cancer such as lung adenocarcinoma, lung squamous cell carcinoma, large cell lung carcinoma, or small cell lung carcinoma can be significantly, preferably specifically, detected, using nucleic acids to detect such markers, for example, at least one nucleic acid selected from probes capable of specifically binding to any of these markers and primers for amplifying these markers.

### SUMMARY OF INVENTION

The present invention has the following features:
(1) A kit for detection of lung cancer, comprising a nucleic acid(s) capable of specifically binding to at least one polynucleotide selected from the group consisting of the following lung cancer markers: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p, or to a polynucleotide consisting of a nucleotide sequence complementary to that of the polynucleotide.
(2) The kit according to (1), wherein the nucleic acid(s) is a polynucleotide(s) selected from the group consisting of the following polynucleotides (a) to (e):
(a) a polynucleotide consisting of a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(b) a polynucleotide comprising a nucleotide sequence represented by any of SEQ ID NOs: 1 to 163;
(c) a polynucleotide consisting of a nucleotide sequence complementary to a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 163 or a nucleotide sequence derived from the nucleotide sequence in which the nucleic acid u is replaced with t; and
(e) a polynucleotide hybridizing under stringent conditions to any of the polynucleotides (a) to (d).
(3) The kit according to (1) or (2), wherein the kit further comprises a nucleic acid(s) capable of specifically binding to at least one polynucleotide selected from the group consisting of the following other lung cancer markers: miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, or to a polynucleotide consisting of a nucleotide sequence complementary to that of the polynucleotide.
(4) The kit according to (3), wherein the nucleic acid(s) is a polynucleotide(s) selected from the group consisting of the following polynucleotides (f) to (j):
(f) a polynucleotide consisting of a nucleotide sequence represented by any of SEQ ID NOs: 164 to 329 or a nucleotide sequence derived from the nucleotide sequence in which the nucleic acid u is replaced with t, a variant thereof, a derivative thereof, or a fragment thereof comprising 15 or more consecutive nucleotides;
(g) a polynucleotide comprising a nucleotide sequence represented by any of SEQ ID NOs: 164 to 329;
(h) a polynucleotide consisting of a nucleotide sequence complementary to a nucleotide sequence represented by any of SEQ ID NOs: 164 to 329 or a nucleotide sequence derived from the nucleotide sequence in which the nucleic acid u is replaced with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 164 to 329 or a nucleotide sequence derived from the nucleotide sequence in which the nucleic acid u is replaced with t; and
(j) a polynucleotide hybridizing under stringent conditions to any of the polynucleotides (f) to (i).
(5) A device for detection of lung cancer, comprising a nucleic acid(s) capable of specifically binding to at least one polynucleotide selected from the group consisting of the following lung cancer markers: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p, or to a polynucleotide consisting of a nucleotide sequence complementary to that of the polynucleotide.
(6) The device according to (5), wherein the nucleic acid(s) is a polynucleotide(s) selected from the group consisting of the following polynucleotides (a) to (e):
(a) a polynucleotide consisting of a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(b) a polynucleotide comprising a nucleotide sequence represented by any of SEQ ID NOs: 1 to 163;
(c) a polynucleotide consisting of a nucleotide sequence complementary to a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 163 or a nucleotide sequence derived from the nucleotide sequence in which the nucleic acid u is replaced with t; and
(e) a polynucleotide hybridizing under stringent conditions to any of the polynucleotides (a) to (d).
(7) The device according to (5) or (6), wherein the device further comprises a nucleic acid(s) capable of specifically binding to at least one polynucleotide selected from the group consisting of the following other lung cancer markers: miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, or to a polynucleotide consisting of a nucleotide sequence complementary to that of the polynucleotide.
(8) The device according to (7), wherein the nucleic acid(s) is a polynucleotide(s) selected from the group consisting of the following polynucleotides (f) to (j):
(f) a polynucleotide consisting of a nucleotide sequence represented by any of SEQ ID NOs: 164 to 329 or a nucleotide sequence derived from the nucleotide sequence in which the nucleic acid u is replaced with t, a variant thereof, a derivative thereof, or a fragment thereof comprising 15 or more consecutive nucleotides;
(g) a polynucleotide comprising a nucleotide sequence represented by any of SEQ ID NOs: 164 to 329;
(h) a polynucleotide consisting of a nucleotide sequence complementary to a nucleotide sequence represented by any of SEQ ID NOs: 164 to 329 or a nucleotide sequence derived from the nucleotide sequence in which the nucleic acid u is replaced with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 164 to 329 or a nucleotide sequence derived from the nucleotide sequence in which the nucleic acid u is replaced with t; and
(j) a polynucleotide hybridizing under stringent conditions to any of the polynucleotides (f) to (i).
(9) The device according to any of (5) to (8), wherein the device is for measurement by a hybridization technique.
(10) The device according to (9), wherein the hybridization technique is a nucleic acid array technique.
(11) A method for detecting lung cancer, comprising: measuring an expression level(s) of at least one polynucleotide selected from the group consisting of the following lung cancer markers: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p in a sample from a subject; and evaluating in vitro whether or not the subject has lung cancer using the measured expression level(s).
(12) A method for detecting lung cancer, comprising: measuring an expression level(s) of at least one polynucleotide selected from the group consisting of the following lung cancer markers: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p in a sample from a subject; and evaluating in vitro whether or not the subject has lung cancer using both of the measured expression level(s) and a control expression level(s) from healthy subjects measured in the same way.
(13) A method for detecting lung cancer, comprising: measuring an expression level(s) of at least one polynucleotide selected from the group consisting of the following lung cancer markers: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p in a sample from a subject; and assigning the expression level(s) of the at least one polynucleotide in the sample from the subject to a discriminant, which is capable of discriminatorily determining the presence or absence of lung cancer, and is prepared with gene expression levels in samples from subjects known to have lung cancer and samples from subjects without lung cancer as training samples, and thereby evaluating in vitro the presence or absence of lung cancer.
(14) The method according to any of (11) to (13), wherein the measurement of the expression level(s) of the polynucleotide(s) is performed using a nucleic acid(s) capable of specifically binding to the polynucleotide(s) or a polynucleotide(s) consisting of a nucleotide sequence(s) complementary to the polynucleotide(s), wherein the nucleic acid(s) is a polynucleotide(s) selected from the group consisting of the following polynucleotides (a) to (e):
(a) a polynucleotide consisting of a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(b) a polynucleotide comprising a nucleotide sequence represented by any of SEQ ID NOs: 1 to 163;
(c) a polynucleotide consisting of a nucleotide sequence complementary to a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 163 or a nucleotide sequence derived from the nucleotide sequence in which the nucleic acid u is replaced with t; and
(e) a polynucleotide hybridizing under stringent conditions to any of the polynucleotides (a) to (d).
(15) The method according to any of (11) to (14), wherein the method further comprises measuring an expression level(s) of at least one polynucleotide selected from the group consisting of the following other lung cancer markers: miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p.
(16) The method according to (15), wherein the measurement of the expression level(s) of the polynucleotide(s) is performed using a nucleic acid(s) capable of specifically binding to the polynucleotide(s) or a polynucleotide(s) consisting of a nucleotide sequence(s) complementary to the polynucleotide(s), wherein the nucleic acid(s) is a polynucleotide(s) selected from the group consisting of the following polynucleotides (f) to (j):
(f) a polynucleotide consisting of a nucleotide sequence represented by any of SEQ ID NOs: 164 to 329 or a nucleotide sequence derived from the nucleotide sequence in which the nucleic acid u is replaced with t, a variant thereof, a derivative thereof, or a fragment thereof comprising 15 or more consecutive nucleotides;
(g) a polynucleotide comprising a nucleotide sequence represented by any of SEQ ID NOs: 164 to 329;
(h) a polynucleotide consisting of a nucleotide sequence complementary to a nucleotide sequence represented by any of SEQ ID NOs: 164 to 329 or a nucleotide sequence derived from the nucleotide sequence in which the nucleic acid u is replaced with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 164 to 329 or a nucleotide sequence derived from the nucleotide sequence in which the nucleic acid u is replaced with t; and
(j) a polynucleotide hybridizing under stringent conditions to any of the polynucleotides (f) to (i).
(17) The method according to any of (11) to (16), wherein the expression level(s) of the polynucleotide(s) in the sample from the subject is measured using a kit according to any of (1) to (4) or a device according to any of (5) to (10), comprising a nucleic acid(s) capable of specifically binding to the polynucleotide(s) or a polynucleotide(s) consisting of a nucleotide sequence(s) complementary to the polynucleotide(s).
(18) The method according to any of (11) to (17), wherein the subject is a human.
(19) The method according to any of (11) to (18), wherein the sample is blood, serum, or plasma.

In one preferred aspect of the present invention, the kit, the device or the method for detecting lung cancer according to the present invention is a kit, a device or a method for detecting lung cancer, wherein a histological type of the lung cancer is adenocarcinoma, squamous cell carcinoma, large cell carcinoma or small cell carcinoma.

In another preferred aspect of the present invention, the kit, the device or the method for detecting lung cancer according to the present invention is a kit, a device or a method for detecting lung cancer, wherein as the lung cancer markers, miR-6787-5p is hsa-miR-6787-5p, miR-920 is hsa-miR-920, miR-3622a-5p is hsa-miR-3622a-5p, miR-1185-1-3p is hsa-miR-1185-1-3p, miR-4327 is hsa-miR-4327, miR-5739 is hsa-miR-5739, miR-937-5p is hsa-miR-937-5p, miR-1181 is hsa-miR-1181, miR-1185-2-3p is hsa-miR-1185-2-3p, miR-1193 is hsa-miR-1193, miR-1207-5p is hsa-miR-1207-5p, miR-1238-5p is hsa-miR-1238-5p, miR-1246 is hsa-miR-1246, miR-1249-5p is hsa-miR-1249-5p, miR-1292-3p is hsa-miR-1292-3p, miR-1469 is hsa-miR-1469, miR-1470 is hsa-miR-1470, miR-197-5p is hsa-miR-197-5p, miR-208a-5p is hsa-miR-208a-5p, miR-2110 is hsa-miR-2110, miR-211-3p is hsa-miR-211-3p, miR-2467-3p is hsa-miR-2467-3p, miR-3122 is hsa-miR-3122, miR-3141 is hsa-miR-3141, miR-3156-5p is hsa-miR-3156-5p, miR-3158-5p is hsa-miR-3158-5p, miR-3160-5p is hsa-miR-3160-5p, miR-3180-3p is hsa-miR-3180-3p, miR-3191-3p is hsa-miR-3191-3p, miR-3194-3p is hsa-miR-3194-3p, miR-320b is hsa-miR-320b, miR-328-5p is hsa-miR-328-5p, miR-3610 is hsa-miR-3610, miR-3619-3p is hsa-miR-3619-3p, miR-3620-5p is hsa-miR-3620-5p, miR-370-3p is hsa-miR-370-3p, miR-373-5p is hsa-miR-373-5p, miR-3917 is hsa-miR-3917, miR-3937 is hsa-miR-3937, miR-4259 is hsa-miR-4259, miR-4281 is hsa-miR-4281, miR-4294 is hsa-miR-4294, miR-4419b is hsa-miR-4419b, miR-4428 is hsa-miR-4428, miR-4429 is hsa-miR-4429, miR-4433a-3p is hsa-miR-4433a-3p, miR-4447 is hsa-miR-4447, miR-4449 is hsa-miR-4449, miR-4459 is hsa-miR-4459, miR-4480 is hsa-miR-4480, miR-4485-5p is hsa-miR-4485-5p, miR-4486 is hsa-miR-4486, miR-4488 is hsa-miR-4488, miR-4489 is hsa-miR-4489, miR-4505 is hsa-miR-4505, miR-4513 is hsa-miR-4513, miR-4515 is hsa-miR-4515, miR-4530 is hsa-miR-4530, miR-4535 is hsa-miR-4535, miR-4635 is hsa-miR-4635, miR-4640-5p is hsa-miR-4640-5p, miR-4646-5p is hsa-miR-4646-5p, miR-4656 is hsa-miR-4656, miR-4663 is hsa-miR-4663, miR-4665-5p is hsa-miR-4665-5p, miR-4706 is hsa-miR-4706, miR-4707-5p is hsa-miR-4707-5p, miR-4708-3p is hsa-miR-4708-3p, miR-4710 is hsa-miR-4710, miR-4718 is hsa-miR-4718, miR-4722-5p is hsa-miR-4722-5p, miR-4727-3p is hsa-miR-4727-3p, miR-4730 is hsa-miR-4730, miR-4734 is hsa-miR-4734, miR-4740-5p is hsa-miR-4740-5p, miR-4747-3p is hsa-miR-4747-3p, miR-4749-5p is hsa-miR-4749-5p, miR-4755-3p is hsa-miR-4755-3p, miR-4763-5p is hsa-miR-4763-5p, miR-4787-3p is hsa-miR-4787-3p, miR-5008-5p is hsa-miR-5008-5p, miR-5010-5p is hsa-miR-5010-5p, miR-504-3p is hsa-miR-504-3p, miR-5090 is hsa-miR-5090, miR-5100 is hsa-miR-5100, miR-5196-5p is hsa-miR-5196-5p, miR-551b-5p is hsa-miR-551b-5p, miR-557 is hsa-miR-557, miR-5787 is hsa-miR-5787, miR-6090 is hsa-miR-6090, miR-6124 is hsa-miR-6124, miR-6132 is hsa-miR-6132, miR-6510-5p is hsa-miR-6510-5p, miR-6511b-5p is hsa-miR-6511b-5p, miR-6515-3p is hsa-miR-6515-3p, miR-654-5p is hsa-miR-654-5p, miR-658 is hsa-miR-658, miR-668-5p is hsa-miR-668-5p, miR-6722-5p is hsa-miR-6722-5p, miR-6724-5p is hsa-miR-6724-5p, miR-6729-3p is hsa-miR-6729-3p, miR-6737-5p is hsa-miR-6737-5p, miR-6756-5p is hsa-miR-6756-5p, miR-6762-5p is hsa-miR-6762-5p, miR-6763-3p is hsa-miR-6763-3p, miR-6766-5p is hsa-miR-6766-5p, miR-6769a-5p is hsa-miR-6769a-5p, miR-6771-5p is hsa-miR-6771-5p, miR-6786-5p is hsa-miR-6786-5p, miR-6789-5p is hsa-miR-6789-5p, miR-6794-5p is hsa-miR-6794-5p, miR-6796-3p is hsa-miR-6796-3p, miR-6797-5p is hsa-miR-6797-5p, miR-6800-3p is hsa-miR-6800-3p, miR-6802-5p is hsa-miR-6802-5p, miR-6803-5p is hsa-miR-6803-5p, miR-6805-3p is hsa-miR-6805-3p, miR-6805-5p is hsa-miR-6805-5p, miR-6807-5p is hsa-miR-6807-5p, miR-6812-5p is hsa-miR-6812-5p, miR-6819-5p is hsa-miR-6819-5p, miR-6822-5p is hsa-miR-6822-5p, miR-6824-5p is hsa-miR-6824-5p, miR-6826-5p is hsa-miR-6826-5p, miR-6850-5p is hsa-miR-6850-5p, miR-6858-5p is hsa-miR-6858-5p, miR-6861-5p is hsa-miR-6861-5p, miR-6880-3p is hsa-miR-6880-3p, miR-7107-5p is hsa-miR-7107-5p, miR-7109-5p is hsa-miR-7109-5p, miR-7114-5p is hsa-miR-7114-5p, miR-7704 is hsa-miR-7704, miR-7846-3p is hsa-miR-7846-3p, miR-8052 is hsa-miR-8052, miR-8060 is hsa-miR-8060, miR-8071 is hsa-miR-8071, miR-8073 is hsa-miR-8073, miR-874-5p is hsa-miR-874-5p, miR-204-3p is hsa-miR-204-3p, miR-3154 is hsa-miR-3154, miR-3960 is hsa-miR-3960, miR-4433a-5p is hsa-miR-4433a-5p, miR-4455 is hsa-miR-4455, miR-4462 is hsa-miR-4462, miR-4476 is hsa-miR-4476, miR-4508 is hsa-miR-4508, miR-4687-3p is hsa-miR-4687-3p, miR-4687-5p is hsa-miR-4687-5p, miR-4732-5p is hsa-miR-4732-5p, miR-4771 is hsa-miR-4771, miR-642a-3p is hsa-miR-642a-3p, miR-6732-5p is hsa-miR-6732-5p, miR-6760-5p is hsa-miR-6760-5p, miR-6799-5p is hsa-miR-6799-5p, miR-6820-5p is hsa-miR-6820-5p, miR-6821-5p is hsa-miR-6821-5p, miR-6829-5p is hsa-miR-6829-5p, miR-6893-5p is hsa-miR-6893-5p, miR-7108-3p is hsa-miR-7108-3p, miR-7111-5p is hsa-miR-7111-5p, miR-8089 is hsa-miR-8089, miR-885-3p is hsa-miR-885-3p, miR-92b-3p is hsa-miR-92b-3p, miR-1343-3p is hsa-miR-1343-3p, miR-6746-5p is hsa-miR-6746-5p, miR-422a is hsa-miR-422a, miR-187-5p is hsa-miR-187-5p, miR-4632-5p is hsa-miR-4632-5p, miR-6791-5p is hsa-miR-6791-5p, miR-103a-3p is hsa-miR-103a-3p, miR-107 is hsa-miR-107, miR-1199-5p is hsa-miR-1199-5p, miR-1225-3p is hsa-miR-1225-3p, miR-1225-5p is hsa-miR-1225-5p, miR-1228-5p is hsa-miR-1228-5p, miR-1229-5p is hsa-miR-1229-5p, miR-1233-5p is hsa-miR-1233-5p, miR-1237-5p is hsa-miR-1237-5p, miR-1247-3p is hsa-miR-1247-3p, miR-1249-3p is hsa-miR-1249-3p, miR-1254 is hsa-miR-1254, miR-1260b is hsa-miR-1260b, miR-1268a is hsa-miR-1268a, miR-1268b is hsa-miR-1268b, miR-1273g-3p is hsa-miR-1273g-3p, miR-128-1-5p is hsa-miR-128-1-5p, miR-128-2-5p is hsa-miR-128-2-5p, miR-1290 is hsa-miR-1290, miR-150-3p is hsa-miR-150-3p, miR-17-3p is hsa-miR-17-3p, miR-1908-5p is hsa-miR-1908-5p, miR-1909-3p is hsa-miR-1909-3p, miR-1914-3p is hsa-miR-1914-3p, miR-1915-3p is hsa-miR-1915-3p, miR-191-5p is hsa-miR-191-5p, miR-22-3p is hsa-miR-22-3p, miR-23b-3p is hsa-miR-23b-3p, miR-24-3p is hsa-miR-24-3p, miR-296-3p is hsa-miR-296-3p, miR-296-5p is hsa-miR-296-5p, miR-3131 is hsa-miR-3131, miR-3162-5p is hsa-miR-3162-5p, miR-3188 is hsa-miR-3188, miR-3196 is hsa-miR-3196, miR-3197 is hsa-miR-3197, miR-320a is hsa-miR-320a, miR-342-5p is hsa-miR-342-5p, miR-3621 is hsa-miR-3621, miR-3648 is hsa-miR-3648, miR-3656 is hsa-miR-3656, miR-365a-5p is hsa-miR-365a-5p, miR-3665 is hsa-miR-3665, miR-3679-5p is hsa-miR-3679-5p, miR-371a-5p is hsa-miR-371a-5p, miR-3940-5p is hsa-miR-3940-5p, miR-423-5p is hsa-miR-423-5p, miR-4257 is hsa-miR-4257, miR-4270 is hsa-miR-4270, miR-4271 is hsa-miR-4271, miR-4286 is hsa-miR-4286, miR-4298 is hsa-miR-4298, miR-4417 is hsa-miR-4417, miR-4442 is hsa-miR-4442, miR-4446-3p is hsa-miR-4446-3p, miR-4448 is hsa-miR-4448, miR-4454 is hsa-miR-4454, miR-4467 is hsa-miR-4467, miR-4472 is hsa-miR-4472, miR-4507 is hsa-miR-4507, miR-4516 is hsa-miR-4516, miR-451a is hsa-miR-451a, miR-4649-5p is hsa-miR-4649-5p, miR-4651 is hsa-miR-4651, miR-4665-3p is hsa-miR-4665-3p, miR-4674 is hsa-miR-4674, miR-4675 is hsa-miR-4675, miR-4689 is hsa-miR-4689, miR-4695-5p is hsa-miR-4695-5p, miR-4697-5p is hsa-miR-4697-5p, miR-4725-3p is hsa-miR-4725-3p, miR-4739 is hsa-miR-4739, miR-4745-5p is hsa-miR-4745-5p, miR-4763-3p is hsa-miR-4763-3p, miR-4792 is hsa-miR-4792, miR-486-3p is hsa-miR-486-3p, miR-5001-5p is hsa-miR-5001-5p, miR-5195-3p is hsa-miR-5195-3p, miR-550a-5p is hsa-miR-550a-5p, miR-5698 is hsa-miR-5698, miR-6075 is hsa-miR-6075, miR-6088 is hsa-miR-6088, miR-6089 is hsa-miR-6089, miR-6125 is hsa-miR-6125, miR-6126 is hsa-miR-6126, miR-614 is hsa-miR-614, miR-615-5p is hsa-miR-615-5p, miR-619-5p is hsa-miR-619-5p, miR-638 is hsa-miR-638, miR-642b-3p is hsa-miR-642b-3p, miR-650 is hsa-miR-650, miR-663a is hsa-miR-663a, miR-663b is hsa-miR-663b, miR-6717-5p is hsa-miR-6717-5p, miR-6721-5p is hsa-miR-6721-5p, miR-6726-5p is hsa-miR-6726-5p, miR-6727-5p is hsa-miR-6727-5p, miR-6738-5p is hsa-miR-6738-5p, miR-6741-5p is hsa-miR-6741-5p, miR-6749-5p is hsa-miR-6749-5p, miR-6752-5p is hsa-miR-6752-5p, miR-675-5p is hsa-miR-675-5p, miR-6757-5p is hsa-miR-6757-5p, miR-6763-5p is hsa-miR-6763-5p, miR-6765-5p is hsa-miR-6765-5p, miR-6775-5p is hsa-miR-6775-5p, miR-6780b-5p is hsa-miR-6780b-5p, miR-6782-5p is hsa-miR-6782-5p, miR-6784-5p is hsa-miR-6784-5p, miR-6800-5p is hsa-miR-6800-5p, miR-6806-5p is hsa-miR-6806-5p, miR-6840-3p is hsa-miR-6840-3p, miR-6848-5p is hsa-miR-6848-5p, miR-6851-5p is hsa-miR-6851-5p, miR-6870-5p is hsa-miR-6870-5p, miR-6872-3p is hsa-miR-6872-3p, miR-6875-5p is hsa-miR-6875-5p, miR-6877-5p is hsa-miR-6877-5p, miR-6879-5p is hsa-miR-6879-5p, miR-6880-5p is hsa-miR-6880-5p, miR-6885-5p is hsa-miR-6885-5p, miR-6887-5p is hsa-miR-6887-5p, miR-7108-5p is hsa-miR-7108-5p, miR-711 is hsa-miR-711, miR-7113-3p is hsa-miR-7113-3p, miR-744-5p is hsa-miR-744-5p, miR-760 is hsa-miR-760, miR-7845-5p is hsa-miR-7845-5p, miR-7847-3p is hsa-miR-7847-3p, miR-7977 is hsa-miR-7977, miR-8059 is hsa-miR-8059, miR-8063 is hsa-miR-8063, miR-8072 is hsa-miR-8072, miR-874-3p is hsa-miR-874-3p, miR-92a-2-5p is hsa-miR-92a-2-5p, miR-92b-5p is hsa-miR-92b-5p, miR-940 is hsa-miR-940, miR-1228-3p is hsa-miR-1228-3p, miR-1275 is hsa-miR-1275, miR-1307-3p is hsa-miR-1307-3p, miR-1343-5p is hsa-miR-1343-5p, miR-23a-3p is hsa-miR-23a-3p, miR-29b-3p is hsa-miR-29b-3p, miR-3135b is hsa-miR-3135b, miR-3185 is hsa-miR-3185, miR-4532 is hsa-miR-4532, miR-4690-5p is hsa-miR-4690-5p, miR-4758-5p is hsa-miR-4758-5p, miR-4783-3p is hsa-miR-4783-3p, miR-6131 is hsa-miR-6131, miR-625-3p is hsa-miR-625-3p, miR-6511a-5p is hsa-miR-6511a-5p, miR-6765-3p is hsa-miR-6765-3p, miR-6816-5p is hsa-miR-6816-5p, miR-6825-5p is hsa-miR-6825-5p, miR-6845-5p is hsa-miR-6845-5p, miR-7150 is hsa-miR-7150, miR-7641 is hsa-miR-7641, miR-7975 is hsa-miR-7975, and miR-92a-3p is hsa-miR-92a-3p.

### Definition of Terms

The terms used herein are defined as described below.

Abbreviations or terms such as nucleotide, polynucleotide, DNA, and RNA abide by “Guidelines for the preparation of specification which contain nucleotide and/or amino acid sequences” (edited by Japan Patent Office) and common use in the art.

The term “polynucleotide” used herein refers to a nucleic acid including any of RNA, DNA, and RNA/DNA (chimera). The DNA includes any of cDNA, genomic DNA, and synthetic DNA. The RNA includes any of total RNA, mRNA, rRNA, miRNA, siRNA, snoRNA, snRNA, non-coding RNA and synthetic RNA. Here the “synthetic DNA” and the “synthetic RNA” refer to a DNA and an RNA artificially prepared using, for example, an automatic nucleic acid synthesizer, on the basis of predetermined nucleotide sequences (which may be any of natural and non-natural sequences). The “non-natural sequence” is intended to be used in a broad sense and includes, for example, a sequence comprising substitution, deletion, insertion, and/or addition of one or more nucleotides (i.e., a variant sequence) and a sequence comprising one or more modified nucleotides (i.e., a modified sequence), which are different from the natural sequence. Herein, the term “polynucleotide” is used interchangeably with the term “nucleic acid.”

The term “fragment” used herein is a polynucleotide having a nucleotide sequence that consists of a consecutive portion of a polynucleotide and desirably has a length of 15 or more nucleotides, preferably 17 or more nucleotides, more preferably 19 or more nucleotides.

The term “gene” used herein is intended to include not only RNA and double-stranded DNA but also each single-stranded DNA such as a plus(+) strand (or a sense strand) or a complementary strand (or an antisense strand) constituting the duplex. The gene is not particularly limited by its length.

Thus, the “gene” used herein includes any of double-stranded DNA including human genomic DNA, single-stranded DNA (plus strand), single-stranded DNA having a sequence complementary to the plus strand (complementary strand), cDNA, microRNA (miRNA), their fragments, and human genome, and their transcripts, unless otherwise specified. The “gene” includes not only a “gene” represented by a particular nucleotide sequence (or SEQ ID NO) but “nucleic acids” encoding RNAs having biological functions equivalent to RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 1000 or a nucleotide sequence derived from the nucleotide sequence in which the nucleic acid u is replaced with t. Regardless whether or not there is a difference in functional region, the “gene” can comprise, for example, expression control regions, coding region, exons, or introns. The “gene” may be contained in a cell or may exist alone after being released from a cell. Alternatively, the “gene” may be in a state enclosed in a vesicle called exosome.

The term “exosome” used herein is a vesicle that is encapsulated by lipid bilayer and secreted from a cell. The exosome is derived from a multivesicular endosome and may incorporate biomaterials such as “genes” (e.g., RNA or DNA) or proteins when released into an extracellular environment. The exosome is known to be contained in a body fluid such as blood, serum, plasma or lymph.

The term “transcript” used herein refers to an RNA synthesized from the DNA sequence of a gene as a template. RNA polymerase binds to a site called promoter located upstream of the gene and adds ribonucleotides complementary to the nucleotide sequence of the DNA to the 3′ end to synthesize an RNA. This RNA contains not only the gene itself but the whole sequence from a transcription initiation site to the end of a polyA sequence, including expression control regions, coding region, exons, or introns.

Unless otherwise specified, the term “microRNA (miRNA)” used herein is intended to mean a 15- to 25-nucleotide non-coding RNA that is transcribed as an RNA precursor having a hairpin-like structure, cleaved by a dsRNA-cleaving enzyme having RNase III cleavage activity, and integrated into a protein complex called RISC, and that is involved in the suppression of translation of mRNA. The term “miRNA” used herein includes not only a “miRNA” represented by a particular nucleotide sequence (or SEQ ID NO) but a “miRNA” comprising a precursor of the “miRNA” (pre-miRNA or pri-miRNA) and having biological functions equivalent to miRNAs encoded by these, for example, a “miRNA” encoding a congener (i.e., a homolog or an ortholog), a variant such as a genetic polymorph, and a derivative. Such a “miRNA” encoding a precursor, a congener, a variant, or a derivative can be specifically identified using miRBase Release 21 (http://www.mirbase.org/), and examples thereof can include a “miRNA” having a nucleotide sequence hybridizing under stringent conditions described later to a complementary sequence of any particular nucleotide sequence represented by any of SEQ ID NOs: 1 to 1000. The term “miRNA” used herein may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

The term “probe” used herein includes a polynucleotide that is used for specifically detecting an RNA resulting from the expression of a gene or a polynucleotide derived from the RNA, and/or a polynucleotide complementary thereto.

The term “primer” used herein includes consecutive polynucleotides that specifically recognize and amplify an RNA resulting from the expression of a gene or a polynucleotide derived from the RNA, and/or a polynucleotide complementary thereto.

The term “complementary polynucleotide” or “polynucleotide consisting of a complementary nucleotide sequence” (complementary strand or reverse strand) used herein means a polynucleotide in a complementary relationship based on A:T (U) and G:C base pairs with the full-length sequence of the nucleotide sequence of the target polynucleotide or a nucleotide sequence derived from the nucleotide sequence in which the nucleic acid u is replaced with t, or a partial sequence thereof (herein, these nucleotide sequences are referred to as a plus strand for the sake of convenience). Such a complementary polynucleotide is not limited to a sequence completely complementary to the nucleotide sequence of the target plus strand and may have a complementary relationship to an extent that permits hybridization under stringent conditions to the target plus strand.

The term “stringent conditions” used herein refers to conditions under which a nucleic acid probe hybridizes to its target sequence to a detectably larger extent (e.g., a measurement value equal to or larger than “(a mean of background measurement values)+(a standard deviation of the background measurement values)×2”) than that for other sequences. The stringent conditions are dependent on a sequence and differ depending on an environment where hybridization is performed. A target sequence 100% complementary to the nucleic acid probe can be identified by controlling the stringency of hybridization and/or washing conditions. Specific examples of the “stringent conditions” will be mentioned later.

The term “Tm value” used herein means a temperature at which the double-stranded moiety of a polynucleotide is denatured into single strands so that the double strands and the single strands exist at a ratio of 1:1.

The term “variant” used herein means, in the case of a nucleic acid, a natural variant attributed to polymorphism, mutation, or the like; a variant containing the deletion, substitution, addition, or insertion of 1, 2 or 3 or more (e.g., 1 to several) nucleotides in a nucleotide sequence represented by a SEQ ID NO or a nucleotide sequence derived from the nucleotide sequence in which the nucleic acid u is replaced with t, or a partial sequence thereof; a variant containing the deletion, substitution, addition, or insertion of 1 or 2 or more nucleotides in a nucleotide sequence of a premature miRNA of the sequence of any of SEQ ID NOs 1 to 329 or a nucleotide sequence derived from the nucleotide sequence in which the nucleic acid u is replaced with t, or a partial sequence thereof; a variant that exhibits percent (%) identity of approximately 90% or higher, approximately 95% or higher, approximately 97% or higher, approximately 98% or higher, approximately 99% or higher to each of these nucleotide sequences or the partial sequences thereof; or a nucleic acid hybridizing under the stringent conditions defined above to a polynucleotide or an oligonucleotide comprising each of these nucleotide sequences or the partial sequences thereof.

The term “several” used herein means an integer of approximately 10, 9, 8, 7, 6, 5, 4, 3, or 2.

The variant used herein can be prepared by use of a well-known technique such as site-directed mutagenesis or mutagenesis using PCR.

The term “percent (%) identity” used herein can be determined with or without an introduced gap, using a protein or gene search system based on BLAST (https://blast.ncbi.nlm.nih.gov/Blast.cgi) or FASTA (http://www.genome.jp/tools/fasta/) (Zheng Zhang et al., 2000, J. Comput. Biol., Vol. 7, p. 203-214; Altschul, S. F. et al., 1990, Journal of Molecular Biology, Vol. 215, p. 403-410; and Pearson, W. R. et al., 1988, Proc. Natl. Acad. Sci. U.S.A, Vol. 85, p. 2444-2448).

The term “derivative” used herein is meant to include modified nucleic acids, unlimitedly for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen, P. E. et al., 1991, Science, Vol. 254, p. 1497-500), and LNA (locked nucleic acid; Obika, S. et al., 1998, Tetrahedron Lett., Vol. 39, p. 5401-5404).

As used herein, the “nucleic acid” capable of specifically binding to a polynucleotide selected from the lung cancer marker miRNAs described above or to a polynucleotide consisting of a nucleotide sequence complementary to that of the polynucleotide is a synthesized or prepared nucleic acid and, for example, includes a “nucleic acid probe” or a “primer” capable of detecting the polynucleotide. These nucleic acids are utilized directly or indirectly for detecting the presence or absence of lung cancer in a subject, for diagnosing the presence or absence or the severity of lung cancer, the presence or absence or the degree of amelioration of lung cancer, or the therapeutic sensitivity of lung cancer, or for screening for a candidate substance useful in the prevention, amelioration, or treatment of lung cancer. The “nucleic acid” includes a nucleotide, an oligonucleotide, and a polynucleotide capable of specifically recognizing and binding to a transcript represented by any of SEQ ID NOs: 1 to 1000 or a synthetic cDNA nucleic acid thereof in vivo, particularly, in a sample such as a body fluid (e.g., blood or urine), in relation to the development of lung cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” used herein is interchangeable with the term “examination”, “measurement”, “detection”, or “decision support”. As used herein, the term “evaluation” is meant to include diagnosing- or evaluation-supporting on the basis of examination results or measurement results.

The term “subject” used herein means a mammal such as a primate including a human and a chimpanzee, a pet animal including a dog and a cat, a livestock animal including cattle, a horse, sheep, and a goat, a rodent including a mouse and a rat, and animals raised in a zoo. The subject is preferably a human. The term “subject” herein may be optionally referred to as “test subject”. The term “healthy subject” also means such a mammal, which is an animal or a subject without the cancer to be detected. The healthy subject is preferably a human.

The “lung cancer” used herein is malignant tumor that develops in the lungs, and is broadly divided into small cell lung carcinoma and non-small cell lung carcinoma. The non-small cell lung carcinoma is generic name for lung cancer other than the small cell lung carcinoma and, for example, includes lung adenocarcinoma, lung squamous cell carcinoma, and large cell lung carcinoma.

The term “lung adenocarcinoma” or “adenocarcinoma” used herein is lung cancer in which differentiation into a duct of the gland or mucus production is found.

The term “lung squamous cell carcinoma” or “squamous cell carcinoma” used herein is lung cancer that exhibits cornification or intercellular bridge.

The term “large cell lung carcinoma” or “large cell carcinoma” used herein is lung cancer that is undifferentiated malignant epithelial tumor, and that is not categorized to any of small cell carcinoma, adenocarcinoma, and squamous cell carcinoma.

The term “small cell lung carcinoma” or “small cell carcinoma” used herein is lung cancer consisting of cells having a small size. The tumor cells exhibit a round, oval, or spindle form or the like with poor cytoplasms and unclear boundaries between the cells.

The term “other lung cancers” used herein is lung cancers other than adenocarcinoma, squamous cell carcinoma, large cell carcinoma, and small cell carcinoma and, for example, includes carcinoid tumor, adenosquamous carcinoma, polymorphic cell cancer, and salivary gland-type cancer.

The term “P” or “P value” used herein refers to a probability at which a more extreme statistic than that actually calculated from data under null hypothesis is observed in a statistical test. Thus, smaller “P” or “P value” is regarded as being a more significant difference between subjects to be compared.

The term “sensitivity” used herein means a value of (the number of true positives)/(the number of true positives+the number of false negatives). High sensitivity allows lung cancer to be detected early, leading to the complete resection of cancer sites and reduction in the rate of recurrence.

The term “specificity” used herein means a value of (the number of true negatives)/(the number of true negatives+the number of false positives). High specificity prevents needless extra examination for healthy subjects misjudged as being lung cancer patients, leading to reduction in burden on patients and reduction in medical expense.

The term “accuracy” used herein means a value of (the number of true positives+the number of true negatives)/(the total number of cases). The accuracy indicates the ratio of samples that are identified correctly to all samples, and serves as a primary index for evaluating detection performance.

As used herein, the “sample” that is subjected to determination, detection, or diagnosis refers to a tissue and a biological material in which the expression of the gene of the present invention varies as lung cancer develops, as lung cancer progresses, or as therapeutic effects on lung cancer are exerted. Specifically, the sample refers to a lung tissue, lymph node and a surrounding organ thereof, an organ suspected of having metastasis, the skin, a body fluid such as blood, urine, saliva, sweat, or tissue exudates, serum or plasma prepared from blood, feces, hair, and the like. The “sample” further refers to a biological sample extracted therefrom, specifically, a gene such as RNA or miRNA.

The term “hsa-miR-6787-5p gene” or “hsa-miR-6787-5p” used herein includes the hsa-miR-6787-5p gene (miRBase Accession No. MIMAT0027474) described in SEQ ID NO: 1, a homolog or an ortholog of a different organism species, and the like. The hsa-miR-6787-5p gene can be obtained by a method described in Ladewig E et al., 2012, Genome Res, Vol. 22, p. 1634-1645. Also, “hsa-mir-6787” (miRBase Accession No. MI0022632, SEQ ID NO: 330) having a hairpin-like structure is known as a precursor of “hsa-miR-6787-5p”.

The term “hsa-miR-920 gene” or “hsa-miR-920” used herein includes the hsa-miR-920 gene (miRBase Accession No. MIMAT0004970) described in SEQ ID NO: 2, a homolog or an ortholog of a different organism species, and the like. The hsa-miR-920 gene can be obtained by a method described in Novotny G W et al., 2007, Int J Androl, Vol. 30, p. 316-326. Also, “hsa-mir-920” (miRBase Accession No. MI0005712, SEQ ID NO: 331) having a hairpin-like structure is known as a precursor of “hsa-miR-920”.

The term “hsa-miR-3622a-5p gene” or “hsa-miR-3622a-5p” used herein includes the hsa-miR-3622a-5p gene (miRBase Accession No. MIMAT0018003) described in SEQ ID NO: 3, a homolog or an ortholog of a different organism species, and the like. The hsa-miR-3622a-5p gene can be obtained by a method described in Witten D et al., 2010, BMC Biol, Vol. 8, p. 58. Also, “hsa-mir-3622a” (miRBase Accession No. MI0016013, SEQ ID NO: 332) having a hairpin-like structure is known as a precursor of “hsa-miR-3622a-5p”.

The term “hsa-miR-1185-1-3p gene” or “hsa-miR-1185-1-3p” used herein includes the hsa-miR-1185-1-3p gene (miRBase Accession No. MIMAT0022838) described in SEQ ID NO: 4, a homolog or an ortholog of a different organism species, and the like. The hsa-miR-1185-1-3p gene can be obtained by a method described in Berezikov E et al., 2006, Genome Res, Vol. 16, p. 1289-1298. Also, “hsa-mir-1185-1” (miRBase Accession No. MI0003844, SEQ ID NO: 333) having a hairpin-like structure is known as a precursor of “hsa-miR-1185-1-3p”.

The term “hsa-miR-4327 gene” or “hsa-miR-4327” used herein includes the hsa-miR-4327 gene (miRBase Accession No. MIMAT0016889) described in SEQ ID NO: 5, a homolog or an ortholog of a different organism species, and the like. The hsa-miR-4327 gene can be obtained by a method described in Goff L A et al., 2009, PLoS One, Vol. 4, e7192. Also, “hsa-mir-4327” (miRBase Accession No. MI0015867, SEQ ID NO: 334) having a hairpin-like structure is known as a precursor of “hsa-miR-4327”.

The term “hsa-miR-5739 gene” or “hsa-miR-5739” used herein includes the hsa-miR-5739 gene (miRBase Accession No. MIMAT0023116) described in SEQ ID NO: 6, a homolog or an ortholog of a different organism species, and the like. The hsa-miR-5739 gene can be obtained by a method described in Yoo J K et al., 2011, Biochem Biophys Res Commun., Vol. 415, p. 258-262. Also, “hsa-mir-5739” (miRBase Accession No. MI0019412, SEQ ID NO: 335) having a hairpin-like structure is known as a precursor of “hsa-miR-5739”.

The term “hsa-miR-937-5p gene” or “hsa-miR-937-5p” used herein includes the hsa-miR-937-5p gene (miRBase Accession No. MIMAT0022938) described in SEQ ID NO: 7, a homolog or an ortholog of a different organism species, and the like. The hsa-miR-937-5p gene can be obtained by a method described in Lui W O et al., 2007, Cancer Res, Vol. 67, p. 6031-6043. Also, “hsa-mir-937” (miRBase Accession No. MI0005759, SEQ ID NO: 336) having a hairpin-like structure is known as a precursor of “hsa-miR-937-5p”.

The term “hsa-miR-1181 gene” or “hsa-miR-1181” used herein includes the hsa-miR-1181 gene (miRBase Accession No. MIMAT0005826) described in SEQ ID NO: 8, a homolog or an ortholog of a different organism species, and the like. The hsa-miR-1181 gene can be obtained by a method described in Subramanian S et al., 2008, Oncogene, Vol. 27, p. 2015-2026. Also, “hsa-mir-1181” (miRBase Accession No. MI0006274, SEQ ID NO: 337) having a hairpin-like structure is known as a precursor of “hsa-miR-1181”.

The term “hsa-miR-1185-2-3p gene” or “hsa-miR-1185-2-3p” used herein includes the hsa-miR-1185-2-3p gene (miRBase Accession No. MIMAT0022713) described in SEQ ID NO: 9, a homolog or an ortholog of a different organism species, and the like. The hsa-miR-1185-2-3p gene can be obtained by a method described in Berezikov E et al., 2006, Genome Res, Vol. 16, p. 1289-1298. Also, “hsa-mir-1185-2” (miRBase Accession No. MI0003821, SEQ ID NO: 338) having a hairpin-like structure is known as a precursor of “hsa-miR-1185-2-3p”.

The term “hsa-miR-1193 gene” or “hsa-miR-1193” used herein includes the hsa-miR-1193 gene (miRBase Accession No. MIMAT0015049) described in SEQ ID NO: 10, a homolog or an ortholog of a different organism species, and the like. The hsa-miR-1193 gene can be obtained by a method described in Stark M S et al., 2010, PLoS One, Vol. 5, e9685. Also, “hsa-mir-1193” (miRBase Accession No. MI0014205, SEQ ID NO: 339) having a hairpin-like structure is known as a precursor of “hsa-miR-1193”.

The term “hsa-miR-1207-5p gene” or “hsa-miR-1207-5p” used herein includes the hsa-miR-1207-5p gene (miRBase Accession No. MIMAT0005871) described in SEQ ID NO: 11, a homolog or an ortholog of a different organism species, and the like. The hsa-miR-1207-5p gene can be obtained by a method described in Huppi K et al., 2008, Mol Cancer Res, Vol. 6, p. 212-221. Also, “hsa-mir-1207” (miRBase Accession No. MI0006340, SEQ ID NO: 340) having a hairpin-like structure is known as a precursor of “hsa-miR-1207-5p”.

The term “hsa-miR-1238-5p gene” or “hsa-miR-1238-5p” used herein includes the hsa-miR-1238-5p gene (miRBase Accession No. MIMAT0022947) described in SEQ ID NO: 12, a homolog or an ortholog of a different organism species, and the like. The hsa-miR-1238-5p gene can be obtained by a method described in Berezikov E et al., 2007, Mol Cell, Vol. 28, p. 328-336. Also, “hsa-mir-1238” (miRBase Accession No. MI0006328, SEQ ID NO: 341) having a hairpin-like structure is known as a precursor of “hsa-miR-1238-5p”.

The term “hsa-miR-1246 gene” or “hsa-miR-1246” used herein includes the hsa-miR-1246 gene (miRBase Accession No. MIMAT0005898) described in SEQ ID NO: 13, a homolog or an ortholog of a different organism species, and the like. The hsa-miR-1246 gene can be obtained by a method described in Morin R D et al., 2008, Genome Res, Vol. 18, p. 610-621. Also, “hsa-mir-1246” (miRBase Accession No. MI0006381, SEQ ID NO: 342) having a hairpin-like structure is known as a precursor of “hsa-miR-1246”.

The term “hsa-miR-1249-5p gene” or “hsa-miR-1249-5p” used herein includes the hsa-miR-1249-5p gene (miRBase Accession No. MIMAT0032029) described in SEQ ID NO: 14, a homolog or an ortholog of a different organism species, and the like. The hsa-miR-1249-5p gene can be obtained by a method described in Morin R D et al., 2008, Genome Res, Vol. 18, p. 610-621. Also, “hsa-mir-1249” (miRBase Accession No. MI0006384, SEQ ID NO: 343) having a hairpin-like structure is known as a precursor of “hsa-miR-1249-5p”.

The term “hsa-miR-1292-3p gene” or “hsa-miR-1292-3p” used herein includes the hsa-miR-1292-3p gene (miRBase Accession No. MIMAT0022948) described in SEQ ID NO: 15, a homolog or an ortholog of a different organism species, and the like. The hsa-miR-1292-3p gene can be obtained by a method described in Morin R D et al., 2008, Genome Res, Vol. 18, p. 610-621. Also, “hsa-mir-1292” (miRBase Accession No. MI0006433, SEQ ID NO: 344) having a hairpin-like structure is known as a precursor of “hsa-miR-1292-3p”.

The term “hsa-miR-1469 gene” or “hsa-miR-1469” used herein includes the hsa-miR-1469 gene (miRBase Accession No. MIMAT0007347) described in SEQ ID NO: 16, a homolog or an ortholog of a different organism species, and the like. The hsa-miR-1469 gene can be obtained by a method described in Kawaji H et al., 2008, BMC Genomics, Vol. 9, p. 157. Also, “hsa-mir-1469” (miRBase Accession No. MI0007074, SEQ ID NO: 345) having a hairpin-like structure is known as a precursor of “hsa-miR-1469”.

The term “hsa-miR-1470 gene” or “hsa-miR-1470” used herein includes the hsa-miR-1470 gene (miRBase Accession No. MIMAT0007348) described in SEQ ID NO: 17, a homolog or an ortholog of a different organism species, and the like. The hsa-miR-1470 gene can be obtained by a method described in Kawaji H et al., 2008, BMC Genomics, Vol. 9, p. 157. Also, “hsa-mir-1470” (miRBase Accession No. MI0007075, SEQ ID NO: 346) having a hairpin-like structure is known as a precursor of “hsa-miR-1470”.

The term “hsa-miR-197-5p gene” or “hsa-miR-197-5p” used herein includes the hsa-miR-197-5p gene (miRBase Accession No. MIMAT0022691) described in SEQ ID NO: 18, a homolog or an ortholog of a different organism species, and the like. The hsa-miR-197-5p gene can be obtained by a method described in ‘Lagos-Quintana M et al., 2003, RNA, Vol. 9, p. 175-179′. Also, “hsa-mir-197” (miRBase Accession No. MI0000239, SEQ ID NO: 347) having a hairpin-like structure is known as a precursor of “hsa-miR-197-5p”.

The term “hsa-miR-208a-5p gene” or “hsa-miR-208a-5p” used herein includes the hsa-miR-208a-5p gene (miRBase Accession No. MIMAT0026474) described in SEQ ID NO: 19, a homolog or an ortholog of a different organism species, and the like. The hsa-miR-208a-5p gene can be obtained by a method described in Lagos-Quintana M et al., 2003, RNA, Vol. 9, p. 175-179. Also, “hsa-mir-208a” (miRBase Accession No. MI0000251, SEQ ID NO: 348) having a hairpin-like structure is known as a precursor of “hsa-miR-208a-5p”.

The term “hsa-miR-2110 gene” or “hsa-miR-2110” used herein includes the hsa-miR-2110 gene (miRBase Accession No. MIMAT0010133) described in SEQ ID NO: 20, a homolog or an ortholog of a different organism species, and the like. The hsa-miR-2110 gene can be obtained by a method described in Zhu J Y et al., 2009, J Virol, Vol. 83, p. 3333-3341. Also, “hsa-mir-2110” (miRBase Accession No. MI0010629, SEQ ID NO: 349) having a hairpin-like structure is known as a precursor of “hsa-miR-2110”.

The term “hsa-miR-211-3p gene” or “hsa-miR-211-3p” used herein includes the hsa-miR-211-3p gene (miRBase Accession No. MIMAT0022694) described in SEQ ID NO: 21, a homolog or an ortholog of a different organism species, and the like. The hsa-miR-211-3p gene can be obtained by a method described in Lim L P et al., 2003, Science, Vol. 299, p. 1540. Also, “hsa-mir-211” (miRBase Accession No. MI0000287, SEQ ID NO: 350) having a hairpin-like structure is known as a precursor of “hsa-miR-211-3p”.

The term “hsa-miR-2467-3p gene” or “hsa-miR-2467-3p” used herein includes the hsa-miR-2467-3p gene (miRBase Accession No. MIMAT0019953) described in SEQ ID NO: 22, a homolog or an ortholog of a different organism species, and the like. The hsa-miR-2467-3p gene can be obtained by a method described in Persson H et al., 2011, Cancer Res, Vol. 71, p. 78-86. Also, “hsa-mir-2467” (miRBase Accession No. MI0017432, SEQ ID NO: 351) having a hairpin-like structure is known as a precursor of “hsa-miR-2467-3p”.

The term “hsa-miR-3122 gene” or “hsa-miR-3122” used herein includes the hsa-miR-3122 gene (miRBase Accession No. MIMAT0014984) described in SEQ ID NO: 23, a homolog or an ortholog of a different organism species, and the like. The hsa-miR-3122 gene can be obtained by a method described in Stark M S et al., 2010, PLoS One., Vol. 5, e9685. Also, “hsa-mir-3122” (miRBase Accession No. MI0014138, SEQ ID NO: 352) having a hairpin-like structure is known as a precursor of “hsa-miR-3122”.

The term “hsa-miR-3141 gene” or “hsa-miR-3141” used herein includes the hsa-miR-3141 gene (miRBase Accession No. MIMAT0015010) described in SEQ ID NO: 24, a homolog or an ortholog of a different organism species, and the like. The hsa-miR-3141 gene can be obtained by a method described in Stark M S et al., 2010, PLoS One, Vol. 5, e9685. Also, “hsa-mir-3141” (miRBase Accession No. MI0014165, SEQ ID NO: 353) having a hairpin-like structure is known as a precursor of “hsa-miR-3141”.

The term “hsa-miR-3156-5p gene” or “hsa-miR-3156-5p” used herein includes the hsa-miR-3156-5p gene (miRBase Accession No. MIMAT0015030) described in SEQ ID NO: 25, a homolog or an ortholog of a different organism species, and the like. The hsa-miR-3156-5p gene can be obtained by a method described in Stark M S et al., 2010, PLoS One., Vol. 5, e9685. Also, “hsa-mir-3156-1, hsa-mir-3156-2, and hsa-mir-3156-3” (miRBase Accession Nos. MI0014184, MI0014230, and MI0014242, SEQ ID NOs: 354, 355, and 356) having a hairpin-like structure are known as precursors of “hsa-miR-3156-5p”.

The term “hsa-miR-3158-5p gene” or “hsa-miR-3158-5p” used herein includes the hsa-miR-3158-5p gene (miRBase Accession No. MIMAT0019211) described in SEQ ID NO: 26, a homolog or an ortholog of a different organism species, and the like. The hsa-miR-3158-5p gene can be obtained by a method described in Creighton C J et al., 2010, PLoS One, Vol. 5, e9637. Also, “hsa-mir-3158-1 and hsa-mir-3158-2” (miRBase Accession Nos. MI0014186 and MI0014187, SEQ ID NOs: 357 and 358) having a hairpin-like structure are known as precursors of “hsa-miR-3158-5p”.

The term “hsa-miR-3160-5p gene” or “hsa-miR-3160-5p” used herein includes the hsa-miR-3160-5p gene (miRBase Accession No. MIMAT0019212) described in SEQ ID NO: 27, a homolog or an ortholog of a different organism species, and the like. The hsa-miR-3160-5p gene can be obtained by a method described in Stark M S et al., 2010, PLoS One, Vol. 5, e9685. Also, “hsa-mir-3160-1 and hsa-mir-3160-2” (miRBase Accession Nos. MI0014189 and MI0014190, SEQ ID NOs: 359 and 360) having a hairpin-like structure are known as precursors of “hsa-miR-3160-5p”.

The term “hsa-miR-3180-3p gene” or “hsa-miR-3180-3p” used herein includes the hsa-miR-3180-3p gene (miRBase Accession No. MIMAT0015058) described in SEQ ID NO: 28, a homolog or an ortholog of a different organism species, and the like. The hsa-miR-3180-3p gene can be obtained by a method described in Creighton C J et al., 2010, PLoS One, Vol. 5, e9637. Also, “hsa-mir-3180-1, hsa-mir-3180-2, and hsa-mir-3180-3” (miRBase Accession Nos. MI0014214, MI0014215, and MI0014217, SEQ ID NOs: 361, 362, and 363) having a hairpin-like structure are known as precursors of “hsa-miR-3180-3p”.

The term “hsa-miR-3191-3p gene” or “hsa-miR-3191-3p” used herein includes the hsa-miR-3191-3p gene (miRBase Accession No. MIMAT0015075) described in SEQ ID NO: 29, a homolog or an ortholog of a different organism species, and the like. The hsa-miR-3191-3p gene can be obtained by a method described in Stark M S et al., 2010, PLoS One, Vol. 5, e9685. Also, “hsa-mir-3191” (miRBase Accession No. MI0014236, SEQ ID NO: 364) having a hairpin-like structure is known as a precursor of “hsa-miR-3191-3p”.

The term “hsa-miR-3194-3p gene” or “hsa-miR-3194-3p” used herein includes the hsa-miR-3194-3p gene (miRBase Accession No. MIMAT0019218) described in SEQ ID NO: 30, a homolog or an ortholog of a different organism species, and the like. The hsa-miR-3194-3p gene can be obtained by a method described in Stark M S et al., 2010, PLoS One, Vol. 5, e9685. Also, “hsa-mir-3194” (miRBase Accession No. MI0014239, SEQ ID NO: 365) having a hairpin-like structure is known as a precursor of “hsa-miR-3194-3p”.

The term “hsa-miR-320b gene” or “hsa-miR-320b” used herein includes the hsa-miR-320b gene (miRBase Accession No. MIMAT0005792) described in SEQ ID NO: 31, a homolog or an ortholog of a different organism species, and the like. The hsa-miR-320b gene can be obtained by a method described in Berezikov E et al., 2006, Genome Res, Vol. 16, p. 1289-1298. Also, “hsa-mir-320b-1 and hsa-mir-320b-2” (miRBase Accession Nos. MI0003776 and MI0003839, SEQ ID NOs: 366 and 367) having a hairpin-like structure are known as precursors of “hsa-miR-320b”.

The term “hsa-miR-328-5p gene” or “hsa-miR-328-5p” used herein includes the hsa-miR-328-5p gene (miRBase Accession No. MIMAT0026486) described in SEQ ID NO: 32, a homolog or an ortholog of a different organism species, and the like. The hsa-miR-328-5p gene can be obtained by a method described in Kim J et al., 2004, Proc Natl Acad Sci USA, Vol. 101, p. 360-365. Also, “hsa-mir-328” (miRBase Accession No. MI0000804, SEQ ID NO: 368) having a hairpin-like structure is known as a precursor of “hsa-miR-328-5p”.

The term “hsa-miR-3610 gene” or “hsa-miR-3610” used herein includes the hsa-miR-3610 gene (miRBase Accession No. MIMAT0017987) described in SEQ ID NO: 33, a homolog or an ortholog of a different organism species, and the like. The hsa-miR-3610 gene can be obtained by a method described in Witten D et al., 2010, BMC Biol., Vol. 8, 58. Also, “hsa-mir-3610” (miRBase Accession No. MI0016000, SEQ ID NO: 369) having a hairpin-like structure is known as a precursor of “hsa-miR-3610”.

The term “hsa-miR-3619-3p gene” or “hsa-miR-3619-3p” used herein includes the hsa-miR-3619-3p gene (miRBase Accession No. MIMAT0019219) described in SEQ ID NO: 34, a homolog or an ortholog of a different organism species, and the like. The hsa-miR-3619-3p gene can be obtained by a method described in Witten D et al., 2010, BMC Biol, Vol. 8, p. 58. Also, “hsa-mir-3619” (miRBase Accession No. MI0016009, SEQ ID NO: 370) having a hairpin-like structure is known as a precursor of “hsa-miR-3619-3p”.

The term “hsa-miR-3620-5p gene” or “hsa-miR-3620-5p” used herein includes the hsa-miR-3620-5p gene (miRBase Accession No. MIMAT0022967) described in SEQ ID NO: 35, a homolog or an ortholog of a different organism species, and the like. The hsa-miR-3620-5p gene can be obtained by a method described in Witten D et al., 2010, BMC Biol, Vol. 8, p. 58. Also, “hsa-mir-3620” (miRBase Accession No. MI0016011, SEQ ID NO: 371) having a hairpin-like structure is known as a precursor of “hsa-miR-3620-5p”.

The term “hsa-miR-370-3p gene” or “hsa-miR-370-3p” used herein includes the hsa-miR-370-3p gene (miRBase Accession No. MIMAT0000722) described in SEQ ID NO: 36, a homolog or an ortholog of a different organism species, and the like. The hsa-miR-370-3p gene can be obtained by a method described in Suh M R et al., 2004, Dev Biol., Vol. 270, 488-498. Also, “hsa-mir-370” (miRBase Accession No. MI0000778, SEQ ID NO: 372) having a hairpin-like structure is known as a precursor of “hsa-miR-370-3p”.

The term “hsa-miR-373-5p gene” or “hsa-miR-373-5p” used herein includes the hsa-miR-373-5p gene (miRBase Accession No. MIMAT0000725) described in SEQ ID NO: 37, a homolog or an ortholog of a different organism species, and the like. The hsa-miR-373-5p gene can be obtained by a method described in Suh M R et al., 2004, Dev Biol, Vol. 270, p. 488-498. Also, “hsa-mir-373” (miRBase Accession No. MI0000781, SEQ ID NO: 373) having a hairpin-like structure is known as a precursor of “hsa-miR-373-5p”.

The term “hsa-miR-3917 gene” or “hsa-miR-3917” used herein includes the hsa-miR-3917 gene (miRBase Accession No. MIMAT0018191) described in SEQ ID NO: 38, a homolog or an ortholog of a different organism species, and the like. The hsa-miR-3917 gene can be obtained by a method described in Creighton C J et al., 2010, PLoS One, Vol. 5, e9637. Also, “hsa-mir-3917” (miRBase Accession No. MI0016423, SEQ ID NO: 374) having a hairpin-like structure is known as a precursor of “hsa-miR-3917”.

The term “hsa-miR-3937 gene” or “hsa-miR-3937” used herein includes the hsa-miR-3937 gene (miRBase Accession No. MIMAT0018352) described in SEQ ID NO: 39, a homolog or an ortholog of a different organism species, and the like. The hsa-miR-3937 gene can be obtained by a method described in Liao J Y et al., 2010, PLoS One, Vol. 5, e10563. Also, “hsa-mir-3937” (miRBase Accession No. MI0016593, SEQ ID NO: 375) having a hairpin-like structure is known as a precursor of “hsa-miR-3937”.

The term “hsa-miR-4259 gene” or “hsa-miR-4259” used herein includes the hsa-miR-4259 gene (miRBase Accession No. MIMAT0016880) described in SEQ ID NO: 40, a homolog or an ortholog of a different organism species, and the like. The hsa-miR-4259 gene can be obtained by a method described in Goff L A et al., 2009, PLoS One, Vol. 4, e7192. Also, “hsa-mir-4259” (miRBase Accession No. MI0015858, SEQ ID NO: 376) having a hairpin-like structure is known as a precursor of “hsa-miR-4259”.

The term “hsa-miR-4281 gene” or “hsa-miR-4281” used herein includes the hsa-miR-4281 gene (miRBase Accession No. MIMAT0016907) described in SEQ ID NO: 41, a homolog or an ortholog of a different organism species, and the like. The hsa-miR-4281 gene can be obtained by a method described in Goff L A et al., 2009, PLoS One, Vol. 4, e7192. Also, “hsa-mir-4281” (miRBase Accession No. MI0015885, SEQ ID NO: 377) having a hairpin-like structure is known as a precursor of “hsa-miR-4281”.

The term “hsa-miR-4294 gene” or “hsa-miR-4294” used herein includes the hsa-miR-4294 gene (miRBase Accession No. MIMAT0016849) described in SEQ ID NO: 42, a homolog or an ortholog of a different organism species, and the like. The hsa-miR-4294 gene can be obtained by a method described in Goff L A et al., 2009, PLoS One, Vol. 4, e7192. Also, “hsa-mir-4294” (miRBase Accession No. MI0015827, SEQ ID NO: 378) having a hairpin-like structure is known as a precursor of “hsa-miR-4294”.

The term “hsa-miR-4419b gene” or “hsa-miR-4419b” used herein includes the hsa-miR-4419b gene (miRBase Accession No. MIMAT0019034) described in SEQ ID NO: 43, a homolog or an ortholog of a different organism species, and the like. The hsa-miR-4419b gene can be obtained by a method described in Jima D D et al., 2010, Blood, Vol. 116, e118-e127. Also, “hsa-mir-4419b” (miRBase Accession No. MI0016861, SEQ ID NO: 379) having a hairpin-like structure is known as a precursor of “hsa-miR-4419b”.

The term “hsa-miR-4428 gene” or “hsa-miR-4428” used herein includes the hsa-miR-4428 gene (miRBase Accession No. MIMAT0018943) described in SEQ ID NO: 44, a homolog or an ortholog of a different organism species, and the like. The hsa-miR-4428 gene can be obtained by a method described in Jima D D et al., 2010, Blood., Vol. 116, e118-e127. Also, “hsa-mir-4428” (miRBase Accession No. MI0016767, SEQ ID NO: 380) having a hairpin-like structure is known as a precursor of “hsa-miR-4428”.

The term “hsa-miR-4429 gene” or “hsa-miR-4429” used herein includes the hsa-miR-4429 gene (miRBase Accession No. MIMAT0018944) described in SEQ ID NO: 45, a homolog or an ortholog of a different organism species, and the like. The hsa-miR-4429 gene can be obtained by a method described in Jima D D et al., 2010, Blood, Vol. 116, e118-e127. Also, “hsa-mir-4429” (miRBase Accession No. MI0016768, SEQ ID NO: 381) having a hairpin-like structure is known as a precursor of “hsa-miR-4429”.

The term “hsa-miR-4433a-3p gene” or “hsa-miR-4433a-3p” used herein includes the hsa-miR-4433a-3p gene (miRBase Accession No. MIMAT0018949) described in SEQ ID NO: 46, a homolog or an ortholog of a different organism species, and the like. The hsa-miR-4433a-3p gene can be obtained by a method described in Jima D D et al., 2010, Blood, Vol. 116, e118-e127. Also, “hsa-mir-4433a” (miRBase Accession No. MI0016773, SEQ ID NO: 382) having a hairpin-like structure is known as a precursor of “hsa-miR-4433a-3p”.

The term “hsa-miR-4447 gene” or “hsa-miR-4447” used herein includes the hsa-miR-4447 gene (miRBase Accession No. MIMAT0018966) described in SEQ ID NO: 47, a homolog or an ortholog of a different organism species, and the like. The hsa-miR-4447 gene can be obtained by a method described in Jima D D et al., 2010, Blood, Vol. 116, e118-e127. Also, “hsa-mir-4447” (miRBase Accession No. MI0016790, SEQ ID NO: 383) having a hairpin-like structure is known as a precursor of “hsa-miR-4447”.

The term “hsa-miR-4449 gene” or “hsa-miR-4449” used herein includes the hsa-miR-4449 gene (miRBase Accession No. MIMAT0018968) described in SEQ ID NO: 48, a homolog or an ortholog of a different organism species, and the like. The hsa-miR-4449 gene can be obtained by a method described in Jima D D et al., 2010, Blood, Vol. 116, e118-e127. Also, “hsa-mir-4449” (miRBase Accession No. MI0016792, SEQ ID NO: 384) having a hairpin-like structure is known as a precursor of “hsa-miR-4449”.

The term “hsa-miR-4459 gene” or “hsa-miR-4459” used herein includes the hsa-miR-4459 gene (miRBase Accession No. MIMAT0018981) described in SEQ ID NO: 49, a homolog or an ortholog of a different organism species, and the like. The hsa-miR-4459 gene can be obtained by a method described in Jima D D et al., 2010, Blood, Vol. 116, e118-e127. Also, “hsa-mir-4459” (miRBase Accession No. MI0016805, SEQ ID NO: 385) having a hairpin-like structure is known as a precursor of “hsa-miR-4459”.

The term “hsa-miR-4480 gene” or “hsa-miR-4480” used herein includes the hsa-miR-4480 gene (miRBase Accession No. MIMAT0019014) described in SEQ ID NO: 50, a homolog or an ortholog of a different organism species, and the like. The hsa-miR-4480 gene can be obtained by a method described in Jima D D et al., 2010, Blood, Vol. 116, e118-e127. Also, “hsa-mir-4480” (miRBase Accession No. MI0016841, SEQ ID NO: 386) having a hairpin-like structure is known as a precursor of “hsa-miR-4480”.

The term “hsa-miR-4485-5p gene” or “hsa-miR-4485-5p” used herein includes the hsa-miR-4485-5p gene (miRBase Accession No. MIMAT0032116) described in SEQ ID NO: 51, a homolog or an ortholog of a different organism species, and the like. The hsa-miR-4485-5p gene can be obtained by a method described in Jima D D et al., 2010, Blood., Vol. 116, e118-e127. Also, “hsa-mir-4485” (miRBase Accession No. MI0016846, SEQ ID NO: 387) having a hairpin-like structure is known as a precursor of “hsa-miR-4485-5p”.

The term “hsa-miR-4486 gene” or “hsa-miR-4486” used herein includes the hsa-miR-4486 gene (miRBase Accession No. MIMAT0019020) described in SEQ ID NO: 52, a homolog or an ortholog of a different organism species, and the like. The hsa-miR-4486 gene can be obtained by a method described in Jima D D et al., 2010, Blood, Vol. 116, e118-e127. Also, “hsa-mir-4486” (miRBase Accession No. MI0016847, SEQ ID NO: 388) having a hairpin-like structure is known as a precursor of “hsa-miR-4486”.

The term “hsa-miR-4488 gene” or “hsa-miR-4488” used herein includes the hsa-miR-4488 gene (miRBase Accession No. MIMAT0019022) described in SEQ ID NO: 53, a homolog or an ortholog of a different organism species, and the like. The hsa-miR-4488 gene can be obtained by a method described in Jima D D et al., 2010, Blood, Vol. 116, e118-e127. Also, “hsa-mir-4488” (miRBase Accession No. MI0016849, SEQ ID NO: 389) having a hairpin-like structure is known as a precursor of “hsa-miR-4488”.

The term “hsa-miR-4489 gene” or “hsa-miR-4489” used herein includes the hsa-miR-4489 gene (miRBase Accession No. MIMAT0019023) described in SEQ ID NO: 54, a homolog or an ortholog of a different organism species, and the like. The hsa-miR-4489 gene can be obtained by a method described in Jima D D et al., 2010, Blood., Vol. 116, e118-e127. Also, “hsa-mir-4489” (miRBase Accession No. MI0016850, SEQ ID NO: 390) having a hairpin-like structure is known as a precursor of “hsa-miR-4489”.

The term “hsa-miR-4505 gene” or “hsa-miR-4505” used herein includes the hsa-miR-4505 gene (miRBase Accession No. MIMAT0019041) described in SEQ ID NO: 55, a homolog or an ortholog of a different organism species, and the like. The hsa-miR-4505 gene can be obtained by a method described in Jima D D et al., 2010, Blood, Vol. 116, e118-e127. Also, “hsa-mir-4505” (miRBase Accession No. MI0016868, SEQ ID NO: 391) having a hairpin-like structure is known as a precursor of “hsa-miR-4505”.

The term “hsa-miR-4513 gene” or “hsa-miR-4513” used herein includes the hsa-miR-4513 gene (miRBase Accession No. MIMAT0019050) described in SEQ ID NO: 56, a homolog or an ortholog of a different organism species, and the like. The hsa-miR-4513 gene can be obtained by a method described in Jima D D et al., 2010, Blood, Vol. 116, e118-e127. Also, “hsa-mir-4513” (miRBase Accession No. MI0016879, SEQ ID NO: 392) having a hairpin-like structure is known as a precursor of “hsa-miR-4513”.

The term “hsa-miR-4515 gene” or “hsa-miR-4515” used herein includes the hsa-miR-4515 gene (miRBase Accession No. MIMAT0019052) described in SEQ ID NO: 57, a homolog or an ortholog of a different organism species, and the like. The hsa-miR-4515 gene can be obtained by a method described in Jima D D et al., 2010, Blood, Vol. 116, e118-e127. Also, “hsa-mir-4515” (miRBase Accession No. MI0016881, SEQ ID NO: 393) having a hairpin-like structure is known as a precursor of “hsa-miR-4515”.

The term “hsa-miR-4530 gene” or “hsa-miR-4530” used herein includes the hsa-miR-4530 gene (miRBase Accession No. MIMAT0019069) described in SEQ ID NO: 58, a homolog or an ortholog of a different organism species, and the like. The hsa-miR-4530 gene can be obtained by a method described in Jima D D et al., 2010, Blood, Vol. 116, e118-e127. Also, “hsa-mir-4530” (miRBase Accession No. MI0016897, SEQ ID NO: 394) having a hairpin-like structure is known as a precursor of “hsa-miR-4530”.

The term “hsa-miR-4535 gene” or “hsa-miR-4535” used herein includes the hsa-miR-4535 gene (miRBase Accession No. MIMAT0019075) described in SEQ ID NO: 59, a homolog or an ortholog of a different organism species, and the like. The hsa-miR-4535 gene can be obtained by a method described in Jima D D et al., 2010, Blood., Vol. 116, e118-e127. Also, “hsa-mir-4535” (miRBase Accession No. MI0016903, SEQ ID NO: 395) having a hairpin-like structure is known as a precursor of “hsa-miR-4535”.

The term “hsa-miR-4635 gene” or “hsa-miR-4635” used herein includes the hsa-miR-4635 gene (miRBase Accession No. MIMAT0019692) described in SEQ ID NO: 60, a homolog or an ortholog of a different organism species, and the like. The hsa-miR-4635 gene can be obtained by a method described in Persson H et al., 2011, Cancer Res., Vol. 71, p. 78-86. Also, “hsa-mir-4635” (miRBase Accession No. MI0017262, SEQ ID NO: 396) having a hairpin-like structure is known as a precursor of “hsa-miR-4635”.

The term “hsa-miR-4640-5p gene” or “hsa-miR-4640-5p” used herein includes the hsa-miR-4640-5p gene (miRBase Accession No. MIMAT0019699) described in SEQ ID NO: 61, a homolog or an ortholog of a different organism species, and the like. The hsa-miR-4640-5p gene can be obtained by a method described in Persson H et al., 2011, Cancer Res, Vol. 71, p. 78-86. Also, “hsa-mir-4640” (miRBase Accession No. MI0017267, SEQ ID NO: 397) having a hairpin-like structure is known as a precursor of “hsa-miR-4640-5p”.

The term “hsa-miR-4646-5p gene” or “hsa-miR-4646-5p” used herein includes the hsa-miR-4646-5p gene (miRBase Accession No. MIMAT0019707) described in SEQ ID NO: 62, a homolog or an ortholog of a different organism species, and the like. The hsa-miR-4646-5p gene can be obtained by a method described in Persson H et al., 2011, Cancer Res., Vol. 71, p. 78-86. Also, “hsa-mir-4646” (miRBase Accession No. MI0017273, SEQ ID NO: 398) having a hairpin-like structure is known as a precursor of “hsa-miR-4646-5p”.

The term “hsa-miR-4656 gene” or “hsa-miR-4656” used herein includes the hsa-miR-4656 gene (miRBase Accession No. MIMAT0019723) described in SEQ ID NO: 63, a homolog or an ortholog of a different organism species, and the like. The hsa-miR-4656 gene can be obtained by a method described in Persson H et al., 2011, Cancer Res, Vol. 71, p. 78-86. Also, “hsa-mir-4656” (miRBase Accession No. MI0017284, SEQ ID NO: 399) having a hairpin-like structure is known as a precursor of “hsa-miR-4656”.

The term “hsa-miR-4663 gene” or “hsa-miR-4663” used herein includes the hsa-miR-4663 gene (miRBase Accession No. MIMAT0019735) described in SEQ ID NO: 64, a homolog or an ortholog of a different organism species, and the like. The hsa-miR-4663 gene can be obtained by a method described in Persson H. et al., 2011, Cancer Research, Vol. 71, p. 78-86. Also, “hsa-mir-4663” (miRBase Accession No. MI0017292, SEQ ID NO: 400) having a hairpin-like structure is known as a precursor of “hsa-miR-4663”.

The term “hsa-miR-4665-5p gene” or “hsa-miR-4665-5p” used herein includes the hsa-miR-4665-5p gene (miRBase Accession No. MIMAT0019739) described in SEQ ID NO: 65, a homolog or an ortholog of a different organism species, and the like. The hsa-miR-4665-5p gene can be obtained by a method described in Persson H et al., 2011, Cancer Res, Vol. 71, p. 78-86. Also, “hsa-mir-4665” (miRBase Accession No. MI0017295, SEQ ID NO: 401) having a hairpin-like structure is known as a precursor of “hsa-miR-4665-5p”.

The term “hsa-miR-4706 gene” or “hsa-miR-4706” used herein includes the hsa-miR-4706 gene (miRBase Accession No. MIMAT0019806) described in SEQ ID NO: 66, a homolog or an ortholog of a different organism species, and the like. The hsa-miR-4706 gene can be obtained by a method described in Persson H et al., 2011, Cancer Res, Vol. 71, p. 78-86. Also, “hsa-mir-4706” (miRBase Accession No. MI0017339, SEQ ID NO: 402) having a hairpin-like structure is known as a precursor of “hsa-miR-4706”.

The term “hsa-miR-4707-5p gene” or “hsa-miR-4707-5p” used herein includes the hsa-miR-4707-5p gene (miRBase Accession No. MIMAT0019807) described in SEQ ID NO: 67, a homolog or an ortholog of a different organism species, and the like. The hsa-miR-4707-5p gene can be obtained by a method described in Persson H et al., 2011, Cancer Res, Vol. 71, p. 78-86. Also, “hsa-mir-4707” (miRBase Accession No. MI0017340, SEQ ID NO: 403) having a hairpin-like structure is known as a precursor of “hsa-miR-4707-5p”.

The term “hsa-miR-4708-3p gene” or “hsa-miR-4708-3p” used herein includes the hsa-miR-4708-3p gene (miRBase Accession No. MIMAT0019810) described in SEQ ID NO: 68, a homolog or an ortholog of a different organism species, and the like. The hsa-miR-4708-3p gene can be obtained by a method described in Persson H et al., 2011, Cancer Res, Vol. 71, p. 78-86. Also, “hsa-mir-4708” (miRBase Accession No. MI0017341, SEQ ID NO: 404) having a hairpin-like structure is known as a precursor of “hsa-miR-4708-3p”.

The term “hsa-miR-4710 gene” or “hsa-miR-4710” used herein includes the hsa-miR-4710 gene (miRBase Accession No. MIMAT0019815) described in SEQ ID NO: 69, a homolog or an ortholog of a different organism species, and the like. The hsa-miR-4710 gene can be obtained by a method described in Persson H et al., 2011, Cancer Res, Vol. 71, p. 78-86. Also, “hsa-mir-4710” (miRBase Accession No. MI0017344, SEQ ID NO: 405) having a hairpin-like structure is known as a precursor of “hsa-miR-4710”.

The term “hsa-miR-4718 gene” or “hsa-miR-4718” used herein includes the hsa-miR-4718 gene (miRBase Accession No. MIMAT0019831) described in SEQ ID NO: 70, a homolog or an ortholog of a different organism species, and the like. The hsa-miR-4718 gene can be obtained by a method described in Persson H et al., 2011, Cancer Res, Vol. 71, p. 78-86. Also, “hsa-mir-4718” (miRBase Accession No. MI0017353, SEQ ID NO: 406) having a hairpin-like structure is known as a precursor of “hsa-miR-4718”.

The term “hsa-miR-4722-5p gene” or “hsa-miR-4722-5p” used herein includes the hsa-miR-4722-5p gene (miRBase Accession No. MIMAT0019836) described in SEQ ID NO: 71, a homolog or an ortholog of a different organism species, and the like. The hsa-miR-4722-5p gene can be obtained by a method described in Persson H et al., 2011, Cancer Res, Vol. 71, p. 78-86. Also, “hsa-mir-4722” (miRBase Accession No. MI0017357, SEQ ID NO: 407) having a hairpin-like structure is known as a precursor of “hsa-miR-4722-5p”.

The term “hsa-miR-4727-3p gene” or “hsa-miR-4727-3p” used herein includes the hsa-miR-4727-3p gene (miRBase Accession No. MIMAT0019848) described in SEQ ID NO: 72, a homolog or an ortholog of a different organism species, and the like. The hsa-miR-4727-3p gene can be obtained by a method described in Persson H et al., 2011, Cancer Res, Vol. 71, p. 78-86. Also, “hsa-mir-4727” (miRBase Accession No. MI0017364, SEQ ID NO: 408) having a hairpin-like structure is known as a precursor of “hsa-miR-4727-3p”.

The term “hsa-miR-4730 gene” or “hsa-miR-4730” used herein includes the hsa-miR-4730 gene (miRBase Accession No. MIMAT0019852) described in SEQ ID NO: 73, a homolog or an ortholog of a different organism species, and the like. The hsa-miR-4730 gene can be obtained by a method described in Persson H et al., 2011, Cancer Res, Vol. 71, p. 78-86. Also, “hsa-mir-4730” (miRBase Accession No. MI0017367, SEQ ID NO: 409) having a hairpin-like structure is known as a precursor of “hsa-miR-4730”.

The term “hsa-miR-4734 gene” or “hsa-miR-4734” used herein includes the hsa-miR-4734 gene (miRBase Accession No. MIMAT0019859) described in SEQ ID NO: 74, a homolog or an ortholog of a different organism species, and the like. The hsa-miR-4734 gene can be obtained by a method described in Persson H et al., 2011, Cancer Res, Vol. 71, p. 78-86. Also, “hsa-mir-4734” (miRBase Accession No. MI0017371, SEQ ID NO: 410) having a hairpin-like structure is known as a precursor of “hsa-miR-4734”.

The term “hsa-miR-4740-5p gene” or “hsa-miR-4740-5p” used herein includes the hsa-miR-4740-5p gene (miRBase Accession No. MIMAT0019869) described in SEQ ID NO: 75, a homolog or an ortholog of a different organism species, and the like. The hsa-miR-4740-5p gene can be obtained by a method described in Persson H et al., 2011, Cancer Res, Vol. 71, p. 78-86. Also, “hsa-mir-4740” (miRBase Accession No. MI0017378, SEQ ID NO: 411) having a hairpin-like structure is known as a precursor of “hsa-miR-4740-5p”.

The term “hsa-miR-4747-3p gene” or “hsa-miR-4747-3p” used herein includes the hsa-miR-4747-3p gene (miRBase Accession No. MIMAT0019883) described in SEQ ID NO: 76, a homolog or an ortholog of a different organism species, and the like. The hsa-miR-4747-3p gene can be obtained by a method described in Persson H et al., 2011, Cancer Res, Vol. 71, p. 78-86. Also, “hsa-mir-4747” (miRBase Accession No. MI0017386, SEQ ID NO: 412) having a hairpin-like structure is known as a precursor of “hsa-miR-4747-3p”.

The term “hsa-miR-4749-5p gene” or “hsa-miR-4749-5p” used herein includes the hsa-miR-4749-5p gene (miRBase Accession No. MIMAT0019885) described in SEQ ID NO: 77, a homolog or an ortholog of a different organism species, and the like. The hsa-miR-4749-5p gene can be obtained by a method described in Persson H et al., 2011, Cancer Res, Vol. 71, p. 78-86. Also, “hsa-mir-4749” (miRBase Accession No. MI0017388, SEQ ID NO: 413) having a hairpin-like structure is known as a precursor of “hsa-miR-4749-5p”.

The term “hsa-miR-4755-3p gene” or “hsa-miR-4755-3p” used herein includes the hsa-miR-4755-3p gene (miRBase Accession No. MIMAT0019896) described in SEQ ID NO: 78, a homolog or an ortholog of a different organism species, and the like. The hsa-miR-4755-3p gene can be obtained by a method described in Persson H. et al., 2011, Cancer Research, Vol. 71, p. 78-86. Also, “hsa-mir-4755” (miRBase Accession No. MI0017395, SEQ ID NO: 414) having a hairpin-like structure is known as a precursor of “hsa-miR-4755-3p”.

The term “hsa-miR-4763-5p gene” or “hsa-miR-4763-5p” used herein includes the hsa-miR-4763-5p gene (miRBase Accession No. MIMAT0019912) described in SEQ ID NO: 79, a homolog or an ortholog of a different organism species, and the like. The hsa-miR-4763-5p gene can be obtained by a method described in Persson H et al., 2011, Cancer Res., Vol. 71, p. 78-86. Also, “hsa-mir-4763” (miRBase Accession No. MI0017404, SEQ ID NO: 415) having a hairpin-like structure is known as a precursor of “hsa-miR-4763-5p”.

The term “hsa-miR-4787-3p gene” or “hsa-miR-4787-3p” used herein includes the hsa-miR-4787-3p gene (miRBase Accession No. MIMAT0019957) described in SEQ ID NO: 80, a homolog or an ortholog of a different organism species, and the like. The hsa-miR-4787-3p gene can be obtained by a method described in Persson H et al., 2011, Cancer Res, Vol. 71, p. 78-86. Also, “hsa-mir-4787” (miRBase Accession No. MI0017434, SEQ ID NO: 416) having a hairpin-like structure is known as a precursor of “hsa-miR-4787-3p”.

The term “hsa-miR-5008-5p gene” or “hsa-miR-5008-5p” used herein includes the hsa-miR-5008-5p gene (miRBase Accession No. MIMAT0021039) described in SEQ ID NO: 81, a homolog or an ortholog of a different organism species, and the like. The hsa-miR-5008-5p gene can be obtained by a method described in Hansen T B et al., 2011, RNA Biol, Vol. 8, p. 378-383. Also, “hsa-mir-5008” (miRBase Accession No. MI0017876, SEQ ID NO: 417) having a hairpin-like structure is known as a precursor of “hsa-miR-5008-5p”.

The term “hsa-miR-5010-5p gene” or “hsa-miR-5010-5p” used herein includes the hsa-miR-5010-5p gene (miRBase Accession No. MIMAT0021043) described in SEQ ID NO: 82, a homolog or an ortholog of a different organism species, and the like. The hsa-miR-5010-5p gene can be obtained by a method described in Hansen T B et al., 2011, RNA Biol, Vol. 8, p. 378-383. Also, “hsa-mir-5010” (miRBase Accession No. MI0017878, SEQ ID NO: 418) having a hairpin-like structure is known as a precursor of “hsa-miR-5010-5p”.

The term “hsa-miR-504-3p gene” or “hsa-miR-504-3p” used herein includes the hsa-miR-504-3p gene (miRBase Accession No. MIMAT0026612) described in SEQ ID NO: 83, a homolog or an ortholog of a different organism species, and the like. The hsa-miR-504-3p gene can be obtained by a method described in Bentwich I et al., 2005, Nat Genet, Vol. 37, p. 766-770. Also, “hsa-mir-504” (miRBase Accession No. MI0003189, SEQ ID NO: 419) having a hairpin-like structure is known as a precursor of “hsa-miR-504-3p”.

The term “hsa-miR-5090 gene” or “hsa-miR-5090” used herein includes the hsa-miR-5090 gene (miRBase Accession No. MIMAT0021082) described in SEQ ID NO: 84, a homolog or an ortholog of a different organism species, and the like. The hsa-miR-5090 gene can be obtained by a method described in Ding N et al., 2011, J Radiat Res, Vol. 52, p. 425-432. Also, “hsa-mir-5090” (miRBase Accession No. MI0017979, SEQ ID NO: 420) having a hairpin-like structure is known as a precursor of “hsa-miR-5090”.

The term “hsa-miR-5100 gene” or “hsa-miR-5100” used herein includes the hsa-miR-5100 gene (miRBase Accession No. MIMAT0022259) described in SEQ ID NO: 85, a homolog or an ortholog of a different organism species, and the like. The hsa-miR-5100 gene can be obtained by a method described in Tandon M et al., 2012, Oral Dis, Vol. 18, p. 127-131. Also, “hsa-mir-5100” (miRBase Accession No. MI0019116, SEQ ID NO: 421) having a hairpin-like structure is known as a precursor of “hsa-miR-5100”.

The term “hsa-miR-5196-5p gene” or “hsa-miR-5196-5p” used herein includes the hsa-miR-5196-5p gene (miRBase Accession No. MIMAT0021128) described in SEQ ID NO: 86, a homolog or an ortholog of a different organism species, and the like. The hsa-miR-5196-5p gene can be obtained by a method described in Schotte D et al., 2011, Leukemia, Vol. 25, p. 1389-1399. Also, “hsa-mir-5196” (miRBase Accession No. MI0018175, SEQ ID NO: 422) having a hairpin-like structure is known as a precursor of “hsa-miR-5196-5p”.

The term “hsa-miR-551b-5p gene” or “hsa-miR-551b-5p” used herein includes the hsa-miR-551b-5p gene (miRBase Accession No. MIMAT0004794) described in SEQ ID NO: 87, a homolog or an ortholog of a different organism species, and the like. The hsa-miR-551b-5p gene can be obtained by a method described in Cummins J M et al., 2006, Proc Natl Acad Sci U S A, Vol. 103, p. 3687-3692. Also, “hsa-mir-551b” (miRBase Accession No. MI0003575, SEQ ID NO: 423) having a hairpin-like structure is known as a precursor of “hsa-miR-551b-5p”.

The term “hsa-miR-557 gene” or “hsa-miR-557” used herein includes the hsa-miR-557 gene (miRBase Accession No. MIMAT0003221) described in SEQ ID NO: 88, a homolog or an ortholog of a different organism species, and the like. The hsa-miR-557 gene can be obtained by a method described in Cummins J M et al., 2006, Proc Natl Acad Sci USA, Vol. 103, p. 3687-3692. Also, “hsa-mir-557” (miRBase Accession No. M10003563, SEQ ID NO: 424) having a hairpin-like structure is known as a precursor of “hsa-miR-557”.

The term “hsa-miR-5787 gene” or “hsa-miR-5787” used herein includes the hsa-miR-5787 gene (miRBase Accession No. MIMAT0023252) described in SEQ ID NO: 89, a homolog or an ortholog of a different organism species, and the like. The hsa-miR-5787 gene can be obtained by a method described in Yoo H et al., 2011, Biochem Biophys Res Commun, Vol. 415, p. 567-572. Also, “hsa-mir-5787” (miRBase Accession No. MI0019797, SEQ ID NO: 425) having a hairpin-like structure is known as a precursor of “hsa-miR-5787”.

The term “hsa-miR-6090 gene” or “hsa-miR-6090” used herein includes the hsa-miR-6090 gene (miRBase Accession No. MIMAT0023715) described in SEQ ID NO: 90, a homolog or an ortholog of a different organism species, and the like. The hsa-miR-6090 gene can be obtained by a method described in Yoo J K et al., 2012, Stem Cells Dev, Vol. 21, p. 2049-2057. Also, “hsa-mir-6090” (miRBase Accession No. MI0020367, SEQ ID NO: 426) having a hairpin-like structure is known as a precursor of “hsa-miR-6090”.

The term “hsa-miR-6124 gene” or “hsa-miR-6124” used herein includes the hsa-miR-6124 gene (miRBase Accession No. MIMAT0024597) described in SEQ ID NO: 91, a homolog or an ortholog of a different organism species, and the like. The hsa-miR-6124 gene can be obtained by a method described in Smith J L et al., 2012, J Virol, Vol. 86, p. 5278-5287. Also, “hsa-mir-6124” (miRBase Accession No. MI0021258, SEQ ID NO: 427) having a hairpin-like structure is known as a precursor of “hsa-miR-6124”.

The term “hsa-miR-6132 gene” or “hsa-miR-6132” used herein includes the hsa-miR-6132 gene (miRBase Accession No. MIMAT0024616) described in SEQ ID NO: 92, a homolog or an ortholog of a different organism species, and the like. The hsa-miR-6132 gene can be obtained by a method described in Dannemann M et al., 2012, Genome Biol Evol, Vol. 4, p. 552-564. Also, “hsa-mir-6132” (miRBase Accession No. MI0021277, SEQ ID NO: 428) having a hairpin-like structure is known as a precursor of “hsa-miR-6132”.

The term “hsa-miR-6510-5p gene” or “hsa-miR-6510-5p” used herein includes the hsa-miR-6510-5p gene (miRBase Accession No. MIMAT0025476) described in SEQ ID NO: 93, a homolog or an ortholog of a different organism species, and the like. The hsa-miR-6510-5p gene can be obtained by a method described in Joyce C E et al., 2011, Hum Mol Genet, Vol. 20, p. 4025-4040. Also, “hsa-mir-6510” (miRBase Accession No. MI0022222, SEQ ID NO: 429) having a hairpin-like structure is known as a precursor of “hsa-miR-6510-5p”.

The term “hsa-miR-6511b-5p gene” or “hsa-miR-6511b-5p” used herein includes the hsa-miR-6511b-5p gene (miRBase Accession No. MIMAT0025847) described in SEQ ID NO: 94, a homolog or an ortholog of a different organism species, and the like. The hsa-miR-6511b-5p gene can be obtained by a method described in Li Y et al., 2012, Gene, Vol. 497, p. 330-335. Also, “hsa-mir-6511b-1 and hsa-mir-6511b-2” (miRBase Accession Nos. MI0022552 and MI0023431, SEQ ID NOs: 430 and 431) having a hairpin-like structure are known as precursors of “hsa-miR-6511b-5p”.

The term “hsa-miR-6515-3p gene” or “hsa-miR-6515-3p” used herein includes the hsa-miR-6515-3p gene (miRBase Accession No. MIMAT0025487) described in SEQ ID NO: 95, a homolog or an ortholog of a different organism species, and the like. The hsa-miR-6515-3p gene can be obtained by a method described in Joyce C E et al., 2011, Hum Mol Genet, Vol. 20, p. 4025-4040. Also, “hsa-mir-6515” (miRBase Accession No. MI0022227, SEQ ID NO: 432) having a hairpin-like structure is known as a precursor of “hsa-miR-6515-3p”.

The term “hsa-miR-654-5p gene” or “hsa-miR-654-5p” used herein includes the hsa-miR-654-5p gene (miRBase Accession No. MIMAT0003330) described in SEQ ID NO: 96, a homolog or an ortholog of a different organism species, and the like. The hsa-miR-654-5p gene can be obtained by a method described in Cummins J M et al., 2006, Proc Natl Acad Sci USA., Vol. 103, p. 3687-3692. Also, “hsa-mir-654” (miRBase Accession No. MI0003676, SEQ ID NO: 433) having a hairpin-like structure is known as a precursor of “hsa-miR-654-5p”.

The term “hsa-miR-658 gene” or “hsa-miR-658” used herein includes the hsa-miR-658 gene (miRBase Accession No. MIMAT0003336) described in SEQ ID NO: 97, a homolog or an ortholog of a different organism species, and the like. The hsa-miR-658 gene can be obtained by a method described in Cummins J M et al., 2006, Proc Natl Acad Sci USA, Vol. 103, p. 3687-3692. Also, “hsa-mir-658” (miRBase Accession No. MI0003682, SEQ ID NO: 434) having a hairpin-like structure is known as a precursor of “hsa-miR-658”.

The term “hsa-miR-668-5p gene” or “hsa-miR-668-5p” used herein includes the hsa-miR-668-5p gene (miRBase Accession No. MIMAT0026636) described in SEQ ID NO: 98, a homolog or an ortholog of a different organism species, and the like. The hsa-miR-668-5p gene can be obtained by a method described in Berezikov E et al., 2006, Genome Res., Vol. 16, p. 1289-1298. Also, “hsa-mir-668” (miRBase Accession No. MI0003761, SEQ ID NO: 435) having a hairpin-like structure is known as a precursor of “hsa-miR-668-5p”.

The term “hsa-miR-6722-5p gene” or “hsa-miR-6722-5p” used herein includes the hsa-miR-6722-5p gene (miRBase Accession No. MIMAT0025853) described in SEQ ID NO: 99, a homolog or an ortholog of a different organism species, and the like. The hsa-miR-6722-5p gene can be obtained by a method described in Li Y et al., 2012, Gene., Vol. 497, p. 330-335. Also, “hsa-mir-6722” (miRBase Accession No. MI0022557, SEQ ID NO: 436) having a hairpin-like structure is known as a precursor of “hsa-miR-6722-5p”.

The term “hsa-miR-6724-5p gene” or “hsa-miR-6724-5p” used herein includes the hsa-miR-6724-5p gene (miRBase Accession No. MIMAT0025856) described in SEQ ID NO: 100, a homolog or an ortholog of a different organism species, and the like. The hsa-miR-6724-5p gene can be obtained by a method described in Li Y et al., 2012, Gene, Vol. 497, p. 330-335. Also, “hsa-mir-6724” (miRBase Accession No. MI0022559, SEQ ID NO: 437) having a hairpin-like structure is known as a precursor of “hsa-miR-6724-5p”.

The term “hsa-miR-6729-3p gene” or “hsa-miR-6729-3p” used herein includes the hsa-miR-6729-3p gene (miRBase Accession No. MIMAT0027360) described in SEQ ID NO: 101, a homolog or an ortholog of a different organism species, and the like. The hsa-miR-6729-3p gene can be obtained by a method described in Ladewig E et al., 2012, Genome Res, Vol. 22, p. 1634-1645. Also, “hsa-mir-6729” (miRBase Accession No. MI0022574, SEQ ID NO: 438) having a hairpin-like structure is known as a precursor of “hsa-miR-6729-3p”.

The term “hsa-miR-6737-5p gene” or “hsa-miR-6737-5p” used herein includes the hsa-miR-6737-5p gene (miRBase Accession No. MIMAT0027375) described in SEQ ID NO: 102, a homolog or an ortholog of a different organism species, and the like. The hsa-miR-6737-5p gene can be obtained by a method described in Ladewig E et al., 2012, Genome Res, Vol. 22, p. 1634-1645. Also, “hsa-mir-6737” (miRBase Accession No. MI0022582, SEQ ID NO: 439) having a hairpin-like structure is known as a precursor of “hsa-miR-6737-5p”.

The term “hsa-miR-6756-5p gene” or “hsa-miR-6756-5p” used herein includes the hsa-miR-6756-5p gene (miRBase Accession No. MIMAT0027412) described in SEQ ID NO: 103, a homolog or an ortholog of a different organism species, and the like. The hsa-miR-6756-5p gene can be obtained by a method described in Ladewig E et al., 2012, Genome Res, Vol. 22, p. 1634-1645. Also, “hsa-mir-6756” (miRBase Accession No. MI0022601, SEQ ID NO: 440) having a hairpin-like structure is known as a precursor of “hsa-miR-6756-5p”.

The term “hsa-miR-6762-5p gene” or “hsa-miR-6762-5p” used herein includes the hsa-miR-6762-5p gene (miRBase Accession No. MIMAT0027424) described in SEQ ID NO: 104, a homolog or an ortholog of a different organism species, and the like. The hsa-miR-6762-5p gene can be obtained by a method described in Ladewig E et al., 2012, Genome Res, Vol. 22, p. 1634-1645. Also, “hsa-mir-6762” (miRBase Accession No. MI0022607, SEQ ID NO: 441) having a hairpin-like structure is known as a precursor of “hsa-miR-6762-5p”.

The term “hsa-miR-6763-3p gene” or “hsa-miR-6763-3p” used herein includes the hsa-miR-6763-3p gene (miRBase Accession No. MIMAT0027427) described in SEQ ID NO: 105, a homolog or an ortholog of a different organism species, and the like. The hsa-miR-6763-3p gene can be obtained by a method described in Ladewig E et al., 2012, Genome Res, Vol. 22, p. 1634-1645. Also, “hsa-mir-6763” (miRBase Accession No. MI0022608, SEQ ID NO: 442) having a hairpin-like structure is known as a precursor of “hsa-miR-6763-3p”.

The term “hsa-miR-6766-5p gene” or “hsa-miR-6766-5p” used herein includes the hsa-miR-6766-5p gene (miRBase Accession No. MIMAT0027432) described in SEQ ID NO: 106, a homolog or an ortholog of a different organism species, and the like. The hsa-miR-6766-5p gene can be obtained by a method described in Ladewig E et al., 2012, Genome Res, Vol. 22, p. 1634-1645. Also, “hsa-mir-6766” (miRBase Accession No. MI0022611, SEQ ID NO: 443) having a hairpin-like structure is known as a precursor of “hsa-miR-6766-5p”.

The term “hsa-miR-6769a-5p gene” or “hsa-miR-6769a-5p” used herein includes the hsa-miR-6769a-5p gene (miRBase Accession No. MIMAT0027438) described in SEQ ID NO: 107, a homolog or an ortholog of a different organism species, and the like. The hsa-miR-6769a-5p gene can be obtained by a method described in Ladewig E et al., 2012, Genome Res, Vol. 22, p. 1634-1645. Also, “hsa-mir-6769a” (miRBase Accession No. MI0022614, SEQ ID NO: 444) having a hairpin-like structure is known as a precursor of “hsa-miR-6769a-5p”.

The term “hsa-miR-6771-5p gene” or “hsa-miR-6771-5p” used herein includes the hsa-miR-6771-5p gene (miRBase Accession No. MIMAT0027442) described in SEQ ID NO: 108, a homolog or an ortholog of a different organism species, and the like. The hsa-miR-6771-5p gene can be obtained by a method described in Ladewig E et al., 2012, Genome Res, Vol. 22, p. 1634-1645. Also, “hsa-mir-6771” (miRBase Accession No. MI0022616, SEQ ID NO: 445) having a hairpin-like structure is known as a precursor of “hsa-miR-6771-5p”.

The term “hsa-miR-6786-5p gene” or “hsa-miR-6786-5p” used herein includes the hsa-miR-6786-5p gene (miRBase Accession No. MIMAT0027472) described in SEQ ID NO: 109, a homolog or an ortholog of a different organism species, and the like. The hsa-miR-6786-5p gene can be obtained by a method described in Ladewig E et al., 2012, Genome Res, Vol. 22, p. 1634-1645. Also, “hsa-mir-6786” (miRBase Accession No. MI0022631, SEQ ID NO: 446) having a hairpin-like structure is known as a precursor of “hsa-miR-6786-5p”.

The term “hsa-miR-6789-5p gene” or “hsa-miR-6789-5p” used herein includes the hsa-miR-6789-5p gene (miRBase Accession No. MIMAT0027478) described in SEQ ID NO: 110, a homolog or an ortholog of a different organism species, and the like. The hsa-miR-6789-5p gene can be obtained by a method described in Ladewig E et al., 2012, Genome Res., No. 22, p. 1634-1645. Also, “hsa-mir-6789” (miRBase Accession No. MI0022634, SEQ ID NO: 447) having a hairpin-like structure is known as a precursor of “hsa-miR-6789-5p”.

The term “hsa-miR-6794-5p gene” or “hsa-miR-6794-5p” used herein includes the hsa-miR-6794-5p gene (miRBase Accession No. MIMAT0027488) described in SEQ ID NO: 111, a homolog or an ortholog of a different organism species, and the like. The hsa-miR-6794-5p gene can be obtained by a method described in Ladewig E et al., 2012, Genome Res, Vol. 22, p. 1634-1645. Also, “hsa-mir-6794” (miRBase Accession No. MI0022639, SEQ ID NO: 448) having a hairpin-like structure is known as a precursor of “hsa-miR-6794-5p”.

The term “hsa-miR-6796-3p gene” or “hsa-miR-6796-3p” used herein includes the hsa-miR-6796-3p gene (miRBase Accession No. MIMAT0027493) described in SEQ ID NO: 112, a homolog or an ortholog of a different organism species, and the like. The hsa-miR-6796-3p gene can be obtained by a method described in Ladewig E et al., 2012, Genome Res, Vol. 22, p. 1634-1645. Also, “hsa-mir-6796” (miRBase Accession No. MI0022641, SEQ ID NO: 449) having a hairpin-like structure is known as a precursor of “hsa-miR-6796-3p”.

The term “hsa-miR-6797-5p gene” or “hsa-miR-6797-5p” used herein includes the hsa-miR-6797-5p gene (miRBase Accession No. MIMAT0027494) described in SEQ ID NO: 113, a homolog or an ortholog of a different organism species, and the like. The hsa-miR-6797-5p gene can be obtained by a method described in Ladewig E et al., 2012, Genome Res, Vol. 22, p. 1634-1645. Also, “hsa-mir-6797” (miRBase Accession No. MI0022642, SEQ ID NO: 450) having a hairpin-like structure is known as a precursor of “hsa-miR-6797-5p”.

The term “hsa-miR-6800-3p gene” or “hsa-miR-6800-3p” used herein includes the hsa-miR-6800-3p gene (miRBase Accession No. MIMAT0027501) described in SEQ ID NO: 114, a homolog or an ortholog of a different organism species, and the like. The hsa-miR-6800-3p gene can be obtained by a method described in Ladewig E et al., 2012, Genome Res, Vol. 22, p. 1634-1645. Also, “hsa-mir-6800” (miRBase Accession No. MI0022645, SEQ ID NO: 451) having a hairpin-like structure is known as a precursor of “hsa-miR-6800-3p”.

The term “hsa-miR-6802-5p gene” or “hsa-miR-6802-5p” used herein includes the hsa-miR-6802-5p gene (miRBase Accession No. MIMAT0027504) described in SEQ ID NO: 115, a homolog or an ortholog of a different organism species, and the like. The hsa-miR-6802-5p gene can be obtained by a method described in Ladewig E et al., 2012, Genome Res, Vol. 22, p. 1634-1645. Also, “hsa-mir-6802” (miRBase Accession No. MI0022647, SEQ ID NO: 452) having a hairpin-like structure is known as a precursor of “hsa-miR-6802-5p”.

The term “hsa-miR-6803-5p gene” or “hsa-miR-6803-5p” used herein includes the hsa-miR-6803-5p gene (miRBase Accession No. MIMAT0027506) described in SEQ ID NO: 116, a homolog or an ortholog of a different organism species, and the like. The hsa-miR-6803-5p gene can be obtained by a method described in Ladewig E et al., 2012, Genome Res, Vol. 22, p. 1634-1645. Also, “hsa-mir-6803” (miRBase Accession No. MI0022648, SEQ ID NO: 453) having a hairpin-like structure is known as a precursor of “hsa-miR-6803-5p”.

The term “hsa-miR-6805-3p gene” or “hsa-miR-6805-3p” used herein includes the hsa-miR-6805-3p gene (miRBase Accession No. MIMAT0027511) described in SEQ ID NO: 117, a homolog or an ortholog of a different organism species, and the like. The hsa-miR-6805-3p gene can be obtained by a method described in Ladewig E et al., 2012, Genome Res, Vol. 22, p. 1634-1645. Also, “hsa-mir-6805” (miRBase Accession No. MI0022650, SEQ ID NO: 454) having a hairpin-like structure is known as a precursor of “hsa-miR-6805-3p”.

The term “hsa-miR-6805-5p gene” or “hsa-miR-6805-5p” used herein includes the hsa-miR-6805-5p gene (miRBase Accession No. MIMAT0027510) described in SEQ ID NO: 118, a homolog or an ortholog of a different organism species, and the like. The hsa-miR-6805-5p gene can be obtained by a method described in Ladewig E et al., 2012, Genome Res, Vol. 22, p. 1634-1645. Also, “hsa-mir-6805” (miRBase Accession No. MI0022650, SEQ ID NO: 454) having a hairpin-like structure is known as a precursor of “hsa-miR-6805-5p”.

The term “hsa-miR-6807-5p gene” or “hsa-miR-6807-5p” used herein includes the hsa-miR-6807-5p gene (miRBase Accession No. MIMAT0027514) described in SEQ ID NO: 119, a homolog or an ortholog of a different organism species, and the like. The hsa-miR-6807-5p gene can be obtained by a method described in Ladewig E et al., 2012, Genome Res, Vol. 22, p. 1634-1645. Also, “hsa-mir-6807” (miRBase Accession No. MI0022652, SEQ ID NO: 455) having a hairpin-like structure is known as a precursor of “hsa-miR-6807-5p”.

The term “hsa-miR-6812-5p gene” or “hsa-miR-6812-5p” used herein includes the hsa-miR-6812-5p gene (miRBase Accession No. MIMAT0027524) described in SEQ ID NO: 120, a homolog or an ortholog of a different organism species, and the like. The hsa-miR-6812-5p gene can be obtained by a method described in Ladewig E et al., 2012, Genome Res, Vol. 22, p. 1634-1645. Also, “hsa-mir-6812” (miRBase Accession No. MI0022657, SEQ ID NO: 456) having a hairpin-like structure is known as a precursor of “hsa-miR-6812-5p”.

The term “hsa-miR-6819-5p gene” or “hsa-miR-6819-5p” used herein includes the hsa-miR-6819-5p gene (miRBase Accession No. MIMAT0027538) described in SEQ ID NO: 121, a homolog or an ortholog of a different organism species, and the like. The hsa-miR-6819-5p gene can be obtained by a method described in Ladewig E et al., 2012, Genome Res, Vol. 22, p. 1634-1645. Also, “hsa-mir-6819” (miRBase Accession No. MI0022664, SEQ ID NO: 457) having a hairpin-like structure is known as a precursor of “hsa-miR-6819-5p”.

The term “hsa-miR-6822-5p gene” or “hsa-miR-6822-5p” used herein includes the hsa-miR-6822-5p gene (miRBase Accession No. MIMAT0027544) described in SEQ ID NO: 122, a homolog or an ortholog of a different organism species, and the like. The hsa-miR-6822-5p gene can be obtained by a method described in Ladewig E et al., 2012, Genome Res, Vol. 22, p. 1634-1645. Also, “hsa-mir-6822” (miRBase Accession No. MI0022667, SEQ ID NO: 458) having a hairpin-like structure is known as a precursor of “hsa-miR-6822-5p”.

The term “hsa-miR-6824-5p gene” or “hsa-miR-6824-5p” used herein includes the hsa-miR-6824-5p gene (miRBase Accession No. MIMAT0027548) described in SEQ ID NO: 123, a homolog or an ortholog of a different organism species, and the like. The hsa-miR-6824-5p gene can be obtained by a method described in Ladewig E et al., 2012, Genome Res, Vol. 22, p. 1634-1645. Also, “hsa-mir-6824” (miRBase Accession No. MI0022669, SEQ ID NO: 459) having a hairpin-like structure is known as a precursor of “hsa-miR-6824-5p”.

The term “hsa-miR-6826-5p gene” or “hsa-miR-6826-5p” used herein includes the hsa-miR-6826-5p gene (miRBase Accession No. MIMAT0027552) described in SEQ ID NO: 124, a homolog or an ortholog of a different organism species, and the like. The hsa-miR-6826-5p gene can be obtained by a method described in Ladewig E et al., 2012, Genome Res, Vol. 22, p. 1634-1645. Also, “hsa-mir-6826” (miRBase Accession No. MI0022671, SEQ ID NO: 460) having a hairpin-like structure is known as a precursor of “hsa-miR-6826-5p”.

The term “hsa-miR-6850-5p gene” or “hsa-miR-6850-5p” used herein includes the hsa-miR-6850-5p gene (miRBase Accession No. MIMAT0027600) described in SEQ ID NO: 125, a homolog or an ortholog of a different organism species, and the like. The hsa-miR-6850-5p gene can be obtained by a method described in Ladewig E et al., 2012, Genome Res, Vol. 22, p. 1634-1645. Also, “hsa-mir-6850” (miRBase Accession No. MI0022696, SEQ ID NO: 461) having a hairpin-like structure is known as a precursor of “hsa-miR-6850-5p”.

The term “hsa-miR-6858-5p gene” or “hsa-miR-6858-5p” used herein includes the hsa-miR-6858-5p gene (miRBase Accession No. MIMAT0027616) described in SEQ ID NO: 126, a homolog or an ortholog of a different organism species, and the like. The hsa-miR-6858-5p gene can be obtained by a method described in Ladewig E et al., 2012, Genome Res, Vol. 22, p. 1634-1645. Also, “hsa-mir-6858” (miRBase Accession No. MI0022704, SEQ ID NO: 462) having a hairpin-like structure is known as a precursor of “hsa-miR-6858-5p”.

The term “hsa-miR-6861-5p gene” or “hsa-miR-6861-5p” used herein includes the hsa-miR-6861-5p gene (miRBase Accession No. MIMAT0027623) described in SEQ ID NO: 127, a homolog or an ortholog of a different organism species, and the like. The hsa-miR-6861-5p gene can be obtained by a method described in Ladewig E et al., 2012, Genome Res, Vol. 22, p. 1634-1645. Also, “hsa-mir-6861” (miRBase Accession No. MI0022708, SEQ ID NO: 463) having a hairpin-like structure is known as a precursor of “hsa-miR-6861-5p”.

The term “hsa-miR-6880-3p gene” or “hsa-miR-6880-3p” used herein includes the hsa-miR-6880-3p gene (miRBase Accession No. MIMAT0027661) described in SEQ ID NO: 128, a homolog or an ortholog of a different organism species, and the like. The hsa-miR-6880-3p gene can be obtained by a method described in Ladewig E et al., 2012, Genome Res, Vol. 22, p. 1634-1645. Also, “hsa-mir-6880” (miRBase Accession No. MI0022727, SEQ ID NO: 464) having a hairpin-like structure is known as a precursor of “hsa-miR-6880-3p”.

The term “hsa-miR-7107-5p gene” or “hsa-miR-7107-5p” used herein includes the hsa-miR-7107-5p gene (miRBase Accession No. MIMAT0028111) described in SEQ ID NO: 129, a homolog or an ortholog of a different organism species, and the like. The hsa-miR-7107-5p gene can be obtained by a method described in Ladewig E et al., 2012, Genome Res, Vol. 22, p. 1634-1645. Also, “hsa-mir-7107” (miRBase Accession No. MI0022958, SEQ ID NO: 465) having a hairpin-like structure is known as a precursor of “hsa-miR-7107-5p”.

The term “hsa-miR-7109-5p gene” or “hsa-miR-7109-5p” used herein includes the hsa-miR-7109-5p gene (miRBase Accession No. MIMAT0028115) described in SEQ ID NO: 130, a homolog or an ortholog of a different organism species, and the like. The hsa-miR-7109-5p gene can be obtained by a method described in Ladewig E et al., 2012, Genome Res, Vol. 22, p. 1634-1645. Also, “hsa-mir-7109” (miRBase Accession No. MI0022960, SEQ ID NO: 466) having a hairpin-like structure is known as a precursor of “hsa-miR-7109-5p”.

The term “hsa-miR-7114-5p gene” or “hsa-miR-7114-5p” used herein includes the hsa-miR-7114-5p gene (miRBase Accession No. MIMAT0028125) described in SEQ ID NO: 131, a homolog or an ortholog of a different organism species, and the like. The hsa-miR-7114-5p gene can be obtained by a method described in Ladewig E et al., 2012, Genome Res, Vol. 22, p. 1634-1645. Also, “hsa-mir-7114” (miRBase Accession No. MI0022965, SEQ ID NO: 467) having a hairpin-like structure is known as a precursor of “hsa-miR-7114-5p”.

The term “hsa-miR-7704 gene” or “hsa-miR-7704” used herein includes the hsa-miR-7704 gene (miRBase Accession No. MIMAT0030019) described in SEQ ID NO: 132, a homolog or an ortholog of a different organism species, and the like. The hsa-miR-7704 gene can be obtained by a method described in Swaminathan S et al., 2013, Biochem Biophys Res Commun, Vol. 434, p. 228-234. Also, “hsa-mir-7704” (miRBase Accession No. MI0025240, SEQ ID NO: 468) having a hairpin-like structure is known as a precursor of “hsa-miR-7704”.

The term “hsa-miR-7846-3p gene” or “hsa-miR-7846-3p” used herein includes the hsa-miR-7846-3p gene (miRBase Accession No. MIMAT0030421) described in SEQ ID NO: 133, a homolog or an ortholog of a different organism species, and the like. The hsa-miR-7846-3p gene can be obtained by a method described in Ple H et al., 2012, PLoS One., Vol. 7, e50746. Also, “hsa-mir-7846” (miRBase Accession No. MI0025516, SEQ ID NO: 469) having a hairpin-like structure is known as a precursor of “hsa-miR-7846-3p”.

The term “hsa-miR-8052 gene” or “hsa-miR-8052” used herein includes the hsa-miR-8052 gene (miRBase Accession No. MIMAT0030979) described in SEQ ID NO: 134, a homolog or an ortholog of a different organism species, and the like. The hsa-miR-8052 gene can be obtained by a method described in Wang H J et al., 2013, Shock., Vol. 39, 480-487. Also, “hsa-mir-8052” (miRBase Accession No. MI0025888, SEQ ID NO: 470) having a hairpin-like structure is known as a precursor of “hsa-miR-8052”.

The term “hsa-miR-8060 gene” or “hsa-miR-8060” used herein includes the hsa-miR-8060 gene (miRBase Accession No. MIMAT0030987) described in SEQ ID NO: 135, a homolog or an ortholog of a different organism species, and the like. The hsa-miR-8060 gene can be obtained by a method described in Wang H J et al., 2013, Shock., Vol. 39, 480-487. Also, “hsa-mir-8060” (miRBase Accession No. MI0025896, SEQ ID NO: 471) having a hairpin-like structure is known as a precursor of “hsa-miR-8060”.

The term “hsa-miR-8071 gene” or “hsa-miR-8071” used herein includes the hsa-miR-8071 gene (miRBase Accession No. MIMAT0030998) described in SEQ ID NO: 136, a homolog or an ortholog of a different organism species, and the like. The hsa-miR-8071 gene can be obtained by a method described in Wang H J et al., 2013, Shock, Vol. 39, p. 480-487. Also, “hsa-mir-8071-1 and hsa-mir-8071-2” (miRBase Accession Nos. MI0025907 and MI0026417, SEQ ID NOs: 472 and 473) having a hairpin-like structure are known as precursors of “hsa-miR-8071”.

The term “hsa-miR-8073 gene” or “hsa-miR-8073” used herein includes the hsa-miR-8073 gene (miRBase Accession No. MIMAT0031000) described in SEQ ID NO: 137, a homolog or an ortholog of a different organism species, and the like. The hsa-miR-8073 gene can be obtained by a method described in Wang H J et al., 2013, Shock, Vol. 39, p. 480-487. Also, “hsa-mir-8073” (miRBase Accession No. MI0025909, SEQ ID NO: 474) having a hairpin-like structure is known as a precursor of “hsa-miR-8073”.

The term “hsa-miR-874-5p gene” or “hsa-miR-874-5p” used herein includes the hsa-miR-874-5p gene (miRBase Accession No. MIMAT0026718) described in SEQ ID NO: 138, a homolog or an ortholog of a different organism species, and the like. The hsa-miR-874-5p gene can be obtained by a method described in Landgraf P et al., 2007, Cell., Vol. 129, p. 1401-1414. Also, “hsa-mir-874” (miRBase Accession No. MI0005532, SEQ ID NO: 475) having a hairpin-like structure is known as a precursor of “hsa-miR-874-5p”.

The term “hsa-miR-204-3p gene” or “hsa-miR-204-3p” used herein includes the hsa-miR-204-3p gene (miRBase Accession No. MIMAT0022693) described in SEQ ID NO: 139, a homolog or an ortholog of a different organism species, and the like. The hsa-miR-204-3p gene can be obtained by a method described in Lim L P et al., 2003, Science, Vol. 299, p. 1540. Also, “hsa-mir-204” (miRBase Accession No. MI0000284, SEQ ID NO: 476) having a hairpin-like structure is known as a precursor of “hsa-miR-204-3p”.

The term “hsa-miR-3154 gene” or “hsa-miR-3154” used herein includes the hsa-miR-3154 gene (miRBase Accession No. MIMAT0015028) described in SEQ ID NO: 140, a homolog or an ortholog of a different organism species, and the like. The hsa-miR-3154 gene can be obtained by a method described in Berezikov E et al., 2006, Genome Res, Vol. 16, p. 1289-1298. Also, “hsa-mir-3154” (miRBase Accession No. MI0014182, SEQ ID NO: 477) having a hairpin-like structure is known as a precursor of “hsa-miR-3154”.

The term “hsa-miR-3960 gene” or “hsa-miR-3960” used herein includes the hsa-miR-3960 gene (miRBase Accession No. MIMAT0019337) described in SEQ ID NO: 141, a homolog or an ortholog of a different organism species, and the like. The hsa-miR-3960 gene can be obtained by a method described in Hu R et al., 2011, J Biol Chem, Vol. 286, p. 12328-12339. Also, “hsa-mir-3960” (miRBase Accession No. MI0016964, SEQ ID NO: 478) having a hairpin-like structure is known as a precursor of “hsa-miR-3960”.

The term “hsa-miR-4433a-5p gene” or “hsa-miR-4433a-5p” used herein includes the hsa-miR-4433a-5p gene (miRBase Accession No. MIMAT0020956) described in SEQ ID NO: 142, a homolog or an ortholog of a different organism species, and the like. The hsa-miR-4433a-5p gene can be obtained by a method described in Jima D D et al., 2010, Blood, Vol. 116, e118-e127. Also, “hsa-mir-4433a” (miRBase Accession No. MI0016773, SEQ ID NO: 382) having a hairpin-like structure is known as a precursor of “hsa-miR-4433a-5p”.

The term “hsa-miR-4455 gene” or “hsa-miR-4455” used herein includes the hsa-miR-4455 gene (miRBase Accession No. MIMAT0018977) described in SEQ ID NO: 143, a homolog or an ortholog of a different organism species, and the like. The hsa-miR-4455 gene can be obtained by a method described in Jima D D et al., 2010, Blood., Vol. 116, e118-e127. Also, “hsa-mir-4455” (miRBase Accession No. MI0016801, SEQ ID NO: 479) having a hairpin-like structure is known as a precursor of “hsa-miR-4455”.

The term “hsa-miR-4462 gene” or “hsa-miR-4462” used herein includes the hsa-miR-4462 gene (miRBase Accession No. MIMAT0018986) described in SEQ ID NO: 144, a homolog or an ortholog of a different organism species, and the like. The hsa-miR-4462 gene can be obtained by a method described in Jima D D et al., 2010, Blood., Vol. 116, e118-e127. Also, “hsa-mir-4462” (miRBase Accession No. MI0016810, SEQ ID NO: 480) having a hairpin-like structure is known as a precursor of “hsa-miR-4462”.

The term “hsa-miR-4476 gene” or “hsa-miR-4476” used herein includes the hsa-miR-4476 gene (miRBase Accession No. MIMAT0019003) described in SEQ ID NO: 145, a homolog or an ortholog of a different organism species, and the like. The hsa-miR-4476 gene can be obtained by a method described in Jima D D et al., 2010, Blood, Vol. 116, e118-e127. Also, “hsa-mir-4476” (miRBase Accession No. MI0016828, SEQ ID NO: 481) having a hairpin-like structure is known as a precursor of “hsa-miR-4476”.

The term “hsa-miR-4508 gene” or “hsa-miR-4508” used herein includes the hsa-miR-4508 gene (miRBase Accession No. MIMAT0019045) described in SEQ ID NO: 146, a homolog or an ortholog of a different organism species, and the like. The hsa-miR-4508 gene can be obtained by a method described in Jima D D et al., 2010, Blood, Vol. 116, e118-e127. Also, “hsa-mir-4508” (miRBase Accession No. MI0016872, SEQ ID NO: 482) having a hairpin-like structure is known as a precursor of “hsa-miR-4508”.

The term “hsa-miR-4687-3p gene” or “hsa-miR-4687-3p” used herein includes the hsa-miR-4687-3p gene (miRBase Accession No. MIMAT0019775) described in SEQ ID NO: 147, a homolog or an ortholog of a different organism species, and the like. The hsa-miR-4687-3p gene can be obtained by a method described in Persson H et al., 2011, Cancer Res, Vol. 71, p. 78-86. Also, “hsa-mir-4687” (miRBase Accession No. MI0017319, SEQ ID NO: 483) having a hairpin-like structure is known as a precursor of “hsa-miR-4687-3p”.

The term “hsa-miR-4687-5p gene” or “hsa-miR-4687-5p” used herein includes the hsa-miR-4687-5p gene (miRBase Accession No. MIMAT0019774) described in SEQ ID NO: 148, a homolog or an ortholog of a different organism species, and the like. The hsa-miR-4687-5p gene can be obtained by a method described in Persson H et al., 2011, Cancer Res, Vol. 71, p. 78-86. Also, “hsa-mir-4687” (miRBase Accession No. MI0017319, SEQ ID NO: 483) having a hairpin-like structure is known as a precursor of “hsa-miR-4687-5p”.

The term “hsa-miR-4732-5p gene” or “hsa-miR-4732-5p” used herein includes the hsa-miR-4732-5p gene (miRBase Accession No. MIMAT0019855) described in SEQ ID NO: 149, a homolog or an ortholog of a different organism species, and the like. The hsa-miR-4732-5p gene can be obtained by a method described in Persson H et al., 2011, Cancer Res, Vol. 71, p. 78-86. Also, “hsa-mir-4732” (miRBase Accession No. MI0017369, SEQ ID NO: 484) having a hairpin-like structure is known as a precursor of “hsa-miR-4732-5p”.

The term “hsa-miR-4771 gene” or “hsa-miR-4771” used herein includes the hsa-miR-4771 gene (miRBase Accession No. MIMAT0019925) described in SEQ ID NO: 150, a homolog or an ortholog of a different organism species, and the like. The hsa-miR-4771 gene can be obtained by a method described in Persson H et al., 2011, Cancer Res, Vol. 71, p. 78-86. Also, “hsa-mir-4771-1 and hsa-mir-4771-2” (miRBase Accession Nos. MI0017412 and MI0017413, SEQ ID NOs: 485 and 486) having a hairpin-like structure are known as precursors of “hsa-miR-4771”.

The term “hsa-miR-642a-3p gene” or “hsa-miR-642a-3p” used herein includes the hsa-miR-642a-3p gene (miRBase Accession No. MIMAT0020924) described in SEQ ID NO: 151, a homolog or an ortholog of a different organism species, and the like. The hsa-miR-642a-3p gene can be obtained by a method described in Cummins J M et al., 2006, Proc Natl Acad Sci U S A, Vol. 103, p. 3687-3692. Also, “hsa-mir-642a” (miRBase Accession No. MI0003657, SEQ ID NO: 487) having a hairpin-like structure is known as a precursor of “hsa-miR-642a-3p”.

The term “hsa-miR-6732-5p gene” or “hsa-miR-6732-5p” used herein includes the hsa-miR-6732-5p gene (miRBase Accession No. MIMAT0027365) described in SEQ ID NO: 152, a homolog or an ortholog of a different organism species, and the like. The hsa-miR-6732-5p gene can be obtained by a method described in Ladewig E et al., 2012, Genome Res, Vol. 22, p. 1634-1645. Also, “hsa-mir-6732” (miRBase Accession No. MI0022577, SEQ ID NO: 488) having a hairpin-like structure is known as a precursor of “hsa-miR-6732-5p”.

The term “hsa-miR-6760-5p gene” or “hsa-miR-6760-5p” used herein includes the hsa-miR-6760-5p gene (miRBase Accession No. MIMAT0027420) described in SEQ ID NO: 153, a homolog or an ortholog of a different organism species, and the like. The hsa-miR-6760-5p gene can be obtained by a method described in Ladewig E et al., 2012, Genome Res., Vol. 22, p. 1634-1645. Also, “hsa-mir-6760” (miRBase Accession No. MI0022605, SEQ ID NO: 489) having a hairpin-like structure is known as a precursor of “hsa-miR-6760-5p”.

The term “hsa-miR-6799-5p gene” or “hsa-miR-6799-5p” used herein includes the hsa-miR-6799-5p gene (miRBase Accession No. MIMAT0027498) described in SEQ ID NO: 154, a homolog or an ortholog of a different organism species, and the like. The hsa-miR-6799-5p gene can be obtained by a method described in Ladewig E et al., 2012, Genome Res, Vol. 22, p. 1634-1645. Also, “hsa-mir-6799” (miRBase Accession No. MI0022644, SEQ ID NO: 490) having a hairpin-like structure is known as a precursor of “hsa-miR-6799-5p”.

The term “hsa-miR-6820-5p gene” or “hsa-miR-6820-5p” used herein includes the hsa-miR-6820-5p gene (miRBase Accession No. MIMAT0027540) described in SEQ ID NO: 155, a homolog or an ortholog of a different organism species, and the like. The hsa-miR-6820-5p gene can be obtained by a method described in Ladewig E et al., 2012, Genome Res, Vol. 22, p. 1634-1645. Also, “hsa-mir-6820” (miRBase Accession No. MI0022665, SEQ ID NO: 491) having a hairpin-like structure is known as a precursor of “hsa-miR-6820-5p”.

The term “hsa-miR-6821-5p gene” or “hsa-miR-6821-5p” used herein includes the hsa-miR-6821-5p gene (miRBase Accession No. MIMAT0027542) described in SEQ ID NO: 156, a homolog or an ortholog of a different organism species, and the like. The hsa-miR-6821-5p gene can be obtained by a method described in Ladewig E et al., 2012, Genome Res, Vol. 22, p. 1634-1645. Also, “hsa-mir-6821” (miRBase Accession No. MI0022666, SEQ ID NO: 492) having a hairpin-like structure is known as a precursor of “hsa-miR-6821-5p”.

The term “hsa-miR-6829-5p gene” or “hsa-miR-6829-5p” used herein includes the hsa-miR-6829-5p gene (miRBase Accession No. MIMAT0027558) described in SEQ ID NO: 157, a homolog or an ortholog of a different organism species, and the like. The hsa-miR-6829-5p gene can be obtained by a method described in Ladewig E et al., 2012, Genome Res, Vol. 22, p. 1634-1645. Also, “hsa-mir-6829” (miRBase Accession No. MI0022674, SEQ ID NO: 493) having a hairpin-like structure is known as a precursor of “hsa-miR-6829-5p”.

The term “hsa-miR-6893-5p gene” or “hsa-miR-6893-5p” used herein includes the hsa-miR-6893-5p gene (miRBase Accession No. MIMAT0027686) described in SEQ ID NO: 158, a homolog or an ortholog of a different organism species, and the like. The hsa-miR-6893-5p gene can be obtained by a method described in Ladewig E et al., 2012, Genome Res, Vol. 22, p. 1634-1645. Also, “hsa-mir-6893” (miRBase Accession No. MI0022740, SEQ ID NO: 494) having a hairpin-like structure is known as a precursor of “hsa-miR-6893-5p”.

The term “hsa-miR-7108-3p gene” or “hsa-miR-7108-3p” used herein includes the hsa-miR-7108-3p gene (miRBase Accession No. MIMAT0028114) described in SEQ ID NO: 159, a homolog or an ortholog of a different organism species, and the like. The hsa-miR-7108-3p gene can be obtained by a method described in Ladewig E et al., 2012, Genome Res, Vol. 22, p. 1634-1645. Also, “hsa-mir-7108” (miRBase Accession No. MI0022959, SEQ ID NO: 495) having a hairpin-like structure is known as a precursor of “hsa-miR-7108-3p”.

The term “hsa-miR-7111-5p gene” or “hsa-miR-7111-5p” used herein includes the hsa-miR-7111-5p gene (miRBase Accession No. MIMAT0028119) described in SEQ ID NO: 160, a homolog or an ortholog of a different organism species, and the like. The hsa-miR-7111-5p gene can be obtained by a method described in Ladewig E et al., 2012, Genome Res, Vol. 22, p. 1634-1645. Also, “hsa-mir-7111” (miRBase Accession No. MI0022962, SEQ ID NO: 496) having a hairpin-like structure is known as a precursor of “hsa-miR-7111-5p”.

The term “hsa-miR-8089 gene” or “hsa-miR-8089” used herein includes the hsa-miR-8089 gene (miRBase Accession No. MIMAT0031016) described in SEQ ID NO: 161, a homolog or an ortholog of a different organism species, and the like. The hsa-miR-8089 gene can be obtained by a method described in Wang H J et al., 2013, Shock, Vol. 39, p. 480-487. Also, “hsa-mir-8089” (miRBase Accession No. MI0025925, SEQ ID NO: 497) having a hairpin-like structure is known as a precursor of “hsa-miR-8089”.

The term “hsa-miR-885-3p gene” or “hsa-miR-885-3p” used herein includes the hsa-miR-885-3p gene (miRBase Accession No. MIMAT0004948) described in SEQ ID NO: 162, a homolog or an ortholog of a different organism species, and the like. The hsa-miR-885-3p gene can be obtained by a method described in Berezikov E et al., 2006, Genome Res, Vol. 16, p. 1289-1298. Also, “hsa-mir-885” (miRBase Accession No. MI0005560, SEQ ID NO: 498) having a hairpin-like structure is known as a precursor of “hsa-miR-885-3p”.

The term “hsa-miR-92b-3p gene” or “hsa-miR-92b-3p” used herein includes the hsa-miR-92b-3p gene (miRBase Accession No. MIMAT0003218) described in SEQ ID NO: 163, a homolog or an ortholog of a different organism species, and the like. The hsa-miR-92b-3p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 499) having a hairpin-like structure is known as a precursor of “hsa-miR-92b-3p”.

The term “hsa-miR-1343-3p gene” or “hsa-miR-1343-3p” used herein includes the hsa-miR-1343-3p gene (miRBase Accession No. MIMAT0019776) described in SEQ ID NO: 164, a homolog or an ortholog of a different organism species, and the like. The hsa-miR-1343-3p gene can be obtained by a method described in Persson H et al., 2011, Cancer Res, Vol. 71, p. 78-86. Also, “hsa-mir-1343” (miRBase Accession No. MI0017320, SEQ ID NO: 500) having a hairpin-like structure is known as a precursor of “hsa-miR-1343-3p”.

The term “hsa-miR-6746-5p gene” or “hsa-miR-6746-5p” used herein includes the hsa-miR-6746-5p gene (miRBase Accession No. MIMAT0027392) described in SEQ ID NO: 165, a homolog or an ortholog of a different organism species, and the like. The hsa-miR-6746-5p gene can be obtained by a method described in Ladewig E et al., 2012, Genome Res, Vol. 22, p. 1634-1645. Also, “hsa-mir-6746” (miRBase Accession No. MI0022591, SEQ ID NO: 501) having a hairpin-like structure is known as a precursor of “hsa-miR-6746-5p”.

The term “hsa-miR-422a gene” or “hsa-miR-422a” used herein includes the hsa-miR-422a gene (miRBase Accession No. MIMAT0001339) described in SEQ ID NO: 166, a homolog or an ortholog of a different organism species, and the like. The hsa-miR-422a gene can be obtained by a method described in Kasashima K et al., 2004, Biochem Biophys Res Commun, Vol. 322, p. 403-410. Also, “hsa-mir-422a” (miRBase Accession No. MI0001444, SEQ ID NO: 502) having a hairpin-like structure is known as a precursor of “hsa-miR-422a”.

The term “hsa-miR-187-5p gene” or “hsa-miR-187-5p” used herein includes the hsa-miR-187-5p gene (miRBase Accession No. MIMAT0004561) described in SEQ ID NO: 167, a homolog or an ortholog of a different organism species, and the like. The hsa-miR-187-5p gene can be obtained by a method described in Lim L P et al., 2003, Science, Vol. 299, p. 1540. Also, “hsa-mir-187” (miRBase Accession No. MI0000274, SEQ ID NO: 503) having a hairpin-like structure is known as a precursor of “hsa-miR-187-5p”.

The term “hsa-miR-4632-5p gene” or “hsa-miR-4632-5p” used herein includes the hsa-miR-4632-5p gene (miRBase Accession No. MIMAT0022977) described in SEQ ID NO: 168, a homolog or an ortholog of a different organism species, and the like. The hsa-miR-4632-5p gene can be obtained by a method described in Persson H et al., 2011, Cancer Res, Vol. 71, p. 78-86. Also, “hsa-mir-4632” (miRBase Accession No. MI0017259, SEQ ID NO: 504) having a hairpin-like structure is known as a precursor of “hsa-miR-4632-5p”.

The term “hsa-miR-6791-5p gene” or “hsa-miR-6791-5p” used herein includes the hsa-miR-6791-5p gene (miRBase Accession No. MIMAT0027482) described in SEQ ID NO: 169, a homolog or an ortholog of a different organism species, and the like. The hsa-miR-6791-5p gene can be obtained by a method described in Ladewig E et al., 2012, Genome Res, Vol. 22, p. 1634-1645. Also, “hsa-mir-6791” (miRBase Accession No. MI0022636, SEQ ID NO: 505) having a hairpin-like structure is known as a precursor of “hsa-miR-6791-5p”.

The term “hsa-miR-103a-3p gene” or “hsa-miR-103a-3p” used herein includes the hsa-miR-103a-3p gene (miRBase Accession No. MIMAT0000101) described in SEQ ID NO: 170, a homolog or an ortholog of a different organism species, and the like. The hsa-miR-103a-3p gene can be obtained by a method described in Mourelatos Z et al., 2002, Genes Dev. Vol. 16: p. 720-728. Also, “hsa-mir-103a-2 and hsa-mir-103a-1” (miRBase Accession Nos. MI0000109 and MI0000108, SEQ ID NOs: 506 and 507) having a hairpin-like structure are known as precursors of “hsa-miR-103a-3p”.

The term “hsa-miR-107 gene” or “hsa-miR-107” used herein includes the hsa-miR-107 gene (miRBase Accession No. MIMAT0000104) described in SEQ ID NO: 171, a homolog or an ortholog of a different organism species, and the like. The hsa-miR-107 gene can be obtained by a method described in Mourelatos Z et al., 2002, Genes Dev., Vol. 16, p. 720-728. Also, “hsa-mir-107” (miRBase Accession No. MI0000114, SEQ ID NO: 508) having a hairpin-like structure is known as a precursor of “hsa-miR-107”.

The term “hsa-miR-1199-5p gene” or “hsa-miR-1199-5p” used herein includes the hsa-miR-1199-5p gene (miRBase Accession No. MIMAT0031119) described in SEQ ID NO: 172, a homolog or an ortholog of a different organism species, and the like. The hsa-miR-1199-5p gene can be obtained by a method described in Salvi A et al., 2013, Int J Oncol, Vol. 42, p. 391-402. Also, “hsa-mir-1199” (miRBase Accession No. MI0020340, SEQ ID NO: 509) having a hairpin-like structure is known as a precursor of “hsa-miR-1199-5p”.

The term “hsa-miR-1225-3p gene” or “hsa-miR-1225-3p” used herein includes the hsa-miR-1225-3p gene (miRBase Accession No. MIMAT0005573) described in SEQ ID NO: 173, a homolog or an ortholog of a different organism species, and the like. The hsa-miR-1225-3p gene can be obtained by a method described in Berezikov E et al., 2007, Mol Cell, Vol. 28, p. 328-336. Also, “hsa-mir-1225” (miRBase Accession No. MI0006311, SEQ ID NO: 510) having a hairpin-like structure is known as a precursor of “hsa-miR-1225-3p”.

The term “hsa-miR-1225-5p gene” or “hsa-miR-1225-5p” used herein includes the hsa-miR-1225-5p gene (miRBase Accession No. MIMAT0005572) described in SEQ ID NO: 174, a homolog or an ortholog of a different organism species, and the like. The hsa-miR-1225-5p gene can be obtained by a method described in Berezikov E et al., 2007, Mol Cell, Vol. 28, p. 328-336. Also, “hsa-mir-1225” (miRBase Accession No. MI0006311, SEQ ID NO: 510) having a hairpin-like structure is known as a precursor of “hsa-miR-1225-5p”.

The term “hsa-miR-1228-5p gene” or “hsa-miR-1228-5p” used herein includes the hsa-miR-1228-5p gene (miRBase Accession No. MIMAT0005582) described in SEQ ID NO: 175, a homolog or an ortholog of a different organism species, and the like. The hsa-miR-1228-5p gene can be obtained by a method described in Berezikov E et al., 2007, Mol Cell, Vol. 28, p. 328-336. Also, “hsa-mir-1228” (miRBase Accession No. MI0006318, SEQ ID NO: 511) having a hairpin-like structure is known as a precursor of “hsa-miR-1228-5p”.

The term “hsa-miR-1229-5p gene” or “hsa-miR-1229-5p” used herein includes the hsa-miR-1229-5p gene (miRBase Accession No. MIMAT0022942) described in SEQ ID NO: 176, a homolog or an ortholog of a different organism species, and the like. The hsa-miR-1229-5p gene can be obtained by a method described in Berezikov E et al., 2007, Mol Cell, Vol. 28, p. 328-336. Also, “hsa-mir-1229” (miRBase Accession No. MI0006319, SEQ ID NO: 512) having a hairpin-like structure is known as a precursor of “hsa-miR-1229-5p”.

The term “hsa-miR-1233-5p gene” or “hsa-miR-1233-5p” used herein includes the hsa-miR-1233-5p gene (miRBase Accession No. MIMAT0022943) described in SEQ ID NO: 177, a homolog or an ortholog of a different organism species, and the like. The hsa-miR-1233-5p gene can be obtained by a method described in Berezikov E et al., 2007, Mol Cell, Vol. 28, p. 328-336. Also, “hsa-mir-1233-1 and hsa-mir-1233-2” (miRBase Accession Nos. MI0006323 and MI0015973, SEQ ID NOs: 513 and 514) having a hairpin-like structure are known as precursors of “hsa-miR-1233-5p”.

The term “hsa-miR-1237-5p gene” or “hsa-miR-1237-5p” used herein includes the hsa-miR-1237-5p gene (miRBase Accession No. MIMAT0022946) described in SEQ ID NO: 178, a homolog or an ortholog of a different organism species, and the like. The hsa-miR-1237-5p gene can be obtained by a method described in Berezikov E et al., 2007, Mol Cell, Vol. 28, p. 328-336. Also, “hsa-mir-1237” (miRBase Accession No. MI0006327, SEQ ID NO: 515) having a hairpin-like structure is known as a precursor of “hsa-miR-1237-5p”.

The term “hsa-miR-1247-3p gene” or “hsa-miR-1247-3p” used herein includes the hsa-miR-1247-3p gene (miRBase Accession No. MIMAT0022721) described in SEQ ID NO: 179, a homolog or an ortholog of a different organism species, and the like. The hsa-miR-1247-3p gene can be obtained by a method described in Morin R D et al., 2008, Genome Res, Vol. 18, p. 610-621. Also, “hsa-mir-1247” (miRBase Accession No. MI0006382, SEQ ID NO: 516) having a hairpin-like structure is known as a precursor of “hsa-miR-1247-3p”.

The term “hsa-miR-1249-3p gene” or “hsa-miR-1249-3p” used herein includes the hsa-miR-1249-3p gene (miRBase Accession No. MIMAT0005901) described in SEQ ID NO: 180, a homolog or an ortholog of a different organism species, and the like. The hsa-miR-1249-3p gene can be obtained by a method described in Morin R D et al., 2008, Genome Res, Vol. 18, p. 610-621. Also, “hsa-mir-1249” (miRBase Accession No. MI0006384, SEQ ID NO: 343) having a hairpin-like structure is known as a precursor of “hsa-miR-1249-3p”.

The term “hsa-miR-1254 gene” or “hsa-miR-1254” used herein includes the hsa-miR-1254 gene (miRBase Accession No. MIMAT0005905) described in SEQ ID NO: 181, a homolog or an ortholog of a different organism species, and the like. The hsa-miR-1254 gene can be obtained by a method described in Morin R D et al., 2008, Genome Res, Vol. 18, p. 610-621. Also, “hsa-mir-1254-1 and hsa-mir-1254-2” (miRBase Accession Nos. MI0006388 and MI0016747, SEQ ID NOs: 517 and 518) having a hairpin-like structure are known as precursors of “hsa-miR-1254”.

The term “hsa-miR-1260b gene” or “hsa-miR-1260b” used herein includes the hsa-miR-1260b gene (miRBase Accession No. MIMAT0015041) described in SEQ ID NO: 182, a homolog or an ortholog of a different organism species, and the like. The hsa-miR-1260b gene can be obtained by a method described in Stark M S et al., 2010, PLoS One, Vol. 5, e9685. Also, “hsa-mir-1260b” (miRBase Accession No. MI0014197, SEQ ID NO: 519) having a hairpin-like structure is known as a precursor of “hsa-miR-1260b”.

The term “hsa-miR-1268a gene” or “hsa-miR-1268a” used herein includes the hsa-miR-1268a gene (miRBase Accession No. MIMAT0005922) described in SEQ ID NO: 183, a homolog or an ortholog of a different organism species, and the like. The hsa-miR-1268a gene can be obtained by a method described in Morin R D et al., 2008, Genome Res, Vol. 18, p. 610-621. Also, “hsa-mir-1268a” (miRBase Accession No. MI0006405, SEQ ID NO: 520) having a hairpin-like structure is known as a precursor of “hsa-miR-1268a”.

The term “hsa-miR-1268b gene” or “hsa-miR-1268b” used herein includes the hsa-miR-1268b gene (miRBase Accession No. MIMAT0018925) described in SEQ ID NO: 184, a homolog or an ortholog of a different organism species, and the like. The hsa-miR-1268b gene can be obtained by a method described in Jima D D et al., 2010, Blood, Vol. 116, e118-e127. Also, “hsa-mir-1268b” (miRBase Accession No. MI0016748, SEQ ID NO: 521) having a hairpin-like structure is known as a precursor of “hsa-miR-1268b”.

The term “hsa-miR-1273g-3p gene” or “hsa-miR-1273g-3p” used herein includes the hsa-miR-1273g-3p gene (miRBase Accession No. MIMAT0022742) described in SEQ ID NO: 185, a homolog or an ortholog of a different organism species, and the like. The hsa-miR-1273g-3p gene can be obtained by a method described in Reshmi G et al., 2011, Genomics, Vol. 97, p. 333-340. Also, “hsa-mir-1273g” (miRBase Accession No. MI0018003, SEQ ID NO: 522) having a hairpin-like structure is known as a precursor of “hsa-miR-1273g-3p”.

The term “hsa-miR-128-1-5p gene” or “hsa-miR-128-1-5p” used herein includes the hsa-miR-128-1-5p gene (miRBase Accession No. MIMAT0026477) described in SEQ ID NO: 186, a homolog or an ortholog of a different organism species, and the like. The hsa-miR-128-1-5p gene can be obtained by a method described in Lagos-Quintana M et al., 2002, Curr Biol, Vol. 12, p. 735-739. Also, “hsa-mir-128-1” (miRBase Accession No. MI0000447, SEQ ID NO: 523) having a hairpin-like structure is known as a precursor of “hsa-miR-128-1-5p”.

The term “hsa-miR-128-2-5p gene” or “hsa-miR-128-2-5p” used herein includes the hsa-miR-128-2-5p gene (miRBase Accession No. MIMAT0031095) described in SEQ ID NO: 187, a homolog or an ortholog of a different organism species, and the like. The hsa-miR-128-2-5p gene can be obtained by a method described in Lagos-Quintana M et al., 2002, Curr Biol, Vol. 12, p. 735-739. Also, “hsa-mir-128-2” (miRBase Accession No. M10000727, SEQ ID NO: 524) having a hairpin-like structure is known as a precursor of “hsa-miR-128-2-5p”.

The term “hsa-miR-1290 gene” or “hsa-miR-1290” used herein includes the hsa-miR-1290 gene (miRBase Accession No. MIMAT0005880) described in SEQ ID NO: 188, a homolog or an ortholog of a different organism species, and the like. The hsa-miR-1290 gene can be obtained by a method described in Morin R D et al., 2008, Genome Res, Vol. 18, p. 610-621. Also, “hsa-mir-1290” (miRBase Accession No. MI0006352, SEQ ID NO: 525) having a hairpin-like structure is known as a precursor of “hsa-miR-1290”.

The term “hsa-miR-150-3p gene” or “hsa-miR-150-3p” used herein includes the hsa-miR-150-3p gene (miRBase Accession No. MIMAT0004610) described in SEQ ID NO: 189, a homolog or an ortholog of a different organism species, and the like. The hsa-miR-150-3p gene can be obtained by a method described in Lagos-Quintana M et al., 2002, Curr Biol, Vol. 12, p. 735-739. Also, “hsa-mir-150” (miRBase Accession No. MI0000479, SEQ ID NO: 526) having a hairpin-like structure is known as a precursor of “hsa-miR-150-3p”.

The term “hsa-miR-17-3p gene” or “hsa-miR-17-3p” used herein includes the hsa-miR-17-3p gene (miRBase Accession No. MIMAT0000071) described in SEQ ID NO: 190, a homolog or an ortholog of a different organism species, and the like. The hsa-miR-17-3p gene can be obtained by a method described in Lagos-Quintana M et al., 2001, Science., Vol. 294, p. 853-858. Also, “hsa-mir-17” (miRBase Accession No. MI0000071, SEQ ID NO: 527) having a hairpin-like structure is known as a precursor of “hsa-miR-17-3p”.

The term “hsa-miR-1908-5p gene” or “hsa-miR-1908-5p” used herein includes the hsa-miR-1908-5p gene (miRBase Accession No. MIMAT0007881) described in SEQ ID NO: 191, a homolog or an ortholog of a different organism species, and the like. The hsa-miR-1908-5p gene can be obtained by a method described in Bar M et al., 2008, Stem Cells, Vol. 26, p. 2496-2505. Also, “hsa-mir-1908” (miRBase Accession No. MI0008329, SEQ ID NO: 528) having a hairpin-like structure is known as a precursor of “hsa-miR-1908-5p”.

The term “hsa-miR-1909-3p gene” or “hsa-miR-1909-3p” used herein includes the hsa-miR-1909-3p gene (miRBase Accession No. MIMAT0007883) described in SEQ ID NO: 192, a homolog or an ortholog of a different organism species, and the like. The hsa-miR-1909-3p gene can be obtained by a method described in Bar M et al., 2008, Stem Cells, Vol. 26, p. 2496-2505. Also, “hsa-mir-1909” (miRBase Accession No. MI0008330, SEQ ID NO: 529) having a hairpin-like structure is known as a precursor of “hsa-miR-1909-3p”.

The term “hsa-miR-1914-3p gene” or “hsa-miR-1914-3p” used herein includes the hsa-miR-1914-3p gene (miRBase Accession No. MIMAT0007890) described in SEQ ID NO: 193, a homolog or an ortholog of a different organism species, and the like. The hsa-miR-1914-3p gene can be obtained by a method described in Bar M et al., 2008, Stem Cells, Vol. 26, p. 2496-2505. Also, “hsa-mir-1914” (miRBase Accession No. MI0008335, SEQ ID NO: 530) having a hairpin-like structure is known as a precursor of “hsa-miR-1914-3p”.

The term “hsa-miR-1915-3p gene” or “hsa-miR-1915-3p” used herein includes the hsa-miR-1915-3p gene (miRBase Accession No. MIMAT0007892) described in SEQ ID NO: 194, a homolog or an ortholog of a different organism species, and the like. The hsa-miR-1915-3p gene can be obtained by a method described in Bar M et al., 2008, Stem Cells, Vol. 26, p. 2496-2505. Also, “hsa-mir-1915” (miRBase Accession No. MI0008336, SEQ ID NO: 531) having a hairpin-like structure is known as a precursor of “hsa-miR-1915-3p”.

The term “hsa-miR-191-5p gene” or “hsa-miR-191-5p” used herein includes the hsa-miR-191-5p gene (miRBase Accession No. MIMAT0000440) described in SEQ ID NO: 195, a homolog or an ortholog of a different organism species, and the like. The hsa-miR-191-5p gene can be obtained by a method described in Lagos-Quintana M et al., 2003, RNA, Vol. 9, p. 175-179. Also, “hsa-mir-191” (miRBase Accession No. MI0000465, SEQ ID NO: 532) having a hairpin-like structure is known as a precursor of “hsa-miR-191-5p”.

The term “hsa-miR-22-3p gene” or “hsa-miR-22-3p” used herein includes the hsa-miR-22-3p gene (miRBase Accession No. MIMAT0000077) described in SEQ ID NO: 196, a homolog or an ortholog of a different organism species, and the like. The hsa-miR-22-3p gene can be obtained by a method described in Lagos-Quintana M et al., 2001, Science, Vol. 294, p. 853-858. Also, “hsa-mir-22” (miRBase Accession No. MI0000078, SEQ ID NO: 533) having a hairpin-like structure is known as a precursor of “hsa-miR-22-3p”.

The term “hsa-miR-23b-3p gene” or “hsa-miR-23b-3p” used herein includes the hsa-miR-23b-3p gene (miRBase Accession No. MIMAT0000418) described in SEQ ID NO: 197, a homolog or an ortholog of a different organism species, and the like. The hsa-miR-23b-3p gene can be obtained by a method described in Lagos-Quintana M et al., 2002, Curr Biol, Vol. 12, p. 735-739. Also, “hsa-mir-23b” (miRBase Accession No. MI0000439, SEQ ID NO: 534) having a hairpin-like structure is known as a precursor of “hsa-miR-23b-3p”.

The term “hsa-miR-24-3p gene” or “hsa-miR-24-3p” used herein includes the hsa-miR-24-3p gene (miRBase Accession No. MIMAT0000080) described in SEQ ID NO: 198, a homolog or an ortholog of a different organism species, and the like. The hsa-miR-24-3p gene can be obtained by a method described in Lagos-Quintana M et al., 2001, Science, Vol. 294, p. 853-858. Also, “hsa-mir-24-1 and hsa-mir-24-2” (miRBase Accession Nos. MI0000080 and MI0000081, SEQ ID NOs: 535 and 536) having a hairpin-like structure are known as precursors of “hsa-miR-24-3p”.

The term “hsa-miR-296-3p gene” or “hsa-miR-296-3p” used herein includes the hsa-miR-296-3p gene (miRBase Accession No. MIMAT0004679) described in SEQ ID NO: 199, a homolog or an ortholog of a different organism species, and the like. The hsa-miR-296-3p gene can be obtained by a method described in Houbaviy H B et al., 2003, Dev Cell, Vol. 5, p. 351-358. Also, “hsa-mir-296” (miRBase Accession No. MI0000747, SEQ ID NO: 537) having a hairpin-like structure is known as a precursor of “hsa-miR-296-3p”.

The term “hsa-miR-296-5p gene” or “hsa-miR-296-5p” used herein includes the hsa-miR-296-5p gene (miRBase Accession No. MIMAT0000690) described in SEQ ID NO: 200, a homolog or an ortholog of a different organism species, and the like. The hsa-miR-296-5p gene can be obtained by a method described in Houbaviy H B et al., 2003, Dev Cell, Vol. 5, p. 351-358. Also, “hsa-mir-296” (miRBase Accession No. MI0000747, SEQ ID NO: 537) having a hairpin-like structure is known as a precursor of “hsa-miR-296-5p”.

The term “hsa-miR-3131 gene” or “hsa-miR-3131” used herein includes the hsa-miR-3131 gene (miRBase Accession No. MIMAT0014996) described in SEQ ID NO: 201, a homolog or an ortholog of a different organism species, and the like. The hsa-miR-3131 gene can be obtained by a method described in Stark M S et al., 2010, PLoS One, Vol. 5, e9685. Also, “hsa-mir-3131” (miRBase Accession No. MI0014151, SEQ ID NO: 538) having a hairpin-like structure is known as a precursor of “hsa-miR-3131”.

The term “hsa-miR-3162-5p gene” or “hsa-miR-3162-5p” used herein includes the hsa-miR-3162-5p gene (miRBase Accession No. MIMAT0015036) described in SEQ ID NO: 202, a homolog or an ortholog of a different organism species, and the like. The hsa-miR-3162-5p gene can be obtained by a method described in Stark M S et al., 2010, PLoS One, Vol. 5, e9685. Also, “hsa-mir-3162” (miRBase Accession No. MI0014192, SEQ ID NO: 539) having a hairpin-like structure is known as a precursor of “hsa-miR-3162-5p”.

The term “hsa-miR-3188 gene” or “hsa-miR-3188” used herein includes the hsa-miR-3188 gene (miRBase Accession No. MIMAT0015070) described in SEQ ID NO: 203, a homolog or an ortholog of a different organism species, and the like. The hsa-miR-3188 gene can be obtained by a method described in Stark M S et al., 2010, PLoS One, Vol. 5, e9685. Also, “hsa-mir-3188” (miRBase Accession No. MI0014232, SEQ ID NO: 540) having a hairpin-like structure is known as a precursor of “hsa-miR-3188”.

The term “hsa-miR-3196 gene” or “hsa-miR-3196” used herein includes the hsa-miR-3196 gene (miRBase Accession No. MIMAT0015080) described in SEQ ID NO: 204, a homolog or an ortholog of a different organism species, and the like. The hsa-miR-3196 gene can be obtained by a method described in Stark M S et al., 2010, PLoS One, Vol. 5, e9685. Also, “hsa-mir-3196” (miRBase Accession No. MI0014241, SEQ ID NO: 541) having a hairpin-like structure is known as a precursor of “hsa-miR-3196”.

The term “hsa-miR-3197 gene” or “hsa-miR-3197” used herein includes the hsa-miR-3197 gene (miRBase Accession No. MIMAT0015082) described in SEQ ID NO: 205, a homolog or an ortholog of a different organism species, and the like. The hsa-miR-3197 gene can be obtained by a method described in Stark M S et al., 2010, PLoS One, Vol. 5, e9685. Also, “hsa-mir-3197” (miRBase Accession No. MI0014245, SEQ ID NO: 542) having a hairpin-like structure is known as a precursor of “hsa-miR-3197”.

The term “hsa-miR-320a gene” or “hsa-miR-320a” used herein includes the hsa-miR-320a gene (miRBase Accession No. MIMAT0000510) described in SEQ ID NO: 206, a homolog or an ortholog of a different organism species, and the like. The hsa-miR-320a gene can be obtained by a method described in Michael M Z et al., 2003, Mol Cancer Res, Vol. 1, p. 882-891. Also, “hsa-mir-320a” (miRBase Accession No. MI0000542, SEQ ID NO: 543) having a hairpin-like structure is known as a precursor of “hsa-miR-320a”.

The term “hsa-miR-342-5p gene” or “hsa-miR-342-5p” used herein includes the hsa-miR-342-5p gene (miRBase Accession No. MIMAT0004694) described in SEQ ID NO: 207, a homolog or an ortholog of a different organism species, and the like. The hsa-miR-342-5p gene can be obtained by a method described in Kim J et al., 2004, Proc Natl Acad Sci USA, Vol. 101, p. 360-365. Also, “hsa-mir-342” (miRBase Accession No. MI0000805, SEQ ID NO: 544) having a hairpin-like structure is known as a precursor of “hsa-miR-342-5p”.

The term “hsa-miR-3621 gene” or “hsa-miR-3621” used herein includes the hsa-miR-3621 gene (miRBase Accession No. MIMAT0018002) described in SEQ ID NO: 208, a homolog or an ortholog of a different organism species, and the like. The hsa-miR-3621 gene can be obtained by a method described in Witten D et al., 2010, BMC Biol, Vol. 8, p. 58. Also, “hsa-mir-3621” (miRBase Accession No. MI0016012, SEQ ID NO: 545) having a hairpin-like structure is known as a precursor of “hsa-miR-3621”.

The term “hsa-miR-3648 gene” or “hsa-miR-3648” used herein includes the hsa-miR-3648 gene (miRBase Accession No. MIMAT0018068) described in SEQ ID NO: 209, a homolog or an ortholog of a different organism species, and the like. The hsa-miR-3648 gene can be obtained by a method described in Meiri E et al., 2010, Nucleic Acids Res, Vol. 38, p. 6234-6246. Also, “hsa-mir-3648” (miRBase Accession No. MI0016048, SEQ ID NO: 546) having a hairpin-like structure is known as a precursor of “hsa-miR-3648”.

The term “hsa-miR-3656 gene” or “hsa-miR-3656” used herein includes the hsa-miR-3656 gene (miRBase Accession No. MIMAT0018076) described in SEQ ID NO: 210, a homolog or an ortholog of a different organism species, and the like. The hsa-miR-3656 gene can be obtained by a method described in Meiri E et al., 2010, Nucleic Acids Res, Vol. 38, p. 6234-6246. Also, “hsa-mir-3656” (miRBase Accession No. MI0016056, SEQ ID NO: 547) having a hairpin-like structure is known as a precursor of “hsa-miR-3656”.

The term “hsa-miR-365a-5p gene” or “hsa-miR-365a-5p” used herein includes the hsa-miR-365a-5p gene (miRBase Accession No. MIMAT0009199) described in SEQ ID NO: 211, a homolog or an ortholog of a different organism species, and the like. The hsa-miR-365a-5p gene can be obtained by a method described in Xie X et al., 2005, Nature, Vol. 434, p. 338-345. Also, “hsa-mir-365a” (miRBase Accession No. MI0000767, SEQ ID NO: 548) having a hairpin-like structure is known as a precursor of “hsa-miR-365a-5p”.

The term “hsa-miR-3665 gene” or “hsa-miR-3665” used herein includes the hsa-miR-3665 gene (miRBase Accession No. MIMAT0018087) described in SEQ ID NO: 212, a homolog or an ortholog of a different organism species, and the like. The hsa-miR-3665 gene can be obtained by a method described in ‘Xie X et al., 2005, Nature, Vol. 434, p. 338-345′. Also, “hsa-mir-3665” (miRBase Accession No. MI0016066, SEQ ID NO: 549) having a hairpin-like structure is known as a precursor of “hsa-miR-3665”.

The term “hsa-miR-3679-5p gene” or “hsa-miR-3679-5p” used herein includes the hsa-miR-3679-5p gene (miRBase Accession No. MIMAT0018104) described in SEQ ID NO: 213, a homolog or an ortholog of a different organism species, and the like. The hsa-miR-3679-5p gene can be obtained by a method described in Creighton C J et al., 2010, PLoS One, Vol. 5, e9637. Also, “hsa-mir-3679” (miRBase Accession No. MI0016080, SEQ ID NO: 550) having a hairpin-like structure is known as a precursor of “hsa-miR-3679-5p”.

The term “hsa-miR-371a-5p gene” or “hsa-miR-371a-5p” used herein includes the hsa-miR-371a-5p gene (miRBase Accession No. MIMAT0004687) described in SEQ ID NO: 214, a homolog or an ortholog of a different organism species, and the like. The hsa-miR-371a-5p gene can be obtained by a method described in Suh M R et al., 2004, Dev Biol, Vol. 270, p. 488-498. Also, “hsa-mir-371a” (miRBase Accession No. MI0000779, SEQ ID NO: 551) having a hairpin-like structure is known as a precursor of “hsa-miR-371a-5p”.

The term “hsa-miR-3940-5p gene” or “hsa-miR-3940-5p” used herein includes the hsa-miR-3940-5p gene (miRBase Accession No. MIMAT0019229) described in SEQ ID NO: 215, a homolog or an ortholog of a different organism species, and the like. The hsa-miR-3940-5p gene can be obtained by a method described in Liao J Y et al., 2010, PLoS One, Vol. 5, e10563. Also, “hsa-mir-3940” (miRBase Accession No. MI0016597, SEQ ID NO: 552) having a hairpin-like structure is known as a precursor of “hsa-miR-3940-5p”.

The term “hsa-miR-423-5p gene” or “hsa-miR-423-5p” used herein includes the hsa-miR-423-5p gene (miRBase Accession No. MIMAT0004748) described in SEQ ID NO: 216, a homolog or an ortholog of a different organism species, and the like. The hsa-miR-423-5p gene can be obtained by a method described in Kasashima K et al., 2004, Biochem Biophys Res Commun, Vol. 322, p. 403-410. Also, “hsa-mir-423” (miRBase Accession No. MI0001445, SEQ ID NO: 553) having a hairpin-like structure is known as a precursor of “hsa-miR-423-5p”.

The term “hsa-miR-4257 gene” or “hsa-miR-4257” used herein includes the hsa-miR-4257 gene (miRBase Accession No. MIMAT0016878) described in SEQ ID NO: 217, a homolog or an ortholog of a different organism species, and the like. The hsa-miR-4257 gene can be obtained by a method described in Goff L A et al., 2009, PLoS One, Vol. 4, e7192. Also, “hsa-mir-4257” (miRBase Accession No. MI0015856, SEQ ID NO: 554) having a hairpin-like structure is known as a precursor of “hsa-miR-4257”.

The term “hsa-miR-4270 gene” or “hsa-miR-4270” used herein includes the hsa-miR-4270 gene (miRBase Accession No. MIMAT0016900) described in SEQ ID NO: 218, a homolog or an ortholog of a different organism species, and the like. The hsa-miR-4270 gene can be obtained by a method described in Goff L A et al., 2009, PLoS One, Vol. 4, e7192. Also, “hsa-mir-4270” (miRBase Accession No. MI0015878, SEQ ID NO: 555) having a hairpin-like structure is known as a precursor of “hsa-miR-4270”.

The term “hsa-miR-4271 gene” or “hsa-miR-4271” used herein includes the hsa-miR-4271 gene (miRBase Accession No. MIMAT0016901) described in SEQ ID NO: 219, a homolog or an ortholog of a different organism species, and the like. The hsa-miR-4271 gene can be obtained by a method described in Goff L A et al., 2009, PLoS One, Vol. 4, e7192. Also, “hsa-mir-4271” (miRBase Accession No. MI0015879, SEQ ID NO: 556) having a hairpin-like structure is known as a precursor of “hsa-miR-4271”.

The term “hsa-miR-4286 gene” or “hsa-miR-4286” used herein includes the hsa-miR-4286 gene (miRBase Accession No. MIMAT0016916) described in SEQ ID NO: 220, a homolog or an ortholog of a different organism species, and the like. The hsa-miR-4286 gene can be obtained by a method described in Goff L A et al., 2009, PLoS One, Vol. 4, e7192. Also, “hsa-mir-4286” (miRBase Accession No. MI0015894, SEQ ID NO: 557) having a hairpin-like structure is known as a precursor of “hsa-miR-4286”.

The term “hsa-miR-4298 gene” or “hsa-miR-4298” used herein includes the hsa-miR-4298 gene (miRBase Accession No. MIMAT0016852) described in SEQ ID NO: 221, a homolog or an ortholog of a different organism species, and the like. The hsa-miR-4298 gene can be obtained by a method described in Goff L A et al., 2009, PLoS One, Vol. 4, e7192. Also, “hsa-mir-4298” (miRBase Accession No. MI0015830, SEQ ID NO: 558) having a hairpin-like structure is known as a precursor of “hsa-miR-4298”.

The term “hsa-miR-4417 gene” or “hsa-miR-4417” used herein includes the hsa-miR-4417 gene (miRBase Accession No. MIMAT0018929) described in SEQ ID NO: 222, a homolog or an ortholog of a different organism species, and the like. The hsa-miR-4417 gene can be obtained by a method described in Jima D D et al., 2010, Blood, Vol. 116, e118-e127. Also, “hsa-mir-4417” (miRBase Accession No. MI0016753, SEQ ID NO: 559) having a hairpin-like structure is known as a precursor of “hsa-miR-4417”.

The term “hsa-miR-4442 gene” or “hsa-miR-4442” used herein includes the hsa-miR-4442 gene (miRBase Accession No. MIMAT0018960) described in SEQ ID NO: 223, a homolog or an ortholog of a different organism species, and the like. The hsa-miR-4442 gene can be obtained by a method described in Jima D D et al., 2010, Blood, Vol. 116, e118-e127. Also, “hsa-mir-4442” (miRBase Accession No. MI0016785, SEQ ID NO: 560) having a hairpin-like structure is known as a precursor of “hsa-miR-4442”.

The term “hsa-miR-4446-3p gene” or “hsa-miR-4446-3p” used herein includes the hsa-miR-4446-3p gene (miRBase Accession No. MIMAT0018965) described in SEQ ID NO: 224, a homolog or an ortholog of a different organism species, and the like. The hsa-miR-4446-3p gene can be obtained by a method described in Jima D D et al., 2010, Blood, Vol. 116, e118-e127. Also, “hsa-mir-4446” (miRBase Accession No. MI0016789, SEQ ID NO: 561) having a hairpin-like structure is known as a precursor of “hsa-miR-4446-3p”.

The term “hsa-miR-4448 gene” or “hsa-miR-4448” used herein includes the hsa-miR-4448 gene (miRBase Accession No. MIMAT0018967) described in SEQ ID NO: 225, a homolog or an ortholog of a different organism species, and the like. The hsa-miR-4448 gene can be obtained by a method described in Jima D D et al., 2010, Blood, Vol. 116, e118-e127. Also, “hsa-mir-4448” (miRBase Accession No. MI0016791, SEQ ID NO: 562) having a hairpin-like structure is known as a precursor of “hsa-miR-4448”.

The term “hsa-miR-4454 gene” or “hsa-miR-4454” used herein includes the hsa-miR-4454 gene (miRBase Accession No. MIMAT0018976) described in SEQ ID NO: 226, a homolog or an ortholog of a different organism species, and the like. The hsa-miR-4454 gene can be obtained by a method described in Jima D D et al., 2010, Blood, Vol. 116, e118-e127. Also, “hsa-mir-4454” (miRBase Accession No. MI0016800, SEQ ID NO: 563) having a hairpin-like structure is known as a precursor of “hsa-miR-4454”.

The term “hsa-miR-4467 gene” or “hsa-miR-4467” used herein includes the hsa-miR-4467 gene (miRBase Accession No. MIMAT0018994) described in SEQ ID NO: 227, a homolog or an ortholog of a different organism species, and the like. The hsa-miR-4467 gene can be obtained by a method described in Jima D D et al., 2010, Blood, Vol. 116, e118-e127. Also, “hsa-mir-4467” (miRBase Accession No. MI0016818, SEQ ID NO: 564) having a hairpin-like structure is known as a precursor of “hsa-miR-4467”.

The term “hsa-miR-4472 gene” or “hsa-miR-4472” used herein includes the hsa-miR-4472 gene (miRBase Accession No. MIMAT0018999) described in SEQ ID NO: 228, a homolog or an ortholog of a different organism species, and the like. The hsa-miR-4472 gene can be obtained by a method described in Jima D D et al., 2010, Blood, Vol. 116, e118-e127. Also, “hsa-mir-4472-1 and hsa-mir-4472-2” (miRBase Accession Nos. MI0016823 and MI0016824, SEQ ID NOs: 565 and 566) having a hairpin-like structure are known as precursors of “hsa-miR-4472”.

The term “hsa-miR-4507 gene” or “hsa-miR-4507” used herein includes the hsa-miR-4507 gene (miRBase Accession No. MIMAT0019044) described in SEQ ID NO: 229, a homolog or an ortholog of a different organism species, and the like. The hsa-miR-4507 gene can be obtained by a method described in Jima D D et al., 2010, Blood, Vol. 116, e118-e127. Also, “hsa-mir-4507” (miRBase Accession No. MI0016871, SEQ ID NO: 567) having a hairpin-like structure is known as a precursor of “hsa-miR-4507”.

The term “hsa-miR-4516 gene” or “hsa-miR-4516” used herein includes the hsa-miR-4516 gene (miRBase Accession No. MIMAT0019053) described in SEQ ID NO: 230, a homolog or an ortholog of a different organism species, and the like. The hsa-miR-4516 gene can be obtained by a method described in Jima D D et al., 2010, Blood, Vol. 116, e118-e127. Also, “hsa-mir-4516” (miRBase Accession No. MI0016882, SEQ ID NO: 568) having a hairpin-like structure is known as a precursor of “hsa-miR-4516”.

The term “hsa-miR-451a gene” or “hsa-miR-451a” used herein includes the hsa-miR-451a gene (miRBase Accession No. MIMAT0001631) described in SEQ ID NO: 231, a homolog or an ortholog of a different organism species, and the like. The hsa-miR-451a gene can be obtained by a method described in Altuvia Y et al., 2005, Nucleic Acids Res, Vol. 33, p. 2697-2706. Also, “hsa-mir-451a” (miRBase Accession No. MI0001729, SEQ ID NO: 569) having a hairpin-like structure is known as a precursor of “hsa-miR-451a”.

The term “hsa-miR-4649-5p gene” or “hsa-miR-4649-5p” used herein includes the hsa-miR-4649-5p gene (miRBase Accession No. MIMAT0019711) described in SEQ ID NO: 232, a homolog or an ortholog of a different organism species, and the like. The hsa-miR-4649-5p gene can be obtained by a method described in Persson H et al., 2011, Cancer Res, Vol. 71, p. 78-86. Also, “hsa-mir-4649” (miRBase Accession No. MI0017276, SEQ ID NO: 570) having a hairpin-like structure is known as a precursor of “hsa-miR-4649-5p”.

The term “hsa-miR-4651 gene” or “hsa-miR-4651” used herein includes the hsa-miR-4651 gene (miRBase Accession No. MIMAT0019715) described in SEQ ID NO: 233, a homolog or an ortholog of a different organism species, and the like. The hsa-miR-4651 gene can be obtained by a method described in Persson H et al., 2011, Cancer Res, Vol. 71, p. 78-86. Also, “hsa-mir-4651” (miRBase Accession No. MI0017279, SEQ ID NO: 571) having a hairpin-like structure is known as a precursor of “hsa-miR-4651”.

The term “hsa-miR-4665-3p gene” or “hsa-miR-4665-3p” used herein includes the hsa-miR-4665-3p gene (miRBase Accession No. MIMAT0019740) described in SEQ ID NO: 234, a homolog or an ortholog of a different organism species, and the like. The hsa-miR-4665-3p gene can be obtained by a method described in Persson H et al., 2011, Cancer Res, Vol. 71, p. 78-86. Also, “hsa-mir-4665” (miRBase Accession No. MI0017295, SEQ ID NO: 401) having a hairpin-like structure is known as a precursor of “hsa-miR-4665-3p”.

The term “hsa-miR-4674 gene” or “hsa-miR-4674” used herein includes the hsa-miR-4674 gene (miRBase Accession No. MIMAT0019756) described in SEQ ID NO: 235, a homolog or an ortholog of a different organism species, and the like. The hsa-miR-4674 gene can be obtained by a method described in Persson H et al., 2011, Cancer Res, Vol. 71, p. 78-86. Also, “hsa-mir-4674” (miRBase Accession No. MI0017305, SEQ ID NO: 572) having a hairpin-like structure is known as a precursor of “hsa-miR-4674”.

The term “hsa-miR-4675 gene” or “hsa-miR-4675” used herein includes the hsa-miR-4675 gene (miRBase Accession No. MIMAT0019757) described in SEQ ID NO: 236, a homolog or an ortholog of a different organism species, and the like. The hsa-miR-4675 gene can be obtained by a method described in Persson H et al., 2011, Cancer Res, Vol. 71, p. 78-86. Also, “hsa-mir-4675” (miRBase Accession No. MI0017306, SEQ ID NO: 573) having a hairpin-like structure is known as a precursor of “hsa-miR-4675”.

The term “hsa-miR-4689 gene” or “hsa-miR-4689” used herein includes the hsa-miR-4689 gene (miRBase Accession No. MIMAT0019778) described in SEQ ID NO: 237, a homolog or an ortholog of a different organism species, and the like. The hsa-miR-4689 gene can be obtained by a method described in Persson H et al., 2011, Cancer Res, Vol. 71, p. 78-86. Also, “hsa-mir-4689” (miRBase Accession No. MI0017322, SEQ ID NO: 574) having a hairpin-like structure is known as a precursor of “hsa-miR-4689”.

The term “hsa-miR-4695-5p gene” or “hsa-miR-4695-5p” used herein includes the hsa-miR-4695-5p gene (miRBase Accession No. MIMAT0019788) described in SEQ ID NO: 238, a homolog or an ortholog of a different organism species, and the like. The hsa-miR-4695-5p gene can be obtained by a method described in Persson H et al., 2011, Cancer Res, Vol. 71, p. 78-86. Also, “hsa-mir-4695” (miRBase Accession No. MI0017328, SEQ ID NO: 575) having a hairpin-like structure is known as a precursor of “hsa-miR-4695-5p”.

The term “hsa-miR-4697-5p gene” or “hsa-miR-4697-5p” used herein includes the hsa-miR-4697-5p gene (miRBase Accession No. MIMAT0019791) described in SEQ ID NO: 239, a homolog or an ortholog of a different organism species, and the like. The hsa-miR-4697-5p gene can be obtained by a method described in Persson H et al., 2011, Cancer Res, Vol. 71, p. 78-86. Also, “hsa-mir-4697” (miRBase Accession No. MI0017330, SEQ ID NO: 576) having a hairpin-like structure is known as a precursor of “hsa-miR-4697-5p”.

The term “hsa-miR-4725-3p gene” or “hsa-miR-4725-3p” used herein includes the hsa-miR-4725-3p gene (miRBase Accession No. MIMAT0019844) described in SEQ ID NO: 240, a homolog or an ortholog of a different organism species, and the like. The hsa-miR-4725-3p gene can be obtained by a method described in Persson H et al., 2011, Cancer Res, Vol. 71, p. 78-86. Also, “hsa-mir-4725” (miRBase Accession No. MI0017362, SEQ ID NO: 577) having a hairpin-like structure is known as a precursor of “hsa-miR-4725-3p”.

The term “hsa-miR-4739 gene” or “hsa-miR-4739” used herein includes the hsa-miR-4739 gene (miRBase Accession No. MIMAT0019868) described in SEQ ID NO: 241, a homolog or an ortholog of a different organism species, and the like. The hsa-miR-4739 gene can be obtained by a method described in Persson H et al., 2011, Cancer Res, Vol. 71, p. 78-86. Also, “hsa-mir-4739” (miRBase Accession No. MI0017377, SEQ ID NO: 578) having a hairpin-like structure is known as a precursor of “hsa-miR-4739”.

The term “hsa-miR-4745-5p gene” or “hsa-miR-4745-5p” used herein includes the hsa-miR-4745-5p gene (miRBase Accession No. MIMAT0019878) described in SEQ ID NO: 242, a homolog or an ortholog of a different organism species, and the like. The hsa-miR-4745-5p gene can be obtained by a method described in Persson H et al., 2011, Cancer Res, Vol. 71, p. 78-86. Also, “hsa-mir-4745” (miRBase Accession No. MI0017384, SEQ ID NO: 579) having a hairpin-like structure is known as a precursor of “hsa-miR-4745-5p”.

The term “hsa-miR-4763-3p gene” or “hsa-miR-4763-3p” used herein includes the hsa-miR-4763-3p gene (miRBase Accession No. MIMAT0019913) described in SEQ ID NO: 243, a homolog or an ortholog of a different organism species, and the like. The hsa-miR-4763-3p gene can be obtained by a method described in Persson H et al., 2011, Cancer Res, Vol. 71, p. 78-86. Also, “hsa-mir-4763” (miRBase Accession No. MI0017404, SEQ ID NO: 415) having a hairpin-like structure is known as a precursor of “hsa-miR-4763-3p”.

The term “hsa-miR-4792 gene” or “hsa-miR-4792” used herein includes the hsa-miR-4792 gene (miRBase Accession No. MIMAT0019964) described in SEQ ID NO: 244, a homolog or an ortholog of a different organism species, and the like. The hsa-miR-4792 gene can be obtained by a method described in Persson H et al., 2011, Cancer Res, Vol. 71, p. 78-86. Also, “hsa-mir-4792” (miRBase Accession No. MI0017439, SEQ ID NO: 580) having a hairpin-like structure is known as a precursor of “hsa-miR-4792”.

The term “hsa-miR-486-3p gene” or “hsa-miR-486-3p” used herein includes the hsa-miR-486-3p gene (miRBase Accession No. MIMAT0004762) described in SEQ ID NO: 245, a homolog or an ortholog of a different organism species, and the like. The hsa-miR-486-3p gene can be obtained by a method described in Fu H et al., 2005, FEBS Lett, Vol. 579, p. 3849-3854. Also, “hsa-mir-486 and hsa-mir-486-2” (miRBase Accession Nos. MI0002470 and MI0023622, SEQ ID NOs: 581 and 582) having a hairpin-like structure are known as precursors of “hsa-miR-486-3p”.

The term “hsa-miR-5001-5p gene” or “hsa-miR-5001-5p” used herein includes the hsa-miR-5001-5p gene (miRBase Accession No. MIMAT0021021) described in SEQ ID NO: 246, a homolog or an ortholog of a different organism species, and the like. The hsa-miR-5001-5p gene can be obtained by a method described in Hansen T B et al., 2011, RNA Biol, Vol. 8, p. 378-383. Also, “hsa-mir-5001” (miRBase Accession No. MI0017867, SEQ ID NO: 583) having a hairpin-like structure is known as a precursor of “hsa-miR-5001-5p”.

The term “hsa-miR-5195-3p gene” or “hsa-miR-5195-3p” used herein includes the hsa-miR-5195-3p gene (miRBase Accession No. MIMAT0021127) described in SEQ ID NO: 247, a homolog or an ortholog of a different organism species, and the like. The hsa-miR-5195-3p gene can be obtained by a method described in Schotte D et al., 2011, Leukemia, Vol. 25, p. 1389-1399. Also, “hsa-mir-5195” (miRBase Accession No. MI0018174, SEQ ID NO: 584) having a hairpin-like structure is known as a precursor of “hsa-miR-5195-3p”.

The term “hsa-miR-550a-5p gene” or “hsa-miR-550a-5p” used herein includes the hsa-miR-550a-5p gene (miRBase Accession No. MIMAT0004800) described in SEQ ID NO: 248, a homolog or an ortholog of a different organism species, and the like. The hsa-miR-550a-5p gene can be obtained by a method described in Cummins J M, 2006, Proc Natl Acad Sci, Vol. 103, p. 3687-3692. Also, “hsa-mir-550a-1 and hsa-mir-550a-2” (miRBase Accession Nos. MI0003600 and MI0003601, SEQ ID NOs: 585 and 586) having a hairpin-like structure are known as precursors of “hsa-miR-550a-5p”.

The term “hsa-miR-5698 gene” or “hsa-miR-5698” used herein includes the hsa-miR-5698 gene (miRBase Accession No. MIMAT0022491) described in SEQ ID NO: 249, a homolog or an ortholog of a different organism species, and the like. The hsa-miR-5698 gene can be obtained by a method described in Watahiki A et al., 2011, PLoS One, Vol. 6, e24950. Also, “hsa-mir-5698” (miRBase Accession No. MI0019305, SEQ ID NO: 587) having a hairpin-like structure is known as a precursor of “hsa-miR-5698”.

The term “hsa-miR-6075 gene” or “hsa-miR-6075” used herein includes the hsa-miR-6075 gene (miRBase Accession No. MIMAT0023700) described in SEQ ID NO: 250, a homolog or an ortholog of a different organism species, and the like. The hsa-miR-6075 gene can be obtained by a method described in Voellenkle C et al., 2012, RNA, Vol. 18, p. 472-484. Also, “hsa-mir-6075” (miRBase Accession No. MI0020352, SEQ ID NO: 588) having a hairpin-like structure is known as a precursor of “hsa-miR-6075”.

The term “hsa-miR-6088 gene” or “hsa-miR-6088” used herein includes the hsa-miR-6088 gene (miRBase Accession No. MIMAT0023713) described in SEQ ID NO: 251, a homolog or an ortholog of a different organism species, and the like. The hsa-miR-6088 gene can be obtained by a method described in Yoo J K et al., 2012, Stem Cells Dev, Vol. 21, p. 2049-2057. Also, “hsa-mir-6088” (miRBase Accession No. MI0020365, SEQ ID NO: 589) having a hairpin-like structure is known as a precursor of “hsa-miR-6088”.

The term “hsa-miR-6089 gene” or “hsa-miR-6089” used herein includes the hsa-miR-6089 gene (miRBase Accession No. MIMAT0023714) described in SEQ ID NO: 252, a homolog or an ortholog of a different organism species, and the like. The hsa-miR-6089 gene can be obtained by a method described in Yoo J K et al., 2012, Stem Cells Dev, Vol. 21, p. 2049-2057. Also, “hsa-mir-6089-1 and hsa-mir-6089-2” (miRBase Accession Nos. MI0020366 and MI0023563, SEQ ID NOs: 590 and 591) having a hairpin-like structure are known as precursors of “hsa-miR-6089”.

The term “hsa-miR-6125 gene” or “hsa-miR-6125” used herein includes the hsa-miR-6125 gene (miRBase Accession No. MIMAT0024598) described in SEQ ID NO: 253, a homolog or an ortholog of a different organism species, and the like. The hsa-miR-6125 gene can be obtained by a method described in Smith J L et al., 2012, J Virol, Vol. 86, p. 5278-5287. Also, “hsa-mir-6125” (miRBase Accession No. MI0021259, SEQ ID NO: 592) having a hairpin-like structure is known as a precursor of “hsa-miR-6125”.

The term “hsa-miR-6126 gene” or “hsa-miR-6126” used herein includes the hsa-miR-6126 gene (miRBase Accession No. MIMAT0024599) described in SEQ ID NO: 254, a homolog or an ortholog of a different organism species, and the like. The hsa-miR-6126 gene can be obtained by a method described in Smith J L et al., 2012, J Virol, Vol. 86, p. 5278-5287. Also, “hsa-mir-6126” (miRBase Accession No. MI0021260, SEQ ID NO: 593) having a hairpin-like structure is known as a precursor of “hsa-miR-6126”.

The term “hsa-miR-614 gene” or “hsa-miR-614” used herein includes the hsa-miR-614 gene (miRBase Accession No. MIMAT0003282) described in SEQ ID NO: 255, a homolog or an ortholog of a different organism species, and the like. The hsa-miR-614 gene can be obtained by a method described in Cummins J M et al., 2006, Proc Natl Acad Sci USA, Vol. 103, p. 3687-3692. Also, “hsa-mir-614” (miRBase Accession No. M10003627, SEQ ID NO: 594) having a hairpin-like structure is known as a precursor of “hsa-miR-614”.

The term “hsa-miR-615-5p gene” or “hsa-miR-615-5p” used herein includes the hsa-miR-615-5p gene (miRBase Accession No. MIMAT0004804) described in SEQ ID NO: 256, a homolog or an ortholog of a different organism species, and the like. The hsa-miR-615-5p gene can be obtained by a method described in Cummins J M et al., 2006, Proc Natl Acad Sci USA, Vol. 103, p. 3687-3692. Also, “hsa-mir-615” (miRBase Accession No. MI0003628, SEQ ID NO: 595) having a hairpin-like structure is known as a precursor of “hsa-miR-615-5p”.

The term “hsa-miR-619-5p gene” or “hsa-miR-619-5p” used herein includes the hsa-miR-619-5p gene (miRBase Accession No. MIMAT0026622) described in SEQ ID NO: 257, a homolog or an ortholog of a different organism species, and the like. The hsa-miR-619-5p gene can be obtained by a method described in Cummins J M et al., 2006, Proc Natl Acad Sci USA, Vol. 103, p. 3687-3692. Also, “hsa-mir-619” (miRBase Accession No. MI0003633, SEQ ID NO: 596) having a hairpin-like structure is known as a precursor of “hsa-miR-619-5p”.

The term “hsa-miR-638 gene” or “hsa-miR-638” used herein includes the hsa-miR-638 gene (miRBase Accession No. MIMAT0003308) described in SEQ ID NO: 258, a homolog or an ortholog of a different organism species, and the like. The hsa-miR-638 gene can be obtained by a method described in Cummins J M et al., 2006, Proc Natl Acad Sci USA, Vol. 103, p. 3687-3692. Also, “hsa-mir-638” (miRBase Accession No. MI0003653, SEQ ID NO: 597) having a hairpin-like structure is known as a precursor of “hsa-miR-638”.

The term “hsa-miR-642b-3p gene” or “hsa-miR-642b-3p” used herein includes the hsa-miR-642b-3p gene (miRBase Accession No. MIMAT0018444) described in SEQ ID NO: 259, a homolog or an ortholog of a different organism species, and the like. The hsa-miR-642b-3p gene can be obtained by a method described in Witten D et al., 2010, BMC Biol, Vol. 8, p. 58. Also, “hsa-mir-642b” (miRBase Accession No. MI0016685, SEQ ID NO: 598) having a hairpin-like structure is known as a precursor of “hsa-miR-642b-3p”.

The term “hsa-miR-650 gene” or “hsa-miR-650” used herein includes the hsa-miR-650 gene (miRBase Accession No. MIMAT0003320) described in SEQ ID NO: 260, a homolog or an ortholog of a different organism species, and the like. The hsa-miR-650 gene can be obtained by a method described in Cummins J M et al., 2006, Proc Natl Acad Sci USA., Vol. 103, 3687-3692. Also, “hsa-mir-650” (miRBase Accession No. MI0003665, SEQ ID NO: 599) having a hairpin-like structure is known as a precursor of “hsa-miR-650”.

The term “hsa-miR-663a gene” or “hsa-miR-663a” used herein includes the hsa-miR-663a gene (miRBase Accession No. MIMAT0003326) described in SEQ ID NO: 261, a homolog or an ortholog of a different organism species, and the like. The hsa-miR-663a gene can be obtained by a method described in Cummins J M et al., 2006, Proc Natl Acad Sci USA, Vol. 103, p. 3687-3692. Also, “hsa-mir-663a” (miRBase Accession No. MI0003672, SEQ ID NO: 600) having a hairpin-like structure is known as a precursor of “hsa-miR-663a”.

The term “hsa-miR-663b gene” or “hsa-miR-663b” used herein includes the hsa-miR-663b gene (miRBase Accession No. MIMAT0005867) described in SEQ ID NO: 262, a homolog or an ortholog of a different organism species, and the like. The hsa-miR-663b gene can be obtained by a method described in Takada S et al., 2008, Leukemia, Vol. 22, p. 1274-1278. Also, “hsa-mir-663b” (miRBase Accession No. MI0006336, SEQ ID NO: 601) having a hairpin-like structure is known as a precursor of “hsa-miR-663b”.

The term “hsa-miR-6717-5p gene” or “hsa-miR-6717-5p” used herein includes the hsa-miR-6717-5p gene (miRBase Accession No. MIMAT0025846) described in SEQ ID NO: 263, a homolog or an ortholog of a different organism species, and the like. The hsa-miR-6717-5p gene can be obtained by a method described in Li Y et al., 2012, Gene, Vol. 497, p. 330-335. Also, “hsa-mir-6717” (miRBase Accession No. MI0022551, SEQ ID NO: 602) having a hairpin-like structure is known as a precursor of “hsa-miR-6717-5p”.

The term “hsa-miR-6721-5p gene” or “hsa-miR-6721-5p” used herein includes the hsa-miR-6721-5p gene (miRBase Accession No. MIMAT0025852) described in SEQ ID NO: 264, a homolog or an ortholog of a different organism species, and the like. The hsa-miR-6721-5p gene can be obtained by a method described in Li Y et al., 2012, Gene, Vol. 497, p. 330-335. Also, “hsa-mir-6721” (miRBase Accession No. MI0022556, SEQ ID NO: 603) having a hairpin-like structure is known as a precursor of “hsa-miR-6721-5p”.

The term “hsa-miR-6726-5p gene” or “hsa-miR-6726-5p” used herein includes the hsa-miR-6726-5p gene (miRBase Accession No. MIMAT0027353) described in SEQ ID NO: 265, a homolog or an ortholog of a different organism species, and the like. The hsa-miR-6726-5p gene can be obtained by a method described in Ladewig E et al., 2012, Genome Res, Vol. 22, p. 1634-1645. Also, “hsa-mir-6726” (miRBase Accession No. MI0022571, SEQ ID NO: 604) having a hairpin-like structure is known as a precursor of “hsa-miR-6726-5p”.

The term “hsa-miR-6727-5p gene” or “hsa-miR-6727-5p” used herein includes the hsa-miR-6727-5p gene (miRBase Accession No. MIMAT0027355) described in SEQ ID NO: 266, a homolog or an ortholog of a different organism species, and the like. The hsa-miR-6727-5p gene can be obtained by a method described in Ladewig E et al., 2012, Genome Res, Vol. 22, p. 1634-1645. Also, “hsa-mir-6727” (miRBase Accession No. MI0022572, SEQ ID NO: 605) having a hairpin-like structure is known as a precursor of “hsa-miR-6727-5p”.

The term “hsa-miR-6738-5p gene” or “hsa-miR-6738-5p” used herein includes the hsa-miR-6738-5p gene (miRBase Accession No. MIMAT0027377) described in SEQ ID NO: 267, a homolog or an ortholog of a different organism species, and the like. The hsa-miR-6738-5p gene can be obtained by a method described in Ladewig E et al., 2012, Genome Res, Vol. 22, p. 1634-1645. Also, “hsa-mir-6738” (miRBase Accession No. MI0022583, SEQ ID NO: 606) having a hairpin-like structure is known as a precursor of “hsa-miR-6738-5p”.

The term “hsa-miR-6741-5p gene” or “hsa-miR-6741-5p” used herein includes the hsa-miR-6741-5p gene (miRBase Accession No. MIMAT0027383) described in SEQ ID NO: 268, a homolog or an ortholog of a different organism species, and the like. The hsa-miR-6741-5p gene can be obtained by a method described in Ladewig E et al., 2012, Genome Res, Vol. 22, p. 1634-1645. Also, “hsa-mir-6741” (miRBase Accession No. MI0022586, SEQ ID NO: 607) having a hairpin-like structure is known as a precursor of “hsa-miR-6741-5p”.

The term “hsa-miR-6749-5p gene” or “hsa-miR-6749-5p” used herein includes the hsa-miR-6749-5p gene (miRBase Accession No. MIMAT0027398) described in SEQ ID NO: 269, a homolog or an ortholog of a different organism species, and the like. The hsa-miR-6749-5p gene can be obtained by a method described in Ladewig E et al., 2012, Genome Res, Vol. 22, p. 1634-1645. Also, “hsa-mir-6749” (miRBase Accession No. MI0022594, SEQ ID NO: 608) having a hairpin-like structure is known as a precursor of “hsa-miR-6749-5p”.

The term “hsa-miR-6752-5p gene” or “hsa-miR-6752-5p” used herein includes the hsa-miR-6752-5p gene (miRBase Accession No. MIMAT0027404) described in SEQ ID NO: 270, a homolog or an ortholog of a different organism species, and the like. The hsa-miR-6752-5p gene can be obtained by a method described in Ladewig E et al., 2012, Genome Res, Vol. 22, p. 1634-1645. Also, “hsa-mir-6752” (miRBase Accession No. MI0022597, SEQ ID NO: 609) having a hairpin-like structure is known as a precursor of “hsa-miR-6752-5p”.

The term “hsa-miR-675-5p gene” or “hsa-miR-675-5p” used herein includes the hsa-miR-675-5p gene (miRBase Accession No. MIMAT0004284) described in SEQ ID NO: 271, a homolog or an ortholog of a different organism species, and the like. The hsa-miR-675-5p gene can be obtained by a method described in Cai X et al., 2007, RNA, Vol. 13, p. 313-316. Also, “hsa-mir-675” (miRBase Accession No. MI0005416, SEQ ID NO: 610) having a hairpin-like structure is known as a precursor of “hsa-miR-675-5p”.

The term “hsa-miR-6757-5p gene” or “hsa-miR-6757-5p” used herein includes the hsa-miR-6757-5p gene (miRBase Accession No. MIMAT0027414) described in SEQ ID NO: 272, a homolog or an ortholog of a different organism species, and the like. The hsa-miR-6757-5p gene can be obtained by a method described in Ladewig E et al., 2012, Genome Res, Vol. 22, p. 1634-1645. Also, “hsa-mir-6757” (miRBase Accession No. MI0022602, SEQ ID NO: 611) having a hairpin-like structure is known as a precursor of “hsa-miR-6757-5p”.

The term “hsa-miR-6763-5p gene” or “hsa-miR-6763-5p” used herein includes the hsa-miR-6763-5p gene (miRBase Accession No. MIMAT0027426) described in SEQ ID NO: 273, a homolog or an ortholog of a different organism species, and the like. The hsa-miR-6763-5p gene can be obtained by a method described in Ladewig E et al., 2012, Genome Res, Vol. 22, p. 1634-1645. Also, “hsa-mir-6763” (miRBase Accession No. MI0022608, SEQ ID NO: 442) having a hairpin-like structure is known as a precursor of “hsa-miR-6763-5p”.

The term “hsa-miR-6765-5p gene” or “hsa-miR-6765-5p” used herein includes the hsa-miR-6765-5p gene (miRBase Accession No. MIMAT0027430) described in SEQ ID NO: 274, a homolog or an ortholog of a different organism species, and the like. The hsa-miR-6765-5p gene can be obtained by a method described in Ladewig E et al., 2012, Genome Res, Vol. 22, p. 1634-1645. Also, “hsa-mir-6765” (miRBase Accession No. MI0022610, SEQ ID NO: 612) having a hairpin-like structure is known as a precursor of “hsa-miR-6765-5p”.

The term “hsa-miR-6775-5p gene” or “hsa-miR-6775-5p” used herein includes the hsa-miR-6775-5p gene (miRBase Accession No. MIMAT0027450) described in SEQ ID NO: 275, a homolog or an ortholog of a different organism species, and the like. The hsa-miR-6775-5p gene can be obtained by a method described in Ladewig E et al., 2012, Genome Res, Vol. 22, p. 1634-1645. Also, “hsa-mir-6775” (miRBase Accession No. MI0022620, SEQ ID NO: 613) having a hairpin-like structure is known as a precursor of “hsa-miR-6775-5p”.

The term “hsa-miR-6780b-5p gene” or “hsa-miR-6780b-5p” used herein includes the hsa-miR-6780b-5p gene (miRBase Accession No. MIMAT0027572) described in SEQ ID NO: 276, a homolog or an ortholog of a different organism species, and the like. The hsa-miR-6780b-5p gene can be obtained by a method described in Ladewig E et al., 2012, Genome Res, Vol. 22, p. 1634-1645. Also, “hsa-mir-6780b” (miRBase Accession No. MI0022681, SEQ ID NO: 614) having a hairpin-like structure is known as a precursor of “hsa-miR-6780b-5p”.

The term “hsa-miR-6782-5p gene” or “hsa-miR-6782-5p” used herein includes the hsa-miR-6782-5p gene (miRBase Accession No. MIMAT0027464) described in SEQ ID NO: 277, a homolog or an ortholog of a different organism species, and the like. The hsa-miR-6782-5p gene can be obtained by a method described in Ladewig E et al., 2012, Genome Res, Vol. 22, p. 1634-1645. Also, “hsa-mir-6782” (miRBase Accession No. MI0022627, SEQ ID NO: 615) having a hairpin-like structure is known as a precursor of “hsa-miR-6782-5p”.

The term “hsa-miR-6784-5p gene” or “hsa-miR-6784-5p” used herein includes the hsa-miR-6784-5p gene (miRBase Accession No. MIMAT0027468) described in SEQ ID NO: 278, a homolog or an ortholog of a different organism species, and the like. The hsa-miR-6784-5p gene can be obtained by a method described in Ladewig E et al., 2012, Genome Res, Vol. 22, p. 1634-1645. Also, “hsa-mir-6784” (miRBase Accession No. MI0022629, SEQ ID NO: 616) having a hairpin-like structure is known as a precursor of “hsa-miR-6784-5p”.

The term “hsa-miR-6800-5p gene” or “hsa-miR-6800-5p” used herein includes the hsa-miR-6800-5p gene (miRBase Accession No. MIMAT0027500) described in SEQ ID NO: 279, a homolog or an ortholog of a different organism species, and the like. The hsa-miR-6800-5p gene can be obtained by a method described in Ladewig E et al., 2012, Genome Res, Vol. 22, p. 1634-1645. Also, “hsa-mir-6800” (miRBase Accession No. MI0022645, SEQ ID NO: 451) having a hairpin-like structure is known as a precursor of “hsa-miR-6800-5p”.

The term “hsa-miR-6806-5p gene” or “hsa-miR-6806-5p” used herein includes the hsa-miR-6806-5p gene (miRBase Accession No. MIMAT0027512) described in SEQ ID NO: 280, a homolog or an ortholog of a different organism species, and the like. The hsa-miR-6806-5p gene can be obtained by a method described in Ladewig E et al., 2012, Genome Res, Vol. 22, p. 1634-1645. Also, “hsa-mir-6806” (miRBase Accession No. MI0022651, SEQ ID NO: 617) having a hairpin-like structure is known as a precursor of “hsa-miR-6806-5p”.

The term “hsa-miR-6840-3p gene” or “hsa-miR-6840-3p” used herein includes the hsa-miR-6840-3p gene (miRBase Accession No. MIMAT0027583) described in SEQ ID NO: 281, a homolog or an ortholog of a different organism species, and the like. The hsa-miR-6840-3p gene can be obtained by a method described in Ladewig E et al., 2012, Genome Res, Vol. 22, p. 1634-1645. Also, “hsa-mir-6840” (miRBase Accession No. MI0022686, SEQ ID NO: 618) having a hairpin-like structure is known as a precursor of “hsa-miR-6840-3p”.

The term “hsa-miR-6848-5p gene” or “hsa-miR-6848-5p” used herein includes the hsa-miR-6848-5p gene (miRBase Accession No. MIMAT0027596) described in SEQ ID NO: 282, a homolog or an ortholog of a different organism species, and the like. The hsa-miR-6848-5p gene can be obtained by a method described in Ladewig E et al., 2012, Genome Res, Vol. 22, p. 1634-1645. Also, “hsa-mir-6848” (miRBase Accession No. MI0022694, SEQ ID NO: 619) having a hairpin-like structure is known as a precursor of “hsa-miR-6848-5p”.

The term “hsa-miR-6851-5p gene” or “hsa-miR-6851-5p” used herein includes the hsa-miR-6851-5p gene (miRBase Accession No. MIMAT0027602) described in SEQ ID NO: 283, a homolog or an ortholog of a different organism species, and the like. The hsa-miR-6851-5p gene can be obtained by a method described in Ladewig E et al., 2012, Genome Res, Vol. 22, p. 1634-1645. Also, “hsa-mir-6851” (miRBase Accession No. MI0022697, SEQ ID NO: 620) having a hairpin-like structure is known as a precursor of “hsa-miR-6851-5p”.

The term “hsa-miR-6870-5p gene” or “hsa-miR-6870-5p” used herein includes the hsa-miR-6870-5p gene (miRBase Accession No. MIMAT0027640) described in SEQ ID NO: 284, a homolog or an ortholog of a different organism species, and the like. The hsa-miR-6870-5p gene can be obtained by a method described in Ladewig E et al., 2012, Genome Res, Vol. 22, p. 1634-1645. Also, “hsa-mir-6870” (miRBase Accession No. MI0022717, SEQ ID NO: 621) having a hairpin-like structure is known as a precursor of “hsa-miR-6870-5p”.

The term “hsa-miR-6872-3p gene” or “hsa-miR-6872-3p” used herein includes the hsa-miR-6872-3p gene (miRBase Accession No. MIMAT0027645) described in SEQ ID NO: 285, a homolog or an ortholog of a different organism species, and the like. The hsa-miR-6872-3p gene can be obtained by a method described in Ladewig E et al., 2012, Genome Res, Vol. 22, p. 1634-1645. Also, “hsa-mir-6872” (miRBase Accession No. MI0022719, SEQ ID NO: 622) having a hairpin-like structure is known as a precursor of “hsa-miR-6872-3p”.

The term “hsa-miR-6875-5p gene” or “hsa-miR-6875-5p” used herein includes the hsa-miR-6875-5p gene (miRBase Accession No. MIMAT0027650) described in SEQ ID NO: 286, a homolog or an ortholog of a different organism species, and the like. The hsa-miR-6875-5p gene can be obtained by a method described in Ladewig E et al., 2012, Genome Res, Vol. 22, p. 1634-1645. Also, “hsa-mir-6875” (miRBase Accession No. MI0022722, SEQ ID NO: 623) having a hairpin-like structure is known as a precursor of “hsa-miR-6875-5p”.

The term “hsa-miR-6877-5p gene” or “hsa-miR-6877-5p” used herein includes the hsa-miR-6877-5p gene (miRBase Accession No. MIMAT0027654) described in SEQ ID NO: 287, a homolog or an ortholog of a different organism species, and the like. The hsa-miR-6877-5p gene can be obtained by a method described in Ladewig E et al., 2012, Genome Res, Vol. 22, p. 1634-1645. Also, “hsa-mir-6877” (miRBase Accession No. MI0022724, SEQ ID NO: 624) having a hairpin-like structure is known as a precursor of “hsa-miR-6877-5p”.

The term “hsa-miR-6879-5p gene” or “hsa-miR-6879-5p” used herein includes the hsa-miR-6879-5p gene (miRBase Accession No. MIMAT0027658) described in SEQ ID NO: 288, a homolog or an ortholog of a different organism species, and the like. The hsa-miR-6879-5p gene can be obtained by a method described in Ladewig E et al., 2012, Genome Res, Vol. 22, p. 1634-1645. Also, “hsa-mir-6879” (miRBase Accession No. MI0022726, SEQ ID NO: 625) having a hairpin-like structure is known as a precursor of “hsa-miR-6879-5p”.

The term “hsa-miR-6880-5p gene” or “hsa-miR-6880-5p” used herein includes the hsa-miR-6880-5p gene (miRBase Accession No. MIMAT0027660) described in SEQ ID NO: 289, a homolog or an ortholog of a different organism species, and the like. The hsa-miR-6880-5p gene can be obtained by a method described in Ladewig E et al., 2012, Genome Res, Vol. 22, p. 1634-1645. Also, “hsa-mir-6880” (miRBase Accession No. MI0022727, SEQ ID NO: 464) having a hairpin-like structure is known as a precursor of “hsa-miR-6880-5p”.

The term “hsa-miR-6885-5p gene” or “hsa-miR-6885-5p” used herein includes the hsa-miR-6885-5p gene (miRBase Accession No. MIMAT0027670) described in SEQ ID NO: 290, a homolog or an ortholog of a different organism species, and the like. The hsa-miR-6885-5p gene can be obtained by a method described in Ladewig E et al., 2012, Genome Res, Vol. 22, p. 1634-1645. Also, “hsa-mir-6885” (miRBase Accession No. MI0022732, SEQ ID NO: 626) having a hairpin-like structure is known as a precursor of “hsa-miR-6885-5p”.

The term “hsa-miR-6887-5p gene” or “hsa-miR-6887-5p” used herein includes the hsa-miR-6887-5p gene (miRBase Accession No. MIMAT0027674) described in SEQ ID NO: 291, a homolog or an ortholog of a different organism species, and the like. The hsa-miR-6887-5p gene can be obtained by a method described in Ladewig E et al., 2012, Genome Res, Vol. 22, p. 1634-1645. Also, “hsa-mir-6887” (miRBase Accession No. MI0022734, SEQ ID NO: 627) having a hairpin-like structure is known as a precursor of “hsa-miR-6887-5p”.

The term “hsa-miR-7108-5p gene” or “hsa-miR-7108-5p” used herein includes the hsa-miR-7108-5p gene (miRBase Accession No. MIMAT0028113) described in SEQ ID NO: 292, a homolog or an ortholog of a different organism species, and the like. The hsa-miR-7108-5p gene can be obtained by a method described in Ladewig E et al., 2012, Genome Res, Vol. 22, p. 1634-1645. Also, “hsa-mir-7108” (miRBase Accession No. MI0022959, SEQ ID NO: 495) having a hairpin-like structure is known as a precursor of “hsa-miR-7108-5p”.

The term “hsa-miR-711 gene” or “hsa-miR-711” used herein includes the hsa-miR-711 gene (miRBase Accession No. MIMAT0012734) described in SEQ ID NO: 293, a homolog or an ortholog of a different organism species, and the like. The hsa-miR-711 gene can be obtained by a method described in Artzi S et al., 2008, BMC Bioinformatics, Vol. 9, p. 39. Also, “hsa-mir-711” (miRBase Accession No. MI0012488, SEQ ID NO: 628) having a hairpin-like structure is known as a precursor of “hsa-miR-711”.

The term “hsa-miR-7113-3p gene” or “hsa-miR-7113-3p” used herein includes the hsa-miR-7113-3p gene (miRBase Accession No. MIMAT0028124) described in SEQ ID NO: 294, a homolog or an ortholog of a different organism species, and the like. The hsa-miR-7113-3p gene can be obtained by a method described in Ladewig E et al., 2012, Genome Res, Vol. 22, p. 1634-1645. Also, “hsa-mir-7113” (miRBase Accession No. MI0022964, SEQ ID NO: 629) having a hairpin-like structure is known as a precursor of “hsa-miR-7113-3p”.

The term “hsa-miR-744-5p gene” or “hsa-miR-744-5p” used herein includes the hsa-miR-744-5p gene (miRBase Accession No. MIMAT0004945) described in SEQ ID NO: 295, a homolog or an ortholog of a different organism species, and the like. The hsa-miR-744-5p gene can be obtained by a method described in Berezikov E et al., 2006, Genome Res, Vol. 16, p. 1289-1298. Also, “hsa-mir-744” (miRBase Accession No. MI0005559, SEQ ID NO: 630) having a hairpin-like structure is known as a precursor of “hsa-miR-744-5p”.

The term “hsa-miR-760 gene” or “hsa-miR-760” used herein includes the hsa-miR-760 gene (miRBase Accession No. MIMAT0004957) described in SEQ ID NO: 296, a homolog or an ortholog of a different organism species, and the like. The hsa-miR-760 gene can be obtained by a method described in Berezikov E et al., 2006, Genome Res, Vol. 16, p. 1289-1298. Also, “hsa-mir-760” (miRBase Accession No. MI0005567, SEQ ID NO: 631) having a hairpin-like structure is known as a precursor of “hsa-miR-760”.

The term “hsa-miR-7845-5p gene” or “hsa-miR-7845-5p” used herein includes the hsa-miR-7845-5p gene (miRBase Accession No. MIMAT0030420) described in SEQ ID NO: 297, a homolog or an ortholog of a different organism species, and the like. The hsa-miR-7845-5p gene can be obtained by a method described in Ple H et al., 2012, PLoS One, Vol. 7, e50746. Also, “hsa-mir-7845” (miRBase Accession No. MI0025515, SEQ ID NO: 632) having a hairpin-like structure is known as a precursor of “hsa-miR-7845-5p”.

The term “hsa-miR-7847-3p gene” or “hsa-miR-7847-3p” used herein includes the hsa-miR-7847-3p gene (miRBase Accession No. MIMAT0030422) described in SEQ ID NO: 298, a homolog or an ortholog of a different organism species, and the like. The hsa-miR-7847-3p gene can be obtained by a method described in Ple H et al., 2012, PLoS One, Vol. 7, e50746. Also, “hsa-mir-7847” (miRBase Accession No. MI0025517, SEQ ID NO: 633) having a hairpin-like structure is known as a precursor of “hsa-miR-7847-3p”.

The term “hsa-miR-7977 gene” or “hsa-miR-7977” used herein includes the hsa-miR-7977 gene (miRBase Accession No. MIMAT0031180) described in SEQ ID NO: 299, a homolog or an ortholog of a different organism species, and the like. The hsa-miR-7977 gene can be obtained by a method described in Velthut-Meikas A et al., 2013, Mol Endocrinol, online. Also, “hsa-mir-7977” (miRBase Accession No. MI0025753, SEQ ID NO: 634) having a hairpin-like structure is known as a precursor of “hsa-miR-7977”.

The term “hsa-miR-8059 gene” or “hsa-miR-8059” used herein includes the hsa-miR-8059 gene (miRBase Accession No. MIMAT0030986) described in SEQ ID NO: 300, a homolog or an ortholog of a different organism species, and the like. The hsa-miR-8059 gene can be obtained by a method described in Wang H J et al., 2013, Shock, Vol. 39, p. 480-487. Also, “hsa-mir-8059” (miRBase Accession No. MI0025895, SEQ ID NO: 635) having a hairpin-like structure is known as a precursor of “hsa-miR-8059”.

The term “hsa-miR-8063 gene” or “hsa-miR-8063” used herein includes the hsa-miR-8063 gene (miRBase Accession No. MIMAT0030990) described in SEQ ID NO: 301, a homolog or an ortholog of a different organism species, and the like. The hsa-miR-8063 gene can be obtained by a method described in Wang H J et al., 2013, Shock, Vol. 39, p. 480-487. Also, “hsa-mir-8063” (miRBase Accession No. MI0025899, SEQ ID NO: 636) having a hairpin-like structure is known as a precursor of “hsa-miR-8063”.

The term “hsa-miR-8072 gene” or “hsa-miR-8072” used herein includes the hsa-miR-8072 gene (miRBase Accession No. MIMAT0030999) described in SEQ ID NO: 302, a homolog or an ortholog of a different organism species, and the like. The hsa-miR-8072 gene can be obtained by a method described in Wang H J et al., 2013, Shock, Vol. 39, p. 480-487. Also, “hsa-mir-8072” (miRBase Accession No. MI0025908, SEQ ID NO: 637) having a hairpin-like structure is known as a precursor of “hsa-miR-8072”.

The term “hsa-miR-874-3p gene” or “hsa-miR-874-3p” used herein includes the hsa-miR-874-3p gene (miRBase Accession No. MIMAT0004911) described in SEQ ID NO: 303, a homolog or an ortholog of a different organism species, and the like. The hsa-miR-874-3p gene can be obtained by a method described in Landgraf P et al., 2007, Cell., Vol. 129, p. 1401-1414. Also, “hsa-mir-874” (miRBase Accession No. MI0005532, SEQ ID NO: 475) having a hairpin-like structure is known as a precursor of “hsa-miR-874-3p”.

The term “hsa-miR-92a-2-5p gene” or “hsa-miR-92a-2-5p” used herein includes the hsa-miR-92a-2-5p gene (miRBase Accession No. MIMAT0004508) described in SEQ ID NO: 304, a homolog or an ortholog of a different organism species, and the like. The hsa-miR-92a-2-5p gene can be obtained by a method described in Mourelatos Z et al., 2002, Genes Dev, Vol. 16, p. 720-728. Also, “hsa-mir-92a-2” (miRBase Accession No. MI0000094, SEQ ID NO: 638) having a hairpin-like structure is known as a precursor of “hsa-miR-92a-2-5p”.

The term “hsa-miR-92b-5p gene” or “hsa-miR-92b-5p” used herein includes the hsa-miR-92b-5p gene (miRBase Accession No. MIMAT0004792) described in SEQ ID NO: 305, a homolog or an ortholog of a different organism species, and the like. The hsa-miR-92b-5p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 499) having a hairpin-like structure is known as a precursor of “hsa-miR-92b-5p”.

The term “hsa-miR-940 gene” or “hsa-miR-940” used herein includes the hsa-miR-940 gene (miRBase Accession No. MIMAT0004983) described in SEQ ID NO: 306, a homolog or an ortholog of a different organism species, and the like. The hsa-miR-940 gene can be obtained by a method described in Lui W O et al., 2007, Cancer Res, Vol. 67, p. 6031-6043. Also, “hsa-mir-940” (miRBase Accession No. MI0005762, SEQ ID NO: 639) having a hairpin-like structure is known as a precursor of “hsa-miR-940”.

The term “hsa-miR-1228-3p gene” or “hsa-miR-1228-3p” used herein includes the hsa-miR-1228-3p gene (miRBase Accession No. MIMAT0005583) described in SEQ ID NO: 307, a homolog or an ortholog of a different organism species, and the like. The hsa-miR-1228-3p gene can be obtained by a method described in Berezikov E et al., 2007, Mol Cell, Vol. 28, p. 328-336. Also, “hsa-mir-1228” (miRBase Accession No. MI0006318, SEQ ID NO: 511) having a hairpin-like structure is known as a precursor of “hsa-miR-1228-3p”.

The term “hsa-miR-1275 gene” or “hsa-miR-1275” used herein includes the hsa-miR-1275 gene (miRBase Accession No. MIMAT0005929) described in SEQ ID NO: 308, a homolog or an ortholog of a different organism species, and the like. The hsa-miR-1275 gene can be obtained by a method described in Morin R D et al., 2008, Genome Res, Vol. 18, p. 610-621. Also, “hsa-mir-1275” (miRBase Accession No. MI0006415, SEQ ID NO: 640) having a hairpin-like structure is known as a precursor of “hsa-miR-1275”.

The term “hsa-miR-1307-3p gene” or “hsa-miR-1307-3p” used herein includes the hsa-miR-1307-3p gene (miRBase Accession No. MIMAT0005951) described in SEQ ID NO: 309, a homolog or an ortholog of a different organism species, and the like. The hsa-miR-1307-3p gene can be obtained by a method described in Morin R D et al., 2008, Genome Res, Vol. 18, p. 610-621. Also, “hsa-mir-1307” (miRBase Accession No. MI0006444, SEQ ID NO: 641) having a hairpin-like structure is known as a precursor of “hsa-miR-1307-3p”.

The term “hsa-miR-1343-5p gene” or “hsa-miR-1343-5p” used herein includes the hsa-miR-1343-5p gene (miRBase Accession No. MIMAT0027038) described in SEQ ID NO: 310, a homolog or an ortholog of a different organism species, and the like. The hsa-miR-1343-5p gene can be obtained by a method described in Persson H et al., 2011, Cancer Res, Vol. 71, p. 78-86. Also, “hsa-mir-1343” (miRBase Accession No. MI0017320, SEQ ID NO: 500) having a hairpin-like structure is known as a precursor of “hsa-miR-1343-5p”.

The term “hsa-miR-23a-3p gene” or “hsa-miR-23a-3p” used herein includes the hsa-miR-23a-3p gene (miRBase Accession No. MIMAT0000078) described in SEQ ID NO: 311, a homolog or an ortholog of a different organism species, and the like. The hsa-miR-23a-3p gene can be obtained by a method described in Lagos-Quintana M et al., 2001, Science, Vol. 294, p. 853-858. Also, “hsa-mir-23a” (miRBase Accession No. MI0000079, SEQ ID NO: 642) having a hairpin-like structure is known as a precursor of “hsa-miR-23a-3p”.

The term “hsa-miR-29b-3p gene” or “hsa-miR-29b-3p” used herein includes the hsa-miR-29b-3p gene (miRBase Accession No. MIMAT0000100) described in SEQ ID NO: 312, a homolog or an ortholog of a different organism species, and the like. The hsa-miR-29b-3p gene can be obtained by a method described in Mourelatos Z et al., 2002, Genes Dev., Vol. 16, p. 720-728. Also, “hsa-mir-29b-1 and hsa-mir-29b-2” (miRBase Accession Nos. MI0000105 and MI0000107, SEQ ID NOs: 643 and 644) having a hairpin-like structure are known as precursors of “hsa-miR-29b-3p”.

The term “hsa-miR-3135b gene” or “hsa-miR-3135b” used herein includes the hsa-miR-3135b gene (miRBase Accession No. MIMAT0018985) described in SEQ ID NO: 313, a homolog or an ortholog of a different organism species, and the like. The hsa-miR-3135b gene can be obtained by a method described in Jima D D et al., 2010, Blood, Vol. 116, e118-e127. Also, “hsa-mir-3135b” (miRBase Accession No. MI0016809, SEQ ID NO: 645) having a hairpin-like structure is known as a precursor of “hsa-miR-3135b”.

The term “hsa-miR-3185 gene” or “hsa-miR-3185” used herein includes the hsa-miR-3185 gene (miRBase Accession No. MIMAT0015065) described in SEQ ID NO: 314, a homolog or an ortholog of a different organism species, and the like. The hsa-miR-3185 gene can be obtained by a method described in Stark M S et al., 2010, PLoS One, Vol. 5, e9685. Also, “hsa-mir-3185” (miRBase Accession No. MI0014227, SEQ ID NO: 646) having a hairpin-like structure is known as a precursor of “hsa-miR-3185”.

The term “hsa-miR-4532 gene” or “hsa-miR-4532” used herein includes the hsa-miR-4532 gene (miRBase Accession No. MIMAT0019071) described in SEQ ID NO: 315, a homolog or an ortholog of a different organism species, and the like. The hsa-miR-4532 gene can be obtained by a method described in Jima D D et al., 2010, Blood, Vol. 116, e118-e127. Also, “hsa-mir-4532” (miRBase Accession No. MI0016899, SEQ ID NO: 647) having a hairpin-like structure is known as a precursor of “hsa-miR-4532”.

The term “hsa-miR-4690-5p gene” or “hsa-miR-4690-5p” used herein includes the hsa-miR-4690-5p gene (miRBase Accession No. MIMAT0019779) described in SEQ ID NO: 316, a homolog or an ortholog of a different organism species, and the like. The hsa-miR-4690-5p gene can be obtained by a method described in Persson H et al., 2011, Cancer Res, Vol. 71, p. 78-86. Also, “hsa-mir-4690” (miRBase Accession No. MI0017323, SEQ ID NO: 648) having a hairpin-like structure is known as a precursor of “hsa-miR-4690-5p”.

The term “hsa-miR-4758-5p gene” or “hsa-miR-4758-5p” used herein includes the hsa-miR-4758-5p gene (miRBase Accession No. MIMAT0019903) described in SEQ ID NO: 317, a homolog or an ortholog of a different organism species, and the like. The hsa-miR-4758-5p gene can be obtained by a method described in Persson H et al., 2011, Cancer Res, Vol. 71, p. 78-86. Also, “hsa-mir-4758” (miRBase Accession No. MI0017399, SEQ ID NO: 649) having a hairpin-like structure is known as a precursor of “hsa-miR-4758-5p”.

The term “hsa-miR-4783-3p gene” or “hsa-miR-4783-3p” used herein includes the hsa-miR-4783-3p gene (miRBase Accession No. MIMAT0019947) described in SEQ ID NO: 318, a homolog or an ortholog of a different organism species, and the like. The hsa-miR-4783-3p gene can be obtained by a method described in Persson H et al., 2011, Cancer Res, Vol. 71, p. 78-86. Also, “hsa-mir-4783” (miRBase Accession No. MI0017428, SEQ ID NO: 650) having a hairpin-like structure is known as a precursor of “hsa-miR-4783-3p”.

The term “hsa-miR-6131 gene” or “hsa-miR-6131” used herein includes the hsa-miR-6131 gene (miRBase Accession No. MIMAT0024615) described in SEQ ID NO: 319, a homolog or an ortholog of a different organism species, and the like. The hsa-miR-6131 gene can be obtained by a method described in Dannemann M et al., 2012, Genome Biol Evol, Vol. 4, p. 552-564. Also, “hsa-mir-6131” (miRBase Accession No. MI0021276, SEQ ID NO: 651) having a hairpin-like structure is known as a precursor of “hsa-miR-6131”.

The term “hsa-miR-625-3p gene” or “hsa-miR-625-3p” used herein includes the hsa-miR-625-3p gene (miRBase Accession No. MIMAT0004808) described in SEQ ID NO: 320, a homolog or an ortholog of a different organism species, and the like. The hsa-miR-625-3p gene can be obtained by a method described in Cummins J M et al., 2006, Proc Natl Acad Sci USA, Vol. 103, p. 3687-3692. Also, “hsa-mir-625” (miRBase Accession No. MI0003639, SEQ ID NO: 652) having a hairpin-like structure is known as a precursor of “hsa-miR-625-3p”.

The term “hsa-miR-6511a-5p gene” or “hsa-miR-6511a-5p” used herein includes the hsa-miR-6511a-5p gene (miRBase Accession No. MIMAT0025478) described in SEQ ID NO: 321, a homolog or an ortholog of a different organism species, and the like. The hsa-miR-6511a-5p gene can be obtained by a method described in Joyce C E et al., 2011, Hum Mol Genet, Vol. 20, p. 4025-4040. Also, “hsa-mir-6511a-1, hsa-mir-6511a-2, hsa-mir-6511a-3, and hsa-mir-6511a-4” (miRBase Accession Nos. MI0022223, MI0023564, MI0023565, and MI0023566, SEQ ID NOs: 653, 654, 655, and 656) having a hairpin-like structure are known as precursors of “hsa-miR-6511a-5p”.

The term “hsa-miR-6765-3p gene” or “hsa-miR-6765-3p” used herein includes the hsa-miR-6765-3p gene (miRBase Accession No. MIMAT0027431) described in SEQ ID NO: 322, a homolog or an ortholog of a different organism species, and the like. The hsa-miR-6765-3p gene can be obtained by a method described in Ladewig E et al., 2012, Genome Res, Vol. 22, p. 1634-1645. Also, “hsa-mir-6765” (miRBase Accession No. MI0022610, SEQ ID NO: 612) having a hairpin-like structure is known as a precursor of “hsa-miR-6765-3p”.

The term “hsa-miR-6816-5p gene” or “hsa-miR-6816-5p” used herein includes the hsa-miR-6816-5p gene (miRBase Accession No. MIMAT0027532) described in SEQ ID NO: 323, a homolog or an ortholog of a different organism species, and the like. The hsa-miR-6816-5p gene can be obtained by a method described in Ladewig E et al., 2012, Genome Res, Vol. 22, p. 1634-1645. Also, “hsa-mir-6816” (miRBase Accession No. MI0022661, SEQ ID NO: 657) having a hairpin-like structure is known as a precursor of “hsa-miR-6816-5p”.

The term “hsa-miR-6825-5p gene” or “hsa-miR-6825-5p” used herein includes the hsa-miR-6825-5p gene (miRBase Accession No. MIMAT0027550) described in SEQ ID NO: 324, a homolog or an ortholog of a different organism species, and the like. The hsa-miR-6825-5p gene can be obtained by a method described in Ladewig E et al., 2012, Genome Res, Vol. 22, p. 1634-1645. Also, “hsa-mir-6825” (miRBase Accession No. MI0022670, SEQ ID NO: 658) having a hairpin-like structure is known as a precursor of “hsa-miR-6825-5p”.

The term “hsa-miR-6845-5p gene” or “hsa-miR-6845-5p” used herein includes the hsa-miR-6845-5p gene (miRBase Accession No. MIMAT0027590) described in SEQ ID NO: 325, a homolog or an ortholog of a different organism species, and the like. The hsa-miR-6845-5p gene can be obtained by a method described in Ladewig E et al., 2012, Genome Res, Vol. 22, p. 1634-1645. Also, “hsa-mir-6845” (miRBase Accession No. MI0022691, SEQ ID NO: 659) having a hairpin-like structure is known as a precursor of “hsa-miR-6845-5p”.

The term “hsa-miR-7150 gene” or “hsa-miR-7150” used herein includes the hsa-miR-7150 gene (miRBase Accession No. MIMAT0028211) described in SEQ ID NO: 326, a homolog or an ortholog of a different organism species, and the like. The hsa-miR-7150 gene can be obtained by a method described in Oulas A et al., 2009, Nucleic Acids Res, Vol. 37, p. 3276-3287. Also, “hsa-mir-7150” (miRBase Accession No. MI0023610, SEQ ID NO: 660) having a hairpin-like structure is known as a precursor of “hsa-miR-7150”.

The term “hsa-miR-7641 gene” or “hsa-miR-7641” used herein includes the hsa-miR-7641 gene (miRBase Accession No. MIMAT0029782) described in SEQ ID NO: 327, a homolog or an ortholog of a different organism species, and the like. The hsa-miR-7641 gene can be obtained by a method described in Yoo J K et al., 2013, Arch Pharm Res, Vol. 36, p. 353-358. Also, “hsa-mir-7641-1 and hsa-mir-7641-2” (miRBase Accession Nos. MI0024975 and MI0024976, SEQ ID NOs: 661 and 662) having a hairpin-like structure are known as precursors of “hsa-miR-7641”.

The term “hsa-miR-7975 gene” or “hsa-miR-7975” used herein includes the hsa-miR-7975 gene (miRBase Accession No. MIMAT0031178) described in SEQ ID NO: 328, a homolog or an ortholog of a different organism species, and the like. The hsa-miR-7975 gene can be obtained by a method described in Velthut-Meikas A et al., 2013, Mol Endocrinol, online. Also, “hsa-mir-7975” (miRBase Accession No. MI0025751, SEQ ID NO: 663) having a hairpin-like structure is known as a precursor of “hsa-miR-7975”.

The term “hsa-miR-92a-3p gene” or “hsa-miR-92a-3p” used herein includes the hsa-miR-92a-3p gene (miRBase Accession No. MIMAT0000092) described in SEQ ID NO: 329, a homolog or an ortholog of a different organism species, and the like. The hsa-miR-92a-3p gene can be obtained by a method described in Mourelatos Z et al., 2002, Genes Dev, Vol. 16, p. 720-728. Also, “hsa-mir-92a-1 and hsa-mir-92a-2” (miRBase Accession Nos. MI0000093 and MI0000094, SEQ ID NOs: 664 and 638) having a hairpin-like structure are known as precursors of “hsa-miR-92a-3p”.

A mature miRNA may become a variant shorter or longer by one to several flanking nucleotides due to the sequence cleavage, or due to substitution of nucleotides, when cut out as the mature miRNA from its RNA precursor having a hairpin-like structure. This variant is called isomiR (Morin R D. et al., 2008, Genome Res., Vol. 18, p. 610-621). The miRBase Release 21 shows the nucleotide sequences represented by SEQ ID NOs: 1 to 329 as well as a large number of the nucleotide sequence variants and fragments represented by SEQ ID NOs: 665 to 1000, called isomiRs. These variants can also be obtained as miRNAs having a nucleotide sequence represented by any of SEQ ID NOs: 1 to 329. Specifically, according to the present invention, among the variants of polynucleotides consisting of the nucleotide sequence represented by any of SEQ ID NOs: 4, 7, 8, 9, 13, 14, 18, 20, 21, 22, 23, 26, 28, 31, 32, 33, 35, 36, 38, 41, 44, 45, 46, 48, 49, 51, 52, 53, 54, 55, 56, 57, 58, 61, 62, 68, 73, 74, 77, 78, 82, 83, 84, 85, 86, 87, 91, 92, 93, 94, 95, 96, 97, 100, 101, 138, 139, 141, 145, 146, 147, 150, 151, 163, 164, 167, 170, 171, 175, 177, 179, 180, 181, 182, 183, 184, 185, 188, 189, 190, 191, 192, 193, 195, 196, 197, 198, 200, 201, 202, 203, 204, 206, 207, 209, 210, 211, 212, 214, 216, 220, 223, 224, 226, 227, 229, 230, 231, 233, 235, 237, 240, 241, 244, 245, 246, 249, 252, 253, 254, 256, 257, 258, 259, 260, 261, 262, 263, 264, 295, 296, 303, 304, 305, 306, 307, 308, 309, 311, 312, 313, 315, 316, 317, 318, 319, 320, 321, and 329, or the nucleotide sequence in which the nucleic acid u is replaced with t, examples of the longest variants registered in miRBase Release 21 include polynucleotides represented by SEQ ID NOs: 666, 668, 669, 671, 674, 676, 679, 681, 683, 685, 687, 691, 693, 697, 699, 701, 703, 705, 707, 709, 712, 713, 715, 717, 719, 721, 723, 724, 726, 728, 730, 732, 734, 736, 738, 743, 748, 750, 752, 754, 757, 759, 761, 763, 765, 767, 770, 772, 774, 776, 778, 779, 781, 783, 785, 787, 789, 792, 795, 797, 799, 803, 805, 808, 810, 812, 815, 817, 819, 821, 824, 826, 828, 830, 832, 834, 836, 840, 842, 844, 846, 848, 850, 853, 855, 857, 859, 862, 864, 866, 868, 870, 873, 875, 877, 879, 881, 883, 886, 889, 892, 896, 898, 901, 903, 904, 906, 908, 911, 912, 914, 917, 919, 923, 925, 927, 930, 933, 935, 937, 940, 942, 944, 946, 948, 950, 952, 954, 956, 960, 962, 964, 966, 968, 970, 972, 974, 976, 978, 980, 982, 985, 987, 989, 991, 993, 994, 996, and 999, respectively. Also, according to the present invention, among the variants of polynucleotides consisting of the nucleotide sequence represented by any of SEQ ID NOs: 3, 4, 8, 9, 10, 13, 14, 17, 18, 20, 21, 22, 23, 24, 25, 26, 28, 29, 30, 31, 32, 33, 35, 36, 38, 41, 43, 45, 46, 48, 49, 51, 53, 54, 55, 56, 57, 58, 61, 62, 65, 66, 67, 68, 69, 71, 72, 73, 74, 77, 78, 80, 82, 83, 84, 85, 86, 87, 89, 91, 92, 93, 94, 96, 97, 100, 114, 138, 139, 140, 141, 142, 145, 146, 147, 148, 149, 150, 151, 162, 163, 164, 167, 168, 170, 171, 175, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 209, 210, 211, 212, 213, 214, 215, 216, 219, 220, 221, 222, 223, 224, 225, 226, 229, 230, 231, 232, 235, 237, 238, 240, 241, 242, 243, 244, 245, 246, 248, 249, 251, 252, 253, 254, 255, 256, 257, 258, 259, 260, 261, 262, 263, 264, 271, 293, 295, 296, 303, 304, 305, 306, 307, 308, 309, 311, 312, 313, 314, 315, 316, 317, 318, 320, 321, 328, and 329, or the nucleotide sequence in which the nucleic acid u is replaced with t, examples of the shortest variants registered in the miRBase Release 21 include polynucleotides having sequences represented by SEQ ID NOs: 665, 667, 670, 672, 673, 675, 677, 678, 680, 682, 684, 686, 688, 689, 690, 692, 694, 695, 696, 698, 700, 702, 704, 706, 708, 710, 711, 714, 716, 718, 720, 722, 725, 727, 729, 731, 733, 735, 737, 739, 740, 741, 742, 744, 745, 746, 747, 749, 751, 753, 755, 756, 758, 760, 762, 764, 766, 768, 769, 771, 773, 775, 777, 780, 782, 784, 786, 788, 790, 791, 793, 794, 796, 798, 800, 801, 802, 804, 806, 807, 809, 811, 813, 814, 816, 818, 820, 822, 823, 825, 827, 829, 831, 833, 835, 837, 838, 839, 841, 843, 845, 847, 849, 851, 852, 854, 856, 858, 860, 861, 863, 865, 867, 869, 871, 872, 874, 876, 878, 880, 882, 884, 885, 887, 888, 890, 891, 893, 894, 895, 897, 899, 900, 902, 905, 907, 909, 910, 913, 915, 916, 918, 920, 921, 922, 924, 926, 928, 929, 931, 932, 934, 936, 938, 939, 941, 943, 945, 947, 949, 951, 953, 955, 957, 958, 959, 961, 963, 965, 967, 969, 971, 973, 975, 977, 979, 981, 983, 984, 986, 988, 990, 992, 995, 997, 998, and 1000, respectively. In addition to these variants and fragments, examples thereof include a large number of isomiR polynucleotides of SEQ ID NOs: 1 to 329 registered in the miRBase. Examples of the polynucleotide comprising a nucleotide sequence represented by any of SEQ ID NOs: 1 to 329 include a polynucleotide represented by any of SEQ ID NOs: 330 to 664, which are their respective precursors.

The names and miRBase Accession Nos. (registration numbers) of the genes represented by SEQ ID NOs: 1 to 1000 are shown in Table 1.

As used herein, the term “capable of specifically binding” means that nucleic acids such as the nucleic acid probe or the primer used in the present invention binds to a particular target nucleic acid and cannot substantially bind to other nucleic acids.

TABLE 1

| SEQ ID | | Accession No. |
|---|---|---|
| NO: | Name of gene | of miRBase |
| 1 | hsa-miR-6787-5p | MIMAT0027474 |
| 2 | hsa-miR-920 | MIMAT0004970 |
| 3 | hsa-miR-3622a-5p | MIMAT0018003 |
| 4 | hsa-miR-1185-1-3p | MIMAT0022838 |
| 5 | hsa-miR-4327 | MIMAT0016889 |
| 6 | hsa-miR-5739 | MIMAT0023116 |
| 7 | hsa-miR-937-5p | MIMAT0022938 |
| 8 | hsa-miR-1181 | MIMAT0005826 |
| 9 | hsa-miR-1185-2-3p | MIMAT0022713 |
| 10 | hsa-miR-1193 | MIMAT0015049 |
| 11 | hsa-miR-1207-5p | MIMAT0005871 |
| 12 | hsa-miR-1238-5p | MIMAT0022947 |
| 13 | hsa-miR-1246 | MIMAT0005898 |
| 14 | hsa-miR-1249-5p | MIMAT0032029 |
| 15 | hsa-miR-1292-3p | MIMAT0022948 |
| 16 | hsa-miR-1469 | MIMAT0007347 |
| 17 | hsa-miR-1470 | MIMAT0007348 |
| 18 | hsa-miR-197-5p | MIMAT0022691 |
| 19 | hsa-miR-208a-5p | MIMAT0026474 |
| 20 | hsa-miR-2110 | MIMAT0010133 |
| 21 | hsa-miR-211-3p | MIMAT0022694 |
| 22 | hsa-miR-2467-3p | MIMAT0019953 |
| 23 | hsa-miR-3122 | MIMAT0014984 |
| 24 | hsa-miR-3141 | MIMAT0015010 |
| 25 | hsa-miR-3156-5p | MIMAT0015030 |
| 26 | hsa-miR-3158-5p | MIMAT0019211 |
| 27 | hsa-miR-3160-5p | MIMAT0019212 |
| 28 | hsa-miR-3180-3p | MIMAT0015058 |
| 29 | hsa-miR-3191-3p | MIMAT0015075 |
| 30 | hsa-miR-3194-3p | MIMAT0019218 |
| 31 | hsa-miR-320b | MIMAT0005792 |
| 32 | hsa-miR-328-5p | MIMAT0026486 |
| 33 | hsa-miR-3610 | MIMAT0017987 |
| 34 | hsa-miR-3619-3p | MIMAT0019219 |
| 35 | hsa-miR-3620-5p | MIMAT0022967 |
| 36 | hsa-miR-370-3p | MIMAT0000722 |
| 37 | hsa-miR-373-5p | MIMAT0000725 |
| 38 | hsa-miR-3917 | MIMAT0018191 |
| 39 | hsa-miR-3937 | MIMAT0018352 |
| 40 | hsa-miR-4259 | MIMAT0016880 |
| 41 | hsa-miR-4281 | MIMAT0016907 |
| 42 | hsa-miR-4294 | MIMAT0016849 |
| 43 | hsa-miR-4419b | MIMAT0019034 |
| 44 | hsa-miR-4428 | MIMAT0018943 |
| 45 | hsa-miR-4429 | MIMAT0018944 |
| 46 | hsa-miR-4433a-3p | MIMAT0018949 |
| 47 | hsa-miR-4447 | MIMAT0018966 |
| 48 | hsa-miR-4449 | MIMAT0018968 |
| 49 | hsa-miR-4459 | MIMAT0018981 |
| 50 | hsa-miR-4480 | MIMAT0019014 |
| 51 | hsa-miR-4485-5p | MIMAT0032116 |
| 52 | hsa-miR-4486 | MIMAT0019020 |
| 53 | hsa-miR-4488 | MIMAT0019022 |
| 54 | hsa-miR-4489 | MIMAT0019023 |
| 55 | hsa-miR-4505 | MIMAT0019041 |
| 56 | hsa-miR-4513 | MIMAT0019050 |
| 57 | hsa-miR-4515 | MIMAT0019052 |
| 58 | hsa-miR-4530 | MIMAT0019069 |
| 59 | hsa-miR-4535 | MIMAT0019075 |
| 60 | hsa-miR-4635 | MIMAT0019692 |
| 61 | hsa-miR-4640-5p | MIMAT0019699 |
| 62 | hsa-miR-4646-5p | MIMAT0019707 |
| 63 | hsa-miR-4656 | MIMAT0019723 |
| 64 | hsa-miR-4663 | MIMAT0019735 |
| 65 | hsa-miR-4665-5p | MIMAT0019739 |
| 66 | hsa-miR-4706 | MIMAT0019806 |
| 67 | hsa-miR-4707-5p | MIMAT0019807 |
| 68 | hsa-miR-4708-3p | MIMAT0019810 |
| 69 | hsa-miR-4710 | MIMAT0019815 |
| 70 | hsa-miR-4718 | MIMAT0019831 |
| 71 | hsa-miR-4722-5p | MIMAT0019836 |
| 72 | hsa-miR-4727-3p | MIMAT0019848 |
| 73 | hsa-miR-4730 | MIMAT0019852 |
| 74 | hsa-miR-4734 | MIMAT0019859 |
| 75 | hsa-miR-4740-5p | MIMAT0019869 |
| 76 | hsa-miR-4747-3p | MIMAT0019883 |
| 77 | hsa-miR-4749-5p | MIMAT0019885 |
| 78 | hsa-miR-4755-3p | MIMAT0019896 |
| 79 | hsa-miR-4763-5p | MIMAT0019912 |
| 80 | hsa-miR-4787-3p | MIMAT0019957 |
| 81 | hsa-miR-5008-5p | MIMAT0021039 |
| 82 | hsa-miR-5010-5p | MIMAT0021043 |
| 83 | hsa-miR-504-3p | MIMAT0026612 |
| 84 | hsa-miR-5090 | MIMAT0021082 |
| 85 | hsa-miR-5100 | MIMAT0022259 |
| 86 | hsa-miR-5196-5p | MIMAT0021128 |
| 87 | hsa-miR-551b-5p | MIMAT0004794 |
| 88 | hsa-miR-557 | MIMAT0003221 |
| 89 | hsa-miR-5787 | MIMAT0023252 |
| 90 | hsa-miR-6090 | MIMAT0023715 |
| 91 | hsa-miR-6124 | MIMAT0024597 |
| 92 | hsa-miR-6132 | MIMAT0024616 |
| 93 | hsa-miR-6510-5p | MIMAT0025476 |
| 94 | hsa-miR-6511b-5p | MIMAT0025847 |
| 95 | hsa-miR-6515-3p | MIMAT0025487 |
| 96 | hsa-miR-654-5p | MIMAT0003330 |
| 97 | hsa-miR-658 | MIMAT0003336 |
| 98 | hsa-miR-668-5p | MIMAT0026636 |
| 99 | hsa-miR-6722-5p | MIMAT0025853 |
| 100 | hsa-miR-6724-5p | MIMAT0025856 |
| 101 | hsa-miR-6729-3p | MIMAT0027360 |
| 102 | hsa-miR-6737-5p | MIMAT0027375 |
| 103 | hsa-miR-6756-5p | MIMAT0027412 |
| 104 | hsa-miR-6762-5p | MIMAT0027424 |
| 105 | hsa-miR-6763-3p | MIMAT0027427 |
| 106 | hsa-miR-6766-5p | MIMAT0027432 |
| 107 | hsa-miR-6769a-5p | MIMAT0027438 |
| 108 | hsa-miR-6771-5p | MIMAT0027442 |
| 109 | hsa-miR-6786-5p | MIMAT0027472 |
| 110 | hsa-miR-6789-5p | MIMAT0027478 |
| 111 | hsa-miR-6794-5p | MIMAT0027488 |
| 112 | hsa-miR-6796-3p | MIMAT0027493 |
| 113 | hsa-miR-6797-5p | MIMAT0027494 |
| 114 | hsa-miR-6800-3p | MIMAT0027501 |
| 115 | hsa-miR-6802-5p | MIMAT0027504 |
| 116 | hsa-miR-6803-5p | MIMAT0027506 |
| 117 | hsa-miR-6805-3p | MIMAT0027511 |
| 118 | hsa-miR-6805-5p | MIMAT0027510 |
| 119 | hsa-miR-6807-5p | MIMAT0027514 |
| 120 | hsa-miR-6812-5p | MIMAT0027524 |
| 121 | hsa-miR-6819-5p | MIMAT0027538 |
| 122 | hsa-miR-6822-5p | MIMAT0027544 |
| 123 | hsa-miR-6824-5p | MIMAT0027548 |
| 124 | hsa-miR-6826-5p | MIMAT0027552 |
| 125 | hsa-miR-6850-5p | MIMAT0027600 |
| 126 | hsa-miR-6858-5p | MIMAT0027616 |
| 127 | hsa-miR-6861-5p | MIMAT0027623 |
| 128 | hsa-miR-6880-3p | MIMAT0027661 |
| 129 | hsa-miR-7107-5p | MIMAT0028111 |
| 130 | hsa-miR-7109-5p | MIMAT0028115 |
| 131 | hsa-miR-7114-5p | MIMAT0028125 |
| 132 | hsa-miR-7704 | MIMAT0030019 |
| 133 | hsa-miR-7846-3p | MIMAT0030421 |
| 134 | hsa-miR-8052 | MIMAT0030979 |
| 135 | hsa-miR-8060 | MIMAT0030987 |
| 136 | hsa-miR-8071 | MIMAT0030998 |
| 137 | hsa-miR-8073 | MIMAT0031000 |
| 138 | hsa-miR-874-5p | MIMAT0026718 |
| 139 | hsa-miR-204-3p | MIMAT0022693 |
| 140 | hsa-miR-3154 | MIMAT0015028 |
| 141 | hsa-miR-3960 | MIMAT0019337 |
| 142 | hsa-miR-4433a-5p | MIMAT0020956 |
| 143 | hsa-miR-4455 | MIMAT0018977 |
| 144 | hsa-miR-4462 | MIMAT0018986 |
| 145 | hsa-miR-4476 | MIMAT0019003 |
| 146 | hsa-miR-4508 | MIMAT0019045 |
| 147 | hsa-miR-4687-3p | MIMAT0019775 |
| 148 | hsa-miR-4687-5p | MIMAT0019774 |
| 149 | hsa-miR-4732-5p | MIMAT0019855 |
| 150 | hsa-miR-4771 | MIMAT0019925 |
| 151 | hsa-miR-642a-3p | MIMAT0020924 |
| 152 | hsa-miR-6732-5p | MIMAT0027365 |
| 153 | hsa-miR-6760-5p | MIMAT0027420 |
| 154 | hsa-miR-6799-5p | MIMAT0027498 |
| 155 | hsa-miR-6820-5p | MIMAT0027540 |
| 156 | hsa-miR-6821-5p | MIMAT0027542 |
| 157 | hsa-miR-6829-5p | MIMAT0027558 |
| 158 | hsa-miR-6893-5p | MIMAT0027686 |
| 159 | hsa-miR-7108-3p | MIMAT0028114 |
| 160 | hsa-miR-7111-5p | MIMAT0028119 |
| 161 | hsa-miR-8089 | MIMAT0031016 |
| 162 | hsa-miR-885-3p | MIMAT0004948 |
| 163 | hsa-miR-92b-3p | MIMAT0003218 |
| 164 | hsa-miR-1343-3p | MIMAT0019776 |
| 165 | hsa-miR-6746-5p | MIMAT0027392 |
| 166 | hsa-miR-422a | MIMAT0001339 |
| 167 | hsa-miR-187-5p | MIMAT0004561 |
| 168 | hsa-miR-4632-5p | MIMAT0022977 |
| 169 | hsa-miR-6791-5p | MIMAT0027482 |
| 170 | hsa-miR-103a-3p | MIMAT0000101 |
| 171 | hsa-miR-107 | MIMAT0000104 |
| 172 | hsa-miR-1199-5p | MIMAT0031119 |
| 173 | hsa-miR-1225-3p | MIMAT0005573 |
| 174 | hsa-miR-1225-5p | MIMAT0005572 |
| 175 | hsa-miR-1228-5p | MIMAT0005582 |
| 176 | hsa-miR-1229-5p | MIMAT0022942 |
| 177 | hsa-miR-1233-5p | MIMAT0022943 |
| 178 | hsa-miR-1237-5p | MIMAT0022946 |
| 179 | hsa-miR-1247-3p | MIMAT0022721 |
| 180 | hsa-miR-1249-3p | MIMAT0005901 |
| 181 | hsa-miR-1254 | MIMAT0005905 |
| 182 | hsa-miR-1260b | MIMAT0015041 |
| 183 | hsa-miR-1268a | MIMAT0005922 |
| 184 | hsa-miR-1268b | MIMAT0018925 |
| 185 | hsa-miR-1273g-3p | MIMAT0022742 |
| 186 | hsa-miR-128-1-5p | MIMAT0026477 |
| 187 | hsa-miR-128-2-5p | MIMAT0031095 |
| 188 | hsa-miR-1290 | MIMAT0005880 |
| 189 | hsa-miR-150-3p | MIMAT0004610 |
| 190 | hsa-miR-17-3p | MIMAT0000071 |
| 191 | hsa-miR-1908-5p | MIMAT0007881 |
| 192 | hsa-miR-1909-3p | MIMAT0007883 |
| 193 | hsa-miR-1914-3p | MIMAT0007890 |
| 194 | hsa-miR-1915-3p | MIMAT0007892 |
| 195 | hsa-miR-191-5p | MIMAT0000440 |
| 196 | hsa-miR-22-3p | MIMAT0000077 |
| 197 | hsa-miR-23b-3p | MIMAT0000418 |
| 198 | hsa-miR-24-3p | MIMAT0000080 |
| 199 | hsa-miR-296-3p | MIMAT0004679 |
| 200 | hsa-miR-296-5p | MIMAT0000690 |
| 201 | hsa-miR-3131 | MIMAT0014996 |
| 202 | hsa-miR-3162-5p | MIMAT0015036 |
| 203 | hsa-miR-3188 | MIMAT0015070 |
| 204 | hsa-miR-3196 | MIMAT0015080 |
| 205 | hsa-miR-3197 | MIMAT0015082 |
| 206 | hsa-miR-320a | MIMAT0000510 |
| 207 | hsa-miR-342-5p | MIMAT0004694 |
| 208 | hsa-miR-3621 | MIMAT0018002 |
| 209 | hsa-miR-3648 | MIMAT0018068 |
| 210 | hsa-miR-3656 | MIMAT0018076 |
| 211 | hsa-miR-365a-5p | MIMAT0009199 |
| 212 | hsa-miR-3665 | MIMAT0018087 |
| 213 | hsa-miR-3679-5p | MIMAT0018104 |
| 214 | hsa-miR-371a-5p | MIMAT0004687 |
| 215 | hsa-miR-3940-5p | MIMAT0019229 |
| 216 | hsa-miR-423-5p | MIMAT0004748 |
| 217 | hsa-miR-4257 | MIMAT0016878 |
| 218 | hsa-miR-4270 | MIMAT0016900 |
| 219 | hsa-miR-4271 | MIMAT0016901 |
| 220 | hsa-miR-4286 | MIMAT0016916 |
| 221 | hsa-miR-4298 | MIMAT0016852 |
| 222 | hsa-miR-4417 | MIMAT0018929 |
| 223 | hsa-miR-4442 | MIMAT0018960 |
| 224 | hsa-miR-4446-3p | MIMAT0018965 |
| 225 | hsa-miR-4448 | MIMAT0018967 |
| 226 | hsa-miR-4454 | MIMAT0018976 |
| 227 | hsa-miR-4467 | MIMAT0018994 |
| 228 | hsa-miR-4472 | MIMAT0018999 |
| 229 | hsa-miR-4507 | MIMAT0019044 |
| 230 | hsa-miR-4516 | MIMAT0019053 |
| 231 | hsa-miR-451a | MIMAT0001631 |
| 232 | hsa-miR-4649-5p | MIMAT0019711 |
| 233 | hsa-miR-4651 | MIMAT0019715 |
| 234 | hsa-miR-4665-3p | MIMAT0019740 |
| 235 | hsa-miR-4674 | MIMAT0019756 |
| 236 | hsa-miR-4675 | MIMAT0019757 |
| 237 | hsa-miR-4689 | MIMAT0019778 |
| 238 | hsa-miR-4695-5p | MIMAT0019788 |
| 239 | hsa-miR-4697-5p | MIMAT0019791 |
| 240 | hsa-miR-4725-3p | MIMAT0019844 |
| 241 | hsa-miR-4739 | MIMAT0019868 |
| 242 | hsa-miR-4745-5p | MIMAT0019878 |
| 243 | hsa-miR-4763-3p | MIMAT0019913 |
| 244 | hsa-miR-4792 | MIMAT0019964 |
| 245 | hsa-miR-486-3p | MIMAT0004762 |
| 246 | hsa-miR-5001-5p | MIMAT0021021 |
| 247 | hsa-miR-5195-3p | MIMAT0021127 |
| 248 | hsa-miR-550a-5p | MIMAT0004800 |
| 249 | hsa-miR-5698 | MIMAT0022491 |
| 250 | hsa-miR-6075 | MIMAT0023700 |
| 251 | hsa-miR-6088 | MIMAT0023713 |
| 252 | hsa-miR-6089 | MIMAT0023714 |
| 253 | hsa-miR-6125 | MIMAT0024598 |
| 254 | hsa-miR-6126 | MIMAT0024599 |
| 255 | hsa-miR-614 | MIMAT0003282 |
| 256 | hsa-miR-615-5p | MIMAT0004804 |
| 257 | hsa-miR-619-5p | MIMAT0026622 |
| 258 | hsa-miR-638 | MIMAT0003308 |
| 259 | hsa-miR-642b-3p | MIMAT0018444 |
| 260 | hsa-miR-650 | MIMAT0003320 |
| 261 | hsa-miR-663a | MIMAT0003326 |
| 262 | hsa-miR-663b | MIMAT0005867 |
| 263 | hsa-miR-6717-5p | MIMAT0025846 |
| 264 | hsa-miR-6721-5p | MIMAT0025852 |
| 265 | hsa-miR-6726-5p | MIMAT0027353 |
| 266 | hsa-miR-6727-5p | MIMAT0027355 |
| 267 | hsa-miR-6738-5p | MIMAT0027377 |
| 268 | hsa-miR-6741-5p | MIMAT0027383 |
| 269 | hsa-miR-6749-5p | MIMAT0027398 |
| 270 | hsa-miR-6752-5p | MIMAT0027404 |
| 271 | hsa-miR-675-5p | MIMAT0004284 |
| 272 | hsa-miR-6757-5p | MIMAT0027414 |
| 273 | hsa-miR-6763-5p | MIMAT0027426 |
| 274 | hsa-miR-6765-5p | MIMAT0027430 |
| 275 | hsa-miR-6775-5p | MIMAT0027450 |
| 276 | hsa-miR-6780b-5p | MIMAT0027572 |
| 277 | hsa-miR-6782-5p | MIMAT0027464 |
| 278 | hsa-miR-6784-5p | MIMAT0027468 |
| 279 | hsa-miR-6800-5p | MIMAT0027500 |
| 280 | hsa-miR-6806-5p | MIMAT0027512 |
| 281 | hsa-miR-6840-3p | MIMAT0027583 |
| 282 | hsa-miR-6848-5p | MIMAT0027596 |
| 283 | hsa-miR-6851-5p | MIMAT0027602 |
| 284 | hsa-miR-6870-5p | MIMAT0027640 |
| 285 | hsa-miR-6872-3p | MIMAT0027645 |
| 286 | hsa-miR-6875-5p | MIMAT0027650 |
| 287 | hsa-miR-6877-5p | MIMAT0027654 |
| 288 | hsa-miR-6879-5p | MIMAT0027658 |
| 289 | hsa-miR-6880-5p | MIMAT0027660 |
| 290 | hsa-miR-6885-5p | MIMAT0027670 |
| 291 | hsa-miR-6887-5p | MIMAT0027674 |
| 292 | hsa-miR-7108-5p | MIMAT0028113 |
| 293 | hsa-miR-711 | MIMAT0012734 |
| 294 | hsa-miR-7113-3p | MIMAT0028124 |
| 295 | hsa-miR-744-5p | MIMAT0004945 |
| 296 | hsa-miR-760 | MIMAT0004957 |
| 297 | hsa-miR-7845-5p | MIMAT0030420 |
| 298 | hsa-miR-7847-3p | MIMAT0030422 |
| 299 | hsa-miR-7977 | MIMAT0031180 |
| 300 | hsa-miR-8059 | MIMAT0030986 |
| 301 | hsa-miR-8063 | MIMAT0030990 |
| 302 | hsa-miR-8072 | MIMAT0030999 |
| 303 | hsa-miR-874-3p | MIMAT0004911 |
| 304 | hsa-miR-92a-2-5p | MIMAT0004508 |
| 305 | hsa-miR-92b-5p | MIMAT0004792 |
| 306 | hsa-miR-940 | MIMAT0004983 |
| 307 | hsa-miR-1228-3p | MIMAT0005583 |
| 308 | hsa-miR-1275 | MIMAT0005929 |
| 309 | hsa-miR-1307-3p | MIMAT0005951 |
| 310 | hsa-miR-1343-5p | MIMAT0027038 |
| 311 | hsa-miR-23a-3p | MIMAT0000078 |
| 312 | hsa-miR-29b-3p | MIMAT0000100 |
| 313 | hsa-miR-3135b | MIMAT0018985 |
| 314 | hsa-miR-3185 | MIMAT0015065 |
| 315 | hsa-miR-4532 | MIMAT0019071 |
| 316 | hsa-miR-4690-5p | MIMAT0019779 |
| 317 | hsa-miR-4758-5p | MIMAT0019903 |
| 318 | hsa-miR-4783-3p | MIMAT0019947 |
| 319 | hsa-miR-6131 | MIMAT0024615 |
| 320 | hsa-miR-625-3p | MIMAT0004808 |
| 321 | hsa-miR-6511a-5p | MIMAT0025478 |
| 322 | hsa-miR-6765-3p | MIMAT0027431 |
| 323 | hsa-miR-6816-5p | MIMAT0027532 |
| 324 | hsa-miR-6825-5p | MIMAT0027550 |
| 325 | hsa-miR-6845-5p | MIMAT0027590 |
| 326 | hsa-miR-7150 | MIMAT0028211 |
| 327 | hsa-miR-7641 | MIMAT0029782 |
| 328 | hsa-miR-7975 | MIMAT0031178 |
| 329 | hsa-miR-92a-3p | MIMAT0000092 |
| 330 | hsa-mir-6787 | MI0022632 |
| 331 | hsa-mir-920 | MI0005712 |
| 332 | hsa-mir-3622a | MI0016013 |
| 333 | hsa-mir-1185-1 | MI0003844 |
| 334 | hsa-mir-4327 | MI0015867 |
| 335 | hsa-mir-5739 | MI0019412 |
| 336 | hsa-mir-937 | MI0005759 |
| 337 | hsa-mir-1181 | MI0006274 |
| 338 | hsa-mir-1185-2 | MI0003821 |
| 339 | hsa-mir-1193 | MI0014205 |
| 340 | hsa-mir-1207 | MI0006340 |
| 341 | hsa-mir-1238 | MI0006328 |
| 342 | hsa-mir-1246 | MI0006381 |
| 343 | hsa-mir-1249 | MI0006384 |
| 344 | hsa-mir-1292 | MI0006433 |
| 345 | hsa-mir-1469 | MI0007074 |
| 346 | hsa-mir-1470 | MI0007075 |
| 347 | hsa-mir-197 | MI0000239 |
| 348 | hsa-mir-208a | MI0000251 |
| 349 | hsa-mir-2110 | MI0010629 |
| 350 | hsa-mir-211 | MI0000287 |
| 351 | hsa-mir-2467 | MI0017432 |
| 352 | hsa-mir-3122 | MI0014138 |
| 353 | hsa-mir-3141 | MI0014165 |
| 354 | hsa-mir-3156-1 | MI0014184 |
| 355 | hsa-mir-3156-2 | MI0014230 |
| 356 | hsa-mir-3156-3 | MI0014242 |
| 357 | hsa-mir-3158-1 | MI0014186 |
| 358 | hsa-mir-3158-2 | MI0014187 |
| 359 | hsa-mir-3160-1 | MI0014189 |
| 360 | hsa-mir-3160-2 | MI0014190 |
| 361 | hsa-mir-3180-1 | MI0014214 |
| 362 | hsa-mir-3180-2 | MI0014215 |
| 363 | hsa-mir-3180-3 | MI0014217 |
| 364 | hsa-mir-3191 | MI0014236 |
| 365 | hsa-mir-3194 | MI0014239 |
| 366 | hsa-mir-320b-1 | MI0003776 |
| 367 | hsa-mir-320b-2 | MI0003839 |
| 368 | hsa-mir-328 | MI0000804 |
| 369 | hsa-mir-3610 | MI0016000 |
| 370 | hsa-mir-3619 | MI0016009 |
| 371 | hsa-mir-3620 | MI0016011 |
| 372 | hsa-mir-370 | MI0000778 |
| 373 | hsa-mir-373 | MI0000781 |
| 374 | hsa-mir-3917 | MI0016423 |
| 375 | hsa-mir-3937 | MI0016593 |
| 376 | hsa-mir-4259 | MI0015858 |
| 377 | hsa-mir-4281 | MI0015885 |
| 378 | hsa-mir-4294 | MI0015827 |
| 379 | hsa-mir-4419b | MI0016861 |
| 380 | hsa-mir-4428 | MI0016767 |
| 381 | hsa-mir-4429 | MI0016768 |
| 382 | hsa-mir-4433a | MI0016773 |
| 383 | hsa-mir-4447 | MI0016790 |
| 384 | hsa-mir-4449 | MI0016792 |
| 385 | hsa-mir-4459 | MI0016805 |
| 386 | hsa-mir-4480 | MI0016841 |
| 387 | hsa-mir-4485 | MI0016846 |
| 388 | hsa-mir-4486 | MI0016847 |
| 389 | hsa-mir-4488 | MI0016849 |
| 390 | hsa-mir-4489 | MI0016850 |
| 391 | hsa-mir-4505 | MI0016868 |
| 392 | hsa-mir-4513 | MI0016879 |
| 393 | hsa-mir-4515 | MI0016881 |
| 394 | hsa-mir-4530 | MI0016897 |
| 395 | hsa-mir-4535 | MI0016903 |
| 396 | hsa-mir-4635 | MI0017262 |
| 397 | hsa-mir-4640 | MI0017267 |
| 398 | hsa-mir-4646 | MI0017273 |
| 399 | hsa-mir-4656 | MI0017284 |
| 400 | hsa-mir-4663 | MI0017292 |
| 401 | hsa-mir-4665 | MI0017295 |
| 402 | hsa-mir-4706 | MI0017339 |
| 403 | hsa-mir-4707 | MI0017340 |
| 404 | hsa-mir-4708 | MI0017341 |
| 405 | hsa-mir-4710 | MI0017344 |
| 406 | hsa-mir-4718 | MI0017353 |
| 407 | hsa-mir-4722 | MI0017357 |
| 408 | hsa-mir-4727 | MI0017364 |
| 409 | hsa-mir-4730 | MI0017367 |
| 410 | hsa-mir-4734 | MI0017371 |
| 411 | hsa-mir-4740 | MI0017378 |
| 412 | hsa-mir-4747 | MI0017386 |
| 413 | hsa-mir-4749 | MI0017388 |
| 414 | hsa-mir-4755 | MI0017395 |
| 415 | hsa-mir-4763 | MI0017404 |
| 416 | hsa-mir-4787 | MI0017434 |
| 417 | hsa-mir-5008 | MI0017876 |
| 418 | hsa-mir-5010 | MI0017878 |
| 419 | hsa-mir-504 | MI0003189 |
| 420 | hsa-mir-5090 | MI0017979 |
| 421 | hsa-mir-5100 | MI0019116 |
| 422 | hsa-mir-5196 | MI0018175 |
| 423 | hsa-mir-551b | MI0003575 |
| 424 | hsa-mir-557 | MI0003563 |
| 425 | hsa-mir-5787 | MI0019797 |
| 426 | hsa-mir-6090 | MI0020367 |
| 427 | hsa-mir-6124 | MI0021258 |
| 428 | hsa-mir-6132 | MI0021277 |
| 429 | hsa-mir-6510 | MI0022222 |
| 430 | hsa-mir-6511b-1 | MI0022552 |
| 431 | hsa-mir-6511b-2 | MI0023431 |
| 432 | hsa-mir-6515 | MI0022227 |
| 433 | hsa-mir-654 | MI0003676 |
| 434 | hsa-mir-658 | MI0003682 |
| 435 | hsa-mir-668 | MI0003761 |
| 436 | hsa-mir-6722 | MI0022557 |
| 437 | hsa-mir-6724 | MI0022559 |
| 438 | hsa-mir-6729 | MI0022574 |
| 439 | hsa-mir-6737 | MI0022582 |
| 440 | hsa-mir-6756 | MI0022601 |
| 441 | hsa-mir-6762 | MI0022607 |
| 442 | hsa-mir-6763 | MI0022608 |
| 443 | hsa-mir-6766 | MI0022611 |
| 444 | hsa-mir-6769a | MI0022614 |
| 445 | hsa-mir-6771 | MI0022616 |
| 446 | hsa-mir-6786 | MI0022631 |
| 447 | hsa-mir-6789 | MI0022634 |
| 448 | hsa-mir-6794 | MI0022639 |
| 449 | hsa-mir-6796 | MI0022641 |
| 450 | hsa-mir-6797 | MI0022642 |
| 451 | hsa-mir-6800 | MI0022645 |
| 452 | hsa-mir-6802 | MI0022647 |
| 453 | hsa-mir-6803 | MI0022648 |
| 454 | hsa-mir-6805 | MI0022650 |
| 455 | hsa-mir-6807 | MI0022652 |
| 456 | hsa-mir-6812 | MI0022657 |
| 457 | hsa-mir-6819 | MI0022664 |
| 458 | hsa-mir-6822 | MI0022667 |
| 459 | hsa-mir-6824 | MI0022669 |
| 460 | hsa-mir-6826 | MI0022671 |
| 461 | hsa-mir-6850 | MI0022696 |
| 462 | hsa-mir-6858 | MI0022704 |
| 463 | hsa-mir-6861 | MI0022708 |
| 464 | hsa-mir-6880 | MI0022727 |
| 465 | hsa-mir-7107 | MI0022958 |
| 466 | hsa-mir-7109 | MI0022960 |
| 467 | hsa-mir-7114 | MI0022965 |
| 468 | hsa-mir-7704 | MI0025240 |
| 469 | hsa-mir-7846 | MI0025516 |
| 470 | hsa-mir-8052 | MI0025888 |
| 471 | hsa-mir-8060 | MI0025896 |
| 472 | hsa-mir-8071-1 | MI0025907 |
| 473 | hsa-mir-8071-2 | MI0026417 |
| 474 | hsa-mir-8073 | MI0025909 |
| 475 | hsa-mir-874 | MI0005532 |
| 476 | hsa-mir-204 | MI0000284 |
| 477 | hsa-mir-3154 | MI0014182 |
| 478 | hsa-mir-3960 | MI0016964 |
| 479 | hsa-mir-4455 | MI0016801 |
| 480 | hsa-mir-4462 | MI0016810 |
| 481 | hsa-mir-4476 | MI0016828 |
| 482 | hsa-mir-4508 | MI0016872 |
| 483 | hsa-mir-4687 | MI0017319 |
| 484 | hsa-mir-4732 | MI0017369 |
| 485 | hsa-mir-4771-1 | MI0017412 |
| 486 | hsa-mir-4771-2 | MI0017413 |
| 487 | hsa-mir-642a | MI0003657 |
| 488 | hsa-mir-6732 | MI0022577 |
| 489 | hsa-mir-6760 | MI0022605 |
| 490 | hsa-mir-6799 | MI0022644 |
| 491 | hsa-mir-6820 | MI0022665 |
| 492 | hsa-mir-6821 | MI0022666 |
| 493 | hsa-mir-6829 | MI0022674 |
| 494 | hsa-mir-6893 | MI0022740 |
| 495 | hsa-mir-7108 | MI0022959 |
| 496 | hsa-mir-7111 | MI0022962 |
| 497 | hsa-mir-8089 | MI0025925 |
| 498 | hsa-mir-885 | MI0005560 |
| 499 | hsa-mir-92b | MI0003560 |
| 500 | hsa-mir-1343 | MI0017320 |
| 501 | hsa-mir-6746 | MI0022591 |
| 502 | hsa-mir-422a | MI0001444 |
| 503 | hsa-mir-187 | MI0000274 |
| 504 | hsa-mir-4632 | MI0017259 |
| 505 | hsa-mir-6791 | MI0022636 |
| 506 | hsa-mir-103a-2 | MI0000109 |
| 507 | hsa-mir-103a-1 | MI0000108 |
| 508 | hsa-mir-107 | MI0000114 |
| 509 | hsa-mir-1199 | MI0020340 |
| 510 | hsa-mir-1225 | MI0006311 |
| 511 | hsa-mir-1228 | MI0006318 |
| 512 | hsa-mir-1229 | MI0006319 |
| 513 | hsa-mir-1233-1 | MI0006323 |
| 514 | hsa-mir-1233-2 | MI0015973 |
| 515 | hsa-mir-1237 | MI0006327 |
| 516 | hsa-mir-1247 | MI0006382 |
| 517 | hsa-mir-1254-1 | MI0006388 |
| 518 | hsa-mir-1254-2 | MI0016747 |
| 519 | hsa-mir-1260b | MI0014197 |
| 520 | hsa-mir-1268a | MI0006405 |
| 521 | hsa-mir-1268b | MI0016748 |
| 522 | hsa-mir-1273g | MI0018003 |
| 523 | hsa-mir-128-1 | MI0000447 |
| 524 | hsa-mir-128-2 | MI0000727 |
| 525 | hsa-mir-1290 | MI0006352 |
| 526 | hsa-mir-150 | MI0000479 |
| 527 | hsa-mir-17 | MI0000071 |
| 528 | hsa-mir-1908 | MI0008329 |
| 529 | hsa-mir-1909 | MI0008330 |
| 530 | hsa-mir-1914 | MI0008335 |
| 531 | hsa-mir-1915 | MI0008336 |
| 532 | hsa-mir-191 | MI0000465 |
| 533 | hsa-mir-22 | MI0000078 |
| 534 | hsa-mir-23b | MI0000439 |
| 535 | hsa-mir-24-1 | MI0000080 |
| 536 | hsa-mir-24-2 | MI0000081 |
| 537 | hsa-mir-296 | MI0000747 |
| 538 | hsa-mir-3131 | MI0014151 |
| 539 | hsa-mir-3162 | MI0014192 |
| 540 | hsa-mir-3188 | MI0014232 |
| 541 | hsa-mir-3196 | MI0014241 |
| 542 | hsa-mir-3197 | MI0014245 |
| 543 | hsa-mir-320a | MI0000542 |
| 544 | hsa-mir-342 | MI0000805 |
| 545 | hsa-mir-3621 | MI0016012 |
| 546 | hsa-mir-3648 | MI0016048 |
| 547 | hsa-mir-3656 | MI0016056 |
| 548 | hsa-mir-365a | MI0000767 |
| 549 | hsa-mir-3665 | MI0016066 |
| 550 | hsa-mir-3679 | MI0016080 |
| 551 | hsa-mir-371a | MI0000779 |
| 552 | hsa-mir-3940 | MI0016597 |
| 553 | hsa-mir-423 | MI0001445 |
| 554 | hsa-mir-4257 | MI0015856 |
| 555 | hsa-mir-4270 | MI0015878 |
| 556 | hsa-mir-4271 | MI0015879 |
| 557 | hsa-mir-4286 | MI0015894 |
| 558 | hsa-mir-4298 | MI0015830 |
| 559 | hsa-mir-4417 | MI0016753 |
| 560 | hsa-mir-4442 | MI0016785 |
| 561 | hsa-mir-4446 | MI0016789 |
| 562 | hsa-mir-4448 | MI0016791 |
| 563 | hsa-mir-4454 | MI0016800 |
| 564 | hsa-mir-4467 | MI0016818 |
| 565 | hsa-mir-4472-1 | MI0016823 |
| 566 | hsa-mir-4472-2 | MI0016824 |
| 567 | hsa-mir-4507 | MI0016871 |
| 568 | hsa-mir-4516 | MI0016882 |
| 569 | hsa-mir-451a | MI0001729 |
| 570 | hsa-mir-4649 | MI0017276 |
| 571 | hsa-mir-4651 | MI0017279 |
| 572 | hsa-mir-4674 | MI0017305 |
| 573 | hsa-mir-4675 | MI0017306 |
| 574 | hsa-mir-4689 | MI0017322 |
| 575 | hsa-mir-4695 | MI0017328 |
| 576 | hsa-mir-4697 | MI0017330 |
| 577 | hsa-mir-4725 | MI0017362 |
| 578 | hsa-mir-4739 | MI0017377 |
| 579 | hsa-mir-4745 | MI0017384 |
| 580 | hsa-mir-4792 | MI0017439 |
| 581 | hsa-mir-486 | MI0002470 |
| 582 | hsa-mir-486-2 | MI0023622 |
| 583 | hsa-mir-5001 | MI0017867 |
| 584 | hsa-mir-5195 | MI0018174 |
| 585 | hsa-mir-550a-1 | MI0003600 |
| 586 | hsa-mir-550a-2 | MI0003601 |
| 587 | hsa-mir-5698 | MI0019305 |
| 588 | hsa-mir-6075 | MI0020352 |
| 589 | hsa-mir-6088 | MI0020365 |
| 590 | hsa-mir-6089-1 | MI0020366 |
| 591 | hsa-mir-6089-2 | MI0023563 |
| 592 | hsa-mir-6125 | MI0021259 |
| 593 | hsa-mir-6126 | MI0021260 |
| 594 | hsa-mir-614 | MI0003627 |
| 595 | hsa-mir-615 | MI0003628 |
| 596 | hsa-mir-619 | MI0003633 |
| 597 | hsa-mir-638 | MI0003653 |
| 598 | hsa-mir-642b | MI0016685 |
| 599 | hsa-mir-650 | MI0003665 |
| 600 | hsa-mir-663a | MI0003672 |
| 601 | hsa-mir-663b | MI0006336 |
| 602 | hsa-mir-6717 | MI0022551 |
| 603 | hsa-mir-6721 | MI0022556 |
| 604 | hsa-mir-6726 | MI0022571 |
| 605 | hsa-mir-6727 | MI0022572 |
| 606 | hsa-mir-6738 | MI0022583 |
| 607 | hsa-mir-6741 | MI0022586 |
| 608 | hsa-mir-6749 | MI0022594 |
| 609 | hsa-mir-6752 | MI0022597 |
| 610 | hsa-mir-675 | MI0005416 |
| 611 | hsa-mir-6757 | MI0022602 |
| 612 | hsa-mir-6765 | MI0022610 |
| 613 | hsa-mir-6775 | MI0022620 |
| 614 | hsa-mir-6780b | MI0022681 |
| 615 | hsa-mir-6782 | MI0022627 |
| 616 | hsa-mir-6784 | MI0022629 |
| 617 | hsa-mir-6806 | MI0022651 |
| 618 | hsa-mir-6840 | MI0022686 |
| 619 | hsa-mir-6848 | MI0022694 |
| 620 | hsa-mir-6851 | MI0022697 |
| 621 | hsa-mir-6870 | MI0022717 |
| 622 | hsa-mir-6872 | MI0022719 |
| 623 | hsa-mir-6875 | MI0022722 |
| 624 | hsa-mir-6877 | MI0022724 |
| 625 | hsa-mir-6879 | MI0022726 |
| 626 | hsa-mir-6885 | MI0022732 |
| 627 | hsa-mir-6887 | MI0022734 |
| 628 | hsa-mir-711 | MI0012488 |
| 629 | hsa-mir-7113 | MI0022964 |
| 630 | hsa-mir-744 | MI0005559 |
| 631 | hsa-mir-760 | MI0005567 |
| 632 | hsa-mir-7845 | MI0025515 |
| 633 | hsa-mir-7847 | MI0025517 |
| 634 | hsa-mir-7977 | MI0025753 |
| 635 | hsa-mir-8059 | MI0025895 |
| 636 | hsa-mir-8063 | MI0025899 |
| 637 | hsa-mir-8072 | MI0025908 |
| 638 | hsa-mir-92a-2 | MI0000094 |
| 639 | hsa-mir-940 | MI0005762 |
| 640 | hsa-mir-1275 | MI0006415 |
| 641 | hsa-mir-1307 | MI0006444 |
| 642 | hsa-mir-23a | MI0000079 |
| 643 | hsa-mir-29b-1 | MI0000105 |
| 644 | hsa-mir-29b-2 | MI0000107 |
| 645 | hsa-mir-3135b | MI0016809 |
| 646 | hsa-mir-3185 | MI0014227 |
| 647 | hsa-mir-4532 | MI0016899 |
| 648 | hsa-mir-4690 | MI0017323 |
| 649 | hsa-mir-4758 | MI0017399 |
| 650 | hsa-mir-4783 | MI0017428 |
| 651 | hsa-mir-6131 | MI0021276 |
| 652 | hsa-mir-625 | MI0003639 |
| 653 | hsa-mir-6511a-1 | MI0022223 |
| 654 | hsa-mir-6511a-2 | MI0023564 |
| 655 | hsa-mir-6511a-3 | MI0023565 |
| 656 | hsa-mir-6511a-4 | MI0023566 |
| 657 | hsa-mir-6816 | MI0022661 |
| 658 | hsa-mir-6825 | MI0022670 |
| 659 | hsa-mir-6845 | MI0022691 |
| 660 | hsa-mir-7150 | MI0023610 |
| 661 | hsa-mir-7641-1 | MI0024975 |
| 662 | hsa-mir-7641-2 | MI0024976 |
| 663 | hsa-mir-7975 | MI0025751 |
| 664 | hsa-mir-92a-1 | MI0000093 |
| 665 | isomiR Example 1 of SEQ ID NO: 3 | — |
| 666 | isomiR Example 1 of SEQ ID NO: 4 | — |
| 667 | isomiR Example 2 of SEQ ID NO: 4 | — |
| 668 | isomiR Example 1 of SEQ ID NO: 7 | — |
| 669 | isomiR Example 1 of SEQ ID NO: 8 | — |
| 670 | isomiR Example 2 of SEQ ID NO: 8 | — |
| 671 | isomiR Example 1 of SEQ ID NO: 9 | — |
| 672 | isomiR Example 2 of SEQ ID NO: 9 | — |
| 673 | isomiR Example 1 of SEQ ID NO: 10 | — |
| 674 | isomiR Example 1 of SEQ ID NO: 13 | — |
| 675 | isomiR Example 2 of SEQ ID NO: 13 | — |
| 676 | isomiR Example 1 of SEQ ID NO: 14 | — |
| 677 | isomiR Example 2 of SEQ ID NO: 14 | — |
| 678 | isomiR Example 1 of SEQ ID NO: 17 | — |
| 679 | isomiR Example 1 of SEQ ID NO: 18 | — |
| 680 | isomiR Example 2 of SEQ ID NO: 18 | — |
| 681 | isomiR Example 1 of SEQ ID NO: 20 | — |
| 682 | isomiR Example 2 of SEQ ID NO: 20 | — |
| 683 | isomiR Example 1 of SEQ ID NO: 21 | — |
| 684 | isomiR Example 2 of SEQ ID NO: 21 | — |
| 685 | isomiR Example 1 of SEQ ID NO: 22 | — |
| 686 | isomiR Example 2 of SEQ ID NO: 22 | — |
| 687 | isomiR Example 1 of SEQ ID NO: 23 | — |
| 688 | isomiR Example 2 of SEQ ID NO: 23 | — |
| 689 | isomiR Example 1 of SEQ ID NO: 24 | — |
| 690 | isomiR Example 1 of SEQ ID NO: 25 | — |
| 691 | isomiR Example 1 of SEQ ID NO: 26 | — |
| 692 | isomiR Example 2 of SEQ ID NO: 26 | — |
| 693 | isomiR Example 1 of SEQ ID NO: 28 | — |
| 694 | isomiR Example 2 of SEQ ID NO: 28 | — |
| 695 | isomiR Example 1 of SEQ ID NO: 29 | — |
| 696 | isomiR Example 1 of SEQ ID NO: 30 | — |
| 697 | isomiR Example 1 of SEQ ID NO: 31 | — |
| 698 | isomiR Example 2 of SEQ ID NO: 31 | — |
| 699 | isomiR Example 1 of SEQ ID NO: 32 | — |
| 700 | isomiR Example 2 of SEQ ID NO: 32 | — |
| 701 | isomiR Example 1 of SEQ ID NO: 33 | — |
| 702 | isomiR Example 2 of SEQ ID NO: 33 | — |
| 703 | isomiR Example 1 of SEQ ID NO: 35 | — |
| 704 | isomiR Example 2 of SEQ ID NO: 35 | — |
| 705 | isomiR Example 1 of SEQ ID NO: 36 | — |
| 706 | isomiR Example 2 of SEQ ID NO: 36 | — |
| 707 | isomiR Example 1 of SEQ ID NO: 38 | — |
| 708 | isomiR Example 2 of SEQ ID NO: 38 | — |
| 709 | isomiR Example 1 of SEQ ID NO: 41 | — |
| 710 | isomiR Example 2 of SEQ ID NO: 41 | — |
| 711 | isomiR Example 1 of SEQ ID NO: 43 | — |
| 712 | isomiR Example 1 of SEQ ID NO: 44 | — |
| 713 | isomiR Example 1 of SEQ ID NO: 45 | — |
| 714 | isomiR Example 2 of SEQ ID NO: 45 | — |
| 715 | isomiR Example 1 of SEQ ID NO: 46 | — |
| 716 | isomiR Example 2 of SEQ ID NO: 46 | — |
| 717 | isomiR Example 1 of SEQ ID NO: 48 | — |
| 718 | isomiR Example 2 of SEQ ID NO: 48 | — |
| 719 | isomiR Example 1 of SEQ ID NO: 49 | — |
| 720 | isomiR Example 2 of SEQ ID NO: 49 | — |
| 721 | isomiR Example 1 of SEQ ID NO: 51 | — |
| 722 | isomiR Example 2 of SEQ ID NO: 51 | — |
| 723 | isomiR Example 1 of SEQ ID NO: 52 | — |
| 724 | isomiR Example 1 of SEQ ID NO: 53 | — |
| 725 | isomiR Example 2 of SEQ ID NO: 53 | — |
| 726 | isomiR Example 1 of SEQ ID NO: 54 | — |
| 727 | isomiR Example 2 of SEQ ID NO: 54 | — |
| 728 | isomiR Example 1 of SEQ ID NO: 55 | — |
| 729 | isomiR Example 2 of SEQ ID NO: 55 | — |
| 730 | isomiR Example 1 of SEQ ID NO: 56 | — |
| 731 | isomiR Example 2 of SEQ ID NO: 56 | — |
| 732 | isomiR Example 1 of SEQ ID NO: 57 | — |
| 733 | isomiR Example 2 of SEQ ID NO: 57 | — |
| 734 | isomiR Example 1 of SEQ ID NO: 58 | — |
| 735 | isomiR Example 2 of SEQ ID NO: 58 | — |
| 736 | isomiR Example 1 of SEQ ID NO: 61 | — |
| 737 | isomiR Example 2 of SEQ ID NO: 61 | — |
| 738 | isomiR Example 1 of SEQ ID NO: 62 | — |
| 739 | isomiR Example 2 of SEQ ID NO: 62 | — |
| 740 | isomiR Example 1 of SEQ ID NO: 65 | — |
| 741 | isomiR Example 1 of SEQ ID NO: 66 | — |
| 742 | isomiR Example 1 of SEQ ID NO: 67 | — |
| 743 | isomiR Example 1 of SEQ ID NO: 68 | — |
| 744 | isomiR Example 2 of SEQ ID NO: 68 | — |
| 745 | isomiR Example 1 of SEQ ID NO: 69 | — |
| 746 | isomiR Example 1 of SEQ ID NO: 71 | — |
| 747 | isomiR Example 1 of SEQ ID NO: 72 | — |
| 748 | isomiR Example 1 of SEQ ID NO: 73 | — |
| 749 | isomiR Example 2 of SEQ ID NO: 73 | — |
| 750 | isomiR Example 1 of SEQ ID NO: 74 | — |
| 751 | isomiR Example 2 of SEQ ID NO: 74 | — |
| 752 | isomiR Example 1 of SEQ ID NO: 77 | — |
| 753 | isomiR Example 2 of SEQ ID NO: 77 | — |
| 754 | isomiR Example 1 of SEQ ID NO: 78 | — |
| 755 | isomiR Example 2 of SEQ ID NO: 78 | — |
| 756 | isomiR Example 1 of SEQ ID NO: 80 | — |
| 757 | isomiR Example 1 of SEQ ID NO: 82 | — |
| 758 | isomiR Example 2 of SEQ ID NO: 82 | — |
| 759 | isomiR Example 1 of SEQ ID NO: 83 | — |
| 760 | isomiR Example 2 of SEQ ID NO: 83 | — |
| 761 | isomiR Example 1 of SEQ ID NO: 84 | — |
| 762 | isomiR Example 2 of SEQ ID NO: 84 | — |
| 763 | isomiR Example 1 of SEQ ID NO: 85 | — |
| 764 | isomiR Example 2 of SEQ ID NO: 85 | — |
| 765 | isomiR Example 1 of SEQ ID NO: 86 | — |
| 766 | isomiR Example 2 of SEQ ID NO: 86 | — |
| 767 | isomiR Example 1 of SEQ ID NO: 87 | — |
| 768 | isomiR Example 2 of SEQ ID NO: 87 | — |
| 769 | isomiR Example 1 of SEQ ID NO: 89 | — |
| 770 | isomiR Example 1 of SEQ ID NO: 91 | — |
| 771 | isomiR Example 2 of SEQ ID NO: 91 | — |
| 772 | isomiR Example 1 of SEQ ID NO: 92 | — |
| 773 | isomiR Example 2 of SEQ ID NO: 92 | — |
| 774 | isomiR Example 1 of SEQ ID NO: 93 | — |
| 775 | isomiR Example 2 of SEQ ID NO: 93 | — |
| 776 | isomiR Example 1 of SEQ ID NO: 94 | — |
| 777 | isomiR Example 2 of SEQ ID NO: 94 | — |
| 778 | isomiR Example 1 of SEQ ID NO: 95 | — |
| 779 | isomiR Example 1 of SEQ ID NO: 96 | — |
| 780 | isomiR Example 2 of SEQ ID NO: 96 | — |
| 781 | isomiR Example 1 of SEQ ID NO: 97 | — |
| 782 | isomiR Example 2 of SEQ ID NO: 97 | — |
| 783 | isomiR Example 1 of SEQ ID NO: 100 | — |
| 784 | isomiR Example 2 of SEQ ID NO: 100 | — |
| 785 | isomiR Example 1 of SEQ ID NO: 101 | — |
| 786 | isomiR Example 1 of SEQ ID NO: 114 | — |
| 787 | isomiR Example 1 of SEQ ID NO: 138 | — |
| 788 | isomiR Example 2 of SEQ ID NO: 138 | — |
| 789 | isomiR Example 1 of SEQ ID NO: 139 | — |
| 790 | isomiR Example 2 of SEQ ID NO: 139 | — |
| 791 | isomiR Example 1 of SEQ ID NO: 140 | — |
| 792 | isomiR Example 1 of SEQ ID NO: 141 | — |
| 793 | isomiR Example 2 of SEQ ID NO: 141 | — |
| 794 | isomiR Example 1 of SEQ ID NO: 142 | — |
| 795 | isomiR Example 1 of SEQ ID NO: 145 | — |
| 796 | isomiR Example 2 of SEQ ID NO: 145 | — |
| 797 | isomiR Example 1 of SEQ ID NO: 146 | — |
| 798 | isomiR Example 2 of SEQ ID NO: 146 | — |
| 799 | isomiR Example 1 of SEQ ID NO: 147 | — |
| 800 | isomiR Example 2 of SEQ ID NO: 147 | — |
| 801 | isomiR Example 1 of SEQ ID NO: 148 | — |
| 802 | isomiR Example 1 of SEQ ID NO: 149 | — |
| 803 | isomiR Example 1 of SEQ ID NO: 150 | — |
| 804 | isomiR Example 2 of SEQ ID NO: 150 | — |
| 805 | isomiR Example 1 of SEQ ID NO: 151 | — |
| 806 | isomiR Example 2 of SEQ ID NO: 151 | — |
| 807 | isomiR Example 1 of SEQ ID NO: 162 | — |
| 808 | isomiR Example 1 of SEQ ID NO: 163 | — |
| 809 | isomiR Example 2 of SEQ ID NO: 163 | — |
| 810 | isomiR Example 1 of SEQ ID NO: 164 | — |
| 811 | isomiR Example 2 of SEQ ID NO: 164 | — |
| 812 | isomiR Example 1 of SEQ ID NO: 167 | — |
| 813 | isomiR Example 2 of SEQ ID NO: 167 | — |
| 814 | isomiR Example 1 of SEQ ID NO: 168 | — |
| 815 | isomiR Example 1 of SEQ ID NO: 170 | — |
| 816 | isomiR Example 2 of SEQ ID NO: 170 | — |
| 817 | isomiR Example 1 of SEQ ID NO: 171 | — |
| 818 | isomiR Example 2 of SEQ ID NO: 171 | — |
| 819 | isomiR Example 1 of SEQ ID NO: 175 | — |
| 820 | isomiR Example 2 of SEQ ID NO: 175 | — |
| 821 | isomiR Example 1 of SEQ ID NO: 177 | — |
| 822 | isomiR Example 2 of SEQ ID NO: 177 | — |
| 823 | isomiR Example 1 of SEQ ID NO: 178 | — |
| 824 | isomiR Example 1 of SEQ ID NO: 179 | — |
| 825 | isomiR Example 2 of SEQ ID NO: 179 | — |
| 826 | isomiR Example 1 of SEQ ID NO: 180 | — |
| 827 | isomiR Example 2 of SEQ ID NO: 180 | — |
| 828 | isomiR Example 1 of SEQ ID NO: 181 | — |
| 829 | isomiR Example 2 of SEQ ID NO: 181 | — |
| 830 | isomiR Example 1 of SEQ ID NO: 182 | — |
| 831 | isomiR Example 2 of SEQ ID NO: 182 | — |
| 832 | isomiR Example 1 of SEQ ID NO: 183 | — |
| 833 | isomiR Example 2 of SEQ ID NO: 183 | — |
| 834 | isomiR Example 1 of SEQ ID NO: 184 | — |
| 835 | isomiR Example 2 of SEQ ID NO: 184 | — |
| 836 | isomiR Example 1 of SEQ ID NO: 185 | — |
| 837 | isomiR Example 2 of SEQ ID NO: 185 | — |
| 838 | isomiR Example 1 of SEQ ID NO: 186 | — |
| 839 | isomiR Example 1 of SEQ ID NO: 187 | — |
| 840 | isomiR Example 1 of SEQ ID NO: 188 | — |
| 841 | isomiR Example 2 of SEQ ID NO: 188 | — |
| 842 | isomiR Example 1 of SEQ ID NO: 189 | — |
| 843 | isomiR Example 2 of SEQ ID NO: 189 | — |
| 844 | isomiR Example 1 of SEQ ID NO: 190 | — |
| 845 | isomiR Example 2 of SEQ ID NO: 190 | — |
| 846 | isomiR Example 1 of SEQ ID NO: 191 | — |
| 847 | isomiR Example 2 of SEQ ID NO: 191 | — |
| 848 | isomiR Example 1 of SEQ ID NO: 192 | — |
| 849 | isomiR Example 2 of SEQ ID NO: 192 | — |
| 850 | isomiR Example 1 of SEQ ID NO: 193 | — |
| 851 | isomiR Example 2 of SEQ ID NO: 193 | — |
| 852 | isomiR Example 1 of SEQ ID NO: 194 | — |
| 853 | isomiR Example 1 of SEQ ID NO: 195 | — |
| 854 | isomiR Example 2 of SEQ ID NO: 195 | — |
| 855 | isomiR Example 1 of SEQ ID NO: 196 | — |
| 856 | isomiR Example 2 of SEQ ID NO: 196 | — |
| 857 | isomiR Example 1 of SEQ ID NO: 197 | — |
| 858 | isomiR Example 2 of SEQ ID NO: 197 | — |
| 859 | isomiR Example 1 of SEQ ID NO: 198 | — |
| 860 | isomiR Example 2 of SEQ ID NO: 198 | — |
| 861 | isomiR Example 1 of SEQ ID NO: 199 | — |
| 862 | isomiR Example 1 of SEQ ID NO: 200 | — |
| 863 | isomiR Example 2 of SEQ ID NO: 200 | — |
| 864 | isomiR Example 1 of SEQ ID NO: 201 | — |
| 865 | isomiR Example 2 of SEQ ID NO: 201 | — |
| 866 | isomiR Example 1 of SEQ ID NO: 202 | — |
| 867 | isomiR Example 2 of SEQ ID NO: 202 | — |
| 868 | isomiR Example 1 of SEQ ID NO: 203 | — |
| 869 | isomiR Example 2 of SEQ ID NO: 203 | — |
| 870 | isomiR Example 1 of SEQ ID NO: 204 | — |
| 871 | isomiR Example 2 of SEQ ID NO: 204 | — |
| 872 | isomiR Example 1 of SEQ ID NO: 205 | — |
| 873 | isomiR Example 1 of SEQ ID NO: 206 | — |
| 874 | isomiR Example 2 of SEQ ID NO: 206 | — |
| 875 | isomiR Example 1 of SEQ ID NO: 207 | — |
| 876 | isomiR Example 2 of SEQ ID NO: 207 | — |
| 877 | isomiR Example 1 of SEQ ID NO: 209 | — |
| 878 | isomiR Example 2 of SEQ ID NO: 209 | — |
| 879 | isomiR Example 1 of SEQ ID NO: 210 | — |
| 880 | isomiR Example 2 of SEQ ID NO: 210 | — |
| 881 | isomiR Example 1 of SEQ ID NO: 211 | — |
| 882 | isomiR Example 2 of SEQ ID NO: 211 | — |
| 883 | isomiR Example 1 of SEQ ID NO: 212 | — |
| 884 | isomiR Example 2 of SEQ ID NO: 212 | — |
| 885 | isomiR Example 1 of SEQ ID NO: 213 | — |
| 886 | isomiR Example 1 of SEQ ID NO: 214 | — |
| 887 | isomiR Example 2 of SEQ ID NO: 214 | — |
| 888 | isomiR Example 1 of SEQ ID NO: 215 | — |
| 889 | isomiR Example 1 of SEQ ID NO: 216 | — |
| 890 | isomiR Example 2 of SEQ ID NO: 216 | — |
| 891 | isomiR Example 1 of SEQ ID NO: 219 | — |
| 892 | isomiR Example 1 of SEQ ID NO: 220 | — |
| 893 | isomiR Example 2 of SEQ ID NO: 220 | — |
| 894 | isomiR Example 1 of SEQ ID NO: 221 | — |
| 895 | isomiR Example 1 of SEQ ID NO: 222 | — |
| 896 | isomiR Example 1 of SEQ ID NO: 223 | — |
| 897 | isomiR Example 2 of SEQ ID NO: 223 | — |
| 898 | isomiR Example 1 of SEQ ID NO: 224 | — |
| 899 | isomiR Example 2 of SEQ ID NO: 224 | — |
| 900 | isomiR Example 1 of SEQ ID NO: 225 | — |
| 901 | isomiR Example 1 of SEQ ID NO: 226 | — |
| 902 | isomiR Example 2 of SEQ ID NO: 226 | — |
| 903 | isomiR Example 1 of SEQ ID NO: 227 | — |
| 904 | isomiR Example 1 of SEQ ID NO: 229 | — |
| 905 | isomiR Example 2 of SEQ ID NO: 229 | — |
| 906 | isomiR Example 1 of SEQ ID NO: 230 | — |
| 907 | isomiR Example 2 of SEQ ID NO: 230 | — |
| 908 | isomiR Example 1 of SEQ ID NO: 231 | — |
| 909 | isomiR Example 2 of SEQ ID NO: 231 | — |
| 910 | isomiR Example 1 of SEQ ID NO: 232 | — |
| 911 | isomiR Example 1 of SEQ ID NO: 233 | — |
| 912 | isomiR Example 1 of SEQ ID NO: 235 | — |
| 913 | isomiR Example 2 of SEQ ID NO: 235 | — |
| 914 | isomiR Example 1 of SEQ ID NO: 237 | — |
| 915 | isomiR Example 2 of SEQ ID NO: 237 | — |
| 916 | isomiR Example 1 of SEQ ID NO: 238 | — |
| 917 | isomiR Example 1 of SEQ ID NO: 240 | — |
| 918 | isomiR Example 2 of SEQ ID NO: 240 | — |
| 919 | isomiR Example 1 of SEQ ID NO: 241 | — |
| 920 | isomiR Example 2 of SEQ ID NO: 241 | — |
| 921 | isomiR Example 1 of SEQ ID NO: 242 | — |
| 922 | isomiR Example 1 of SEQ ID NO: 243 | — |
| 923 | isomiR Example 1 of SEQ ID NO: 244 | — |
| 924 | isomiR Example 2 of SEQ ID NO: 244 | — |
| 925 | isomiR Example 1 of SEQ ID NO: 245 | — |
| 926 | isomiR Example 2 of SEQ ID NO: 245 | — |
| 927 | isomiR Example 1 of SEQ ID NO: 246 | — |
| 928 | isomiR Example 2 of SEQ ID NO: 246 | — |
| 929 | isomiR Example 1 of SEQ ID NO: 248 | — |
| 930 | isomiR Example 1 of SEQ ID NO: 249 | — |
| 931 | isomiR Example 2 of SEQ ID NO: 249 | — |
| 932 | isomiR Example 1 of SEQ ID NO: 251 | — |
| 933 | isomiR Example 1 of SEQ ID NO: 252 | — |
| 934 | isomiR Example 2 of SEQ ID NO: 252 | — |
| 935 | isomiR Example 1 of SEQ ID NO: 253 | — |
| 936 | isomiR Example 2 of SEQ ID NO: 253 | — |
| 937 | isomiR Example 1 of SEQ ID NO: 254 | — |
| 938 | isomiR Example 2 of SEQ ID NO: 254 | — |
| 939 | isomiR Example 1 of SEQ ID NO: 255 | — |
| 940 | isomiR Example 1 of SEQ ID NO: 256 | — |
| 941 | isomiR Example 2 of SEQ ID NO: 256 | — |
| 942 | isomiR Example 1 of SEQ ID NO: 257 | — |
| 943 | isomiR Example 2 of SEQ ID NO: 257 | — |
| 944 | isomiR Example 1 of SEQ ID NO: 258 | — |
| 945 | isomiR Example 2 of SEQ ID NO: 258 | — |
| 946 | isomiR Example 1 of SEQ ID NO: 259 | — |
| 947 | isomiR Example 2 of SEQ ID NO: 259 | — |
| 948 | isomiR Example 1 of SEQ ID NO: 260 | — |
| 949 | isomiR Example 2 of SEQ ID NO: 260 | — |
| 950 | isomiR Example 1 of SEQ ID NO: 261 | — |
| 951 | isomiR Example 2 of SEQ ID NO: 261 | — |
| 952 | isomiR Example 1 of SEQ ID NO: 262 | — |
| 953 | isomiR Example 2 of SEQ ID NO: 262 | — |
| 954 | isomiR Example 1 of SEQ ID NO: 263 | — |
| 955 | isomiR Example 2 of SEQ ID NO: 263 | — |
| 956 | isomiR Example 1 of SEQ ID NO: 264 | — |
| 957 | isomiR Example 2 of SEQ ID NO: 264 | — |
| 958 | isomiR Example 1 of SEQ ID NO: 271 | — |
| 959 | isomiR Example 1 of SEQ ID NO: 293 | — |
| 960 | isomiR Example 1 of SEQ ID NO: 295 | — |
| 961 | isomiR Example 2 of SEQ ID NO: 295 | — |
| 962 | isomiR Example 1 of SEQ ID NO: 296 | — |
| 963 | isomiR Example 2 of SEQ ID NO: 296 | — |
| 964 | isomiR Example 1 of SEQ ID NO: 303 | — |
| 965 | isomiR Example 2 of SEQ ID NO: 303 | — |
| 966 | isomiR Example 1 of SEQ ID NO: 304 | — |
| 967 | isomiR Example 2 of SEQ ID NO: 304 | — |
| 968 | isomiR Example 1 of SEQ ID NO: 305 | — |
| 969 | isomiR Example 2 of SEQ ID NO: 305 | — |
| 970 | isomiR Example 1 of SEQ ID NO: 306 | — |
| 971 | isomiR Example 2 of SEQ ID NO: 306 | — |
| 972 | isomiR Example 1 of SEQ ID NO: 307 | — |
| 973 | isomiR Example 2 of SEQ ID NO: 307 | — |
| 974 | isomiR Example 1 of SEQ ID NO: 308 | — |
| 975 | isomiR Example 2 of SEQ ID NO: 308 | — |
| 976 | isomiR Example 1 of SEQ ID NO: 309 | — |
| 977 | isomiR Example 2 of SEQ ID NO: 309 | — |
| 978 | isomiR Example 1 of SEQ ID NO: 311 | — |
| 979 | isomiR Example 2 of SEQ ID NO: 311 | — |
| 980 | isomiR Example 1 of SEQ ID NO: 312 | — |
| 981 | isomiR Example 2 of SEQ ID NO: 312 | — |
| 982 | isomiR Example 1 of SEQ ID NO: 313 | — |
| 983 | isomiR Example 2 of SEQ ID NO: 313 | — |
| 984 | isomiR Example 1 of SEQ ID NO: 314 | — |
| 985 | isomiR Example 1 of SEQ ID NO: 315 | — |
| 986 | isomiR Example 2 of SEQ ID NO: 315 | — |
| 987 | isomiR Example 1 of SEQ ID NO: 316 | — |
| 988 | isomiR Example 2 of SEQ ID NO: 316 | — |
| 989 | isomiR Example 1 of SEQ ID NO: 317 | — |
| 990 | isomiR Example 2 of SEQ ID NO: 317 | — |
| 991 | isomiR Example 1 of SEQ ID NO: 318 | — |
| 992 | isomiR Example 2 of SEQ ID NO: 318 | — |
| 993 | isomiR Example 1 of SEQ ID NO: 319 | — |
| 994 | isomiR Example 1 of SEQ ID NO: 320 | — |
| 995 | isomiR Example 2 of SEQ ID NO: 320 | — |
| 996 | isomiR Example 1 of SEQ ID NO: 321 | — |
| 997 | isomiR Example 2 of SEQ ID NO: 321 | — |
| 998 | isomiR Example 1 of SEQ ID NO: 328 | — |
| 999 | isomiR Example 1 of SEQ ID NO: 329 | — |
| 1000 | isomiR Example 2 of SEQ ID NO: 329 | — |

### Effect of Invention

According to the present invention, lung cancer can be detected easily and in high accuracy. For example, the presence or absence of lung cancer in patients can be easily detected by using, as indicators, the determined expression levels of one to several miRNAs in blood, serum, and/or plasma of the patients, which can be collected with limited invasiveness.

The present specification encompasses the contents disclosed in Japanese Patent Application No. 2017-126933 from which the present application claims priority.

### BRIEF DESCRIPTION OF THE DRAWINGS

FIG. 1 This figure shows the relationship between the nucleotide sequences of hsa-miR-4433a-5p represented by SEQ ID NO: 142 and hsa-miR-4433a-3p represented by SEQ ID NO: 46, which are produced from the precursor hsa-mir-4433a represented by SEQ ID NO: 382.

FIG. 2 Upper panel: a discriminant (0.960×hsa-miR-1343-3p−0.703×hsa-miR-197-5p−0.184×hsa-miR-6741-5p+0.506×hsa-miR-4687-3p−0.471×hsa-miR-1268b−1.273) was prepared by use of Fisher's discriminant analysis from the measured expression level values of hsa-miR-1343-3p (SEQ ID NO: 164), hsa-miR-197-5p (SEQ ID NO: 18), hsa-miR-6741-5p (SEQ ID NO: 268), hsa-miR-4687-3p (SEQ ID NO: 147), and hsa-miR-1268b (SEQ ID NO: 184) in sera of test subjects without lung cancer (total 2,777 people including 1,233 healthy subjects, 263 benign bone and soft tissue tumor patients and benign breast disease patients, 1,281 patients having a cancer other than lung cancer) and lung cancer patients (1,186 people) selected as a training cohort, and discriminant scores obtained from the discriminant were plotted on the ordinate against the sample groups on the abscissa. In consideration of easy viewability of the figure, the discriminant scores are shown as to 400 people each extracted at random from the test subjects without lung cancer and the patients with lung cancer. The dotted line in the panel depicts a discriminant boundary that offered a discriminant score of 0 and discriminated between the two groups. Lower panel: discriminant scores obtained from assignment to the discriminant (0.960×hsa-miR-1343-3p−0.703×hsa-miR-197-5p−0.184×hsa-miR-6741-5p+0.506×hsa-miR-4687-3p−0.471×hsa-miR-1268b−1.273) prepared from the training cohort as to the measured expression level values of hsa-miR-1343-3p (SEQ ID NO: 164), hsa-miR-197-5p (SEQ ID NO: 18), hsa-miR-6741-5p (SEQ ID NO: 268), hsa-miR-4687-3p (SEQ ID NO: 147), and hsa-miR-1268b (SEQ ID NO: 184) in sera of test subjects without lung cancer (total 1,191 people including 567 healthy subjects, 105 benign bone and soft tissue tumor patients and benign breast disease patients, 519 patients having a cancer other than lung cancer) and lung cancer patients (508 people) selected as a validation cohort were plotted on the ordinate against the sample groups on the abscissa. In consideration of easy viewability of the figure, the discriminant scores are shown as to 400 people each extracted at random from the test subjects without lung cancer and the patients with lung cancer. The dotted line in the panel depicts the discriminant boundary that offered a discriminant score of 0 and discriminated between the two groups.

FIG. 3 Upper panel: the discriminant scores of the validation cohort described above are categorized on the basis of the healthy subjects and each histological type of lung cancer (small cell carcinoma, adenocarcinoma, squamous cell carcinoma, large cell carcinoma, and other lung cancers). The dotted line in the panel depicts the discriminant boundary that offered a discriminant score of 0 and discriminated between the two groups. Lower panel: the discriminant scores of the validation cohort described above are categorized on the basis of the healthy subjects and the progressive stages of lung cancer (stage I, stage II, and stage III/IV (stage III and stage IV)). The dotted line in the panel depicts the discriminant boundary that offered a discriminant score of 0 and discriminated between the two groups.

### MODES FOR CARRYING OUT THE INVENTION

Hereinafter, the present invention will be further described in detail.

### 1. Target Nucleic Acid for Lung Cancer

Primary target nucleic acids, as lung cancer markers, for detecting the presence and/or absence of lung cancer or lung cancer cells using the nucleic acids such as the nucleic acid probes or the primers for detection of lung cancer defined above according to the present invention comprise at least one miRNA selected from the group consisting of the following miRNAs: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p, or a polynucleotide complementary to the miRNA. Furthermore, at least one miRNA selected from the group consisting of the following other lung cancer markers that can be combined with these miRNAs, i.e., miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, or a polynucleotide complementary to the miRNA can also be preferably used as target nucleic acids.

These miRNAs include, for example, a human gene comprising a nucleotide sequence represented by any of SEQ ID NOs: 1 to 329 (i.e., miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, miR-92b-3p, miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, respectively), a congener thereof, a transcript thereof, or/and a variant or a derivative thereof. In this context, the gene, the congener, the transcript, the variant, and the derivative are as defined above.

The target nucleic acid is preferably a human gene comprising a nucleotide sequence represented by any of SEQ ID NOs: 1 to 329 or a transcript thereof, more preferably the transcript, i.e., a miRNA or its precursor RNA, pri-miRNA or pre-miRNA, or a polynucleotide complementary thereto.
The first target gene is the hsa-miR-6787-5p gene, a congener thereof, a transcript thereof, or a variant or a derivative thereof. None of the previously known report shows that change in the expression of the gene or the transcript thereof can serve as a marker for lung cancer.
The second target gene is the hsa-miR-920 gene, a congener thereof, a transcript thereof, or a variant or a derivative thereof. None of the previously known report shows that change in the expression of the gene or the transcript thereof can serve as a marker for lung cancer.
The third target gene is the hsa-miR-3622a-5p gene, a congener thereof, a transcript thereof, or a variant or a derivative thereof. None of the previously known report shows that change in the expression of the gene or the transcript thereof can serve as a marker for lung cancer.
The fourth target gene is the hsa-miR-1185-1-3p gene, a congener thereof, a transcript thereof, or a variant or a derivative thereof. None of the previously known report shows that change in the expression of the gene or the transcript thereof can serve as a marker for lung cancer.
The fifth target gene is the hsa-miR-4327 gene, a congener thereof, a transcript thereof, or a variant or a derivative thereof. None of the previously known report shows that change in the expression of the gene or the transcript thereof can serve as a marker for lung cancer.
The sixth target gene is the hsa-miR-5739 gene, a congener thereof, a transcript thereof, or a variant or a derivative thereof. None of the previously known report shows that change in the expression of the gene or the transcript thereof can serve as a marker for lung cancer.
The seventh target gene is the hsa-miR-937-5p gene, a congener thereof, a transcript thereof, or a variant or a derivative thereof. None of the previously known report shows that change in the expression of the gene or the transcript thereof can serve as a marker for lung cancer.
The eighth target gene is the hsa-miR-1181 gene, a congener thereof, a transcript thereof, or a variant or a derivative thereof. None of the previously known report shows that change in the expression of the gene or the transcript thereof can serve as a marker for lung cancer.
The ninth target gene is the hsa-miR-1185-2-3p gene, a congener thereof, a transcript thereof, or a variant or a derivative thereof. None of the previously known report shows that change in the expression of the gene or the transcript thereof can serve as a marker for lung cancer.
The 10th target gene is the hsa-miR-1193 gene, a congener thereof, a transcript thereof, or a variant or a derivative thereof. None of the previously known report shows that change in the expression of the gene or the transcript thereof can serve as a marker for lung cancer.
The 11th target gene is the hsa-miR-1207-5p gene, a congener thereof, a transcript thereof, or a variant or a derivative thereof. None of the previously known report shows that change in the expression of the gene or the transcript thereof can serve as a marker for lung cancer.
The 12th target gene is the hsa-miR-1238-5p gene, a congener thereof, a transcript thereof, or a variant or a derivative thereof. None of the previously known report shows that change in the expression of the gene or the transcript thereof can serve as a marker for lung
The 13th target gene is the hsa-miR-1246 gene, a congener thereof, a transcript thereof, or a variant or a derivative thereof. None of the previously known report shows that change in the expression of the gene or the transcript thereof can serve as a marker for lung cancer.
The 14th target gene is the hsa-miR-1249-5p gene, a congener thereof, a transcript thereof, or a variant or a derivative thereof. None of the previously known report shows that change in the expression of the gene or the transcript thereof can serve as a marker for lung cancer.
The 15th target gene is the hsa-miR-1292-3p gene, a congener thereof, a transcript thereof, or a variant or a derivative thereof. None of the previously known report shows that change in the expression of the gene or the transcript thereof can serve as a marker for lung cancer.
The 16th target gene is the hsa-miR-1469 gene, a congener thereof, a transcript thereof, or a variant or a derivative thereof. None of the previously known report shows that change in the expression of the gene or the transcript thereof can serve as a marker for lung cancer.
The 17th target gene is the hsa-miR-1470 gene, a congener thereof, a transcript thereof, or a variant or a derivative thereof. None of the previously known report shows that change in the expression of the gene or the transcript thereof can serve as a marker for lung cancer.
The 18th target gene is the hsa-miR-197-5p gene, a congener thereof, a transcript thereof, or a variant or a derivative thereof. None of the previously known report shows that change in the expression of the gene or the transcript thereof can serve as a marker for lung
The 19th target gene is the hsa-miR-208a-5p gene, a congener thereof, a transcript thereof, or a variant or a derivative thereof. None of the previously known report shows that change in the expression of the gene or the transcript thereof can serve as a marker for lung cancer.
The 20th target gene is the hsa-miR-2110 gene, a congener thereof, a transcript thereof, or a variant or a derivative thereof. None of the previously known report shows that change in the expression of the gene or the transcript thereof can serve as a marker for lung cancer.
The 21st target gene is the hsa-miR-211-3p gene, a congener thereof, a transcript thereof, or a variant or a derivative thereof. None of the previously known report shows that change in the expression of the gene or the transcript thereof can serve as a marker for lung cancer.
The 22nd target gene is the hsa-miR-2467-3p gene, a congener thereof, a transcript thereof, or a variant or a derivative thereof. None of the previously known report shows that change in the expression of the gene or the transcript thereof can serve as a marker for lung cancer.
The 23rd target gene is the hsa-miR-3122 gene, a congener thereof, a transcript thereof, or a variant or a derivative thereof. None of the previously known report shows that change in the expression of the gene or the transcript thereof can serve as a marker for lung cancer.
The 24th target gene is the hsa-miR-3141 gene, a congener thereof, a transcript thereof, or a variant or a derivative thereof. None of the previously known report shows that change in the expression of the gene or the transcript thereof can serve as a marker for lung cancer.
The 25th target gene is the hsa-miR-3156-5p gene, a congener thereof, a transcript thereof, or a variant or a derivative thereof. None of the previously known report shows that change in the expression of the gene or the transcript thereof can serve as a marker for lung cancer.
The 26th target gene is the hsa-miR-3158-5p gene, a congener thereof, a transcript thereof, or a variant or a derivative thereof. None of the previously known report shows that change in the expression of the gene or the transcript thereof can serve as a marker for lung cancer.
The 27th target gene is the hsa-miR-3160-5p gene, a congener thereof, a transcript thereof, or a variant or a derivative thereof. None of the previously known report shows that change in the expression of the gene or the transcript thereof can serve as a marker for lung cancer.
The 28th target gene is the hsa-miR-3180-3p gene, a congener thereof, a transcript thereof, or a variant or a derivative thereof. None of the previously known report shows that change in the expression of the gene or the transcript thereof can serve as a marker for lung cancer.
The 29th target gene is the hsa-miR-3191-3p gene, a congener thereof, a transcript thereof, or a variant or a derivative thereof. None of the previously known report shows that change in the expression of the gene or the transcript thereof can serve as a marker for lung cancer.
The 30th target gene is the hsa-miR-3194-3p gene, a congener thereof, a transcript thereof, or a variant or a derivative thereof. None of the previously known report shows that change in the expression of the gene or the transcript thereof can serve as a marker for lung
The 31st target gene is the hsa-miR-320b gene, a congener thereof, a transcript thereof, or a variant or a derivative thereof. None of the previously known report shows that change in the expression of the gene or the transcript thereof can serve as a marker for lung cancer.
The 32nd target gene is the hsa-miR-328-5p gene, a congener thereof, a transcript thereof, or a variant or a derivative thereof. None of the previously known report shows that change in the expression of the gene or the transcript thereof can serve as a marker for lung cancer.
The 33rd target gene is the hsa-miR-3610 gene, a congener thereof, a transcript thereof, or a variant or a derivative thereof. None of the previously known report shows that change in the expression of the gene or the transcript thereof can serve as a marker for lung cancer.
The 34th target gene is the hsa-miR-3619-3p gene, a congener thereof, a transcript thereof, or a variant or a derivative thereof. None of the previously known report shows that change in the expression of the gene or the transcript thereof can serve as a marker for lung cancer.
The 35th target gene is the hsa-miR-3620-5p gene, a congener thereof, a transcript thereof, or a variant or a derivative thereof. None of the previously known report shows that change in the expression of the gene or the transcript thereof can serve as a marker for lung cancer.
The 36th target gene is the hsa-miR-370-3p gene, a congener thereof, a transcript thereof, or a variant or a derivative thereof. None of the previously known report shows that change in the expression of the gene or the transcript thereof can serve as a marker for lung
The 37th target gene is the hsa-miR-373-5p gene, a congener thereof, a transcript thereof, or a variant or a derivative thereof. None of the previously known report shows that change in the expression of the gene or the transcript thereof can serve as a marker for lung cancer.
The 38th target gene is the hsa-miR-3917 gene, a congener thereof, a transcript thereof, or a variant or a derivative thereof. None of the previously known report shows that change in the expression of the gene or the transcript thereof can serve as a marker for lung cancer.
The 39th target gene is the hsa-miR-3937 gene, a congener thereof, a transcript thereof, or a variant or a derivative thereof. None of the previously known report shows that change in the expression of the gene or the transcript thereof can serve as a marker for lung cancer.
The 40th target gene is the hsa-miR-4259 gene, a congener thereof, a transcript thereof, or a variant or a derivative thereof. None of the previously known report shows that change in the expression of the gene or the transcript thereof can serve as a marker for lung cancer.
The 41st target gene is the hsa-miR-4281 gene, a congener thereof, a transcript thereof, or a variant or a derivative thereof. None of the previously known report shows that change in the expression of the gene or the transcript thereof can serve as a marker for lung cancer.
The 42nd target gene is the hsa-miR-4294 gene, a congener thereof, a transcript thereof, or a variant or a derivative thereof. None of the previously known report shows that change in the expression of the gene or the transcript thereof can serve as a marker for lung cancer.
The 43rd target gene is the hsa-miR-4419b gene, a congener thereof, a transcript thereof, or a variant or a derivative thereof. None of the previously known report shows that change in the expression of the gene or the transcript thereof can serve as a marker for lung cancer.
The 44th target gene is the hsa-miR-4428 gene, a congener thereof, a transcript thereof, or a variant or a derivative thereof. None of the previously known report shows that change in the expression of the gene or the transcript thereof can serve as a marker for lung cancer.
The 45th target gene is the hsa-miR-4429 gene, a congener thereof, a transcript thereof, or a variant or a derivative thereof. None of the previously known report shows that change in the expression of the gene or the transcript thereof can serve as a marker for lung cancer.
The 46th target gene is the hsa-miR-4433a-3p gene, a congener thereof, a transcript thereof, or a variant or a derivative thereof. None of the previously known report shows that change in the expression of the gene or the transcript thereof can serve as a marker for lung cancer.
The 47th target gene is the hsa-miR-4447 gene, a congener thereof, a transcript thereof, or a variant or a derivative thereof. None of the previously known report shows that change in the expression of the gene or the transcript thereof can serve as a marker for lung cancer.
The 48th target gene is the hsa-miR-4449 gene, a congener thereof, a transcript thereof, or a variant or a derivative thereof. None of the previously known report shows that change in the expression of the gene or the transcript thereof can serve as a marker for lung cancer.
The 49th target gene is the hsa-miR-4459 gene, a congener thereof, a transcript thereof, or a variant or a derivative thereof. None of the previously known report shows that change in the expression of the gene or the transcript thereof can serve as a marker for lung cancer.
The 50th target gene is the hsa-miR-4480 gene, a congener thereof, a transcript thereof, or a variant or a derivative thereof. None of the previously known report shows that change in the expression of the gene or the transcript thereof can serve as a marker for lung cancer.
The 51st target gene is the hsa-miR-4485-5p gene, a congener thereof, a transcript thereof, or a variant or a derivative thereof. None of the previously known report shows that change in the expression of the gene or the transcript thereof can serve as a marker for lung cancer.
The 52nd target gene is the hsa-miR-4486 gene, a congener thereof, a transcript thereof, or a variant or a derivative thereof. None of the previously known report shows that change in the expression of the gene or the transcript thereof can serve as a marker for lung cancer.
The 53rd target gene is the hsa-miR-4488 gene, a congener thereof, a transcript thereof, or a variant or a derivative thereof. None of the previously known report shows that change in the expression of the gene or the transcript thereof can serve as a marker for lung cancer.
The 54th target gene is the hsa-miR-4489 gene, a congener thereof, a transcript thereof, or a variant or a derivative thereof. None of the previously known report shows that change in the expression of the gene or the transcript thereof can serve as a marker for lung cancer.
The 55th target gene is the hsa-miR-4505 gene, a congener thereof, a transcript thereof, or a variant or a derivative thereof. None of the previously known report shows that change in the expression of the gene or the transcript thereof can serve as a marker for lung cancer.
The 56th target gene is the hsa-miR-4513 gene, a congener thereof, a transcript thereof, or a variant or a derivative thereof. None of the previously known report shows that change in the expression of the gene or the transcript thereof can serve as a marker for lung cancer.
The 57th target gene is the hsa-miR-4515 gene, a congener thereof, a transcript thereof, or a variant or a derivative thereof. None of the previously known report shows that change in the expression of the gene or the transcript thereof can serve as a marker for lung cancer.
The 58th target gene is the hsa-miR-4530 gene, a congener thereof, a transcript thereof, or a variant or a derivative thereof. None of the previously known report shows that change in the expression of the gene or the transcript thereof can serve as a marker for lung cancer.
The 59th target gene is the hsa-miR-4535 gene, a congener thereof, a transcript thereof, or a variant or a derivative thereof. None of the previously known report shows that change in the expression of the gene or the transcript thereof can serve as a marker for lung cancer.
The 60th target gene is the hsa-miR-4635 gene, a congener thereof, a transcript thereof, or a variant or a derivative thereof. None of the previously known report shows that change in the expression of the gene or the transcript thereof can serve as a marker for lung cancer.
The 61st target gene is the hsa-miR-4640-5p gene, a congener thereof, a transcript thereof, or a variant or a derivative thereof. None of the previously known report shows that change in the expression of the gene or the transcript thereof can serve as a marker for lung cancer.
The 62nd target gene is the hsa-miR-4646-5p gene, a congener thereof, a transcript thereof, or a variant or a derivative thereof. None of the previously known report shows that change in the expression of the gene or the transcript thereof can serve as a marker for lung cancer.
The 63rd target gene is the hsa-miR-4656 gene, a congener thereof, a transcript thereof, or a variant or a derivative thereof. None of the previously known report shows that change in the expression of the gene or the transcript thereof can serve as a marker for lung cancer.
The 64th target gene is the hsa-miR-4663 gene, a congener thereof, a transcript thereof, or a variant or a derivative thereof. None of the previously known report shows that change in the expression of the gene or the transcript thereof can serve as a marker for lung cancer.
The 65th target gene is the hsa-miR-4665-5p gene, a congener thereof, a transcript thereof, or a variant or a derivative thereof. None of the previously known report shows that change in the expression of the gene or the transcript thereof can serve as a marker for lung cancer.
The 66th target gene is the hsa-miR-4706 gene, a congener thereof, a transcript thereof, or a variant or a derivative thereof. None of the previously known report shows that change in the expression of the gene or the transcript thereof can serve as a marker for lung cancer.
The 67th target gene is the hsa-miR-4707-5p gene, a congener thereof, a transcript thereof, or a variant or a derivative thereof. None of the previously known report shows that change in the expression of the gene or the transcript thereof can serve as a marker for lung cancer.
The 68th target gene is the hsa-miR-4708-3p gene, a congener thereof, a transcript thereof, or a variant or a derivative thereof. None of the previously known report shows that change in the expression of the gene or the transcript thereof can serve as a marker for lung cancer.
The 69th target gene is the hsa-miR-4710 gene, a congener thereof, a transcript thereof, or a variant or a derivative thereof. None of the previously known report shows that change in the expression of the gene or the transcript thereof can serve as a marker for lung cancer.
The 70th target gene is the hsa-miR-4718 gene, a congener thereof, a transcript thereof, or a variant or a derivative thereof. None of the previously known report shows that change in the expression of the gene or the transcript thereof can serve as a marker for lung cancer.
The 71st target gene is the hsa-miR-4722-5p gene, a congener thereof, a transcript thereof, or a variant or a derivative thereof. None of the previously known report shows that change in the expression of the gene or the transcript thereof can serve as a marker for lung cancer.
The 72nd target gene is the hsa-miR-4727-3p gene, a congener thereof, a transcript thereof, or a variant or a derivative thereof. None of the previously known report shows that change in the expression of the gene or the transcript thereof can serve as a marker for lung cancer.
The 73rd target gene is the hsa-miR-4730 gene, a congener thereof, a transcript thereof, or a variant or a derivative thereof. None of the previously known report shows that change in the expression of the gene or the transcript thereof can serve as a marker for lung cancer.
The 74th target gene is the hsa-miR-4734 gene, a congener thereof, a transcript thereof, or a variant or a derivative thereof. None of the previously known report shows that change in the expression of the gene or the transcript thereof can serve as a marker for lung cancer.
The 75th target gene is the hsa-miR-4740-5p gene, a congener thereof, a transcript thereof, or a variant or a derivative thereof. None of the previously known report shows that change in the expression of the gene or the transcript thereof can serve as a marker for lung cancer.
The 76th target gene is the hsa-miR-4747-3p gene, a congener thereof, a transcript thereof, or a variant or a derivative thereof. None of the previously known report shows that change in the expression of the gene or the transcript thereof can serve as a marker for lung
The 77th target gene is the hsa-miR-4749-5p gene, a congener thereof, a transcript thereof, or a variant or a derivative thereof. None of the previously known report shows that change in the expression of the gene or the transcript thereof can serve as a marker for lung cancer.
The 78th target gene is the hsa-miR-4755-3p gene, a congener thereof, a transcript thereof, or a variant or a derivative thereof. None of the previously known report shows that change in the expression of the gene or the transcript thereof can serve as a marker for lung cancer.
The 79th target gene is the hsa-miR-4763-5p gene, a congener thereof, a transcript thereof, or a variant or a derivative thereof. None of the previously known report shows that change in the expression of the gene or the transcript thereof can serve as a marker for lung cancer.
The 80th target gene is the hsa-miR-4787-3p gene, a congener thereof, a transcript thereof, or a variant or a derivative thereof. None of the previously known report shows that change in the expression of the gene or the transcript thereof can serve as a marker for lung cancer.
The 81st target gene is the hsa-miR-5008-5p gene, a congener thereof, a transcript thereof, or a variant or a derivative thereof. None of the previously known report shows that change in the expression of the gene or the transcript thereof can serve as a marker for lung cancer.
The 82nd target gene is the hsa-miR-5010-5p gene, a congener thereof, a transcript thereof, or a variant or a derivative thereof. None of the previously known report shows that change in the expression of the gene or the transcript thereof can serve as a marker for lung
The 83rd target gene is the hsa-miR-504-3p gene, a congener thereof, a transcript thereof, or a variant or a derivative thereof. None of the previously known report shows that change in the expression of the gene or the transcript thereof can serve as a marker for lung cancer.
The 84th target gene is the hsa-miR-5090 gene, a congener thereof, a transcript thereof, or a variant or a derivative thereof. None of the previously known report shows that change in the expression of the gene or the transcript thereof can serve as a marker for lung cancer.
The 85th target gene is the hsa-miR-5100 gene, a congener thereof, a transcript thereof, or a variant or a derivative thereof. None of the previously known report shows that change in the expression of the gene or the transcript thereof can serve as a marker for lung cancer.
The 86th target gene is the hsa-miR-5196-5p gene, a congener thereof, a transcript thereof, or a variant or a derivative thereof. None of the previously known report shows that change in the expression of the gene or the transcript thereof can serve as a marker for lung cancer.
The 87th target gene is the hsa-miR-551b-5p gene, a congener thereof, a transcript thereof, or a variant or a derivative thereof. None of the previously known report shows that change in the expression of the gene or the transcript thereof can serve as a marker for lung cancer.
The 88th target gene is the hsa-miR-557 gene, a congener thereof, a transcript thereof, or a variant or a derivative thereof. None of the previously known report shows that change in the expression of the gene or the transcript thereof can serve as a marker for lung cancer.
The 89th target gene is the hsa-miR-5787 gene, a congener thereof, a transcript thereof, or a variant or a derivative thereof. None of the previously known report shows that change in the expression of the gene or the transcript thereof can serve as a marker for lung cancer.
The 90th target gene is the hsa-miR-6090 gene, a congener thereof, a transcript thereof, or a variant or a derivative thereof. None of the previously known report shows that change in the expression of the gene or the transcript thereof can serve as a marker for lung cancer.
The 91st target gene is the hsa-miR-6124 gene, a congener thereof, a transcript thereof, or a variant or a derivative thereof. None of the previously known report shows that change in the expression of the gene or the transcript thereof can serve as a marker for lung cancer.
The 92nd target gene is the hsa-miR-6132 gene, a congener thereof, a transcript thereof, or a variant or a derivative thereof. None of the previously known report shows that change in the expression of the gene or the transcript thereof can serve as a marker for lung cancer.
The 93rd target gene is the hsa-miR-6510-5p gene, a congener thereof, a transcript thereof, or a variant or a derivative thereof. None of the previously known report shows that change in the expression of the gene or the transcript thereof can serve as a marker for lung cancer.
The 94th target gene is the hsa-miR-6511b-5p gene, a congener thereof, a transcript thereof, or a variant or a derivative thereof. None of the previously known report shows that change in the expression of the gene or the transcript thereof can serve as a marker for lung cancer.
The 95th target gene is the hsa-miR-6515-3p gene, a congener thereof, a transcript thereof, or a variant or a derivative thereof. None of the previously known report shows that change in the expression of the gene or the transcript thereof can serve as a marker for lung cancer.
The 96th target gene is the hsa-miR-654-5p gene, a congener thereof, a transcript thereof, or a variant or a derivative thereof. None of the previously known report shows that change in the expression of the gene or the transcript thereof can serve as a marker for lung cancer.
The 97th target gene is the hsa-miR-658 gene, a congener thereof, a transcript thereof, or a variant or a derivative thereof. None of the previously known report shows that change in the expression of the gene or the transcript thereof can serve as a marker for lung cancer.
The 98th target gene is the hsa-miR-668-5p gene, a congener thereof, a transcript thereof, or a variant or a derivative thereof. None of the previously known report shows that change in the expression of the gene or the transcript thereof can serve as a marker for lung cancer.
The 99th target gene is the hsa-miR-6722-5p gene, a congener thereof, a transcript thereof, or a variant or a derivative thereof. None of the previously known report shows that change in the expression of the gene or the transcript thereof can serve as a marker for lung cancer.
The 100th target gene is the hsa-miR-6724-5p gene, a congener thereof, a transcript thereof, or a variant or a derivative thereof. None of the previously known report shows that change in the expression of the gene or the transcript thereof can serve as a marker for lung cancer.
The 101st target gene is the hsa-miR-6729-3p gene, a congener thereof, a transcript thereof, or a variant or a derivative thereof. None of the previously known report shows that change in the expression of the gene or the transcript thereof can serve as a marker for lung cancer.
The 102nd target gene is the hsa-miR-6737-5p gene, a congener thereof, a transcript thereof, or a variant or a derivative thereof. None of the previously known report shows that change in the expression of the gene or the transcript thereof can serve as a marker for lung cancer.
The 103rd target gene is the hsa-miR-6756-5p gene, a congener thereof, a transcript thereof, or a variant or a derivative thereof. None of the previously known report shows that change in the expression of the gene or the transcript thereof can serve as a marker for lung cancer.
The 104th target gene is the hsa-miR-6762-5p gene, a congener thereof, a transcript thereof, or a variant or a derivative thereof. None of the previously known report shows that change in the expression of the gene or the transcript thereof can serve as a marker for lung cancer.
The 105th target gene is the hsa-miR-6763-3p gene, a congener thereof, a transcript thereof, or a variant or a derivative thereof. None of the previously known report shows that change in the expression of the gene or the transcript thereof can serve as a marker for lung cancer.
The 106th target gene is the hsa-miR-6766-5p gene, a congener thereof, a transcript thereof, or a variant or a derivative thereof. None of the previously known report shows that change in the expression of the gene or the transcript thereof can serve as a marker for lung
The 107th target gene is the hsa-miR-6769a-5p gene, a congener thereof, a transcript thereof, or a variant or a derivative thereof. None of the previously known report shows that change in the expression of the gene or the transcript thereof can serve as a marker for lung cancer.
The 108th target gene is the hsa-miR-6771-5p gene, a congener thereof, a transcript thereof, or a variant or a derivative thereof. None of the previously known report shows that change in the expression of the gene or the transcript thereof can serve as a marker for lung cancer.
The 109th target gene is the hsa-miR-6786-5p gene, a congener thereof, a transcript thereof, or a variant or a derivative thereof. None of the previously known report shows that change in the expression of the gene or the transcript thereof can serve as a marker for lung cancer.
The 110th target gene is the hsa-miR-6789-5p gene, a congener thereof, a transcript thereof, or a variant or a derivative thereof. None of the previously known report shows that change in the expression of the gene or the transcript thereof can serve as a marker for lung cancer.
The 111th target gene is the hsa-miR-6794-5p gene, a congener thereof, a transcript thereof, or a variant or a derivative thereof. None of the previously known report shows that change in the expression of the gene or the transcript thereof can serve as a marker for lung cancer.
The 112th target gene is the hsa-miR-6796-3p gene, a congener thereof, a transcript thereof, or a variant or a derivative thereof. None of the previously known report shows that change in the expression of the gene or the transcript thereof can serve as a marker for lung
The 113th target gene is the hsa-miR-6797-5p gene, a congener thereof, a transcript thereof, or a variant or a derivative thereof. None of the previously known report shows that change in the expression of the gene or the transcript thereof can serve as a marker for lung cancer.
The 114th target gene is the hsa-miR-6800-3p gene, a congener thereof, a transcript thereof, or a variant or a derivative thereof. None of the previously known report shows that change in the expression of the gene or the transcript thereof can serve as a marker for lung cancer.
The 115th target gene is the hsa-miR-6802-5p gene, a congener thereof, a transcript thereof, or a variant or a derivative thereof. None of the previously known report shows that change in the expression of the gene or the transcript thereof can serve as a marker for lung cancer.
The 116th target gene is the hsa-miR-6803-5p gene, a congener thereof, a transcript thereof, or a variant or a derivative thereof. None of the previously known report shows that change in the expression of the gene or the transcript thereof can serve as a marker for lung cancer.
The 117th target gene is the hsa-miR-6805-3p gene, a congener thereof, a transcript thereof, or a variant or a derivative thereof. None of the previously known report shows that change in the expression of the gene or the transcript thereof can serve as a marker for lung cancer.
The 118th target gene is the hsa-miR-6805-5p gene, a congener thereof, a transcript thereof, or a variant or a derivative thereof. None of the previously known report shows that change in the expression of the gene or the transcript thereof can serve as a marker for lung cancer.
The 119th target gene is the hsa-miR-6807-5p gene, a congener thereof, a transcript thereof, or a variant or a derivative thereof. None of the previously known report shows that change in the expression of the gene or the transcript thereof can serve as a marker for lung cancer.
The 120th target gene is the hsa-miR-6812-5p gene, a congener thereof, a transcript thereof, or a variant or a derivative thereof. None of the previously known report shows that change in the expression of the gene or the transcript thereof can serve as a marker for lung cancer.
The 121st target gene is the hsa-miR-6819-5p gene, a congener thereof, a transcript thereof, or a variant or a derivative thereof. None of the previously known report shows that change in the expression of the gene or the transcript thereof can serve as a marker for lung cancer.
The 122nd target gene is the hsa-miR-6822-5p gene, a congener thereof, a transcript thereof, or a variant or a derivative thereof. None of the previously known report shows that change in the expression of the gene or the transcript thereof can serve as a marker for lung cancer.
The 123rd target gene is the hsa-miR-6824-5p gene, a congener thereof, a transcript thereof, or a variant or a derivative thereof. None of the previously known report shows that change in the expression of the gene or the transcript thereof can serve as a marker for lung
The 124th target gene is the hsa-miR-6826-5p gene, a congener thereof, a transcript thereof, or a variant or a derivative thereof. None of the previously known report shows that change in the expression of the gene or the transcript thereof can serve as a marker for lung cancer.
The 125th target gene is the hsa-miR-6850-5p gene, a congener thereof, a transcript thereof, or a variant or a derivative thereof. None of the previously known report shows that change in the expression of the gene or the transcript thereof can serve as a marker for lung cancer.
The 126th target gene is the hsa-miR-6858-5p gene, a congener thereof, a transcript thereof, or a variant or a derivative thereof. None of the previously known report shows that change in the expression of the gene or the transcript thereof can serve as a marker for lung cancer.
The 127th target gene is the hsa-miR-6861-5p gene, a congener thereof, a transcript thereof, or a variant or a derivative thereof. None of the previously known report shows that change in the expression of the gene or the transcript thereof can serve as a marker for lung cancer.
The 128th target gene is the hsa-miR-6880-3p gene, a congener thereof, a transcript thereof, or a variant or a derivative thereof. None of the previously known report shows that change in the expression of the gene or the transcript thereof can serve as a marker for lung cancer.
The 129th target gene is the hsa-miR-7107-5p gene, a congener thereof, a transcript thereof, or a variant or a derivative thereof. None of the previously known report shows that change in the expression of the gene or the transcript thereof can serve as a marker for lung
The 130th target gene is the hsa-miR-7109-5p gene, a congener thereof, a transcript thereof, or a variant or a derivative thereof. None of the previously known report shows that change in the expression of the gene or the transcript thereof can serve as a marker for lung cancer.
The 131st target gene is the hsa-miR-7114-5p gene, a congener thereof, a transcript thereof, or a variant or a derivative thereof. None of the previously known report shows that change in the expression of the gene or the transcript thereof can serve as a marker for lung cancer.
The 132nd target gene is the hsa-miR-7704 gene, a congener thereof, a transcript thereof, or a variant or a derivative thereof. None of the previously known report shows that change in the expression of the gene or the transcript thereof can serve as a marker for lung cancer.
The 133rd target gene is the hsa-miR-7846-3p gene, a congener thereof, a transcript thereof, or a variant or a derivative thereof. None of the previously known report shows that change in the expression of the gene or the transcript thereof can serve as a marker for lung cancer.
The 134th target gene is the hsa-miR-8052 gene, a congener thereof, a transcript thereof, or a variant or a derivative thereof. None of the previously known report shows that change in the expression of the gene or the transcript thereof can serve as a marker for lung cancer.
The 135th target gene is the hsa-miR-8060 gene, a congener thereof, a transcript thereof, or a variant or a derivative thereof. None of the previously known report shows that change in the expression of the gene or the transcript thereof can serve as a marker for lung cancer.
The 136th target gene is the hsa-miR-8071 gene, a congener thereof, a transcript thereof, or a variant or a derivative thereof. None of the previously known report shows that change in the expression of the gene or the transcript thereof can serve as a marker for lung cancer.
The 137th target gene is the hsa-miR-8073 gene, a congener thereof, a transcript thereof, or a variant or a derivative thereof. None of the previously known report shows that change in the expression of the gene or the transcript thereof can serve as a marker for lung cancer.
The 138th target gene is the hsa-miR-874-5p gene, a congener thereof, a transcript thereof, or a variant or a derivative thereof. None of the previously known report shows that change in the expression of the gene or the transcript thereof can serve as a marker for lung cancer.
The 139th target gene is the hsa-miR-204-3p gene, a congener thereof, a transcript thereof, or a variant or a derivative thereof. None of the previously known report shows that change in the expression of the gene or the transcript thereof can serve as a marker for lung cancer.
The 140th target gene is the hsa-miR-3154 gene, a congener thereof, a transcript thereof, or a variant or a derivative thereof. None of the previously known report shows that change in the expression of the gene or the transcript thereof can serve as a marker for lung cancer.
The 141st target gene is the hsa-miR-3960 gene, a congener thereof, a transcript thereof, or a variant or a derivative thereof. None of the previously known report shows that change in the expression of the gene or the transcript thereof can serve as a marker for lung cancer.
The 142nd target gene is the hsa-miR-4433a-5p gene, a congener thereof, a transcript thereof, or a variant or a derivative thereof. None of the previously known report shows that change in the expression of the gene or the transcript thereof can serve as a marker for lung cancer.
The 143rd target gene is the hsa-miR-4455 gene, a congener thereof, a transcript thereof, or a variant or a derivative thereof. None of the previously known report shows that change in the expression of the gene or the transcript thereof can serve as a marker for lung cancer.
The 144th target gene is the hsa-miR-4462 gene, a congener thereof, a transcript thereof, or a variant or a derivative thereof. None of the previously known report shows that change in the expression of the gene or the transcript thereof can serve as a marker for lung cancer.
The 145th target gene is the hsa-miR-4476 gene, a congener thereof, a transcript thereof, or a variant or a derivative thereof. None of the previously known report shows that change in the expression of the gene or the transcript thereof can serve as a marker for lung cancer.
The 146th target gene is the hsa-miR-4508 gene, a congener thereof, a transcript thereof, or a variant or a derivative thereof. None of the previously known report shows that change in the expression of the gene or the transcript thereof can serve as a marker for lung cancer.
The 147th target gene is the hsa-miR-4687-3p gene, a congener thereof, a transcript thereof, or a variant or a derivative thereof. None of the previously known report shows that change in the expression of the gene or the transcript thereof can serve as a marker for lung cancer.
The 148th target gene is the hsa-miR-4687-5p gene, a congener thereof, a transcript thereof, or a variant or a derivative thereof. None of the previously known report shows that change in the expression of the gene or the transcript thereof can serve as a marker for lung
The 149th target gene is the hsa-miR-4732-5p gene, a congener thereof, a transcript thereof, or a variant or a derivative thereof. None of the previously known report shows that change in the expression of the gene or the transcript thereof can serve as a marker for lung cancer.
The 150th target gene is the hsa-miR-4771 gene, a congener thereof, a transcript thereof, or a variant or a derivative thereof. None of the previously known report shows that change in the expression of the gene or the transcript thereof can serve as a marker for lung cancer.
The 151st target gene is the hsa-miR-642a-3p gene, a congener thereof, a transcript thereof, or a variant or a derivative thereof. None of the previously known report shows that change in the expression of the gene or the transcript thereof can serve as a marker for lung cancer.
The 152nd target gene is the hsa-miR-6732-5p gene, a congener thereof, a transcript thereof, or a variant or a derivative thereof. None of the previously known report shows that change in the expression of the gene or the transcript thereof can serve as a marker for lung cancer.
The 153rd target gene is the hsa-miR-6760-5p gene, a congener thereof, a transcript thereof, or a variant or a derivative thereof. None of the previously known report shows that change in the expression of the gene or the transcript thereof can serve as a marker for lung cancer.
The 154th target gene is the hsa-miR-6799-5p gene, a congener thereof, a transcript thereof, or a variant or a derivative thereof. None of the previously known report shows that change in the expression of the gene or the transcript thereof can serve as a marker for lung
The 155th target gene is the hsa-miR-6820-5p gene, a congener thereof, a transcript thereof, or a variant or a derivative thereof. None of the previously known report shows that change in the expression of the gene or the transcript thereof can serve as a marker for lung cancer.
The 156th target gene is the hsa-miR-6821-5p gene, a congener thereof, a transcript thereof, or a variant or a derivative thereof. None of the previously known report shows that change in the expression of the gene or the transcript thereof can serve as a marker for lung cancer.
The 157th target gene is the hsa-miR-6829-5p gene, a congener thereof, a transcript thereof, or a variant or a derivative thereof. None of the previously known report shows that change in the expression of the gene or the transcript thereof can serve as a marker for lung cancer.
The 158th target gene is the hsa-miR-6893-5p gene, a congener thereof, a transcript thereof, or a variant or a derivative thereof. None of the previously known report shows that change in the expression of the gene or the transcript thereof can serve as a marker for lung cancer.
The 159th target gene is the hsa-miR-7108-3p gene, a congener thereof, a transcript thereof, or a variant or a derivative thereof. None of the previously known report shows that change in the expression of the gene or the transcript thereof can serve as a marker for lung cancer.
The 160th target gene is the hsa-miR-7111-5p gene, a congener thereof, a transcript thereof, or a variant or a derivative thereof. None of the previously known report shows that change in the expression of the gene or the transcript thereof can serve as a marker for lung
The 161st target gene is the hsa-miR-8089 gene, a congener thereof, a transcript thereof, or a variant or a derivative thereof. None of the previously known report shows that change in the expression of the gene or the transcript thereof can serve as a marker for lung cancer.
The 162nd target gene is the hsa-miR-885-3p gene, a congener thereof, a transcript thereof, or a variant or a derivative thereof. None of the previously known report shows that change in the expression of the gene or the transcript thereof can serve as a marker for lung cancer.
The 163rd target gene is the hsa-miR-92b-3p gene, a congener thereof, a transcript thereof, or a variant or a derivative thereof. None of the previously known report shows that change in the expression of the gene or the transcript thereof can serve as a marker for lung cancer.
The 164th target gene is the hsa-miR-1343-3p gene, a congener thereof, a transcript thereof, or a variant or a derivative thereof. The previously known report shows that change in the expression of the gene or the transcript thereof can serve as a marker for lung cancer (Patent Literature 1).
The 165th target gene is the hsa-miR-6746-5p gene, a congener thereof, a transcript thereof, or a variant or a derivative thereof. The previously known report shows that change in the expression of the gene or the transcript thereof can serve as a marker for lung cancer (Patent Literature 1).
The 166th target gene is the hsa-miR-422a gene, a congener thereof, a transcript thereof, or a variant or a derivative thereof. The previously known report shows that change in the expression of the gene or the transcript thereof can serve as a marker for lung cancer (Patent Literature 2).
The 167th target gene is the hsa-miR-187-5p gene, a congener thereof, a transcript thereof, or a variant or a derivative thereof. The previously known report shows that change in the expression of the gene or the transcript thereof can serve as a marker for lung cancer (Patent Literature 1).
The 168th target gene is the hsa-miR-4632-5p gene, a congener thereof, a transcript thereof, or a variant or a derivative thereof. The previously known report shows that change in the expression of the gene or the transcript thereof can serve as a marker for lung cancer (Patent Literature 1).
The 169th target gene is the hsa-miR-6791-5p gene, a congener thereof, a transcript thereof, or a variant or a derivative thereof. The previously known report shows that change in the expression of the gene or the transcript thereof can serve as a marker for lung cancer (Patent Literature 1).
The 170th target gene is the hsa-miR-103a-3p gene, a congener thereof, a transcript thereof, or a variant or a derivative thereof. The previously known report shows that change in the expression of the gene or the transcript thereof can serve as a marker for lung cancer (Patent Literature 3).
The 171st target gene is the hsa-miR-107 gene, a congener thereof, a transcript thereof, or a variant or a derivative thereof. The previously known report shows that change in the expression of the gene or the transcript thereof can serve as a marker for lung cancer (Patent Literature 3).
The 172nd target gene is the hsa-miR-1199-5p gene, a congener thereof, a transcript thereof, or a variant or a derivative thereof. The previously known report shows that change in the expression of the gene or the transcript thereof can serve as a marker for lung cancer (Patent Literature 1).
The 173rd target gene is the hsa-miR-1225-3p gene, a congener thereof, a transcript thereof, or a variant or a derivative thereof. The previously known report shows that change in the expression of the gene or the transcript thereof can serve as a marker for lung cancer (Patent Literature 1).
The 174th target gene is the hsa-miR-1225-5p gene, a congener thereof, a transcript thereof, or a variant or a derivative thereof. The previously known report shows that change in the expression of the gene or the transcript thereof can serve as a marker for lung cancer (Patent Literature 1).
The 175th target gene is the hsa-miR-1228-5p gene, a congener thereof, a transcript thereof, or a variant or a derivative thereof. The previously known report shows that change in the expression of the gene or the transcript thereof can serve as a marker for lung cancer (Patent Literature 1).
The 176th target gene is the hsa-miR-1229-5p gene, a congener thereof, a transcript thereof, or a variant or a derivative thereof. The previously known report shows that change in the expression of the gene or the transcript thereof can serve as a marker for lung cancer (Non-Patent Literature 5).
The 177th target gene is the hsa-miR-1233-5p gene, a congener thereof, a transcript thereof, or a variant or a derivative thereof. The previously known report shows that change in the expression of the gene or the transcript thereof can serve as a marker for lung cancer
The 178th target gene is the hsa-miR-1237-5p gene, a congener thereof, a transcript thereof, or a variant or a derivative thereof. The previously known report shows that change in the expression of the gene or the transcript thereof can serve as a marker for lung cancer (Patent Literature 1).
The 179th target gene is the hsa-miR-1247-3p gene, a congener thereof, a transcript thereof, or a variant or a derivative thereof. The previously known report shows that change in the expression of the gene or the transcript thereof can serve as a marker for lung cancer (Patent Literature 1).
The 180th target gene is the hsa-miR-1249-3p gene, a congener thereof, a transcript thereof, or a variant or a derivative thereof. The previously known report shows that change in the expression of the gene or the transcript thereof can serve as a marker for lung cancer (Patent Literature 5).
The 181st target gene is the hsa-miR-1254 gene, a congener thereof, a transcript thereof, or a variant or a derivative thereof. The previously known report shows that change in the expression of the gene or the transcript thereof can serve as a marker for lung cancer (Non-Patent Literature 6).
The 182nd target gene is the hsa-miR-1260b gene, a congener thereof, a transcript thereof, or a variant or a derivative thereof. The previously known report shows that change in the expression of the gene or the transcript thereof can serve as a marker for lung cancer (Patent Literature 1).
The 183rd target gene is the hsa-miR-1268a gene, a congener thereof, a transcript thereof, or a variant or a derivative thereof. The previously known report shows that change in the expression of the gene or the transcript thereof can serve as a marker for lung cancer
The 184th target gene is the hsa-miR-1268b gene, a congener thereof, a transcript thereof, or a variant or a derivative thereof. The previously known report shows that change in the expression of the gene or the transcript thereof can serve as a marker for lung cancer (Patent Literature 1).
The 185th target gene is the hsa-miR-1273g-3p gene, a congener thereof, a transcript thereof, or a variant or a derivative thereof. The previously known report shows that change in the expression of the gene or the transcript thereof can serve as a marker for lung cancer (Patent Literature 1).
The 186th target gene is the hsa-miR-128-1-5p gene, a congener thereof, a transcript thereof, or a variant or a derivative thereof. The previously known report shows that change in the expression of the gene or the transcript thereof can serve as a marker for lung cancer (Patent Literature 1).
The 187th target gene is the hsa-miR-128-2-5p gene, a congener thereof, a transcript thereof, or a variant or a derivative thereof. The previously known report shows that change in the expression of the gene or the transcript thereof can serve as a marker for lung cancer (Patent Literature 1).
The 188th target gene is the hsa-miR-1290 gene, a congener thereof, a transcript thereof, or a variant or a derivative thereof. The previously known report shows that change in the expression of the gene or the transcript thereof can serve as a marker for lung cancer (Non-Patent Literature 1).
The 189th target gene is the hsa-miR-150-3p gene, a congener thereof, a transcript thereof, or a variant or a derivative thereof. The previously known report shows that change in the expression of the gene or the transcript thereof can serve as a marker for lung cancer (Patent Literature 3).
The 190th target gene is the hsa-miR-17-3p gene, a congener thereof, a transcript thereof, or a variant or a derivative thereof. The previously known report shows that change in the expression of the gene or the transcript thereof can serve as a marker for lung cancer (Patent Literature 1).
The 191st target gene is the hsa-miR-1908-5p gene, a congener thereof, a transcript thereof, or a variant or a derivative thereof. The previously known report shows that change in the expression of the gene or the transcript thereof can serve as a marker for lung cancer (Patent Literature 1).
The 192nd target gene is the hsa-miR-1909-3p gene, a congener thereof, a transcript thereof, or a variant or a derivative thereof. The previously known report shows that change in the expression of the gene or the transcript thereof can serve as a marker for lung cancer (Patent Literature 4).
The 193rd target gene is the hsa-miR-1914-3p gene, a congener thereof, a transcript thereof, or a variant or a derivative thereof. The previously known report shows that change in the expression of the gene or the transcript thereof can serve as a marker for lung cancer (Patent Literature 1).
The 194th target gene is the hsa-miR-1915-3p gene, a congener thereof, a transcript thereof, or a variant or a derivative thereof. The previously known report shows that change in the expression of the gene or the transcript thereof can serve as a marker for lung cancer
The 195th target gene is the hsa-miR-191-5p gene, a congener thereof, a transcript thereof, or a variant or a derivative thereof. The previously known report shows that change in the expression of the gene or the transcript thereof can serve as a marker for lung cancer (Patent Literature 1).
The 196th target gene is the hsa-miR-22-3p gene, a congener thereof, a transcript thereof, or a variant or a derivative thereof. The previously known report shows that change in the expression of the gene or the transcript thereof can serve as a marker for lung cancer (Patent Literature 1).
The 197th target gene is the hsa-miR-23b-3p gene, a congener thereof, a transcript thereof, or a variant or a derivative thereof. The previously known report shows that change in the expression of the gene or the transcript thereof can serve as a marker for lung cancer (Patent Literature 4).
The 198th target gene is the hsa-miR-24-3p gene, a congener thereof, a transcript thereof, or a variant or a derivative thereof. The previously known report shows that change in the expression of the gene or the transcript thereof can serve as a marker for lung cancer (Patent Literature 2).
The 199th target gene is the hsa-miR-296-3p gene, a congener thereof, a transcript thereof, or a variant or a derivative thereof. The previously known report shows that change in the expression of the gene or the transcript thereof can serve as a marker for lung cancer (Patent Literature 1).
The 200th target gene is the hsa-miR-296-5p gene, a congener thereof, a transcript thereof, or a variant or a derivative thereof. The previously known report shows that change in the expression of the gene or the transcript thereof can serve as a marker for lung cancer (Patent Literature 2).
The 201st target gene is the hsa-miR-3131 gene, a congener thereof, a transcript thereof, or a variant or a derivative thereof. The previously known report shows that change in the expression of the gene or the transcript thereof can serve as a marker for lung cancer (Patent Literature 1).
The 202nd target gene is the hsa-miR-3162-5p gene, a congener thereof, a transcript thereof, or a variant or a derivative thereof. The previously known report shows that change in the expression of the gene or the transcript thereof can serve as a marker for lung cancer (Patent Literature 1).
The 203rd target gene is the hsa-miR-3188 gene, a congener thereof, a transcript thereof, or a variant or a derivative thereof. The previously known report shows that change in the expression of the gene or the transcript thereof can serve as a marker for lung cancer (Patent Literature 1).
The 204th target gene is the hsa-miR-3196 gene, a congener thereof, a transcript thereof, or a variant or a derivative thereof. The previously known report shows that change in the expression of the gene or the transcript thereof can serve as a marker for lung cancer (Patent Literature 1).
The 205th target gene is the hsa-miR-3197 gene, a congener thereof, a transcript thereof, or a variant or a derivative thereof. The previously known report shows that change in the expression of the gene or the transcript thereof can serve as a marker for lung cancer (Patent Literature 1).
The 206th target gene is the hsa-miR-320a gene, a congener thereof, a transcript thereof, or a variant or a derivative thereof. The previously known report shows that change in the expression of the gene or the transcript thereof can serve as a marker for lung cancer (Patent Literature 2).
The 207th target gene is the hsa-miR-342-5p gene, a congener thereof, a transcript thereof, or a variant or a derivative thereof. The previously known report shows that change in the expression of the gene or the transcript thereof can serve as a marker for lung cancer (Non-Patent Literature 3).
The 208th target gene is the hsa-miR-3621 gene, a congener thereof, a transcript thereof, or a variant or a derivative thereof. The previously known report shows that change in the expression of the gene or the transcript thereof can serve as a marker for lung cancer (Patent Literature 1).
The 209th target gene is the hsa-miR-3648 gene, a congener thereof, a transcript thereof, or a variant or a derivative thereof. The previously known report shows that change in the expression of the gene or the transcript thereof can serve as a marker for lung cancer (Patent Literature 1).
The 210th target gene is the hsa-miR-3656 gene, a congener thereof, a transcript thereof, or a variant or a derivative thereof. The previously known report shows that change in the expression of the gene or the transcript thereof can serve as a marker for lung cancer (Patent Literature 1).
The 211th target gene is the hsa-miR-365a-5p gene, a congener thereof, a transcript thereof, or a variant or a derivative thereof. The previously known report shows that change in the expression of the gene or the transcript thereof can serve as a marker for lung cancer
The 212th target gene is the hsa-miR-3665 gene, a congener thereof, a transcript thereof, or a variant or a derivative thereof. The previously known report shows that change in the expression of the gene or the transcript thereof can serve as a marker for lung cancer (Patent Literature 1).
The 213th target gene is the hsa-miR-3679-5p gene, a congener thereof, a transcript thereof, or a variant or a derivative thereof. The previously known report shows that change in the expression of the gene or the transcript thereof can serve as a marker for lung cancer (Patent Literature 1).
The 214th target gene is the hsa-miR-371a-5p gene, a congener thereof, a transcript thereof, or a variant or a derivative thereof. The previously known report shows that change in the expression of the gene or the transcript thereof can serve as a marker for lung cancer (Patent Literature 1).
The 215th target gene is the hsa-miR-3940-5p gene, a congener thereof, a transcript thereof, or a variant or a derivative thereof. The previously known report shows that change in the expression of the gene or the transcript thereof can serve as a marker for lung cancer (Patent Literature 1).
The 216th target gene is the hsa-miR-423-5p gene, a congener thereof, a transcript thereof, or a variant or a derivative thereof. The previously known report shows that change in the expression of the gene or the transcript thereof can serve as a marker for lung cancer (Patent Literature 4).
The 217th target gene is the hsa-miR-4257 gene, a congener thereof, a transcript thereof, or a variant or a derivative thereof. The previously known report shows that change in the expression of the gene or the transcript thereof can serve as a marker for lung cancer (Patent Literature 1).
The 218th target gene is the hsa-miR-4270 gene, a congener thereof, a transcript thereof, or a variant or a derivative thereof. The previously known report shows that change in the expression of the gene or the transcript thereof can serve as a marker for lung cancer (Patent Literature 1).
The 219th target gene is the hsa-miR-4271 gene, a congener thereof, a transcript thereof, or a variant or a derivative thereof. The previously known report shows that change in the expression of the gene or the transcript thereof can serve as a marker for lung cancer (Patent Literature 1).
The 220th target gene is the hsa-miR-4286 gene, a congener thereof, a transcript thereof, or a variant or a derivative thereof. The previously known report shows that change in the expression of the gene or the transcript thereof can serve as a marker for lung cancer (Patent Literature 1).
The 221st target gene is the hsa-miR-4298 gene, a congener thereof, a transcript thereof, or a variant or a derivative thereof. The previously known report shows that change in the expression of the gene or the transcript thereof can serve as a marker for lung cancer (Patent Literature 1).
The 222nd target gene is the hsa-miR-4417 gene, a congener thereof, a transcript thereof, or a variant or a derivative thereof. The previously known report shows that change in the expression of the gene or the transcript thereof can serve as a marker for lung cancer (Patent Literature 1).
The 223rd target gene is the hsa-miR-4442 gene, a congener thereof, a transcript thereof, or a variant or a derivative thereof. The previously known report shows that change in the expression of the gene or the transcript thereof can serve as a marker for lung cancer (Patent Literature 1).
The 224th target gene is the hsa-miR-4446-3p gene, a congener thereof, a transcript thereof, or a variant or a derivative thereof. The previously known report shows that change in the expression of the gene or the transcript thereof can serve as a marker for lung cancer (Patent Literature 1).
The 225th target gene is the hsa-miR-4448 gene, a congener thereof, a transcript thereof, or a variant or a derivative thereof. The previously known report shows that change in the expression of the gene or the transcript thereof can serve as a marker for lung cancer (Patent Literature 1).
The 226th target gene is the hsa-miR-4454 gene, a congener thereof, a transcript thereof, or a variant or a derivative thereof. The previously known report shows that change in the expression of the gene or the transcript thereof can serve as a marker for lung cancer (Patent Literature 1).
The 227th target gene is the hsa-miR-4467 gene, a congener thereof, a transcript thereof, or a variant or a derivative thereof. The previously known report shows that change in the expression of the gene or the transcript thereof can serve as a marker for lung cancer (Patent Literature 1).
The 228th target gene is the hsa-miR-4472 gene, a congener thereof, a transcript thereof, or a variant or a derivative thereof. The previously known report shows that change in the expression of the gene or the transcript thereof can serve as a marker for lung cancer (Patent Literature 1).
The 229th target gene is the hsa-miR-4507 gene, a congener thereof, a transcript thereof, or a variant or a derivative thereof. The previously known report shows that change in the expression of the gene or the transcript thereof can serve as a marker for lung cancer (Patent Literature 1).
The 230th target gene is the hsa-miR-4516 gene, a congener thereof, a transcript thereof, or a variant or a derivative thereof. The previously known report shows that change in the expression of the gene or the transcript thereof can serve as a marker for lung cancer (Patent Literature 1).
The 231st target gene is the hsa-miR-451a gene, a congener thereof, a transcript thereof, or a variant or a derivative thereof. The previously known report shows that change in the expression of the gene or the transcript thereof can serve as a marker for lung cancer (Patent Literature 1).
The 232nd target gene is the hsa-miR-4649-5p gene, a congener thereof, a transcript thereof, or a variant or a derivative thereof. The previously known report shows that change in the expression of the gene or the transcript thereof can serve as a marker for lung cancer (Patent Literature 1).
The 233rd target gene is the hsa-miR-4651 gene, a congener thereof, a transcript thereof, or a variant or a derivative thereof. The previously known report shows that change in the expression of the gene or the transcript thereof can serve as a marker for lung cancer (Patent Literature 1).
The 234th target gene is the hsa-miR-4665-3p gene, a congener thereof, a transcript thereof, or a variant or a derivative thereof. The previously known report shows that change in the expression of the gene or the transcript thereof can serve as a marker for lung cancer
The 235th target gene is the hsa-miR-4674 gene, a congener thereof, a transcript thereof, or a variant or a derivative thereof. The previously known report shows that change in the expression of the gene or the transcript thereof can serve as a marker for lung cancer (Patent Literature 1).
The 236th target gene is the hsa-miR-4675 gene, a congener thereof, a transcript thereof, or a variant or a derivative thereof. The previously known report shows that change in the expression of the gene or the transcript thereof can serve as a marker for lung cancer (Patent Literature 1).
The 237th target gene is the hsa-miR-4689 gene, a congener thereof, a transcript thereof, or a variant or a derivative thereof. The previously known report shows that change in the expression of the gene or the transcript thereof can serve as a marker for lung cancer (Patent Literature 1).
The 238th target gene is the hsa-miR-4695-5p gene, a congener thereof, a transcript thereof, or a variant or a derivative thereof. The previously known report shows that change in the expression of the gene or the transcript thereof can serve as a marker for lung cancer (Patent Literature 1).
The 239th target gene is the hsa-miR-4697-5p gene, a congener thereof, a transcript thereof, or a variant or a derivative thereof. The previously known report shows that change in the expression of the gene or the transcript thereof can serve as a marker for lung cancer (Patent Literature 1).
The 240th target gene is the hsa-miR-4725-3p gene, a congener thereof, a transcript thereof, or a variant or a derivative thereof. The previously known report shows that change in the expression of the gene or the transcript thereof can serve as a marker for lung cancer (Patent Literature 1).
The 241st target gene is the hsa-miR-4739 gene, a congener thereof, a transcript thereof, or a variant or a derivative thereof. The previously known report shows that change in the expression of the gene or the transcript thereof can serve as a marker for lung cancer (Patent Literature 1).
The 242nd target gene is the hsa-miR-4745-5p gene, a congener thereof, a transcript thereof, or a variant or a derivative thereof. The previously known report shows that change in the expression of the gene or the transcript thereof can serve as a marker for lung cancer (Patent Literature 1).
The 243rd target gene is the hsa-miR-4763-3p gene, a congener thereof, a transcript thereof, or a variant or a derivative thereof. The previously known report shows that change in the expression of the gene or the transcript thereof can serve as a marker for lung cancer (Patent Literature 1).
The 244th target gene is the hsa-miR-4792 gene, a congener thereof, a transcript thereof, or a variant or a derivative thereof. The previously known report shows that change in the expression of the gene or the transcript thereof can serve as a marker for lung cancer (Patent Literature 1).
The 245th target gene is the hsa-miR-486-3p gene, a congener thereof, a transcript thereof, or a variant or a derivative thereof. The previously known report shows that change in the expression of the gene or the transcript thereof can serve as a marker for lung cancer
The 246th target gene is the hsa-miR-5001-5p gene, a congener thereof, a transcript thereof, or a variant or a derivative thereof. The previously known report shows that change in the expression of the gene or the transcript thereof can serve as a marker for lung cancer (Patent Literature 1).
The 247th target gene is the hsa-miR-5195-3p gene, a congener thereof, a transcript thereof, or a variant or a derivative thereof. The previously known report shows that change in the expression of the gene or the transcript thereof can serve as a marker for lung cancer (Patent Literature 1).
The 248th target gene is the hsa-miR-550a-5p gene, a congener thereof, a transcript thereof, or a variant or a derivative thereof. The previously known report shows that change in the expression of the gene or the transcript thereof can serve as a marker for lung cancer (Non-Patent Literature 4).
The 249th target gene is the hsa-miR-5698 gene, a congener thereof, a transcript thereof, or a variant or a derivative thereof. The previously known report shows that change in the expression of the gene or the transcript thereof can serve as a marker for lung cancer (Patent Literature 1).
The 250th target gene is the hsa-miR-6075 gene, a congener thereof, a transcript thereof, or a variant or a derivative thereof. The previously known report shows that change in the expression of the gene or the transcript thereof can serve as a marker for lung cancer (Patent Literature 1).
The 251st target gene is the hsa-miR-6088 gene, a congener thereof, a transcript thereof, or a variant or a derivative thereof. The previously known report shows that change in the expression of the gene or the transcript thereof can serve as a marker for lung cancer (Patent Literature 1).
The 252nd target gene is the hsa-miR-6089 gene, a congener thereof, a transcript thereof, or a variant or a derivative thereof. The previously known report shows that change in the expression of the gene or the transcript thereof can serve as a marker for lung cancer (Patent Literature 1).
The 253rd target gene is the hsa-miR-6125 gene, a congener thereof, a transcript thereof, or a variant or a derivative thereof. The previously known report shows that change in the expression of the gene or the transcript thereof can serve as a marker for lung cancer (Patent Literature 1).
The 254th target gene is the hsa-miR-6126 gene, a congener thereof, a transcript thereof, or a variant or a derivative thereof. The previously known report shows that change in the expression of the gene or the transcript thereof can serve as a marker for lung cancer (Patent Literature 1).
The 255th target gene is the hsa-miR-614 gene, a congener thereof, a transcript thereof, or a variant or a derivative thereof. The previously known report shows that change in the expression of the gene or the transcript thereof can serve as a marker for lung cancer (Patent Literature 1).
The 256th target gene is the hsa-miR-615-5p gene, a congener thereof, a transcript thereof, or a variant or a derivative thereof. The previously known report shows that change in the expression of the gene or the transcript thereof can serve as a marker for lung cancer (Patent Literature 1).
The 257th target gene is the hsa-miR-619-5p gene, a congener thereof, a transcript thereof, or a variant or a derivative thereof. The previously known report shows that change in the expression of the gene or the transcript thereof can serve as a marker for lung cancer (Patent Literature 1).
The 258th target gene is the hsa-miR-638 gene, a congener thereof, a transcript thereof, or a variant or a derivative thereof. The previously known report shows that change in the expression of the gene or the transcript thereof can serve as a marker for lung cancer (Patent Literature 2).
The 259th target gene is the hsa-miR-642b-3p gene, a congener thereof, a transcript thereof, or a variant or a derivative thereof. The previously known report shows that change in the expression of the gene or the transcript thereof can serve as a marker for lung cancer (Patent Literature 1).
The 260th target gene is the hsa-miR-650 gene, a congener thereof, a transcript thereof, or a variant or a derivative thereof. The previously known report shows that change in the expression of the gene or the transcript thereof can serve as a marker for lung cancer (Non-Patent Literature 2).
The 261st target gene is the hsa-miR-663a gene, a congener thereof, a transcript thereof, or a variant or a derivative thereof. The previously known report shows that change in the expression of the gene or the transcript thereof can serve as a marker for lung cancer (Patent Literature 1).
The 262nd target gene is the hsa-miR-663b gene, a congener thereof, a transcript thereof, or a variant or a derivative thereof. The previously known report shows that change in the expression of the gene or the transcript thereof can serve as a marker for lung cancer
The 263rd target gene is the hsa-miR-6717-5p gene, a congener thereof, a transcript thereof, or a variant or a derivative thereof. The previously known report shows that change in the expression of the gene or the transcript thereof can serve as a marker for lung cancer (Patent Literature 1).
The 264th target gene is the hsa-miR-6721-5p gene, a congener thereof, a transcript thereof, or a variant or a derivative thereof. The previously known report shows that change in the expression of the gene or the transcript thereof can serve as a marker for lung cancer (Patent Literature 1).
The 265th target gene is the hsa-miR-6726-5p gene, a congener thereof, a transcript thereof, or a variant or a derivative thereof. The previously known report shows that change in the expression of the gene or the transcript thereof can serve as a marker for lung cancer (Patent Literature 1).
The 266th target gene is the hsa-miR-6727-5p gene, a congener thereof, a transcript thereof, or a variant or a derivative thereof. The previously known report shows that change in the expression of the gene or the transcript thereof can serve as a marker for lung cancer (Patent Literature 1).
The 267th target gene is the hsa-miR-6738-5p gene, a congener thereof, a transcript thereof, or a variant or a derivative thereof. The previously known report shows that change in the expression of the gene or the transcript thereof can serve as a marker for lung cancer (Patent Literature 1).
The 268th target gene is the hsa-miR-6741-5p gene, a congener thereof, a transcript thereof, or a variant or a derivative thereof. The previously known report shows that change in the expression of the gene or the transcript thereof can serve as a marker for lung cancer
The 269th target gene is the hsa-miR-6749-5p gene, a congener thereof, a transcript thereof, or a variant or a derivative thereof. The previously known report shows that change in the expression of the gene or the transcript thereof can serve as a marker for lung cancer (Patent Literature 1).
The 270th target gene is the hsa-miR-6752-5p gene, a congener thereof, a transcript thereof, or a variant or a derivative thereof. The previously known report shows that change in the expression of the gene or the transcript thereof can serve as a marker for lung cancer (Patent Literature 1).
The 271st target gene is the hsa-miR-675-5p gene, a congener thereof, a transcript thereof, or a variant or a derivative thereof. The previously known report shows that change in the expression of the gene or the transcript thereof can serve as a marker for lung cancer (Patent Literature 1).
The 272nd target gene is the hsa-miR-6757-5p gene, a congener thereof, a transcript thereof, or a variant or a derivative thereof. The previously known report shows that change in the expression of the gene or the transcript thereof can serve as a marker for lung cancer (Patent Literature 1).
The 273rd target gene is the hsa-miR-6763-5p gene, a congener thereof, a transcript thereof, or a variant or a derivative thereof. The previously known report shows that change in the expression of the gene or the transcript thereof can serve as a marker for lung cancer (Patent Literature 1).
The 274th target gene is the hsa-miR-6765-5p gene, a congener thereof, a transcript thereof, or a variant or a derivative thereof. The previously known report shows that change in the expression of the gene or the transcript thereof can serve as a marker for lung cancer (Patent Literature 1).
The 275th target gene is the hsa-miR-6775-5p gene, a congener thereof, a transcript thereof, or a variant or a derivative thereof. The previously known report shows that change in the expression of the gene or the transcript thereof can serve as a marker for lung cancer (Patent Literature 1).
The 276th target gene is the hsa-miR-6780b-5p gene, a congener thereof, a transcript thereof, or a variant or a derivative thereof. The previously known report shows that change in the expression of the gene or the transcript thereof can serve as a marker for lung cancer (Patent Literature 1).
The 277th target gene is the hsa-miR-6782-5p gene, a congener thereof, a transcript thereof, or a variant or a derivative thereof. The previously known report shows that change in the expression of the gene or the transcript thereof can serve as a marker for lung cancer (Patent Literature 1).
The 278th target gene is the hsa-miR-6784-5p gene, a congener thereof, a transcript thereof, or a variant or a derivative thereof. The previously known report shows that change in the expression of the gene or the transcript thereof can serve as a marker for lung cancer (Patent Literature 1).
The 279th target gene is the hsa-miR-6800-5p gene, a congener thereof, a transcript thereof, or a variant or a derivative thereof. The previously known report shows that change in the expression of the gene or the transcript thereof can serve as a marker for lung cancer
The 280th target gene is the hsa-miR-6806-5p gene, a congener thereof, a transcript thereof, or a variant or a derivative thereof. The previously known report shows that change in the expression of the gene or the transcript thereof can serve as a marker for lung cancer (Patent Literature 1).
The 281st target gene is the hsa-miR-6840-3p gene, a congener thereof, a transcript thereof, or a variant or a derivative thereof. The previously known report shows that change in the expression of the gene or the transcript thereof can serve as a marker for lung cancer (Patent Literature 1).
The 282nd target gene is the hsa-miR-6848-5p gene, a congener thereof, a transcript thereof, or a variant or a derivative thereof. The previously known report shows that change in the expression of the gene or the transcript thereof can serve as a marker for lung cancer (Patent Literature 1).
The 283rd target gene is the hsa-miR-6851-5p gene, a congener thereof, a transcript thereof, or a variant or a derivative thereof. The previously known report shows that change in the expression of the gene or the transcript thereof can serve as a marker for lung cancer (Patent Literature 1).
The 284th target gene is the hsa-miR-6870-5p gene, a congener thereof, a transcript thereof, or a variant or a derivative thereof. The previously known report shows that change in the expression of the gene or the transcript thereof can serve as a marker for lung cancer (Patent Literature 1).
The 285th target gene is the hsa-miR-6872-3p gene, a congener thereof, a transcript thereof, or a variant or a derivative thereof. The previously known report shows that change in the expression of the gene or the transcript thereof can serve as a marker for lung cancer
The 286th target gene is the hsa-miR-6875-5p gene, a congener thereof, a transcript thereof, or a variant or a derivative thereof. The previously known report shows that change in the expression of the gene or the transcript thereof can serve as a marker for lung cancer (Patent Literature 1).
The 287th target gene is the hsa-miR-6877-5p gene, a congener thereof, a transcript thereof, or a variant or a derivative thereof. The previously known report shows that change in the expression of the gene or the transcript thereof can serve as a marker for lung cancer (Patent Literature 1).
The 288th target gene is the hsa-miR-6879-5p gene, a congener thereof, a transcript thereof, or a variant or a derivative thereof. The previously known report shows that change in the expression of the gene or the transcript thereof can serve as a marker for lung cancer (Patent Literature 1).
The 289th target gene is the hsa-miR-6880-5p gene, a congener thereof, a transcript thereof, or a variant or a derivative thereof. The previously known report shows that change in the expression of the gene or the transcript thereof can serve as a marker for lung cancer (Patent Literature 1).
The 290th target gene is the hsa-miR-6885-5p gene, a congener thereof, a transcript thereof, or a variant or a derivative thereof. The previously known report shows that change in the expression of the gene or the transcript thereof can serve as a marker for lung cancer (Patent Literature 1).
The 291st target gene is the hsa-miR-6887-5p gene, a congener thereof, a transcript thereof, or a variant or a derivative thereof. The previously known report shows that change in the expression of the gene or the transcript thereof can serve as a marker for lung cancer (Patent Literature 1).
The 292nd target gene is the hsa-miR-7108-5p gene, a congener thereof, a transcript thereof, or a variant or a derivative thereof. The previously known report shows that change in the expression of the gene or the transcript thereof can serve as a marker for lung cancer (Patent Literature 1).
The 293rd target gene is the hsa-miR-711 gene, a congener thereof, a transcript thereof, or a variant or a derivative thereof. The previously known report shows that change in the expression of the gene or the transcript thereof can serve as a marker for lung cancer (Patent Literature 1).
The 294th target gene is the hsa-miR-7113-3p gene, a congener thereof, a transcript thereof, or a variant or a derivative thereof. The previously known report shows that change in the expression of the gene or the transcript thereof can serve as a marker for lung cancer (Patent Literature 1).
The 295th target gene is the hsa-miR-744-5p gene, a congener thereof, a transcript thereof, or a variant or a derivative thereof. The previously known report shows that change in the expression of the gene or the transcript thereof can serve as a marker for lung cancer (Patent Literature 5).
The 296th target gene is the hsa-miR-760 gene, a congener thereof, a transcript thereof, or a variant or a derivative thereof. The previously known report shows that change in the expression of the gene or the transcript thereof can serve as a marker for lung cancer (Patent Literature 1).
The 297th target gene is the hsa-miR-7845-5p gene, a congener thereof, a transcript thereof, or a variant or a derivative thereof. The previously known report shows that change in the expression of the gene or the transcript thereof can serve as a marker for lung cancer (Patent Literature 1).
The 298th target gene is the hsa-miR-7847-3p gene, a congener thereof, a transcript thereof, or a variant or a derivative thereof. The previously known report shows that change in the expression of the gene or the transcript thereof can serve as a marker for lung cancer (Patent Literature 1).
The 299th target gene is the hsa-miR-7977 gene, a congener thereof, a transcript thereof, or a variant or a derivative thereof. The previously known report shows that change in the expression of the gene or the transcript thereof can serve as a marker for lung cancer (Patent Literature 1).
The 300th target gene is the hsa-miR-8059 gene, a congener thereof, a transcript thereof, or a variant or a derivative thereof. The previously known report shows that change in the expression of the gene or the transcript thereof can serve as a marker for lung cancer (Patent Literature 1).
The 301st target gene is the hsa-miR-8063 gene, a congener thereof, a transcript thereof, or a variant or a derivative thereof. The previously known report shows that change in the expression of the gene or the transcript thereof can serve as a marker for lung cancer (Patent Literature 1).
The 302nd target gene is the hsa-miR-8072 gene, a congener thereof, a transcript thereof, or a variant or a derivative thereof. The previously known report shows that change in the expression of the gene or the transcript thereof can serve as a marker for lung cancer
The 303rd target gene is the hsa-miR-874-3p gene, a congener thereof, a transcript thereof, or a variant or a derivative thereof. The previously known report shows that change in the expression of the gene or the transcript thereof can serve as a marker for lung cancer (Patent Literature 5).
The 304th target gene is the hsa-miR-92a-2-5p gene, a congener thereof, a transcript thereof, or a variant or a derivative thereof. The previously known report shows that change in the expression of the gene or the transcript thereof can serve as a marker for lung cancer (Patent Literature 1).
The 305th target gene is the hsa-miR-92b-5p gene, a congener thereof, a transcript thereof, or a variant or a derivative thereof. The previously known report shows that change in the expression of the gene or the transcript thereof can serve as a marker for lung cancer (Patent Literature 1).
The 306th target gene is the hsa-miR-940 gene, a congener thereof, a transcript thereof, or a variant or a derivative thereof. The previously known report shows that change in the expression of the gene or the transcript thereof can serve as a marker for lung cancer (Patent Literature 1).
The 307th target gene is the hsa-miR-1228-3p gene, a congener thereof, a transcript thereof, or a variant or a derivative thereof. The previously known report shows that change in the expression of the gene or the transcript thereof can serve as a marker for lung cancer (Patent Literature 1).
The 308th target gene is the hsa-miR-1275 gene, a congener thereof, a transcript thereof, or a variant or a derivative thereof. The previously known report shows that change in the expression of the gene or the transcript thereof can serve as a marker for lung cancer (Patent Literature 5).
The 309th target gene is the hsa-miR-1307-3p gene, a congener thereof, a transcript thereof, or a variant or a derivative thereof. The previously known report shows that change in the expression of the gene or the transcript thereof can serve as a marker for lung cancer (Patent Literature 1).
The 310th target gene is the hsa-miR-1343-5p gene, a congener thereof, a transcript thereof, or a variant or a derivative thereof. The previously known report shows that change in the expression of the gene or the transcript thereof can serve as a marker for lung cancer (Patent Literature 1).
The 311th target gene is the hsa-miR-23a-3p gene, a congener thereof, a transcript thereof, or a variant or a derivative thereof. The previously known report shows that change in the expression of the gene or the transcript thereof can serve as a marker for lung cancer (Patent Literature 2).
The 312th target gene is the hsa-miR-29b-3p gene, a congener thereof, a transcript thereof, or a variant or a derivative thereof. The previously known report shows that change in the expression of the gene or the transcript thereof can serve as a marker for lung cancer (Patent Literature 2).
The 313th target gene is the hsa-miR-3135b gene, a congener thereof, a transcript thereof, or a variant or a derivative thereof. The previously known report shows that change in the expression of the gene or the transcript thereof can serve as a marker for lung cancer
The 314th target gene is the hsa-miR-3185 gene, a congener thereof, a transcript thereof, or a variant or a derivative thereof. The previously known report shows that change in the expression of the gene or the transcript thereof can serve as a marker for lung cancer (Patent Literature 1).
The 315th target gene is the hsa-miR-4532 gene, a congener thereof, a transcript thereof, or a variant or a derivative thereof. The previously known report shows that change in the expression of the gene or the transcript thereof can serve as a marker for lung cancer (Patent Literature 1).
The 316th target gene is the hsa-miR-4690-5p gene, a congener thereof, a transcript thereof, or a variant or a derivative thereof. The previously known report shows that change in the expression of the gene or the transcript thereof can serve as a marker for lung cancer (Patent Literature 1).
The 317th target gene is the hsa-miR-4758-5p gene, a congener thereof, a transcript thereof, or a variant or a derivative thereof. The previously known report shows that change in the expression of the gene or the transcript thereof can serve as a marker for lung cancer (Patent Literature 1).
The 318th target gene is the hsa-miR-4783-3p gene, a congener thereof, a transcript thereof, or a variant or a derivative thereof. The previously known report shows that change in the expression of the gene or the transcript thereof can serve as a marker for lung cancer (Patent Literature 1).
The 319th target gene is the hsa-miR-6131 gene, a congener thereof, a transcript thereof, or a variant or a derivative thereof. The previously known report shows that change in the expression of the gene or the transcript thereof can serve as a marker for lung cancer (Patent Literature 1).
The 320th target gene is the hsa-miR-625-3p gene, a congener thereof, a transcript thereof, or a variant or a derivative thereof. The previously known report shows that change in the expression of the gene or the transcript thereof can serve as a marker for lung cancer (Patent Literature 4).
The 321st target gene is the hsa-miR-6511a-5p gene, a congener thereof, a transcript thereof, or a variant or a derivative thereof. The previously known report shows that change in the expression of the gene or the transcript thereof can serve as a marker for lung cancer (Patent Literature 1).
The 322nd target gene is the hsa-miR-6765-3p gene, a congener thereof, a transcript thereof, or a variant or a derivative thereof. The previously known report shows that change in the expression of the gene or the transcript thereof can serve as a marker for lung cancer (Patent Literature 1).
The 323rd target gene is the hsa-miR-6816-5p gene, a congener thereof, a transcript thereof, or a variant or a derivative thereof. The previously known report shows that change in the expression of the gene or the transcript thereof can serve as a marker for lung cancer (Patent Literature 1).
The 324th target gene is the hsa-miR-6825-5p gene, a congener thereof, a transcript thereof, or a variant or a derivative thereof. The previously known report shows that change in the expression of the gene or the transcript thereof can serve as a marker for lung cancer (Patent Literature 1).
The 325th target gene is the hsa-miR-6845-5p gene, a congener thereof, a transcript thereof, or a variant or a derivative thereof. The previously known report shows that change in the expression of the gene or the transcript thereof can serve as a marker for lung cancer (Patent Literature 1).
The 326th target gene is the hsa-miR-7150 gene, a congener thereof, a transcript thereof, or a variant or a derivative thereof. The previously known report shows that change in the expression of the gene or the transcript thereof can serve as a marker for lung cancer (Patent Literature 1).
The 327th target gene is the hsa-miR-7641 gene, a congener thereof, a transcript thereof, or a variant or a derivative thereof. The previously known report shows that change in the expression of the gene or the transcript thereof can serve as a marker for lung cancer (Patent Literature 1).
The 328th target gene is the hsa-miR-7975 gene, a congener thereof, a transcript thereof, or a variant or a derivative thereof. The previously known report shows that change in the expression of the gene or the transcript thereof can serve as a marker for lung cancer (Patent Literature 1).
The 329th target gene is the hsa-miR-92a-3p gene, a congener thereof, a transcript thereof, or a variant or a derivative thereof. The previously known report shows that change in the expression of the gene or the transcript thereof can serve as a marker for lung cancer (Patent Literature 1).

In one aspect, the present invention relates to a marker containing at least one of the target nucleic acids described above for detecting lung cancer or for diagnosing lung cancer.

In one aspect, the present invention relates to use of at least one of the target nucleic acids described above for detecting lung cancer or for diagnosing lung cancer.

### 2. Nucleic Acid for Detection of Lung Cancer

In the present invention, the nucleic acids for detecting lung cancer, e.g., nucleic acid probes or primers that can be used for diagnosing lung cancer enable qualitative and/or quantitative measurement of the presence, expression levels, or existing amounts (abundance) of: human-derived miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, miR-92b-3p, miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, as target nucleic acids for lung cancer, or combinations thereof, congeners thereof, transcripts thereof, or variants or derivatives thereof.

The expression levels of the target nucleic acids described above are increased or decreased (hereinafter, referred to as “increased/decreased”) depending on the types of the target nucleic acids in subjects having lung cancer as compared with healthy subjects, benign bone and soft tissue tumor patients and benign breast disease patients, and subjects having a cancer other than lung cancer. Hence, the kit or device of the present invention can be effectively used for measuring expression levels of the target nucleic acids in body fluids from subjects (e.g., humans) suspected of having lung cancer and body fluids from healthy subjects, benign bone and soft tissue tumor patients and benign breast disease patients (or diseased animals), and patients (or cancer animals) having a cancer other than lung cancer, and thereby detecting lung cancer through the comparison thereof.

The nucleic acid probe or primer(s) that can be used in the present invention is, for example, a nucleic acid probe capable of specifically binding to a polynucleotide consisting of a nucleotide sequence represented by at least one of SEQ ID NOs: 1 to 163; or a primer(s) for amplifying a polynucleotide consisting of a nucleotide sequence represented by at least one of SEQ ID NOs: 1 to 163.

The nucleic acid probe or primer(s) that can be used in the present invention may further comprise, for example, a nucleic acid probe capable of specifically binding to a polynucleotide consisting of a nucleotide sequence represented by at least one of SEQ ID NOs: 164 to 329; or a primer(s) for amplifying a polynucleotide consisting of a nucleotide sequence represented by at least one of SEQ ID NOs: 164 to 329.

In a preferred embodiment of the present invention, specifically, these nucleic acid probes or primers comprise a combination of one or more polynucleotides selected from: a group of polynucleotides comprising nucleotide sequences represented by any of SEQ ID NOs: 1 to 1000, or the nucleotide sequences in which the nucleic acid u is replaced with t, and a group of complementary polynucleotides thereof; a group of polynucleotides respectively hybridizing under stringent conditions (mentioned later) to DNAs consisting of nucleotide sequences complementary to these nucleotide sequences, and a group of complementary polynucleotides thereof; and a group of polynucleotides comprising 15 or more, preferably 17 or more consecutive nucleotides and being from the nucleotide sequences of these polynucleotide groups. These polynucleotides can be used as nucleic acid probes and primers for detecting the lung cancer markers as target nucleic acids.

More specifically, examples of the nucleic acid probes or the primers that can be used in the present invention include one or more polynucleotides selected from the group consisting of the following polynucleotides (a) to (e):
(a) a polynucleotide consisting of a nucleotide sequence represented by any of SEQ ID NOs: 1 to 163 or a nucleotide sequence derived from the nucleotide sequence in which the nucleic acid u is replaced with t, a variant thereof, a derivative thereof, or a fragment thereof comprising 15 or more consecutive nucleotides;
(b) a polynucleotide comprising a nucleotide sequence represented by any of SEQ ID NOs: 1 to 163;
(c) a polynucleotide consisting of a nucleotide sequence complementary to a nucleotide sequence represented by any of SEQ ID NOs: 1 to 163, or a nucleotide sequence derived from the nucleotide sequence in which the nucleic acid u is replaced with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 163, or a nucleotide sequence derived from the nucleotide sequence in which the nucleic acid u is replaced with t; and
(e) a polynucleotide hybridizing under stringent conditions to any of the polynucleotides (a) to (d).

In addition to at least one polynucleotide selected from any of the polynucleotides (a) to (e), the nucleic acid probes or the primers that can be used in the present invention may further comprise any of the following polynucleotides (f) to (j):
(f) a polynucleotide consisting of a nucleotide sequence represented by any of SEQ ID NOs: 164 to 329, or a nucleotide sequence derived from the nucleotide sequence in which the nucleic acid u is replaced with t, a variant thereof, a derivative thereof, or a fragment thereof comprising 15 or more consecutive nucleotides;
(g) a polynucleotide comprising a nucleotide sequence represented by any of SEQ ID NOs: 164 to 329;
(h) a polynucleotide consisting of a nucleotide sequence complementary to a nucleotide sequence represented by any of SEQ ID NOs: 164 to 329, or a nucleotide sequence derived from the nucleotide sequence in which the nucleic acid u is replaced with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 164 to 329, or a nucleotide sequence derived from the nucleotide sequence in which the nucleic acid u is replaced with t; and
(j) a polynucleotide hybridizing under stringent conditions to any of the polynucleotides (f) to (i).

These polynucleotides or fragments thereof used in the present invention may each be DNA or may each be RNA.

The polynucleotides that can be used in the present invention can be prepared by use of a general technique such as a DNA recombination technique, a PCR method, or a method using an automatic DNA/RNA synthesizer.

The DNA recombination technique and the PCR method may employ techniques described in, for example, Ausubel et al., Current Protocols in Molecular Biology, John Willey & Sons, US (1993); and Sambrook et al., Molecular Cloning—A Laboratory Manual, Cold Spring Harbor Laboratory Press, US (1989).

The human-derived miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, miR-92b-3p, miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p represented by SEQ ID NOs: 1 to 329 are known, and methods to obtain them are also known as mentioned above. Therefore, each polynucleotide that can be used as a nucleic acid probe or a primer in the present invention can be prepared by cloning the gene.

Such nucleic acid probes or primers can be chemically synthesized using an automatic DNA synthesizer. In general, the phosphoramidite method is used in this synthesis, and single-stranded DNA in length of up to approximately 100 nucleotides can be automatically synthesized by this method. The automatic DNA synthesizer is commercially available from, for example, Polygen GmbH, ABI, or Applied Biosystems, Inc.

Alternatively, the polynucleotides of the present invention can also be prepared by cDNA cloning methods. The cDNA cloning technique may employ, for example, microRNA Cloning Kit Wako.

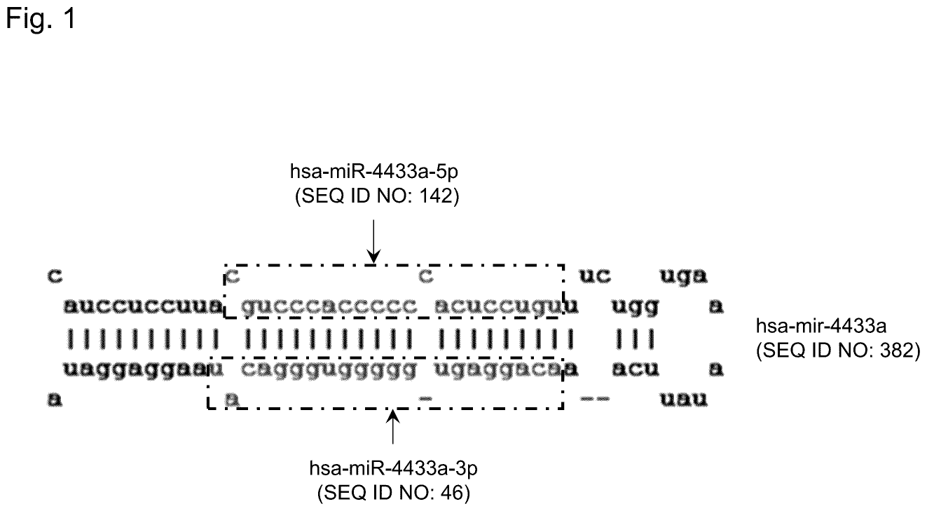

In this context, the sequences of the nucleic acid probes and the primers for detecting the polynucleotide consisting of a nucleotide sequence represented by any of SEQ ID NOs: 1 to 329 do not exist as miRNAs or precursors thereof in the living body or in vivo. For example, the nucleotide sequences represented by SEQ ID NO: 142 and SEQ ID NO: 46 are produced from the precursor represented by SEQ ID NO: 382. This precursor has a hairpin-like structure as shown in FIG. 1, and the nucleotide sequences represented by SEQ ID NO: 142 and SEQ ID NO: 46 have mismatch sequences with each other. As such, a nucleotide sequence completely complementary to the nucleotide sequence represented by SEQ ID NO: 142 or SEQ ID NO: 46 does not naturally occur in vivo. Therefore, the nucleic acid probes and the primers for detecting the nucleotide sequence represented by any of SEQ ID NOs: 1 to 329 can have artificial nucleotide sequences that do not exist in the living body or in vivo.

### 3. Kit or Device for Detection of Lung Cancer

The present invention also provides a kit or a device for detecting lung cancer, comprising one or more polynucleotides (which may include a variant, a fragment, or a derivative thereof) that can be used as nucleic acid probes or primers in the present invention for measuring target nucleic acids as lung cancer markers.

The target nucleic acids as lung cancer markers according to the present invention are preferably selected from the following group A:

### Group A:

miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p.

Additional target nucleic acids that may be optionally used in the measurement are preferably selected from the following group B:

### Group B:

miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p.

The kit or the device of the present invention comprises one or more nucleic acids capable of specifically binding to any of the target nucleic acids as the lung cancer markers described above or nucleic acids for detecting the target nucleic acids, preferably one or more polynucleotides selected from the polynucleotides described in the preceding Section 2, or variants thereof.

Specifically, the kit or the device of the present invention can comprise at least one polynucleotide comprising (or consisting of), for example, a nucleotide sequence represented by any of SEQ ID NOs: 1 to 163, or a nucleotide sequence derived from the nucleotide sequence in which the nucleic acid u is replaced with t, polynucleotide(s) comprising (or consisting of) a complementary sequence thereof, a polynucleotide(s) hybridizing under stringent conditions to any of these polynucleotides, or a variant(s) or a fragment(s) comprising 15 or more consecutive nucleotides of any of these polynucleotide sequences.

The kit or the device of the present invention can further comprise one or more polynucleotides comprising (or consisting of), for example, a nucleotide sequence represented by any of SEQ ID NOs: 164 to 329, or a nucleotide sequence derived from the nucleotide sequence in which the nucleic acid u is replaced with t, a polynucleotide(s) comprising (or consisting of) a complementary sequence thereof, a polynucleotide(s) hybridizing under stringent conditions to any of these polynucleotides, a variant(s) or a fragment(s) comprising 15 or more consecutive nucleotides of any of these polynucleotide sequences.

The fragment or fragments that can be comprised in the kit or the device of the present invention is/are, for example, one or more polynucleotides, preferably two or more polynucleotides, selected from the group consisting of the following polynucleotides (1) and (2):
(1) a polynucleotide comprising 15 or more consecutive nucleotides derived from a nucleotide sequence represented by any of SEQ ID NOs: 1 to 163 in which the nucleic acid u is replaced with t, or a complementary sequence thereof; and
(2) a polynucleotide comprising 15 or more consecutive nucleotides derived from a nucleotide sequence represented by any of SEQ ID NOs: 164 to 329 in which the nucleic acid u is replaced with t, or a complementary sequence thereof.

In a preferred embodiment, the polynucleotide is a polynucleotide consisting of a nucleotide sequence represented by any of SEQ ID NOs: 1 to 163, or a nucleotide sequence derived from the nucleotide sequence in which the nucleic acid u is replaced with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 164 to 329, or a nucleotide sequence derived from the nucleotide sequence in which the nucleic acid u is replaced with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the fragment can be a polynucleotide comprising 15 or more, preferably 17 or more, more preferably 19 or more consecutive nucleotides.

In the present invention, the size of the polynucleotide fragment is the number of nucleotides in the range from, for example, 15 consecutive nucleotides to less than the total number of nucleotides of the sequence, from 17 consecutive nucleotides to less than the total number of nucleotides of the sequence, or from 19 consecutive nucleotides to less than the total number of nucleotides of the sequence, in the nucleotide sequence of each polynucleotide.

Examples of the combination of the above-mentioned polynucleotides as target nucleic acids in the kit or the device of the present invention can include a single (one) polynucleotide or combinations of 2, 3, 4, 5, 6, 7, 8, 9, 10 or more of the above-mentioned polynucleotides consisting of the nucleotide sequences represented by SEQ ID NOs: 1 to 329 as shown in Table 1 above. However, these are given merely for illustrative purposes, and all of various other possible combinations are included in the present invention.

Examples of the combinations of target nucleic acids in the kit or the device for discriminating lung cancer patients from test subjects without lung cancer, such as healthy subjects, benign bone and soft tissue tumor patients and benign breast disease patients, or patients having a cancer other than lung cancer, according to the present invention can include combinations of two or more of the above-mentioned polynucleotides consisting of the nucleotide sequences represented by SEQ ID NOs as shown in Table 1. For example, any two or more of the above-mentioned polynucleotides consisting of nucleotide sequences represented by SEQ ID NOs: 1 to 329 can be combined. Among them, at least one polynucleotide of the newly found polynucleotides consisting of the nucleotide sequences represented by SEQ ID NOs: 1 to 163 is preferably selected. Among them, particularly, a combination comprising at least one polynucleotide selected from the group consisting of the polynucleotides of SEQ ID NOs: 18, 4, 130, 2, 9, 17, and 121 (hereinafter, this group is referred to as “cancer type-specific polynucleotide group”) is more preferred.

Non-limiting examples of the combination comprising the polynucleotide consisting of the nucleotide sequence represented by SEQ ID NO: 18 or a complementary sequence thereof are listed below as combinations of target nucleic acids:
(1) a combination of SEQ ID NOs: 18, and 164;
(2) a combination of SEQ ID NOs: 18, 164, and 255;
(3) a combination of SEQ ID NOs: 18, 164, and 300;
(4) a combination of SEQ ID NOs: 18, 164, and 190;
(5) a combination of SEQ ID NOs: 18, 85, and 164;
(6) a combination of SEQ ID NOs: 18, 147, and 164;
(7) a combination of SEQ ID NOs: 18, 22, and 164;
(8) a combination of SEQ ID NOs: 18, 164, and 312;
(9) a combination of SEQ ID NOs: 18, 66, and 164;
(10) a combination of SEQ ID NOs: 18, 78, and 164;
(11) a combination of SEQ ID NOs: 18, 27, and 164;
(12) a combination of SEQ ID NOs: 18, 164, and 207;
(13) a combination of SEQ ID NOs: 18, 82, and 164;
(14) a combination of SEQ ID NOs: 18, 164, and 263;
(15) a combination of SEQ ID NOs: 18, 164, and 168;
(16) a combination of SEQ ID NOs: 18, 34, and 164;
(17) a combination of SEQ ID NOs: 18, 39, and 164;
(18) a combination of SEQ ID NOs: 18, 57, and 164;
(19) a combination of SEQ ID NOs: 18, 121, and 164;
(20) a combination of SEQ ID NOs: 18, 107, and 164;
(21) a combination of SEQ ID NOs: 18, 70, and 164;
(22) a combination of SEQ ID NOs: 18, 50, and 164;
(23) a combination of SEQ ID NOs: 18, 164, and 250;
(24) a combination of SEQ ID NOs: 18, 164, and 315;
(25) a combination of SEQ ID NOs: 18, 164, and 211;
(26) a combination of SEQ ID NOs: 18, 164, and 326;
(27) a combination of SEQ ID NOs: 18, 164, and 308;
(28) a combination of SEQ ID NOs: 18, 164, and 268;
(29) a combination of SEQ ID NOs: 18, 164, and 191;
(30) a combination of SEQ ID NOs: 18, 149, and 165;
(31) a combination of SEQ ID NOs: 18, 121, 130, and 164;
(32) a combination of SEQ ID NOs: 18, 164, 255, and 316;
(33) a combination of SEQ ID NOs: 18, 121, 164, and 255;
(34) a combination of SEQ ID NOs: 18, 147, 164, and 255;
(35) a combination of SEQ ID NOs: 18, 27, 164, and 255;
(36) a combination of SEQ ID NOs: 18, 34, 164, and 255;
(37) a combination of SEQ ID NOs: 18, 47, 164, and 255;
(38) a combination of SEQ ID NOs: 18, 158, 164, and 255;
(39) a combination of SEQ ID NOs: 18, 164, 220, and 255;
(40) a combination of SEQ ID NOs: 18, 88, 164, and 255;
(41) a combination of SEQ ID NOs: 18, 130, 164, and 268;
(42) a combination of SEQ ID NOs: 18, 164, 255, and 321;
(43) a combination of SEQ ID NOs: 18, 164, 184, and 255;
(44) a combination of SEQ ID NOs: 18, 152, 164, and 255;
(45) a combination of SEQ ID NOs: 18, 164, 185, and 255;
(46) a combination of SEQ ID NOs: 18, 164, 238, and 255;
(47) a combination of SEQ ID NOs: 18, 164, 255, and 256;
(48) a combination of SEQ ID NOs: 18, 127, 164, and 255;
(49) a combination of SEQ ID NOs: 18, 164, 222, and 255;
(50) a combination of SEQ ID NOs: 18, 139, 164, and 255;
(51) a combination of SEQ ID NOs: 18, 39, 164, and 255;
(52) a combination of SEQ ID NOs: 18, 164, 255, and 295;
(53) a combination of SEQ ID NOs: 18, 146, 164, and 255;
(54) a combination of SEQ ID NOs: 18, 164, 211, and 255;
(55) a combination of SEQ ID NOs: 18, 164, 255, and 322;
(56) a combination of SEQ ID NOs: 18, 164, 255, and 318;
(57) a combination of SEQ ID NOs: 18, 121, 164, and 201;
(58) a combination of SEQ ID NOs: 18, 147, 164, and 300;
(59) a combination of SEQ ID NOs: 18, 121, 151, and 164;
(60) a combination of SEQ ID NOs: 18, 164, 211, and 300;
(61) a combination of SEQ ID NOs: 18, 95, 164, and 268;
(62) a combination of SEQ ID NOs: 18, 164, 231, and 268;
(63) a combination of SEQ ID NOs: 18, 147, 164, and 268;
(64) a combination of SEQ ID NOs: 18, 164, 188, and 268;
(65) a combination of SEQ ID NOs: 18, 164, 268, and 312;
(66) a combination of SEQ ID NOs: 18, 39, 164, and 300;
(67) a combination of SEQ ID NOs: 18, 95, 121, and 164;
(68) a combination of SEQ ID NOs: 18, 93, 164, and 268;
(69) a combination of SEQ ID NOs: 18, 164, 268, and 308;
(70) a combination of SEQ ID NOs: 18, 107, 121, and 164;
(71) a combination of SEQ ID NOs: 18, 164, 218, and 268;
(72) a combination of SEQ ID NOs: 18, 164, 202, and 268;
(73) a combination of SEQ ID NOs: 13, 18, 130, and 165;
(74) a combination of SEQ ID NOs: 18, 149, 165, and 168;
(75) a combination of SEQ ID NOs: 18, 164, 242, and 268;
(76) a combination of SEQ ID NOs: 18, 164, 214, and 268;
(77) a combination of SEQ ID NOs: 18, 164, 268, and 313;
(78) a combination of SEQ ID NOs: 18, 162, 164, and 268;
(79) a combination of SEQ ID NOs: 18, 150, 164, and 268;
(80) a combination of SEQ ID NOs: 18, 164, 268, and 315;
(81) a combination of SEQ ID NOs: 18, 152, 164, and 268;
(82) a combination of SEQ ID NOs: 18, 164, 268, and 325;
(83) a combination of SEQ ID NOs: 18, 121, 149, and 165;
(84) a combination of SEQ ID NOs: 13, 18, 165, and 260;
(85) a combination of SEQ ID NOs: 13, 18, 165, and 268;
(86) a combination of SEQ ID NOs: 13, 18, 121, and 165;
(87) a combination of SEQ ID NOs: 13, 18, 165, and 168;
(88) a combination of SEQ ID NOs: 18, 149, 165, and 268;
(89) a combination of SEQ ID NOs: 13, 18, 83, and 165;
(90) a combination of SEQ ID NOs: 13, 18, 165, and 263;
(91) a combination of SEQ ID NOs: 2, 18, 165, and 268;
(92) a combination of SEQ ID NOs: 13, 18, 165, and 211;
(93) a combination of SEQ ID NOs: 13, 18, 165, and 256;
(94) a combination of SEQ ID NOs: 13, 18, 165, and 276;
(95) a combination of SEQ ID NOs: 13, 18, 165, and 302;
(96) a combination of SEQ ID NOs: 13, 18, 165, and 190;
(97) a combination of SEQ ID NOs: 18, 121, 130, 136, and 164;
(98) a combination of SEQ ID NOs: 18, 121, 130, 164, and 314;
(99) a combination of SEQ ID NOs: 18, 114, 121, 130, and 164;
(100) a combination of SEQ ID NOs: 18, 121, 130, 164, and 214;
(101) a combination of SEQ ID NOs: 18, 121, 130, 164, and 193;
(102) a combination of SEQ ID NOs: 18, 130, 164, 255, and 268;
(103) a combination of SEQ ID NOs: 18, 121, 130, 164, and 320;
(104) a combination of SEQ ID NOs: 18, 121, 130, 164, and 301;
(105) a combination of SEQ ID NOs: 18, 121, 130, 144, and 164;
(106) a combination of SEQ ID NOs: 18, 121, 130, 164, and 168;
(107) a combination of SEQ ID NOs: 18, 121, 130, 164, and 205;
(108) a combination of SEQ ID NOs: 18, 121, 130, 158, and 164;
(109) a combination of SEQ ID NOs: 18, 121, 130, 164, and 260;
(110) a combination of SEQ ID NOs: 18, 106, 121, 130, and 164;
(111) a combination of SEQ ID NOs: 18, 121, 130, 164, and 318;
(112) a combination of SEQ ID NOs: 18, 121, 130, 164, and 286;
(113) a combination of SEQ ID NOs: 18, 121, 130, 164, and 315;
(114) a combination of SEQ ID NOs: 18, 121, 130, 164, and 237;
(115) a combination of SEQ ID NOs: 18, 121, 130, 164, and 184;
(116) a combination of SEQ ID NOs: 18, 121, 130, 164, and 270;
(117) a combination of SEQ ID NOs: 18, 121, 130, 164, and 309;
(118) a combination of SEQ ID NOs: 18, 121, 130, 164, and 278;
(119) a combination of SEQ ID NOs: 18, 82, 121, 130, and 164;
(120) a combination of SEQ ID NOs: 18, 23, 121, 130, and 164;
(121) a combination of SEQ ID NOs: 18, 121, 130, 164, and 189;
(122) a combination of SEQ ID NOs: 18, 121, 130, 152, and 164;
(123) a combination of SEQ ID NOs: 18, 121, 130, 164, and 213;
(124) a combination of SEQ ID NOs: 18, 121, 130, 164, and 229;
(125) a combination of SEQ ID NOs: 18, 57, 121, 130, and 164;
(126) a combination of SEQ ID NOs: 18, 121, 130, 142, and 164;
(127) a combination of SEQ ID NOs: 18, 121, 130, 155, and 164;
(128) a combination of SEQ ID NOs: 18, 39, 121, 130, and 164;
(129) a combination of SEQ ID NOs: 18, 27, 130, 164, and 268;
(130) a combination of SEQ ID NOs: 18, 33, 121, 130, and 164;
(131) a combination of SEQ ID NOs: 18, 121, 126, 130, and 164;
(132) a combination of SEQ ID NOs: 18, 121, 130, 164, and 319;
(133) a combination of SEQ ID NOs: 18, 22, 121, 130, and 164;
(134) a combination of SEQ ID NOs: 18, 59, 121, 130, and 164;
(135) a combination of SEQ ID NOs: 18, 27, 121, 130, and 164;
(136) a combination of SEQ ID NOs: 18, 130, 164, 268, and 317;
(137) a combination of SEQ ID NOs: 18, 121, 130, 164, and 201;
(138) a combination of SEQ ID NOs: 18, 34, 164, 211, and 255;
(139) a combination of SEQ ID NOs: 18, 19, 121, 130, and 164;
(140) a combination of SEQ ID NOs: 18, 74, 130, 164, and 268;
(141) a combination of SEQ ID NOs: 18, 130, 164, 264, and 268;
(142) a combination of SEQ ID NOs: 18, 39, 164, 255, and 328;
(143) a combination of SEQ ID NOs: 18, 39, 164, 226, and 255;
(144) a combination of SEQ ID NOs: 18, 95, 121, 164, and 188;
(145) a combination of SEQ ID NOs: 13, 18, 121, 130, and 165;
(146) a combination of SEQ ID NOs: 13, 18, 130, 165, and 268;
(147) a combination of SEQ ID NOs: 18, 151, 164, 268, and 315;
(148) a combination of SEQ ID NOs: 18, 147, 164, 184, and 268;
(149) a combination of SEQ ID NOs: 18, 149, 165, 168, and 268;
(150) a combination of SEQ ID NOs: 13, 18, 165, 268, and 276;
(151) a combination of SEQ ID NOs: 2, 18, 165, 268, and 301;
(152) a combination of SEQ ID NOs: 2, 18, 165, 268, and 315;
(153) a combination of SEQ ID NOs: 13, 18, 165, 183, and 268; and
(154) a combination of SEQ ID NOs: 13, 18, 165, 184, and 268.

Non-limiting examples of the combination comprising the polynucleotide consisting of the nucleotide sequence represented by SEQ ID NO: 4 or a complementary sequence thereof are further listed below as combinations of target nucleic acids:
(1) a combination of SEQ ID NOs: 4, and 164;
(2) a combination of SEQ ID NOs: 4, 165, and 168;
(3) a combination of SEQ ID NOs: 4, 165, 168, and 246;
(4) a combination of SEQ ID NOs: 4, 128, 165, and 168;
(5) a combination of SEQ ID NOs: 4, 117, 165, and 168;
(6) a combination of SEQ ID NOs: 4, 159, 165, and 168;
(7) a combination of SEQ ID NOs: 4, 165, 168, and 260;
(8) a combination of SEQ ID NOs: 4, 17, 165, and 168;
(9) a combination of SEQ ID NOs: 4, 165, 168, and 173;
(10) a combination of SEQ ID NOs: 4, 80, 165, and 168;
(11) a combination of SEQ ID NOs: 4, 99, 165, and 168;
(12) a combination of SEQ ID NOs: 2, 4, 168, and 246;
(13) a combination of SEQ ID NOs: 4, 17, 115, and 168;
(14) a combination of SEQ ID NOs: 4, 17, 115, and 302;
(15) a combination of SEQ ID NOs: 4, 94, 173, and 183;
(16) a combination of SEQ ID NOs: 2, 4, 173, and 183;
(17) a combination of SEQ ID NOs: 2, 4, 115, and 168;
(18) a combination of SEQ ID NOs: 4, 17, 115, and 184;
(19) a combination of SEQ ID NOs: 4, 17, 165, 168, and 173;
(20) a combination of SEQ ID NOs: 4, 17, 165, 168, and 223;
(21) a combination of SEQ ID NOs: 4, 128, 129, 165, and 168;
(22) a combination of SEQ ID NOs: 2, 4, 130, 168, and 246;
(23) a combination of SEQ ID NOs: 4, 17, 128, 165, and 168;
(24) a combination of SEQ ID NOs: 4, 17, 165, 168, and 169;
(25) a combination of SEQ ID NOs: 4, 17, 117, 165, and 168;
(26) a combination of SEQ ID NOs: 4, 17, 165, 168, and 323;
(27) a combination of SEQ ID NOs: 4, 17, 81, 165, and 168;
(28) a combination of SEQ ID NOs: 4, 17, 165, 168, and 253;
(29) a combination of SEQ ID NOs: 4, 17, 162, 165, and 168;
(30) a combination of SEQ ID NOs: 2, 4, 168, 201, and 246;
(31) a combination of SEQ ID NOs: 4, 17, 141, 165, and 168;
(32) a combination of SEQ ID NOs: 4, 17, 129, 165, and 168;
(33) a combination of SEQ ID NOs: 4, 17, 165, 168, and 258;
(34) a combination of SEQ ID NOs: 4, 17, 165, 168, and 190;
(35) a combination of SEQ ID NOs: 4, 17, 115, 168, and 177;
(36) a combination of SEQ ID NOs: 4, 17, 165, 168, and 191;
(37) a combination of SEQ ID NOs: 4, 17, 158, 165, and 168;
(38) a combination of SEQ ID NOs: 4, 17, 165, 168, and 184;
(39) a combination of SEQ ID NOs: 4, 17, 94, 165, and 168;
(40) a combination of SEQ ID NOs: 4, 17, 165, 168, and 296;
(41) a combination of SEQ ID NOs: 4, 17, 165, 168, and 307;
(42) a combination of SEQ ID NOs: 4, 17, 123, 165, and 168;
(43) a combination of SEQ ID NOs: 4, 17, 39, 165, and 168;
(44) a combination of SEQ ID NOs: 4, 17, 145, 165, and 168;
(45) a combination of SEQ ID NOs: 4, 17, 165, 168, and 286;
(46) a combination of SEQ ID NOs: 4, 17, 73, 165, and 168;
(47) a combination of SEQ ID NOs: 4, 17, 115, 165, and 168;
(48) a combination of SEQ ID NOs: 4, 17, 108, 165, and 168;
(49) a combination of SEQ ID NOs: 4, 17, 156, 165, and 168;
(50) a combination of SEQ ID NOs: 4, 17, 165, 168, and 249;
(51) a combination of SEQ ID NOs: 4, 17, 131, 165, and 168;
(52) a combination of SEQ ID NOs: 4, 17, 165, 168, and 304;
(53) a combination of SEQ ID NOs: 4, 17, 157, 165, and 168;
(54) a combination of SEQ ID NOs: 4, 17, 165, 168, and 318;
(55) a combination of SEQ ID NOs: 4, 17, 74, 165, and 168;
(56) a combination of SEQ ID NOs: 4, 17, 165, 168, and 216;
(57) a combination of SEQ ID NOs: 4, 17, 165, 168, and 309;
(58) a combination of SEQ ID NOs: 4, 17, 165, 168, and 236;
(59) a combination of SEQ ID NOs: 4, 17, 165, 168, and 324;
(60) a combination of SEQ ID NOs: 2, 4, 111, 168, and 173;
(61) a combination of SEQ ID NOs: 4, 17, 115, 130, and 168;
(62) a combination of SEQ ID NOs: 2, 4, 130, 168, and 173;
(63) a combination of SEQ ID NOs: 4, 17, 111, 115, and 168;
(64) a combination of SEQ ID NOs: 2, 4, 168, 173, and 201;
(65) a combination of SEQ ID NOs: 4, 17, 115, 160, and 168;
(66) a combination of SEQ ID NOs: 4, 17, 115, 168, and 246;
(67) a combination of SEQ ID NOs: 2, 4, 115, 168, and 173;
(68) a combination of SEQ ID NOs: 4, 17, 115, 168, and 201;
(69) a combination of SEQ ID NOs: 4, 17, 115, 168, and 217;
(70) a combination of SEQ ID NOs: 2, 4, 17, 115, and 168;
(71) a combination of SEQ ID NOs: 4, 17, 115, 140, and 168; and
(72) a combination of SEQ ID NOs: 4, 17, 102, 115, and 168.

Non-limiting examples of the combination comprising the polynucleotide consisting of the nucleotide sequence represented by SEQ ID NO: 130 or a complementary sequence thereof are further listed below as combinations of target nucleic acids:
(1) a combination of SEQ ID NOs: 121, 130, and 164;
(2) a combination of SEQ ID NOs: 18, 121, 130, and 164;
(3) a combination of SEQ ID NOs: 18, 130, 164, and 268;
(4) a combination of SEQ ID NOs: 13, 18, 130, and 165;
(5) a combination of SEQ ID NOs: 18, 121, 130, 136, and 164;
(6) a combination of SEQ ID NOs: 18, 121, 130, 164, and 314;
(7) a combination of SEQ ID NOs: 18, 114, 121, 130, and 164;
(8) a combination of SEQ ID NOs: 18, 121, 130, 164, and 214;
(9) a combination of SEQ ID NOs: 18, 121, 130, 164, and 193;
(10) a combination of SEQ ID NOs: 18, 130, 164, 255, and 268;
(11) a combination of SEQ ID NOs: 18, 121, 130, 164, and 320;
(12) a combination of SEQ ID NOs: 18, 121, 130, 164, and 301;
(13) a combination of SEQ ID NOs: 18, 121, 130, 144, and 164;
(14) a combination of SEQ ID NOs: 18, 121, 130, 164, and 168;
(15) a combination of SEQ ID NOs: 18, 121, 130, 164, and 205;
(16) a combination of SEQ ID NOs: 18, 121, 130, 158, and 164;
(17) a combination of SEQ ID NOs: 18, 121, 130, 164, and 260;
(18) a combination of SEQ ID NOs: 18, 106, 121, 130, and 164;
(19) a combination of SEQ ID NOs: 18, 121, 130, 164, and 318;
(20) a combination of SEQ ID NOs: 18, 121, 130, 164, and 286;
(21) a combination of SEQ ID NOs: 18, 121, 130, 164, and 315;
(22) a combination of SEQ ID NOs: 18, 121, 130, 164, and 237;
(23) a combination of SEQ ID NOs: 18, 121, 130, 164, and 184;
(24) a combination of SEQ ID NOs: 18, 121, 130, 164, and 270;
(25) a combination of SEQ ID NOs: 18, 121, 130, 164, and 309;
(26) a combination of SEQ ID NOs: 18, 121, 130, 164, and 278;
(27) a combination of SEQ ID NOs: 18, 82, 121, 130, and 164;
(28) a combination of SEQ ID NOs: 18, 23, 121, 130, and 164;
(29) a combination of SEQ ID NOs: 18, 121, 130, 164, and 189;
(30) a combination of SEQ ID NOs: 18, 121, 130, 152, and 164;
(31) a combination of SEQ ID NOs: 18, 121, 130, 164, and 213;
(32) a combination of SEQ ID NOs: 18, 121, 130, 164, and 229;
(33) a combination of SEQ ID NOs: 18, 57, 121, 130, and 164;
(34) a combination of SEQ ID NOs: 18, 121, 130, 142, and 164;
(35) a combination of SEQ ID NOs: 18, 121, 130, 155, and 164;
(36) a combination of SEQ ID NOs: 18, 39, 121, 130, and 164;
(37) a combination of SEQ ID NOs: 18, 27, 130, 164, and 268;
(38) a combination of SEQ ID NOs: 18, 33, 121, 130, and 164;
(39) a combination of SEQ ID NOs: 18, 121, 126, 130, and 164;
(40) a combination of SEQ ID NOs: 18, 121, 130, 164, and 319;
(41) a combination of SEQ ID NOs: 18, 22, 121, 130, and 164;
(42) a combination of SEQ ID NOs: 18, 59, 121, 130, and 164;
(43) a combination of SEQ ID NOs: 18, 27, 121, 130, and 164;
(44) a combination of SEQ ID NOs: 18, 130, 164, 268, and 317;
(45) a combination of SEQ ID NOs: 18, 121, 130, 164, and 201;
(46) a combination of SEQ ID NOs: 18, 19, 121, 130, and 164;
(47) a combination of SEQ ID NOs: 18, 74, 130, 164, and 268;
(48) a combination of SEQ ID NOs: 18, 130, 164, 264, and 268;
(49) a combination of SEQ ID NOs: 2, 4, 130, 168, and 246;
(50) a combination of SEQ ID NOs: 2, 9, 130, 168, and 246;
(51) a combination of SEQ ID NOs: 13, 18, 121, 130, and 165;
(52) a combination of SEQ ID NOs: 13, 18, 130, 165, and 268;
(53) a combination of SEQ ID NOs: 4, 17, 115, 130, and 168;
(54) a combination of SEQ ID NOs: 2, 4, 130, 168, and 173;
(55) a combination of SEQ ID NOs: 2, 9, 130, 168, and 173;
(56) a combination of SEQ ID NOs: 2, 111, 130, 168, and 173;
(57) a combination of SEQ ID NOs: 2, 83, 130, 168, and 173;
(58) a combination of SEQ ID NOs: 2, 6, 130, 168, and 173;
(59) a combination of SEQ ID NOs: 2, 6, 130, 173, and 184;
(60) a combination of SEQ ID NOs: 2, 130, 168, 173, and 213;
(61) a combination of SEQ ID NOs: 2, 5, 130, 168, and 173; and
(62) a combination of SEQ ID NOs: 2, 130, 168, 173, and 249.

Non-limiting examples of the combination comprising the polynucleotide consisting of the nucleotide sequence represented by SEQ ID NO: 2 or a complementary sequence thereof are further listed below as combinations of target nucleic acids:
(1) a combination of SEQ ID NOs: 2, 121, 165, and 168;
(2) a combination of SEQ ID NOs: 2, 165, 168, and 268;
(3) a combination of SEQ ID NOs: 2, 4, 168, and 246;
(4) a combination of SEQ ID NOs: 2, 18, 165, and 268;
(5) a combination of SEQ ID NOs: 2, 4, 173, and 183;
(6) a combination of SEQ ID NOs: 2, 4, 115, and 168;
(7) a combination of SEQ ID NOs: 2, 9, 168, and 246;
(8) a combination of SEQ ID NOs: 2, 111, 168, and 246;
(9) a combination of SEQ ID NOs: 2, 111, 168, and 173;
(10) a combination of SEQ ID NOs: 2, 102, 168, and 246;
(11) a combination of SEQ ID NOs: 2, 4, 130, 168, and 246;
(12) a combination of SEQ ID NOs: 2, 4, 168, 201, and 246;
(13) a combination of SEQ ID NOs: 2, 9, 130, 168, and 246;
(14) a combination of SEQ ID NOs: 2, 4, 111, 168, and 173;
(15) a combination of SEQ ID NOs: 2, 4, 130, 168, and 173;
(16) a combination of SEQ ID NOs: 2, 4, 168, 173, and 201;
(17) a combination of SEQ ID NOs: 2, 4, 115, 168, and 173;
(18) a combination of SEQ ID NOs: 2, 9, 130, 168, and 173;
(19) a combination of SEQ ID NOs: 2, 4, 17, 115, and 168;
(20) a combination of SEQ ID NOs: 2, 111, 168, 173, and 268;
(21) a combination of SEQ ID NOs: 2, 18, 165, 268, and 301;
(22) a combination of SEQ ID NOs: 2, 18, 165, 268, and 315;
(23) a combination of SEQ ID NOs: 2, 111, 130, 168, and 173;
(24) a combination of SEQ ID NOs: 2, 83, 130, 168, and 173;
(25) a combination of SEQ ID NOs: 2, 6, 130, 168, and 173;
(26) a combination of SEQ ID NOs: 2, 111, 168, 173, and 223;
(27) a combination of SEQ ID NOs: 2, 5, 111, 168, and 173;
(28) a combination of SEQ ID NOs: 2, 6, 130, 173, and 184;
(29) a combination of SEQ ID NOs: 2, 39, 111, 168, and 173;
(30) a combination of SEQ ID NOs: 2, 111, 168, 173, and 222;
(31) a combination of SEQ ID NOs: 2, 111, 152, 168, and 173;
(32) a combination of SEQ ID NOs: 2, 111, 168, 173, and 241;
(33) a combination of SEQ ID NOs: 2, 130, 168, 173, and 213;
(34) a combination of SEQ ID NOs: 2, 111, 168, 173, and 184;
(35) a combination of SEQ ID NOs: 2, 102, 111, 168, and 173;
(36) a combination of SEQ ID NOs: 2, 5, 130, 168, and 173;
(37) a combination of SEQ ID NOs: 2, 111, 168, 173, and 234;
(38) a combination of SEQ ID NOs: 2, 111, 168, 173, and 230;
(39) a combination of SEQ ID NOs: 2, 111, 168, 173, and 307;
(40) a combination of SEQ ID NOs: 2, 130, 168, 173, and 249;
(41) a combination of SEQ ID NOs: 2, 111, 158, 168, and 173; and
(42) a combination of SEQ ID NOs: 2, 39, 168, 169, and 173.

Non-limiting examples of the combination comprising the polynucleotide consisting of the nucleotide sequence represented by SEQ ID NO: 9 or a complementary sequence thereof are further listed below as combinations of target nucleic acids:
(1) a combination of SEQ ID NOs: 9, 165, and 168;
(2) a combination of SEQ ID NOs: 9, 165, 168, and 173;
(3) a combination of SEQ ID NOs: 9, 128, 165, and 168;
(4) a combination of SEQ ID NOs: 9, 17, 165, and 168;
(5) a combination of SEQ ID NOs: 9, 80, 165, and 168;
(6) a combination of SEQ ID NOs: 2, 9, 168, and 246;
(7) a combination of SEQ ID NOs: 5, 9, 165, 168, and 173;
(8) a combination of SEQ ID NOs: 9, 128, 129, 165, and 168;
(9) a combination of SEQ ID NOs: 2, 9, 130, 168, and 246;
(10) a combination of SEQ ID NOs: 9, 17, 159, 165, and 168;
(11) a combination of SEQ ID NOs: 9, 17, 165, 168, and 173; and
(12) a combination of SEQ ID NOs: 2, 9, 130, 168, and 173.

Non-limiting examples of the combination comprising the polynucleotide consisting of the nucleotide sequence represented by SEQ ID NO: 17 or a complementary sequence thereof are further listed below as combinations of target nucleic acids:
(1) a combination of SEQ ID NOs: 17, 164, and 168;
(2) a combination of SEQ ID NOs: 4, 17, 165, and 168;
(3) a combination of SEQ ID NOs: 9, 17, 165, and 168;
(4) a combination of SEQ ID NOs: 4, 17, 115, and 168;
(5) a combination of SEQ ID NOs: 4, 17, 115, and 302;
(6) a combination of SEQ ID NOs: 4, 17, 115, and 184;
(7) a combination of SEQ ID NOs: 4, 17, 165, 168, and 173;
(8) a combination of SEQ ID NOs: 4, 17, 165, 168, and 223;
(9) a combination of SEQ ID NOs: 4, 17, 128, 165, and 168;
(10) a combination of SEQ ID NOs: 4, 17, 165, 168, and 169;
(11) a combination of SEQ ID NOs: 4, 17, 117, 165, and 168;
(12) a combination of SEQ ID NOs: 4, 17, 165, 168, and 323;
(13) a combination of SEQ ID NOs: 4, 17, 81, 165, and 168;
(14) a combination of SEQ ID NOs: 4, 17, 165, 168, and 253;
(15) a combination of SEQ ID NOs: 4, 17, 162, 165, and 168;
(16) a combination of SEQ ID NOs: 4, 17, 141, 165, and 168;
(17) a combination of SEQ ID NOs: 4, 17, 129, 165, and 168;
(18) a combination of SEQ ID NOs: 4, 17, 165, 168, and 258;
(19) a combination of SEQ ID NOs: 4, 17, 165, 168, and 190;
(20) a combination of SEQ ID NOs: 4, 17, 115, 168, and 177;
(21) a combination of SEQ ID NOs: 4, 17, 165, 168, and 191;
(22) a combination of SEQ ID NOs: 4, 17, 158, 165, and 168;
(23) a combination of SEQ ID NOs: 4, 17, 165, 168, and 184;
(24) a combination of SEQ ID NOs: 4, 17, 94, 165, and 168;
(25) a combination of SEQ ID NOs: 4, 17, 165, 168, and 296;
(26) a combination of SEQ ID NOs: 4, 17, 165, 168, and 307;
(27) a combination of SEQ ID NOs: 4, 17, 123, 165, and 168;
(28) a combination of SEQ ID NOs: 4, 17, 39, 165, and 168;
(29) a combination of SEQ ID NOs: 4, 17, 145, 165, and 168;
(30) a combination of SEQ ID NOs: 4, 17, 165, 168, and 286;
(31) a combination of SEQ ID NOs: 4, 17, 73, 165, and 168;
(32) a combination of SEQ ID NOs: 4, 17, 115, 165, and 168;
(33) a combination of SEQ ID NOs: 4, 17, 108, 165, and 168;
(34) a combination of SEQ ID NOs: 4, 17, 156, 165, and 168;
(35) a combination of SEQ ID NOs: 4, 17, 165, 168, and 249;
(36) a combination of SEQ ID NOs: 4, 17, 131, 165, and 168;
(37) a combination of SEQ ID NOs: 4, 17, 165, 168, and 304;
(38) a combination of SEQ ID NOs: 4, 17, 157, 165, and 168;
(39) a combination of SEQ ID NOs: 4, 17, 165, 168, and 318;
(40) a combination of SEQ ID NOs: 4, 17, 74, 165, and 168;
(41) a combination of SEQ ID NOs: 4, 17, 165, 168, and 216;
(42) a combination of SEQ ID NOs: 4, 17, 165, 168, and 309;
(43) a combination of SEQ ID NOs: 4, 17, 165, 168, and 236;
(44) a combination of SEQ ID NOs: 4, 17, 165, 168, and 324;
(45) a combination of SEQ ID NOs: 9, 17, 159, 165, and 168;
(46) a combination of SEQ ID NOs: 4, 17, 115, 130, and 168;
(47) a combination of SEQ ID NOs: 4, 17, 111, 115, and 168;
(48) a combination of SEQ ID NOs: 9, 17, 165, 168, and 173;
(49) a combination of SEQ ID NOs: 4, 17, 115, 160, and 168;
(50) a combination of SEQ ID NOs: 4, 17, 115, 168, and 246;
(51) a combination of SEQ ID NOs: 4, 17, 115, 168, and 201;
(52) a combination of SEQ ID NOs: 4, 17, 115, 168, and 217;
(53) a combination of SEQ ID NOs: 2, 4, 17, 115, and 168;
(54) a combination of SEQ ID NOs: 4, 17, 115, 140, and 168; and
(55) a combination of SEQ ID NOs: 4, 17, 102, 115, and 168.

Non-limiting examples of the combination comprising the polynucleotide consisting of the nucleotide sequence represented by SEQ ID NO: 121 or a complementary sequence thereof are further listed below as combinations of target nucleic acids:
(1) a combination of SEQ ID NOs: 121, 130, and 164;
(2) a combination of SEQ ID NOs: 18, 121, and 164;
(3) a combination of SEQ ID NOs: 121, 164, and 168;
(4) a combination of SEQ ID NOs: 121, 164, and 328;
(5) a combination of SEQ ID NOs: 121, 164, and 211;
(6) a combination of SEQ ID NOs: 95, 121, and 164;
(7) a combination of SEQ ID NOs: 6, 121, and 165;
(8) a combination of SEQ ID NOs: 18, 121, 130, and 164;
(9) a combination of SEQ ID NOs: 18, 121, 164, and 255;
(10) a combination of SEQ ID NOs: 18, 121, 164, and 201;
(11) a combination of SEQ ID NOs: 18, 121, 151, and 164;
(12) a combination of SEQ ID NOs: 18, 95, 121, and 164;
(13) a combination of SEQ ID NOs: 18, 107, 121, and 164;
(14) a combination of SEQ ID NOs: 2, 121, 165, and 168;
(15) a combination of SEQ ID NOs: 18, 121, 149, and 165;
(16) a combination of SEQ ID NOs: 13, 18, 121, and 165;
(17) a combination of SEQ ID NOs: 18, 121, 130, 136, and 164;
(18) a combination of SEQ ID NOs: 18, 121, 130, 164, and 314;
(19) a combination of SEQ ID NOs: 18, 114, 121, 130, and 164;
(20) a combination of SEQ ID NOs: 18, 121, 130, 164, and 214;
(21) a combination of SEQ ID NOs: 18, 121, 130, 164, and 193;
(22) a combination of SEQ ID NOs: 18, 121, 130, 164, and 320;
(23) a combination of SEQ ID NOs: 18, 121, 130, 164, and 301;
(24) a combination of SEQ ID NOs: 18, 121, 130, 144, and 164;
(25) a combination of SEQ ID NOs: 18, 121, 130, 164, and 168;
(26) a combination of SEQ ID NOs: 18, 121, 130, 164, and 205;
(27) a combination of SEQ ID NOs: 18, 121, 130, 158, and 164;
(28) a combination of SEQ ID NOs: 18, 121, 130, 164, and 260;
(29) a combination of SEQ ID NOs: 18, 106, 121, 130, and 164;
(30) a combination of SEQ ID NOs: 18, 121, 130, 164, and 318;
(31) a combination of SEQ ID NOs: 18, 121, 130, 164, and 286;
(32) a combination of SEQ ID NOs: 18, 121, 130, 164, and 315;
(33) a combination of SEQ ID NOs: 18, 121, 130, 164, and 237;
(34) a combination of SEQ ID NOs: 18, 121, 130, 164, and 184;
(35) a combination of SEQ ID NOs: 18, 121, 130, 164, and 270;
(36) a combination of SEQ ID NOs: 18, 121, 130, 164, and 309;
(37) a combination of SEQ ID NOs: 18, 121, 130, 164, and 278;
(38) a combination of SEQ ID NOs: 18, 82, 121, 130, and 164;
(39) a combination of SEQ ID NOs: 18, 23, 121, 130, and 164;
(40) a combination of SEQ ID NOs: 18, 121, 130, 164, and 189;
(41) a combination of SEQ ID NOs: 18, 121, 130, 152, and 164;
(42) a combination of SEQ ID NOs: 18, 121, 130, 164, and 213;
(43) a combination of SEQ ID NOs: 18, 121, 130, 164, and 229;
(44) a combination of SEQ ID NOs: 18, 57, 121, 130, and 164;
(45) a combination of SEQ ID NOs: 18, 121, 130, 142, and 164;
(46) a combination of SEQ ID NOs: 18, 121, 130, 155, and 164;
(47) a combination of SEQ ID NOs: 18, 39, 121, 130, and 164;
(48) a combination of SEQ ID NOs: 18, 33, 121, 130, and 164;
(49) a combination of SEQ ID NOs: 18, 121, 126, 130, and 164;
(50) a combination of SEQ ID NOs: 18, 121, 130, 164, and 319;
(51) a combination of SEQ ID NOs: 18, 22, 121, 130, and 164;
(52) a combination of SEQ ID NOs: 18, 59, 121, 130, and 164;
(53) a combination of SEQ ID NOs: 18, 27, 121, 130, and 164;
(54) a combination of SEQ ID NOs: 18, 121, 130, 164, and 201;
(55) a combination of SEQ ID NOs: 18, 19, 121, 130, and 164;
(56) a combination of SEQ ID NOs: 18, 95, 121, 164, and 188; and
(57) a combination of SEQ ID NOs: 13, 18, 121, 130, and 165.

Examples of the combinations of target nucleic acids in the kit or the device for discriminating lung adenocarcinoma patients from test subjects without lung cancer, such as healthy subjects, benign bone and soft tissue tumor patients and benign breast disease patients, or patients having a cancer other than lung cancer, according to the present invention can include combinations of two or more of the above-mentioned polynucleotides consisting of the nucleotide sequences represented by SEQ ID NOs as shown in Table 1. For example, any two or more of the above-mentioned polynucleotides consisting of nucleotide sequences represented by SEQ ID NOs: 1 to 329 can be combined. Among them, at least one polynucleotide of the newly found polynucleotides consisting of the nucleotide sequences represented by SEQ ID NOs: 1 to 163 is preferably selected.

Examples of the combinations of target nucleic acids in the kit or the device for discriminating squamous cell carcinoma patients from test subjects without lung cancer, such as healthy subjects, benign bone and soft tissue tumor patients and benign breast disease patients, or patients having a cancer other than lung cancer, according to the present invention can include combinations of two or more of the above-mentioned polynucleotides consisting of the nucleotide sequences represented by SEQ ID NOs as shown in Table 1. For example, any two or more of the above-mentioned polynucleotides consisting of nucleotide sequences represented by SEQ ID NOs: 1 to 329 can be combined. Among them, at least one polynucleotide of the newly found polynucleotides consisting of the nucleotide sequences represented by SEQ ID NOs: 1 to 163 is preferably selected.

Examples of the combinations of target nucleic acids in the kit or the device for discriminating large cell carcinoma patients from test subjects without lung cancer, such as healthy subjects, benign bone and soft tissue tumor patients and benign breast disease patients, or patients having a cancer other than lung cancer, according to the present invention can include combinations of two or more of the above-mentioned polynucleotides consisting of the nucleotide sequences represented by SEQ ID NOs as shown in Table 1. For example, any two or more of the above-mentioned polynucleotides consisting of nucleotide sequences represented by SEQ ID NOs: 1 to 329 can be combined. Among them, at least one polynucleotide of the newly found polynucleotides consisting of the nucleotide sequences represented by SEQ ID NOs: 1 to 163 is preferably selected.

Examples of the combinations of target nucleic acids in the kit or the device for discriminating small cell carcinoma patients from test subjects without lung cancer, such as healthy subjects, benign bone and soft tissue tumor patients and benign breast disease patients, or patients having a cancer other than lung cancer, according to the present invention can include combinations of two or more of the above-mentioned polynucleotides consisting of the nucleotide sequences represented by SEQ ID NOs as shown in Table 1. For example, any two or more of the above-mentioned polynucleotides consisting of nucleotide sequences represented by SEQ ID NOs: 1 to 329 can be combined. Among them, at least one polynucleotide of the newly found polynucleotides consisting of the nucleotide sequences represented by SEQ ID NOs: 1 to 163 is preferably selected.

The kit or device of the present invention can also comprise polynucleotide(s) which can detect lung cancer and are known in the art or will be found in the future in addition to the polynucleotide(s) (that can comprise variant(s), fragments, or derivative(s)) according to the present invention as described above.

The kit or device of the present invention can also comprise an antibody for measuring a marker or markers for lung cancer examination known in the art, such as CEA and CYFRA21-1, in addition to the polynucleotide(s) according to the present invention as described above.

These polynucleotides and variants thereof or fragments thereof contained in the kit of the present invention may be packaged in different containers either individually or in any combination.

The kit of the present invention may comprise a kit for extracting nucleic acids (e.g., total RNA) from body fluids, cells, or tissues, a fluorescent material for labeling, an enzyme and a medium for nucleic acid amplification, an instruction manual, etc. The device of the present invention is a device for measurement of cancer markers in which nucleic acids such as the polynucleotides according to the present invention described above, variants thereof, derivatives thereof, or fragments thereof are bonded or attached to, for example, a solid phase. Examples of the material for the solid phase include plastics, paper, glass, and silicon. The material for the solid phase is preferably a plastic from the viewpoint of easy processability. The solid phase has any shape and is, for example, square, round, reed-shaped, or film-shaped. The device of the present invention includes, for example, a device for measurement by a hybridization technique. Specific examples thereof include blotting devices and nucleic acid arrays (e.g., microarrays, DNA chips, and RNA chips).

The nucleic acid array technique is a technique which involves bonding or attaching the nucleic acids one by one by use of a method [e.g., a method of spotting the nucleic acids using a high-density dispenser called spotter or arrayer onto the surface of the solid phase surface-treated, if necessary, by coating with L-lysine or the introduction of a functional group such as an amino group or a carboxyl group, a method of spraying the nucleic acids onto the solid phase using an inkjet which injects very small liquid droplets by a piezoelectric element or the like from a nozzle, or a method of sequentially synthesizing nucleotides on the solid phase] to prepare an array such as a chip and measuring target nucleic acids through the use of hybridization using this array.

The kit or the device of the present invention comprises nucleic acids capable of specifically binding to the polynucleotides of at least one, preferably at least two, more preferably at least three, most preferably at least five to all of the lung cancer marker miRNAs, respectively, of the group A described above, or to a polynucleotide(s) consisting of a nucleotide sequence(s) complementary to that of the polynucleotide(s). The kit or the device of the present invention can optionally further comprise nucleic acids capable of specifically binding to the polynucleotides of at least one, preferably at least two, more preferably at least three, most preferably at least five to all of the lung cancer marker miRNAs, respectively, of the group B described above, or to a polynucleotide(s) consisting of a nucleotide sequence(s) complementary to that of the polynucleotide(s).

The kit or the device of the present invention can be used for detecting lung cancer as described in Section 4 below.

### 4. Method for Detecting Lung Cancer

The present invention further provides a method for detecting lung cancer, using the above-mentioned nucleic acid(s) that can be used in the present invention (alternatively, e.g., the kit or the device of the present invention as described in Section 3 above) to measure one or more expression levels of lung cancer-derived genes represented by: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p; and optionally an expression level(s) of lung cancer-derived gene(s) represented by: miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, in a sample, and evaluating in vitro whether or not the subject has lung cancer, based on the expression levels measured (and control expression levels of healthy subjects optionally measured in the same way as above). In the method, for example, using samples, such as blood, serum, or plasma, collected from a subject suspected of having lung cancer and a subject without lung cancer, the expression levels of the above mentioned genes obtained from these subjects are compared, and if the expression level(s) of the target nucleic acid(s) is different between these samples, the subject is evaluated to have lung cancer.

This method of the present invention enables a limitedly invasive, early diagnosis of lung adenocarcinoma, lung squamous cell carcinoma, large cell lung carcinoma, small cell lung carcinoma and other lung cancers with high sensitivity and high specificity and thereby brings about early treatment and improved prognosis. In addition, the disease progression or the effectiveness of surgical, radiotherapeutic, and chemotherapeutic treatments can be monitored by the present invention.

According to the present invention, the method for extracting the lung cancer-derived gene(s) from the sample such as blood, serum, or plasma prepared by the addition of a reagent for RNA extraction in 3D-Gene™ RNA extraction reagent from liquid sample kit (Toray Industries, Inc., Japan) is particularly preferable. A general acidic phenol method (acid guanidinium-phenol-chloroform (AGPC)) or Trizol™ (Life Technologies Corp.) may be used. The lung cancer-derived gene(s) may be also prepared by the addition of a reagent for RNA extraction containing acidic phenol, such as Trizol (Life Technologies Corp.) or Isogen (Nippon Gene Co., Ltd., Japan). Alternatively, a kit such as miRNeasy™ Mini Kit (Qiagen N.V.) may be used, although the method is not limited thereto.

The present invention also provides use of the kit or the device of the present invention for detecting in vitro an expression product(s) of a lung cancer-derived miRNA gene(s) in a sample from a subject.

In the method of the present invention, the kit or the device described above comprises a single polynucleotide or any possible combination of polynucleotides that can be used in the present invention as described above.

In the detection or (genetic) diagnosis of lung cancer according to the present invention, each polynucleotide contained in the kit or the device of the present invention can be used as a probe or a primer. In the case of using the polynucleotides as primers, TaqMan™ MicroRNA Assays from Life Technologies Corp., miScript PCR System from Qiagen N.V., or the like can be used, although the method is not limited thereto.

In the method of the present invention, measurement of the gene expression levels can be performed using the above-mentioned primers or probes according to a routine method in a method known in the art specifically for detecting particular genes, for example, a hybridization technique such as Northern blot, Southern blot, in situ hybridization, Northern hybridization, or Southern hybridization, a quantitative amplification technique such as quantitative RT-PCR, or a method with a next-generation sequencer. A body fluid such as blood, serum, plasma, or urine from a subject is collected as a sample to be assayed according to the type of the detection method used. Alternatively, total RNA prepared from such a body fluid by the method described above may be used, and various polynucleotides including cDNA prepared on the basis of the RNA may be used.

The method, the kit or the device of the present invention is useful for diagnosis of lung cancer or the detection of the presence or absence of lung cancer. Specifically, the detection of lung cancer using the method, the kit or the device can be performed by detecting in vitro an expression level(s) of a gene(s) which is detected by the method or detected using the nucleic acid probe(s) or the primer(s) contained in the kit or the device, in a sample such as blood, serum, plasma, or urine from a subject suspected of having lung cancer. The subject suspected of having lung cancer can be evaluated as having lung cancer when the expression level(s) of a polynucleotide(s) consisting of a nucleotide sequence(s) represented by at least one of, for example, SEQ ID NOs: 1 to 163 and optionally a nucleotide sequence(s) represented by one or more of, for example, SEQ ID NOs: 164 to 329, as target nucleic acids, in the sample such as blood, serum, plasma, or urine of the subject, is significantly high in statistic compared to an expression level(s) of the nucleotide sequences in the sample such as blood, serum, or plasma, or urine of a subject without lung cancer (i.e., also referred to as a control animal).

In the method of the present invention, or the method using the kit or the device of the present invention, the method for detecting the presence or the absence of lung cancer in a sample from a subject comprises collecting a body fluid such as blood, serum, plasma, or urine of a subject, and measuring the expression level(s) of the target gene(s) (or target nucleic acid(s)) contained therein using one or more polynucleotides (including a variant(s), a fragment(s), or a derivative(s)) selected from the groups of polynucleotides of the present invention, to evaluate the presence or absence of lung cancer or to detect lung cancer.

The method for detecting lung cancer according to the present invention can be used in combination with an imaging test method such as chest X-ray examination, CT examination, MRI examination, or PET examination. The method for detecting lung cancer according to the present invention can also be used in combination with sputum cytology, pleural fluid analysis, bronchoscopy, percutaneous needle biopsy or the like, which is a pathological examination method involving the microscopic examination of collected cells or tissues. The method for detecting lung cancer according to the present invention is capable of specifically detecting lung cancer and therefore, can substantially discriminate lung cancer from cancer other than lung cancer and can determine lung cancer with higher reliability by combination with another examination method such as the imaging test method or the pathological examination method described above. Furthermore, the method of the present invention can also be utilized to confirm the necessity of carrying out another examination method such an imaging test or a pathological examination.

The method for detecting lung cancer according to the present invention can also be used to evaluate or diagnose, for example, the presence or absence of amelioration of the disease or the degree of amelioration thereof in a lung cancer patient in the case that a lung cancer-related therapeutic drug which is known or on a development stage (including cisplatin, gefitinib, docetaxel, etoposide, carboplatin, paclitaxel, and combination drugs thereof as non-limiting examples) is administered to the patient for treatment or amelioration of the disease.

The method of the present invention can comprise, for example, the following steps (a), (b), and (c):
(a) a step of contacting in vitro a sample from a subject with a polynucleotide(s) contained in the kit or the device of the present invention;
(b) a step of measuring an expression level(s) of the target nucleic acid(s) in the sample using the polynucleotide(s) as a nucleic acid probe(s) or primer(s); and
(c) a step of evaluating the presence or absence of lung cancer (cells) in the subject on the basis of the measurement results in the step (b).

In one embodiment, the present invention provides a method for detecting lung cancer, comprising: measuring an expression level(s) of a target nucleic acid(s) in a sample of a subject using a nucleic acid(s) capable of specifically binding to at least one, preferably at least two polynucleotides selected from the group consisting of the following miRNAs: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, and miR-92b-3p, or to a polynucleotide(s) consisting of a nucleotide sequence(s) complementary to that of the polynucleotide(s); or a nucleic acid(s) for detecting the polynucleotide(s); and evaluating in vitro whether or not the subject has lung cancer using the above-measured expression levels and control expression levels of a subject(s) without lung cancer measured in the same way as above.

As used herein, the term “evaluating” is evaluation support based on results of in vitro examination, not physician's judgment.

As described above, in the method of the present invention, specifically, miR-6787-5p is hsa-miR-6787-5p, miR-920 is hsa-miR-920, miR-3622a-5p is hsa-miR-3622a-5p, miR-1185-1-3p is hsa-miR-1185-1-3p, miR-4327 is hsa-miR-4327, miR-5739 is hsa-miR-5739, miR-937-5p is hsa-miR-937-5p, miR-1181 is hsa-miR-1181, miR-1185-2-3p is hsa-miR-1185-2-3p, miR-1193 is hsa-miR-1193, miR-1207-5p is hsa-miR-1207-5p, miR-1238-5p is hsa-miR-1238-5p, miR-1246 is hsa-miR-1246, miR-1249-5p is hsa-miR-1249-5p, miR-1292-3p is hsa-miR-1292-3p, miR-1469 is hsa-miR-1469, miR-1470 is hsa-miR-1470, miR-197-5p is hsa-miR-197-5p, miR-208a-5p is hsa-miR-208a-5p, miR-2110 is hsa-miR-2110, miR-211-3p is hsa-miR-211-3p, miR-2467-3p is hsa-miR-2467-3p, miR-3122 is hsa-miR-3122, miR-3141 is hsa-miR-3141, miR-3156-5p is hsa-miR-3156-5p, miR-3158-5p is hsa-miR-3158-5p, miR-3160-5p is hsa-miR-3160-5p, miR-3180-3p is hsa-miR-3180-3p, miR-3191-3p is hsa-miR-3191-3p, miR-3194-3p is hsa-miR-3194-3p, miR-320b is hsa-miR-320b, miR-328-5p is hsa-miR-328-5p, miR-3610 is hsa-miR-3610, miR-3619-3p is hsa-miR-3619-3p, miR-3620-5p is hsa-miR-3620-5p, miR-370-3p is hsa-miR-370-3p, miR-373-5p is hsa-miR-373-5p, miR-3917 is hsa-miR-3917, miR-3937 is hsa-miR-3937, miR-4259 is hsa-miR-4259, miR-4281 is hsa-miR-4281, miR-4294 is hsa-miR-4294, miR-4419b is hsa-miR-4419b, miR-4428 is hsa-miR-4428, miR-4429 is hsa-miR-4429, miR-4433a-3p is hsa-miR-4433a-3p, miR-4447 is hsa-miR-4447, miR-4449 is hsa-miR-4449, miR-4459 is hsa-miR-4459, miR-4480 is hsa-miR-4480, miR-4485-5p is hsa-miR-4485-5p, miR-4486 is hsa-miR-4486, miR-4488 is hsa-miR-4488, miR-4489 is hsa-miR-4489, miR-4505 is hsa-miR-4505, miR-4513 is hsa-miR-4513, miR-4515 is hsa-miR-4515, miR-4530 is hsa-miR-4530, miR-4535 is hsa-miR-4535, miR-4635 is hsa-miR-4635, miR-4640-5p is hsa-miR-4640-5p, miR-4646-5p is hsa-miR-4646-5p, miR-4656 is hsa-miR-4656, miR-4663 is hsa-miR-4663, miR-4665-5p is hsa-miR-4665-5p, miR-4706 is hsa-miR-4706, miR-4707-5p is hsa-miR-4707-5p, miR-4708-3p is hsa-miR-4708-3p, miR-4710 is hsa-miR-4710, miR-4718 is hsa-miR-4718, miR-4722-5p is hsa-miR-4722-5p, miR-4727-3p is hsa-miR-4727-3p, miR-4730 is hsa-miR-4730, miR-4734 is hsa-miR-4734, miR-4740-5p is hsa-miR-4740-5p, miR-4747-3p is hsa-miR-4747-3p, miR-4749-5p is hsa-miR-4749-5p, miR-4755-3p is hsa-miR-4755-3p, miR-4763-5p is hsa-miR-4763-5p, miR-4787-3p is hsa-miR-4787-3p, miR-5008-5p is hsa-miR-5008-5p, miR-5010-5p is hsa-miR-5010-5p, miR-504-3p is hsa-miR-504-3p, miR-5090 is hsa-miR-5090, miR-5100 is hsa-miR-5100, miR-5196-5p is hsa-miR-5196-5p, miR-551b-5p is hsa-miR-551b-5p, miR-557 is hsa-miR-557, miR-5787 is hsa-miR-5787, miR-6090 is hsa-miR-6090, miR-6124 is hsa-miR-6124, miR-6132 is hsa-miR-6132, miR-6510-5p is hsa-miR-6510-5p, miR-6511b-5p is hsa-miR-6511b-5p, miR-6515-3p is hsa-miR-6515-3p, miR-654-5p is hsa-miR-654-5p, miR-658 is hsa-miR-658, miR-668-5p is hsa-miR-668-5p, miR-6722-5p is hsa-miR-6722-5p, miR-6724-5p is hsa-miR-6724-5p, miR-6729-3p is hsa-miR-6729-3p, miR-6737-5p is hsa-miR-6737-5p, miR-6756-5p is hsa-miR-6756-5p, miR-6762-5p is hsa-miR-6762-5p, miR-6763-3p is hsa-miR-6763-3p, miR-6766-5p is hsa-miR-6766-5p, miR-6769a-5p is hsa-miR-6769a-5p, miR-6771-5p is hsa-miR-6771-5p, miR-6786-5p is hsa-miR-6786-5p, miR-6789-5p is hsa-miR-6789-5p, miR-6794-5p is hsa-miR-6794-5p, miR-6796-3p is hsa-miR-6796-3p, miR-6797-5p is hsa-miR-6797-5p, miR-6800-3p is hsa-miR-6800-3p, miR-6802-5p is hsa-miR-6802-5p, miR-6803-5p is hsa-miR-6803-5p, miR-6805-3p is hsa-miR-6805-3p, miR-6805-5p is hsa-miR-6805-5p, miR-6807-5p is hsa-miR-6807-5p, miR-6812-5p is hsa-miR-6812-5p, miR-6819-5p is hsa-miR-6819-5p, miR-6822-5p is hsa-miR-6822-5p, miR-6824-5p is hsa-miR-6824-5p, miR-6826-5p is hsa-miR-6826-5p, miR-6850-5p is hsa-miR-6850-5p, miR-6858-5p is hsa-miR-6858-5p, miR-6861-5p is hsa-miR-6861-5p, miR-6880-3p is hsa-miR-6880-3p, miR-7107-5p is hsa-miR-7107-5p, miR-7109-5p is hsa-miR-7109-5p, miR-7114-5p is hsa-miR-7114-5p, miR-7704 is hsa-miR-7704, miR-7846-3p is hsa-miR-7846-3p, miR-8052 is hsa-miR-8052, miR-8060 is hsa-miR-8060, miR-8071 is hsa-miR-8071, miR-8073 is hsa-miR-8073, miR-874-5p is hsa-miR-874-5p, miR-204-3p is hsa-miR-204-3p, miR-3154 is hsa-miR-3154, miR-3960 is hsa-miR-3960, miR-4433a-5p is hsa-miR-4433a-5p, miR-4455 is hsa-miR-4455, miR-4462 is hsa-miR-4462, miR-4476 is hsa-miR-4476, miR-4508 is hsa-miR-4508, miR-4687-3p is hsa-miR-4687-3p, miR-4687-5p is hsa-miR-4687-5p, miR-4732-5p is hsa-miR-4732-5p, miR-4771 is hsa-miR-4771, miR-642a-3p is hsa-miR-642a-3p, miR-6732-5p is hsa-miR-6732-5p, miR-6760-5p is hsa-miR-6760-5p, miR-6799-5p is hsa-miR-6799-5p, miR-6820-5p is hsa-miR-6820-5p, miR-6821-5p is hsa-miR-6821-5p, miR-6829-5p is hsa-miR-6829-5p, miR-6893-5p is hsa-miR-6893-5p, miR-7108-3p is hsa-miR-7108-3p, miR-7111-5p is hsa-miR-7111-5p, miR-8089 is hsa-miR-8089, miR-885-3p is hsa-miR-885-3p, and miR-92b-3p is hsa-miR-92b-3p.

Additionally, in one embodiment, the nucleic acid(s) (e.g., a probe(s) or a primer(s)) in the method of the present invention is selected from the group consisting of, for example, the following polynucleotides (a) to (e):
(a) a polynucleotide consisting of a nucleotide sequence represented by any of SEQ ID NOs: 1 to 163, or a nucleotide sequence derived from the nucleotide sequence in which the nucleic acid u is replaced with t, a variant thereof, a derivative thereof, or a fragment thereof comprising 15 or more consecutive nucleotides;
(b) a polynucleotide comprising a nucleotide sequence represented by any of SEQ ID NOs: 1 to 163;
(c) a polynucleotide consisting of a nucleotide sequence complementary to a nucleotide sequence represented by any of SEQ ID NOs: 1 to 163, or a nucleotide sequence derived from the nucleotide sequence in which the nucleic acid u is replaced with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 163, or a nucleotide sequence derived from the nucleotide sequence in which the nucleic acid u is replaced with t; and
(e) a polynucleotide hybridizing under stringent conditions to any of the polynucleotides (a) to (d).

The nucleic acid(s) used in the method of the present invention can further comprise a nucleic acid(s) capable of specifically binding to at least one polynucleotide selected from the group consisting of the following miRNAs: miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, or to a polynucleotide consisting of a nucleotide sequence complementary to that of the polynucleotide.

Specifically, miR-1343-3p is hsa-miR-1343-3p, miR-6746-5p is hsa-miR-6746-5p, miR-422a is hsa-miR-422a, miR-187-5p is hsa-miR-187-5p, miR-4632-5p is hsa-miR-4632-5p, miR-6791-5p is hsa-miR-6791-5p, miR-103a-3p is hsa-miR-103a-3p, miR-107 is hsa-miR-107, miR-1199-5p is hsa-miR-1199-5p, miR-1225-3p is hsa-miR-1225-3p, miR-1225-5p is hsa-miR-1225-5p, miR-1228-5p is hsa-miR-1228-5p, miR-1229-5p is hsa-miR-1229-5p, miR-1233-5p is hsa-miR-1233-5p, miR-1237-5p is hsa-miR-1237-5p, miR-1247-3p is hsa-miR-1247-3p, miR-1249-3p is hsa-miR-1249-3p, miR-1254 is hsa-miR-1254, miR-1260b is hsa-miR-1260b, miR-1268a is hsa-miR-1268a, miR-1268b is hsa-miR-1268b, miR-1273g-3p is hsa-miR-1273g-3p, miR-128-1-5p is hsa-miR-128-1-5p, miR-128-2-5p is hsa-miR-128-2-5p, miR-1290 is hsa-miR-1290, miR-150-3p is hsa-miR-150-3p, miR-17-3p is hsa-miR-17-3p, miR-1908-5p is hsa-miR-1908-5p, miR-1909-3p is hsa-miR-1909-3p, miR-1914-3p is hsa-miR-1914-3p, miR-1915-3p is hsa-miR-1915-3p, miR-191-5p is hsa-miR-191-5p, miR-22-3p is hsa-miR-22-3p, miR-23b-3p is hsa-miR-23b-3p, miR-24-3p is hsa-miR-24-3p, miR-296-3p is hsa-miR-296-3p, miR-296-5p is hsa-miR-296-5p, miR-3131 is hsa-miR-3131, miR-3162-5p is hsa-miR-3162-5p, miR-3188 is hsa-miR-3188, miR-3196 is hsa-miR-3196, miR-3197 is hsa-miR-3197, miR-320a is hsa-miR-320a, miR-342-5p is hsa-miR-342-5p, miR-3621 is hsa-miR-3621, miR-3648 is hsa-miR-3648, miR-3656 is hsa-miR-3656, miR-365a-5p is hsa-miR-365a-5p, miR-3665 is hsa-miR-3665, miR-3679-5p is hsa-miR-3679-5p, miR-371a-5p is hsa-miR-371a-5p, miR-3940-5p is hsa-miR-3940-5p, miR-423-5p is hsa-miR-423-5p, miR-4257 is hsa-miR-4257, miR-4270 is hsa-miR-4270, miR-4271 is hsa-miR-4271, miR-4286 is hsa-miR-4286, miR-4298 is hsa-miR-4298, miR-4417 is hsa-miR-4417, miR-4442 is hsa-miR-4442, miR-4446-3p is hsa-miR-4446-3p, miR-4448 is hsa-miR-4448, miR-4454 is hsa-miR-4454, miR-4467 is hsa-miR-4467, miR-4472 is hsa-miR-4472, miR-4507 is hsa-miR-4507, miR-4516 is hsa-miR-4516, miR-451a is hsa-miR-451a, miR-4649-5p is hsa-miR-4649-5p, miR-4651 is hsa-miR-4651, miR-4665-3p is hsa-miR-4665-3p, miR-4674 is hsa-miR-4674, miR-4675 is hsa-miR-4675, miR-4689 is hsa-miR-4689, miR-4695-5p is hsa-miR-4695-5p, miR-4697-5p is hsa-miR-4697-5p, miR-4725-3p is hsa-miR-4725-3p, miR-4739 is hsa-miR-4739, miR-4745-5p is hsa-miR-4745-5p, miR-4763-3p is hsa-miR-4763-3p, miR-4792 is hsa-miR-4792, miR-486-3p is hsa-miR-486-3p, miR-5001-5p is hsa-miR-5001-5p, miR-5195-3p is hsa-miR-5195-3p, miR-550a-5p is hsa-miR-550a-5p, miR-5698 is hsa-miR-5698, miR-6075 is hsa-miR-6075, miR-6088 is hsa-miR-6088, miR-6089 is hsa-miR-6089, miR-6125 is hsa-miR-6125, miR-6126 is hsa-miR-6126, miR-614 is hsa-miR-614, miR-615-5p is hsa-miR-615-5p, miR-619-5p is hsa-miR-619-5p, miR-638 is hsa-miR-638, miR-642b-3p is hsa-miR-642b-3p, miR-650 is hsa-miR-650, miR-663a is hsa-miR-663a, miR-663b is hsa-miR-663b, miR-6717-5p is hsa-miR-6717-5p, miR-6721-5p is hsa-miR-6721-5p, miR-6726-5p is hsa-miR-6726-5p, miR-6727-5p is hsa-miR-6727-5p, miR-6738-5p is hsa-miR-6738-5p, miR-6741-5p is hsa-miR-6741-5p, miR-6749-5p is hsa-miR-6749-5p, miR-6752-5p is hsa-miR-6752-5p, miR-675-5p is hsa-miR-675-5p, miR-6757-5p is hsa-miR-6757-5p, miR-6763-5p is hsa-miR-6763-5p, miR-6765-5p is hsa-miR-6765-5p, miR-6775-5p is hsa-miR-6775-5p, miR-6780b-5p is hsa-miR-6780b-5p, miR-6782-5p is hsa-miR-6782-5p, miR-6784-5p is hsa-miR-6784-5p, miR-6800-5p is hsa-miR-6800-5p, miR-6806-5p is hsa-miR-6806-5p, miR-6840-3p is hsa-miR-6840-3p, miR-6848-5p is hsa-miR-6848-5p, miR-6851-5p is hsa-miR-6851-5p, miR-6870-5p is hsa-miR-6870-5p, miR-6872-3p is hsa-miR-6872-3p, miR-6875-5p is hsa-miR-6875-5p, miR-6877-5p is hsa-miR-6877-5p, miR-6879-5p is hsa-miR-6879-5p, miR-6880-5p is hsa-miR-6880-5p, miR-6885-5p is hsa-miR-6885-5p, miR-6887-5p is hsa-miR-6887-5p, miR-7108-5p is hsa-miR-7108-5p, miR-711 is hsa-miR-711, miR-7113-3p is hsa-miR-7113-3p, miR-744-5p is hsa-miR-744-5p, miR-760 is hsa-miR-760, miR-7845-5p is hsa-miR-7845-5p, miR-7847-3p is hsa-miR-7847-3p, miR-7977 is hsa-miR-7977, miR-8059 is hsa-miR-8059, miR-8063 is hsa-miR-8063, miR-8072 is hsa-miR-8072, miR-874-3p is hsa-miR-874-3p, miR-92a-2-5p is hsa-miR-92a-2-5p, miR-92b-5p is hsa-miR-92b-5p, miR-940 is hsa-miR-940, miR-1228-3p is hsa-miR-1228-3p, miR-1275 is hsa-miR-1275, miR-1307-3p is hsa-miR-1307-3p, miR-1343-5p is hsa-miR-1343-5p, miR-23a-3p is hsa-miR-23a-3p, miR-29b-3p is hsa-miR-29b-3p, miR-3135b is hsa-miR-3135b, miR-3185 is hsa-miR-3185, miR-4532 is hsa-miR-4532, miR-4690-5p is hsa-miR-4690-5p, miR-4758-5p is hsa-miR-4758-5p, miR-4783-3p is hsa-miR-4783-3p, miR-6131 is hsa-miR-6131, miR-625-3p is hsa-miR-625-3p, miR-6511a-5p is hsa-miR-6511a-5p, miR-6765-3p is hsa-miR-6765-3p, miR-6816-5p is hsa-miR-6816-5p, miR-6825-5p is hsa-miR-6825-5p, miR-6845-5p is hsa-miR-6845-5p, miR-7150 is hsa-miR-7150, miR-7641 is hsa-miR-7641, miR-7975 is hsa-miR-7975, and miR-92a-3p is hsa-miR-92a-3p.

In one embodiment, the nucleic acid(s) may further be selected from, for example, the group consisting of the following polynucleotides (f) to (j):
(f) a polynucleotide consisting of a nucleotide sequence represented by any of SEQ ID NOs: 164 to 329, or a nucleotide sequence derived from the nucleotide sequence in which the nucleic acid u is replaced with t, a variant thereof, a derivative thereof, or a fragment thereof comprising 15 or more consecutive nucleotides;
(g) a polynucleotide comprising a nucleotide sequence represented by any of SEQ ID NOs: 164 to 329;
(h) a polynucleotide consisting of a nucleotide sequence complementary to a nucleotide sequence represented by any of SEQ ID NOs: 164 to 329, or a nucleotide sequence derived from the nucleotide sequence in which the nucleic acid u is replaced with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 164 to 329, or a nucleotide sequence derived from the nucleotide sequence in which the nucleic acid u is replaced with t; and
(j) a polynucleotide hybridizing under stringent conditions to any of the polynucleotides (f) to (i).

Examples of the sample used in the method of the present invention can include samples prepared from living tissues (preferably lung tissues) or body fluids such as blood, serum, plasma, and urine from subjects. Specifically, for example, an RNA-containing sample prepared from the tissue, a polynucleotide-containing sample further prepared therefrom, a body fluid such as blood, serum, plasma, or urine, a portion or the whole of a living tissue collected from the subject by biopsy or the like, or a living tissue excised by surgery can be used, and the sample for measurement can be prepared therefrom.

As used herein, the subject refers to a mammal, for example, a human, a monkey, a mouse, or a rat, without any limitation, and is preferably a human.

The steps of the method of the present invention can be changed according to the type of the sample to be measured.

In the case of using RNA as an analyte, the method for detecting lung cancer (cells) can comprise, for example, the following steps (a), (b), and (c):
(a) a step of binding RNA prepared from a sample from a subject (wherein, for example, the 3′ end of the RNA may be polyadenylated for quantitative RT-PCR in step (b)) or complementary polynucleotides (cDNAs) transcribed from the RNA to a polynucleotide(s) in the kit of the present invention;
(b) a step of measuring the sample-derived RNA or the cDNAs synthesized from the RNA, which is/are bound to the polynucleotide(s), by hybridization using the polynucleotide(s) as a nucleic acid probe(s) or by quantitative RT-PCR using the polynucleotide(s) as a primer(s); and
(c) a step of evaluating the presence or absence of lung cancer (or lung cancer-derived gene) on the basis of the measurement results of the step (b).

For example, various hybridization methods can be used for measuring the expression level(s) of a target gene(s), or detecting, examining, evaluating, or diagnosing lung cancer (or lung cancer-derived gene) in vitro according to the present invention. For example, Northern blot, Southern blot, DNA chip analysis, in situ hybridization, Northern hybridization, or Southern hybridization can be used as such a hybridization method. PCR such as quantitative RT-PCR can also be used in combination with hybridization method, or as an alternative thereof.

In the case of using the Northern blot, the presence or absence of expression of each gene or the expression level thereof in the RNA can be detected or measured by use of, for example, the nucleic acid probe(s) that can be used in the present invention. Specific examples thereof can include a method which comprises labeling the nucleic acid probe (or a complementary strand) with a radioisotope (32P, 33P, 35S, etc.), a fluorescent material, or the like, hybridizing the labeled product with the tissue-derived RNA from a subject, which is transferred to a nylon membrane or the like according to a routine method, and then detecting and measuring a signal derived from the label (radioisotope or fluorescent material) on the formed DNA/RNA duplex using a radiation detector (examples thereof can include BAS-1800 II (Fujifilm Corp.)) or a fluorescence detector (examples thereof can include STORM 865 (GE Healthcare Japan Corp.)).

In the case of using the quantitative RT-PCR, the presence or absence of expression of each gene or the expression level thereof in the RNA can be detected or measured by use of, for example, the primer that can be used in the present invention. Specific examples thereof can include a method which comprises recovering the tissue-derived RNA from a subject, preparing cDNAs according to reverse transcription using 3′-end polyadenylation treatment, specific primers, and the like, hybridizing a pair of primers (consisting of a plus strand and a reverse strand binding to the cDNA) prepared from the kit for detection of the present invention with the cDNA such that the region of each target gene marker can be amplified with the cDNA as a template, and performing PCR according to a routine method to detect the obtained single-stranded or double-stranded DNA. The method for detecting the single-stranded or double-stranded DNA can include a method of performing the PCR using the primers labeled in advance with a radioisotope or a fluorescent material, a method of electrophoresing the PCR product on an agarose gel and staining the double-stranded DNA with ethidium bromide or the like for detection, and a method of transferring the produced single-stranded or double-stranded DNA to a nylon membrane or the like according to a routine method and hybridizing the single-stranded or double-stranded DNA to a labeled nucleic acid probe for detection.

In the case of using the nucleic acid array analysis, an RNA chip or a DNA chip in which the kit or device for detection of the present invention is attached as nucleic acid probes (single-stranded or double-stranded) to a substrate (solid phase), for example, is used. Regions having the attached nucleic acid probes are referred to as probe spots, and regions having no attached nucleic acid probe are referred to as blank spots. A group of genes immobilized on a solid-phase substrate is generally called a nucleic acid chip, a nucleic acid array, a microarray, or the like. The DNA or RNA array includes a DNA or RNA macroarray and a DNA or RNA microarray. In the present specification, the term “chip” includes these arrays. 3D-Gene™ Human miRNA Oligo chip (Toray Industries, Inc., Japan) can be used as the DNA chip, though the DNA chip is not limited thereto.

Examples of the measurement using the DNA chip can include, but are not limited to, a method of detecting and measuring a signal derived from the label on the kit or device for detection using an image detector (examples thereof can include Typhoon 9410 (GE Healthcare) and 3D-Gene™ scanner (Toray Industries, Inc., Japan)).

The “stringent conditions” used herein are, as mentioned above, conditions under which a nucleic acid probe hybridizes to its target sequence to a detectably larger extent (e.g., a measurement value equal to or larger than “(a mean of background measurement values)+(a standard error of the background measurement values)×2”) than that for other sequences.

The stringent conditions are defined by hybridization and subsequent washing. Examples of the hybridization conditions include, but not limited to, 30° C. to 60° C. for 1 to 24 hours in a solution containing SSC, a surfactant, formamide, dextran sulfate, a blocking agent(s), etc. In this context, 1×SSC is an aqueous solution (pH 7.0) containing 150 mM sodium chloride and 15 mM sodium citrate. The surfactant includes, for example, SDS (sodium dodecyl sulfate), Triton, or Tween. The hybridization conditions more preferably comprise 3-10×SSC and 0.1-1% SDS. Examples of the conditions for the washing, following the hybridization, which is another condition to define the stringent conditions, can include conditions comprising continuous washing at 30° C. in a solution containing 0.5×SSC and 0.1% SDS, at 30° C. in a solution containing 0.2×SSC and 0.1% SDS, and at 30° C. in a 0.05×SSC solution. It is desirable that the complementary strand should maintain its hybridized state with a target plus strand even by washing under such conditions. Specifically, examples of such a complementary strand can include a strand consisting of a nucleotide sequence in a completely complementary relationship with the nucleotide sequence of the target plus (+) strand, and a strand consisting of a nucleotide sequence having at least 80%, preferably at least 85%, more preferably at least 90% or at least 95% identity to the strand.

Other examples of the “stringent conditions” for the hybridization are described in, for example, Sambrook, J. & Russel, D., Molecular Cloning, A LABORATORY MANUAL, Cold Spring Harbor Laboratory Press, published on Jan. 15, 2001, Vol. 1, 7.42 to 7.45 and Vol. 2, 8.9 to 8.17, and can be used in the present invention.

Examples of the conditions for carrying out PCR using polynucleotide fragments in the kit of the present invention as primers include treatment for approximately 15 seconds to 1 minute at 5 to 10° C. plus a Tm value calculated from the sequences of the primers, using a PCR buffer having composition such as 10 mM Tris-HCL (pH 8.3), 50 mM KCL, and 1 to 2 mM MgCl₂. Examples of the method for calculating such a Tm value include Tm value=2×(the number of adenine residues+the number of thymine residues)+4×(the number of guanine residues+the number of cytosine residues).

In the case of using the quantitative RT-PCR, a commercially available kit for measurement specially designed for quantitatively measuring miRNA, such as TaqMan™ MicroRNA Assays (Life Technologies Corp.), LNA™-based MicroRNA PCR (Exiqon), or Ncode™ miRNA qRT-PCT kit (Invitrogen Corp.) may be used.

In the method of the present invention, measurement of the gene expression level(s) may be performed with a sequencer, in addition to hybridization methods described above. In use of a sequencer, any of DNA sequencers of the first generation based on Sanger method, the second generation with shorter read size, and the third generation with longer read size can be used (herein referred to as “next-generation sequencer”, including sequencers of the second generation and the third generation). For example, a commercially available measurement kit specifically designed for measuring miRNA using Miseq, Hiseq, or NexSeq (Illumina, Inc.); Ion Proton, Ion PGM, or Ion S5/S5 XL (Thermo Fisher Scientific Inc.); PacBio RS II or Sequel (Pacific Biosciences of California, Inc.); MinION (Oxford Nanopore Technologies Ltd.) exemplified in use of a Nanopore sequencer; or the like may be used.

Next-generation sequencing is a method of obtaining sequence information using a next-generation sequencer, and characterized by being capable of simultaneously performing a huge number of sequencing reactions compared to Sanger method (e.g., Rick Kamps et al., Int. J. Mol. Sci., 2017, 18(2), p. 308 and Int. Neurourol. J., 2016, 20 (Suppl.2), S76-83). Examples of next-generation sequencing steps for miRNA include, but not limited to, the following steps: at first, adaptor sequences having predetermined nucleotide sequences are attached, and all RNAs are reverse-transcribed into cDNAs before or after attachment of the sequences. After the reverse transcription, cDNAs derived from specific target miRNAs may be enriched or concentrated by PCR or the like or with a probe or the like, for analyzing the target miRNA before sequencing steps. Subsequent sequencing steps varies in detail depending on the type of a next-generation sequencer, but typically, a sequencing reaction is performed by linking to a substrate via an adaptor sequence and further using the adaptor sequence as a priming site. See details of the sequencing reaction, for example, in Rick Kamps et al. (see supra). Finally, the data are outputted. This step provides a collection of sequence information (reads) obtained by the sequencing reaction. For example, next-generation sequencing can identify a target miRNA(s) based on the sequence information, and measure the expression level thereof based on the number of reads having the sequences of the target miRNA(s).

For the calculation of gene expression levels, statistical treatment described in, for example, Statistical analysis of gene expression microarray data (Speed T., Chapman and Hall/CRC), and A beginner's guide Microarray gene expression data analysis (Causton H. C. et al., Blackwell publishing) can be used in the present invention, though the calculation method is not limited thereto. For example, twice, preferably three times, more preferably six times the standard deviation of the measurement values of the blank spots are added to the average measurement value of the blank spots on the DNA chip, and probe spots having a signal value equal to or larger than the resulting value can be regarded as detected spots. Alternatively, the average measurement value of the blank spots is regarded as a background and can be subtracted from the measurement values of the probe spots to determine gene expression levels. A missing value for a gene expression level can be excluded from the analyte, preferably replaced with the smallest value of the gene expression level in each DNA chip, or more preferably replaced with a value obtained by subtracting 0.1 from a logarithmic value of the smallest value of the gene expression level. In order to eliminate low-signal genes, only genes that show gene expression levels of 2⁶, preferably 2⁸, more preferably 2¹⁰ or larger in 20% or more, preferably 50% or more, more preferably 80% or more of the number of measurement samples can be selected as the analyte. Examples of the normalization of the gene expression level include, but are not limited to, global normalization and quantile normalization (Bolstad, B. M. et al., 2003, Bioinformatics, Vol. 19, p. 185-193).

The present invention also provides a method of detecting a lung cancer (or assisting detection thereof) in a subject, comprising measuring target genes or gene expression levels in a sample from the subject using the gene markers (or target nucleic acids) of the present invention, the nucleic acids (or polynucleotides for detection or diagnosis), the kit, or the device (e.g., chip) for detecting the gene marker or a combination thereof; and assigning the expression levels of the target genes in a sample from the subject to a discriminant (discriminant function), which is prepared using gene expression levels of a sample(s) from a subject(s) (for example, a patient(s)) known to have a lung cancer and a sample(s) from a subject(s) (also referred to as control animal) having no lung cancer, as a training sample(s), and which can distinguishably discriminate the presence or absence of a lung cancer, thereby evaluating the presence or absence of the lung cancer, for example.

Specifically, the present invention further provides the method comprising a first step of measuring in vitro expression levels of target genes, which are known to determine or evaluate that a subject has a lung cancer and/or not, in multiple samples, using the gene marker (or target nucleic acid) of the present invention, the nucleic acids (or polynucleotides for detection or diagnosis), the kit, the device (e.g., chip) for detecting the gene marker or a combination thereof; a second step of preparing a discriminant with the measurement values of the expression levels of the target genes obtained in the first step as training samples; a third step of measuring in vitro the expression levels of the target genes in a sample from the subject in the same manner as in the first step; and a fourth step of assigning the measurement values of the expression levels of the target genes obtained in the third step to the discriminant obtained in the second step, and determining or evaluating whether the subject has a lung cancer or not on the basis of the results obtained from the discriminant, for example. The above target genes are those that can be detected, for example, by the polynucleotides for detection or diagnosis, the polynucleotides contained in the kit or device, and variants thereof or fragments thereof.

The discriminant herein can be prepared by use of any discriminant analysis method that can create a discriminant that distinguishably discriminate the presence or absence of a lung cancer, such as Fisher's discriminant analysis, nonlinear discriminant analysis based on the Mahalanobis' distance, neural network or Support Vector Machine (SVM), although the analysis method is not limited to these specific examples.

When a clustering boundary is a straight line or a hyperplane, the linear discriminant analysis is a method for determining the belonging of a cluster using Formula 1 as a discriminant. In Formula 1, x represents an explanatory variable, w represents a coefficient of the explanatory variable, and w₀ represents a constant term.

$$\begin{matrix}[ Expression 1 ] & \\ f ( x ) = {w}_{0} + {\sum }_{i = 1}^{n} {w}_{i} {x}_{i} & Formula 1\end{matrix}$$

Values obtained from the discriminant are referred to as discriminant scores. The measurement values of a newly offered data set can be assigned as explanatory variables to the discriminant to determine clusters by the signs of the discriminant scores.

The Fisher's discriminant analysis, a type of linear discriminant analysis, is a dimension-reducing method for selecting a dimension suitable for discriminating classes, and constructs a highly discriminating synthetic variable by focusing on the variance of the synthetic variables and minimizing the variance of data having the same label (Venables, W. N. et al., Modern Applied Statistics with S. Fourth edition. Springer, 2002). In the Fisher's discriminant analysis, direction w of projection is determined so as to maximize Formula 2. In this formula, μ represents an average input, ng represents the number of data belonging to class g, and μg represents an average input of the data belonging to class g. The numerator and the denominator are the interclass variance and the intraclass variance, respectively, when each of data is projected in the direction of the vector w. Discriminant coefficient wᵢ is determined by maximizing this ratio (Takafumi Kanamori et al., “Pattern Recognition”, KYORITSU SHUPPAN CO., LTD. (Tokyo, Japan) (2009); Richard O. et al., Pattern Classification, Second Edition., Wiley-Interscience, 2000).

$$\begin{matrix}[ Expression 2 ] & \\ J ( w ) = \frac{{\sum }_{g = 1}^{G} {n}_{g} ( {w}^{T} μ {g}_{} - {w}^{T} μ ) {( {w}^{T} μ {g}_{} - {w}^{T} μ )}^{T}}{{\sum }_{g = 1}^{G} {\sum }_{1 : {y}_{i} = g} ( {w}^{T} x {i}_{} - {w}^{T} {μ}_{g} ) ( {w}^{T} x {i}_{} - {w}^{T} {μ}_{g} )} \\ subject to μ = {\sum }_{i = 1}^{n} \frac{{x}_{i}}{n} , {μ}_{g} = {\sum }_{1 : {u}_{i} = g}^{n} \frac{{x}_{i}}{{n}_{g}} & Formula 2\end{matrix}$$

The Mahalanobis' distance is calculated according to Formula 3 in consideration of data correlation and can be used as nonlinear discriminant analysis for determining a cluster in which a data point belongs to, based on a short Mahalanobis' distance from the data point to that cluster. In Formula 3, μ represents a central vector of each cluster, and S⁻¹ represents an inverse matrix of the variance-covariance matrix of the cluster. The central vector is calculated from explanatory variable x, and an average vector, a median value vector, or the like can be used.

$$\begin{matrix}[ Expression 3 ] & \\ D ( x , μ ) = {\{ {( x - μ )}^{t} {S}^{- 1} ( x - μ ) \}}^{\frac{1}{2}} & Formula 3\end{matrix}$$

SVM is a discriminant analysis method devised by V. Vapnik (The Nature of Statistical Leaning Theory, Springer, 1995). Particular data points of a data set having known classes are defined as explanatory variables, and classes are defined as objective variables. A boundary plane called hyperplane for correctly classifying the data set into the known classes is determined, and a discriminant for data classification is determined using the boundary plane. Then, the measurement values of a newly offered data set can be assigned as explanatory variables to the discriminant to determine classes. In this respect, the result of the discriminant analysis may be classes, may be a probability of being classified into correct classes, or may be a distance from the hyperplane. In SVM, a method of nonlinearly converting a feature vector to a high dimension and performing linear discriminant analysis in the space is known as a method for tackling nonlinear problems. An expression in which an inner product of two factors in a nonlinearly mapped space is expressed only by inputs in their original spaces is called kernel. Examples of the kernel can include a linear kernel, a RBF (Radial Basis Function) kernel, and a Gaussian kernel. While highly dimensional mapping is performed according to the kernel, the optimum discriminant, i.e., a discriminant, can be actually constructed by mere calculation according to the kernel, which avoids calculating features in the mapped space (e.g., Hideki Aso et al., Frontier of Statistical Science 6 “Statistics of pattern recognition and learning—New concepts and approaches”, Iwanami Shoten, Publishers (Tokyo, Japan) (2004); Nello Cristianini et al., Introduction to SVM, Kyoritsu Shuppan Co., Ltd. (Tokyo, Japan) (2008)).

C-support vector classification (C-SVC), a type of SVM, comprises preparing a hyperplane by training a data set with the explanatory variables of two groups and classifying an unknown data set into either of the groups (C. Cortes et al., 1995, Machine Learning, Vol. 20, p. 273-297).

Exemplary calculation of the C-SVC discriminant that can be used in the method of the present invention will be given below. First, all subjects are divided into two groups, i.e., a group of lung cancer patients and a group of test subjects having no lung cancer. For example, lung tissue examination can be used for a reference under which each subject is confirmed to have a lung cancer or not.

Next, a data set consisting of comprehensive gene expression levels of serum-derived samples of the two divided groups (hereinafter, this data set is referred to as a training cohort) is prepared, and a C-SVC discriminant is determined by using genes found to differ clearly in their gene expression levels between the two groups as explanatory variables and this grouping as objective variables (e.g., −1 and +1). An optimizing objective function is represented by Formula 4 wherein e represents all input vectors, y represents an objective variable, a represents a Lagrange's undetermined multiplier vector, Q represents a positive definite matrix, and C represents a parameter for adjusting constrained conditions.

$$\begin{matrix}[ Expression 4 ] & \\ {min}_{a} \frac{1}{2} {a}^{T} Q a - {e}^{T} a subject to {y}^{T} a = 0 , 0 \leq {a}_{i} \leq C , i = 1 , … , l , & Formula 4\end{matrix}$$

Formula 5 is a finally obtained discriminant, and a group in which the data point belongs to can be determined on the basis of the sign of a value obtained according to the discriminant. In this formula, x represents a support vector, y represents a label indicating the belonging of a group, a represents the corresponding coefficient, b represents a constant term, and K represents a kernel function.

$$\begin{matrix}[ Expression 5 ] & \\ f ( x ) = sgn ( {\sum }_{i = 1}^{l} {y}_{i} {a}_{i} K ( {x}_{i} , x ) + b ) & Formula 5\end{matrix}$$

For example, a RBF kernel defined by Formula 6 can be used as the kernel function. In this formula, x represents a support vector, and y represents a kernel parameter for adjusting the complexity of the hyperplane.

$$\begin{matrix}[ Expression 6 ] & \\ K ( {x}_{i} , {x}_{j} ) = exp ( - r {‖ {x}_{i} - {x}_{j} ‖}^{2} ) , r < 0 & Formula 6\end{matrix}$$

In addition, an approach such as neural network, k-nearest neighbor algorithms, decision trees, or logistic regression analysis can be selected as a method for determining or evaluating the presence or absence of lung cancer in a sample from a subject.

In an embodiment, the method of the present invention can comprise, for example, the following steps (a), (b) and (c):
(a) a step of measuring an expression level(s) of a target gene(s) in samples from subjects who are already known to have lung cancer or known to have no lung cancer, using the polynucleotide(s), the kit, or the device (e.g., DNA chip) for detection or diagnosis according to the present invention;
(b) a step of preparing the discriminants of Formulas 1 to 3, 5 and 6 described above from the measurement values of the expression level determined in the step (a), and
(c) a step of measuring an expression level(s) of the target gene(s) in a sample from a subject using the polynucleotide(s), the kit, or the device (e.g., DNA chip) for detection or diagnosis according to the present invention, and assigning the obtained measurement value(s) to the discriminants prepared in the step (b), and determining or evaluating that the subject has a lung cancer or not on the basis of the obtained results, or evaluating the expression level derived from a lung cancer patient by comparison with a control from a subject having no lung cancer (including, e.g., a healthy subject). In this context, in the discriminants of Formulas 1 to 3, 5 and 6, x represents an explanatory variable and includes a value obtained by measuring a polynucleotide(s) or a fragment(s) thereof selected from the polynucleotides serving as target nucleic acids described in Section 2 above. Specifically, the explanatory variable of the present invention for discriminating a lung cancer patient and a subject having no lung cancer is a gene expression level(s) selected from, for example, the following expression level (1) or (2).
(1) a gene expression level(s) in the serum of a lung cancer patient and a subject having no lung cancer measured by any nucleic acid (e.g., DNA or RNA) comprising 15 or more consecutive nucleotides in the nucleotide sequence represented by any of SEQ ID NOs: 1 to 163 or a complementary sequence thereof; and
(2) a gene expression level(s) in the serum of a lung cancer patient and a subject having no lung cancer measured by any nucleic acid (e.g., DNA or RNA) comprising 15 or more consecutive nucleotides in the nucleotide sequence represented by any of SEQ ID NOs: 164 to 329 or a complementary sequence thereof.

As described above, as the method for determining or evaluating whether a subject has a lung cancer or not in a sample from the subject, it is necessary to use a discriminant employing one or more gene expression levels as an explanatory variable(s). In particular, for enhancing the discrimination accuracy of the discriminant using a single gene expression level alone, it is necessary to use a gene having a clear difference in expression level between two groups consisting of a group of lung cancer patients and a group of healthy subjects, in a discriminant.

Specifically, the gene that is used for an explanatory variable of a discriminant is preferably determined as follows. First, using comprehensive gene expression levels of a group of lung cancer patients and comprehensive gene expression levels of a group of test subjects having no lung cancer, both of which are in a training cohort, as a data set, the degree of difference in the expression level of each gene between the two groups is obtained by use of, for example, the P value of a parametric analysis such as t-test, the P value of a nonparametric analysis such as the Mann-Whitney's U test or the P value of the Wilcoxon test.

The gene can be regarded as being statistically significant when the critical rate (significance level) as the P value obtained by the test is smaller than, for example, 5%, 1%, or 0.01%.

In order to correct an increased probability of type I error attributed to the repetition of a test, a method known in the art, for example, Bonferroni or Holm method, can be used for the correction (e.g., Yasushi Nagata et al., “Basics of statistical multiple comparison methods”, Scientist Press Co., Ltd. (Tokyo, Japan) (2007)). As an example of the Bonferroni correction, for example, the P value obtained by a test is multiplied by the number of repetitions of the test, i.e., the number of genes used in the analysis, and the obtained value can be compared with a desired significance level to suppress a probability of causing type I error in the whole test.

Instead of statistical tests, the absolute value of an expression ratio of a median value of each gene expression level (fold change) between gene expression levels of a group of patients having lung cancer and gene expression levels of a group of test subjects having no lung cancer may be calculated to select a gene that is used for an explanatory variable in a discriminant. Alternatively, ROC curves may be prepared using gene expression levels of a group of patients having lung cancer and a group of test subjects having no lung cancer, and a gene that is used for an explanatory variable in a discriminant can be selected on the basis of an AUROC value.

Next, a discriminant that can be calculated by various methods described above is prepared using any number of genes having large difference in their gene expression levels determined here. Examples of the method for constructing a discriminant that produces the largest discrimination accuracy include a method of constructing a discriminant in every combination of genes that satisfy the significance level being P value, and a method of repetitively evaluating the genes for use in the preparation of a discriminant while increasing the number of genes one by one in a descending order of difference in gene expression level (Furey T S. et al., 2000, Bioinformatics., Vol. 16, p. 906-14). To the discriminant, the gene expression level of another independent patient having a lung cancer or a test subject having no lung cancer is assigned as an explanatory variable to calculate discrimination results of the group to which the independent patient having a lung cancer or the test subject having no lung cancer belongs. Specifically, the gene set for diagnosis found and the discriminant constructed using the gene set for diagnosis can be evaluated in an independent sample cohort to find more universal gene set for diagnosis that can detect a lung cancer and a more universal method for discriminating a lung cancer.

In preparing a discriminant using expression levels of multiple genes as an explanatory variable, it is not necessary to select a gene having a clear difference in expression level between the group of lung cancer patients and the group of test subjects having no lung cancer as described above. Specifically, if expression levels of multiple genes are used in combination even though the expression levels of individual genes do not clearly differ, a discriminant having high discriminant performance can be obtained, as the case may be. Because of this, a method of directly searching a discriminant having high discriminant performance without prior selection of the gene to be employed in the discriminant can also be used.

Split-sample method is preferably used for evaluating the performance (generality) of the discriminant. Specifically, a data set is divided into a training cohort and a validation cohort, and gene selection by a statistical test and discriminant preparation are performed using the training cohort. Accuracy, sensitivity, and specificity are calculated using a result of discriminating a validation cohort according to the discriminant, and a true group to which the validation cohort belongs, to evaluate the performance of the discriminant. On the other hand, instead of dividing a data set, the gene selection by a statistical test and discriminant preparation may be performed using all of samples, and accuracy, sensitivity, and specificity can be calculated by the discriminant using a newly prepared sample cohort for evaluation of the performance of the discriminant.

The present invention provides a polynucleotide(s) for detection or diagnosis of a disease useful for diagnosing and treating a lung cancer, a method for detecting a lung cancer using the polynucleotide(s), and a kit and device for detecting or diagnosing a lung cancer, comprising the polynucleotide(s). Particularly, in order to select a gene(s) for diagnosis and prepare a discriminant so as to exhibit accuracy beyond the lung cancer diagnosis method using the existing tumor marker CEA, a gene set for diagnosis and a discriminant for the method of present invention can be constructed, which exhibit accuracy beyond CEA, for example, by comparing expressed genes in serum from a patient confirmed to be negative using CEA but finally found to have a lung cancer by detailed examination such as computed tomography using a contrast medium, with genes expressed in serum from a patient having no lung cancer.

For example, the gene set for diagnosis is set to any combination selected from: one or two or more of the polynucleotides based on a nucleotide sequence(s) represented by any of SEQ ID NOs: 1 to 163 as described above; and optionally one or two or more of the polynucleotides based on a nucleotide sequence(s) represented by any of SEQ ID NOs: 164 to 329. Further, a discriminant is constructed using the expression levels of the gene set for diagnosis in samples from a lung cancer patient as a result of tissue diagnosis and samples from a test subject having no lung cancer as a result of tissue diagnosis. As a result, whether a subject, from which a sample with unknown lung cancer status is provided, has a lung cancer or not can be determined with 100% accuracy at the maximum by measuring expression levels of the gene set for diagnosis in the sample.

### EXAMPLES

The present invention will be described further specifically with reference to Examples below. However, the scope of the present invention is not intended to be limited by these Examples.

### Reference Example

### <Collection of Samples>

Sera were collected using VENOJECT II vacuum blood collecting tube VP-AS109K60 (Terumo Corp. (Japan)) from total 10,869 people (Table 11a) including 4,660 healthy subjects, 327 benign bone and soft tissue tumor patients, 41 benign breast disease patients, 1,694 lung cancer patients, 1,417 stomach cancer patients, 595 esophagus cancer patients, 355 liver cancer patients, 862 pancreatic cancer patients, 406 biliary cancer patients, and 512 bladder cancer patients, after receiving their informed consents. The histological types of the lung cancer patients were adenocarcinoma in 1,308 people, squamous cell carcinoma in 243 people, large cell carcinoma in 23 people, small cell carcinoma in 25 people, adenosquamous carcinoma in 18 people, polymorphic cell cancer in 33 people, salivary gland-type cancer in 2 people, carcinoid tumor in 13 people, preinvasive lesion in 1 person, and other lung cancers in 28 people. Also, the stages of the lung cancer patients were stage IA in 1,068 people, stage IB in 337 people, stage IIA in 97 people, stage IIB in 89 people, stage IIIA in 46 people, stage IIIB in 29 people, stage IV in 4 people, unknown stages in 24 people (Table 2).

### <Extraction of Total RNA>

Total RNA was obtained using a reagent for “RNA extraction in 3D-Gene™ RNA extraction reagent from liquid sample kit” (Toray Industries, Inc. (Japan)) according to the protocol provided by the manufacturer, from 300 μL of the serum sample obtained from each of 10,869 people in total.

### <Measurement of Gene Expression Level>

MicroRNA in the total RNA that was obtained from the serum samples of a total of 10,869 people was fluorescently labeled by use of 3D-Gene™ miRNA Labeling kit (Toray Industries, Inc.) according to the protocol provided by the manufacturer. The oligo DNA chip used was 3D-Gene™ Human miRNA Oligo chip (Toray Industries, Inc.) with attached probes having sequences complementary to 2,565 miRNAs among the miRNAs registered in miRBase Release 21. Hybridization under stringent conditions and washing following the hybridization were performed according to the protocol provided by the manufacturer. The DNA chip was scanned using 3D-Gene™ scanner (Toray Industries, Inc.) to obtain images. Fluorescence intensity was digitized using 3D-Gene™ Extraction (Toray Industries, Inc.). The digitized fluorescence intensity was converted to a logarithmic value having a base of 2 and used as a gene expression level, from which a blank value was subtracted. A missing value was replaced with a signal value 0.1. As a result, the comprehensive gene expression levels of the miRNAs in the sera were obtained for the 10,869 people described above.

Subsequently, the samples were extracted for use in the discriminant analysis of lung cancer. In the description below, healthy subjects, benign bone and soft tissue tumor patients and benign breast disease patients, and patients having a cancer other than lung cancer will be collectively referred to as “test subjects without lung cancer”. Also, stomach cancer, esophagus cancer, liver cancer, pancreatic cancer, biliary cancer and bladder cancer patients will be collectively referred to as “patients having a cancer other than lung cancer”. Firstly, 1,694 lung cancer patients were used as a positive sample group. Secondly, 1,800 people from the healthy subjects described above, and a total of 1,800 people including 300 people having each cancer other than lung cancer were extracted at random, and combined with 368 benign bone and soft tissue tumor patients and benign breast disease patients to select a total of 3,968 people as a negative sample group (Table 11b). Thirdly, 70% of each sample group was used as a training cohort and the remaining 30% thereof as a validation cohort. Specifically, the training cohort consisted of 1,233 healthy subjects, 263 benign bone and soft tissue tumor patients and benign breast disease patients, 1,186 lung cancer patients and 1,281 patients having a cancer other than lung cancer; while the validation cohort consisted of 567 healthy subjects, 105 benign bone and soft tissue tumor patients and benign breast disease patients, 508 lung cancer patients and 519 patients having a cancer other than lung cancer. Calculation and statistical analysis using the digitized gene expression levels of the miRNAs were carried out using R language 3.3.1 (R Core Team (2016). R: A language and environment for statistical computing. R Foundation for Statistical Computing, Vienna, Austria. URL https://www.R-project.org/.) and MASS package 7.3.45 (Venables, W. N. & Ripley, B. D. (2002) Modern Applied Statistics with S. Fourth Edition. Springer, New York. ISBN 0-387-95457-0).

TABLE 2

| | | Stage | | | | | | | | |
|---|---|---|---|---|---|---|---|---|---|---|
| | | IA | IB | IIA | IIB | IIIA | IIIB | IV | Unknown | Total |
| Histological | Adenocarcinoma | 917 | 250 | 57 | 36 | 19 | 20 | 2 | 7 | 1308 |
| type | Squamous cell | 95 | 54 | 30 | 39 | 18 | 6 | 0 | 1 | 243 |
| | carcinoma | | | | | | | | | |
| | Large cell carcinoma | 11 | 5 | 2 | 2 | 2 | 0 | 0 | 1 | 23 |
| | Small cell carcinoma | 7 | 2 | 1 | 0 | 0 | 0 | 0 | 1 | 11 |
| | Combined small | 8 | 3 | 2 | 0 | 0 | 0 | 1 | 0 | 14 |
| | cell carcinoma | | | | | | | | | |
| | Adenosquamous | 10 | 5 | 0 | 3 | 0 | 0 | 0 | 0 | 18 |
| | carcinoma | | | | | | | | | |
| | Polymorphic cell | 6 | 11 | 4 | 6 | 4 | 0 | 1 | 1 | 33 |
| | cancer | | | | | | | | | |
| | Salivary gland-type | 0 | 0 | 0 | 0 | 0 | 1 | 0 | 1 | 2 |
| | cancer | | | | | | | | | |
| | Carcinoid tumor | 3 | 1 | 0 | 0 | 1 | 0 | 0 | 8 | 13 |
| | Preinvasive lesion | 1 | 0 | 0 | 0 | 0 | 0 | 0 | 0 | 1 |
| | Others | 10 | 6 | 1 | 3 | 2 | 2 | 0 | 4 | 28 |
| | Total | 1068 | 337 | 97 | 89 | 46 | 29 | 4 | 24 | 1694 |

TABLE 11

| | | b. | b1. | b2. |
|---|---|---|---|---|
| | | All | Training | Validation |
| | | samples | cohort | cohort |
| | a. | used in | used in | used in |
| | All | discriminant | discriminant | discriminant |
| Subject | samples | analysis | analysis | analysis |
| Healthy | 4660 | 1800 | 1233 | 567 |
| Benign bone and | 327 | 368 | 263 | 105 |
| soft tissue tumor | | | | |
| Benign breast | 41 | | | |
| disease | | | | |
| Lung cancer | 1694 | 1694 | 1186 | 508 |
| Stomach cancer | 1417 | 1800 | 1281 | 519 |
| Esophagus cancer | 595 | | | |
| Liver cancer | 355 | | | |
| Pancreatic cancer | 862 | | | |
| Biliary cancer | 406 | | | |
| Bladder cancer | 512 | | | |

### Example 1

<Discriminant Analysis Using Up to Two miRNAs in Combination>

In this Example, a discriminant(s) was prepared using one or two gene markers in the training cohort including the lung cancer patients and the test subjects without lung cancer (Table 11 b1), and then, the discriminant performance was evaluated in the validation cohort (Table 111b2). Based on the evaluation, gene(s) used in discriminant(s) with high performance were extracted to obtain gene marker(s) that was able to detect lung cancer.

To be more specific, firstly the miRNA expression levels of the training cohort and the validation cohort obtained in the preceding Reference Examples were combined and normalized by global normalization. Secondly, in order to acquire diagnostic markers with higher reliability, only 396 genes having the expression level of 2⁶ or higher in 50% or more of the samples in either of the group of the lung cancer patients or the group of the test subjects without lung cancer were selected as analytes.

Thirdly, one and two in combination of the 396 gene expression level measurement values described above were subjected to the Fisher's discriminant analysis to construct discriminants to discriminate the presence or absence of lung cancer. Accuracy, sensitivity, and specificity in the validation cohort were further calculated using the discriminants prepared above and the discriminant performance was validated using the independent samples. As a result, 645 discriminants with 80% or more discrimination accuracy in the validation cohort were obtained. Among these discriminats, for the discriminants that used a combination of two gene expression levels, only those whose discrimination accuracy is better than the discriminant that used any single one of the gene expression levels were selected, which led to 490 discriminants with 80% or more discrimination accuracy. The 281 genes used in these discriminants were selected as diagnostic markers for the lung cancer patients and the test subjects without lung cancer. In this way, miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-2467-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3619-3p, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4730, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, and miR-940, and the relevant polynucleotides consisting of nucleotide sequences of SEQ ID NOs: 1 to 138 and 164 to 306, were found. Among them, the genes newly found as the markers for examining the presence or absence of lung cancer are polynucleotides consisting of the nucleotide sequences represented by SEQ ID NOs: 1 to 138.

The sensitivities in the validation cohort determined by the discriminants obtained using any single one of the polynucleotides consisting of the nucleotide sequences represented by SEQ ID NOs: 188, 164, 85, 13, 175, 137, 231, 195, 263, 165, 226, 94, 45, 190, 274, 80, 220, 198, 98, 43, 2, 115, 299, 50, 196, 31, 182, 72, 96, 70, 40, 127, 183, 68, 3, 60, 66, 25, 75, 12, 255, 7, 1, 291, 87, 199, 120, 222, 278, 260, 246, 197, 103, 22, 106, 57, 29, 184, 206, 135, 179, 287, 56, 207, 261, 201, 217, 172, 300, 102, 285, 20, 21, 73, 78, 15, 30, 134, 76, 107, 97, 23, 33, 215, 122, 38, 54, 225, 26, 298, 114, 185, 128, 109, 104, 277, 303, 181, 59, 209, 236, 214, 51, 99, 105, 294, 58, 272, 101, 42, 180, 170, 47, 44, 16, 124, 241, 46, 130, 79, 247, 262, 95, 267, 69, 259, 118, 234, 138, 286, 110, 173, 200, 257, 167, 8, 111, 27, 64, 304, 177, 74, 34, 17, 36, 171, 251, 211, 193, and 256 among the polynucleotides described above, are shown in Table 3. Also, discriminant coefficients and constant terms are shown in Table 4. In this context, the general sensitivity of the existing marker CEA has been reported as being 69%. Accordingly, it was demonstrated that the polynucleotides represented by these SEQ ID NOs singly detect lung cancer with sensitivity beyond CEA.

Discriminants that were all able to discriminate lung cancer with 80% or more accuracy were also able to be prepared by combining each of the expression levels of the 281 genes represented by the nucleotide sequences of SEQ ID NOs: 1 to 138 and 164 to 306 with another gene expression level (Table 5). In this respect, discriminant coefficients and constant terms are shown in Table 6. The performance of all the discriminants using these combinations exceeded the discriminant performance of the existing marker CEA. Note that, in the tables, in the column of “SEQ ID NO or Gene”, the combinations of multiple polynucleotides used are described by SEQ ID NOs. (the same applies to tables described later).

From the above, it was demonstrated that all polynucleotides consisting of the nucleotide sequences represented by SEQ ID NOs: 1 to 138 and 164 to 306 are genes capable of discriminating a lung cancer patient from a test subject without lung cancer with high accuracy if these are used alone or in combination of two or more.

TABLE 3

| SEQ | Training cohort | | | Validation cohort | | |
|---|---|---|---|---|---|---|
| ID | Accu- | Sensi- | Speci- | Accu- | Sensi- | Speci- |
| NO: | racy | tivity | ficity | racy | tivity | ficity |
| 188 | 71.9 | 98.1 | 60.8 | 72.7 | 97.0 | 62.3 |
| 164 | 83.4 | 97.0 | 77.7 | 83.8 | 95.9 | 78.6 |
| 85 | 73.2 | 95.3 | 63.8 | 74.0 | 94.3 | 65.4 |
| 13 | 73.4 | 93.4 | 64.9 | 73.0 | 91.7 | 65.1 |
| 175 | 71.3 | 91.4 | 62.7 | 71.5 | 91.5 | 62.9 |
| 137 | 74.9 | 93.9 | 66.8 | 74.4 | 91.1 | 67.3 |
| 231 | 74.4 | 91.8 | 66.9 | 75.8 | 90.2 | 69.6 |
| 319 | 72.2 | 92.6 | 63.5 | 72.7 | 90.2 | 65.2 |
| 195 | 71.1 | 92.2 | 62.0 | 71.9 | 90.2 | 64.1 |
| 263 | 71.5 | 91.0 | 63.2 | 73.0 | 90.0 | 65.7 |
| 165 | 78.4 | 89.7 | 73.6 | 79.7 | 89.8 | 75.4 |
| 226 | 72.0 | 89.5 | 64.5 | 74.5 | 89.6 | 68.0 |
| 94 | 77.3 | 87.6 | 72.9 | 77.3 | 88.8 | 72.5 |
| 45 | 73.3 | 88.9 | 66.6 | 75.0 | 88.8 | 69.1 |
| 190 | 68.7 | 89.0 | 60.0 | 70.3 | 88.4 | 62.6 |
| 274 | 74.2 | 85.7 | 69.2 | 75.0 | 88.2 | 69.4 |
| 328 | 72.1 | 89.4 | 64.7 | 74.4 | 87.2 | 68.9 |
| 80 | 68.9 | 86.8 | 61.2 | 70.6 | 87.0 | 63.6 |
| 220 | 74.1 | 87.9 | 68.2 | 75.5 | 86.8 | 70.7 |
| 198 | 73.1 | 87.5 | 67.0 | 73.7 | 86.6 | 68.2 |
| 98 | 76.2 | 86.6 | 71.7 | 77.6 | 86.4 | 73.8 |
| 43 | 72.0 | 84.2 | 66.7 | 73.7 | 86.4 | 68.3 |
| 316 | 69.2 | 86.0 | 62.1 | 71.5 | 86.4 | 65.2 |
| 2 | 79.7 | 87.4 | 76.4 | 78.3 | 86.2 | 75.0 |
| 115 | 73.4 | 88.0 | 67.2 | 74.5 | 86.0 | 69.5 |
| 299 | 71.7 | 88.0 | 64.7 | 73.2 | 85.8 | 67.8 |
| 50 | 70.4 | 87.4 | 63.2 | 70.9 | 85.8 | 64.6 |
| 150 | 71.6 | 88.5 | 64.4 | 71.0 | 85.6 | 64.8 |
| 196 | 73.0 | 86.9 | 67.1 | 72.8 | 85.2 | 67.5 |
| 31 | 64.1 | 83.6 | 55.7 | 67.3 | 85.2 | 59.7 |
| 182 | 70.0 | 86.0 | 63.2 | 71.8 | 85.0 | 66.2 |
| 72 | 74.0 | 87.0 | 68.5 | 75.5 | 84.8 | 71.5 |
| 318 | 67.8 | 84.7 | 60.5 | 69.3 | 84.8 | 62.6 |
| 149 | 75.5 | 85.8 | 71.2 | 75.0 | 84.6 | 70.9 |
| 312 | 73.1 | 87.4 | 67.0 | 73.8 | 84.6 | 69.2 |
| 96 | 71.4 | 84.3 | 65.9 | 72.7 | 84.3 | 67.8 |
| 329 | 73.7 | 84.1 | 69.3 | 74.7 | 84.1 | 70.7 |
| 70 | 71.4 | 86.5 | 64.9 | 72.1 | 84.1 | 67.0 |
| 40 | 76.4 | 88.7 | 71.2 | 76.4 | 83.7 | 73.3 |
| 127 | 68.7 | 84.5 | 62.0 | 71.3 | 83.3 | 66.2 |
| 153 | 76.4 | 86.1 | 72.3 | 76.8 | 83.1 | 74.1 |
| 183 | 72.9 | 81.8 | 69.1 | 73.9 | 83.1 | 69.9 |
| 148 | 70.6 | 83.5 | 65.1 | 71.7 | 83.1 | 66.9 |
| 68 | 73.3 | 86.4 | 67.7 | 73.3 | 82.9 | 69.2 |
| 3 | 79.2 | 85.7 | 76.4 | 79.7 | 82.7 | 78.4 |
| 60 | 77.8 | 84.4 | 75.0 | 78.2 | 82.5 | 76.4 |
| 66 | 74.8 | 85.5 | 70.2 | 76.3 | 82.5 | 73.7 |
| 25 | 66.3 | 81.6 | 59.8 | 68.6 | 82.5 | 62.6 |
| 75 | 70.7 | 83.8 | 65.1 | 71.8 | 82.3 | 67.3 |
| 12 | 69.2 | 84.7 | 62.6 | 70.5 | 82.3 | 65.4 |
| 255 | 68.9 | 86.3 | 61.5 | 69.6 | 82.3 | 64.2 |
| 7 | 75.4 | 81.6 | 72.8 | 76.7 | 82.1 | 74.4 |
| 1 | 79.4 | 84.7 | 77.1 | 79.4 | 81.9 | 78.3 |
| 291 | 73.3 | 81.6 | 69.7 | 73.9 | 81.9 | 70.4 |
| 162 | 64.6 | 81.5 | 57.4 | 66.3 | 81.9 | 59.6 |
| 163 | 75.3 | 83.0 | 72.1 | 74.7 | 81.7 | 71.8 |
| 87 | 72.6 | 84.2 | 67.6 | 73.3 | 81.7 | 69.7 |
| 199 | 68.4 | 81.3 | 62.9 | 70.9 | 81.7 | 66.2 |
| 120 | 73.7 | 80.9 | 70.6 | 73.3 | 81.5 | 69.9 |
| 222 | 70.2 | 77.2 | 67.3 | 71.3 | 81.5 | 67.0 |
| 311 | 74.5 | 82.6 | 71.0 | 75.2 | 81.3 | 72.5 |
| 278 | 64.5 | 83.7 | 56.4 | 65.7 | 81.3 | 59.1 |
| 260 | 76.7 | 84.3 | 73.4 | 76.5 | 81.1 | 74.6 |
| 246 | 71.6 | 85.2 | 65.8 | 72.1 | 81.1 | 68.3 |
| 197 | 73.7 | 84.1 | 69.3 | 73.9 | 80.9 | 70.9 |
| 103 | 68.4 | 80.9 | 63.1 | 70.1 | 80.9 | 65.5 |
| 22 | 70.3 | 82.6 | 65.1 | 71.4 | 80.7 | 67.4 |
| 106 | 70.9 | 83.2 | 65.7 | 71.6 | 80.5 | 67.8 |
| 322 | 69.4 | 81.3 | 64.3 | 71.5 | 80.5 | 67.7 |
| 57 | 69.1 | 83.5 | 63.0 | 70.9 | 80.5 | 66.8 |
| 309 | 68.2 | 80.9 | 62.7 | 69.6 | 80.5 | 64.9 |
| 29 | 78.1 | 82.6 | 76.2 | 78.2 | 80.1 | 77.4 |
| 184 | 71.5 | 78.2 | 68.6 | 72.2 | 80.1 | 68.8 |
| 206 | 63.7 | 81.3 | 56.2 | 65.0 | 80.1 | 58.5 |
| 135 | 78.9 | 83.3 | 77.0 | 77.9 | 79.9 | 77.1 |
| 179 | 73.5 | 81.5 | 70.0 | 75.0 | 79.9 | 72.9 |
| 287 | 75.9 | 82.5 | 73.1 | 77.1 | 79.5 | 76.1 |
| 56 | 71.9 | 79.9 | 68.5 | 73.9 | 79.5 | 71.5 |
| 207 | 67.0 | 81.8 | 60.7 | 68.9 | 79.5 | 64.3 |
| 261 | 64.4 | 78.8 | 58.3 | 66.2 | 79.5 | 60.5 |
| 201 | 71.8 | 80.4 | 68.1 | 71.6 | 79.1 | 68.3 |
| 217 | 74.7 | 80.6 | 72.2 | 76.3 | 78.9 | 75.1 |
| 317 | 71.9 | 79.0 | 68.9 | 72.7 | 78.9 | 70.0 |
| 172 | 69.4 | 78.4 | 65.6 | 71.3 | 78.9 | 68.1 |
| 300 | 69.6 | 82.7 | 64.1 | 70.7 | 78.7 | 67.3 |
| 102 | 77.1 | 78.6 | 76.5 | 76.0 | 78.5 | 75.0 |
| 285 | 69.5 | 81.0 | 64.6 | 69.9 | 78.5 | 66.2 |
| 20 | 73.8 | 80.1 | 71.2 | 74.3 | 78.3 | 72.5 |
| 159 | 70.9 | 80.7 | 66.8 | 70.6 | 78.3 | 67.3 |
| 21 | 68.4 | 80.2 | 63.4 | 70.5 | 78.1 | 67.2 |
| 73 | 61.6 | 79.0 | 54.2 | 64.0 | 78.1 | 57.9 |
| 78 | 70.5 | 80.9 | 66.1 | 70.9 | 78.0 | 67.8 |
| 15 | 68.6 | 76.5 | 65.2 | 70.5 | 78.0 | 67.3 |
| 30 | 70.3 | 79.8 | 66.3 | 71.3 | 77.8 | 68.5 |
| 134 | 70.6 | 78.7 | 67.2 | 72.9 | 77.6 | 70.9 |
| 315 | 67.4 | 73.5 | 64.8 | 70.6 | 77.6 | 67.6 |
| 76 | 66.5 | 76.5 | 62.2 | 67.3 | 77.6 | 63.0 |
| 107 | 71.3 | 80.2 | 67.5 | 72.5 | 77.2 | 70.4 |
| 97 | 61.8 | 75.8 | 55.8 | 63.0 | 77.0 | 57.0 |
| 23 | 74.8 | 81.2 | 72.0 | 74.7 | 76.8 | 73.9 |
| 33 | 71.7 | 78.5 | 68.7 | 72.0 | 76.4 | 70.2 |
| 307 | 67.9 | 76.0 | 64.4 | 69.7 | 76.2 | 67.0 |
| 215 | 63.0 | 78.6 | 56.4 | 64.4 | 76.2 | 59.4 |
| 122 | 71.9 | 79.0 | 68.8 | 71.3 | 76.0 | 69.4 |
| 38 | 66.4 | 77.9 | 61.5 | 68.1 | 76.0 | 64.7 |
| 54 | 71.4 | 78.5 | 68.3 | 70.8 | 75.8 | 68.7 |
| 225 | 71.0 | 78.1 | 68.0 | 70.6 | 75.6 | 68.5 |
| 26 | 65.7 | 76.6 | 61.0 | 65.8 | 75.6 | 61.6 |
| 298 | 66.3 | 76.8 | 61.8 | 66.5 | 75.4 | 62.6 |
| 114 | 64.7 | 74.2 | 60.6 | 66.3 | 75.4 | 62.4 |
| 185 | 69.0 | 76.4 | 65.8 | 70.6 | 75.2 | 68.6 |
| 128 | 68.6 | 77.7 | 64.7 | 69.6 | 75.2 | 67.2 |
| 109 | 68.2 | 75.0 | 65.3 | 69.4 | 75.2 | 66.9 |
| 104 | 67.4 | 71.8 | 65.6 | 66.0 | 75.2 | 62.1 |
| 277 | 65.8 | 74.5 | 62.2 | 64.6 | 75.2 | 60.0 |
| 327 | 65.0 | 76.4 | 60.1 | 64.6 | 75.2 | 60.0 |
| 303 | 68.3 | 76.1 | 65.0 | 68.9 | 75.0 | 66.3 |
| 181 | 69.1 | 76.3 | 66.0 | 69.8 | 74.8 | 67.7 |
| 59 | 66.8 | 74.9 | 63.4 | 67.7 | 74.8 | 64.7 |
| 209 | 63.2 | 73.2 | 58.9 | 65.3 | 74.8 | 61.2 |
| 236 | 61.7 | 76.1 | 55.6 | 63.2 | 74.8 | 58.3 |
| 214 | 66.6 | 74.7 | 63.2 | 67.9 | 74.4 | 65.1 |
| 51 | 65.1 | 76.6 | 60.1 | 65.0 | 74.2 | 61.0 |
| 140 | 65.9 | 75.5 | 61.8 | 64.7 | 74.2 | 60.7 |
| 99 | 69.2 | 75.5 | 66.5 | 69.1 | 74.0 | 67.0 |
| 105 | 66.8 | 75.6 | 63.0 | 67.3 | 74.0 | 64.4 |
| 294 | 63.2 | 74.6 | 58.4 | 64.4 | 74.0 | 60.4 |
| 58 | 59.4 | 75.0 | 52.8 | 59.7 | 73.8 | 53.7 |
| 143 | 70.1 | 75.9 | 67.6 | 71.2 | 73.6 | 70.2 |
| 272 | 65.6 | 78.1 | 60.2 | 65.8 | 73.6 | 62.5 |
| 101 | 65.1 | 74.5 | 61.1 | 65.7 | 73.4 | 62.4 |
| 144 | 68.3 | 75.4 | 65.3 | 68.2 | 73.0 | 66.1 |
| 42 | 68.3 | 77.5 | 64.4 | 67.6 | 73.0 | 65.3 |
| 180 | 68.3 | 76.6 | 64.8 | 67.7 | 72.8 | 65.5 |
| 170 | 72.0 | 74.7 | 70.9 | 72.7 | 72.6 | 72.7 |
| 47 | 68.5 | 71.0 | 67.4 | 69.7 | 72.6 | 68.4 |
| 44 | 68.7 | 77.6 | 64.9 | 68.5 | 72.6 | 66.8 |
| 16 | 65.2 | 71.8 | 62.4 | 67.8 | 72.4 | 65.8 |
| 124 | 65.1 | 74.7 | 61.0 | 66.5 | 72.4 | 63.9 |
| 241 | 64.8 | 72.1 | 61.7 | 66.0 | 72.4 | 63.3 |
| 46 | 68.2 | 73.0 | 66.2 | 67.4 | 72.2 | 65.3 |
| 321 | 65.7 | 75.6 | 61.4 | 66.5 | 72.2 | 64.1 |
| 130 | 69.1 | 72.4 | 67.7 | 69.9 | 72.0 | 68.9 |
| 79 | 64.3 | 71.8 | 61.1 | 66.3 | 72.0 | 63.8 |
| 247 | 62.1 | 71.2 | 58.2 | 64.4 | 72.0 | 61.2 |
| 262 | 65.3 | 70.0 | 63.2 | 68.0 | 71.7 | 66.4 |
| 95 | 66.6 | 72.4 | 64.1 | 67.0 | 71.7 | 65.0 |
| 142 | 65.5 | 71.2 | 63.0 | 65.0 | 71.7 | 62.1 |
| 267 | 64.0 | 73.4 | 60.0 | 64.3 | 71.7 | 61.2 |
| 69 | 67.8 | 71.4 | 66.2 | 68.9 | 71.5 | 67.8 |
| 259 | 62.7 | 69.6 | 59.8 | 65.5 | 71.5 | 63.0 |
| 118 | 65.4 | 72.6 | 62.3 | 67.2 | 71.3 | 65.4 |
| 234 | 65.5 | 71.1 | 63.1 | 65.0 | 71.3 | 62.3 |
| 138 | 65.3 | 71.8 | 62.5 | 64.0 | 71.3 | 60.9 |
| 286 | 69.0 | 68.8 | 69.1 | 69.7 | 71.1 | 69.1 |
| 110 | 67.2 | 68.0 | 66.9 | 69.1 | 71.1 | 68.3 |
| 173 | 70.2 | 78.0 | 66.9 | 68.6 | 71.1 | 67.6 |
| 320 | 65.1 | 69.9 | 63.1 | 65.9 | 71.1 | 63.6 |
| 200 | 64.6 | 72.3 | 61.3 | 64.9 | 71.1 | 62.2 |
| 257 | 67.4 | 69.6 | 66.5 | 69.6 | 70.9 | 69.1 |
| 167 | 67.1 | 72.7 | 64.7 | 66.9 | 70.9 | 65.2 |
| 314 | 68.0 | 74.3 | 65.3 | 65.4 | 70.9 | 63.1 |
| 8 | 62.7 | 76.5 | 56.8 | 60.0 | 70.9 | 55.3 |
| 111 | 78.3 | 74.3 | 80.0 | 76.2 | 70.7 | 78.5 |
| 27 | 68.2 | 72.8 | 66.2 | 69.2 | 70.5 | 68.7 |
| 64 | 68.0 | 75.0 | 65.0 | 68.5 | 70.1 | 67.8 |
| 304 | 64.1 | 69.5 | 61.9 | 65.1 | 70.1 | 63.0 |
| 177 | 59.3 | 67.5 | 55.9 | 63.5 | 70.1 | 60.7 |
| 74 | 68.2 | 67.5 | 68.6 | 70.9 | 69.7 | 71.4 |
| 34 | 66.3 | 69.8 | 64.8 | 66.9 | 69.7 | 65.7 |
| 17 | 66.3 | 72.9 | 63.4 | 65.1 | 69.7 | 63.1 |
| 36 | 64.3 | 73.6 | 60.4 | 64.4 | 69.7 | 62.1 |
| 171 | 72.0 | 73.4 | 71.3 | 72.3 | 69.5 | 73.6 |
| 251 | 62.8 | 70.9 | 59.3 | 64.0 | 69.5 | 61.7 |
| 211 | 65.2 | 71.9 | 62.3 | 65.5 | 69.3 | 63.9 |
| 193 | 69.5 | 71.7 | 68.6 | 69.7 | 69.1 | 69.9 |
| 256 | 62.4 | 69.0 | 59.6 | 65.2 | 69.1 | 63.6 |

TABLE 4

| SEQ ID NO: | Coefficient | Constant term |
|---|---|---|
| 188 | 0.46 | −3.96 |
| 164 | 1.52 | −12.87 |
| 85 | 0.76 | −9.89 |
| 13 | 0.44 | −3.44 |
| 175 | −2.56 | 28.63 |
| 137 | 1.19 | −9.35 |
| 231 | 0.52 | −3.92 |
| 319 | 0.50 | −5.37 |
| 195 | 0.52 | −2.98 |
| 263 | 0.74 | −5.56 |
| 165 | 1.72 | −13.28 |
| 226 | 0.90 | −9.97 |
| 94 | 1.04 | −6.31 |
| 45 | 1.09 | −7.88 |
| 190 | 0.52 | −2.78 |
| 274 | −3.35 | 34.66 |
| 328 | 0.90 | −8.02 |
| 80 | 1.00 | −7.29 |
| 220 | 1.16 | −8.43 |
| 198 | 0.71 | −4.51 |
| 98 | 0.90 | −4.61 |
| 43 | 1.05 | −7.31 |
| 316 | 1.29 | −8.88 |
| 2 | 1.01 | −6.10 |
| 115 | 2.20 | −20.21 |
| 299 | 1.00 | −9.37 |
| 50 | 0.47 | −2.72 |
| 150 | 0.56 | −3.02 |
| 196 | 0.56 | −3.19 |
| 31 | 0.53 | −3.07 |
| 182 | 1.11 | −10.40 |
| 72 | 0.75 | −4.45 |
| 318 | 0.74 | −5.73 |
| 149 | 0.81 | −5.37 |
| 312 | 0.61 | −2.91 |
| 96 | 0.70 | −3.37 |
| 329 | 0.81 | −5.51 |
| 70 | 0.53 | −4.19 |
| 40 | 0.86 | −4.96 |
| 127 | 1.53 | −12.09 |
| 153 | 0.76 | −4.05 |
| 183 | −3.32 | 35.60 |
| 148 | 1.25 | −8.23 |
| 68 | 0.68 | −5.43 |
| 3 | 1.39 | −9.03 |
| 60 | 0.86 | −4.09 |
| 66 | 1.97 | −16.60 |
| 25 | 0.56 | −3.00 |
| 75 | 0.61 | −3.65 |
| 12 | 0.98 | −7.25 |
| 255 | 0.72 | −7.17 |
| 7 | 2.37 | −19.98 |
| 1 | 2.02 | −19.02 |
| 291 | 1.25 | −8.99 |
| 162 | 0.63 | −3.74 |
| 163 | 0.81 | −4.70 |
| 87 | 0.73 | −3.97 |
| 199 | 0.90 | −6.60 |
| 120 | 1.00 | −6.11 |
| 222 | 3.07 | −26.17 |
| 311 | 0.59 | −3.50 |
| 278 | −1.45 | 16.91 |
| 260 | 0.90 | −4.36 |
| 246 | 1.85 | −16.38 |
| 197 | 0.57 | −3.42 |
| 103 | 2.01 | −17.60 |
| 22 | 0.53 | −4.56 |
| 106 | 1.05 | −6.86 |
| 322 | 0.95 | −7.58 |
| 57 | 0.56 | −3.59 |
| 309 | 0.71 | −5.45 |
| 29 | 1.18 | −6.74 |
| 184 | −3.26 | 31.56 |
| 206 | 0.66 | −4.32 |
| 135 | 0.89 | −4.22 |
| 179 | 1.56 | −11.11 |
| 287 | 1.88 | −15.11 |
| 56 | 1.31 | −8.81 |
| 207 | 0.63 | −3.95 |
| 261 | 1.04 | −12.53 |
| 201 | 1.27 | −10.31 |
| 217 | 1.58 | −12.07 |
| 317 | 2.32 | −21.17 |
| 172 | 0.85 | −5.07 |
| 300 | 0.91 | −9.12 |
| 102 | 1.40 | −9.99 |
| 285 | 0.94 | −5.62 |
| 20 | 1.03 | −6.33 |
| 159 | 1.19 | −7.36 |
| 21 | 1.16 | −8.70 |
| 73 | −0.53 | 4.41 |
| 78 | 0.48 | −2.44 |
| 15 | 0.87 | −5.39 |
| 30 | 0.43 | −3.27 |
| 134 | 1.00 | −6.04 |
| 315 | 0.74 | −9.76 |
| 76 | 0.75 | −4.45 |
| 107 | 1.18 | −8.80 |
| 97 | 0.57 | −3.71 |
| 23 | 0.91 | −4.91 |
| 33 | 0.86 | −4.72 |
| 307 | 1.66 | −11.62 |
| 215 | −1.63 | 18.47 |
| 122 | 0.80 | −4.37 |
| 38 | 0.83 | −5.46 |
| 54 | 0.67 | −3.37 |
| 225 | 0.63 | −5.27 |
| 26 | 0.52 | −3.11 |
| 298 | 0.87 | −6.15 |
| 114 | 0.64 | −3.71 |
| 185 | 0.81 | −6.70 |
| 128 | 1.01 | −6.33 |
| 109 | −2.71 | 33.33 |
| 104 | 1.28 | −10.29 |
| 277 | 0.63 | −3.80 |
| 327 | 0.52 | −3.47 |
| 303 | 0.78 | −4.47 |
| 181 | 0.87 | −5.65 |
| 59 | 0.67 | −3.76 |
| 209 | 1.09 | −14.30 |
| 236 | 0.88 | −7.04 |
| 214 | 1.28 | −9.55 |
| 51 | 0.64 | −3.99 |
| 140 | 0.81 | −5.16 |
| 99 | 0.73 | −3.95 |
| 105 | 0.77 | −4.44 |
| 294 | 0.62 | −3.83 |
| 58 | −1.09 | 10.55 |
| 143 | 0.62 | −3.40 |
| 272 | 0.96 | −7.96 |
| 101 | 0.93 | −5.82 |
| 144 | 0.67 | −3.81 |
| 42 | −1.55 | 15.71 |
| 180 | 1.31 | −9.04 |
| 170 | 0.57 | −2.49 |
| 47 | 1.03 | −7.03 |
| 44 | 0.64 | −3.54 |
| 16 | 1.18 | −13.90 |
| 124 | 0.97 | −7.04 |
| 241 | −2.20 | 24.78 |
| 46 | 1.29 | −9.94 |
| 321 | 1.00 | −7.64 |
| 130 | 1.67 | −13.02 |
| 79 | 0.65 | −3.61 |
| 247 | 0.69 | −4.92 |
| 262 | 0.84 | −7.44 |
| 95 | 1.45 | −10.46 |
| 142 | 0.65 | −3.63 |
| 267 | 0.69 | −4.79 |
| 69 | 0.55 | −4.42 |
| 259 | 0.64 | −6.21 |
| 118 | −1.78 | 19.05 |
| 234 | 0.72 | −4.36 |
| 138 | 0.57 | −3.21 |
| 286 | −1.05 | 8.89 |
| 110 | 1.63 | −16.52 |
| 173 | 1.60 | −11.35 |
| 320 | 0.62 | −3.53 |
| 200 | 1.40 | −10.95 |
| 257 | 0.51 | −3.87 |
| 167 | 1.29 | −11.15 |
| 314 | 1.59 | −12.95 |
| 8 | 0.66 | −4.07 |
| 111 | 2.44 | −20.94 |
| 27 | 0.38 | −2.72 |
| 64 | 0.45 | −2.24 |
| 304 | −0.61 | 4.99 |
| 177 | 0.59 | −7.14 |
| 74 | 0.86 | −10.97 |
| 34 | 0.84 | −6.99 |
| 17 | 0.77 | −4.63 |
| 36 | 0.69 | −4.14 |
| 171 | 0.57 | −2.50 |
| 251 | 0.85 | −9.59 |
| 211 | 1.02 | −7.34 |
| 193 | 1.57 | −12.18 |
| 256 | 0.47 | −3.00 |

TABLE 5

| | Training cohort | | | Validation cohort | | |
|---|---|---|---|---|---|---|
| | Accu- | Sensi- | Speci- | Accu- | Sensi- | Speci- |
| Gene | racy | tivity | ficity | racy | tivity | ficity |
| 18_164 | 86.4 | 98.0 | 81.4 | 86.7 | 98.2 | 81.8 |
| 255_164 | 86.9 | 96.1 | 83.0 | 86.6 | 96.1 | 82.5 |
| 177_164 | 86.1 | 94.6 | 82.5 | 86.3 | 94.5 | 82.9 |
| 4_164 | 85.8 | 91.1 | 83.5 | 86.0 | 91.3 | 83.7 |
| 164_9 | 85.4 | 92.0 | 82.6 | 85.9 | 91.5 | 83.5 |
| 300_164 | 85.8 | 96.1 | 81.5 | 85.7 | 96.1 | 81.3 |
| 272_164 | 84.8 | 97.6 | 79.4 | 85.7 | 97.0 | 80.9 |
| 206_164 | 86.0 | 95.3 | 82.0 | 85.6 | 93.9 | 82.0 |
| 12_164 | 85.6 | 96.0 | 81.2 | 85.6 | 95.5 | 81.4 |
| 166_164 | 85.6 | 92.8 | 82.5 | 85.6 | 91.7 | 83.0 |
| 168_164 | 85.3 | 97.6 | 80.1 | 85.6 | 97.4 | 80.6 |
| 6_164 | 85.0 | 97.1 | 79.8 | 85.6 | 97.0 | 80.8 |
| 259_164 | 85.0 | 94.7 | 80.8 | 85.5 | 93.9 | 81.9 |
| 115_164 | 84.8 | 96.4 | 79.8 | 85.5 | 96.1 | 81.0 |
| 164_129 | 84.5 | 95.8 | 79.7 | 85.4 | 95.3 | 81.2 |
| 190_164 | 85.5 | 95.4 | 81.3 | 85.3 | 95.1 | 81.2 |
| 199_164 | 85.4 | 95.9 | 81.0 | 85.3 | 95.9 | 80.8 |
| 66_164 | 85.4 | 96.3 | 80.7 | 85.3 | 96.5 | 80.5 |
| 164_107 | 85.4 | 97.1 | 80.4 | 85.3 | 97.2 | 80.3 |
| 207_164 | 85.3 | 96.8 | 80.4 | 85.3 | 95.3 | 81.0 |
| 263_164 | 85.1 | 97.5 | 79.8 | 85.3 | 97.0 | 80.4 |
| 16_164 | 85.0 | 95.3 | 80.6 | 85.3 | 95.1 | 81.2 |
| 37_164 | 84.9 | 94.9 | 80.6 | 85.3 | 94.1 | 81.5 |
| 38_164 | 84.9 | 96.9 | 79.8 | 85.3 | 96.3 | 80.7 |
| 261_164 | 85.5 | 94.8 | 81.6 | 85.2 | 93.9 | 81.5 |
| 305_164 | 85.2 | 94.7 | 81.2 | 85.2 | 93.7 | 81.6 |
| 55_164 | 85.1 | 94.2 | 81.2 | 85.2 | 93.1 | 81.9 |
| 127_164 | 84.6 | 95.6 | 79.9 | 85.2 | 95.1 | 80.9 |
| 104_164 | 84.2 | 96.2 | 79.1 | 85.2 | 96.1 | 80.5 |
| 31_164 | 85.2 | 95.9 | 80.6 | 85.1 | 94.3 | 81.2 |
| 121_164 | 84.7 | 97.8 | 79.0 | 85.1 | 97.4 | 79.8 |
| 81_164 | 84.6 | 95.4 | 79.9 | 85.1 | 95.5 | 80.7 |
| 295_164 | 84.6 | 93.8 | 80.7 | 85.1 | 93.1 | 81.6 |
| 6_165 | 84.5 | 95.3 | 79.9 | 85.1 | 95.9 | 80.5 |
| 25_164 | 84.5 | 96.1 | 79.5 | 85.1 | 95.5 | 80.6 |
| 83_164 | 84.4 | 97.1 | 79.0 | 85.1 | 96.7 | 80.2 |
| 32_164 | 85.1 | 94.7 | 81.0 | 85.0 | 94.1 | 81.1 |
| 247_164 | 84.5 | 95.8 | 79.7 | 85.0 | 95.1 | 80.7 |
| 26_164 | 84.1 | 96.2 | 78.9 | 85.0 | 96.3 | 80.2 |
| 302_164 | 84.5 | 95.4 | 79.8 | 84.9 | 94.7 | 80.7 |
| 268_164 | 84.5 | 97.3 | 79.0 | 84.9 | 96.9 | 79.8 |
| 90_164 | 84.4 | 95.8 | 79.5 | 84.9 | 95.9 | 80.2 |
| 287_164 | 84.2 | 96.9 | 78.8 | 84.9 | 96.7 | 79.9 |
| 278_164 | 84.9 | 96.0 | 80.1 | 84.8 | 94.7 | 80.5 |
| 5_164 | 84.7 | 93.6 | 80.9 | 84.8 | 93.7 | 81.0 |
| 137_164 | 84.5 | 95.9 | 79.7 | 84.8 | 95.5 | 80.3 |
| 276_164 | 84.5 | 94.9 | 80.0 | 84.8 | 94.9 | 80.5 |
| 164_271 | 84.1 | 96.6 | 78.8 | 84.8 | 95.9 | 80.0 |
| 167_164 | 83.8 | 97.4 | 78.0 | 84.8 | 97.2 | 79.4 |
| 126_164 | 84.7 | 96.8 | 79.5 | 84.7 | 96.1 | 79.8 |
| 211_164 | 84.3 | 96.6 | 79.0 | 84.7 | 96.7 | 79.6 |
| 67_164 | 84.2 | 95.2 | 79.4 | 84.7 | 93.7 | 80.9 |
| 39_164 | 83.9 | 95.9 | 78.8 | 84.7 | 96.1 | 79.8 |
| 186_164 | 85.1 | 95.7 | 80.6 | 84.6 | 94.3 | 80.5 |
| 182_164 | 85.0 | 96.0 | 80.3 | 84.6 | 94.7 | 80.4 |
| 226_164 | 85.0 | 96.0 | 80.3 | 84.6 | 95.1 | 80.1 |
| 27_164 | 84.7 | 96.3 | 79.7 | 84.6 | 95.3 | 80.0 |
| 275_164 | 84.6 | 95.7 | 79.8 | 84.6 | 94.1 | 80.5 |
| 185_164 | 84.3 | 95.8 | 79.3 | 84.6 | 95.1 | 80.2 |
| 92_164 | 84.2 | 93.8 | 80.1 | 84.6 | 93.3 | 80.9 |
| 10_164 | 85.0 | 95.9 | 80.4 | 84.5 | 94.7 | 80.1 |
| 279_164 | 84.7 | 93.3 | 81.1 | 84.5 | 92.7 | 80.9 |
| 256_164 | 84.7 | 96.5 | 79.6 | 84.5 | 96.1 | 79.5 |
| 80_164 | 84.6 | 94.8 | 80.2 | 84.5 | 93.3 | 80.8 |
| 164_195 | 84.6 | 95.6 | 79.9 | 84.5 | 94.9 | 80.1 |
| 243_164 | 84.5 | 96.9 | 79.2 | 84.5 | 96.5 | 79.3 |
| 22_164 | 84.5 | 95.4 | 79.8 | 84.5 | 94.7 | 80.1 |
| 164_97 | 84.4 | 96.4 | 79.2 | 84.5 | 95.9 | 79.6 |
| 34_164 | 84.2 | 97.6 | 78.4 | 84.5 | 96.9 | 79.3 |
| 215_164 | 84.2 | 95.1 | 79.5 | 84.5 | 94.1 | 80.4 |
| 187_164 | 84.0 | 95.1 | 79.3 | 84.5 | 93.7 | 80.6 |
| 164_174 | 84.0 | 96.5 | 78.6 | 84.5 | 96.3 | 79.4 |
| 50_164 | 83.9 | 95.8 | 78.8 | 84.5 | 94.9 | 80.0 |
| 62_164 | 83.8 | 96.9 | 78.2 | 84.5 | 96.7 | 79.3 |
| 75_164 | 83.8 | 96.0 | 78.6 | 84.5 | 95.9 | 79.7 |
| 61_164 | 83.8 | 95.4 | 78.9 | 84.5 | 95.9 | 79.6 |
| 89_164 | 83.7 | 96.6 | 78.2 | 84.5 | 96.1 | 79.6 |
| 306_164 | 83.6 | 95.5 | 78.5 | 84.5 | 94.1 | 80.4 |
| 164_286 | 83.5 | 97.5 | 77.6 | 84.5 | 96.9 | 79.3 |
| 85_164 | 84.6 | 95.6 | 79.8 | 84.4 | 94.9 | 79.9 |
| 192_164 | 83.9 | 97.0 | 78.3 | 84.4 | 96.1 | 79.4 |
| 223_164 | 85.1 | 95.6 | 80.6 | 84.3 | 94.3 | 80.1 |
| 299_164 | 84.8 | 96.4 | 79.8 | 84.3 | 95.1 | 79.8 |
| 73_164 | 84.5 | 96.0 | 79.7 | 84.3 | 95.1 | 79.7 |
| 241_164 | 84.2 | 96.5 | 78.9 | 84.3 | 95.9 | 79.4 |
| 118_164 | 84.1 | 97.0 | 78.6 | 84.3 | 95.9 | 79.4 |
| 283_164 | 84.1 | 96.8 | 78.7 | 84.3 | 95.1 | 79.8 |
| 164_77 | 84.0 | 96.5 | 78.6 | 84.3 | 95.9 | 79.3 |
| 175_164 | 83.9 | 95.2 | 79.1 | 84.3 | 93.5 | 80.4 |
| 164_65 | 83.8 | 95.9 | 78.6 | 84.3 | 95.3 | 79.6 |
| 7_164 | 83.6 | 96.4 | 78.2 | 84.3 | 95.3 | 79.6 |
| 205_164 | 83.3 | 97.3 | 77.3 | 84.3 | 96.5 | 79.2 |
| 188_164 | 84.2 | 96.5 | 78.9 | 84.2 | 95.1 | 79.6 |
| 100_164 | 84.1 | 94.8 | 79.5 | 84.2 | 94.1 | 79.9 |
| 13_164 | 84.0 | 96.2 | 78.8 | 84.2 | 95.7 | 79.3 |
| 106_164 | 84.0 | 97.3 | 78.4 | 84.2 | 96.3 | 79.1 |
| 53_164 | 84.0 | 95.6 | 79.0 | 84.2 | 93.5 | 80.3 |
| 64_164 | 83.9 | 96.1 | 78.7 | 84.2 | 96.1 | 79.2 |
| 210_164 | 83.9 | 93.6 | 79.7 | 84.2 | 91.3 | 81.2 |
| 290_164 | 83.9 | 95.4 | 78.9 | 84.2 | 94.1 | 79.9 |
| 164_43 | 83.9 | 98.1 | 77.9 | 84.2 | 96.3 | 79.1 |
| 164_44 | 83.9 | 97.0 | 78.3 | 84.2 | 96.3 | 79.0 |
| 30_164 | 83.8 | 95.9 | 78.7 | 84.2 | 95.1 | 79.6 |
| 21_164 | 83.8 | 96.4 | 78.4 | 84.2 | 95.9 | 79.3 |
| 301_164 | 83.8 | 97.6 | 77.9 | 84.2 | 96.3 | 79.1 |
| 24_164 | 83.7 | 97.2 | 77.9 | 84.2 | 96.5 | 78.9 |
| 45_164 | 83.7 | 96.9 | 78.0 | 84.2 | 96.7 | 78.9 |
| 200_164 | 83.7 | 96.0 | 78.5 | 84.2 | 95.1 | 79.6 |
| 214_164 | 83.6 | 96.8 | 78.0 | 84.2 | 95.7 | 79.3 |
| 170_164 | 83.5 | 97.0 | 77.7 | 84.2 | 96.3 | 79.0 |
| 213_164 | 83.5 | 97.0 | 77.7 | 84.2 | 96.1 | 79.1 |
| 184_164 | 83.5 | 97.6 | 77.5 | 84.2 | 97.0 | 78.8 |
| 292_164 | 83.4 | 95.4 | 78.3 | 84.2 | 93.7 | 80.2 |
| 171_164 | 83.3 | 96.5 | 77.7 | 84.2 | 96.5 | 79.0 |
| 172_164 | 83.3 | 97.6 | 77.3 | 84.2 | 97.0 | 78.8 |
| 264_164 | 84.7 | 95.4 | 80.2 | 84.1 | 94.3 | 79.8 |
| 70_164 | 84.2 | 95.9 | 79.2 | 84.1 | 95.1 | 79.4 |
| 269_164 | 84.1 | 95.3 | 79.3 | 84.1 | 94.1 | 79.8 |
| 239_164 | 84.0 | 96.9 | 78.5 | 84.1 | 95.9 | 79.1 |
| 71_164 | 84.0 | 97.0 | 78.4 | 84.1 | 96.7 | 78.8 |
| 234_164 | 83.7 | 97.4 | 77.9 | 84.1 | 96.7 | 78.8 |
| 78_164 | 83.6 | 96.1 | 78.3 | 84.1 | 95.3 | 79.3 |
| 96_164 | 83.6 | 96.3 | 78.1 | 84.1 | 95.7 | 79.2 |
| 19_164 | 83.6 | 97.6 | 77.7 | 84.1 | 96.7 | 78.8 |
| 138_164 | 83.6 | 97.0 | 77.9 | 84.1 | 95.7 | 79.2 |
| 245_164 | 83.3 | 97.0 | 77.5 | 84.1 | 96.3 | 78.9 |
| 111_164 | 84.1 | 94.7 | 79.6 | 84.0 | 94.1 | 79.7 |
| 304_164 | 84.1 | 96.2 | 78.9 | 84.0 | 94.9 | 79.4 |
| 250_164 | 83.9 | 95.0 | 79.2 | 84.0 | 94.9 | 79.4 |
| 178_164 | 83.9 | 96.2 | 78.6 | 84.0 | 94.9 | 79.4 |
| 289_164 | 83.9 | 94.9 | 79.3 | 84.0 | 93.7 | 79.9 |
| 84_164 | 83.9 | 95.9 | 78.8 | 84.0 | 94.9 | 79.3 |
| 116_164 | 83.9 | 95.8 | 78.8 | 84.0 | 94.9 | 79.3 |
| 180_164 | 83.7 | 96.7 | 78.1 | 84.0 | 96.1 | 78.8 |
| 94_164 | 83.7 | 96.5 | 78.2 | 84.0 | 95.1 | 79.3 |
| 164_253 | 83.7 | 96.5 | 78.3 | 84.0 | 95.3 | 79.3 |
| 82_164 | 83.6 | 97.0 | 77.9 | 84.0 | 95.9 | 78.9 |
| 132_164 | 83.6 | 96.7 | 78.0 | 84.0 | 96.1 | 78.8 |
| 265_164 | 83.6 | 94.8 | 78.8 | 84.0 | 93.7 | 79.8 |
| 274_164 | 83.6 | 97.4 | 77.8 | 84.0 | 96.1 | 78.9 |
| 164_252 | 83.6 | 97.0 | 78.0 | 84.0 | 96.1 | 78.9 |
| 41_164 | 83.5 | 96.5 | 78.0 | 84.0 | 95.7 | 79.1 |
| 230_164 | 83.5 | 97.0 | 77.7 | 84.0 | 96.5 | 78.7 |
| 191_164 | 83.5 | 96.1 | 78.1 | 84.0 | 95.1 | 79.3 |
| 196_164 | 83.5 | 96.5 | 77.9 | 84.0 | 96.3 | 78.8 |
| 112_164 | 83.5 | 96.9 | 77.8 | 84.0 | 96.3 | 78.8 |
| 280_164 | 83.4 | 96.6 | 77.7 | 84.0 | 96.3 | 78.8 |
| 99_164 | 83.4 | 97.0 | 77.6 | 84.0 | 96.1 | 78.8 |
| 224_164 | 83.4 | 96.3 | 78.0 | 84.0 | 95.7 | 79.0 |
| 193_164 | 83.4 | 97.5 | 77.4 | 84.0 | 96.5 | 78.7 |
| 17_164 | 83.4 | 97.7 | 77.3 | 84.0 | 96.7 | 78.6 |
| 20_164 | 83.4 | 96.5 | 77.8 | 84.0 | 95.5 | 79.1 |
| 164_108 | 83.4 | 97.1 | 77.5 | 84.0 | 96.1 | 78.8 |
| 28_164 | 83.3 | 96.7 | 77.6 | 84.0 | 95.7 | 79.1 |
| 298_164 | 83.3 | 97.3 | 77.3 | 84.0 | 96.5 | 78.7 |
| 240_164 | 83.3 | 95.3 | 78.2 | 84.0 | 94.9 | 79.4 |
| 198_164 | 83.2 | 97.0 | 77.3 | 84.0 | 96.3 | 78.8 |
| 220_164 | 84.2 | 96.7 | 78.9 | 83.9 | 95.7 | 78.8 |
| 124_164 | 84.1 | 97.4 | 78.4 | 83.9 | 96.1 | 78.8 |
| 236_164 | 84.0 | 94.7 | 79.5 | 83.9 | 93.9 | 79.7 |
| 282_164 | 83.9 | 96.0 | 78.8 | 83.9 | 94.9 | 79.2 |
| 235_164 | 83.9 | 96.6 | 78.4 | 83.9 | 96.1 | 78.8 |
| 232_164 | 83.9 | 95.1 | 79.2 | 83.9 | 93.1 | 80.0 |
| 57_164 | 83.8 | 95.5 | 78.8 | 83.9 | 94.3 | 79.4 |
| 277_164 | 83.8 | 96.8 | 78.2 | 83.9 | 95.5 | 78.9 |
| 173_164 | 83.7 | 96.8 | 78.1 | 83.9 | 95.5 | 78.9 |
| 227_164 | 83.7 | 96.0 | 78.4 | 83.9 | 94.9 | 79.2 |
| 69_164 | 83.7 | 97.0 | 78.1 | 83.9 | 95.9 | 78.8 |
| 47_164 | 83.7 | 97.0 | 78.0 | 83.9 | 95.9 | 78.8 |
| 122_164 | 83.6 | 96.5 | 78.1 | 83.9 | 95.5 | 79.0 |
| 242_164 | 83.6 | 97.2 | 77.8 | 83.9 | 96.1 | 78.8 |
| 176_164 | 83.6 | 97.2 | 77.7 | 83.9 | 96.3 | 78.6 |
| 202_164 | 83.6 | 97.3 | 77.7 | 83.9 | 96.1 | 78.7 |
| 197_164 | 83.6 | 97.1 | 77.8 | 83.9 | 96.3 | 78.7 |
| 218_164 | 83.6 | 97.1 | 77.9 | 83.9 | 96.1 | 78.8 |
| 46_164 | 83.5 | 97.0 | 77.7 | 83.9 | 96.3 | 78.7 |
| 86_164 | 83.5 | 97.4 | 77.6 | 83.9 | 96.5 | 78.5 |
| 93_164 | 83.5 | 97.0 | 77.8 | 83.9 | 96.3 | 78.6 |
| 63_164 | 83.5 | 96.5 | 78.0 | 83.9 | 96.1 | 78.7 |
| 109_164 | 83.5 | 96.9 | 77.9 | 83.9 | 95.7 | 78.9 |
| 204_164 | 83.5 | 96.9 | 77.8 | 83.9 | 95.7 | 78.8 |
| 216_164 | 83.5 | 97.1 | 77.7 | 83.9 | 96.3 | 78.6 |
| 169_164 | 83.5 | 96.7 | 77.9 | 83.9 | 95.7 | 78.8 |
| 164_254 | 83.5 | 96.6 | 77.9 | 83.9 | 95.9 | 78.8 |
| 164_203 | 83.5 | 97.0 | 77.7 | 83.9 | 96.3 | 78.7 |
| 98_164 | 83.4 | 97.0 | 77.7 | 83.9 | 96.1 | 78.7 |
| 221_164 | 83.4 | 97.3 | 77.5 | 83.9 | 96.7 | 78.4 |
| 233_164 | 83.4 | 96.0 | 78.0 | 83.9 | 95.1 | 79.1 |
| 284_164 | 83.4 | 96.9 | 77.6 | 83.9 | 96.5 | 78.6 |
| 42_164 | 83.4 | 97.1 | 77.6 | 83.9 | 96.1 | 78.8 |
| 56_164 | 83.4 | 97.2 | 77.6 | 83.9 | 95.9 | 78.8 |
| 296_164 | 83.4 | 97.1 | 77.5 | 83.9 | 96.3 | 78.6 |
| 36_164 | 83.4 | 97.0 | 77.6 | 83.9 | 96.1 | 78.8 |
| 164_229 | 83.4 | 97.1 | 77.5 | 83.9 | 96.7 | 78.4 |
| 134_164 | 83.3 | 97.0 | 77.4 | 83.9 | 96.7 | 78.4 |
| 294_164 | 83.3 | 97.1 | 77.3 | 83.9 | 96.5 | 78.5 |
| 113_164 | 83.3 | 97.3 | 77.3 | 83.9 | 96.5 | 78.5 |
| 164_273 | 83.3 | 97.6 | 77.2 | 83.9 | 96.5 | 78.6 |
| 164_238 | 83.2 | 97.6 | 77.1 | 83.9 | 97.2 | 78.3 |
| 74_164 | 83.0 | 96.9 | 77.1 | 83.9 | 95.9 | 78.8 |
| 102_164 | 84.5 | 95.9 | 79.6 | 83.8 | 93.5 | 79.7 |
| 270_164 | 84.3 | 95.8 | 79.4 | 83.8 | 93.5 | 79.6 |
| 189_164 | 84.0 | 95.5 | 79.0 | 83.8 | 95.1 | 79.0 |
| 262_164 | 84.0 | 95.5 | 79.0 | 83.8 | 94.5 | 79.2 |
| 164_58 | 83.9 | 94.2 | 79.5 | 83.8 | 92.7 | 79.9 |
| 164_251 | 83.9 | 96.0 | 78.7 | 83.8 | 94.1 | 79.4 |
| 103_164 | 83.8 | 96.5 | 78.3 | 83.8 | 95.7 | 78.8 |
| 208_164 | 83.8 | 97.1 | 78.1 | 83.8 | 96.5 | 78.4 |
| 110_164 | 83.8 | 95.5 | 78.8 | 83.8 | 94.5 | 79.2 |
| 281_164 | 83.7 | 96.0 | 78.4 | 83.8 | 95.3 | 78.9 |
| 68_164 | 83.7 | 96.6 | 78.1 | 83.8 | 95.3 | 78.8 |
| 249_164 | 83.7 | 97.0 | 78.1 | 83.8 | 96.5 | 78.3 |
| 219_164 | 83.6 | 97.2 | 77.9 | 83.8 | 95.7 | 78.8 |
| 120_164 | 83.6 | 96.8 | 77.9 | 83.8 | 95.9 | 78.7 |
| 225_164 | 83.6 | 96.5 | 78.1 | 83.8 | 95.3 | 78.9 |
| 52_164 | 83.6 | 97.0 | 77.9 | 83.8 | 96.1 | 78.6 |
| 297_164 | 83.6 | 97.0 | 77.9 | 83.8 | 95.7 | 78.7 |
| 212_164 | 83.6 | 97.0 | 77.8 | 83.8 | 95.9 | 78.6 |
| 248_164 | 83.6 | 97.0 | 77.8 | 83.8 | 95.9 | 78.6 |
| 88_164 | 83.6 | 97.3 | 77.8 | 83.8 | 96.1 | 78.6 |
| 257_164 | 83.6 | 96.8 | 78.0 | 83.8 | 96.1 | 78.5 |
| 244_164 | 83.6 | 97.4 | 77.7 | 83.8 | 96.1 | 78.6 |
| 258_164 | 83.6 | 97.0 | 77.9 | 83.8 | 95.9 | 78.7 |
| 164_79 | 83.6 | 97.3 | 77.7 | 83.8 | 96.3 | 78.5 |
| 181_164 | 83.5 | 97.0 | 77.7 | 83.8 | 96.1 | 78.6 |
| 117_164 | 83.5 | 97.0 | 77.7 | 83.8 | 96.1 | 78.5 |
| 51_164 | 83.5 | 96.7 | 77.9 | 83.8 | 96.1 | 78.5 |
| 209_164 | 83.5 | 97.0 | 77.7 | 83.8 | 95.9 | 78.6 |
| 72_164 | 83.5 | 96.8 | 77.9 | 83.8 | 96.1 | 78.6 |
| 101_164 | 83.5 | 96.8 | 77.9 | 83.8 | 95.7 | 78.7 |
| 59_164 | 83.5 | 97.0 | 77.7 | 83.8 | 96.1 | 78.5 |
| 222_164 | 83.5 | 96.5 | 77.9 | 83.8 | 95.9 | 78.6 |
| 123_164 | 83.5 | 96.8 | 77.9 | 83.8 | 95.7 | 78.7 |
| 266_164 | 83.5 | 96.9 | 77.8 | 83.8 | 95.9 | 78.7 |
| 133_164 | 83.5 | 97.0 | 77.7 | 83.8 | 95.9 | 78.6 |
| 91_164 | 83.5 | 96.7 | 77.9 | 83.8 | 96.1 | 78.6 |
| 35_164 | 83.5 | 97.1 | 77.6 | 83.8 | 96.3 | 78.5 |
| 54_164 | 83.5 | 97.1 | 77.7 | 83.8 | 96.3 | 78.5 |
| 293_164 | 83.5 | 96.9 | 77.9 | 83.8 | 95.9 | 78.7 |
| 291_164 | 83.5 | 97.4 | 77.6 | 83.8 | 96.1 | 78.5 |
| 164_267 | 83.5 | 97.0 | 77.8 | 83.8 | 96.1 | 78.6 |
| 164_285 | 83.5 | 96.4 | 78.0 | 83.8 | 95.9 | 78.6 |
| 164 | 83.4 | 97.0 | 77.7 | 83.8 | 95.9 | 78.6 |
| 167_1 | 81.9 | 89.1 | 78.8 | 82.6 | 86.0 | 81.1 |
| 166_217 | 82.3 | 82.4 | 82.3 | 82.3 | 80.7 | 83.0 |
| 183_169 | 81.9 | 89.3 | 78.7 | 82.3 | 89.0 | 79.4 |
| 246_5 | 81.0 | 86.8 | 78.6 | 82.2 | 87.6 | 79.9 |
| 3_168 | 81.3 | 89.0 | 78.1 | 82.0 | 86.8 | 80.0 |
| 165_2 | 81.2 | 90.1 | 77.4 | 81.8 | 90.2 | 78.3 |
| 166_87 | 82.9 | 88.5 | 80.5 | 81.5 | 86.6 | 79.3 |
| 60_166 | 81.3 | 84.3 | 80.0 | 81.5 | 83.9 | 80.5 |
| 165_136 | 80.0 | 94.4 | 73.9 | 81.5 | 94.9 | 75.7 |
| 7_29 | 80.5 | 84.8 | 78.7 | 81.3 | 84.1 | 80.1 |
| 49_165 | 80.4 | 93.5 | 74.8 | 81.3 | 93.9 | 75.9 |
| 231_165 | 80.2 | 92.7 | 74.8 | 81.2 | 92.9 | 76.2 |
| 165_237 | 80.1 | 90.4 | 75.7 | 81.2 | 92.3 | 76.5 |
| 2_260 | 81.0 | 88.6 | 77.7 | 81.0 | 87.4 | 78.3 |
| 165_288 | 80.4 | 93.6 | 74.8 | 81.0 | 94.7 | 75.1 |
| 165_131 | 80.3 | 95.7 | 73.7 | 81.0 | 96.1 | 74.6 |
| 303_1 | 80.2 | 86.7 | 77.5 | 80.8 | 84.4 | 79.3 |
| 1_15 | 80.0 | 86.2 | 77.4 | 80.7 | 83.3 | 79.6 |
| 119_2 | 80.8 | 88.6 | 77.4 | 80.6 | 86.8 | 77.9 |
| 135_167 | 80.6 | 85.9 | 78.3 | 80.6 | 85.2 | 78.7 |
| 3_228 | 80.1 | 83.0 | 78.8 | 80.6 | 81.9 | 80.1 |
| 3_130 | 80.7 | 85.0 | 78.8 | 80.5 | 83.3 | 79.3 |
| 2_128 | 80.6 | 87.3 | 77.8 | 80.5 | 87.4 | 77.5 |
| 23_2 | 80.7 | 87.9 | 77.6 | 80.4 | 86.2 | 77.9 |
| 33_1 | 80.6 | 85.8 | 78.5 | 80.4 | 83.5 | 79.1 |
| 194_1 | 80.5 | 85.4 | 78.5 | 80.4 | 81.9 | 79.8 |
| 8_1 | 80.9 | 87.4 | 78.1 | 80.3 | 82.5 | 79.4 |
| 201_1 | 80.4 | 86.1 | 78.0 | 80.3 | 83.3 | 79.0 |
| 125_1 | 80.0 | 84.7 | 78.1 | 80.3 | 81.7 | 79.8 |
| 48_1 | 81.2 | 88.3 | 78.2 | 80.1 | 82.9 | 78.9 |
| 3_11 | 80.1 | 85.2 | 77.9 | 80.1 | 82.1 | 79.3 |
| 105_2 | 80.1 | 86.9 | 77.1 | 80.1 | 88.4 | 76.6 |
| 4_179 | 80.8 | 81.7 | 80.4 | 80.0 | 82.3 | 79.1 |
| 14_2 | 80.5 | 87.8 | 77.4 | 80.0 | 87.4 | 76.8 |
| 2_40 | 80.4 | 87.8 | 77.3 | 80.0 | 87.2 | 77.0 |
| 95_2 | 80.1 | 87.9 | 76.8 | 80.0 | 86.6 | 77.2 |
| 76_1 | 80.0 | 87.0 | 77.1 | 80.0 | 82.7 | 78.8 |
| 114_1 | 80.0 | 85.9 | 77.4 | 80.0 | 82.7 | 78.9 |

TABLE 6

| SEQ ID NO: | Coefficient 1 | Coefficient 2 | Constant term |
|---|---|---|---|
| 18_164 | −1.60 | 2.07 | −4.82 |
| 255_164 | −0.98 | 2.48 | −11.23 |
| 177_164 | −1.10 | 2.10 | −4.44 |
| 4_164 | −0.85 | 1.78 | −8.30 |
| 164_9 | 1.73 | −0.66 | −9.70 |
| 300_164 | −1.00 | 2.32 | −9.50 |
| 272_164 | −0.85 | 1.91 | −9.04 |
| 206_164 | −0.91 | 2.23 | −12.83 |
| 12_164 | −0.92 | 2.10 | −10.95 |
| 166_164 | −0.55 | 1.58 | −9.95 |
| 168_164 | −1.98 | 1.65 | 3.77 |
| 6_164 | −1.20 | 1.67 | −5.70 |
| 259_164 | −0.82 | 1.95 | −8.42 |
| 115_164 | −1.79 | 2.29 | −2.81 |
| 164_129 | 1.72 | −1.15 | −5.57 |
| 190_164 | −0.48 | 2.13 | −15.45 |
| 199_164 | −1.10 | 2.33 | −11.65 |
| 66_164 | −1.88 | 2.44 | −4.75 |
| 164_107 | 2.03 | −0.94 | −10.10 |
| 207_164 | −0.64 | 2.08 | −13.57 |
| 263_164 | −0.56 | 2.11 | −13.68 |
| 16_164 | −0.88 | 1.86 | −5.46 |
| 37_164 | −1.04 | 1.96 | −9.42 |
| 38_164 | −1.07 | 2.09 | −10.66 |
| 261_164 | −1.03 | 2.07 | −5.02 |
| 305_164 | −1.09 | 1.82 | −5.02 |
| 55_164 | −1.62 | 1.94 | −0.70 |
| 127_164 | −1.50 | 2.16 | −6.33 |
| 104_164 | 0.60 | 1.44 | −17.01 |
| 31_164 | −0.56 | 2.05 | −14.07 |
| 121_164 | −1.11 | 1.74 | −5.58 |
| 81_164 | −0.53 | 1.74 | −11.39 |
| 295_164 | −1.02 | 1.89 | −7.03 |
| 6_165 | −1.66 | 2.22 | −5.42 |
| 25_164 | −0.40 | 1.88 | −13.73 |
| 83_164 | −0.57 | 1.59 | −9.82 |
| 32_164 | −2.50 | 1.64 | 14.46 |
| 247_164 | −0.67 | 1.75 | −9.95 |
| 26_164 | −0.32 | 1.70 | −12.40 |
| 302_164 | −1.77 | 1.59 | 9.32 |
| 268_164 | −0.92 | 1.75 | −7.57 |
| 90_164 | −0.98 | 1.71 | −1.22 |
| 287_164 | −0.85 | 1.89 | −9.10 |
| 278_164 | 1.28 | 2.03 | −32.12 |
| 5_164 | −1.83 | 1.84 | 1.34 |
| 137_164 | −0.56 | 1.98 | −12.35 |
| 276_164 | −1.05 | 1.76 | −3.97 |
| 164_271 | 1.61 | −0.44 | −10.57 |
| 167_164 | 0.63 | 1.44 | −17.66 |
| 126_164 | −1.58 | 1.79 | −1.86 |
| 211_164 | −0.88 | 1.83 | −9.18 |
| 67_164 | −1.06 | 1.72 | −5.62 |
| 39_164 | −1.38 | 1.62 | −2.09 |
| 186_164 | 0.55 | 1.66 | −17.66 |
| 182_164 | −0.93 | 2.15 | −9.42 |
| 226_164 | −0.68 | 2.13 | −10.43 |
| 27_164 | −0.27 | 1.80 | −13.29 |
| 275_164 | −1.38 | 1.65 | −1.88 |
| 185_164 | −0.40 | 1.75 | −11.50 |
| 92_164 | −0.97 | 1.81 | −6.28 |
| 10_164 | −0.86 | 1.79 | −9.84 |
| 279_164 | 0.87 | 1.72 | −21.61 |
| 256_164 | −0.69 | 1.70 | −10.03 |
| 80_164 | −0.65 | 2.02 | −12.33 |
| 164_195 | 1.96 | −0.27 | −15.00 |
| 243_164 | −1.46 | 1.66 | −0.44 |
| 22_164 | −0.39 | 1.97 | −13.31 |
| 164_97 | 1.69 | −0.48 | −11.18 |
| 34_164 | −0.46 | 1.67 | −10.31 |
| 215_164 | 1.34 | 1.94 | −31.62 |
| 187_164 | −0.51 | 1.68 | −8.56 |
| 164_174 | 1.59 | 0.59 | −17.66 |
| 50_164 | −0.24 | 1.85 | −14.26 |
| 62_164 | −0.22 | 1.65 | −12.75 |
| 75_164 | −0.23 | 1.77 | −13.59 |
| 61_164 | 0.79 | 1.52 | −18.90 |
| 89_164 | −0.43 | 1.57 | −7.48 |
| 306_164 | −0.59 | 1.72 | −10.12 |
| 164_286 | 1.45 | −0.32 | −9.57 |
| 85_164 | −0.39 | 1.98 | −11.58 |
| 192_164 | 0.78 | 1.60 | −20.45 |
| 223_164 | −1.58 | 1.66 | 0.62 |
| 299_164 | −0.75 | 2.07 | −10.44 |
| 73_164 | 0.45 | 1.81 | −19.02 |
| 241_164 | −1.09 | 1.46 | −0.04 |
| 118_164 | 0.71 | 1.67 | −21.70 |
| 283_164 | 0.29 | 1.54 | −14.75 |
| 164_77 | 1.74 | −1.41 | −2.70 |
| 175_164 | 1.39 | 1.98 | −32.32 |
| 164_65 | 1.68 | −0.74 | −7.18 |
| 7_164 | 0.43 | 1.43 | −15.72 |
| 205_164 | −0.92 | 1.57 | −3.72 |
| 188_164 | −0.14 | 1.73 | −13.43 |
| 100_164 | −1.13 | 1.66 | −2.07 |
| 13_164 | −0.12 | 1.76 | −13.99 |
| 106_164 | −0.71 | 1.92 | −11.57 |
| 53_164 | −1.10 | 1.61 | 0.96 |
| 64_164 | −0.15 | 1.69 | −13.55 |
| 210_164 | −1.94 | 1.61 | 8.98 |
| 290_164 | −0.45 | 1.65 | −8.84 |
| 164_43 | 1.88 | −0.56 | −12.03 |
| 164_44 | 1.66 | −0.22 | −12.81 |
| 30_164 | −0.17 | 1.76 | −13.60 |
| 21_164 | −0.44 | 1.74 | −11.46 |
| 301_164 | 0.22 | 1.50 | −14.48 |
| 24_164 | −0.37 | 1.51 | −10.00 |
| 45_164 | −0.46 | 1.84 | −12.20 |
| 200_164 | −0.47 | 1.62 | −10.00 |
| 214_164 | −0.45 | 1.66 | −10.68 |
| 170_164 | −0.15 | 1.72 | −13.87 |
| 213_164 | −0.41 | 1.52 | −9.71 |
| 184_164 | −1.04 | 1.39 | −1.69 |
| 292_164 | −1.29 | 1.60 | −1.13 |
| 171_164 | −0.13 | 1.68 | −13.66 |
| 172_164 | 0.26 | 1.46 | −13.87 |
| 264_164 | 0.70 | 1.63 | −18.75 |
| 70_164 | −0.24 | 1.83 | −13.58 |
| 269_164 | −0.78 | 1.69 | −6.52 |
| 239_164 | −0.35 | 1.61 | −10.55 |
| 71_164 | −0.45 | 1.65 | −10.96 |
| 234_164 | 0.38 | 1.49 | −14.91 |
| 78_164 | −0.19 | 1.76 | −13.93 |
| 96_164 | −0.14 | 1.66 | −13.34 |
| 19_164 | 0.27 | 1.52 | −14.36 |
| 138_164 | 0.11 | 1.51 | −13.33 |
| 245_164 | 0.34 | 1.54 | −15.75 |
| 111_164 | 0.71 | 1.38 | −17.76 |
| 304_164 | 0.35 | 1.67 | −16.93 |
| 250_164 | −1.07 | 1.82 | −5.84 |
| 178_164 | −0.65 | 1.54 | −4.79 |
| 289_164 | 0.36 | 1.54 | −15.62 |
| 84_164 | 0.51 | 1.64 | −17.49 |
| 116_164 | −1.61 | 1.54 | 4.66 |
| 180_164 | −0.16 | 1.56 | −12.08 |
| 94_164 | 0.22 | 1.39 | −13.09 |
| 164_253 | 1.50 | −1.23 | 2.24 |
| 82_164 | −0.25 | 1.57 | −11.81 |
| 132_164 | 0.52 | 1.56 | −20.15 |
| 265_164 | −0.76 | 1.67 | −6.92 |
| 274_164 | −0.87 | 1.35 | −2.46 |
| 164_252 | 1.51 | −0.40 | −7.36 |
| 41_164 | −0.28 | 1.55 | −9.93 |
| 230_164 | 0.39 | 1.52 | −17.97 |
| 191_164 | −0.96 | 1.49 | −1.36 |
| 196_164 | −0.13 | 1.70 | −13.61 |
| 112_164 | −0.12 | 1.54 | −12.28 |
| 280_164 | 0.17 | 1.50 | −13.80 |
| 99_164 | 0.05 | 1.51 | −13.01 |
| 224_164 | 0.33 | 1.58 | −15.79 |
| 193_164 | −0.38 | 1.61 | −10.63 |
| 17_164 | 0.27 | 1.48 | −14.10 |
| 20_164 | 0.15 | 1.47 | −13.35 |
| 164_108 | 1.51 | −0.31 | −10.06 |
| 28_164 | −0.59 | 1.54 | −7.68 |
| 298_164 | −0.13 | 1.56 | −12.24 |
| 240_164 | −0.80 | 1.61 | −6.09 |
| 198_164 | −0.14 | 1.65 | −13.04 |
| 220_164 | −0.50 | 1.90 | −12.49 |
| 124_164 | −0.50 | 1.72 | −10.87 |
| 236_164 | −0.51 | 1.71 | −10.38 |
| 282_164 | 0.37 | 1.57 | −15.84 |
| 235_164 | 0.45 | 1.60 | −17.95 |
| 232_164 | −0.54 | 1.69 | −8.55 |
| 57_164 | −0.35 | 1.91 | −13.90 |
| 277_164 | 0.21 | 1.49 | −13.91 |
| 173_164 | −0.09 | 1.55 | −12.45 |
| 227_164 | −0.70 | 1.57 | −5.93 |
| 69_164 | −0.08 | 1.59 | −12.80 |
| 47_164 | −0.15 | 1.58 | −12.28 |
| 122_164 | 0.10 | 1.46 | −12.92 |
| 242_164 | −0.07 | 1.53 | −12.07 |
| 176_164 | −0.29 | 1.55 | −10.76 |
| 202_164 | −0.36 | 1.61 | −10.87 |
| 197_164 | −0.08 | 1.64 | −13.36 |
| 218_164 | −0.51 | 1.52 | −8.70 |
| 46_164 | 0.31 | 1.49 | −14.95 |
| 86_164 | −0.13 | 1.55 | −12.25 |
| 93_164 | −0.13 | 1.52 | −11.99 |
| 63_164 | −0.49 | 1.60 | −9.70 |
| 109_164 | 0.38 | 1.58 | −17.95 |
| 204_164 | −0.20 | 1.51 | −10.38 |
| 216_164 | −0.28 | 1.53 | −10.91 |
| 169_164 | 0.20 | 1.50 | −14.62 |
| 164_254 | 1.53 | −0.24 | −10.19 |
| 164_203 | 1.53 | 0.17 | −14.06 |
| 98_164 | 0.04 | 1.48 | −12.76 |
| 221_164 | −0.15 | 1.56 | −12.18 |
| 233_164 | −0.71 | 1.54 | −5.44 |
| 284_164 | −0.08 | 1.51 | −12.23 |
| 42_164 | −0.10 | 1.50 | −11.66 |
| 56_164 | −0.14 | 1.57 | −12.32 |
| 296_164 | −0.03 | 1.52 | −12.58 |
| 36_164 | −0.02 | 1.53 | −12.80 |
| 164_229 | 1.51 | −0.10 | −12.23 |
| 134_164 | −0.31 | 1.66 | −12.15 |
| 294_164 | 0.11 | 1.51 | −13.40 |
| 113_164 | −0.24 | 1.57 | −11.89 |
| 164_273 | 1.55 | −0.12 | −12.21 |
| 164_238 | 1.60 | −0.63 | −8.26 |
| 74_164 | 0.43 | 1.47 | −17.92 |
| 102_164 | 0.68 | 1.42 | −16.82 |
| 270_164 | 2.03 | 1.83 | −37.88 |
| 189_164 | 0.34 | 1.52 | −15.01 |
| 262_164 | −0.26 | 1.62 | −11.37 |
| 164_58 | 1.71 | 0.83 | −22.48 |
| 164_251 | 1.63 | −0.46 | −8.53 |
| 103_164 | −1.28 | 1.84 | −4.36 |
| 208_164 | 0.68 | 1.60 | −21.40 |
| 110_164 | −0.50 | 1.64 | −8.76 |
| 281_164 | −0.36 | 1.62 | −10.57 |
| 68_164 | −0.12 | 1.66 | −13.02 |
| 249_164 | −0.44 | 1.52 | −10.19 |
| 219_164 | −0.30 | 1.53 | −10.58 |
| 120_164 | 0.14 | 1.46 | −13.23 |
| 225_164 | −0.16 | 1.67 | −12.79 |
| 52_164 | 0.06 | 1.52 | −13.28 |
| 297_164 | 0.05 | 1.53 | −13.21 |
| 212_164 | 0.06 | 1.52 | −13.64 |
| 248_164 | −0.16 | 1.54 | −11.99 |
| 88_164 | 0.27 | 1.52 | −14.70 |
| 257_164 | −0.08 | 1.57 | −12.63 |
| 244_164 | 0.25 | 1.55 | −14.58 |
| 258_164 | −0.30 | 1.50 | −8.85 |
| 164_79 | 1.50 | 0.13 | −13.38 |
| 181_164 | −0.03 | 1.53 | −12.75 |
| 117_164 | 0.04 | 1.52 | −13.21 |
| 51_164 | −0.12 | 1.56 | −12.44 |
| 209_164 | −0.01 | 1.53 | −12.74 |
| 72_164 | −0.07 | 1.59 | −13.05 |
| 101_164 | −0.14 | 1.55 | −12.27 |
| 59_164 | −0.01 | 1.53 | −12.84 |
| 222_164 | −0.23 | 1.56 | −11.22 |
| 123_164 | 0.07 | 1.52 | −13.33 |
| 266_164 | 0.21 | 1.54 | −15.65 |
| 133_164 | 0.01 | 1.52 | −12.92 |
| 91_164 | −0.20 | 1.51 | −11.31 |
| 35_164 | −0.30 | 1.49 | −10.30 |
| 54_164 | −0.11 | 1.61 | −13.01 |
| 293_164 | 0.06 | 1.53 | −13.43 |
| 291_164 | −0.15 | 1.59 | −12.36 |
| 164_267 | 1.61 | −0.32 | −11.40 |
| 164_285 | 1.61 | −0.19 | −12.48 |
| 164 | 1.52 | NA | −12.87 |
| 167_1 | 1.12 | 1.96 | −28.15 |
| 166_217 | −0.69 | 1.95 | −10.62 |
| 183_169 | −4.31 | 3.31 | 14.07 |
| 246_5 | 3.66 | −3.20 | −2.97 |
| 3_168 | 1.58 | −1.82 | 6.03 |
| 165_2 | 1.07 | 0.64 | −12.10 |
| 166_87 | −0.74 | 0.95 | −0.57 |
| 60_166 | 0.88 | −0.48 | −1.19 |
| 165_136 | 1.72 | −0.72 | −8.51 |
| 7_29 | 1.53 | 0.90 | −18.05 |
| 49_165 | −1.32 | 1.80 | −1.73 |
| 231_165 | 0.32 | 1.27 | −12.22 |
| 165_237 | 1.91 | −0.81 | −7.22 |
| 2_260 | 0.72 | 0.50 | −6.78 |
| 165_288 | 2.06 | −1.38 | −3.16 |
| 165_131 | 1.75 | −0.84 | −7.21 |
| 303_1 | 0.47 | 1.93 | −20.83 |
| 1_15 | 1.91 | 0.36 | −20.23 |
| 119_2 | −0.51 | 1.19 | −4.27 |
| 135_167 | 0.83 | 0.98 | −12.50 |
| 3_228 | 1.32 | 0.32 | −10.30 |
| 3_130 | 1.22 | 0.90 | −14.91 |
| 2_128 | 0.95 | 0.77 | −10.55 |
| 23_2 | 0.39 | 0.88 | −7.42 |
| 33_1 | 0.44 | 1.82 | −19.51 |
| 194_1 | 0.96 | 2.15 | −30.54 |
| 8_1 | 0.69 | 2.03 | −23.35 |
| 201_1 | 0.56 | 1.75 | −20.94 |
| 125_1 | 0.73 | 2.11 | −28.03 |
| 48_1 | 0.61 | 2.08 | −23.93 |
| 3_11 | 1.45 | −0.64 | −4.99 |
| 105_2 | 0.51 | 0.97 | −8.76 |
| 4_179 | −0.78 | 2.02 | −8.19 |
| 14_2 | −0.38 | 1.01 | −3.29 |
| 2_40 | 0.74 | 0.42 | −6.86 |
| 95_2 | 0.94 | 0.94 | −12.41 |
| 76_1 | 0.40 | 1.90 | −20.30 |
| 114_1 | 0.42 | 1.97 | −20.95 |

### Example 2

<Discriminant Analysis Using Up to Five miRNAs in Combination>

In this Example, discriminants were prepared using one to five gene markers in the training cohort including the lung cancer patients and the test subjects without lung cancer (Table 11b1), and then, the discriminant performance was evaluated in the validation cohort (Table 11b2). Based on the evaluation, genes used in discriminants with high performance were extracted to obtain gene markers that were able to detect lung cancer.

To be more specific, firstly, the miRNA expression levels of the training cohort and the validation cohort obtained in the preceding Reference Examples were combined and normalized by global normalization. Secondly, in order to acquire diagnostic markers with higher reliability, only 396 genes having the gene expression level of 2⁶ or higher in 50% or more of the samples in either of the positive sample group (lung cancer patients) or the negative sample group (healthy subjects, benign bone and soft tissue tumor patients and benign breast disease patients and patients having a cancer other than lung cancer), were selected as analytes.

Thirdly, combinations of one to five genes of the 396 gene above were subjected to the Fisher's discriminant analysis using the 396 gene expression level measurement values described above to construct discriminants for discriminating the presence or absence of lung cancer. In this relation, discriminants with high discriminant performance were searched for using a modified greedy algorithm. Accuracy, sensitivity, and specificity in the validation cohort were further calculated using the discriminants prepared above, and the discriminant performance was validated using independent samples. As a result, total 750 discriminants including top 150 discriminants having higher performance as to the combinations of one to five genes were obtained. The genes contained in these discriminants were selected as other diagnostic markers for the lung cancer patients and the test subjects without lung cancer. In this way, miR-920, miR-1185-1-3p, miR-4327, miR-5739, miR-1185-2-3p, miR-1238-5p, miR-1246, miR-1470, miR-197-5p, miR-208a-5p, miR-2467-3p, miR-3122, miR-3160-5p, miR-320b, miR-3610, miR-3619-3p, miR-3937, miR-4447, miR-4480, miR-4505, miR-4515, miR-4535, miR-4706, miR-4718, miR-4730, miR-4734, miR-4755-3p, miR-4787-3p, miR-5008-5p, miR-5010-5p, miR-504-3p, miR-5100, miR-557, miR-6510-5p, miR-6511b-5p, miR-6515-3p, miR-6722-5p, miR-6737-5p, miR-6766-5p, miR-6769a-5p, miR-6771-5p, miR-6786-5p, miR-6794-5p, miR-6800-3p, miR-6802-5p, miR-6805-3p, miR-6819-5p, miR-6824-5p, miR-6858-5p, miR-6861-5p, miR-6880-3p, miR-7107-5p, miR-7109-5p, miR-7114-5p, miR-8071, miR-204-3p, miR-3154, miR-3960, miR-4433a-5p, miR-4462, miR-4476, miR-4508, miR-4687-3p, miR-4732-5p, miR-4771, miR-642a-3p, miR-6732-5p, miR-6820-5p, miR-6821-5p, miR-6829-5p, miR-6893-5p, miR-7108-3p, miR-7111-5p, miR-885-3p, miR-1343-3p, miR-6746-5p, miR-422a, miR-4632-5p, miR-6791-5p, miR-1225-3p, miR-1233-5p, miR-1268a, miR-1268b, miR-1273g-3p, miR-1290, miR-150-3p, miR-17-3p, miR-1908-5p, miR-1914-3p, miR-296-3p, miR-3131, miR-3162-5p, miR-3197, miR-320a, miR-342-5p, miR-365a-5p, miR-3679-5p, miR-371a-5p, miR-423-5p, miR-4257, miR-4270, miR-4286, miR-4417, miR-4442, miR-4454, miR-4507, miR-4516, miR-451a, miR-4665-3p, miR-4675, miR-4689, miR-4695-5p, miR-4739, miR-4745-5p, miR-5001-5p, miR-5698, miR-6075, miR-6125, miR-614, miR-615-5p, miR-638, miR-650, miR-6717-5p, miR-6721-5p, miR-6741-5p, miR-6752-5p, miR-6780b-5p, miR-6784-5p, miR-6875-5p, miR-744-5p, miR-760, miR-7977, miR-8059, miR-8063, miR-8072, miR-92a-2-5p, miR-1228-3p, miR-1275, miR-1307-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, and miR-7975, and the relevant polynucleotides consisting of nucleotide sequences of SEQ ID NOs: 2, 4, 5, 6, 9, 12, 13, 17, 18, 19, 22, 23, 27, 31, 33, 34, 39, 47, 50, 55, 57, 59, 66, 70, 73, 74, 78, 80, 81, 82, 83, 85, 88, 93, 94, 95, 99, 102, 106, 107, 108, 109, 111, 114, 115, 117, 121, 123, 126 to 131, 136, 139 to 142, 144, 145, 146, 147, 149 to 152, 155 to 160, 162, 164, 165, 166, 168, 169, 173, 177, 183, 184, 185, 188 to 191, 193, 199, 201, 202, 205, 206, 207, 211, 213, 214, 216, 217, 218, 220, 222, 223, 226, 229, 230, 231, 234, 236, 237, 238, 241, 242, 246, 249, 250, 253, 255, 256, 258, 260, 263, 264, 268, 270, 276, 278, 286, 295, 296, 299 to 302, 304, 307, 308, 309, 312 to 326, and 328, were found. Among them, the genes newly found as the marker for examining the presence or absence of lung cancer are polynucleotides consisting of the nucleotide sequences represented by SEQ ID NOs: 164, 165, 166, 168, 169, 173, 177, 183, 184, 185, 188 to 191, 193, 199, 201, 202, 205, 206, 207, 211, 213, 214, 216, 217, 218, 220, 222, 223, 226, 229, 230, 231, 234, 236, 237, 238, 241, 242, 246, 249, 250, 253, 255, 256, 258, 260, 263, 264, 268, 270, 276, 278, 286, 295, 296, 299 to 302, 304, 307, 308, 309, 312 to 326, and 328.

The sensitivities in the validation cohort determined by the discriminants obtained using any single one of the 88 polynucleotides consisting of the nucleotide sequences represented by SEQ ID NOs: 188, 164, 85, 13, 231, 319, 263, 165, 226, 94, 190, 328, 80, 220, 316, 2, 115, 299, 50, 150, 31, 318, 149, 312, 70, 127, 183, 66, 12, 255, 162, 199, 222, 278, 260, 246, 22, 106, 322, 57, 309, 184, 206, 207, 201, 217, 317, 300, 102, 159, 73, 78, 315, 107, 23, 33, 307, 114, 185, 128, 109, 59, 236, 214, 140, 99, 144, 47, 241, 321, 130, 95, 142, 234, 286, 173, 320, 314, 111, 27, 304, 177, 74, 34, 17, 211, 193, and 256 among the polynucleotides described above are shown in Table 3. Also, discriminant coefficients and constant terms are shown in Table 4. In this context, the general sensitivity of the existing marker CEA has been reported as being 69%. Accordingly, it was demonstrated that the polynucleotides represented by these SEQ ID NOs singly detect lung cancer with sensitivity beyond CEA.

The genes represented by SEQ ID NOs: 2, 4, 5, 6, 9, 12, 13, 17, 18, 19, 22, 23, 27, 31, 33, 34, 39, 47, 50, 55, 57, 59, 66, 70, 73, 74, 78, 80, 81, 82, 83, 85, 88, 93, 94, 95, 99, 102, 106, 107, 108, 109, 111, 114, 115, 117, 121, 123, 126 to 131, 136, 139 to 142, 144, 145, 146, 147, 149 to 152, 155 to 160, 162, 164, 165, 166, 168, 169, 173, 177, 183, 184, 185, 188 to 191, 193, 199, 201, 202, 205, 206, 207, 211, 213, 214, 216, 217, 218, 220, 222, 223, 226, 229, 230, 231, 234, 236, 237, 238, 241, 242, 246, 249, 250, 253, 255, 256, 258, 260, 263, 264, 268, 270, 276, 278, 286, 295, 296, 299 to 302, 304, 307, 308, 309, 312 to 326, and 328 provide excellent lung cancer discriminant performance, when the genes are used not only alone but also in combinations of, for example, 2, 3, 4, 5, 6, 7, 8, 9, 10 or more genes. For example, when a discriminant was prepared using gene expression level of the nucleotide sequence represented by SEQ ID NO: 18 alone, the discrimination accuracy in the validation cohort was 61.6%; however, when a discriminant was prepared using two genes (SEQ ID NOs: 18 and 164) in combination, the discrimination accuracy in the validation cohort was 86.7%, if a discriminant was prepared using three genes (SEQ ID NOs: 18, 164 and 255), the discrimination accuracy in the validation cohort was 88.2%, when a discriminant was prepared using four genes (SEQ ID NOs: 18, 121, 130 and 164), the discrimination accuracy in the validation cohort was 88.6%, and when a discriminant was prepared using five genes (SEQ ID NOs: 18, 121, 130, 136 and 164), the discrimination accuracy in the validation cohort was 88.8%.

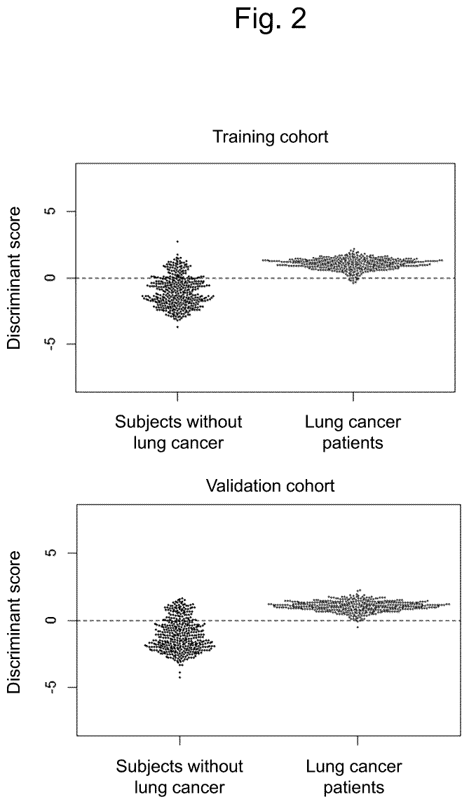

As to the discriminant prepared using measurement values of nucleotide sequences represented by SEQ ID NOs: 164, 18, 268, 147 and 184 in combination, discriminant scores of 1,186 lung cancer patients and 2,777 test subjects without lung cancer in the training cohort were significantly separated, as shown in the upper panel of FIG. 2.

The same results were able to be reproduced also in the validation cohort (FIG. 2, lower panel).

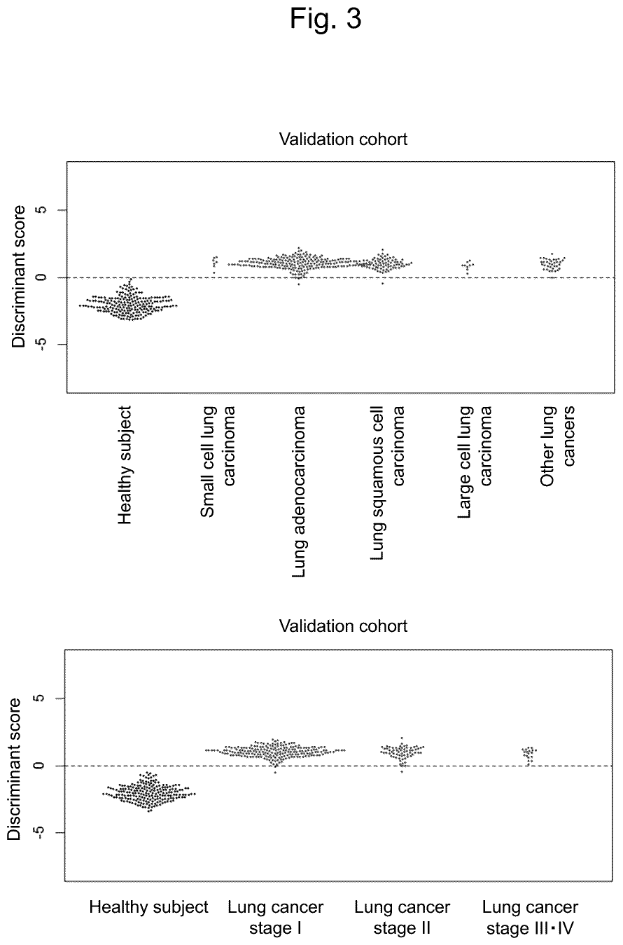

The discriminant scores were categorized on the basis of the histological types and stages of the lung cancer patients. As a result, it was confirmed that lung cancer in all of the categories was able to be detected with high sensitivity (FIG. 3).

Of the 750 discriminants obtained above, the number of discriminants exhibiting a discrimination accuracy of 85% or more both in the training cohort and the validation cohort was 305. These discriminants having particularly high discriminant performance contained at least one of the genes represented by SEQ ID NOs: 18, 4, 130, 2, 9, 17, and 121. These seven genes are referred to as “cancer type-specific polynucleotide group”.

Specifically, when measurement was carried out using a polynucleotide consisting of the nucleotide sequence represented by SEQ ID NO: 18 or a complementary sequence thereof as a target nucleic acid, discrimination accuracy is shown in Table 7-1. The measurement using a combination of 2, 3, 4 or 5 genes comprising the polynucleotide consisting of the nucleotide sequence represented by SEQ ID NO: 18 or a complementary sequence thereof exhibited the highest accuracy of 86.7%, 88.2%, 88.6% and 88.8%, respectively, in the validation cohort.

Further, when measurement was carried out using a polynucleotide consisting of the nucleotide sequence represented by SEQ ID NO: 4 or a complementary sequence thereof as a target nucleic acid, discrimination accuracy is shown in Table 7-2. The measurement using a combination of 2, 3, 4 or 5 genes comprising the polynucleotide consisting of the nucleotide sequence represented by SEQ ID NO: 4 or a complementary sequence thereof exhibited the highest accuracy of 86.0%, 87.1%, 87.8% and 87.8%, respectively, in the validation cohort.

Further, when measurement was carried out using a polynucleotide consisting of the nucleotide sequence represented by SEQ ID NO: 130 or a complementary sequence thereof as a target nucleic acid, discrimination accuracy is shown in Table 7-3. The measurement using a combination of 3, 4 or 5 genes comprising the polynucleotide consisting of the nucleotide sequence represented by SEQ ID NO: 130 or a complementary sequence thereof exhibited the highest accuracy of 86.9%, 88.6% and 88.8%, respectively, in the validation cohort.

Further, when measurement was carried out using a polynucleotide consisting of the nucleotide sequence represented by SEQ ID NO: 2 or a complementary sequence thereof as a target nucleic acid, discrimination accuracy is shown in Table 7-4. The measurement using a combination of 4 or 5 genes comprising the polynucleotide consisting of the nucleotide sequence represented by SEQ ID NO: 2 or a complementary sequence thereof exhibited the highest accuracy of 86.3% and 87.8%, respectively, in the validation cohort.

Further, when measurement was carried out using a polynucleotide consisting of the nucleotide sequence represented by SEQ ID NO: 9 or a complementary sequence thereof as a target nucleic acid, discrimination accuracy is shown in Table 7-5. The measurement using a combination of 3, 4 or 5 genes comprising the polynucleotide consisting of the nucleotide sequence represented by SEQ ID NO: 9 or a complementary sequence thereof exhibited the highest accuracy of 86.9%, 87.2% and 87.6%, respectively, in the validation cohort.

Further, when measurement was carried out using a polynucleotide consisting of the nucleotide sequence represented by SEQ ID NO: 17 or a complementary sequence thereof as a target nucleic acid, discrimination accuracy is shown in Table 7-6. The measurement using a combination of 3, 4 or 5 genes comprising the polynucleotide consisting of the nucleotide sequence represented by SEQ ID NO: 17 or a complementary sequence thereof exhibited the highest accuracy of 85.6%, 87.5% and 87.8%, respectively, in the validation cohort.

Further, when measurement was carried out using a polynucleotide consisting of the nucleotide sequence represented by SEQ ID NO: 121 or a complementary sequence thereof as a target nucleic acid, discrimination accuracy is shown in Table 7-7. The measurement using a combination of 3, 4 or 5 genes comprising the polynucleotide consisting of the nucleotide sequence represented by SEQ ID NO: 121 or a complementary sequence thereof exhibited the highest accuracy of 86.9%, 88.6% and 88.8%, respectively, in the validation cohort.

Further, lung adenocarcinoma, lung squamous cell carcinoma, small cell lung carcinoma and large cell lung carcinoma were able to be detected with average sensitivities of 96.4%, 97.1%, 97.8% and 97.6%, respectively, determined by the 305 discriminants obtained using the polynucleotides consisting of SEQ ID NOs described in Tables 7-1 to 7-7 in combination. In addition, stage I (IA and IIA), stage II (IIA and IIB), and stage III/IV (IIIA, IIIB and IV) of lung cancer were able to be detected with average sensitivities of 96.9%, 94.0% and 94.6%, respectively, determined by the 305 discriminants obtained using the polynucleotides consisting of SEQ ID NOs described in Tables 7-1 to 7-7 in combination. Accordingly, the polynucleotides obtained in this Example exerted a high detection ability without missing a particular histological type or stage of progression of lung cancer. From the above-mentioned results, the polynucleotides consisting of the nucleotide sequences of SEQ ID NOs: 2, 4, 5, 6, 9, 12, 13, 17, 18, 19, 22, 23, 27, 31, 33, 34, 39, 47, 50, 55, 57, 59, 66, 70, 73, 74, 78, 80, 81, 82, 83, 85, 88, 93, 94, 95, 99, 102, 106, 107, 108, 109, 111, 114, 115, 117, 121, 123, 126 to 131, 136, 139 to 142, 144, 145, 146, 147, 149 to 152, 155 to 160, 162, 164, 165, 166, 168, 169, 173, 177, 183, 184, 185, 188 to 191, 193, 199, 201, 202, 205, 206, 207, 211, 213, 214, 216, 217, 218, 220, 222, 223, 226, 229, 230, 231, 234, 236, 237, 238, 241, 242, 246, 249, 250, 253, 255, 256, 258, 260, 263, 264, 268, 270, 276, 278, 286, 295, 296, 299 to 302, 304, 307, 308, 309, 312 to 326, and 328, obtained in this Example, are deemed to be gene groups that lung cancer patients can be specifically discriminated from any of healthy subjects, benign bone and soft tissue tumor patients and benign breast disease patients, and patients having a cancer other than lung cancer. It was further demonstrated that high lung cancer discriminant performance can be obtained by using multiple polynucleotides in combination as target nucleic acids rather than using a single polynucleotide or fewer polynucleotides in combination. In this relation, particularly high discriminant performance can be obtained by using the polynucleotides contained in the cancer type-specific polynucleotide group in combination, wherein the combination of the multiple polynucleotides is not limited to those mentioned above. Even if the polynucleotides are used in any combination, lung cancer can be detected.

Specifically, as shown in the preceding Examples 1 or 2, it is concluded that in all of the polynucleotides consisting of the nucleotide sequences represented by SEQ ID NOs: 1 to 329 or complementary sequences thereof as the target nucleic acids, there exist combinations of 1, 2, 3, 4 or 5 genes that exhibit discriminant performance beyond the existing lung cancer markers, thus indicating that the polynucleotides are excellent diagnostic markers for lung cancer that can detect any histological type or stage of progression of lung cancer described in the preceding Reference Example.

TABLE 7-1

| Combined | | Validation cohort | | |
|---|---|---|---|---|
| gene number | SEQ ID NO: | Sensitivity | Specificity | Accuracy |
| 2 | 18_164 | 98.2 | 81.8 | 86.7 |
| 3 | 18_164_255 | 98.2 | 83.9 | 88.2 |
| 3 | 18_164_300 | 97.8 | 83.0 | 87.5 |
| 3 | 18_164_190 | 97.6 | 83.0 | 87.4 |
| 3 | 18_85_164 | 97.2 | 83.2 | 87.4 |
| 3 | 18_147_164 | 98.2 | 82.5 | 87.2 |
| 3 | 18_22_164 | 97.4 | 82.8 | 87.2 |
| 3 | 18_164_312 | 98.2 | 82.3 | 87.1 |
| 3 | 18_66_164 | 97.8 | 82.5 | 87.1 |
| 3 | 18_78_164 | 97.8 | 82.4 | 87.0 |
| 3 | 18_27_164 | 97.4 | 82.5 | 87.0 |
| 3 | 18_164_207 | 98.2 | 82.0 | 86.9 |
| 3 | 18_82_164 | 98.0 | 82.1 | 86.9 |
| 3 | 18_164_263 | 98.4 | 81.9 | 86.8 |
| 3 | 18_164_168 | 98.2 | 82.0 | 86.8 |
| 3 | 18_34_164 | 98.0 | 82.0 | 86.8 |
| 3 | 18_39_164 | 97.6 | 82.1 | 86.8 |
| 3 | 18_57_164 | 97.6 | 82.2 | 86.8 |
| 3 | 18_121_164 | 98.6 | 81.6 | 86.7 |
| 3 | 18_107_164 | 98.2 | 81.8 | 86.7 |
| 3 | 18_70_164 | 97.6 | 82.0 | 86.7 |
| 3 | 18_50_164 | 97.6 | 82.0 | 86.7 |
| 3 | 18_164_250 | 96.9 | 82.4 | 86.7 |
| 3 | 18_164_315 | 98.2 | 81.6 | 86.6 |
| 3 | 18_164_211 | 98.0 | 81.8 | 86.6 |
| 3 | 18_164_326 | 97.2 | 82.1 | 86.6 |
| 3 | 18_164_308 | 98.4 | 81.3 | 86.4 |
| 3 | 18_164_268 | 98.2 | 81.4 | 86.4 |
| 3 | 18_164_191 | 97.8 | 81.4 | 86.3 |
| 3 | 18_149_165 | 95.5 | 81.2 | 85.5 |
| 4 | 18_121_130_164 | 98.6 | 84.4 | 88.6 |
| 4 | 18_164_255_316 | 98.4 | 84.3 | 88.5 |
| 4 | 18_121_164_255 | 98.0 | 84.2 | 88.4 |
| 4 | 18_147_164_255 | 98.0 | 84.3 | 88.4 |
| 4 | 18_27_164_255 | 97.8 | 84.3 | 88.4 |
| 4 | 18_34_164_255 | 98.0 | 84.1 | 88.3 |
| 4 | 18_47_164_255 | 98.2 | 84.0 | 88.2 |
| 4 | 18_158_164_255 | 98.0 | 84.1 | 88.2 |
| 4 | 18_164_220_255 | 98.0 | 84.0 | 88.2 |
| 4 | 18_88_164_255 | 97.8 | 84.1 | 88.2 |
| 4 | 18_130_164_268 | 98.4 | 83.7 | 88.1 |
| 4 | 18_164_255_321 | 98.2 | 83.8 | 88.1 |
| 4 | 18_164_184_255 | 98.2 | 83.7 | 88.1 |
| 4 | 18_152_164_255 | 98.0 | 83.8 | 88.1 |
| 4 | 18_164_185_255 | 98.0 | 83.9 | 88.1 |
| 4 | 18_164_238_255 | 98.0 | 83.8 | 88.1 |
| 4 | 18_164_255_256 | 97.8 | 83.9 | 88.1 |
| 4 | 18_127_164_255 | 97.6 | 84.1 | 88.1 |
| 4 | 18_164_222_255 | 98.2 | 83.6 | 88.0 |
| 4 | 18_139_164_255 | 98.0 | 83.7 | 88.0 |
| 4 | 18_39_164_255 | 97.6 | 83.9 | 88.0 |
| 4 | 18_164_255_295 | 96.9 | 84.2 | 88.0 |
| 4 | 18_146_164_255 | 97.6 | 83.7 | 87.9 |
| 4 | 18_164_211_255 | 97.4 | 83.7 | 87.8 |
| 4 | 18_164_255_322 | 97.4 | 83.7 | 87.8 |
| 4 | 18_164_255_318 | 98.0 | 83.3 | 87.7 |
| 4 | 18_121_164_201 | 98.2 | 83.0 | 87.6 |
| 4 | 18_147_164_300 | 98.2 | 83.1 | 87.6 |
| 4 | 18_121_151_164 | 98.2 | 83.1 | 87.6 |
| 4 | 18_164_211_300 | 97.2 | 83.0 | 87.3 |
| 4 | 18_95_164_268 | 98.8 | 82.0 | 87.1 |
| 4 | 18_164_231_268 | 98.4 | 82.2 | 87.1 |
| 4 | 18_147_164_268 | 98.4 | 82.2 | 87.1 |
| 4 | 18_164_188_268 | 98.2 | 82.4 | 87.1 |
| 4 | 18_164_268_312 | 98.0 | 82.5 | 87.1 |
| 4 | 18_39_164_300 | 97.4 | 82.6 | 87.1 |
| 4 | 18_95_121_164 | 99.0 | 81.5 | 86.8 |
| 4 | 18_93_164_268 | 98.6 | 81.7 | 86.8 |
| 4 | 18_164_268_308 | 98.6 | 81.6 | 86.7 |
| 4 | 18_107_121_164 | 98.6 | 81.6 | 86.7 |
| 4 | 18_164_218_268 | 98.4 | 81.5 | 86.6 |
| 4 | 18_164_202_268 | 98.2 | 81.6 | 86.6 |
| 4 | 13_18_130_165 | 98.2 | 81.5 | 86.5 |
| 4 | 18_149_165_168 | 96.7 | 82.1 | 86.5 |
| 4 | 18_164_242_268 | 98.2 | 81.5 | 86.5 |
| 4 | 18_164_214_268 | 98.2 | 81.5 | 86.5 |
| 4 | 18_164_268_313 | 98.2 | 81.4 | 86.5 |
| 4 | 18_162_164_268 | 98.0 | 81.5 | 86.5 |
| 4 | 18_150_164_268 | 98.0 | 81.6 | 86.5 |
| 4 | 18_164_268_315 | 98.6 | 81.2 | 86.4 |
| 4 | 18_152_164_268 | 98.6 | 81.2 | 86.4 |
| 4 | 18_164_268_325 | 98.2 | 81.4 | 86.4 |
| 4 | 18_121_149_165 | 97.1 | 81.7 | 86.3 |
| 4 | 13_18_165_260 | 98.6 | 80.8 | 86.1 |
| 4 | 13_18_165_268 | 98.8 | 80.6 | 86.1 |
| 4 | 13_18_121_165 | 99.2 | 80.4 | 86.1 |
| 4 | 13_18_165_168 | 98.2 | 80.9 | 86.1 |
| 4 | 18_149_165_268 | 96.1 | 81.7 | 86.0 |
| 4 | 13_18_83_165 | 98.0 | 80.9 | 86.0 |
| 4 | 13_18_165_263 | 98.6 | 80.5 | 85.9 |
| 4 | 2_18_165_268 | 95.5 | 81.7 | 85.8 |
| 4 | 13_18_165_211 | 98.2 | 80.4 | 85.8 |
| 4 | 13_18_165_256 | 98.2 | 80.4 | 85.7 |
| 4 | 13_18_165_276 | 98.0 | 80.2 | 85.5 |
| 4 | 13_18_165_302 | 98.0 | 80.1 | 85.5 |
| 4 | 13_18_165_190 | 98.2 | 79.9 | 85.3 |
| 5 | 18_121_130_136_164 | 98.6 | 84.6 | 88.8 |
| 5 | 18_121_130_164_314 | 98.4 | 84.6 | 88.8 |
| 5 | 18_114_121_130_164 | 99.0 | 84.3 | 88.7 |
| 5 | 18_121_130_164_214 | 98.6 | 84.5 | 88.7 |
| 5 | 18_121_130_164_193 | 98.8 | 84.3 | 88.6 |
| 5 | 18_130_164_255_268 | 98.6 | 84.4 | 88.6 |
| 5 | 18_121_130_164_320 | 98.6 | 84.4 | 88.6 |
| 5 | 18_121_130_164_301 | 98.6 | 84.3 | 88.6 |
| 5 | 18_121_130_144_164 | 98.6 | 84.4 | 88.6 |
| 5 | 18_121_130_164_168 | 98.4 | 84.5 | 88.6 |
| 5 | 18_121_130_164_205 | 98.4 | 84.4 | 88.6 |
| 5 | 18_121_130_158_164 | 98.8 | 84.1 | 88.5 |
| 5 | 18_121_130_164_260 | 98.8 | 84.1 | 88.5 |
| 5 | 18_106_121_130_164 | 98.8 | 84.1 | 88.5 |
| 5 | 18_121_130_164_318 | 98.6 | 84.2 | 88.5 |
| 5 | 18_121_130_164_286 | 98.6 | 84.1 | 88.5 |
| 5 | 18_121_130_164_315 | 98.6 | 84.2 | 88.5 |
| 5 | 18_121_130_164_237 | 98.6 | 84.2 | 88.5 |
| 5 | 18_121_130_164_184 | 98.6 | 84.2 | 88.5 |
| 5 | 18_121_130_164_270 | 98.4 | 84.2 | 88.5 |
| 5 | 18_121_130_164_309 | 98.4 | 84.2 | 88.5 |
| 5 | 18_121_130_164_278 | 98.4 | 84.3 | 88.5 |
| 5 | 18_82_121_130_164 | 98.4 | 84.2 | 88.5 |
| 5 | 18_23_121_130_164 | 98.4 | 84.2 | 88.5 |
| 5 | 18_121_130_164_189 | 98.4 | 84.3 | 88.5 |
| 5 | 18_121_130_152_164 | 98.2 | 84.3 | 88.5 |
| 5 | 18_121_130_164_213 | 98.2 | 84.3 | 88.5 |
| 5 | 18_121_130_164_229 | 98.2 | 84.3 | 88.5 |
| 5 | 18_57_121_130_164 | 98.2 | 84.4 | 88.5 |
| 5 | 18_121_130_142_164 | 98.8 | 83.9 | 88.4 |
| 5 | 18_121_130_155_164 | 98.6 | 84.0 | 88.4 |
| 5 | 18_39_121_130_164 | 98.4 | 84.1 | 88.4 |
| 5 | 18_27_130_164_268 | 98.4 | 84.1 | 88.4 |
| 5 | 18_33_121_130_164 | 98.4 | 84.1 | 88.4 |
| 5 | 18_121_126_130_164 | 98.4 | 84.1 | 88.4 |
| 5 | 18_121_130_164_319 | 98.4 | 84.1 | 88.4 |
| 5 | 18_22_121_130_164 | 98.2 | 84.1 | 88.4 |
| 5 | 18_59_121_130_164 | 98.2 | 84.1 | 88.4 |
| 5 | 18_27_121_130_164 | 97.8 | 84.4 | 88.4 |
| 5 | 18_130_164_268_317 | 98.2 | 84.1 | 88.3 |
| 5 | 18_121_130_164_201 | 98.2 | 84.1 | 88.3 |
| 5 | 18_34_164_211_255 | 97.6 | 84.3 | 88.3 |
| 5 | 18_19_121_130_164 | 98.4 | 83.9 | 88.2 |
| 5 | 18_74_130_164_268 | 98.4 | 83.7 | 88.1 |
| 5 | 18_130_164_264_268 | 97.2 | 84.1 | 88.0 |
| 5 | 18_39_164_255_328 | 97.4 | 83.9 | 87.9 |
| 5 | 18_39_164_226_255 | 97.4 | 83.9 | 87.9 |
| 5 | 18_95_121_164_188 | 99.0 | 83.0 | 87.8 |
| 5 | 13_18_121_130_165 | 98.4 | 82.5 | 87.2 |
| 5 | 13_18_130_165_268 | 98.6 | 82.3 | 87.2 |
| 5 | 18_151_164_268_315 | 98.0 | 82.3 | 87.0 |
| 5 | 18_147_164_184_268 | 98.8 | 81.9 | 86.9 |
| 5 | 18_149_165_168_268 | 96.7 | 82.6 | 86.8 |
| 5 | 13_18_165_268_276 | 98.0 | 81.3 | 86.3 |
| 5 | 2_18_165_268_301 | 95.3 | 82.2 | 86.1 |
| 5 | 2_18_165_268_315 | 96.3 | 81.8 | 86.1 |
| 5 | 13_18_165_183_268 | 99.0 | 80.2 | 85.8 |
| 5 | 13_18_165_184_268 | 99.0 | 80.1 | 85.8 |

TABLE 7-2

| Combined | | Validation cohort | | |
|---|---|---|---|---|
| gene number | SEQ ID NO: | Sensitivity | Specificity | Accuracy |
| 2 | 4_164 | 91.3 | 83.7 | 86.0 |
| 3 | 4_165_168 | 95.3 | 83.5 | 87.1 |
| 4 | 4_165_168_246 | 97.4 | 83.7 | 87.8 |
| 4 | 4_128_165_168 | 96.3 | 84.0 | 87.6 |
| 4 | 4_117_165_168 | 96.5 | 83.8 | 87.6 |
| 4 | 4_159_165_168 | 95.5 | 84.1 | 87.5 |
| 4 | 4_165_168_260 | 95.3 | 84.2 | 87.5 |
| 4 | 4_17_165_168 | 95.1 | 84.2 | 87.5 |
| 4 | 4_165_168_173 | 96.3 | 83.6 | 87.4 |
| 4 | 4_80_165_168 | 96.5 | 82.6 | 86.8 |
| 4 | 4_99_165_168 | 94.5 | 83.4 | 86.7 |
| 4 | 2_4_168_246 | 93.3 | 82.8 | 85.9 |
| 4 | 4_17_115_168 | 92.1 | 83.3 | 85.9 |
| 4 | 4_17_115_302 | 89.8 | 84.1 | 85.8 |
| 4 | 4_94_173_183 | 93.9 | 82.2 | 85.7 |
| 4 | 2_4_173_183 | 93.3 | 82.2 | 85.5 |
| 4 | 2_4_115_168 | 90.8 | 83.3 | 85.5 |
| 4 | 4_17_115_184 | 91.5 | 82.7 | 85.3 |
| 5 | 4_17_165_168_173 | 96.5 | 84.1 | 87.8 |
| 5 | 4_17_165_168_223 | 95.7 | 84.5 | 87.8 |
| 5 | 4_128_129_165_168 | 94.5 | 84.9 | 87.8 |
| 5 | 2_4_130_168_246 | 95.9 | 84.3 | 87.8 |
| 5 | 4_17_128_165_168 | 95.5 | 84.4 | 87.7 |
| 5 | 4_17_165_168_169 | 95.3 | 84.5 | 87.7 |
| 5 | 4_17_117_165_168 | 95.3 | 84.4 | 87.6 |
| 5 | 4_17_165_168_323 | 95.3 | 84.3 | 87.6 |
| 5 | 4_17_81_165_168 | 94.7 | 84.6 | 87.6 |
| 5 | 4_17_165_168_253 | 95.1 | 84.4 | 87.6 |
| 5 | 4_17_162_165_168 | 95.5 | 84.2 | 87.6 |
| 5 | 2_4_168_201_246 | 94.5 | 84.7 | 87.6 |
| 5 | 4_17_141_165_168 | 94.7 | 84.5 | 87.5 |
| 5 | 4_17_129_165_168 | 94.3 | 84.6 | 87.5 |
| 5 | 4_17_165_168_258 | 94.7 | 84.4 | 87.5 |
| 5 | 4_17_165_168_190 | 95.7 | 84.0 | 87.5 |
| 5 | 4_17_115_168_177 | 92.7 | 85.3 | 87.5 |
| 5 | 4_17_165_168_191 | 94.9 | 84.2 | 87.4 |
| 5 | 4_17_158_165_168 | 95.3 | 84.1 | 87.4 |
| 5 | 4_17_165_168_184 | 95.7 | 83.9 | 87.4 |
| 5 | 4_17_94_165_168 | 95.3 | 84.1 | 87.4 |
| 5 | 4_17_165_168_296 | 95.5 | 83.9 | 87.4 |
| 5 | 4_17_165_168_307 | 95.1 | 84.1 | 87.4 |
| 5 | 4_17_123_165_168 | 95.5 | 83.9 | 87.4 |
| 5 | 4_17_39_165_168 | 94.9 | 84.1 | 87.4 |
| 5 | 4_17_145_165_168 | 95.3 | 83.9 | 87.3 |
| 5 | 4_17_165_168_286 | 94.9 | 84.1 | 87.3 |
| 5 | 4_17_73_165_168 | 95.3 | 83.8 | 87.2 |
| 5 | 4_17_115_165_168 | 94.9 | 84.0 | 87.2 |
| 5 | 4_17_108_165_168 | 94.7 | 84.1 | 87.2 |
| 5 | 4_17_156_165_168 | 94.9 | 84.0 | 87.2 |
| 5 | 4_17_165_168_249 | 95.5 | 83.6 | 87.2 |
| 5 | 4_17_131_165_168 | 95.3 | 83.7 | 87.2 |
| 5 | 4_17_165_168_304 | 94.9 | 83.9 | 87.2 |
| 5 | 4_17_157_165_168 | 95.1 | 83.8 | 87.2 |
| 5 | 4_17_165_168_318 | 94.9 | 83.8 | 87.1 |
| 5 | 4_17_74_165_168 | 94.3 | 84.1 | 87.1 |
| 5 | 4_17_165_168_216 | 94.9 | 83.6 | 87.0 |
| 5 | 4_17_165_168_309 | 94.5 | 83.8 | 87.0 |
| 5 | 4_17_165_168_236 | 94.7 | 83.7 | 87.0 |
| 5 | 4_17_165_168_324 | 95.3 | 83.5 | 87.0 |
| 5 | 2_4_111_168_173 | 92.7 | 84.5 | 86.9 |
| 5 | 4_17_115_130_168 | 92.7 | 84.3 | 86.8 |
| 5 | 2_4_130_168_173 | 93.7 | 83.9 | 86.8 |
| 5 | 4_17_111_115_168 | 91.9 | 84.6 | 86.8 |
| 5 | 2_4_168_173_201 | 93.5 | 83.7 | 86.6 |
| 5 | 4_17_115_160_168 | 92.1 | 84.3 | 86.6 |
| 5 | 4_17_115_168_246 | 94.1 | 83.5 | 86.6 |
| 5 | 2_4_115_168_173 | 94.3 | 83.3 | 86.6 |
| 5 | 4_17_115_168_201 | 92.3 | 84.1 | 86.5 |
| 5 | 4_17_115_168_217 | 92.9 | 83.8 | 86.5 |
| 5 | 2_4_17_115_168 | 92.5 | 83.6 | 86.3 |
| 5 | 4_17_115_140_168 | 90.9 | 84.1 | 86.1 |
| 5 | 4_17_102_115_168 | 92.1 | 83.3 | 85.9 |

TABLE 7-3

| Combined | | Validation cohort | | |
|---|---|---|---|---|
| gene number | SEQ ID NO: | Sensitivity | Specificity | Accuracy |
| 3 | 121_130_164 | 97.6 | 82.3 | 86.9 |
| 4 | 18_121_130_164 | 98.6 | 84.4 | 88.6 |
| 4 | 18_130_164_268 | 98.4 | 83.7 | 88.1 |
| 4 | 13_18_130_165 | 98.2 | 81.5 | 86.5 |
| 5 | 18_121_130_136_164 | 98.6 | 84.6 | 88.8 |
| 5 | 18_121_130_164_314 | 98.4 | 84.6 | 88.8 |
| 5 | 18_114_121_130_164 | 99.0 | 84.3 | 88.7 |
| 5 | 18_121_130_164_214 | 98.6 | 84.5 | 88.7 |
| 5 | 18_121_130_164_193 | 98.8 | 84.3 | 88.6 |
| 5 | 18_130_164_255_268 | 98.6 | 84.4 | 88.6 |
| 5 | 18_121_130_164_320 | 98.6 | 84.4 | 88.6 |
| 5 | 18_121_130_164_301 | 98.6 | 84.3 | 88.6 |
| 5 | 18_121_130_144_164 | 98.6 | 84.4 | 88.6 |
| 5 | 18_121_130_164_168 | 98.4 | 84.5 | 88.6 |
| 5 | 18_121_130_164_205 | 98.4 | 84.4 | 88.6 |
| 5 | 18_121_130_158_164 | 98.8 | 84.1 | 88.5 |
| 5 | 18_121_130_164_260 | 98.8 | 84.1 | 88.5 |
| 5 | 18_106_121_130_164 | 98.8 | 84.1 | 88.5 |
| 5 | 18_121_130_164_318 | 98.6 | 84.2 | 88.5 |
| 5 | 18_121_130_164_286 | 98.6 | 84.1 | 88.5 |
| 5 | 18_121_130_164_315 | 98.6 | 84.2 | 88.5 |
| 5 | 18_121_130_164_237 | 98.6 | 84.2 | 88.5 |
| 5 | 18_121_130_164_184 | 98.6 | 84.2 | 88.5 |
| 5 | 18_121_130_164_270 | 98.4 | 84.2 | 88.5 |
| 5 | 18_121_130_164_309 | 98.4 | 84.2 | 88.5 |
| 5 | 18_121_130_164_278 | 98.4 | 84.3 | 88.5 |
| 5 | 18_82_121_130_164 | 98.4 | 84.2 | 88.5 |
| 5 | 18_23_121_130_164 | 98.4 | 84.2 | 88.5 |
| 5 | 18_121_130_164_189 | 98.4 | 84.3 | 88.5 |
| 5 | 18_121_130_152_164 | 98.2 | 84.3 | 88.5 |
| 5 | 18_121_130_164_213 | 98.2 | 84.3 | 88.5 |
| 5 | 18_121_130_164_229 | 98.2 | 84.3 | 88.5 |
| 5 | 18_57_121_130_164 | 98.2 | 84.4 | 88.5 |
| 5 | 18_121_130_142_164 | 98.8 | 83.9 | 88.4 |
| 5 | 18_121_130_155_164 | 98.6 | 84.0 | 88.4 |
| 5 | 18_39_121_130_164 | 98.4 | 84.1 | 88.4 |
| 5 | 18_27_130_164_268 | 98.4 | 84.1 | 88.4 |
| 5 | 18_33_121_130_164 | 98.4 | 84.1 | 88.4 |
| 5 | 18_121_126_130_164 | 98.4 | 84.1 | 88.4 |
| 5 | 18_121_130_164_319 | 98.4 | 84.1 | 88.4 |
| 5 | 18_22_121_130_164 | 98.2 | 84.1 | 88.4 |
| 5 | 18_59_121_130_164 | 98.2 | 84.1 | 88.4 |
| 5 | 18_27_121_130_164 | 97.8 | 84.4 | 88.4 |
| 5 | 18_130_164_268_317 | 98.2 | 84.1 | 88.3 |
| 5 | 18_121_130_164_201 | 98.2 | 84.1 | 88.3 |
| 5 | 18_19_121_130_164 | 98.4 | 83.9 | 88.2 |
| 5 | 18_74_130_164_268 | 98.4 | 83.7 | 88.1 |
| 5 | 18_130_164_264_268 | 97.2 | 84.1 | 88.0 |
| 5 | 2_4_130_168_246 | 95.9 | 84.3 | 87.8 |
| 5 | 2_9_130_168_246 | 95.9 | 84.0 | 87.5 |
| 5 | 13_18_121_130_165 | 98.4 | 82.5 | 87.2 |
| 5 | 13_18_130_165_268 | 98.6 | 82.3 | 87.2 |
| 5 | 4_17_115_130_168 | 92.7 | 84.3 | 86.8 |
| 5 | 2_4_130_168_173 | 93.7 | 83.9 | 86.8 |
| 5 | 2_9_130_168_173 | 93.7 | 83.2 | 86.3 |
| 5 | 2_111_130_168_173 | 93.3 | 83.0 | 86.1 |
| 5 | 2_83_130_168_173 | 94.5 | 82.2 | 85.9 |
| 5 | 2_6_130_168_173 | 94.5 | 82.0 | 85.7 |
| 5 | 2_6_130_173_184 | 95.9 | 81.3 | 85.6 |
| 5 | 2_130_168_173_213 | 94.9 | 81.4 | 85.4 |
| 5 | 2_5_130_168_173 | 92.9 | 82.1 | 85.3 |
| 5 | 2_130_168_173_249 | 93.9 | 81.5 | 85.2 |

TABLE 7-4

| Combined | | Validation cohort | | |
|---|---|---|---|---|
| gene number | SEQ ID NO: | Sensitivity | Specificity | Accuracy |
| 4 | 2_121_165_168 | 95.3 | 82.5 | 86.3 |
| 4 | 2_165_168_268 | 94.9 | 82.0 | 85.9 |
| 4 | 2_4_168_246 | 93.3 | 82.8 | 85.9 |
| 4 | 2_18_165_268 | 95.5 | 81.7 | 85.8 |
| 4 | 2_4_173_183 | 93.3 | 82.2 | 85.5 |
| 4 | 2_4_115_168 | 90.8 | 83.3 | 85.5 |
| 4 | 2_9_168_246 | 93.3 | 82.1 | 85.5 |
| 4 | 2_111_168_246 | 92.7 | 82.3 | 85.4 |
| 4 | 2_111_168_173 | 92.9 | 82.1 | 85.3 |
| 4 | 2_102_168_246 | 93.1 | 81.7 | 85.1 |
| 5 | 2_4_130_168_246 | 95.9 | 84.3 | 87.8 |
| 5 | 2_4_168_201_246 | 94.5 | 84.7 | 87.6 |
| 5 | 2_9_130_168_246 | 95.9 | 84.0 | 87.5 |
| 5 | 2_4_111_168_173 | 92.7 | 84.5 | 86.9 |
| 5 | 2_4_130_168_173 | 93.7 | 83.9 | 86.8 |
| 5 | 2_4_168_173_201 | 93.5 | 83.7 | 86.6 |
| 5 | 2_4_115_168_173 | 94.3 | 83.3 | 86.6 |
| 5 | 2_9_130_168_173 | 93.7 | 83.2 | 86.3 |
| 5 | 2_4_17_115_168 | 92.5 | 83.6 | 86.3 |
| 5 | 2_111_168_173_268 | 93.7 | 83.1 | 86.3 |
| 5 | 2_18_165_268_301 | 95.3 | 82.2 | 86.1 |
| 5 | 2_18_165_268_315 | 96.3 | 81.8 | 86.1 |
| 5 | 2_111_130_168_173 | 93.3 | 83.0 | 86.1 |
| 5 | 2_83_130_168_173 | 94.5 | 82.2 | 85.9 |
| 5 | 2_6_130_168_173 | 94.5 | 82.0 | 85.7 |
| 5 | 2_111_168_173_223 | 92.7 | 82.7 | 85.7 |
| 5 | 2_5_111_168_173 | 92.1 | 83.0 | 85.7 |
| 5 | 2_6_130_173_184 | 95.9 | 81.3 | 85.6 |
| 5 | 2_39_111_168_173 | 93.3 | 82.3 | 85.6 |
| 5 | 2_111_168_173_222 | 93.9 | 82.0 | 85.5 |
| 5 | 2_111_152_168_173 | 93.5 | 82.0 | 85.5 |
| 5 | 2_111_168_173_241 | 93.1 | 82.2 | 85.5 |
| 5 | 2_130_168_173_213 | 94.9 | 81.4 | 85.4 |
| 5 | 2_111_168_173_184 | 94.1 | 81.7 | 85.4 |
| 5 | 2_102_111_168_173 | 92.3 | 82.5 | 85.4 |
| 5 | 2_5_130_168_173 | 92.9 | 82.1 | 85.3 |
| 5 | 2_111_168_173_234 | 92.3 | 82.4 | 85.3 |
| 5 | 2_111_168_173_230 | 93.1 | 82.0 | 85.3 |
| 5 | 2_111_168_173_307 | 93.3 | 81.9 | 85.3 |
| 5 | 2_130_168_173_249 | 93.9 | 81.5 | 85.2 |
| 5 | 2_111_158_168_173 | 92.9 | 81.8 | 85.1 |
| 5 | 2_39_168_169_173 | 93.3 | 81.5 | 85.1 |

TABLE 7-5

| Combined | | Validation cohort | | |
|---|---|---|---|---|
| gene number | SEQ ID NO: | Sensitivity | Specificity | Accuracy |
| 3 | 9_165_168 | 95.9 | 83.0 | 86.9 |
| 4 | 9_165_168_173 | 96.5 | 83.2 | 87.2 |
| 4 | 9_128_165_168 | 95.3 | 83.6 | 87.1 |
| 4 | 9_17_165_168 | 94.7 | 83.5 | 86.9 |
| 4 | 9_80_165_168 | 97.4 | 82.2 | 86.8 |
| 4 | 2_9_168_246 | 93.3 | 82.1 | 85.5 |
| 5 | 5_9_165_168_173 | 96.5 | 83.9 | 87.6 |
| 5 | 9_128_129_165_168 | 94.5 | 84.6 | 87.5 |
| 5 | 2_9_130_168_246 | 95.9 | 84.0 | 87.5 |
| 5 | 9_17_159_165_168 | 94.7 | 83.4 | 86.8 |
| 5 | 9_17_165_168_173 | 95.1 | 83.1 | 86.7 |
| 5 | 2_9_130_168_173 | 93.7 | 83.2 | 86.3 |

TABLE 7-6

| Combined | | Validation cohort | | |
|---|---|---|---|---|
| gene number | SEQ ID NO: | Sensitivity | Specificity | Accuracy |
| 3 | 17_164_168 | 97.8 | 80.4 | 85.6 |
| 4 | 4_17_165_168 | 95.1 | 84.2 | 87.5 |
| 4 | 9_17_165_168 | 94.7 | 83.5 | 86.9 |
| 4 | 4_17_115_168 | 92.1 | 83.3 | 85.9 |
| 4 | 4_17_115_302 | 89.8 | 84.1 | 85.8 |
| 4 | 4_17_115_184 | 91.5 | 82.7 | 85.3 |
| 5 | 4_17_165_168_173 | 96.5 | 84.1 | 87.8 |
| 5 | 4_17_165_168_223 | 95.7 | 84.5 | 87.8 |
| 5 | 4_17_128_165_168 | 95.5 | 84.4 | 87.7 |
| 5 | 4_17_165_168_169 | 95.3 | 84.5 | 87.7 |
| 5 | 4_17_117_165_168 | 95.3 | 84.4 | 87.6 |
| 5 | 4_17_165_168_323 | 95.3 | 84.3 | 87.6 |
| 5 | 4_17_81_165_168 | 94.7 | 84.6 | 87.6 |
| 5 | 4_17_165_168_253 | 95.1 | 84.4 | 87.6 |
| 5 | 4_17_162_165_168 | 95.5 | 84.2 | 87.6 |
| 5 | 4_17_141_165_168 | 94.7 | 84.5 | 87.5 |
| 5 | 4_17_129_165_168 | 94.3 | 84.6 | 87.5 |
| 5 | 4_17_165_168_258 | 94.7 | 84.4 | 87.5 |
| 5 | 4_17_165_168_190 | 95.7 | 84.0 | 87.5 |
| 5 | 4_17_115_168_177 | 92.7 | 85.3 | 87.5 |
| 5 | 4_17_165_168_191 | 94.9 | 84.2 | 87.4 |
| 5 | 4_17_158_165_168 | 95.3 | 84.1 | 87.4 |
| 5 | 4_17_165_168_184 | 95.7 | 83.9 | 87.4 |
| 5 | 4_17_94_165_168 | 95.3 | 84.1 | 87.4 |
| 5 | 4_17_165_168_296 | 95.5 | 83.9 | 87.4 |
| 5 | 4_17_165_168_307 | 95.1 | 84.1 | 87.4 |
| 5 | 4_17_123_165_168 | 95.5 | 83.9 | 87.4 |
| 5 | 4_17_39_165_168 | 94.9 | 84.1 | 87.4 |
| 5 | 4_17_145_165_168 | 95.3 | 83.9 | 87.3 |
| 5 | 4_17_165_168_286 | 94.9 | 84.1 | 87.3 |
| 5 | 4_17_73_165_168 | 95.3 | 83.8 | 87.2 |
| 5 | 4_17_115_165_168 | 94.9 | 84.0 | 87.2 |
| 5 | 4_17_108_165_168 | 94.7 | 84.1 | 87.2 |
| 5 | 4_17_156_165_168 | 94.9 | 84.0 | 87.2 |
| 5 | 4_17_165_168_249 | 95.5 | 83.6 | 87.2 |
| 5 | 4_17_131_165_168 | 95.3 | 83.7 | 87.2 |
| 5 | 4_17_165_168_304 | 94.9 | 83.9 | 87.2 |
| 5 | 4_17_157_165_168 | 95.1 | 83.8 | 87.2 |
| 5 | 4_17_165_168_318 | 94.9 | 83.8 | 87.1 |
| 5 | 4_17_74_165_168 | 94.3 | 84.1 | 87.1 |
| 5 | 4_17_165_168_216 | 94.9 | 83.6 | 87.0 |
| 5 | 4_17_165_168_309 | 94.5 | 83.8 | 87.0 |
| 5 | 4_17_165_168_236 | 94.7 | 83.7 | 87.0 |
| 5 | 4_17_165_168_324 | 95.3 | 83.5 | 87.0 |
| 5 | 9_17_159_165_168 | 94.7 | 83.4 | 86.8 |
| 5 | 4_17_115_130_168 | 92.7 | 84.3 | 86.8 |
| 5 | 4_17_111_115_168 | 91.9 | 84.6 | 86.8 |
| 5 | 9_17_165_168_173 | 95.1 | 83.1 | 86.7 |
| 5 | 4_17_115_160_168 | 92.1 | 84.3 | 86.6 |
| 5 | 4_17_115_168_246 | 94.1 | 83.5 | 86.6 |
| 5 | 4_17_115_168_201 | 92.3 | 84.1 | 86.5 |
| 5 | 4_17_115_168_217 | 92.9 | 83.8 | 86.5 |
| 5 | 2_4_17_115_168 | 92.5 | 83.6 | 86.3 |
| 5 | 4_17_115_140_168 | 90.9 | 84.1 | 86.1 |
| 5 | 4_17_102_115_168 | 92.1 | 83.3 | 85.9 |

TABLE 7-7

| Combined | | Validation cohort | | |
|---|---|---|---|---|
| gene number | SEQ ID NO: | Sensitivity | Specificity | Accuracy |
| 3 | 121_130_164 | 97.6 | 82.3 | 86.9 |
| 3 | 18_121_164 | 98.6 | 81.6 | 86.7 |
| 3 | 121_164_168 | 97.8 | 80.9 | 85.9 |
| 3 | 121_164_328 | 96.3 | 81.1 | 85.6 |
| 3 | 121_164_211 | 97.6 | 80.2 | 85.4 |
| 3 | 95_121_164 | 98.4 | 79.7 | 85.3 |
| 3 | 6_121_165 | 96.3 | 80.3 | 85.1 |
| 4 | 18_121_130_164 | 98.6 | 84.4 | 88.6 |
| 4 | 18_121_164_255 | 98.0 | 84.2 | 88.4 |
| 4 | 18_121_164_201 | 98.2 | 83.0 | 87.6 |
| 4 | 18_121_151_164 | 98.2 | 83.1 | 87.6 |
| 4 | 18_95_121_164 | 99.0 | 81.5 | 86.8 |
| 4 | 18_107_121_164 | 98.6 | 81.6 | 86.7 |
| 4 | 2_121_165_168 | 95.3 | 82.5 | 86.3 |
| 4 | 18_121_149_165 | 97.1 | 81.7 | 86.3 |
| 4 | 13_18_121_165 | 99.2 | 80.4 | 86.1 |
| 5 | 18_121_130_136_164 | 98.6 | 84.6 | 88.8 |
| 5 | 18_121_130_164_314 | 98.4 | 84.6 | 88.8 |
| 5 | 18_114_121_130_164 | 99.0 | 84.3 | 88.7 |
| 5 | 18_121_130_164_214 | 98.6 | 84.5 | 88.7 |
| 5 | 18_121_130_164_193 | 98.8 | 84.3 | 88.6 |
| 5 | 18_121_130_164_320 | 98.6 | 84.4 | 88.6 |
| 5 | 18_121_130_164_301 | 98.6 | 84.3 | 88.6 |
| 5 | 18_121_130_144_164 | 98.6 | 84.4 | 88.6 |
| 5 | 18_121_130_164_168 | 98.4 | 84.5 | 88.6 |
| 5 | 18_121_130_164_205 | 98.4 | 84.4 | 88.6 |
| 5 | 18_121_130_158_164 | 98.8 | 84.1 | 88.5 |
| 5 | 18_121_130_164_260 | 98.8 | 84.1 | 88.5 |
| 5 | 18_106_121_130_164 | 98.8 | 84.1 | 88.5 |
| 5 | 18_121_130_164_318 | 98.6 | 84.2 | 88.5 |
| 5 | 18_121_130_164_286 | 98.6 | 84.1 | 88.5 |
| 5 | 18_121_130_164_315 | 98.6 | 84.2 | 88.5 |
| 5 | 18_121_130_164_237 | 98.6 | 84.2 | 88.5 |
| 5 | 18_121_130_164_184 | 98.6 | 84.2 | 88.5 |
| 5 | 18_121_130_164_270 | 98.4 | 84.2 | 88.5 |
| 5 | 18_121_130_164_309 | 98.4 | 84.2 | 88.5 |
| 5 | 18_121_130_164_278 | 98.4 | 84.3 | 88.5 |
| 5 | 18_82_121_130_164 | 98.4 | 84.2 | 88.5 |
| 5 | 18_23_121_130_164 | 98.4 | 84.2 | 88.5 |
| 5 | 18_121_130_164_189 | 98.4 | 84.3 | 88.5 |
| 5 | 18_121_130_152_164 | 98.2 | 84.3 | 88.5 |
| 5 | 18_121_130_164_213 | 98.2 | 84.3 | 88.5 |
| 5 | 18_121_130_164_229 | 98.2 | 84.3 | 88.5 |
| 5 | 18_57_121_130_164 | 98.2 | 84.4 | 88.5 |
| 5 | 18_121_130_142_164 | 98.8 | 83.9 | 88.4 |
| 5 | 18_121_130_155_164 | 98.6 | 84.0 | 88.4 |
| 5 | 18_39_121_130_164 | 98.4 | 84.1 | 88.4 |
| 5 | 18_33_121_130_164 | 98.4 | 84.1 | 88.4 |
| 5 | 18_121_126_130_164 | 98.4 | 84.1 | 88.4 |
| 5 | 18_121_130_164_319 | 98.4 | 84.1 | 88.4 |
| 5 | 18_22_121_130_164 | 98.2 | 84.1 | 88.4 |
| 5 | 18_59_121_130_164 | 98.2 | 84.1 | 88.4 |
| 5 | 18_27_121_130_164 | 97.8 | 84.4 | 88.4 |
| 5 | 18_121_130_164_201 | 98.2 | 84.1 | 88.3 |
| 5 | 18_19_121_130_164 | 98.4 | 83.9 | 88.2 |
| 5 | 18_95_121_164_188 | 99.0 | 83.0 | 87.8 |
| 5 | 13_18_121_130_165 | 98.4 | 82.5 | 87.2 |

### Example 3

<Comparison of miRNA Expression Levels in Serum Between Lung Cancer Patient and Healthy Subject>

In this Example, miRNA expression levels in sera were compared between lung cancer patients and healthy subjects in order to verify the reliability of the gene markers obtained in Examples 1 and 2. In this experiment, because higher statistical reliability regarding gene expression levels could be obtained when a larger number of samples is used, all the samples in which the gene expression levels were measured in the preceding Reference Examples were used (Table 11a). To be more specific, firstly, the miRNA expression levels of 1,694 lung cancer patients and 4,660 healthy subjects obtained in the preceding Reference Examples were combined and normalized by global normalization. Secondly, in order to evaluate diagnostic markers with higher reliability, only genes having the expression level of 2⁶ or higher in 50% or more of the samples in either of the lung cancer patient group or the healthy subject group, were selected. Thirdly, in order to evaluate a gene whose expression level significantly differs in statistics between a lung cancer patient group and the healthy subject group, a two-sided t-test assuming equal variance was carried out, and then, a P value after the Bonferroni correction was calculated. Forthly, in order to evaluate whether to be easily affected by noise at the time of measurement, an absolute value of the difference (fold change) in gene expression level, which is obtained by logarithmic conversion between the lung cancer patient group and the healthy subject group, was calculated. Genes having a P value after the correction which was 0.05 or less and having an absolute value of fold change which was 0.5 or more, were extracted as genes varying in expression. The results are shown in Table 8.

TABLE 8

| | P value after | Fold change of |
|---|---|---|
| | Bonferroni | lung cancer patient to |
| SEQ ID NO: | correction | healthy subject |
| 1 | <1E−308 | 2.02 |
| 2 | <1E−308 | 2.95 |
| 3 | <1E−308 | 1.63 |
| 7 | <1E−308 | 0.81 |
| 10 | 5.30E−284 | 0.67 |
| 12 | <1E−308 | 2.04 |
| 13 | <1E−308 | 7.81 |
| 14 | 4.44E−148 | −0.52 |
| 15 | <1E−308 | 1.00 |
| 16 | <1E−308 | 1.41 |
| 17 | <1E−308 | 0.86 |
| 18 | <1E−308 | 0.85 |
| 19 | 4.47E−85 | 0.65 |
| 20 | <1E−308 | 1.29 |
| 21 | <1E−308 | 1.85 |
| 22 | <1E−308 | 3.64 |
| 23 | <1E−308 | 1.60 |
| 25 | <1E−308 | 3.01 |
| 26 | <1E−308 | 1.87 |
| 27 | <1E−308 | 3.01 |
| 29 | <1E−308 | 1.91 |
| 30 | <1E−308 | 4.83 |
| 31 | <1E−308 | 3.23 |
| 33 | <1E−308 | 1.53 |
| 34 | 3.29E−303 | 0.82 |
| 35 | <1E−308 | −0.54 |
| 36 | 2.02E−291 | 0.99 |
| 37 | <1E−308 | 1.21 |
| 38 | <1E−308 | 1.53 |
| 40 | <1E−308 | 3.15 |
| 42 | <1E−308 | −0.94 |
| 43 | <1E−308 | 2.13 |
| 44 | <1E−308 | 1.95 |
| 45 | <1E−308 | 2.38 |
| 46 | <1E−308 | 0.58 |
| 47 | <1E−308 | 1.30 |
| 50 | <1E−308 | 4.63 |
| 51 | <1E−308 | 1.09 |
| 55 | <1E−308 | 0.68 |
| 56 | <1E−308 | 1.19 |
| 57 | <1E−308 | 3.71 |
| 58 | <1E−308 | −0.92 |
| 59 | <1E−308 | 1.27 |
| 60 | <1E−308 | 3.87 |
| 64 | <1E−308 | 4.03 |
| 65 | <1E−308 | 0.58 |
| 66 | <1E−308 | 1.62 |
| 67 | <1E−308 | 0.60 |
| 68 | <1E−308 | 3.95 |
| 69 | <1E−308 | 3.20 |
| 70 | <1E−308 | 4.36 |
| 71 | <1E−308 | 0.81 |
| 72 | <1E−308 | 3.83 |
| 73 | <1E−308 | −2.42 |
| 74 | <1E−308 | 0.97 |
| 75 | <1E−308 | 3.76 |
| 76 | <1E−308 | 1.16 |
| 78 | <1E−308 | 4.29 |
| 79 | 7.77E−294 | 0.77 |
| 80 | <1E−308 | 2.72 |
| 81 | <1E−308 | 1.08 |
| 84 | <1E−308 | −0.80 |
| 85 | <1E−308 | 3.89 |
| 86 | 4.63E−209 | 0.57 |
| 87 | <1E−308 | 3.01 |
| 90 | <1E−308 | 0.67 |
| 92 | <1E−308 | 0.86 |
| 94 | <1E−308 | 2.29 |
| 95 | <1E−308 | 0.75 |
| 96 | <1E−308 | 3.61 |
| 97 | <1E−308 | 1.15 |
| 98 | <1E−308 | 3.35 |
| 99 | <1E−308 | 1.22 |
| 101 | <1E−308 | 0.87 |
| 102 | <1E−308 | 0.58 |
| 103 | <1E−308 | 0.85 |
| 104 | <1E−308 | 0.78 |
| 105 | <1E−308 | 0.75 |
| 106 | <1E−308 | 1.77 |
| 107 | <1E−308 | 1.61 |
| 109 | <1E−308 | −0.58 |
| 110 | <1E−308 | 0.89 |
| 111 | <1E−308 | 0.72 |
| 113 | 8.52E−107 | 0.52 |
| 114 | 3.97E−267 | 0.63 |
| 115 | <1E−308 | 1.51 |
| 118 | <1E−308 | −0.80 |
| 120 | <1E−308 | 1.56 |
| 122 | <1E−308 | 2.26 |
| 124 | <1E−308 | 1.24 |
| 127 | <1E−308 | 1.40 |
| 128 | <1E−308 | 1.05 |
| 130 | <1E−308 | 0.55 |
| 131 | <1E−308 | −1.09 |
| 134 | <1E−308 | 1.38 |
| 135 | <1E−308 | 3.67 |
| 136 | <1E−308 | −1.41 |
| 137 | <1E−308 | 2.98 |
| 138 | 9.35E−164 | 0.54 |
| 140 | 8.27E−232 | 0.63 |
| 142 | 7.60E−293 | 0.73 |
| 143 | <1E−308 | 2.39 |
| 144 | <1E−308 | 1.12 |
| 145 | <1E−308 | −1.21 |
| 148 | <1E−308 | 1.55 |
| 149 | <1E−308 | 3.33 |
| 150 | <1E−308 | 4.64 |
| 153 | <1E−308 | 2.85 |
| 159 | <1E−308 | 1.19 |
| 162 | <1E−308 | 2.23 |
| 163 | <1E−308 | 3.49 |
| 164 | <1E−308 | 3.66 |
| 165 | <1E−308 | 1.98 |
| 166 | 3.76E−62 | −0.65 |
| 167 | 2.34E−248 | 0.61 |
| 170 | <1E−308 | 4.76 |
| 172 | <1E−308 | 1.30 |
| 173 | <1E−308 | 1.13 |
| 175 | <1E−308 | −1.26 |
| 177 | <1E−308 | 1.68 |
| 179 | <1E−308 | 1.49 |
| 180 | <1E−308 | 0.90 |
| 181 | <1E−308 | 1.16 |
| 182 | <1E−308 | 2.32 |
| 183 | <1E−308 | −0.85 |
| 184 | <1E−308 | −0.69 |
| 185 | <1E−308 | 1.96 |
| 186 | <1E−308 | −0.95 |
| 187 | <1E−308 | 1.14 |
| 188 | <1E−308 | 4.99 |
| 190 | <1E−308 | 4.22 |
| 193 | <1E−308 | 0.65 |
| 195 | <1E−308 | 5.97 |
| 196 | <1E−308 | 4.90 |
| 197 | <1E−308 | 5.16 |
| 198 | <1E−308 | 3.04 |
| 199 | <1E−308 | 2.28 |
| 200 | <1E−308 | 0.74 |
| 201 | <1E−308 | 1.42 |
| 202 | <1E−308 | 0.72 |
| 206 | <1E−308 | 2.54 |
| 207 | <1E−308 | 2.68 |
| 209 | <1E−308 | 1.03 |
| 211 | <1E−308 | 1.06 |
| 214 | <1E−308 | 1.09 |
| 215 | <1E−308 | −1.12 |
| 217 | <1E−308 | 1.32 |
| 220 | <1E−308 | 2.71 |
| 221 | <1E−308 | 0.79 |
| 222 | <1E−308 | 0.64 |
| 225 | <1E−308 | 3.19 |
| 226 | <1E−308 | 3.05 |
| 229 | 4.55E−203 | −0.63 |
| 231 | <1E−308 | 5.80 |
| 232 | <1E−308 | 1.10 |
| 235 | 7.77E−196 | −0.55 |
| 236 | <1E−308 | 1.52 |
| 239 | <1E−308 | 0.95 |
| 246 | <1E−308 | 1.36 |
| 247 | <1E−308 | 1.17 |
| 249 | 3.06E−90 | −0.65 |
| 250 | <1E−308 | 0.81 |
| 251 | <1E−308 | 1.01 |
| 255 | <1E−308 | 3.10 |
| 256 | 3.79E−269 | 0.56 |
| 257 | <1E−308 | 1.99 |
| 259 | <1E−308 | 1.72 |
| 260 | <1E−308 | 3.26 |
| 261 | <1E−308 | 1.88 |
| 262 | <1E−308 | 1.54 |
| 263 | <1E−308 | 3.49 |
| 265 | 2.97E−266 | 0.55 |
| 267 | <1E−308 | 0.91 |
| 268 | 1.19E−234 | 0.55 |
| 269 | <1E−308 | 0.77 |
| 272 | <1E−308 | 1.43 |
| 273 | 1.04E−304 | 0.57 |
| 274 | <1E−308 | −0.92 |
| 276 | <1E−308 | 0.73 |
| 277 | 6.62E−218 | 0.64 |
| 278 | <1E−308 | −1.41 |
| 279 | <1E−308 | −0.83 |
| 280 | 1.54E−225 | 0.57 |
| 281 | <1E−308 | 1.05 |
| 282 | <1E−308 | −0.50 |
| 284 | 2.35E−278 | −0.60 |
| 285 | <1E−308 | 1.67 |
| 286 | <1E−308 | −1.27 |
| 287 | <1E−308 | 1.31 |
| 290 | <1E−308 | 1.08 |
| 291 | <1E−308 | 1.57 |
| 293 | <1E−308 | −0.51 |
| 294 | 2.90E−300 | 0.64 |
| 295 | <1E−308 | 1.12 |
| 296 | 5.92E−276 | −0.64 |
| 297 | 6.60E−269 | −0.60 |
| 298 | <1E−308 | 0.87 |
| 299 | <1E−308 | 2.46 |
| 300 | <1E−308 | 2.52 |
| 301 | <1E−308 | 0.87 |
| 303 | <1E−308 | 0.88 |
| 304 | <1E−308 | −1.68 |
| 305 | <1E−308 | 0.87 |
| 306 | <1E−308 | 1.06 |
| 307 | <1E−308 | 0.79 |
| 309 | <1E−308 | 3.01 |
| 311 | <1E−308 | 5.07 |
| 312 | <1E−308 | 4.83 |
| 313 | <1E−308 | 1.14 |
| 314 | <1E−308 | 0.75 |
| 315 | <1E−308 | 2.69 |
| 316 | <1E−308 | 1.63 |
| 317 | <1E−308 | 0.97 |
| 318 | <1E−308 | 3.19 |
| 319 | <1E−308 | 5.62 |
| 320 | <1E−308 | 1.02 |
| 321 | <1E−308 | 1.24 |
| 322 | <1E−308 | 2.07 |
| 324 | <1E−308 | −1.84 |
| 327 | 5.87E−261 | 1.52 |
| 328 | <1E−308 | 2.66 |
| 329 | <1E−308 | 3.30 |

### Example 4

<Comparison of miRNA Expression Levels in Serum Between Lung Cancer Patient and Benign Bone and Soft Tissue Tumor Patients and Benign Breast Disease Patients>

In this Example, miRNA expression levels in sera were compared between lung cancer patients and benign bone and soft tissue tumor patients and benign breast disease patients in order to verify the reliability of the gene markers obtained in Examples 1 and 2. In this experiment, because higher statistical reliability regarding gene expression levels could be obtained when a larger number of samples is used, all the samples in which the gene expression levels were measured in the preceding Reference Examples were used (Table 11a). To be more specific, firstly, the miRNA expression levels of 1,694 lung cancer patients and 368 benign bone and soft tissue tumor patients and benign breast disease patients obtained in the preceding Reference Examples were combined and normalized by global normalization.

Secondly, in order to evaluate diagnostic markers with higher reliability, only genes having the expression level of 2⁶ or higher in 50% or more of the samples in either of the group of lung cancer patients or the group of benign bone and soft tissue tumor patients and benign breast disease patients, were selected. Thirdly, in order to evaluate a gene whose expression level significantly differs in statistics between the group of lung cancer patients and the group of benign bone and soft tissue tumor patients and benign breast disease patients, a two-sided t-test assuming equal variance was carried out, and then, a P value after the Bonferroni correction was calculated. Forthly, in order to evaluate whether to be easily affected by noise at the time of measurement, an absolute value of the difference (fold change) in gene expression level, which is obtained by logarithmic conversion between the group of lung cancer patients and the group of benign bone and soft tissue tumor patients and benign breast disease patients, was calculated. A gene having a P value after the correction which was 0.05 or less and having an absolute value of fold change which was 0.5 or more, was extracted as a gene varying in expression. The results are shown in Table 9.

TABLE 9

| | | Fold change of |
|---|---|---|
| | | lung cancer patient to |
| | | benign bone and soft |
| | P value after | tissue tumor patients |
| | Bonferroni | and benign |
| SEQ ID NO: | correction | breast disease patients |
| 1 | 2.39E−129 | 0.94 |
| 2 | 1.31E−199 | 2.34 |
| 3 | 7.47E−167 | 1.49 |
| 4 | 3.64E−68 | −1.48 |
| 8 | 2.49E−75 | 1.03 |
| 9 | 9.04E−40 | −1.17 |
| 11 | 8.51E−43 | −0.55 |
| 12 | 7.12E−163 | 1.24 |
| 13 | 1.57E−149 | 2.56 |
| 15 | 1.26E−108 | 1.26 |
| 20 | 5.02E−73 | 1.03 |
| 22 | 2.96E−67 | 1.33 |
| 23 | 2.55E−128 | 1.57 |
| 24 | 7.59E−83 | −0.55 |
| 25 | 1.04E−58 | 1.12 |
| 27 | 1.98E−75 | 1.76 |
| 29 | 2.82E−175 | 1.91 |
| 30 | 2.35E−57 | 1.78 |
| 31 | 9.31E−74 | 1.14 |
| 32 | 3.51E−288 | −0.51 |
| 33 | 2.39E−102 | 1.50 |
| 34 | 1.65E−117 | 0.87 |
| 36 | 2.38E−48 | 0.85 |
| 40 | 7.11E−197 | 2.24 |
| 43 | 4.45E−73 | 0.88 |
| 44 | 6.93E−34 | 0.88 |
| 45 | 1.20E−84 | 0.83 |
| 47 | 2.20E−55 | 0.84 |
| 48 | 7.93E−64 | 0.79 |
| 50 | 4.47E−86 | 1.69 |
| 56 | 6.73E−93 | 0.90 |
| 57 | 6.51E−65 | 1.24 |
| 59 | 1.22E−39 | 0.85 |
| 60 | 6.33E−158 | 2.26 |
| 64 | 8.48E−18 | 1.00 |
| 66 | 1.35E−144 | 0.77 |
| 68 | 4.04E−101 | 1.61 |
| 69 | 1.85E−27 | 1.00 |
| 70 | 5.91E−114 | 1.85 |
| 72 | 1.62E−91 | 1.67 |
| 73 | 2.55E−32 | 0.62 |
| 75 | 2.81E−85 | 1.55 |
| 76 | 4.11E−23 | 0.59 |
| 78 | 2.75E−73 | 1.79 |
| 79 | 1.95E−45 | 0.85 |
| 80 | 1.21E−150 | 1.33 |
| 85 | <1E−308 | 4.19 |
| 87 | 2.13E−115 | 1.66 |
| 94 | 7.34E−131 | 1.46 |
| 96 | 8.87E−95 | 1.42 |
| 98 | 4.83E−171 | 2.02 |
| 99 | 1.02E−39 | 0.84 |
| 102 | 6.67E−165 | 1.11 |
| 106 | 5.24E−29 | 0.50 |
| 107 | 7.47E−61 | 0.67 |
| 110 | 5.98E−82 | 0.53 |
| 114 | 2.73E−20 | 0.52 |
| 115 | 8.65E−115 | 0.54 |
| 120 | 3.91E−46 | 0.81 |
| 122 | 4.73E−52 | 1.17 |
| 126 | 1.19E−154 | −0.53 |
| 128 | 9.40E−33 | 0.64 |
| 134 | 1.36E−41 | 0.72 |
| 135 | 5.46E−172 | 2.32 |
| 136 | 4.38E−35 | −0.70 |
| 137 | <1E−308 | 1.71 |
| 138 | 9.15E−27 | 0.64 |
| 139 | 9.22E−34 | 0.62 |
| 140 | 1.08E−44 | 0.74 |
| 143 | 2.03E−70 | 1.52 |
| 144 | 3.76E−22 | 0.62 |
| 148 | 1.34E−47 | 0.66 |
| 149 | 8.40E−110 | 1.76 |
| 150 | 1.98E−89 | 1.68 |
| 153 | 1.39E−161 | 2.04 |
| 159 | 1.11E−50 | 0.74 |
| 163 | 1.05E−120 | 1.98 |
| 164 | <1E−308 | 2.51 |
| 165 | 9.44E−220 | 1.28 |
| 166 | 5.85E−66 | −1.73 |
| 167 | 6.22E−235 | 1.31 |
| 170 | 7.90E−67 | 1.84 |
| 172 | 2.04E−94 | 1.31 |
| 173 | 9.00E−61 | 0.60 |
| 179 | 5.55E−45 | 0.55 |
| 181 | 7.23E−54 | 0.81 |
| 182 | 1.03E−104 | 0.81 |
| 185 | 1.24E−82 | 1.06 |
| 186 | 8.97E−59 | 0.86 |
| 188 | <1E−308 | 6.00 |
| 189 | 6.66E−102 | 1.18 |
| 190 | 2.48E−177 | 2.08 |
| 194 | 3.50E−122 | 0.67 |
| 195 | 6.34E−123 | 1.89 |
| 196 | 1.24E−104 | 2.05 |
| 197 | 5.84E−97 | 2.35 |
| 198 | 1.14E−114 | 1.54 |
| 199 | 8.95E−90 | 0.93 |
| 201 | 1.58E−195 | 1.24 |
| 202 | 6.22E−55 | 0.58 |
| 206 | 2.35E−30 | 0.56 |
| 209 | 2.52E−75 | 0.52 |
| 213 | 1.88E−68 | −0.66 |
| 217 | 3.25E−133 | 0.94 |
| 220 | 2.19E−173 | 1.30 |
| 225 | 4.62E−84 | 1.58 |
| 226 | 8.00E−184 | 1.32 |
| 228 | 1.25E−59 | 1.42 |
| 231 | <1E−308 | 4.03 |
| 235 | 2.68E−20 | 0.52 |
| 241 | 2.57E−182 | −0.57 |
| 244 | 1.26E−14 | 0.53 |
| 249 | 2.46E−25 | −0.78 |
| 250 | 2.66E−89 | 0.58 |
| 255 | 8.95E−34 | 0.58 |
| 257 | 5.57E−54 | 1.34 |
| 260 | 4.52E−157 | 1.97 |
| 262 | 3.71E−31 | 0.53 |
| 263 | 4.51E−89 | 1.03 |
| 285 | 6.31E−83 | 1.16 |
| 287 | 2.36E−295 | 1.36 |
| 289 | 5.59E−114 | 1.04 |
| 291 | 4.37E−44 | 0.55 |
| 294 | 2.77E−20 | 0.51 |
| 299 | 6.64E−135 | 1.05 |
| 303 | 7.57E−59 | 0.95 |
| 311 | 2.95E−83 | 2.16 |
| 312 | 3.35E−238 | 2.89 |
| 313 | 1.44E−53 | 0.76 |
| 315 | 1.73E−20 | 0.68 |
| 319 | 7.52E−85 | 1.75 |
| 320 | 3.60E−29 | 0.71 |
| 322 | 9.40E−116 | 1.07 |
| 325 | 3.44E−130 | −0.55 |
| 327 | 2.41E−29 | 0.90 |
| 328 | 2.19E−125 | 1.08 |
| 329 | 2.60E−73 | 1.31 |

### Example 5

<Comparison of miRNA Expression Levels in Serum Between Lung Cancer Patient and Patient Having a Cancer Other than Lung Cancer>

In this Example, miRNA expression levels in sera were compared between lung cancer patients and other cancer patients in order to verify the reliability of the gene markers obtained in Examples 1 and 2. In this experiment, because higher statistical reliability regarding gene expression levels could be obtained when a larger number of samples is used, all the samples in which the gene expression levels were measured in the preceding Reference Examples, were used (Table 11a). To be specific, firstly, the miRNA expression levels of 1,694 lung cancer patients and 4,147 other cancer patients obtained in the preceding Reference Examples were combined and normalized by global normalization. Secondly, in order to evaluate diagnostic markers with higher reliability, only genes having the expression level of 2⁶ or higher in 50% or more of the samples in either of the group of lung cancer patients or the group of other cancer patients, were selected. Thirdly, in order to evaluate a gene whose gene expression level significantly differs in statistics between the group of lung cancer patients and the group of other cancer patients, a two-sided t-test assuming equal variance was carried out, and then, a P value after the Bonferroni correction was calculated. Forthly, in order to evaluate whether to be easily affected by noise at the time of measurement, an absolute value of the difference (fold change) in gene expression level, which is obtained by logarithmic conversion between the group of lung cancer patients and the group of patients having a cancer other than lung cancer, was calculated. A gene having a P value after the correction which was 0.05 or less and having an absolute value of fold change which was 0.5 or more, was extracted as a gene varying in expression. The results are shown in Table 10.

TABLE 10

| | | Fold change of |
|---|---|---|
| | P value after | lung cancer patient to |
| | Bonferroni | patient having cancer |
| SEQ ID NO: | correction | other than lung cancer |
| 1 | 4.58E−123 | 0.55 |
| 2 | 1.88E−145 | 1.27 |
| 3 | 6.31E−96 | 0.66 |
| 4 | 1.76E−150 | −1.08 |
| 9 | 8.54E−95 | −0.93 |
| 13 | 1.21E−114 | 1.38 |
| 20 | 1.20E−69 | 0.57 |
| 23 | 2.52E−48 | 0.53 |
| 29 | 3.77E−99 | 0.77 |
| 30 | 3.12E−43 | 0.83 |
| 33 | 1.61E−53 | 0.57 |
| 40 | 8.43E−82 | 0.78 |
| 50 | 2.76E−40 | 0.67 |
| 60 | 7.34E−122 | 1.10 |
| 64 | 4.67E−30 | 0.64 |
| 68 | 4.99E−91 | 0.92 |
| 69 | 9.36E−39 | 0.58 |
| 70 | 8.65E−59 | 0.77 |
| 72 | 5.35E−106 | 0.91 |
| 75 | 2.31E−52 | 0.67 |
| 78 | 3.67E−39 | 0.73 |
| 87 | 6.09E−57 | 0.66 |
| 94 | 1.00E−113 | 0.90 |
| 96 | 3.83E−68 | 0.65 |
| 98 | 6.51E−115 | 0.93 |
| 102 | 4.09E−92 | 0.51 |
| 120 | 2.93E−63 | 0.61 |
| 122 | 5.74E−73 | 0.76 |
| 135 | 2.97E−118 | 1.08 |
| 140 | 1.69E−67 | 0.56 |
| 143 | 1.34E−60 | 0.74 |
| 149 | 8.83E−80 | 0.88 |
| 150 | 2.47E−76 | 0.89 |
| 153 | 3.64E−76 | 0.80 |
| 163 | 1.53E−95 | 0.91 |
| 164 | 5.78E−212 | 0.92 |
| 165 | 2.37E−104 | 0.56 |
| 166 | 2.06E−83 | −1.03 |
| 170 | 9.80E−60 | 0.85 |
| 188 | 1.53E−44 | 0.57 |
| 195 | 1.39E−75 | 0.83 |
| 196 | 5.59E−82 | 1.00 |
| 197 | 1.10E−77 | 1.09 |
| 198 | 1.49E−75 | 0.74 |
| 220 | 6.63E−89 | 0.56 |
| 225 | 3.64E−51 | 0.67 |
| 228 | 1.25E−100 | 0.86 |
| 231 | 2.36E−114 | 1.27 |
| 260 | 8.14E−106 | 0.92 |
| 263 | 6.10E−65 | 0.66 |
| 311 | 6.14E−102 | 1.34 |
| 312 | 9.19E−63 | 0.80 |
| 319 | 4.08E−58 | 0.97 |
| 327 | 4.54E−51 | 0.69 |
| 329 | 4.55E−101 | 0.92 |

As shown in the above Examples, the kit, device and method of the present invention can detect lung adenocarcinoma, lung squamous cell carcinoma, large cell lung carcinoma, small cell lung carcinoma and other lung cancers with higher sensitivity than the existing tumor markers and therefore permit early detection of lung cancer. As a result, a treatment such as a chemotherapy, a radiotherapy, an immunotherapy, a molecular targeted therapy, or surgery with a high degree of probability for complete therapy can be applied early, thereby significantly improving a survival rate.

### INDUSTRIAL APPLICABILITY

According to the present invention, various histological types or stages of progression of lung cancer can be effectively detected by a simple and inexpensive method. This enables early detection, diagnosis and treatment of lung cancer. Also, the method of the present invention enables less-invasive detection of lung cancer using patient's blood and therefore lung cancer can be simply and quickly detected.

All publications, patents, and patent applications cited herein are incorporated herein by reference in their entirety.

1. A method for detecting and treating or performing a diagnostic procedure for lung cancer, comprising: determining an expression level of hsa-miR-3619-3p in a sample from a human subject using a nucleic acid(s) capable of specifically binding to hsa-miR-3619-3p, or using a kit or device comprising a nucleic acid(s) capable of specifically binding to
hsa-miR-3619-3p,
wherein the determining comprises the following steps of:
(a) isolating a sample from a human subject comprising hsa-miR-3619-3p;
(b) contacting hsa-miR-3619-3p in the sample or complementary polynucleotide(s) thereof prepared from hsa-miR-3619-3p with the nucleic acid(s);
(c) measuring an expression level of hsa-miR-3619-3p;
(d) comparing the expression level of hsa-miR-3619-3p measured in the step (c) to a control expression level of hsa-miR-3619-3p in a control sample from a healthy human subject measured in the same way as in the step (c) to allow for diagnosis of lung cancer;
(e) detecting a increased level of hsa-miR-3619-3p in the sample from the human subject as compared to the control expression level of hsa-miR-3619-3p from the sample from the human subject that does not have lung cancer, wherein the increased level of hsa-miR-3619-3p indicates that the human subject has lung cancer; and
(f) treating the human subject having lung cancer or performing a diagnostic procedure on the human subject having lung cancer;
wherein the sample is blood, serum, or plasma;
wherein the treatment comprises chemotherapy, radiotherapy, immunotherapy, molecular targeted therapy, surgery or a combination thereof; and
wherein the diagnostic procedure comprises an imaging test method selected from the group consisting of a chest X-ray examination, CT examination, MRI examination, and PET examination; a pathological examination method selected from the group consisting of sputum cytology, pleural fluid analysis, bronchoscopy, and percutaneous needle biopsy; or a combination thereof.

2. The method according to claim 1, wherein the nucleic acid(s) is a polynucleotide(s) selected from the group consisting of the following polynucleotides (a) to (e):
(a) a polynucleotide consisting of a nucleotide sequence of SEQ ID NO: 34, or a nucleotide sequence derived from the nucleotide sequence in which the nucleic acid u is replaced with t, a variant thereof, a derivative thereof, or a fragment thereof comprising 15 or more consecutive nucleotides;
(b) a polynucleotide comprising a nucleotide sequence of SEQ ID NO: 34;
(c) a polynucleotide consisting of a nucleotide sequence complementary to a nucleotide sequence of SEQ ID NO: 34, or a nucleotide sequence derived from the nucleotide sequence in which the nucleic acid u is replaced with t, a variant thereof, a derivative thereof, or a fragment thereof comprising 15 or more consecutive nucleotides;
(d) a polynucleotide comprising a nucleotide sequence complementary to a nucleotide sequence of SEQ ID NO: 34, or a nucleotide sequence derived from the nucleotide sequence in which the nucleic acid u is replaced with t; and
(e) a polynucleotide hybridizing under stringent conditions to any of the polynucleotides (a) to (d).

3. The method according to claim 1, wherein the method further comprises:
determining an expression level(s) of one or more other miRNA(s) selected from the following other lung cancer markers: miR-6787-5p, miR-920, miR-3622a-5p, miR-1185-1-3p, miR-4327, miR-5739, miR-937-5p, miR-1181, miR-1185-2-3p, miR-1193, miR-1207-5p, miR-1238-5p, miR-1246, miR-1249-5p, miR-1292-3p, miR-1469, miR-1470, miR-197-5p, miR-208a-5p, miR-2110, miR-211-3p, miR-3122, miR-3141, miR-3156-5p, miR-3158-5p, miR-3160-5p, miR-3180-3p, miR-3191-3p, miR-3194-3p, miR-320b, miR-328-5p, miR-3610, miR-3620-5p, miR-370-3p, miR-373-5p, miR-3917, miR-3937, miR-4259, miR-4281, miR-4294, miR-4419b, miR-4428, miR-4429, miR-4433a-3p, miR-4447, miR-4449, miR-4459, miR-4480, miR-4485-5p, miR-4486, miR-4488, miR-4489, miR-4505, miR-4513, miR-4515, miR-4530, miR-4535, miR-4635, miR-4640-5p, miR-4646-5p, miR-4656, miR-4663, miR-4665-5p, miR-4706, miR-4707-5p, miR-4708-3p, miR-4710, miR-4718, miR-4722-5p, miR-4727-3p, miR-4734, miR-4740-5p, miR-4747-3p, miR-4749-5p, miR-4755-3p, miR-4763-5p, miR-4787-3p, miR-5008-5p, miR-5010-5p, miR-504-3p, miR-5090, miR-5100, miR-5196-5p, miR-551b-5p, miR-557, miR-5787, miR-6090, miR-6124, miR-6132, miR-6510-5p, miR-6511b-5p, miR-6515-3p, miR-654-5p, miR-658, miR-668-5p, miR-6722-5p, miR-6724-5p, miR-6729-3p, miR-6737-5p, miR-6756-5p, miR-6762-5p, miR-6763-3p, miR-6766-5p, miR-6769a-5p, miR-6771-5p, miR-6786-5p, miR-6789-5p, miR-6794-5p, miR-6796-3p, miR-6797-5p, miR-6800-3p, miR-6802-5p, miR-6803-5p, miR-6805-3p, miR-6805-5p, miR-6807-5p, miR-6812-5p, miR-6819-5p, miR-6822-5p, miR-6824-5p, miR-6826-5p, miR-6850-5p, miR-6858-5p, miR-6861-5p, miR-6880-3p, miR-7107-5p, miR-7109-5p, miR-7114-5p, miR-7704, miR-7846-3p, miR-8052, miR-8060, miR-8071, miR-8073, miR-874-5p, miR-204-3p, miR-3154, miR-3960, miR-4433a-5p, miR-4455, miR-4462, miR-4476, miR-4508, miR-4687-3p, miR-4687-5p, miR-4732-5p, miR-4771, miR-642a-3p, miR-6732-5p, miR-6760-5p, miR-6799-5p, miR-6820-5p, miR-6821-5p, miR-6829-5p, miR-6893-5p, miR-7108-3p, miR-7111-5p, miR-8089, miR-885-3p, miR-92b-3p, miR-1343-3p, miR-6746-5p, miR-422a, miR-187-5p, miR-4632-5p, miR-6791-5p, miR-103a-3p, miR-107, miR-1199-5p, miR-1225-3p, miR-1225-5p, miR-1228-5p, miR-1229-5p, miR-1233-5p, miR-1237-5p, miR-1247-3p, miR-1249-3p, miR-1254, miR-1260b, miR-1268a, miR-1268b, miR-1273g-3p, miR-128-1-5p, miR-128-2-5p, miR-1290, miR-150-3p, miR-17-3p, miR-1908-5p, miR-1909-3p, miR-1914-3p, miR-1915-3p, miR-191-5p, miR-22-3p, miR-23b-3p, miR-24-3p, miR-296-3p, miR-296-5p, miR-3131, miR-3162-5p, miR-3188, miR-3196, miR-3197, miR-320a, miR-342-5p, miR-3621, miR-3648, miR-3656, miR-365a-5p, miR-3665, miR-3679-5p, miR-371a-5p, miR-3940-5p, miR-423-5p, miR-4257, miR-4270, miR-4271, miR-4286, miR-4298, miR-4417, miR-4442, miR-4446-3p, miR-4448, miR-4454, miR-4467, miR-4472, miR-4507, miR-4516, miR-451a, miR-4649-5p, miR-4651, miR-4665-3p, miR-4674, miR-4675, miR-4689, miR-4695-5p, miR-4697-5p, miR-4725-3p, miR-4739, miR-4745-5p, miR-4763-3p, miR-4792, miR-486-3p, miR-5001-5p, miR-5195-3p, miR-550a-5p, miR-5698, miR-6075, miR-6088, miR-6089, miR-6125, miR-6126, miR-614, miR-615-5p, miR-619-5p, miR-638, miR-642b-3p, miR-650, miR-663a, miR-663b, miR-6717-5p, miR-6721-5p, miR-6726-5p, miR-6727-5p, miR-6738-5p, miR-6741-5p, miR-6749-5p, miR-6752-5p, miR-675-5p, miR-6757-5p, miR-6763-5p, miR-6765-5p, miR-6775-5p, miR-6780b-5p, miR-6782-5p, miR-6784-5p, miR-6800-5p, miR-6806-5p, miR-6840-3p, miR-6848-5p, miR-6851-5p, miR-6870-5p, miR-6872-3p, miR-6875-5p, miR-6877-5p, miR-6879-5p, miR-6880-5p, miR-6885-5p, miR-6887-5p, miR-7108-5p, miR-711, miR-7113-3p, miR-744-5p, miR-760, miR-7845-5p, miR-7847-3p, miR-7977, miR-8059, miR-8063, miR-8072, miR-874-3p, miR-92a-2-5p, miR-92b-5p, miR-940, miR-1228-3p, miR-1275, miR-1307-3p, miR-1343-5p, miR-23a-3p, miR-29b-3p, miR-3135b, miR-3185, miR-4532, miR-4690-5p, miR-4758-5p, miR-4783-3p, miR-6131, miR-625-3p, miR-6511a-5p, miR-6765-3p, miR-6816-5p, miR-6825-5p, miR-6845-5p, miR-7150, miR-7641, miR-7975, and miR-92a-3p, or to a complementary strand of the polynucleotide, in the sample from the human subject by the same way as in the steps (b) and (c) using a nucleic acid(s) capable of specifically binding to the miRNA(s), or using a kit or device comprising a nucleic acid(s) capable of specifically binding to the miRNA(s).

4. The method according to claim 1, wherein the measuring in the step (c) is performed by quantitative RT-PCR using the nucleic acid(s) as primer(s).

5. The method according to claim 1, wherein the measuring in the step (c) is performed by hybridization using the nucleic acid(s) as probe(s).

6. The method according to claim 3, wherein the measuring in the step (c) is performed by quantitative RT-PCR using the nucleic acid(s) as primer(s).

7. The method according to claim 3, wherein the measuring in the step (c) is performed by hybridization using the nucleic acid(s) as probe(s).


SEQUENCE LISTING (1000 sequences)

SEQ ID NO: 1 - RNA, 22 nt, Homo sapiens
  source 1..22: mol_type = transcribed RNA
tggcgggggtagagctggctgc

SEQ ID NO: 2 - RNA, 20 nt, Homo sapiens
  source 1..20: mol_type = transcribed RNA
ggggagctgtggaagcagta

SEQ ID NO: 3 - RNA, 22 nt, Homo sapiens
  source 1..22: mol_type = transcribed RNA
caggcacgggagctcaggtgag

SEQ ID NO: 4 - RNA, 22 nt, Homo sapiens
  source 1..22: mol_type = transcribed RNA
atatacagggggagactcttat

SEQ ID NO: 5 - RNA, 19 nt, Homo sapiens
  source 1..19: mol_type = transcribed RNA
ggcttgcatgggggactgg

SEQ ID NO: 6 - RNA, 20 nt, Homo sapiens
  source 1..20: mol_type = transcribed RNA
gcggagagagaatggggagc

SEQ ID NO: 7 - RNA, 20 nt, Homo sapiens
  source 1..20: mol_type = transcribed RNA
gtgagtcagggtggggctgg

SEQ ID NO: 8 - RNA, 21 nt, Homo sapiens
  source 1..21: mol_type = transcribed RNA
ccgtcgccgccacccgagccg

SEQ ID NO: 9 - RNA, 22 nt, Homo sapiens
  source 1..22: mol_type = transcribed RNA
atatacagggggagactctcat

SEQ ID NO: 10 - RNA, 23 nt, Homo sapiens
  source 1..23: mol_type = transcribed RNA
gggatggtagaccggtgacgtgc

SEQ ID NO: 11 - RNA, 21 nt, Homo sapiens
  source 1..21: mol_type = transcribed RNA
tggcagggaggctgggagggg

SEQ ID NO: 12 - RNA, 23 nt, Homo sapiens
  source 1..23: mol_type = transcribed RNA
gtgagtgggagccccagtgtgtg

SEQ ID NO: 13 - RNA, 19 nt, Homo sapiens
  source 1..19: mol_type = transcribed RNA
aatggatttttggagcagg

SEQ ID NO: 14 - RNA, 24 nt, Homo sapiens
  source 1..24: mol_type = transcribed RNA
aggagggaggagatgggccaagtt

SEQ ID NO: 15 - RNA, 20 nt, Homo sapiens
  source 1..20: mol_type = transcribed RNA
tcgcgccccggctcccgttc

SEQ ID NO: 16 - RNA, 22 nt, Homo sapiens
  source 1..22: mol_type = transcribed RNA
ctcggcgcggggcgcgggctcc

SEQ ID NO: 17 - RNA, 21 nt, Homo sapiens
  source 1..21: mol_type = transcribed RNA
gccctccgcccgtgcaccccg

SEQ ID NO: 18 - RNA, 23 nt, Homo sapiens
  source 1..23: mol_type = transcribed RNA
cgggtagagagggcagtgggagg

SEQ ID NO: 19 - RNA, 22 nt, Homo sapiens
  source 1..22: mol_type = transcribed RNA
gagcttttggcccgggttatac

SEQ ID NO: 20 - RNA, 22 nt, Homo sapiens
  source 1..22: mol_type = transcribed RNA
ttggggaaacggccgctgagtg

SEQ ID NO: 21 - RNA, 21 nt, Homo sapiens
  source 1..21: mol_type = transcribed RNA
gcagggacagcaaaggggtgc

SEQ ID NO: 22 - RNA, 22 nt, Homo sapiens
  source 1..22: mol_type = transcribed RNA
agcagaggcagagaggctcagg

SEQ ID NO: 23 - RNA, 22 nt, Homo sapiens
  source 1..22: mol_type = transcribed RNA
gttgggacaagaggacggtctt

SEQ ID NO: 24 - RNA, 19 nt, Homo sapiens
  source 1..19: mol_type = transcribed RNA
gagggcgggtggaggagga

SEQ ID NO: 25 - RNA, 22 nt, Homo sapiens
  source 1..22: mol_type = transcribed RNA
aaagatctggaagtgggagaca

SEQ ID NO: 26 - RNA, 21 nt, Homo sapiens
  source 1..21: mol_type = transcribed RNA
cctgcagagaggaagcccttc

SEQ ID NO: 27 - RNA, 22 nt, Homo sapiens
  source 1..22: mol_type = transcribed RNA
ggctttctagtctcagctctcc

SEQ ID NO: 28 - RNA, 22 nt, Homo sapiens
  source 1..22: mol_type = transcribed RNA
tggggcggagcttccggaggcc

SEQ ID NO: 29 - RNA, 23 nt, Homo sapiens
  source 1..23: mol_type = transcribed RNA
tggggacgtagctggccagacag

SEQ ID NO: 30 - RNA, 22 nt, Homo sapiens
  source 1..22: mol_type = transcribed RNA
agctctgctgctcactggcagt

SEQ ID NO: 31 - RNA, 22 nt, Homo sapiens
  source 1..22: mol_type = transcribed RNA
aaaagctgggttgagagggcaa

SEQ ID NO: 32 - RNA, 23 nt, Homo sapiens
  source 1..23: mol_type = transcribed RNA
gggggggcaggaggggctcaggg

SEQ ID NO: 33 - RNA, 20 nt, Homo sapiens
  source 1..20: mol_type = transcribed RNA
gaatcggaaaggaggcgccg

SEQ ID NO: 34 - RNA, 22 nt, Homo sapiens
  source 1..22: mol_type = transcribed RNA
gggaccatcctgcctgctgtgg

SEQ ID NO: 35 - RNA, 22 nt, Homo sapiens
  source 1..22: mol_type = transcribed RNA
gtgggctgggctgggctgggcc

SEQ ID NO: 36 - RNA, 22 nt, Homo sapiens
  source 1..22: mol_type = transcribed RNA
gcctgctggggtggaacctggt

SEQ ID NO: 37 - RNA, 22 nt, Homo sapiens
  source 1..22: mol_type = transcribed RNA
actcaaaatgggggcgctttcc

SEQ ID NO: 38 - RNA, 20 nt, Homo sapiens
  source 1..20: mol_type = transcribed RNA
gctcggactgagcaggtggg

SEQ ID NO: 39 - RNA, 23 nt, Homo sapiens
  source 1..23: mol_type = transcribed RNA
acaggcggctgtagcaatggggg

SEQ ID NO: 40 - RNA, 22 nt, Homo sapiens
  source 1..22: mol_type = transcribed RNA
cagttgggtctaggggtcagga

SEQ ID NO: 41 - RNA, 18 nt, Homo sapiens
  source 1..18: mol_type = transcribed RNA
gggtcccggggagggggg

SEQ ID NO: 42 - RNA, 17 nt, Homo sapiens
  source 1..17: mol_type = transcribed RNA
gggagtctacagcaggg

SEQ ID NO: 43 - RNA, 18 nt, Homo sapiens
  source 1..18: mol_type = transcribed RNA
gaggctgaaggaagatgg

SEQ ID NO: 44 - RNA, 22 nt, Homo sapiens
  source 1..22: mol_type = transcribed RNA
caaggagacgggaacatggagc

SEQ ID NO: 45 - RNA, 20 nt, Homo sapiens
  source 1..20: mol_type = transcribed RNA
aaaagctgggctgagaggcg

SEQ ID NO: 46 - RNA, 21 nt, Homo sapiens
  source 1..21: mol_type = transcribed RNA
acaggagtgggggtgggacat

SEQ ID NO: 47 - RNA, 17 nt, Homo sapiens
  source 1..17: mol_type = transcribed RNA
ggtgggggctgttgttt

SEQ ID NO: 48 - RNA, 22 nt, Homo sapiens
  source 1..22: mol_type = transcribed RNA
cgtcccggggctgcgcgaggca

SEQ ID NO: 49 - RNA, 22 nt, Homo sapiens
  source 1..22: mol_type = transcribed RNA
ccaggaggcggaggaggtggag

SEQ ID NO: 50 - RNA, 21 nt, Homo sapiens
  source 1..21: mol_type = transcribed RNA
agccaagtggaagttacttta

SEQ ID NO: 51 - RNA, 16 nt, Homo sapiens
  source 1..16: mol_type = transcribed RNA
accgcctgcccagtga

SEQ ID NO: 52 - RNA, 17 nt, Homo sapiens
  source 1..17: mol_type = transcribed RNA
gctgggcgaggctggca

SEQ ID NO: 53 - RNA, 18 nt, Homo sapiens
  source 1..18: mol_type = transcribed RNA
agggggcgggctccggcg

SEQ ID NO: 54 - RNA, 21 nt, Homo sapiens
  source 1..21: mol_type = transcribed RNA
tggggctagtgatgcaggacg

SEQ ID NO: 55 - RNA, 18 nt, Homo sapiens
  source 1..18: mol_type = transcribed RNA
aggctgggctgggacgga

SEQ ID NO: 56 - RNA, 22 nt, Homo sapiens
  source 1..22: mol_type = transcribed RNA
agactgacggctggaggcccat

SEQ ID NO: 57 - RNA, 22 nt, Homo sapiens
  source 1..22: mol_type = transcribed RNA
aggactggactcccggcagccc

SEQ ID NO: 58 - RNA, 18 nt, Homo sapiens
  source 1..18: mol_type = transcribed RNA
cccagcaggacgggagcg

SEQ ID NO: 59 - RNA, 17 nt, Homo sapiens
  source 1..17: mol_type = transcribed RNA
gtggacctggctgggac

SEQ ID NO: 60 - RNA, 21 nt, Homo sapiens
  source 1..21: mol_type = transcribed RNA
tcttgaagtcagaacccgcaa

SEQ ID NO: 61 - RNA, 23 nt, Homo sapiens
  source 1..23: mol_type = transcribed RNA
tgggccagggagcagctggtggg

SEQ ID NO: 62 - RNA, 22 nt, Homo sapiens
  source 1..22: mol_type = transcribed RNA
actgggaagaggagctgaggga

SEQ ID NO: 63 - RNA, 23 nt, Homo sapiens
  source 1..23: mol_type = transcribed RNA
tgggctgagggcaggaggcctgt

SEQ ID NO: 64 - RNA, 24 nt, Homo sapiens
  source 1..24: mol_type = transcribed RNA
agctgagctccatggacgtgcagt

SEQ ID NO: 65 - RNA, 23 nt, Homo sapiens
  source 1..23: mol_type = transcribed RNA
ctgggggacgcgtgagcgcgagc

SEQ ID NO: 66 - RNA, 25 nt, Homo sapiens
  source 1..25: mol_type = transcribed RNA
agcggggaggaagtgggcgctgctt

SEQ ID NO: 67 - RNA, 23 nt, Homo sapiens
  source 1..23: mol_type = transcribed RNA
gccccggcgcgggcgggttctgg

SEQ ID NO: 68 - RNA, 22 nt, Homo sapiens
  source 1..22: mol_type = transcribed RNA
agcaaggcggcatctctctgat

SEQ ID NO: 69 - RNA, 18 nt, Homo sapiens
  source 1..18: mol_type = transcribed RNA
gggtgagggcaggtggtt

SEQ ID NO: 70 - RNA, 21 nt, Homo sapiens
  source 1..21: mol_type = transcribed RNA
agctgtacctgaaaccaagca

SEQ ID NO: 71 - RNA, 23 nt, Homo sapiens
  source 1..23: mol_type = transcribed RNA
ggcaggagggctgtgccaggttg

SEQ ID NO: 72 - RNA, 22 nt, Homo sapiens
  source 1..22: mol_type = transcribed RNA
atagtgggaagctggcagattc

SEQ ID NO: 73 - RNA, 23 nt, Homo sapiens
  source 1..23: mol_type = transcribed RNA
ctggcggagcccattccatgcca

SEQ ID NO: 74 - RNA, 22 nt, Homo sapiens
  source 1..22: mol_type = transcribed RNA
gctgcgggctgcggtcagggcg

SEQ ID NO: 75 - RNA, 22 nt, Homo sapiens
  source 1..22: mol_type = transcribed RNA
aggactgatcctctcgggcagg

SEQ ID NO: 76 - RNA, 22 nt, Homo sapiens
  source 1..22: mol_type = transcribed RNA
aaggcccgggctttcctcccag

SEQ ID NO: 77 - RNA, 22 nt, Homo sapiens
  source 1..22: mol_type = transcribed RNA
tgcggggacaggccagggcatc

SEQ ID NO: 78 - RNA, 22 nt, Homo sapiens
  source 1..22: mol_type = transcribed RNA
agccaggctctgaagggaaagt

SEQ ID NO: 79 - RNA, 21 nt, Homo sapiens
  source 1..21: mol_type = transcribed RNA
cgcctgcccagccctcctgct

SEQ ID NO: 80 - RNA, 24 nt, Homo sapiens
  source 1..24: mol_type = transcribed RNA
gatgcgccgcccactgccccgcgc

SEQ ID NO: 81 - RNA, 22 nt, Homo sapiens
  source 1..22: mol_type = transcribed RNA
tgaggcccttggggcacagtgg

SEQ ID NO: 82 - RNA, 22 nt, Homo sapiens
  source 1..22: mol_type = transcribed RNA
agggggatggcagagcaaaatt

SEQ ID NO: 83 - RNA, 21 nt, Homo sapiens
  source 1..21: mol_type = transcribed RNA
gggagtgcagggcagggtttc

SEQ ID NO: 84 - RNA, 23 nt, Homo sapiens
  source 1..23: mol_type = transcribed RNA
ccggggcagattggtgtagggtg

SEQ ID NO: 85 - RNA, 22 nt, Homo sapiens
  source 1..22: mol_type = transcribed RNA
ttcagatcccagcggtgcctct

SEQ ID NO: 86 - RNA, 22 nt, Homo sapiens
  source 1..22: mol_type = transcribed RNA
agggaaggggacgagggttggg

SEQ ID NO: 87 - RNA, 22 nt, Homo sapiens
  source 1..22: mol_type = transcribed RNA
gaaatcaagcgtgggtgagacc

SEQ ID NO: 88 - RNA, 23 nt, Homo sapiens
  source 1..23: mol_type = transcribed RNA
gtttgcacgggtgggccttgtct

SEQ ID NO: 89 - RNA, 20 nt, Homo sapiens
  source 1..20: mol_type = transcribed RNA
gggctggggcgcggggaggt

SEQ ID NO: 90 - RNA, 19 nt, Homo sapiens
  source 1..19: mol_type = transcribed RNA
ggggagcgaggggcggggc

SEQ ID NO: 91 - RNA, 20 nt, Homo sapiens
  source 1..20: mol_type = transcribed RNA
gggaaaaggaagggggagga

SEQ ID NO: 92 - RNA, 19 nt, Homo sapiens
  source 1..19: mol_type = transcribed RNA
agcagggctggggattgca

SEQ ID NO: 93 - RNA, 22 nt, Homo sapiens
  source 1..22: mol_type = transcribed RNA
cagcaggggagagagaggagtc

SEQ ID NO: 94 - RNA, 24 nt, Homo sapiens
  source 1..24: mol_type = transcribed RNA
ctgcaggcagaagtggggctgaca

SEQ ID NO: 95 - RNA, 20 nt, Homo sapiens
  source 1..20: mol_type = transcribed RNA
tctcttcatctaccccccag

SEQ ID NO: 96 - RNA, 22 nt, Homo sapiens
  source 1..22: mol_type = transcribed RNA
tggtgggccgcagaacatgtgc

SEQ ID NO: 97 - RNA, 25 nt, Homo sapiens
  source 1..25: mol_type = transcribed RNA
ggcggagggaagtaggtccgttggt

SEQ ID NO: 98 - RNA, 19 nt, Homo sapiens
  source 1..19: mol_type = transcribed RNA
tgcgcctcgggtgagcatg

SEQ ID NO: 99 - RNA, 20 nt, Homo sapiens
  source 1..20: mol_type = transcribed RNA
aggcgcacccgaccacatgc

SEQ ID NO: 100 - RNA, 23 nt, Homo sapiens
  source 1..23: mol_type = transcribed RNA
ctgggcccgcggcgggcgtgggg

SEQ ID NO: 101 - RNA, 21 nt, Homo sapiens
  source 1..21: mol_type = transcribed RNA
tcatccccctcgccctctcag

SEQ ID NO: 102 - RNA, 21 nt, Homo sapiens
  source 1..21: mol_type = transcribed RNA
ttggggtggtcggccctggag

SEQ ID NO: 103 - RNA, 23 nt, Homo sapiens
  source 1..23: mol_type = transcribed RNA
agggtggggctggaggtggggct

SEQ ID NO: 104 - RNA, 23 nt, Homo sapiens
  source 1..23: mol_type = transcribed RNA
cggggccatggagcagcctgtgt

SEQ ID NO: 105 - RNA, 21 nt, Homo sapiens
  source 1..21: mol_type = transcribed RNA
ctccccggcctctgcccccag

SEQ ID NO: 106 - RNA, 24 nt, Homo sapiens
  source 1..24: mol_type = transcribed RNA
cgggtgggagcagatcttattgag

SEQ ID NO: 107 - RNA, 21 nt, Homo sapiens
  source 1..21: mol_type = transcribed RNA
aggtgggtatggaggagccct

SEQ ID NO: 108 - RNA, 22 nt, Homo sapiens
  source 1..22: mol_type = transcribed RNA
ctcgggagggcatgggccaggc

SEQ ID NO: 109 - RNA, 21 nt, Homo sapiens
  source 1..21: mol_type = transcribed RNA
gcggtggggccggaggggcgt

SEQ ID NO: 110 - RNA, 24 nt, Homo sapiens
  source 1..24: mol_type = transcribed RNA
gtaggggcgtcccgggcgcgcggg

SEQ ID NO: 111 - RNA, 20 nt, Homo sapiens
  source 1..20: mol_type = transcribed RNA
cagggggactgggggtgagc

SEQ ID NO: 112 - RNA, 21 nt, Homo sapiens
  source 1..21: mol_type = transcribed RNA
gaagctctcccctccccgcag

SEQ ID NO: 113 - RNA, 25 nt, Homo sapiens
  source 1..25: mol_type = transcribed RNA
aggagggaaggggctgagaacagga

SEQ ID NO: 114 - RNA, 21 nt, Homo sapiens
  source 1..21: mol_type = transcribed RNA
cacctctcctggcatcgcccc

SEQ ID NO: 115 - RNA, 20 nt, Homo sapiens
  source 1..20: mol_type = transcribed RNA
ctaggtggggggcttgaagc

SEQ ID NO: 116 - RNA, 22 nt, Homo sapiens
  source 1..22: mol_type = transcribed RNA
ctgggggtggggggctgggcgt

SEQ ID NO: 117 - RNA, 23 nt, Homo sapiens
  source 1..23: mol_type = transcribed RNA
ttgctctgctcccccgcccccag

SEQ ID NO: 118 - RNA, 22 nt, Homo sapiens
  source 1..22: mol_type = transcribed RNA
tagggggcggcttgtggagtgt

SEQ ID NO: 119 - RNA, 22 nt, Homo sapiens
  source 1..22: mol_type = transcribed RNA
gtgagccagtggaatggagagg

SEQ ID NO: 120 - RNA, 25 nt, Homo sapiens
  source 1..25: mol_type = transcribed RNA
atggggtgagatggggaggagcagc

SEQ ID NO: 121 - RNA, 22 nt, Homo sapiens
  source 1..22: mol_type = transcribed RNA
ttggggtggagggccaaggagc

SEQ ID NO: 122 - RNA, 20 nt, Homo sapiens
  source 1..20: mol_type = transcribed RNA
cagggaaccagttggggctt

SEQ ID NO: 123 - RNA, 22 nt, Homo sapiens
  source 1..22: mol_type = transcribed RNA
gtaggggaggttgggccaggga

SEQ ID NO: 124 - RNA, 23 nt, Homo sapiens
  source 1..23: mol_type = transcribed RNA
tcaataggaaagaggtgggacct

SEQ ID NO: 125 - RNA, 22 nt, Homo sapiens
  source 1..22: mol_type = transcribed RNA
gtgcggaacgctggccggggcg

SEQ ID NO: 126 - RNA, 22 nt, Homo sapiens
  source 1..22: mol_type = transcribed RNA
gtgaggaggggctggcagggac

SEQ ID NO: 127 - RNA, 22 nt, Homo sapiens
  source 1..22: mol_type = transcribed RNA
actgggtaggtggggctccagg

SEQ ID NO: 128 - RNA, 21 nt, Homo sapiens
  source 1..21: mol_type = transcribed RNA
ccgccttctctcctcccccag

SEQ ID NO: 129 - RNA, 22 nt, Homo sapiens
  source 1..22: mol_type = transcribed RNA
tcggcctggggaggaggaaggg

SEQ ID NO: 130 - RNA, 21 nt, Homo sapiens
  source 1..21: mol_type = transcribed RNA
ctggggggaggagaccctgct

SEQ ID NO: 131 - RNA, 21 nt, Homo sapiens
  source 1..21: mol_type = transcribed RNA
tctgtggagtggggtgcctgt

SEQ ID NO: 132 - RNA, 19 nt, Homo sapiens
  source 1..19: mol_type = transcribed RNA
cggggtcggcggcgacgtg

SEQ ID NO: 133 - RNA, 21 nt, Homo sapiens
  source 1..21: mol_type = transcribed RNA
cagcggagcctggagagaagg

SEQ ID NO: 134 - RNA, 22 nt, Homo sapiens
  source 1..22: mol_type = transcribed RNA
cgggactgtagagggcatgagc

SEQ ID NO: 135 - RNA, 24 nt, Homo sapiens
  source 1..24: mol_type = transcribed RNA
ccatgaagcagtgggtaggaggac

SEQ ID NO: 136 - RNA, 20 nt, Homo sapiens
  source 1..20: mol_type = transcribed RNA
cggtggactggagtgggtgg

SEQ ID NO: 137 - RNA, 22 nt, Homo sapiens
  source 1..22: mol_type = transcribed RNA
acctggcagcagggagcgtcgt

SEQ ID NO: 138 - RNA, 23 nt, Homo sapiens
  source 1..23: mol_type = transcribed RNA
cggccccacgcaccagggtaaga

SEQ ID NO: 139 - RNA, 21 nt, Homo sapiens
  source 1..21: mol_type = transcribed RNA
gctgggaaggcaaagggacgt

SEQ ID NO: 140 - RNA, 22 nt, Homo sapiens
  source 1..22: mol_type = transcribed RNA
cagaaggggagttgggagcaga

SEQ ID NO: 141 - RNA, 20 nt, Homo sapiens
  source 1..20: mol_type = transcribed RNA
ggcggcggcggaggcggggg

SEQ ID NO: 142 - RNA, 21 nt, Homo sapiens
  source 1..21: mol_type = transcribed RNA
cgtcccaccccccactcctgt

SEQ ID NO: 143 - RNA, 17 nt, Homo sapiens
  source 1..17: mol_type = transcribed RNA
agggtgtgtgtgttttt

SEQ ID NO: 144 - RNA, 23 nt, Homo sapiens
  source 1..23: mol_type = transcribed RNA
tgacacggagggtggcttgggaa

SEQ ID NO: 145 - RNA, 22 nt, Homo sapiens
  source 1..22: mol_type = transcribed RNA
caggaaggatttagggacaggc

SEQ ID NO: 146 - RNA, 17 nt, Homo sapiens
  source 1..17: mol_type = transcribed RNA
gcggggctgggcgcgcg

SEQ ID NO: 147 - RNA, 21 nt, Homo sapiens
  source 1..21: mol_type = transcribed RNA
tggctgttggagggggcaggc

SEQ ID NO: 148 - RNA, 22 nt, Homo sapiens
  source 1..22: mol_type = transcribed RNA
cagccctcctcccgcacccaaa

SEQ ID NO: 149 - RNA, 23 nt, Homo sapiens
  source 1..23: mol_type = transcribed RNA
tgtagagcagggagcaggaagct

SEQ ID NO: 150 - RNA, 21 nt, Homo sapiens
  source 1..21: mol_type = transcribed RNA
agcagacttgacctacaatta

SEQ ID NO: 151 - RNA, 22 nt, Homo sapiens
  source 1..22: mol_type = transcribed RNA
agacacatttggagagggaacc

SEQ ID NO: 152 - RNA, 20 nt, Homo sapiens
  source 1..20: mol_type = transcribed RNA
tagggggtggcaggctggcc

SEQ ID NO: 153 - RNA, 23 nt, Homo sapiens
  source 1..23: mol_type = transcribed RNA
cagggagaaggtggaagtgcaga

SEQ ID NO: 154 - RNA, 20 nt, Homo sapiens
  source 1..20: mol_type = transcribed RNA
ggggaggtgtgcagggctgg

SEQ ID NO: 155 - RNA, 19 nt, Homo sapiens
  source 1..19: mol_type = transcribed RNA
tgcggcagagctggggtca

SEQ ID NO: 156 - RNA, 23 nt, Homo sapiens
  source 1..23: mol_type = transcribed RNA
gtgcgtggtggctcgaggcgggg

SEQ ID NO: 157 - RNA, 20 nt, Homo sapiens
  source 1..20: mol_type = transcribed RNA
tgggctgctgagaaggggca

SEQ ID NO: 158 - RNA, 21 nt, Homo sapiens
  source 1..21: mol_type = transcribed RNA
caggcaggtgtagggtggagc

SEQ ID NO: 159 - RNA, 20 nt, Homo sapiens
  source 1..20: mol_type = transcribed RNA
acccgcccgtctccccacag

SEQ ID NO: 160 - RNA, 22 nt, Homo sapiens
  source 1..22: mol_type = transcribed RNA
tgggggaggaaggacaggccat

SEQ ID NO: 161 - RNA, 24 nt, Homo sapiens
  source 1..24: mol_type = transcribed RNA
cctggggacaggggattggggcag

SEQ ID NO: 162 - RNA, 22 nt, Homo sapiens
  source 1..22: mol_type = transcribed RNA
aggcagcggggtgtagtggata

SEQ ID NO: 163 - RNA, 22 nt, Homo sapiens
  source 1..22: mol_type = transcribed RNA
tattgcactcgtcccggcctcc

SEQ ID NO: 164 - RNA, 22 nt, Homo sapiens
  source 1..22: mol_type = transcribed RNA
ctcctggggcccgcactctcgc

SEQ ID NO: 165 - RNA, 22 nt, Homo sapiens
  source 1..22: mol_type = transcribed RNA
ccgggagaaggaggtggcctgg

SEQ ID NO: 166 - RNA, 22 nt, Homo sapiens
  source 1..22: mol_type = transcribed RNA
actggacttagggtcagaaggc

SEQ ID NO: 167 - RNA, 22 nt, Homo sapiens
  source 1..22: mol_type = transcribed RNA
ggctacaacacaggacccgggc

SEQ ID NO: 168 - RNA, 23 nt, Homo sapiens
  source 1..23: mol_type = transcribed RNA
gagggcagcgtgggtgtggcgga

SEQ ID NO: 169 - RNA, 22 nt, Homo sapiens
  source 1..22: mol_type = transcribed RNA
cccctggggctgggcaggcgga

SEQ ID NO: 170 - RNA, 23 nt, Homo sapiens
  source 1..23: mol_type = transcribed RNA
agcagcattgtacagggctatga

SEQ ID NO: 171 - RNA, 23 nt, Homo sapiens
  source 1..23: mol_type = transcribed RNA
agcagcattgtacagggctatca

SEQ ID NO: 172 - RNA, 20 nt, Homo sapiens
  source 1..20: mol_type = transcribed RNA
cctgagcccgggccgcgcag

SEQ ID NO: 173 - RNA, 22 nt, Homo sapiens
  source 1..22: mol_type = transcribed RNA
tgagcccctgtgccgcccccag

SEQ ID NO: 174 - RNA, 22 nt, Homo sapiens
  source 1..22: mol_type = transcribed RNA
gtgggtacggcccagtgggggg

SEQ ID NO: 175 - RNA, 21 nt, Homo sapiens
  source 1..21: mol_type = transcribed RNA
gtgggcgggggcaggtgtgtg

SEQ ID NO: 176 - RNA, 24 nt, Homo sapiens
  source 1..24: mol_type = transcribed RNA
gtgggtagggtttgggggagagcg

SEQ ID NO: 177 - RNA, 22 nt, Homo sapiens
  source 1..22: mol_type = transcribed RNA
agtgggaggccagggcacggca

SEQ ID NO: 178 - RNA, 21 nt, Homo sapiens
  source 1..21: mol_type = transcribed RNA
cgggggcggggccgaagcgcg

SEQ ID NO: 179 - RNA, 24 nt, Homo sapiens
  source 1..24: mol_type = transcribed RNA
ccccgggaacgtcgagactggagc

SEQ ID NO: 180 - RNA, 22 nt, Homo sapiens
  source 1..22: mol_type = transcribed RNA
acgcccttcccccccttcttca

SEQ ID NO: 181 - RNA, 24 nt, Homo sapiens
  source 1..24: mol_type = transcribed RNA
agcctggaagctggagcctgcagt

SEQ ID NO: 182 - RNA, 19 nt, Homo sapiens
  source 1..19: mol_type = transcribed RNA
atcccaccactgccaccat

SEQ ID NO: 183 - RNA, 18 nt, Homo sapiens
  source 1..18: mol_type = transcribed RNA
cgggcgtggtggtggggg

SEQ ID NO: 184 - RNA, 20 nt, Homo sapiens
  source 1..20: mol_type = transcribed RNA
cgggcgtggtggtgggggtg

SEQ ID NO: 185 - RNA, 21 nt, Homo sapiens
  source 1..21: mol_type = transcribed RNA
accactgcactccagcctgag

SEQ ID NO: 186 - RNA, 23 nt, Homo sapiens
  source 1..23: mol_type = transcribed RNA
cggggccgtagcactgtctgaga

SEQ ID NO: 187 - RNA, 23 nt, Homo sapiens
  source 1..23: mol_type = transcribed RNA
gggggccgatacactgtacgaga

SEQ ID NO: 188 - RNA, 19 nt, Homo sapiens
  source 1..19: mol_type = transcribed RNA
tggatttttggatcaggga

SEQ ID NO: 189 - RNA, 22 nt, Homo sapiens
  source 1..22: mol_type = transcribed RNA
ctggtacaggcctgggggacag

SEQ ID NO: 190 - RNA, 22 nt, Homo sapiens
  source 1..22: mol_type = transcribed RNA
actgcagtgaaggcacttgtag

SEQ ID NO: 191 - RNA, 21 nt, Homo sapiens
  source 1..21: mol_type = transcribed RNA
cggcggggacggcgattggtc

SEQ ID NO: 192 - RNA, 22 nt, Homo sapiens
  source 1..22: mol_type = transcribed RNA
cgcaggggccgggtgctcaccg

SEQ ID NO: 193 - RNA, 22 nt, Homo sapiens
  source 1..22: mol_type = transcribed RNA
ggaggggtcccgcactgggagg

SEQ ID NO: 194 - RNA, 20 nt, Homo sapiens
  source 1..20: mol_type = transcribed RNA
ccccagggcgacgcggcggg

SEQ ID NO: 195 - RNA, 23 nt, Homo sapiens
  source 1..23: mol_type = transcribed RNA
caacggaatcccaaaagcagctg

SEQ ID NO: 196 - RNA, 22 nt, Homo sapiens
  source 1..22: mol_type = transcribed RNA
aagctgccagttgaagaactgt

SEQ ID NO: 197 - RNA, 21 nt, Homo sapiens
  source 1..21: mol_type = transcribed RNA
atcacattgccagggattacc

SEQ ID NO: 198 - RNA, 22 nt, Homo sapiens
  source 1..22: mol_type = transcribed RNA
tggctcagttcagcaggaacag

SEQ ID NO: 199 - RNA, 22 nt, Homo sapiens
  source 1..22: mol_type = transcribed RNA
gagggttgggtggaggctctcc

SEQ ID NO: 200 - RNA, 21 nt, Homo sapiens
  source 1..21: mol_type = transcribed RNA
agggccccccctcaatcctgt

SEQ ID NO: 201 - RNA, 23 nt, Homo sapiens
  source 1..23: mol_type = transcribed RNA
tcgaggactggtggaagggcctt

SEQ ID NO: 202 - RNA, 23 nt, Homo sapiens
  source 1..23: mol_type = transcribed RNA
ttagggagtagaagggtggggag

SEQ ID NO: 203 - RNA, 23 nt, Homo sapiens
  source 1..23: mol_type = transcribed RNA
agaggctttgtgcggatacgggg

SEQ ID NO: 204 - RNA, 18 nt, Homo sapiens
  source 1..18: mol_type = transcribed RNA
cggggcggcaggggcctc

SEQ ID NO: 205 - RNA, 23 nt, Homo sapiens
  source 1..23: mol_type = transcribed RNA
ggaggcgcaggctcggaaaggcg

SEQ ID NO: 206 - RNA, 22 nt, Homo sapiens
  source 1..22: mol_type = transcribed RNA
aaaagctgggttgagagggcga

SEQ ID NO: 207 - RNA, 21 nt, Homo sapiens
  source 1..21: mol_type = transcribed RNA
aggggtgctatctgtgattga

SEQ ID NO: 208 - RNA, 20 nt, Homo sapiens
  source 1..20: mol_type = transcribed RNA
cgcgggtcggggtctgcagg

SEQ ID NO: 209 - RNA, 21 nt, Homo sapiens
  source 1..21: mol_type = transcribed RNA
agccgcggggatcgccgaggg

SEQ ID NO: 210 - RNA, 17 nt, Homo sapiens
  source 1..17: mol_type = transcribed RNA
ggcgggtgcgggggtgg

SEQ ID NO: 211 - RNA, 23 nt, Homo sapiens
  source 1..23: mol_type = transcribed RNA
agggacttttgggggcagatgtg

SEQ ID NO: 212 - RNA, 18 nt, Homo sapiens
  source 1..18: mol_type = transcribed RNA
agcaggtgcggggcggcg

SEQ ID NO: 213 - RNA, 23 nt, Homo sapiens
  source 1..23: mol_type = transcribed RNA
tgaggatatggcagggaagggga

SEQ ID NO: 214 - RNA, 20 nt, Homo sapiens
  source 1..20: mol_type = transcribed RNA
actcaaactgtgggggcact

SEQ ID NO: 215 - RNA, 20 nt, Homo sapiens
  source 1..20: mol_type = transcribed RNA
gtgggttggggcgggctctg

SEQ ID NO: 216 - RNA, 23 nt, Homo sapiens
  source 1..23: mol_type = transcribed RNA
tgaggggcagagagcgagacttt

SEQ ID NO: 217 - RNA, 18 nt, Homo sapiens
  source 1..18: mol_type = transcribed RNA
ccagaggtggggactgag

SEQ ID NO: 218 - RNA, 20 nt, Homo sapiens
  source 1..20: mol_type = transcribed RNA
tcagggagtcaggggagggc

SEQ ID NO: 219 - RNA, 19 nt, Homo sapiens
  source 1..19: mol_type = transcribed RNA
gggggaagaaaaggtgggg

SEQ ID NO: 220 - RNA, 17 nt, Homo sapiens
  source 1..17: mol_type = transcribed RNA
accccactcctggtacc

SEQ ID NO: 221 - RNA, 22 nt, Homo sapiens
  source 1..22: mol_type = transcribed RNA
ctgggacaggaggaggaggcag

SEQ ID NO: 222 - RNA, 18 nt, Homo sapiens
  source 1..18: mol_type = transcribed RNA
ggtgggcttcccggaggg

SEQ ID NO: 223 - RNA, 17 nt, Homo sapiens
  source 1..17: mol_type = transcribed RNA
gccggacaagagggagg

SEQ ID NO: 224 - RNA, 22 nt, Homo sapiens
  source 1..22: mol_type = transcribed RNA
cagggctggcagtgacatgggt

SEQ ID NO: 225 - RNA, 20 nt, Homo sapiens
  source 1..20: mol_type = transcribed RNA
ggctccttggtctaggggta

SEQ ID NO: 226 - RNA, 20 nt, Homo sapiens
  source 1..20: mol_type = transcribed RNA
ggatccgagtcacggcacca

SEQ ID NO: 227 - RNA, 22 nt, Homo sapiens
  source 1..22: mol_type = transcribed RNA
tggcggcggtagttatgggctt

SEQ ID NO: 228 - RNA, 18 nt, Homo sapiens
  source 1..18: mol_type = transcribed RNA
ggtggggggtgttgtttt

SEQ ID NO: 229 - RNA, 20 nt, Homo sapiens
  source 1..20: mol_type = transcribed RNA
ctgggttgggctgggctggg

SEQ ID NO: 230 - RNA, 17 nt, Homo sapiens
  source 1..17: mol_type = transcribed RNA
gggagaagggtcggggc

SEQ ID NO: 231 - RNA, 22 nt, Homo sapiens
  source 1..22: mol_type = transcribed RNA
aaaccgttaccattactgagtt

SEQ ID NO: 232 - RNA, 24 nt, Homo sapiens
  source 1..24: mol_type = transcribed RNA
tgggcgaggggtgggctctcagag

SEQ ID NO: 233 - RNA, 20 nt, Homo sapiens
  source 1..20: mol_type = transcribed RNA
cggggtgggtgaggtcgggc

SEQ ID NO: 234 - RNA, 26 nt, Homo sapiens
  source 1..26: mol_type = transcribed RNA
ctcggccgcggcgcgtagcccccgcc

SEQ ID NO: 235 - RNA, 21 nt, Homo sapiens
  source 1..21: mol_type = transcribed RNA
ctgggctcgggacgcgcggct

SEQ ID NO: 236 - RNA, 22 nt, Homo sapiens
  source 1..22: mol_type = transcribed RNA
ggggctgtgattgaccagcagg

SEQ ID NO: 237 - RNA, 22 nt, Homo sapiens
  source 1..22: mol_type = transcribed RNA
ttgaggagacatggtgggggcc

SEQ ID NO: 238 - RNA, 22 nt, Homo sapiens
  source 1..22: mol_type = transcribed RNA
caggaggcagtgggcgagcagg

SEQ ID NO: 239 - RNA, 22 nt, Homo sapiens
  source 1..22: mol_type = transcribed RNA
agggggcgcagtcactgacgtg

SEQ ID NO: 240 - RNA, 22 nt, Homo sapiens
  source 1..22: mol_type = transcribed RNA
tggggaaggcgtcagtgtcggg

SEQ ID NO: 241 - RNA, 25 nt, Homo sapiens
  source 1..25: mol_type = transcribed RNA
aagggaggaggagcggaggggccct

SEQ ID NO: 242 - RNA, 23 nt, Homo sapiens
  source 1..23: mol_type = transcribed RNA
tgagtggggctcccgggacggcg

SEQ ID NO: 243 - RNA, 24 nt, Homo sapiens
  source 1..24: mol_type = transcribed RNA
aggcaggggctggtgctgggcggg

SEQ ID NO: 244 - RNA, 18 nt, Homo sapiens
  source 1..18: mol_type = transcribed RNA
cggtgagcgctcgctggc

SEQ ID NO: 245 - RNA, 21 nt, Homo sapiens
  source 1..21: mol_type = transcribed RNA
cggggcagctcagtacaggat

SEQ ID NO: 246 - RNA, 24 nt, Homo sapiens
  source 1..24: mol_type = transcribed RNA
agggctggactcagcggcggagct

SEQ ID NO: 247 - RNA, 21 nt, Homo sapiens
  source 1..21: mol_type = transcribed RNA
atccagttctctgagggggct

SEQ ID NO: 248 - RNA, 23 nt, Homo sapiens
  source 1..23: mol_type = transcribed RNA
agtgcctgagggagtaagagccc

SEQ ID NO: 249 - RNA, 22 nt, Homo sapiens
  source 1..22: mol_type = transcribed RNA
tgggggagtgcagtgattgtgg

SEQ ID NO: 250 - RNA, 21 nt, Homo sapiens
  source 1..21: mol_type = transcribed RNA
acggcccaggcggcattggtg

SEQ ID NO: 251 - RNA, 20 nt, Homo sapiens
  source 1..20: mol_type = transcribed RNA
agagatgaagcgggggggcg

SEQ ID NO: 252 - RNA, 24 nt, Homo sapiens
  source 1..24: mol_type = transcribed RNA
ggaggccggggtggggcggggcgg

SEQ ID NO: 253 - RNA, 20 nt, Homo sapiens
  source 1..20: mol_type = transcribed RNA
gcggaaggcggagcggcgga

SEQ ID NO: 254 - RNA, 18 nt, Homo sapiens
  source 1..18: mol_type = transcribed RNA
gtgaaggcccggcggaga

SEQ ID NO: 255 - RNA, 23 nt, Homo sapiens
  source 1..23: mol_type = transcribed RNA
gaacgcctgttcttgccaggtgg

SEQ ID NO: 256 - RNA, 22 nt, Homo sapiens
  source 1..22: mol_type = transcribed RNA
gggggtccccggtgctcggatc

SEQ ID NO: 257 - RNA, 22 nt, Homo sapiens
  source 1..22: mol_type = transcribed RNA
gctgggattacaggcatgagcc

SEQ ID NO: 258 - RNA, 25 nt, Homo sapiens
  source 1..25: mol_type = transcribed RNA
agggatcgcgggcgggtggcggcct

SEQ ID NO: 259 - RNA, 22 nt, Homo sapiens
  source 1..22: mol_type = transcribed RNA
agacacatttggagagggaccc

SEQ ID NO: 260 - RNA, 21 nt, Homo sapiens
  source 1..21: mol_type = transcribed RNA
aggaggcagcgctctcaggac

SEQ ID NO: 261 - RNA, 22 nt, Homo sapiens
  source 1..22: mol_type = transcribed RNA
aggcggggcgccgcgggaccgc

SEQ ID NO: 262 - RNA, 22 nt, Homo sapiens
  source 1..22: mol_type = transcribed RNA
ggtggcccggccgtgcctgagg

SEQ ID NO: 263 - RNA, 22 nt, Homo sapiens
  source 1..22: mol_type = transcribed RNA
aggcgatgtggggatgtagaga

SEQ ID NO: 264 - RNA, 23 nt, Homo sapiens
  source 1..23: mol_type = transcribed RNA
tgggcaggggcttattgtaggag

SEQ ID NO: 265 - RNA, 21 nt, Homo sapiens
  source 1..21: mol_type = transcribed RNA
cgggagctggggtctgcaggt

SEQ ID NO: 266 - RNA, 23 nt, Homo sapiens
  source 1..23: mol_type = transcribed RNA
ctcggggcaggcggctgggagcg

SEQ ID NO: 267 - RNA, 23 nt, Homo sapiens
  source 1..23: mol_type = transcribed RNA
cgaggggtagaagagcacagggg

SEQ ID NO: 268 - RNA, 22 nt, Homo sapiens
  source 1..22: mol_type = transcribed RNA
gtgggtgctggtgggagccgtg

SEQ ID NO: 269 - RNA, 22 nt, Homo sapiens
  source 1..22: mol_type = transcribed RNA
tcgggcctggggttgggggagc

SEQ ID NO: 270 - RNA, 22 nt, Homo sapiens
  source 1..22: mol_type = transcribed RNA
ggggggtgtggagccagggggc

SEQ ID NO: 271 - RNA, 23 nt, Homo sapiens
  source 1..23: mol_type = transcribed RNA
tggtgcggagagggcccacagtg

SEQ ID NO: 272 - RNA, 22 nt, Homo sapiens
  source 1..22: mol_type = transcribed RNA
tagggatgggaggccaggatga

SEQ ID NO: 273 - RNA, 19 nt, Homo sapiens
  source 1..19: mol_type = transcribed RNA
ctggggagtggctggggag

SEQ ID NO: 274 - RNA, 25 nt, Homo sapiens
  source 1..25: mol_type = transcribed RNA
gtgaggcggggccaggagggtgtgt

SEQ ID NO: 275 - RNA, 25 nt, Homo sapiens
  source 1..25: mol_type = transcribed RNA
tcggggcatgggggagggaggctgg

SEQ ID NO: 276 - RNA, 23 nt, Homo sapiens
  source 1..23: mol_type = transcribed RNA
tggggaaggcttggcagggaaga

SEQ ID NO: 277 - RNA, 25 nt, Homo sapiens
  source 1..25: mol_type = transcribed RNA
taggggtgggggaattcaggggtgt

SEQ ID NO: 278 - RNA, 20 nt, Homo sapiens
  source 1..20: mol_type = transcribed RNA
gccggggctttgggtgaggg

SEQ ID NO: 279 - RNA, 21 nt, Homo sapiens
  source 1..21: mol_type = transcribed RNA
gtaggtgacagtcaggggcgg

SEQ ID NO: 280 - RNA, 24 nt, Homo sapiens
  source 1..24: mol_type = transcribed RNA
tgtaggcatgaggcagggcccagg

SEQ ID NO: 281 - RNA, 22 nt, Homo sapiens
  source 1..22: mol_type = transcribed RNA
gcccaggactttgtgcggggtg

SEQ ID NO: 282 - RNA, 23 nt, Homo sapiens
  source 1..23: mol_type = transcribed RNA
tgggggctgggatgggccatggt

SEQ ID NO: 283 - RNA, 23 nt, Homo sapiens
  source 1..23: mol_type = transcribed RNA
aggaggtggtactaggggccagc

SEQ ID NO: 284 - RNA, 19 nt, Homo sapiens
  source 1..19: mol_type = transcribed RNA
tgggggagatgggggttga

SEQ ID NO: 285 - RNA, 21 nt, Homo sapiens
  source 1..21: mol_type = transcribed RNA
cccatgcctcctgccgcggtc

SEQ ID NO: 286 - RNA, 21 nt, Homo sapiens
  source 1..21: mol_type = transcribed RNA
tgagggacccaggacaggaga

SEQ ID NO: 287 - RNA, 22 nt, Homo sapiens
  source 1..22: mol_type = transcribed RNA
agggccgaagggtggaagctgc

SEQ ID NO: 288 - RNA, 22 nt, Homo sapiens
  source 1..22: mol_type = transcribed RNA
cagggcagggaaggtgggagag

SEQ ID NO: 289 - RNA, 22 nt, Homo sapiens
  source 1..22: mol_type = transcribed RNA
tggtggaggaagagggcagctc

SEQ ID NO: 290 - RNA, 25 nt, Homo sapiens
  source 1..25: mol_type = transcribed RNA
aggggggcactgcgcaagcaaagcc

SEQ ID NO: 291 - RNA, 23 nt, Homo sapiens
  source 1..23: mol_type = transcribed RNA
tggggggacagatggagaggaca

SEQ ID NO: 292 - RNA, 21 nt, Homo sapiens
  source 1..21: mol_type = transcribed RNA
gtgtggccggcaggcgggtgg

SEQ ID NO: 293 - RNA, 22 nt, Homo sapiens
  source 1..22: mol_type = transcribed RNA
gggacccagggagagacgtaag

SEQ ID NO: 294 - RNA, 23 nt, Homo sapiens
  source 1..23: mol_type = transcribed RNA
cctccctgcccgcctctctgcag

SEQ ID NO: 295 - RNA, 22 nt, Homo sapiens
  source 1..22: mol_type = transcribed RNA
tgcggggctagggctaacagca

SEQ ID NO: 296 - RNA, 20 nt, Homo sapiens
  source 1..20: mol_type = transcribed RNA
cggctctgggtctgtgggga

SEQ ID NO: 297 - RNA, 21 nt, Homo sapiens
  source 1..21: mol_type = transcribed RNA
aagggacagggagggtcgtgg

SEQ ID NO: 298 - RNA, 21 nt, Homo sapiens
  source 1..21: mol_type = transcribed RNA
cgtggaggacgaggaggaggc

SEQ ID NO: 299 - RNA, 18 nt, Homo sapiens
  source 1..18: mol_type = transcribed RNA
ttcccagccaacgcacca

SEQ ID NO: 300 - RNA, 22 nt, Homo sapiens
  source 1..22: mol_type = transcribed RNA
ggggaactgtagatgaaaaggc

SEQ ID NO: 301 - RNA, 22 nt, Homo sapiens
  source 1..22: mol_type = transcribed RNA
tcaaaatcaggagtcggggctt

SEQ ID NO: 302 - RNA, 20 nt, Homo sapiens
  source 1..20: mol_type = transcribed RNA
ggcggcggggaggtaggcag

SEQ ID NO: 303 - RNA, 22 nt, Homo sapiens
  source 1..22: mol_type = transcribed RNA
ctgccctggcccgagggaccga

SEQ ID NO: 304 - RNA, 22 nt, Homo sapiens
  source 1..22: mol_type = transcribed RNA
gggtggggatttgttgcattac

SEQ ID NO: 305 - RNA, 22 nt, Homo sapiens
  source 1..22: mol_type = transcribed RNA
agggacgggacgcggtgcagtg

SEQ ID NO: 306 - RNA, 21 nt, Homo sapiens
  source 1..21: mol_type = transcribed RNA
aaggcagggcccccgctcccc

SEQ ID NO: 307 - RNA, 20 nt, Homo sapiens
  source 1..20: mol_type = transcribed RNA
tcacacctgcctcgcccccc

SEQ ID NO: 308 - RNA, 17 nt, Homo sapiens
  source 1..17: mol_type = transcribed RNA
gtgggggagaggctgtc

SEQ ID NO: 309 - RNA, 22 nt, Homo sapiens
  source 1..22: mol_type = transcribed RNA
actcggcgtggcgtcggtcgtg

SEQ ID NO: 310 - RNA, 22 nt, Homo sapiens
  source 1..22: mol_type = transcribed RNA
tggggagcggcccccgggtggg

SEQ ID NO: 311 - RNA, 21 nt, Homo sapiens
  source 1..21: mol_type = transcribed RNA
atcacattgccagggatttcc

SEQ ID NO: 312 - RNA, 23 nt, Homo sapiens
  source 1..23: mol_type = transcribed RNA
tagcaccatttgaaatcagtgtt

SEQ ID NO: 313 - RNA, 22 nt, Homo sapiens
  source 1..22: mol_type = transcribed RNA
ggctggagcgagtgcagtggtg

SEQ ID NO: 314 - RNA, 23 nt, Homo sapiens
  source 1..23: mol_type = transcribed RNA
agaagaaggcggtcggtctgcgg

SEQ ID NO: 315 - RNA, 17 nt, Homo sapiens
  source 1..17: mol_type = transcribed RNA
ccccggggagcccggcg

SEQ ID NO: 316 - RNA, 22 nt, Homo sapiens
  source 1..22: mol_type = transcribed RNA
gagcaggcgaggctgggctgaa

SEQ ID NO: 317 - RNA, 23 nt, Homo sapiens
  source 1..23: mol_type = transcribed RNA
gtgagtgggagccggtggggctg

SEQ ID NO: 318 - RNA, 23 nt, Homo sapiens
  source 1..23: mol_type = transcribed RNA
ccccggtgttggggcgcgtctgc

SEQ ID NO: 319 - RNA, 19 nt, Homo sapiens
  source 1..19: mol_type = transcribed RNA
ggctggtcagatgggagtg

SEQ ID NO: 320 - RNA, 22 nt, Homo sapiens
  source 1..22: mol_type = transcribed RNA
gactatagaactttccccctca

SEQ ID NO: 321 - RNA, 23 nt, Homo sapiens
  source 1..23: mol_type = transcribed RNA
caggcagaagtggggctgacagg

SEQ ID NO: 322 - RNA, 21 nt, Homo sapiens
  source 1..21: mol_type = transcribed RNA
tcacctggctggcccgcccag

SEQ ID NO: 323 - RNA, 21 nt, Homo sapiens
  source 1..21: mol_type = transcribed RNA
tggggcggggcaggtccctgc

SEQ ID NO: 324 - RNA, 22 nt, Homo sapiens
  source 1..22: mol_type = transcribed RNA
tggggaggtgtggagtcagcat

SEQ ID NO: 325 - RNA, 19 nt, Homo sapiens
  source 1..19: mol_type = transcribed RNA
cggggccagagcagagagc

SEQ ID NO: 326 - RNA, 18 nt, Homo sapiens
  source 1..18: mol_type = transcribed RNA
ctggcagggggagaggta

SEQ ID NO: 327 - RNA, 19 nt, Homo sapiens
  source 1..19: mol_type = transcribed RNA
ttgatctcggaagctaagc

SEQ ID NO: 328 - RNA, 18 nt, Homo sapiens
  source 1..18: mol_type = transcribed RNA
atcctagtcacggcacca

SEQ ID NO: 329 - RNA, 22 nt, Homo sapiens
  source 1..22: mol_type = transcribed RNA
tattgcacttgtcccggcctgt

SEQ ID NO: 330 - RNA, 61 nt, Homo sapiens
  source 1..61: mol_type = transcribed RNA
tcggctggcgggggtagagctggctgcaggcccggcccctctcagctgctgccctctccag

SEQ ID NO: 331 - RNA, 75 nt, Homo sapiens
  source 1..75: mol_type = transcribed RNA
gtagttgttctacagaagacctggatgtgtaggagctaagacacactccaggggagctgtggaagcagtaacacg

SEQ ID NO: 332 - RNA, 83 nt, Homo sapiens
  source 1..83: mol_type = transcribed RNA
aatagagggtgcacaggcacgggagctcaggtgaggcagggagctgagctcacctgacctcccatgcctgtgcaccctctatt

SEQ ID NO: 333 - RNA, 86 nt, Homo sapiens
  source 1..86: mol_type = transcribed RNA
tttggtacttgaagagaggataccctttgtatgttcacttgattaatggcgaatatacagggggagactcttatttgcgtatcaaa

SEQ ID NO: 334 - RNA, 85 nt, Homo sapiens
  source 1..85: mol_type = transcribed RNA
ggcctgggtaggcttgcatgggggactgggaagagaccatgaacaggttagtccagggagttctcatcaagcctttactcagtag

SEQ ID NO: 335 - RNA, 80 nt, Homo sapiens
  source 1..80: mol_type = transcribed RNA
ggttggctataactatcatttccaaggttgtgcttttaggaaatgttggctgtcctgcggagagagaatggggagccagg

SEQ ID NO: 336 - RNA, 86 nt, Homo sapiens
  source 1..86: mol_type = transcribed RNA
agcactgcccccggtgagtcagggtggggctggccccctgcttcgtgcccatccgcgctctgactctctgcccacctgcaggagct

SEQ ID NO: 337 - RNA, 81 nt, Homo sapiens
  source 1..81: mol_type = transcribed RNA
tccactgctgccgccgtcgccgccacccgagccggagcgggctgggccgccaaggcaagatggtggactacagcgtgtggg

SEQ ID NO: 338 - RNA, 86 nt, Homo sapiens
  source 1..86: mol_type = transcribed RNA
tttggtacttaaagagaggataccctttgtatgttcacttgattaatggcgaatatacagggggagactctcatttgcgtatcaaa

SEQ ID NO: 339 - RNA, 78 nt, Homo sapiens
  source 1..78: mol_type = transcribed RNA
gtagctgaggggatggtagaccggtgacgtgcacttcatttacgatgtaggtcacccgtttgactatccaccagcgcc

SEQ ID NO: 340 - RNA, 87 nt, Homo sapiens
  source 1..87: mol_type = transcribed RNA
gcagggctggcagggaggctgggaggggctggctgggtctggtagtgggcatcagctggccctcatttcttaagacagcacttctgt

SEQ ID NO: 341 - RNA, 83 nt, Homo sapiens
  source 1..83: mol_type = transcribed RNA
gtgagtgggagccccagtgtgtggttggggccatggcgggtgggcagcccagcctctgagccttcctcgtctgtctgccccag

SEQ ID NO: 342 - RNA, 73 nt, Homo sapiens
  source 1..73: mol_type = transcribed RNA
tgtatccttgaatggatttttggagcaggagtggacacctgacccaaaggaaatcaatccataggctagcaat

SEQ ID NO: 343 - RNA, 66 nt, Homo sapiens
  source 1..66: mol_type = transcribed RNA
gggaggagggaggagatgggccaagttccctctggctggaacgcccttcccccccttcttcacctg

SEQ ID NO: 344 - RNA, 66 nt, Homo sapiens
  source 1..66: mol_type = transcribed RNA
cctgggaacgggttccggcagacgctgaggttgcgttgacgctcgcgccccggctcccgttccagg

SEQ ID NO: 345 - RNA, 47 nt, Homo sapiens
  source 1..47: mol_type = transcribed RNA
ctcggcgcggggcgcgggctccgggttggggcgagccaacgccgggg

SEQ ID NO: 346 - RNA, 61 nt, Homo sapiens
  source 1..61: mol_type = transcribed RNA
gccctccgcccgtgcaccccggggcaggagaccccgcgggacgcgccgaggtaggggggac

SEQ ID NO: 347 - RNA, 75 nt, Homo sapiens
  source 1..75: mol_type = transcribed RNA
ggctgtgccgggtagagagggcagtgggaggtaagagctcttcacccttcaccaccttctccacccagcatggcc

SEQ ID NO: 348 - RNA, 71 nt, Homo sapiens
  source 1..71: mol_type = transcribed RNA
tgacgggcgagcttttggcccgggttatacctgatgctcacgtataagacgagcaaaaagcttgttggtca

SEQ ID NO: 349 - RNA, 75 nt, Homo sapiens
  source 1..75: mol_type = transcribed RNA
caggggtttggggaaacggccgctgagtgaggcgtcggctgtgtttctcaccgcggtcttttcctcccactcttg

SEQ ID NO: 350 - RNA, 110 nt, Homo sapiens
  source 1..110: mol_type = transcribed RNA
tcacctggccatgtgacttgtgggcttccctttgtcatccttcgcctagggctctgagcagggcagggacagcaaaggggtgctcagttgtcacttcccacagcacggag

SEQ ID NO: 351 - RNA, 81 nt, Homo sapiens
  source 1..81: mol_type = transcribed RNA
ggacaggcacctgaggctctgttagccttggctctgggtcctgctccttagagcagaggcagagaggctcagggtctgtct

SEQ ID NO: 352 - RNA, 73 nt, Homo sapiens
  source 1..73: mol_type = transcribed RNA
accagctctgttgggacaagaggacggtcttcttttggaaggaagaccatcatcttgtccgaagagagctggt

SEQ ID NO: 353 - RNA, 61 nt, Homo sapiens
  source 1..61: mol_type = transcribed RNA
tcacccggtgagggcgggtggaggaggagggtccccaccatcagccttcactgggacggga

SEQ ID NO: 354 - RNA, 75 nt, Homo sapiens
  source 1..75: mol_type = transcribed RNA
gcagaagaaagatctggaagtgggagacacttttactatatatagtggctcccacttccagatctttctctctgt

SEQ ID NO: 355 - RNA, 77 nt, Homo sapiens
  source 1..77: mol_type = transcribed RNA
tgcagaagaaagatctggaagtgggagacactttcactatatatagtggctcccacttccagatctttctctctgta

SEQ ID NO: 356 - RNA, 77 nt, Homo sapiens
  source 1..77: mol_type = transcribed RNA
tgcagaagaaagatctggaagtgggagacactttcactatatatagtggctcccacttcctgatctttctctctgta

SEQ ID NO: 357 - RNA, 81 nt, Homo sapiens
  source 1..81: mol_type = transcribed RNA
attcaggccggtcctgcagagaggaagcccttctgcttacaggtattggaagggcttcctctctgcaggaccggcctgaat

SEQ ID NO: 358 - RNA, 81 nt, Homo sapiens
  source 1..81: mol_type = transcribed RNA
attcaggccggtcctgcagagaggaagcccttccaatacctgtaagcagaagggcttcctctctgcaggaccggcctgaat

SEQ ID NO: 359 - RNA, 85 nt, Homo sapiens
  source 1..85: mol_type = transcribed RNA
ggacctgccctgggctttctagtctcagctctcctccagctcagctggtcaggagagctgagactagaaagcccagggcaggttc

SEQ ID NO: 360 - RNA, 81 nt, Homo sapiens
  source 1..81: mol_type = transcribed RNA
acctgccctgggctttctagtctcagctctcctgaccagctgagctggaggagagctgagactagaaagcccagggcaggt

SEQ ID NO: 361 - RNA, 94 nt, Homo sapiens
  source 1..94: mol_type = transcribed RNA
cagtgcgacgggcggagcttccagacgctccgccccacgtcgcatgcgccccgggaaagcgtggggcggagcttccggaggccccgccctgctg

SEQ ID NO: 362 - RNA, 88 nt, Homo sapiens
  source 1..88: mol_type = transcribed RNA
gcgacgggcggagcttccagacgctccgccccacgtcgcatgcgccccgggaaagcgtggggcggagcttccggaggccccgccctgc

SEQ ID NO: 363 - RNA, 94 nt, Homo sapiens; identical to SEQ ID NO: 361
  source 1..94: mol_type = transcribed RNA

SEQ ID NO: 364 - RNA, 76 nt, Homo sapiens
  source 1..76: mol_type = transcribed RNA
ggggtcacctctctggccgtctaccttccacactgacaagggccgtggggacgtagctggccagacaggtgacccc

SEQ ID NO: 365 - RNA, 73 nt, Homo sapiens
  source 1..73: mol_type = transcribed RNA
aggtggcagggccagccaccaggagggctgcgtgccacccgggcagctctgctgctcactggcagtgtcacct

SEQ ID NO: 366 - RNA, 79 nt, Homo sapiens
  source 1..79: mol_type = transcribed RNA
aattaatccctctctttctagttcttcctagagtgaggaaaagctgggttgagagggcaaacaaattaactaattaatt

SEQ ID NO: 367 - RNA, 138 nt, Homo sapiens
  source 1..138: mol_type = transcribed RNA
tgttattttttgtcttctacctaagaattctgtctcttaggctttctcttcccagatttcccaaagttgggaaaagctgggttgagagggcaaaaggaaaaaaaaagaattctgtctctgacataattagatagggaa

SEQ ID NO: 368 - RNA, 75 nt, Homo sapiens
  source 1..75: mol_type = transcribed RNA
tggagtgggggggcaggaggggctcagggagaaagtgcatacagcccctggccctctctgcccttccgtcccctg

SEQ ID NO: 369 - RNA, 73 nt, Homo sapiens
  source 1..73: mol_type = transcribed RNA
aagagccgcggcgtaacggcagccatcttgtttgtttgagtgaatcggaaaggaggcgccggctgtggcggcg

SEQ ID NO: 370 - RNA, 83 nt, Homo sapiens
  source 1..83: mol_type = transcribed RNA
acggcatctttgcactcagcaggcaggctggtgcagcccgtggtgggggaccatcctgcctgctgtggggtaaggacggctgt

SEQ ID NO: 371 - RNA, 79 nt, Homo sapiens
  source 1..79: mol_type = transcribed RNA
gtgaggtgggggccagcagggagtgggctgggctgggctgggccaaggtacaaggcctcaccctgcatcccgcacccag

SEQ ID NO: 372 - RNA, 75 nt, Homo sapiens
  source 1..75: mol_type = transcribed RNA
agacagagaagccaggtcacgtctctgcagttacacagctcacgagtgcctgctggggtggaacctggtctgtct

SEQ ID NO: 373 - RNA, 69 nt, Homo sapiens
  source 1..69: mol_type = transcribed RNA
gggatactcaaaatgggggcgctttcctttttgtctgtactgggaagtgcttcgattttggggtgtccc

SEQ ID NO: 374 - RNA, 93 nt, Homo sapiens
  source 1..93: mol_type = transcribed RNA
ggcgcttttgtgcgcgcccgggtctgttggtgctcagagtgtggtcaggcggctcggactgagcaggtgggtgcggggctcggaggaggcggc

SEQ ID NO: 375 - RNA, 106 nt, Homo sapiens
  source 1..106: mol_type = transcribed RNA
agaagaatgcccaaccagccctcagttgctacagttccctgttgtttcagctcgacaacaacaggcggctgtagcaatggggggctggatgggcatctcaatgtgc

SEQ ID NO: 376 - RNA, 101 nt, Homo sapiens
  source 1..101: mol_type = transcribed RNA
gatgggccccttgtgtcctgaattgggtgggggctctgagtggggaaagtgggggcctaggggaggtcacagttgggtctaggggtcaggagggcccagga

SEQ ID NO: 377 - RNA, 62 nt, Homo sapiens
  source 1..62: mol_type = transcribed RNA
gctgggggtcccccgacagtgtggagctggggccgggtcccggggaggggggttctgggcag

SEQ ID NO: 378 - RNA, 76 nt, Homo sapiens
  source 1..76: mol_type = transcribed RNA
ccgatgcctcgggagtctacagcagggccatgtctgtgagggcccaagggtgcatgtgtctcccaggtttcggtgc

SEQ ID NO: 379 - RNA, 68 nt, Homo sapiens
  source 1..68: mol_type = transcribed RNA
ctcaggctcagtggtgcatgcttatagtcccagccactctggaggctgaaggaagatggcttgagcct

SEQ ID NO: 380 - RNA, 73 nt, Homo sapiens
  source 1..73: mol_type = transcribed RNA
ttggcaggtgccatgttgcctgctccttactgtacacgtggctggcaaggagacgggaacatggagccgccat

SEQ ID NO: 381 - RNA, 73 nt, Homo sapiens
  source 1..73: mol_type = transcribed RNA
agggagaaaagctgggctgagaggcgactggtgtctaatttgtttgtctctccaactcagactgcctggccca

SEQ ID NO: 382 - RNA, 81 nt, Homo sapiens
  source 1..81: mol_type = transcribed RNA
catcctccttacgtcccaccccccactcctgtttctggtgaaatattcaaacaggagtgggggtgggacataaggaggata

SEQ ID NO: 383 - RNA, 91 nt, Homo sapiens
  source 1..91: mol_type = transcribed RNA
gttctagagcatggtttctcatcatttgcactactgatacttggggtcagataattgtttgtggtgggggctgttgtttgcattgtaggat

SEQ ID NO: 384 - RNA, 66 nt, Homo sapiens
  source 1..66: mol_type = transcribed RNA
agcagccctcggcggcccggggggcgggcggcggtgcccgtcccggggctgcgcgaggcacaggcg

SEQ ID NO: 385 - RNA, 66 nt, Homo sapiens
  source 1..66: mol_type = transcribed RNA
acccaggaggcggaggaggtggaggttgcagtgagccaagatcgtggcactgactccagcctgggg

SEQ ID NO: 386 - RNA, 71 nt, Homo sapiens
  source 1..71: mol_type = transcribed RNA
gcagaggtgagttgacctccacagggccacccagggagtaagtagccaagtggaagttactttacctctgt

SEQ ID NO: 387 - RNA, 57 nt, Homo sapiens
  source 1..57: mol_type = transcribed RNA
agaggcaccgcctgcccagtgacatgcgtttaacggccgcggtaccctaactgtgca

SEQ ID NO: 388 - RNA, 63 nt, Homo sapiens
  source 1..63: mol_type = transcribed RNA
gcatgctgggcgaggctggcatctagcacaggcggtagatgcttgctcttgccattgcaatga

SEQ ID NO: 389 - RNA, 62 nt, Homo sapiens
  source 1..62: mol_type = transcribed RNA
ggtagggggcgggctccggcgctgggaccccactagggtggcgccttggccccgccccgccc

SEQ ID NO: 390 - RNA, 62 nt, Homo sapiens
  source 1..62: mol_type = transcribed RNA
gggggtggggctagtgatgcaggacgctggggactggagaagtcctgcctgaccctgtccca

SEQ ID NO: 391 - RNA, 73 nt, Homo sapiens
  source 1..73: mol_type = transcribed RNA
ggaggctgggctgggacggacacccggcctccactttctgtggcaggtacctcctccatgtcggcccgccttg

SEQ ID NO: 392 - RNA, 86 nt, Homo sapiens
  source 1..86: mol_type = transcribed RNA
attctaggtggggagactgacggctggaggcccataagctgtctaaaacttcggcccccagatttctggtctccccacttcagaac

SEQ ID NO: 393 - RNA, 81 nt, Homo sapiens
  source 1..81: mol_type = transcribed RNA
gcgggaggtgtaacaggactggactcccggcagccccagggcaggggcgtggggagctggtcctagctcagcgctcccgga

SEQ ID NO: 394 - RNA, 56 nt, Homo sapiens
  source 1..56: mol_type = transcribed RNA
cgaccgcacccgcccgaagctgggtcaaggagcccagcaggacgggagcgcggcgc

SEQ ID NO: 395 - RNA, 59 nt, Homo sapiens
  source 1..59: mol_type = transcribed RNA
aactgggtcccagtcttcacagttggtttctgacacgtggacctggctgggacgatgtg

SEQ ID NO: 396 - RNA, 79 nt, Homo sapiens
  source 1..79: mol_type = transcribed RNA
ccgggactttgtgggttctgaccccacttggatcacgccgacaacactggtcttgaagtcagaacccgcaaagtcctgg

SEQ ID NO: 397 - RNA, 90 nt, Homo sapiens
  source 1..90: mol_type = transcribed RNA
ctgtgggctgggccagggagcagctggtgggtgggaagtaagatctgacctggactccatcccacccaccccctgtttcctggcccacag

SEQ ID NO: 398 - RNA, 63 nt, Homo sapiens
  source 1..63: mol_type = transcribed RNA
actgggaagaggagctgagggacattgcggagagggtctcacattgtccctctcccttcccag

SEQ ID NO: 399 - RNA, 75 nt, Homo sapiens
  source 1..75: mol_type = transcribed RNA
aggctggcgtgggctgagggcaggaggcctgtggccggtcccaggcctcctgcttcctgggctcaggctcggttt

SEQ ID NO: 400 - RNA, 76 nt, Homo sapiens
  source 1..76: mol_type = transcribed RNA
ctgtggtggagctgagctccatggacgtgcagtggcatctgtcattgctgccttcctggagctcaggcccttgcag

SEQ ID NO: 401 - RNA, 79 nt, Homo sapiens
  source 1..79: mol_type = transcribed RNA
ctcgaggtgctgggggacgcgtgagcgcgagccgcttcctcacggctcggccgcggcgcgtagcccccgccacatcggg

SEQ ID NO: 402 - RNA, 82 nt, Homo sapiens
  source 1..82: mol_type = transcribed RNA
gctacggggagcggggaggaagtgggcgctgcttctgcgttatctggaaggagcagcccactcctgtcctgggctctgtggt

SEQ ID NO: 403 - RNA, 80 nt, Homo sapiens
  source 1..80: mol_type = transcribed RNA
ggttccggagccccggcgcgggcgggttctggggtgtagacgctgctggccagcccgccccagccgaggttctcggcacc

SEQ ID NO: 404 - RNA, 67 nt, Homo sapiens
  source 1..67: mol_type = transcribed RNA
tttaggagagagatgccgccttgctccttgaacaggaggagcaaggcggcatctctctgatactaaa

SEQ ID NO: 405 - RNA, 56 nt, Homo sapiens
  source 1..56: mol_type = transcribed RNA
gaccgagtggggtgagggcaggtggttcttcccgaagcagctctcgcctcttcgtc

SEQ ID NO: 406 - RNA, 51 nt, Homo sapiens
  source 1..51: mol_type = transcribed RNA
agctgtacctgaaaccaagcacctgtttgtgacttggcttcagttactagc

SEQ ID NO: 407 - RNA, 60 nt, Homo sapiens
  source 1..60: mol_type = transcribed RNA
ggcaggagggctgtgccaggttggctgggccaggcctgacctgccagcacctccctgcag

SEQ ID NO: 408 - RNA, 55 nt, Homo sapiens
  source 1..55: mol_type = transcribed RNA
aatctgccagcttccacagtggcagattttcccatagtgggaagctggcagattc

SEQ ID NO: 409 - RNA, 76 nt, Homo sapiens
  source 1..76: mol_type = transcribed RNA
cgcaggcctctggcggagcccattccatgccagatgctgagcgatggctggtgtgtgctgctccacaggcctggtg

SEQ ID NO: 410 - RNA, 70 nt, Homo sapiens
  source 1..70: mol_type = transcribed RNA
ctcgggcccgaccgcgccggcccgcacctcccggcccggagctgcgggctgcggtcagggcgatcccggg

SEQ ID NO: 411 - RNA, 63 nt, Homo sapiens
  source 1..63: mol_type = transcribed RNA
gccaaggactgatcctctcgggcagggagtcagaggggaccgcccgagaggatccgtccctgc

SEQ ID NO: 412 - RNA, 54 nt, Homo sapiens
  source 1..54: mol_type = transcribed RNA
agggaaggaggcttggtcttagcacggggtctaaggcccgggctttcctcccag

SEQ ID NO: 413 - RNA, 61 nt, Homo sapiens
  source 1..61: mol_type = transcribed RNA
cctgcggggacaggccagggcatctaggctgtgcacagtgacgcccctcctgcccccacag

SEQ ID NO: 414 - RNA, 72 nt, Homo sapiens
  source 1..72: mol_type = transcribed RNA
agattcagctttcccttcagagcctggctttggcatctatgaaagccaggctctgaagggaaagttgaatct

SEQ ID NO: 415 - RNA, 92 nt, Homo sapiens
  source 1..92: mol_type = transcribed RNA
cctgtccctcctgccctgcgcctgcccagccctcctgctctggtgactgaggaccgccaggcaggggctggtgctgggcggggggcggcggg

SEQ ID NO: 416 - RNA, 84 nt, Homo sapiens
  source 1..84: mol_type = transcribed RNA
cggtccagacgtggcgggggtggcggcggcatcccggacggcctgtgagggatgcgccgcccactgccccgcgccgcctgaccg

SEQ ID NO: 417 - RNA, 94 nt, Homo sapiens
  source 1..94: mol_type = transcribed RNA
gggctgacccctagggtcaggtgaggcccttggggcacagtggtgccatctcccctgtgctcccagggcctcgcctgtcccttgaggtcggccc

SEQ ID NO: 418 - RNA, 120 nt, Homo sapiens
  source 1..120: mol_type = transcribed RNA
gatccagggaaccctagagcagggggatggcagagcaaaattcatggcctacagctgcctcttgccaaactgcactggattttgtgtctcccattccccagagctgtctgaggtgctttg

SEQ ID NO: 419 - RNA, 83 nt, Homo sapiens
  source 1..83: mol_type = transcribed RNA
gctgctgttgggagaccctggtctgcactctatctgtattcttactgaagggagtgcagggcagggtttcccatacagagggc

SEQ ID NO: 420 - RNA, 85 nt, Homo sapiens
  source 1..85: mol_type = transcribed RNA
tctgaggtacccggggcagattggtgtagggtgcaaagcctgcccgccccctaagccttctgcccccaactccagcctgtcagga

SEQ ID NO: 421 - RNA, 119 nt, Homo sapiens
  source 1..119: mol_type = transcribed RNA
ccatgaggagctggcagtgggatggcctgggggtaggagcgtggcttctggagctagaccacatgggttcagatcccagcggtgcctctaactggccacaggaccttgggcagtcagct

SEQ ID NO: 422 - RNA, 115 nt, Homo sapiens
  source 1..115: mol_type = transcribed RNA
tctgaggagacctgggctgtcagaggccagggaaggggacgagggttggggaacaggtggttagcacttcatcctcgtctccctcccaggttagaagggcccccctctctgaagg

SEQ ID NO: 423 - RNA, 96 nt, Homo sapiens
  source 1..96: mol_type = transcribed RNA
agatgtgctctcctggcccatgaaatcaagcgtgggtgagacctggtgcagaacgggaaggcgacccatacttggtttcagaggctgtgagaataa

SEQ ID NO: 424 - RNA, 98 nt, Homo sapiens
  source 1..98: mol_type = transcribed RNA
agaatgggcaaatgaacagtaaatttggaggcctggggccctccctgctgctggagaagtgtttgcacgggtgggccttgtctttgaaaggaggtgga

SEQ ID NO: 425 - RNA, 55 nt, Homo sapiens
  source 1..55: mol_type = transcribed RNA
gggggctggggcgcggggaggtgctaggtcggcctcggctcccgcgccgcacccc

SEQ ID NO: 426 - RNA, 60 nt, Homo sapiens
  source 1..60: mol_type = transcribed RNA
cgctgggtccgcgcgccctgggccgggcgatgtccgcttgggggagcgaggggcggggcg

SEQ ID NO: 427 - RNA, 85 nt, Homo sapiens
  source 1..85: mol_type = transcribed RNA
ggggaggtagggaaaaggaagggggaggagaaggtgagaccaatgtcctgggtgccactcctgcccagtgcctcccttcctcgtt

SEQ ID NO: 428 - RNA, 109 nt, Homo sapiens
  source 1..109: mol_type = transcribed RNA
tgctattgtcttactgctacagcagggctggggattgcagtatccgctgttgctgctgctcccagtcctgcccctgctgctacctagtccagcctcaccgcatcccaga

SEQ ID NO: 429 - RNA, 54 nt, Homo sapiens
  source 1..54: mol_type = transcribed RNA
agcagcaggggagagagaggagtcctctagacaccgactctgtctcctgcagat

SEQ ID NO: 430 - RNA, 85 nt, Homo sapiens
  source 1..85: mol_type = transcribed RNA
gggacggggcctgcaggcagaagtggggctgacagggcagagggttgcgccccctcaccaccccttctgcctgcagcggtgggct

SEQ ID NO: 431 - RNA, 71 nt, Homo sapiens
  source 1..71: mol_type = transcribed RNA
ggggcctgcaggcagaagtggggctgacagggcagagggttgcgccccctcaccaccccttctgcctgcag

SEQ ID NO: 432 - RNA, 57 nt, Homo sapiens
  source 1..57: mol_type = transcribed RNA
cattggagggtgtggaagacatctgggccaactctgatctcttcatctaccccccag

SEQ ID NO: 433 - RNA, 81 nt, Homo sapiens
  source 1..81: mol_type = transcribed RNA
gggtaagtggaaagatggtgggccgcagaacatgtgctgagttcgtgccatatgtctgctgaccatcacctttagaagccc

SEQ ID NO: 434 - RNA, 100 nt, Homo sapiens
  source 1..100: mol_type = transcribed RNA
gctcggttgccgtggttgcgggccctgcccgcccgccagctcgctgacagcacgactcagggcggagggaagtaggtccgttggtcggtcgggaacgagg

SEQ ID NO: 435 - RNA, 66 nt, Homo sapiens
  source 1..66: mol_type = transcribed RNA
ggtaagtgcgcctcgggtgagcatgcacttaatgtgggtgtatgtcactcggctcggcccactacc

SEQ ID NO: 436 - RNA, 78 nt, Homo sapiens
  source 1..78: mol_type = transcribed RNA
ggcctcaggcaggcgcacccgaccacatgcatggctggtggcggcgtgcaggggtcgggtgggccaggctgtggggcg

SEQ ID NO: 437 - RNA, 92 nt, Homo sapiens
  source 1..92: mol_type = transcribed RNA
cgctgcgcttctgggcccgcggcgggcgtggggctgcccgggccggtcgaccagcgcgccgtagctcccgaggcccgagccgcgacccgcgg

SEQ ID NO: 438 - RNA, 65 nt, Homo sapiens
  source 1..65: mol_type = transcribed RNA
gagggtgggcgagggcggctgagcggctccatcccccggcctgctcatccccctcgccctctcag

SEQ ID NO: 439 - RNA, 70 nt, Homo sapiens
  source 1..70: mol_type = transcribed RNA
ttgggttggggtggtcggccctggagggggtttgtttgcttattcccctctgtgcttcacccctacccag

SEQ ID NO: 440 - RNA, 63 nt, Homo sapiens
  source 1..63: mol_type = transcribed RNA
accctagggtggggctggaggtggggctgaggctgagtcttcctccccttcctccctgcccag

SEQ ID NO: 441 - RNA, 86 nt, Homo sapiens
  source 1..86: mol_type = transcribed RNA
agagccggggccatggagcagcctgtgtagacggggacctgccctgcatgggcaccccctcactggctgcttcccttggtctccag

SEQ ID NO: 442 - RNA, 65 nt, Homo sapiens
  source 1..65: mol_type = transcribed RNA
ttctcctggggagtggctggggagcagacagacccaacctcatgctccccggcctctgcccccag

SEQ ID NO: 443 - RNA, 72 nt, Homo sapiens
  source 1..72: mol_type = transcribed RNA
atgagcgggtgggagcagatcttattgagagttccttctcctgctcctgattgtcttcccccaccctcacag

SEQ ID NO: 444 - RNA, 73 nt, Homo sapiens
  source 1..73: mol_type = transcribed RNA
aggccaggtgggtatggaggagccctcatatggcagttggcgagggcccagtgagcccctctctgctctccag

SEQ ID NO: 445 - RNA, 60 nt, Homo sapiens
  source 1..60: mol_type = transcribed RNA
ggtgcctcgggagggcatgggccaggccacataatgagccaaacccctgtctacccgcag

SEQ ID NO: 446 - RNA, 113 nt, Homo sapiens
  source 1..113: mol_type = transcribed RNA
gccgggtggggcggggcggcctcaggaggggcccagctcccctggatgtgctgcggtggggccggaggggcgtcacgtgcacccaagtgacgccccttctgattctgcctcag

SEQ ID NO: 447 - RNA, 98 nt, Homo sapiens
  source 1..98: mol_type = transcribed RNA
cgaggtaggggcgtcccgggcgcgcgggcgggtcccaggctgggcccctcggaggccgggtgctcactgccccgtcccggcgcccgtgtctcctccag

SEQ ID NO: 448 - RNA, 68 nt, Homo sapiens
  source 1..68: mol_type = transcribed RNA
gggcgcagggggactgggggtgagcaggcccagaacccagctcgtgctcactctcagtccctccctag

SEQ ID NO: 449 - RNA, 62 nt, Homo sapiens
  source 1..62: mol_type = transcribed RNA
ttaccttgtggggttggagagctggctggtccagcccctcagaagctctcccctccccgcag

SEQ ID NO: 450 - RNA, 72 nt, Homo sapiens
  source 1..72: mol_type = transcribed RNA
cagccaggagggaaggggctgagaacaggacctgtgctcactggggcctgcatgacccttccctccccacag

SEQ ID NO: 451 - RNA, 82 nt, Homo sapiens
  source 1..82: mol_type = transcribed RNA
acctgtaggtgacagtcaggggcggggtgtggtggggctggggctggccccctcctcacacctctcctggcatcgcccccag

SEQ ID NO: 452 - RNA, 65 nt, Homo sapiens
  source 1..65: mol_type = transcribed RNA
gagggctaggtggggggcttgaagccccgagatgcctcacgtcttcacccctctcacctaagcag

SEQ ID NO: 453 - RNA, 65 nt, Homo sapiens
  source 1..65: mol_type = transcribed RNA
ctcctctgggggtggggggctgggcgtggtggacagcgatgcatccctcgccttctcaccctcag

SEQ ID NO: 454 - RNA, 62 nt, Homo sapiens
  source 1..62: mol_type = transcribed RNA
tggcctagggggcggcttgtggagtgtatgggctgagccttgctctgctcccccgcccccag

SEQ ID NO: 455 - RNA, 92 nt, Homo sapiens
  source 1..92: mol_type = transcribed RNA
gtgagccagtggaatggagaggctgtgggcagggggagatgtgaaggaaagaactaggacccattcatccactgcattcctgcttggcccag

SEQ ID NO: 456 - RNA, 64 nt, Homo sapiens
  source 1..64: mol_type = transcribed RNA
tgaggatggggtgagatggggaggagcagccagtcctgtctcaccgctcttcccctgaccccag

SEQ ID NO: 457 - RNA, 61 nt, Homo sapiens
  source 1..61: mol_type = transcribed RNA
gagggttggggtggagggccaaggagctgggtggggtgccaagcctctgtccccaccccag

SEQ ID NO: 458 - RNA, 61 nt, Homo sapiens
  source 1..61: mol_type = transcribed RNA
tggcccagggaaccagttggggcttccgctctgcagaggctctaactggctttccctgcag

SEQ ID NO: 459 - RNA, 63 nt, Homo sapiens
  source 1..63: mol_type = transcribed RNA
gaggtgtaggggaggttgggccagggatgccttcactgtgtctctctggtcttgccaccccag

SEQ ID NO: 460 - RNA, 98 nt, Homo sapiens
  source 1..98: mol_type = transcribed RNA
cttggtcaataggaaagaggtgggacctcctggcttttcctctgcagcatggctcggacctagtgcaatgtttaagctcccctctctttcctgttcag

SEQ ID NO: 461 - RNA, 61 nt, Homo sapiens
  source 1..61: mol_type = transcribed RNA
gtgcggaacgctggccggggcgggaggggaagggacgcccggccggaacgccgcactcacg

SEQ ID NO: 462 - RNA, 67 nt, Homo sapiens
  source 1..67: mol_type = transcribed RNA
gtgaggaggggctggcagggacccctccaagttggggacggcagccagcccctgctcacccctcgcc

SEQ ID NO: 463 - RNA, 64 nt, Homo sapiens
  source 1..64: mol_type = transcribed RNA
gaggcactgggtaggtggggctccagggctcctgacacctggacctctcctccccaggcccaca

SEQ ID NO: 464 - RNA, 62 nt, Homo sapiens
  source 1..62: mol_type = transcribed RNA
gagggtggtggaggaagagggcagctcccatgactgcctgaccgccttctctcctcccccag

SEQ ID NO: 465 - RNA, 80 nt, Homo sapiens
  source 1..80: mol_type = transcribed RNA
tgccgtcggcctggggaggaggaagggcaagtccaaaggtatacagttggtctgttcattctctctttttggcctacaag

SEQ ID NO: 466 - RNA, 65 nt, Homo sapiens
  source 1..65: mol_type = transcribed RNA
gtctcctggggggaggagaccctgctctccctggcagcaagcctctcctgcccttccagattagc

SEQ ID NO: 467 - RNA, 61 nt, Homo sapiens
  source 1..61: mol_type = transcribed RNA
tccgctctgtggagtggggtgcctgtcccctgccactgggtgacccacccctctccaccag

SEQ ID NO: 468 - RNA, 59 nt, Homo sapiens
  source 1..59: mol_type = transcribed RNA
cggggtcggcggcgacgtgctcagcttggcacccaagttctgccgctccgacgcccggc

SEQ ID NO: 469 - RNA, 96 nt, Homo sapiens
  source 1..96: mol_type = transcribed RNA
gccccgccgcctggcctctggcccgctggggcgcgggctttcgctttcagtcgagggctagcgagcgcagcggagcctggagagaaggcgctgggc

SEQ ID NO: 470 - RNA, 69 nt, Homo sapiens
  source 1..69: mol_type = transcribed RNA
tggagggctgcgggactgtagagggcatgagctcaggagctcaggccagctcatggtgcaaggcctctg

SEQ ID NO: 471 - RNA, 76 nt, Homo sapiens
  source 1..76: mol_type = transcribed RNA
tttaaatcctgtttttcccacttactattctggtcagatatcccatgaagcagtgggtaggaggacaggaaaaagc

SEQ ID NO: 472 - RNA, 65 nt, Homo sapiens
  source 1..65: mol_type = transcribed RNA
cggccacatggcccaggctcttctccgagtgatctcggtggactggagtgggtggtaggtggcag

SEQ ID NO: 473 - RNA, 65 nt, Homo sapiens; identical to SEQ ID NO: 472
  source 1..65: mol_type = transcribed RNA

SEQ ID NO: 474 - RNA, 72 nt, Homo sapiens
  source 1..72: mol_type = transcribed RNA
gatttcagtgacctggcagcagggagcgtcgtcagtgtttgactgtttatggtatgtcagggagctggttcc

SEQ ID NO: 475 - RNA, 78 nt, Homo sapiens
  source 1..78: mol_type = transcribed RNA
ttagccctgcggccccacgcaccagggtaagagagactctcgcttcctgccctggcccgagggaccgactggctgggc

SEQ ID NO: 476 - RNA, 110 nt, Homo sapiens
  source 1..110: mol_type = transcribed RNA
ggctacagtctttcttcatgtgactcgtggacttccctttgtcatcctatgcctgagaatatatgaaggaggctgggaaggcaaagggacgttcaattgtcatcactggc

SEQ ID NO: 477 - RNA, 84 nt, Homo sapiens
  source 1..84: mol_type = transcribed RNA
ggcccctccttctcagccccagctcccgctcacccctgccacgtcaaaggaggcagaaggggagttgggagcagagaggggacc

SEQ ID NO: 478 - RNA, 91 nt, Homo sapiens
  source 1..91: mol_type = transcribed RNA
ggcgccccggctccccgcgcccccgatcggggccgccgctagtagtggcggcggcggaggcgggggcagcggcggcggcggcggaggcgcc

SEQ ID NO: 479 - RNA, 58 nt, Homo sapiens
  source 1..58: mol_type = transcribed RNA
agaagggtgtgtgtgtttttcctgagaataagagaaggaaggacagccaaattcttca

SEQ ID NO: 480 - RNA, 58 nt, Homo sapiens
  source 1..58: mol_type = transcribed RNA
cttcccagctgccctaagtcaggagtggctttcctgacacggagggtggcttgggaaa

SEQ ID NO: 481 - RNA, 70 nt, Homo sapiens
  source 1..70: mol_type = transcribed RNA
aaaagcctgtccctaagtccctcccagccttccagagttggtgccaggaaggatttagggacaggctttg

SEQ ID NO: 482 - RNA, 70 nt, Homo sapiens
  source 1..70: mol_type = transcribed RNA
aggacccagcggggctgggcgcgcggagcagcgctgggtgcagcgcctgcgccggcagctgcaagggccg

SEQ ID NO: 483 - RNA, 80 nt, Homo sapiens
  source 1..80: mol_type = transcribed RNA
acctgaggagccagccctcctcccgcacccaaacttggagcacttgacctttggctgttggagggggcaggctcgcgggt

SEQ ID NO: 484 - RNA, 76 nt, Homo sapiens
  source 1..76: mol_type = transcribed RNA
gagggagctgtagagcagggagcaggaagctgtgtgtgtccagccctgacctgtcctgttctgcccccagcccctc

SEQ ID NO: 485 - RNA, 74 nt, Homo sapiens
  source 1..74: mol_type = transcribed RNA
gctctagcctaattttagatctggtctgcttcagtttcactccaagcagacttgacctacaattagcctagagc

SEQ ID NO: 486 - RNA, 74 nt, Homo sapiens; identical to SEQ ID NO: 485
  source 1..74: mol_type = transcribed RNA

SEQ ID NO: 487 - RNA, 97 nt, Homo sapiens
  source 1..97: mol_type = transcribed RNA
atctgagttgggagggtccctctccaaatgtgtcttggggtgggggatcaagacacatttggagagggaacctcccaactcggcctctgccatcatt

SEQ ID NO: 488 - RNA, 60 nt, Homo sapiens
  source 1..60: mol_type = transcribed RNA
aggcctagggggtggcaggctggccatcagtgtgggctaaccctgtcctctccctcccag

SEQ ID NO: 489 - RNA, 68 nt, Homo sapiens
  source 1..68: mol_type = transcribed RNA
cagtgcagggagaaggtggaagtgcagagtgggctcacctctcgcccacactgtccccttctccccag

SEQ ID NO: 490 - RNA, 69 nt, Homo sapiens
  source 1..69: mol_type = transcribed RNA
gaggaggggaggtgtgcagggctggggtcactgactctgcttcccctgccctgcatggtgtccccacag

SEQ ID NO: 491 - RNA, 62 nt, Homo sapiens
  source 1..62: mol_type = transcribed RNA
ccttctgcggcagagctggggtcaccagccctcatgtacttgtgacttctcccctgccacag

SEQ ID NO: 492 - RNA, 74 nt, Homo sapiens
  source 1..74: mol_type = transcribed RNA
gtgcgtggtggctcgaggcgggggtgggggcctcgccctgcttgggccctccctgacctctccgctccgcacag

SEQ ID NO: 493 - RNA, 67 nt, Homo sapiens
  source 1..67: mol_type = transcribed RNA
cagcgtgggctgctgagaaggggcagggtcctccagctcattcctcctgcctcctccgtggcctcag

SEQ ID NO: 494 - RNA, 69 nt, Homo sapiens
  source 1..69: mol_type = transcribed RNA
ccgggcaggcaggtgtagggtggagcccactgtggctcctgactcagccctgctgccttcacctgccag

SEQ ID NO: 495 - RNA, 87 nt, Homo sapiens
  source 1..87: mol_type = transcribed RNA
gtgtggccggcaggcgggtgggcgggggcggccggtgggaaccccgccccgccccgcgcccgcactcacccgcccgtctccccacag

SEQ ID NO: 496 - RNA, 72 nt, Homo sapiens
  source 1..72: mol_type = transcribed RNA
ctgggggaggaaggacaggccatctgctattcgtccaccaacctgacttgatcctctcttccctcctcccag

SEQ ID NO: 497 - RNA, 82 nt, Homo sapiens
  source 1..82: mol_type = transcribed RNA
aaggagcactcactccaatttccctggactgggggcaggctgccacctcctggggacaggggattggggcaggatgttccag

SEQ ID NO: 498 - RNA, 74 nt, Homo sapiens
  source 1..74: mol_type = transcribed RNA
ccgcactctctccattacactaccctgcctcttctccatgagaggcagcggggtgtagtggatagagcacgggt

SEQ ID NO: 499 - RNA, 96 nt, Homo sapiens
  source 1..96: mol_type = transcribed RNA
cgggccccgggcgggcgggagggacgggacgcggtgcagtgttgttttttcccccgccaatattgcactcgtcccggcctccggcccccccggccc

SEQ ID NO: 500 - RNA, 84 nt, Homo sapiens
  source 1..84: mol_type = transcribed RNA
gctggcgtcggtgctggggagcggcccccgggtgggcctctgctctggcccctcctggggcccgcactctcgctctgggcccgc

SEQ ID NO: 501 - RNA, 63 nt, Homo sapiens
  source 1..63: mol_type = transcribed RNA
cttgcccgggagaaggaggtggcctggagagctgctgtctccagccgccgcctgtctccacag

SEQ ID NO: 502 - RNA, 90 nt, Homo sapiens
  source 1..90: mol_type = transcribed RNA
gagagaagcactggacttagggtcagaaggcctgagtctctctgctgcagatgggctctctgtccctgagccaagctttgtcctccctgg

SEQ ID NO: 503 - RNA, 109 nt, Homo sapiens
  source 1..109: mol_type = transcribed RNA
ggtcgggctcaccatgacacagtgtgagacctcgggctacaacacaggacccgggcgctgctctgacccctcgtgtcttgtgttgcagccggagggacgcaggtccgca

SEQ ID NO: 504 - RNA, 61 nt, Homo sapiens
  source 1..61: mol_type = transcribed RNA
gagggcagcgtgggtgtggcggaggcaggcgtgaccgtttgccgccctctcgctgctctag

SEQ ID NO: 505 - RNA, 67 nt, Homo sapiens
  source 1..67: mol_type = transcribed RNA
ccagacccctggggctgggcaggcggaaagaggtctgaactgcctctgcctccttggtctccggcag

SEQ ID NO: 506 - RNA, 78 nt, Homo sapiens
  source 1..78: mol_type = transcribed RNA
tactgccctcggcttctttacagtgctgccttgttgcatatggatcaagcagcattgtacagggctatgaaggcattg

SEQ ID NO: 507 - RNA, 78 nt, Homo sapiens
  source 1..78: mol_type = transcribed RNA
ttgtgctttcagcttctttacagtgctgccttgtagcattcaggtcaagcagcattgtacagggctatgaaagaacca

SEQ ID NO: 508 - RNA, 81 nt, Homo sapiens
  source 1..81: mol_type = transcribed RNA
ctctctgctttcagcttctttacagtgttgccttgtggcatggagttcaagcagcattgtacagggctatcaaagcacaga

SEQ ID NO: 509 - RNA, 119 nt, Homo sapiens
  source 1..119: mol_type = transcribed RNA
agcctgcgccggagccggggcctgagcccgggccgcgcaggccgtgaactcgtcgagctgcgcgtgcggccggtgctcaacctgccgggtcctggccccgcgctcccgcgcgccctgga

SEQ ID NO: 510 - RNA, 90 nt, Homo sapiens
  source 1..90: mol_type = transcribed RNA
gtgggtacggcccagtgggggggagagggacacgccctgggctctgcccagggtgcagccggactgactgagcccctgtgccgcccccag

SEQ ID NO: 511 - RNA, 73 nt, Homo sapiens
  source 1..73: mol_type = transcribed RNA
gtgggcgggggcaggtgtgtggtgggtggtggcctgcggtgagcagggccctcacacctgcctcgccccccag

SEQ ID NO: 512 - RNA, 69 nt, Homo sapiens
  source 1..69: mol_type = transcribed RNA
gtgggtagggtttgggggagagcgtgggctggggttcagggacaccctctcaccactgccctcccacag

SEQ ID NO: 513 - RNA, 82 nt, Homo sapiens
  source 1..82: mol_type = transcribed RNA
gtgagtgggaggccagggcacggcagggggagctgcagggctatgggaggggccccagcgtctgagccctgtcctcccgcag

SEQ ID NO: 514 - RNA, 82 nt, Homo sapiens; identical to SEQ ID NO: 513
  source 1..82: mol_type = transcribed RNA

SEQ ID NO: 515 - RNA, 102 nt, Homo sapiens
  source 1..102: mol_type = transcribed RNA
gtgggagggcccaggcgcgggcaggggtgggggtggcagagcgctgtcccgggggcggggccgaagcgcggcgaccgtaactccttctgctccgtcccccag

SEQ ID NO: 516 - RNA, 136 nt, Homo sapiens
  source 1..136: mol_type = transcribed RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 517 - RNA, 97 nt, Homo sapiens
  source 1..97: mol_type = transcribed RNA
ggtgggaggattgcttgagcctggaagctggagcctgcagtgaactatcattgtgccactgtactccagcctaggcaacaaaatgaaatcctgtcta

SEQ ID NO: 518 - RNA, 63 nt, Homo sapiens
  source 1..63: mol_type = transcribed RNA
ctgagcctggaagctggagcctgcagtgagctatgatcatgtccctgtactctagcctgggca

SEQ ID NO: 519 - RNA, 89 nt, Homo sapiens
  source 1..89: mol_type = transcribed RNA
tctccgtttatcccaccactgccaccattattgctactgttcagcaggtgctgctggtggtgatggtgatagtctggtgggggcggtgg

SEQ ID NO: 520 - RNA, 52 nt, Homo sapiens
  source 1..52: mol_type = transcribed RNA
tagccgggcgtggtggtgggggcctgtggtcccagctactttggaggctgag

SEQ ID NO: 521 - RNA, 50 nt, Homo sapiens
  source 1..50: mol_type = transcribed RNA
acccgggcgtggtggtgggggtgggtgcctgtaattccagctagttggga

SEQ ID NO: 522 - RNA, 100 nt, Homo sapiens
  source 1..100: mol_type = transcribed RNA
gaggtgggaggattgcttgagtcagggtggttgaggctgcagtaagttgtgatcataccactgcactccagcctgagtgacagagcaagaccttgtctca

SEQ ID NO: 523 - RNA, 82 nt, Homo sapiens
  source 1..82: mol_type = transcribed RNA
tgagctgttggattcggggccgtagcactgtctgagaggtttacatttctcacagtgaaccggtctctttttcagctgcttc

SEQ ID NO: 524 - RNA, 84 nt, Homo sapiens
  source 1..84: mol_type = transcribed RNA
tgtgcagtgggaaggggggccgatacactgtacgagagtgagtagcaggtctcacagtgaaccggtctctttccctactgtgtc

SEQ ID NO: 525 - RNA, 78 nt, Homo sapiens
  source 1..78: mol_type = transcribed RNA
gagcgtcacgttgacactcaaaaagtttcagattttggaacatttcggattttggatttttggatcagggatgctcaa

SEQ ID NO: 526 - RNA, 84 nt, Homo sapiens
  source 1..84: mol_type = transcribed RNA
ctccccatggccctgtctcccaacccttgtaccagtgctgggctcagaccctggtacaggcctgggggacagggacctggggac

SEQ ID NO: 527 - RNA, 84 nt, Homo sapiens
  source 1..84: mol_type = transcribed RNA
gtcagaataatgtcaaagtgcttacagtgcaggtagtgatatgtgcatctactgcagtgaaggcacttgtagcattatggtgac

SEQ ID NO: 528 - RNA, 80 nt, Homo sapiens
  source 1..80: mol_type = transcribed RNA
cgggaatgccgcggcggggacggcgattggtccgtatgtgtggtgccaccggccgccggctccgccccggcccccgcccc

SEQ ID NO: 529 - RNA, 80 nt, Homo sapiens
  source 1..80: mol_type = transcribed RNA
catccaggacaatggtgagtgccggtgcctgccctggggccgtccctgcgcaggggccgggtgctcaccgcatctgcccc

SEQ ID NO: 530 - RNA, 80 nt, Homo sapiens
  source 1..80: mol_type = transcribed RNA
cgtgtgagcccgccctgtgcccggcccacttctgcttcctcttagcgcaggaggggtcccgcactgggaggggccctcac

SEQ ID NO: 531 - RNA, 80 nt, Homo sapiens
  source 1..80: mol_type = transcribed RNA
tgagaggccgcaccttgccttgctgcccgggccgtgcacccgtgggccccagggcgacgcggcgggggcggccctagcga

SEQ ID NO: 532 - RNA, 92 nt, Homo sapiens
  source 1..92: mol_type = transcribed RNA
cggctggacagcgggcaacggaatcccaaaagcagctgttgtctccagagcattccagctgcgcttggatttcgtcccctgctctcctgcct

SEQ ID NO: 533 - RNA, 85 nt, Homo sapiens
  source 1..85: mol_type = transcribed RNA
ggctgagccgcagtagttcttcagtggcaagctttatgtcctgacccagctaaagctgccagttgaagaactgttgccctctgcc

SEQ ID NO: 534 - RNA, 97 nt, Homo sapiens
  source 1..97: mol_type = transcribed RNA
ctcaggtgctctggctgcttgggttcctggcatgctgatttgtgacttaagattaaaatcacattgccagggattaccacgcaaccacgaccttggc

SEQ ID NO: 535 - RNA, 68 nt, Homo sapiens
  source 1..68: mol_type = transcribed RNA
ctccggtgcctactgagctgatatcagttctcattttacacactggctcagttcagcaggaacaggag

SEQ ID NO: 536 - RNA, 73 nt, Homo sapiens
  source 1..73: mol_type = transcribed RNA
ctctgcctcccgtgcctactgagctgaaacacagttggtttgtgtacactggctcagttcagcaggaacaggg

SEQ ID NO: 537 - RNA, 80 nt, Homo sapiens
  source 1..80: mol_type = transcribed RNA
aggacccttccagagggccccccctcaatcctgttgtgcctaattcagagggttgggtggaggctctcctgaagggctct

SEQ ID NO: 538 - RNA, 63 nt, Homo sapiens
  source 1..63: mol_type = transcribed RNA
gagtcgaggactggtggaagggcctttcccctcagaccaaggccctggccccagcttcttctc

SEQ ID NO: 539 - RNA, 82 nt, Homo sapiens
  source 1..82: mol_type = transcribed RNA
ctgacttttttagggagtagaagggtggggagcatgaacaatgtttctcactccctacccctccactccccaaaaaagtcag

SEQ ID NO: 540 - RNA, 85 nt, Homo sapiens
  source 1..85: mol_type = transcribed RNA
ggcgcctcctgctctgctgtgccgccagggcctcccctagcgcgccttctggagaggctttgtgcggatacggggctggaggcct

SEQ ID NO: 541 - RNA, 64 nt, Homo sapiens
  source 1..64: mol_type = transcribed RNA
gggtgggggcggggcggcaggggcctcccccagtgccaggccccattctgcttctctcccagct

SEQ ID NO: 542 - RNA, 73 nt, Homo sapiens
  source 1..73: mol_type = transcribed RNA
ggcgaggggaggcgcaggctcggaaaggcgcgcgaggctccaggctccttcccgatccaccgctctcctcgct

SEQ ID NO: 543 - RNA, 82 nt, Homo sapiens
  source 1..82: mol_type = transcribed RNA
gcttcgctcccctccgccttctcttcccggttcttcccggagtcgggaaaagctgggttgagagggcgaaaaaggatgaggt

SEQ ID NO: 544 - RNA, 99 nt, Homo sapiens
  source 1..99: mol_type = transcribed RNA
gaaactgggctcaaggtgaggggtgctatctgtgattgagggacatggttaatggaattgtctcacacagaaatcgcacccgtcaccttggcctactta

SEQ ID NO: 545 - RNA, 85 nt, Homo sapiens
  source 1..85: mol_type = transcribed RNA
gtgagctgctggggacgcgggtcggggtctgcagggcggtgcggcagccgccacctgacgccgcgcctttgtctgtgtcccacag

SEQ ID NO: 546 - RNA, 180 nt, Homo sapiens
  source 1..180: mol_type = transcribed RNA
cgcgactgcggcggcggtggtggggggagccgcggggatcgccgagggccggtcggccgccccgggtgccgcgcggtgccgccggcggcggtgaggccccgcgcgtgtgtcccggctgcggtcggccgcgctcgaggggtccccgtggcgtccccttccccgccggccgcctttctcgcg

SEQ ID NO: 547 - RNA, 69 nt, Homo sapiens
  source 1..69: mol_type = transcribed RNA
ctttcggccagcgggacggcatccgaggtgggctaggctcgggcccgtggcgggtgcgggggtgggagg

SEQ ID NO: 548 - RNA, 87 nt, Homo sapiens
  source 1..87: mol_type = transcribed RNA
accgcagggaaaatgagggacttttgggggcagatgtgtttccattccactatcataatgcccctaaaaatccttattgctcttgca

SEQ ID NO: 549 - RNA, 105 nt, Homo sapiens
  source 1..105: mol_type = transcribed RNA
gcgggcggcggcggcggcagcagcagcaggtgcggggcggcggccgcgctggccgctcgactccgcagctgctcgttctgcttctccagcttgcgcaccagctcc

SEQ ID NO: 550 - RNA, 68 nt, Homo sapiens
  source 1..68: mol_type = transcribed RNA
cgtggtgaggatatggcagggaaggggagtttccctctattcccttccccccagtaatcttcatcatg

SEQ ID NO: 551 - RNA, 67 nt, Homo sapiens
  source 1..67: mol_type = transcribed RNA
gtggcactcaaactgtgggggcactttctgctctctggtgaaagtgccgccatcttttgagtgttac

SEQ ID NO: 552 - RNA, 102 nt, Homo sapiens
  source 1..102: mol_type = transcribed RNA
gcttatcgaggaaaagatcgaggtgggttggggcgggctctggggatttggtctcacagcccggatcccagcccacttaccttggttactctccttccttct

SEQ ID NO: 553 - RNA, 94 nt, Homo sapiens
  source 1..94: mol_type = transcribed RNA
ataaaggaagttaggctgaggggcagagagcgagacttttctattttccaaaagctcggtctgaggcccctcagtcttgcttcctaacccgcgc

SEQ ID NO: 554 - RNA, 86 nt, Homo sapiens
  source 1..86: mol_type = transcribed RNA
ggcttagaaacagtccctaggtaggatttggggaggagctaagaagcccctacagggcccagaggtggggactgagccttagttgg

SEQ ID NO: 555 - RNA, 70 nt, Homo sapiens
  source 1..70: mol_type = transcribed RNA
acaaatagcttcagggagtcaggggagggcagaaatagatggccttcccctgctgggaagaaagtgggtc

SEQ ID NO: 556 - RNA, 67 nt, Homo sapiens
  source 1..67: mol_type = transcribed RNA
aaatctctctccatatctttcctgcagcccccaggtgggggggaagaaaaggtggggaattagattc

SEQ ID NO: 557 - RNA, 93 nt, Homo sapiens
  source 1..93: mol_type = transcribed RNA
tacttatggcaccccactcctggtaccatagtcataagttaggagatgttagagctgtgagtaccatgacttaagtgtggtggcttaaacatg

SEQ ID NO: 558 - RNA, 73 nt, Homo sapiens
  source 1..73: mol_type = transcribed RNA
ggggaggtacctgggacaggaggaggaggcagccttgcctcagaaaccaaactgtcaaaagtgtaggttccac

SEQ ID NO: 559 - RNA, 73 nt, Homo sapiens
  source 1..73: mol_type = transcribed RNA
gaaaacaaccaggtgggcttcccggagggcggaacacccagccccagcatccagggctcacctaccacgtttg

SEQ ID NO: 560 - RNA, 67 nt, Homo sapiens
  source 1..67: mol_type = transcribed RNA
gcgccctccctctctccccggtgtgcaaatgtgtgtgtgcggtgttatgccggacaagagggaggtg

SEQ ID NO: 561 - RNA, 67 nt, Homo sapiens
  source 1..67: mol_type = transcribed RNA
ctggtccatttccctgccattcccttggcttcaatttactcccagggctggcagtgacatgggtcaa

SEQ ID NO: 562 - RNA, 86 nt, Homo sapiens
  source 1..86: mol_type = transcribed RNA
aggagtgaccaaaagacaagagtgcgagccttctattatgcccagacagggccaccagagggctccttggtctaggggtaatgcca

SEQ ID NO: 563 - RNA, 55 nt, Homo sapiens
  source 1..55: mol_type = transcribed RNA
ccggatccgagtcacggcaccaaatttcatgcgtgtccgtgtgaagagaccacca

SEQ ID NO: 564 - RNA, 63 nt, Homo sapiens
  source 1..63: mol_type = transcribed RNA
tggtggcggcggtagttatgggcttctctttctcaccagcagcccctgggccgccgcctccct

SEQ ID NO: 565 - RNA, 80 nt, Homo sapiens
  source 1..80: mol_type = transcribed RNA
tggcagacccttgctctctcactctccctaatggggctgaagacagctcaggggcagggtggggggtgttgtttttgttt

SEQ ID NO: 566 - RNA, 67 nt, Homo sapiens
  source 1..67: mol_type = transcribed RNA
tggtgggggtggggggtgttgtttttgtttttgagacagagtcttgctccgtcgcccaggccggagt

SEQ ID NO: 567 - RNA, 52 nt, Homo sapiens
  source 1..52: mol_type = transcribed RNA
tctgggctgagccgagctgggttaagccgagctgggttgggctgggctgggt

SEQ ID NO: 568 - RNA, 86 nt, Homo sapiens
  source 1..86: mol_type = transcribed RNA
agggagaagggtcggggcagggagggcagggcaggctctggggtggggggtctgtgagtcagccacggctctgcccacgtctcccc

SEQ ID NO: 569 - RNA, 72 nt, Homo sapiens
  source 1..72: mol_type = transcribed RNA
cttgggaatggcaaggaaaccgttaccattactgagtttagtaatggtaatggttctcttgctatacccaga

SEQ ID NO: 570 - RNA, 64 nt, Homo sapiens
  source 1..64: mol_type = transcribed RNA
tctgggcgaggggtgggctctcagaggggctggcagtactgctctgaggcctgcctctccccag

SEQ ID NO: 571 - RNA, 73 nt, Homo sapiens
  source 1..73: mol_type = transcribed RNA
cggcgacggcggggtgggtgaggtcgggccccaagactcggggtttgccgggcgcctcagttcaccgcggccg

SEQ ID NO: 572 - RNA, 87 nt, Homo sapiens
  source 1..87: mol_type = transcribed RNA
cccaggcgcccgctcccgacccacgccgcgccgccgggtccctcctccccggagaggctgggctcgggacgcgcggctcagctcggg

SEQ ID NO: 573 - RNA, 77 nt, Homo sapiens
  source 1..77: mol_type = transcribed RNA
catgagaaatcctgctggtcaaccatagccctggtcagactctccggggctgtgattgaccagcaggacttctcatg

SEQ ID NO: 574 - RNA, 70 nt, Homo sapiens
  source 1..70: mol_type = transcribed RNA
ggtttctccttgaggagacatggtgggggccggtcaggcagcccatgccatgtgtcctcatggagaggcc

SEQ ID NO: 575 - RNA, 74 nt, Homo sapiens
  source 1..74: mol_type = transcribed RNA
cctgcaggaggcagtgggcgagcaggcggggcagcccaatgccatgggcctgatctcaccgctgcctccttccc

SEQ ID NO: 576 - RNA, 78 nt, Homo sapiens
  source 1..78: mol_type = transcribed RNA
gggcccagaagggggcgcagtcactgacgtgaagggaccacatcccgcttcatgtcagtgactcctgccccttggtct

SEQ ID NO: 577 - RNA, 90 nt, Homo sapiens
  source 1..90: mol_type = transcribed RNA
gtgtctctctggagaccctgcagccttcccacccaccagggagctttccatgggctgtggggaaggcgtcagtgtcgggtgagggaacac

SEQ ID NO: 578 - RNA, 74 nt, Homo sapiens
  source 1..74: mol_type = transcribed RNA
gggaggaagaagggaggaggagcggaggggcccttgtcttcccagagcctctcccttcctcccctccccctccc

SEQ ID NO: 579 - RNA, 62 nt, Homo sapiens
  source 1..62: mol_type = transcribed RNA
gtgagtggggctcccgggacggcgcccgccctggccctggcccggcgacgtctcacggtccc

SEQ ID NO: 580 - RNA, 74 nt, Homo sapiens
  source 1..74: mol_type = transcribed RNA
gcagcccggtgagcgctcgctggcctggcagtgcgtcggaagaacagggcgggtggggccgcgcacatctctgc

SEQ ID NO: 581 - RNA, 68 nt, Homo sapiens
  source 1..68: mol_type = transcribed RNA
gcatcctgtactgagctgccccgaggcccttcatgctgcccagctcggggcagctcagtacaggatac

SEQ ID NO: 582 - RNA, 64 nt, Homo sapiens
  source 1..64: mol_type = transcribed RNA
tcctgtactgagctgccccgagctgggcagcatgaagggcctcggggcagctcagtacaggatg

SEQ ID NO: 583 - RNA, 100 nt, Homo sapiens
  source 1..100: mol_type = transcribed RNA
agctcagggcggctgcgcagagggctggactcagcggcggagctggctgctggcctcagttctgcctctgtccaggtccttgtgacccgcccgctctcct

SEQ ID NO: 584 - RNA, 115 nt, Homo sapiens
  source 1..115: mol_type = transcribed RNA
gagcaaaaaccagagaacaacatgggagcgttcctaacccctaaggcaactggatgggagacctgacccatccagttctctgagggggctcttgtgtgttctacaaggttgttca

SEQ ID NO: 585 - RNA, 97 nt, Homo sapiens
  source 1..97: mol_type = transcribed RNA
tgatgctttgctggctggtgcagtgcctgagggagtaagagccctgttgttgtaagatagtgtcttactccctcaggcacatctccaacaagtctct

SEQ ID NO: 586 - RNA, 97 nt, Homo sapiens
  source 1..97: mol_type = transcribed RNA
tgatgctttgctggctggtgcagtgcctgagggagtaagagccctgttgttgtcagatagtgtcttactccctcaggcacatctccagcgagtctct

SEQ ID NO: 587 - RNA, 72 nt, Homo sapiens
  source 1..72: mol_type = transcribed RNA
ctgtgcacctgggggagtgcagtgattgtggaatgcaaagtcccacaatcactgtactccccaggtgcacag

SEQ ID NO: 588 - RNA, 95 nt, Homo sapiens
  source 1..95: mol_type = transcribed RNA
gacaccacatgctcctccaggcctgcctgccctccaggtcatgttccagtgtcccacagatgcagcaccacggcccaggcggcattggtgtcacc

SEQ ID NO: 589 - RNA, 51 nt, Homo sapiens
  source 1..51: mol_type = transcribed RNA
agagatgaagcgggggggcggggtcttgctctattgcctacgctgatctca

SEQ ID NO: 590 - RNA, 64 nt, Homo sapiens
  source 1..64: mol_type = transcribed RNA
ccccgggcccggcgttccctccccttccgtgcgccagtggaggccggggtggggcggggcgggg

SEQ ID NO: 591 - RNA, 64 nt, Homo sapiens; identical to SEQ ID NO: 590
  source 1..64: mol_type = transcribed RNA

SEQ ID NO: 592 - RNA, 96 nt, Homo sapiens
  source 1..96: mol_type = transcribed RNA
gctctggggcgtgccgccgccgtcgctgccacctcccctaccgctagtggaagaagatggcggaaggcggagcggcggatctggacacccagcggt

SEQ ID NO: 593 - RNA, 89 nt, Homo sapiens
  source 1..89: mol_type = transcribed RNA
agcctgtgggaaagagaagagcagggcagggtgaaggcccggcggagacactctgcccaccccacaccctgcctatgggccacacagct

SEQ ID NO: 594 - RNA, 90 nt, Homo sapiens
  source 1..90: mol_type = transcribed RNA
tctaagaaacgcagtggtctctgaagcctgcaggggcaggccagccctgcactgaacgcctgttcttgccaggtggcagaaggttgctgc

SEQ ID NO: 595 - RNA, 96 nt, Homo sapiens
  source 1..96: mol_type = transcribed RNA
ctcgggaggggcgggaggggggtccccggtgctcggatctcgagggtgcttattgttcggtccgagcctgggtctccctcttccccccaacccccc

SEQ ID NO: 596 - RNA, 99 nt, Homo sapiens
  source 1..99: mol_type = transcribed RNA
cgcccacctcagcctcccaaaatgctgggattacaggcatgagccactgcggtcgaccatgacctggacatgtttgtgcccagtactgtcagtttgcag

SEQ ID NO: 597 - RNA, 100 nt, Homo sapiens
  source 1..100: mol_type = transcribed RNA
gtgagcgggcgcggcagggatcgcgggcgggtggcggcctagggcgcggagggcggaccgggaatggcgcgccgtgcgccgccggcgtaactgcggcgct

SEQ ID NO: 598 - RNA, 77 nt, Homo sapiens
  source 1..77: mol_type = transcribed RNA
gagttgggaggttccctctccaaatgtgtcttgatcccccaccccaagacacatttggagagggaccctcccaactc

SEQ ID NO: 599 - RNA, 96 nt, Homo sapiens
  source 1..96: mol_type = transcribed RNA
cagtgctggggtctcaggaggcagcgctctcaggacgtcaccaccatggcctgggctctgctcctcctcaccctcctcactcagggcacaggtgat

SEQ ID NO: 600 - RNA, 93 nt, Homo sapiens
  source 1..93: mol_type = transcribed RNA
ccttccggcgtcccaggcggggcgccgcgggaccgccctcgtgtctgtggcggtgggatcccgcggccgtgttttcctggtggcccggccatg

SEQ ID NO: 601 - RNA, 115 nt, Homo sapiens
  source 1..115: mol_type = transcribed RNA
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 602 - RNA, 73 nt, Homo sapiens
  source 1..73: mol_type = transcribed RNA
ctggtgtttgaggcgatgtggggatgtagagacaacttcccagtctcatttcctcatcctgccaggccaccat

SEQ ID NO: 603 - RNA, 87 nt, Homo sapiens
  source 1..87: mol_type = transcribed RNA
ccctcatctctgggcaggggcttattgtaggagtctctgaagagagctgtggactgacctgctttaacccttccccaggttcccatt

SEQ ID NO: 604 - RNA, 61 nt, Homo sapiens
  source 1..61: mol_type = transcribed RNA
gggggcgggagctggggtctgcaggttcgcactgatgcctgctcgccctgtctcccgctag

SEQ ID NO: 605 - RNA, 65 nt, Homo sapiens
  source 1..65: mol_type = transcribed RNA
gggtgctcggggcaggcggctgggagcggccctcacattgatggctcctgccacctcctccgcag

SEQ ID NO: 606 - RNA, 64 nt, Homo sapiens
  source 1..64: mol_type = transcribed RNA
gaaggcgaggggtagaagagcacaggggttctgataaacccttctgcctgcattctactcccag

SEQ ID NO: 607 - RNA, 63 nt, Homo sapiens
  source 1..63: mol_type = transcribed RNA
aatgggtgggtgctggtgggagccgtgccctggccactcattcggctctctccctcaccctag

SEQ ID NO: 608 - RNA, 69 nt, Homo sapiens
  source 1..69: mol_type = transcribed RNA
ggccctcgggcctggggttgggggagctctgtcctgtctcactcattgctcctcccctgcctggcccag

SEQ ID NO: 609 - RNA, 71 nt, Homo sapiens
  source 1..71: mol_type = transcribed RNA
atggaggggggtgtggagccagggggcccaggtctacagcttctccccgctccctgcccccatactcccag

SEQ ID NO: 610 - RNA, 73 nt, Homo sapiens
  source 1..73: mol_type = transcribed RNA
cccagggtctggtgcggagagggcccacagtggacttggtgacgctgtatgccctcaccgctcagcccctggg

SEQ ID NO: 611 - RNA, 69 nt, Homo sapiens
  source 1..69: mol_type = transcribed RNA
gggcttagggatgggaggccaggatgaagattaatccctaatccccaacactggccttgctatccccag

SEQ ID NO: 612 - RNA, 87 nt, Homo sapiens
  source 1..87: mol_type = transcribed RNA
gtgaggcggggccaggagggtgtgtggcgtgggtgctgcggggccgtcagggtgcctgcgggacgctcacctggctggcccgcccag

SEQ ID NO: 613 - RNA, 69 nt, Homo sapiens
  source 1..69: mol_type = transcribed RNA
gaacctcggggcatgggggagggaggctggacaggagagggctcacccaggccctgtcctctgccccag

SEQ ID NO: 614 - RNA, 79 nt, Homo sapiens
  source 1..79: mol_type = transcribed RNA
cagcctggggaaggcttggcagggaagacacatgagcagtgcctccacttcacgcctctcccttgtctcctttccctag

SEQ ID NO: 615 - RNA, 69 nt, Homo sapiens
  source 1..69: mol_type = transcribed RNA
tggggtaggggtgggggaattcaggggtgtcgaactcatggctgccacctttgtgtccccatcctgcag

SEQ ID NO: 616 - RNA, 67 nt, Homo sapiens
  source 1..67: mol_type = transcribed RNA
tacaggccggggctttgggtgagggacccccggagtctgtcacggtctcaccccaactctgccccag

SEQ ID NO: 617 - RNA, 64 nt, Homo sapiens
  source 1..64: mol_type = transcribed RNA
tgctctgtaggcatgaggcagggcccaggttccatgtgatgctgaagctctgacattcctgcag

SEQ ID NO: 618 - RNA, 71 nt, Homo sapiens
  source 1..71: mol_type = transcribed RNA
tgaccacccccgggcaaagacctgcagatcccctgttagagacgggcccaggactttgtgcggggtgccca

SEQ ID NO: 619 - RNA, 70 nt, Homo sapiens
  source 1..70: mol_type = transcribed RNA
gtccctgggggctgggatgggccatggtgtgctctgatccccctgtggtctcttggcccccaggaactcc

SEQ ID NO: 620 - RNA, 67 nt, Homo sapiens
  source 1..67: mol_type = transcribed RNA
cagggaggaggtggtactaggggccagcaacctgattacccctctttggccctttgtacccctccag

SEQ ID NO: 621 - RNA, 60 nt, Homo sapiens
  source 1..60: mol_type = transcribed RNA
caaggtgggggagatgggggttgaacttcatttctcatgctcatccccatctcctttcag

SEQ ID NO: 622 - RNA, 62 nt, Homo sapiens
  source 1..62: mol_type = transcribed RNA
gtgggtctcgcatcaggaggcaaggccaggacccgctgacccatgcctcctgccgcggtcag

SEQ ID NO: 623 - RNA, 72 nt, Homo sapiens
  source 1..72: mol_type = transcribed RNA
gagtctgagggacccaggacaggagaaggcctatggtgatttgcattcttcctgccctggctccatcctcag

SEQ ID NO: 624 - RNA, 64 nt, Homo sapiens
  source 1..64: mol_type = transcribed RNA
agttcagggccgaagggtggaagctgctggtgctcatctcagcctctgcccttggcctccccag

SEQ ID NO: 625 - RNA, 66 nt, Homo sapiens
  source 1..66: mol_type = transcribed RNA
cagagcagggcagggaaggtgggagaggggcccagctgaccctcctgtcacccgctccttgcccag

SEQ ID NO: 626 - RNA, 66 nt, Homo sapiens
  source 1..66: mol_type = transcribed RNA
cctggaggggggcactgcgcaagcaaagccagggaccctgagaggctttgcttcctgctcccctag

SEQ ID NO: 627 - RNA, 65 nt, Homo sapiens
  source 1..65: mol_type = transcribed RNA
gagaatggggggacagatggagaggacacaggctggcactgaggtcccctccactttcctcctag

SEQ ID NO: 628 - RNA, 76 nt, Homo sapiens
  source 1..76: mol_type = transcribed RNA
actgactttgagtctctcctcagggtgctgcaggcaaagctggggacccagggagagacgtaagtgaggggagatg

SEQ ID NO: 629 - RNA, 59 nt, Homo sapiens
  source 1..59: mol_type = transcribed RNA
ctccagggagacagtgtgtgaggcctcttgccatggcctccctgcccgcctctctgcag

SEQ ID NO: 630 - RNA, 98 nt, Homo sapiens
  source 1..98: mol_type = transcribed RNA
ttgggcaaggtgcggggctagggctaacagcagtcttactgaaggtttcctggaaaccacgcacatgctgttgccactaacctcaaccttactcggtc

SEQ ID NO: 631 - RNA, 80 nt, Homo sapiens
  source 1..80: mol_type = transcribed RNA
ggcgcgtcgcccccctcagtccaccagagcccggatacctcagaaattcggctctgggtctgtggggagcgaaatgcaac

SEQ ID NO: 632 - RNA, 99 nt, Homo sapiens
  source 1..99: mol_type = transcribed RNA
gcaagggacagggagggtcgtggcgacactcgcgccagctcccgggacggctgggctcgggctggtcgccgacctccgaccctccactagatgcctggc

SEQ ID NO: 633 - RNA, 103 nt, Homo sapiens
  source 1..103: mol_type = transcribed RNA
gtgtcggctgtggcgtgactgtccctctgtgtcccccactaggcccactgctcagtggagcgtggaggacgaggaggaggccgtccacgagcaatgccagcat

SEQ ID NO: 634 - RNA, 49 nt, Homo sapiens
  source 1..49: mol_type = transcribed RNA
ttcccagccaacgcaccaaaaatgatatgggtctgttgtctggagaaac

SEQ ID NO: 635 - RNA, 81 nt, Homo sapiens
  source 1..81: mol_type = transcribed RNA
tacaggtgcaggggaactgtagatgaaaaggcttggcacttgagggaaagcctcagttcattctcattttgctcacctgtt

SEQ ID NO: 636 - RNA, 81 nt, Homo sapiens
  source 1..81: mol_type = transcribed RNA
tagaggcagtttcaacagatgtgtagacttttgatatgagaaattggtttcaaaatcaggagtcggggctttactgctttt

SEQ ID NO: 637 - RNA, 80 nt, Homo sapiens
  source 1..80: mol_type = transcribed RNA
gcgtcaagatggcggcggggaggtaggcagagcaggacgccgctgctgccgccgccaccgccgcctccgctccagtcgcc

SEQ ID NO: 638 - RNA, 75 nt, Homo sapiens
  source 1..75: mol_type = transcribed RNA
tcatccctgggtggggatttgttgcattacttgtgttctatataaagtattgcacttgtcccggcctgtggaaga

SEQ ID NO: 639 - RNA, 94 nt, Homo sapiens
  source 1..94: mol_type = transcribed RNA
gtgaggtgtgggcccggccccaggagcggggcctgggcagccccgtgtgttgaggaaggaaggcagggcccccgctccccgggcctgaccccac

SEQ ID NO: 640 - RNA, 80 nt, Homo sapiens
  source 1..80: mol_type = transcribed RNA
cctctgtgagaaagggtgtgggggagaggctgtcttgtgtctgtaagtatgccaaacttattttccccaaggcagaggga

SEQ ID NO: 641 - RNA, 149 nt, Homo sapiens
  source 1..149: mol_type = transcribed RNA
catcaagacccagctgagtcactgtcactgcctaccaatctcgaccggacctcgaccggctcgtctgtgttgccaatcgactcggcgtggcgtcggtcgtggtagataggcggtcatgcatacgaattttcagctcttgttctggtgac

SEQ ID NO: 642 - RNA, 73 nt, Homo sapiens
  source 1..73: mol_type = transcribed RNA
ggccggctggggttcctggggatgggatttgcttcctgtcacaaatcacattgccagggatttccaaccgacc

SEQ ID NO: 643 - RNA, 81 nt, Homo sapiens
  source 1..81: mol_type = transcribed RNA
cttcaggaagctggtttcatatggtggtttagatttaaatagtgattgtctagcaccatttgaaatcagtgttcttggggg

SEQ ID NO: 644 - RNA, 81 nt, Homo sapiens
  source 1..81: mol_type = transcribed RNA
cttctggaagctggtttcacatggtggcttagatttttccatctttgtatctagcaccatttgaaatcagtgttttaggag

SEQ ID NO: 645 - RNA, 68 nt, Homo sapiens
  source 1..68: mol_type = transcribed RNA
tgcccaggctggagcgagtgcagtggtgcagtcagtcctagctcactgcagcctcgaactcctgggct

SEQ ID NO: 646 - RNA, 68 nt, Homo sapiens
  source 1..68: mol_type = transcribed RNA
gaatggaagaagaaggcggtcggtctgcgggagccaggccgcagagccatccgccttctgtccatgtc

SEQ ID NO: 647 - RNA, 51 nt, Homo sapiens
  source 1..51: mol_type = transcribed RNA
acagaccccggggagcccggcggtgaagctcctggtatcctgggtgtctga

SEQ ID NO: 648 - RNA, 60 nt, Homo sapiens
  source 1..60: mol_type = transcribed RNA
gagcaggcgaggctgggctgaacccgtgggtgaggagtgcagcccagctgaggcctctgc

SEQ ID NO: 649 - RNA, 71 nt, Homo sapiens
  source 1..71: mol_type = transcribed RNA
ggtgagtgggagccggtggggctggagtaagggcacgcccggggctgccccacctgctgaccaccctcccc

SEQ ID NO: 650 - RNA, 82 nt, Homo sapiens
  source 1..82: mol_type = transcribed RNA
gggaaagcggagggcgcgcccagctcccgggctgattgcgctaacagtggccccggtgttggggcgcgtctgccgctgcccc

SEQ ID NO: 651 - RNA, 109 nt, Homo sapiens
  source 1..109: mol_type = transcribed RNA
tcccgcattccctctgctttggtcaggtggtgccctccttccatgggtagagccagagatggtgggttctggctggtcagatgggagtggacagagacccggggtcctc

SEQ ID NO: 652 - RNA, 85 nt, Homo sapiens
  source 1..85: mol_type = transcribed RNA
agggtagagggatgagggggaaagttctatagtcctgtaattagatctcaggactatagaactttccccctcatccctctgccct

SEQ ID NO: 653 - RNA, 67 nt, Homo sapiens
  source 1..67: mol_type = transcribed RNA
cctgcaggcagaagtggggctgacagggcagagggttgcgccccctcaccatcccttctgcctgcag

SEQ ID NO: 654 - RNA, 67 nt, Homo sapiens; identical to SEQ ID NO: 653
  source 1..67: mol_type = transcribed RNA

SEQ ID NO: 655 - RNA, 67 nt, Homo sapiens; identical to SEQ ID NO: 653
  source 1..67: mol_type = transcribed RNA

SEQ ID NO: 656 - RNA, 67 nt, Homo sapiens; identical to SEQ ID NO: 653
  source 1..67: mol_type = transcribed RNA

SEQ ID NO: 657 - RNA, 66 nt, Homo sapiens
  source 1..66: mol_type = transcribed RNA
ccgagtggggcggggcaggtccctgcagggactgtgacactgaaggacctgcaccttcgcccacag

SEQ ID NO: 658 - RNA, 66 nt, Homo sapiens
  source 1..66: mol_type = transcribed RNA
gggcatggggaggtgtggagtcagcatggggctaggaggccccgcgctgacccgccttctccgcag

SEQ ID NO: 659 - RNA, 61 nt, Homo sapiens
  source 1..61: mol_type = transcribed RNA
aactgcggggccagagcagagagcccttgcacaccaccagcctctcctccctgtgccccag

SEQ ID NO: 660 - RNA, 94 nt, Homo sapiens
  source 1..94: mol_type = transcribed RNA
cacggtgtcccctggtggaacctggcagggggagaggtaaggtctttcagcctctccaaagcccatggtcaggtactcaggtgggggagccctg

SEQ ID NO: 661 - RNA, 61 nt, Homo sapiens
  source 1..61: mol_type = transcribed RNA
tctcgtttgatctcggaagctaagcagggttgggcctggttagtacttggatgggaaactt

SEQ ID NO: 662 - RNA, 53 nt, Homo sapiens
  source 1..53: mol_type = transcribed RNA
gtttgatctcggaagctaagcagggtcgggcctggttagtacttggatgggag

SEQ ID NO: 663 - RNA, 68 nt, Homo sapiens
  source 1..68: mol_type = transcribed RNA
gtgcaaagagcaggaggacaggggatttatctcccaagggaggtcccctgatcctagtcacggcacca

SEQ ID NO: 664 - RNA, 78 nt, Homo sapiens
  source 1..78: mol_type = transcribed RNA
ctttctacacaggttgggatcggttgcaatgctgtgtttctgtatggtattgcacttgtcccggcctgttgagtttgg

SEQ ID NO: 665 - RNA, 17 nt, Homo sapiens
  source 1..17: mol_type = transcribed RNA
caggcacgggagctcag

SEQ ID NO: 666 - RNA, 23 nt, Homo sapiens
  source 1..23: mol_type = transcribed RNA
aatatacagggggagactcttat

SEQ ID NO: 667 - RNA, 15 nt, Homo sapiens
  source 1..15: mol_type = transcribed RNA
atatacagggggaga

SEQ ID NO: 668 - RNA, 21 nt, Homo sapiens
  source 1..21: mol_type = transcribed RNA
gtgagtcagggtggggctggc

SEQ ID NO: 669 - RNA, 26 nt, Homo sapiens
  source 1..26: mol_type = transcribed RNA
caagatggtggactacagcgtgtggg

SEQ ID NO: 670 - RNA, 15 nt, Homo sapiens
  source 1..15: mol_type = transcribed RNA
aggcaagatggtgga

SEQ ID NO: 671 - RNA, 24 nt, Homo sapiens
  source 1..24: mol_type = transcribed RNA
atatacagggggagactctcattt

SEQ ID NO: 672 - RNA, 15 nt, Homo sapiens
  source 1..15: mol_type = transcribed RNA
atatacagggggaga

SEQ ID NO: 673 - RNA, 21 nt, Homo sapiens
  source 1..21: mol_type = transcribed RNA
taggtcacccgtttgactatc

SEQ ID NO: 674 - RNA, 21 nt, Homo sapiens
  source 1..21: mol_type = transcribed RNA
gaatggatttttggagcagga

SEQ ID NO: 675 - RNA, 15 nt, Homo sapiens
  source 1..15: mol_type = transcribed RNA
gaatggatttttgga

SEQ ID NO: 676 - RNA, 26 nt, Homo sapiens
  source 1..26: mol_type = transcribed RNA
aggagggaggagatgggccaagttcc

SEQ ID NO: 677 - RNA, 15 nt, Homo sapiens
  source 1..15: mol_type = transcribed RNA
gggaggagggaggag

SEQ ID NO: 678 - RNA, 15 nt, Homo sapiens
  source 1..15: mol_type = transcribed RNA
cccgcgggacgcgcc

SEQ ID NO: 679 - RNA, 26 nt, Homo sapiens
  source 1..26: mol_type = transcribed RNA
cgggtagagagggcagtgggaggtaa

SEQ ID NO: 680 - RNA, 15 nt, Homo sapiens
  source 1..15: mol_type = transcribed RNA
cgggtagagagggca

SEQ ID NO: 681 - RNA, 28 nt, Homo sapiens
  source 1..28: mol_type = transcribed RNA
ttggggaaacggccgctgagtgaggcgt

SEQ ID NO: 682 - RNA, 15 nt, Homo sapiens
  source 1..15: mol_type = transcribed RNA
ggggaaacggccgct

SEQ ID NO: 683 - RNA, 22 nt, Homo sapiens
  source 1..22: mol_type = transcribed RNA
ggcagggacagcaaaggggtgc

SEQ ID NO: 684 - RNA, 18 nt, Homo sapiens
  source 1..18: mol_type = transcribed RNA
gcagggacagcaaagggg

SEQ ID NO: 685 - RNA, 23 nt, Homo sapiens
  source 1..23: mol_type = transcribed RNA
agcagaggcagagaggctcaggg

SEQ ID NO: 686 - RNA, 15 nt, Homo sapiens
  source 1..15: mol_type = transcribed RNA
agcagaggcagagag

SEQ ID NO: 687 - RNA, 24 nt, Homo sapiens
  source 1..24: mol_type = transcribed RNA
gttgggacaagaggacggtcttct

SEQ ID NO: 688 - RNA, 20 nt, Homo sapiens
  source 1..20: mol_type = transcribed RNA
gttgggacaagaggacggtc

SEQ ID NO: 689 - RNA, 15 nt, Homo sapiens
  source 1..15: mol_type = transcribed RNA
gcgggtggaggagga

SEQ ID NO: 690 - RNA, 21 nt, Homo sapiens
  source 1..21: mol_type = transcribed RNA
aaagatctggaagtgggagac

SEQ ID NO: 691 - RNA, 22 nt, Homo sapiens
  source 1..22: mol_type = transcribed RNA
tcctgcagagaggaagcccttc

SEQ ID NO: 692 - RNA, 18 nt, Homo sapiens
  source 1..18: mol_type = transcribed RNA
cctgcagagaggaagccc

SEQ ID NO: 693 - RNA, 23 nt, Homo sapiens
  source 1..23: mol_type = transcribed RNA
tggggcggagcttccggaggccc

SEQ ID NO: 694 - RNA, 15 nt, Homo sapiens
  source 1..15: mol_type = transcribed RNA
gccccgggaaagcgt

SEQ ID NO: 695 - RNA, 20 nt, Homo sapiens
  source 1..20: mol_type = transcribed RNA
tggggacgtagctggccaga

SEQ ID NO: 696 - RNA, 20 nt, Homo sapiens
  source 1..20: mol_type = transcribed RNA
agctctgctgctcactggca

SEQ ID NO: 697 - RNA, 24 nt, Homo sapiens
  source 1..24: mol_type = transcribed RNA
gaaaagctgggttgagagggcaaa

SEQ ID NO: 698 - RNA, 15 nt, Homo sapiens
  source 1..15: mol_type = transcribed RNA
gaaaagctgggttga

SEQ ID NO: 699 - RNA, 22 nt, Homo sapiens
  source 1..22: mol_type = transcribed RNA
ggggggcaggaggggctcaggg

SEQ ID NO: 700 - RNA, 16 nt, Homo sapiens
  source 1..16: mol_type = transcribed RNA
gtgggggggcaggagg

SEQ ID NO: 701 - RNA, 22 nt, Homo sapiens
  source 1..22: mol_type = transcribed RNA
tgagtgaatcggaaaggaggcg

SEQ ID NO: 702 - RNA, 15 nt, Homo sapiens
  source 1..15: mol_type = transcribed RNA
cggaaaggaggcgcc

SEQ ID NO: 703 - RNA, 23 nt, Homo sapiens
  source 1..23: mol_type = transcribed RNA
gtgggctgggctgggctgggcca

SEQ ID NO: 704 - RNA, 15 nt, Homo sapiens
  source 1..15: mol_type = transcribed RNA
gggctgggctgggct

SEQ ID NO: 705 - RNA, 23 nt, Homo sapiens
  source 1..23: mol_type = transcribed RNA
tgcctgctggggtggaacctggt

SEQ ID NO: 706 - RNA, 15 nt, Homo sapiens
  source 1..15: mol_type = transcribed RNA
gcctgctggggtgga

SEQ ID NO: 707 - RNA, 29 nt, Homo sapiens
  source 1..29: mol_type = transcribed RNA
ggtcaggcggctcggactgagcaggtggg

SEQ ID NO: 708 - RNA, 15 nt, Homo sapiens
  source 1..15: mol_type = transcribed RNA
agagtgtggtcaggc

SEQ ID NO: 709 - RNA, 16 nt, Homo sapiens
  source 1..16: mol_type = transcribed RNA
ctgggggtcccccgac

SEQ ID NO: 710 - RNA, 15 nt, Homo sapiens
  source 1..15: mol_type = transcribed RNA
gtgtggagctggggc

SEQ ID NO: 711 - RNA, 15 nt, Homo sapiens
  source 1..15: mol_type = transcribed RNA
gaggctgaaggaaga

SEQ ID NO: 712 - RNA, 23 nt, Homo sapiens
  source 1..23: mol_type = transcribed RNA
caaggagacgggaacatggagcc

SEQ ID NO: 713 - RNA, 22 nt, Homo sapiens
  source 1..22: mol_type = transcribed RNA
aaaagctgggctgagaggcgac

SEQ ID NO: 714 - RNA, 15 nt, Homo sapiens
  source 1..15: mol_type = transcribed RNA
aaagctgggctgaga

SEQ ID NO: 715 - RNA, 23 nt, Homo sapiens
  source 1..23: mol_type = transcribed RNA
acaggagtgggggtgggacataa

SEQ ID NO: 716 - RNA, 20 nt, Homo sapiens
  source 1..20: mol_type = transcribed RNA
acaggagtgggggtgggaca

SEQ ID NO: 717 - RNA, 26 nt, Homo sapiens
  source 1..26: mol_type = transcribed RNA
gtcccggggctgcgcgaggcacaggc

SEQ ID NO: 718 - RNA, 15 nt, Homo sapiens
  source 1..15: mol_type = transcribed RNA
ggcccggggggcggg

SEQ ID NO: 719 - RNA, 23 nt, Homo sapiens
  source 1..23: mol_type = transcribed RNA
ccaggaggcggaggaggtggagg

SEQ ID NO: 720 - RNA, 15 nt, Homo sapiens
  source 1..15: mol_type = transcribed RNA
acccaggaggcggag

SEQ ID NO: 721 - RNA, 24 nt, Homo sapiens
  source 1..24: mol_type = transcribed RNA
agaggcaccgcctgcccagtgaca

SEQ ID NO: 722 - RNA, 15 nt, Homo sapiens
  source 1..15: mol_type = transcribed RNA
gaggcaccgcctgcc

SEQ ID NO: 723 - RNA, 19 nt, Homo sapiens
  source 1..19: mol_type = transcribed RNA
gctgggcgaggctggcatc

SEQ ID NO: 724 - RNA, 19 nt, Homo sapiens
  source 1..19: mol_type = transcribed RNA
agggggcgggctccggcgc

SEQ ID NO: 725 - RNA, 15 nt, Homo sapiens
  source 1..15: mol_type = transcribed RNA
gtagggggcgggctc

SEQ ID NO: 726 - RNA, 22 nt, Homo sapiens
  source 1..22: mol_type = transcribed RNA
ggtggggctagtgatgcaggac

SEQ ID NO: 727 - RNA, 19 nt, Homo sapiens
  source 1..19: mol_type = transcribed RNA
tggggctagtgatgcagga

SEQ ID NO: 728 - RNA, 30 nt, Homo sapiens
  source 1..30: mol_type = transcribed RNA
gctgggctgggacggacacccggcctccac

SEQ ID NO: 729 - RNA, 19 nt, Homo sapiens
  source 1..19: mol_type = transcribed RNA
gaggctgggctgggacgga

SEQ ID NO: 730 - RNA, 18 nt, Homo sapiens
  source 1..18: mol_type = transcribed RNA
tctaggtggggagactga

SEQ ID NO: 731 - RNA, 16 nt, Homo sapiens
  source 1..16: mol_type = transcribed RNA
gtggggagactgacgg

SEQ ID NO: 732 - RNA, 23 nt, Homo sapiens
  source 1..23: mol_type = transcribed RNA
aggactggactcccggcagcccc

SEQ ID NO: 733 - RNA, 18 nt, Homo sapiens
  source 1..18: mol_type = transcribed RNA
gcgtggggagctggtcct

SEQ ID NO: 734 - RNA, 21 nt, Homo sapiens
  source 1..21: mol_type = transcribed RNA
cccagcaggacgggagcgcgg

SEQ ID NO: 735 - RNA, 16 nt, Homo sapiens
  source 1..16: mol_type = transcribed RNA
aagctgggtcaaggag

SEQ ID NO: 736 - RNA, 25 nt, Homo sapiens
  source 1..25: mol_type = transcribed RNA
ctgggccagggagcagctggtgggt

SEQ ID NO: 737 - RNA, 20 nt, Homo sapiens
  source 1..20: mol_type = transcribed RNA
tgggccagggagcagctggt

SEQ ID NO: 738 - RNA, 26 nt, Homo sapiens
  source 1..26: mol_type = transcribed RNA
actgggaagaggagctgagggacatt

SEQ ID NO: 739 - RNA, 19 nt, Homo sapiens
  source 1..19: mol_type = transcribed RNA
actgggaagaggagctgag

SEQ ID NO: 740 - RNA, 21 nt, Homo sapiens
  source 1..21: mol_type = transcribed RNA
ctgggggacgcgtgagcgcga

SEQ ID NO: 741 - RNA, 21 nt, Homo sapiens
  source 1..21: mol_type = transcribed RNA
agcggggaggaagtgggcgct

SEQ ID NO: 742 - RNA, 16 nt, Homo sapiens
  source 1..16: mol_type = transcribed RNA
ggagccccggcgcggg

SEQ ID NO: 743 - RNA, 22 nt, Homo sapiens
  source 1..22: mol_type = transcribed RNA
aggagcaaggcggcatctctct

SEQ ID NO: 744 - RNA, 18 nt, Homo sapiens
  source 1..18: mol_type = transcribed RNA
gagcaaggcggcatctct

SEQ ID NO: 745 - RNA, 15 nt, Homo sapiens
  source 1..15: mol_type = transcribed RNA
gggtgagggcaggtg

SEQ ID NO: 746 - RNA, 17 nt, Homo sapiens
  source 1..17: mol_type = transcribed RNA
ggcaggagggctgtgcc

SEQ ID NO: 747 - RNA, 19 nt, Homo sapiens
  source 1..19: mol_type = transcribed RNA
atagtgggaagctggcaga

SEQ ID NO: 748 - RNA, 22 nt, Homo sapiens
  source 1..22: mol_type = transcribed RNA
tggcggagcccattccatgcca

SEQ ID NO: 749 - RNA, 21 nt, Homo sapiens
  source 1..21: mol_type = transcribed RNA
ctggcggagcccattccatgc

SEQ ID NO: 750 - RNA, 24 nt, Homo sapiens
  source 1..24: mol_type = transcribed RNA
gctgcgggctgcggtcagggcgat

SEQ ID NO: 751 - RNA, 20 nt, Homo sapiens
  source 1..20: mol_type = transcribed RNA
gctgcgggctgcggtcaggg

SEQ ID NO: 752 - RNA, 24 nt, Homo sapiens
  source 1..24: mol_type = transcribed RNA
ctgcggggacaggccagggcatct

SEQ ID NO: 753 - RNA, 20 nt, Homo sapiens
  source 1..20: mol_type = transcribed RNA
ctgcggggacaggccagggc

SEQ ID NO: 754 - RNA, 22 nt, Homo sapiens
  source 1..22: mol_type = transcribed RNA
agccaggctctgaagggaaagt

SEQ ID NO: 755 - RNA, 15 nt, Homo sapiens
  source 1..15: mol_type = transcribed RNA
gaagggaaagttgaa

SEQ ID NO: 756 - RNA, 15 nt, Homo sapiens
  source 1..15: mol_type = transcribed RNA
gcccactgccccgcg

SEQ ID NO: 757 - RNA, 24 nt, Homo sapiens
  source 1..24: mol_type = transcribed RNA
cagggggatggcagagcaaaattc

SEQ ID NO: 758 - RNA, 17 nt, Homo sapiens
  source 1..17: mol_type = transcribed RNA
agggggatggcagagca

SEQ ID NO: 759 - RNA, 22 nt, Homo sapiens
  source 1..22: mol_type = transcribed RNA
gggagtgcagggcagggtttcc

SEQ ID NO: 760 - RNA, 18 nt, Homo sapiens
  source 1..18: mol_type = transcribed RNA
agggagtgcagggcaggg

SEQ ID NO: 761 - RNA, 24 nt, Homo sapiens
  source 1..24: mol_type = transcribed RNA
cccggggcagattggtgtagggtg

SEQ ID NO: 762 - RNA, 17 nt, Homo sapiens
  source 1..17: mol_type = transcribed RNA
cggggcagattggtgta

SEQ ID NO: 763 - RNA, 17 nt, Homo sapiens
  source 1..17: mol_type = transcribed RNA
gatcccagcggtgcctc

SEQ ID NO: 764 - RNA, 15 nt, Homo sapiens
  source 1..15: mol_type = transcribed RNA
gatcccagcggtgcc

SEQ ID NO: 765 - RNA, 25 nt, Homo sapiens
  source 1..25: mol_type = transcribed RNA
gggacgagggttggggaacaggtgg

SEQ ID NO: 766 - RNA, 15 nt, Homo sapiens
  source 1..15: mol_type = transcribed RNA
tggggaacaggtggt

SEQ ID NO: 767 - RNA, 23 nt, Homo sapiens
  source 1..23: mol_type = transcribed RNA
gaaatcaagcgtgggtgagacct

SEQ ID NO: 768 - RNA, 20 nt, Homo sapiens
  source 1..20: mol_type = transcribed RNA
gaaatcaagcgtgggtgaga

SEQ ID NO: 769 - RNA, 15 nt, Homo sapiens
  source 1..15: mol_type = transcribed RNA
ggcgcggggaggtgc

SEQ ID NO: 770 - RNA, 18 nt, Homo sapiens
  source 1..18: mol_type = transcribed RNA
aaaaggaagggggaggag

SEQ ID NO: 771 - RNA, 16 nt, Homo sapiens
  source 1..16: mol_type = transcribed RNA
aaggaagggggaggag

SEQ ID NO: 772 - RNA, 23 nt, Homo sapiens
  source 1..23: mol_type = transcribed RNA
acagcagggctggggattgcagt

SEQ ID NO: 773 - RNA, 19 nt, Homo sapiens
  source 1..19: mol_type = transcribed RNA
tgctgctcccagtcctgcc

SEQ ID NO: 774 - RNA, 21 nt, Homo sapiens
  source 1..21: mol_type = transcribed RNA
cagcaggggagagagaggagt

SEQ ID NO: 775 - RNA, 20 nt, Homo sapiens
  source 1..20: mol_type = transcribed RNA
cagcaggggagagagaggag

SEQ ID NO: 776 - RNA, 25 nt, Homo sapiens
  source 1..25: mol_type = transcribed RNA
ctgcaggcagaagtggggctgacag

SEQ ID NO: 777 - RNA, 19 nt, Homo sapiens
  source 1..19: mol_type = transcribed RNA
caggcagaagtggggctga

SEQ ID NO: 778 - RNA, 21 nt, Homo sapiens
  source 1..21: mol_type = transcribed RNA
caactctgatctcttcatcta

SEQ ID NO: 779 - RNA, 23 nt, Homo sapiens
  source 1..23: mol_type = transcribed RNA
tggtgggccgcagaacatgtgct

SEQ ID NO: 780 - RNA, 21 nt, Homo sapiens
  source 1..21: mol_type = transcribed RNA
tggtgggccgcagaacatgtg

SEQ ID NO: 781 - RNA, 22 nt, Homo sapiens
  source 1..22: mol_type = transcribed RNA
gcacgactcagggcggagggaa

SEQ ID NO: 782 - RNA, 15 nt, Homo sapiens
  source 1..15: mol_type = transcribed RNA
agggcggagggaagt

SEQ ID NO: 783 - RNA, 25 nt, Homo sapiens
  source 1..25: mol_type = transcribed RNA
ttctgggcccgcggcgggcgtgggg

SEQ ID NO: 784 - RNA, 15 nt, Homo sapiens
  source 1..15: mol_type = transcribed RNA
cgcggcgggcgtggg

SEQ ID NO: 785 - RNA, 22 nt, Homo sapiens
  source 1..22: mol_type = transcribed RNA
tcccccggcctgctcatccccc

SEQ ID NO: 786 - RNA, 15 nt, Homo sapiens
  source 1..15: mol_type = transcribed RNA
acctctcctggcatc

SEQ ID NO: 787 - RNA, 24 nt, Homo sapiens
  source 1..24: mol_type = transcribed RNA
gcggccccacgcaccagggtaaga

SEQ ID NO: 788 - RNA, 19 nt, Homo sapiens
  source 1..19: mol_type = transcribed RNA
cggccccacgcaccagggt

SEQ ID NO: 789 - RNA, 24 nt, Homo sapiens
  source 1..24: mol_type = transcribed RNA
gaggctgggaaggcaaagggacgt

SEQ ID NO: 790 - RNA, 15 nt, Homo sapiens
  source 1..15: mol_type = transcribed RNA
gaaggaggctgggaa

SEQ ID NO: 791 - RNA, 16 nt, Homo sapiens
  source 1..16: mol_type = transcribed RNA
gaaggggagttgggag

SEQ ID NO: 792 - RNA, 18 nt, Homo sapiens
  source 1..18: mol_type = transcribed RNA
ggcagcggcggcggcggc

SEQ ID NO: 793 - RNA, 15 nt, Homo sapiens
  source 1..15: mol_type = transcribed RNA
gctccccgcgccccc

SEQ ID NO: 794 - RNA, 19 nt, Homo sapiens
  source 1..19: mol_type = transcribed RNA
cgtcccaccccccactcct

SEQ ID NO: 795 - RNA, 25 nt, Homo sapiens
  source 1..25: mol_type = transcribed RNA
caggaaggatttagggacaggcttt

SEQ ID NO: 796 - RNA, 19 nt, Homo sapiens
  source 1..19: mol_type = transcribed RNA
caggaaggatttagggaca

SEQ ID NO: 797 - RNA, 18 nt, Homo sapiens
  source 1..18: mol_type = transcribed RNA
cagcggggctgggcgcgc

SEQ ID NO: 798 - RNA, 15 nt, Homo sapiens
  source 1..15: mol_type = transcribed RNA
cagcggggctgggcg

SEQ ID NO: 799 - RNA, 20 nt, Homo sapiens
  source 1..20: mol_type = transcribed RNA
tggctgttggagggggcagg

SEQ ID NO: 800 - RNA, 15 nt, Homo sapiens
  source 1..15: mol_type = transcribed RNA
ggagggggcaggctc

SEQ ID NO: 801 - RNA, 21 nt, Homo sapiens
  source 1..21: mol_type = transcribed RNA
cagccctcctcccgcacccaa

SEQ ID NO: 802 - RNA, 15 nt, Homo sapiens
  source 1..15: mol_type = transcribed RNA
cagggagcaggaagc

SEQ ID NO: 803 - RNA, 22 nt, Homo sapiens
  source 1..22: mol_type = transcribed RNA
taattttagatctggtctgctt

SEQ ID NO: 804 - RNA, 19 nt, Homo sapiens
  source 1..19: mol_type = transcribed RNA
aattttagatctggtctgc

SEQ ID NO: 805 - RNA, 24 nt, Homo sapiens
  source 1..24: mol_type = transcribed RNA
agacacatttggagagggaacctc

SEQ ID NO: 806 - RNA, 16 nt, Homo sapiens
  source 1..16: mol_type = transcribed RNA
agacacatttggagag

SEQ ID NO: 807 - RNA, 21 nt, Homo sapiens
  source 1..21: mol_type = transcribed RNA
aggcagcggggtgtagtggat

SEQ ID NO: 808 - RNA, 24 nt, Homo sapiens
  source 1..24: mol_type = transcribed RNA
aatattgcactcgtcccggcctcc

SEQ ID NO: 809 - RNA, 15 nt, Homo sapiens
  source 1..15: mol_type = transcribed RNA
tattgcactcgtccc

SEQ ID NO: 810 - RNA, 23 nt, Homo sapiens
  source 1..23: mol_type = transcribed RNA
ctcctggggcccgcactctcgct

SEQ ID NO: 811 - RNA, 18 nt, Homo sapiens
  source 1..18: mol_type = transcribed RNA
ctcctggggcccgcactc

SEQ ID NO: 812 - RNA, 23 nt, Homo sapiens
  source 1..23: mol_type = transcribed RNA
ggctacaacacaggacccgggcg

SEQ ID NO: 813 - RNA, 21 nt, Homo sapiens
  source 1..21: mol_type = transcribed RNA
ggctacaacacaggacccggg

SEQ ID NO: 814 - RNA, 21 nt, Homo sapiens
  source 1..21: mol_type = transcribed RNA
gagggcagcgtgggtgtggcg

SEQ ID NO: 815 - RNA, 29 nt, Homo sapiens
  source 1..29: mol_type = transcribed RNA
agcagcattgtacagggctatgaaggcat

SEQ ID NO: 816 - RNA, 15 nt, Homo sapiens
  source 1..15: mol_type = transcribed RNA
agcagcattgtacag

SEQ ID NO: 817 - RNA, 28 nt, Homo sapiens
  source 1..28: mol_type = transcribed RNA
agcagcattgtacagggctatcaaagca

SEQ ID NO: 818 - RNA, 15 nt, Homo sapiens
  source 1..15: mol_type = transcribed RNA
agcagcattgtacag

SEQ ID NO: 819 - RNA, 22 nt, Homo sapiens
  source 1..22: mol_type = transcribed RNA
gtgggcgggggcaggtgtgtgg

SEQ ID NO: 820 - RNA, 15 nt, Homo sapiens
  source 1..15: mol_type = transcribed RNA
cgggggcaggtgtgt

SEQ ID NO: 821 - RNA, 19 nt, Homo sapiens
  source 1..19: mol_type = transcribed RNA
agtgggaggccagggcacg

SEQ ID NO: 822 - RNA, 15 nt, Homo sapiens
  source 1..15: mol_type = transcribed RNA
agggggagctgcagg

SEQ ID NO: 823 - RNA, 15 nt, Homo sapiens
  source 1..15: mol_type = transcribed RNA
tggcagagcgctgtc

SEQ ID NO: 824 - RNA, 22 nt, Homo sapiens
  source 1..22: mol_type = transcribed RNA
ccgggaacgtcgagactggagc

SEQ ID NO: 825 - RNA, 15 nt, Homo sapiens
  source 1..15: mol_type = transcribed RNA
cgggaacgtcgagac

SEQ ID NO: 826 - RNA, 24 nt, Homo sapiens
  source 1..24: mol_type = transcribed RNA
acgcccttcccccccttcttcacc

SEQ ID NO: 827 - RNA, 17 nt, Homo sapiens
  source 1..17: mol_type = transcribed RNA
acgcccttccccccctt

SEQ ID NO: 828 - RNA, 27 nt, Homo sapiens
  source 1..27: mol_type = transcribed RNA
agcctggaagctggagcctgcagtgaa

SEQ ID NO: 829 - RNA, 15 nt, Homo sapiens
  source 1..15: mol_type = transcribed RNA
ggtgggaggattgct

SEQ ID NO: 830 - RNA, 20 nt, Homo sapiens
  source 1..20: mol_type = transcribed RNA
atcccaccactgccaccatt

SEQ ID NO: 831 - RNA, 15 nt, Homo sapiens
  source 1..15: mol_type = transcribed RNA
atcccaccactgcca

SEQ ID NO: 832 - RNA, 21 nt, Homo sapiens
  source 1..21: mol_type = transcribed RNA
gccgggcgtggtggtgggggc

SEQ ID NO: 833 - RNA, 15 nt, Homo sapiens
  source 1..15: mol_type = transcribed RNA
tagccgggcgtggtg

SEQ ID NO: 834 - RNA, 24 nt, Homo sapiens
  source 1..24: mol_type = transcribed RNA
cgggcgtggtggtgggggtgggtg

SEQ ID NO: 835 - RNA, 15 nt, Homo sapiens
  source 1..15: mol_type = transcribed RNA
cgggcgtggtggtgg

SEQ ID NO: 836 - RNA, 21 nt, Homo sapiens
  source 1..21: mol_type = transcribed RNA
cagcctgagtgacagagcaag

SEQ ID NO: 837 - RNA, 15 nt, Homo sapiens
  source 1..15: mol_type = transcribed RNA
actgcactccagcct

SEQ ID NO: 838 - RNA, 20 nt, Homo sapiens
  source 1..20: mol_type = transcribed RNA
cggggccgtagcactgtctg

SEQ ID NO: 839 - RNA, 20 nt, Homo sapiens
  source 1..20: mol_type = transcribed RNA
gggggccgatacactgtacg

SEQ ID NO: 840 - RNA, 20 nt, Homo sapiens
  source 1..20: mol_type = transcribed RNA
ggatttttggatcagggatg

SEQ ID NO: 841 - RNA, 15 nt, Homo sapiens
  source 1..15: mol_type = transcribed RNA
atttttggatcaggg

SEQ ID NO: 842 - RNA, 24 nt, Homo sapiens
  source 1..24: mol_type = transcribed RNA
ctggtacaggcctgggggacaggg

SEQ ID NO: 843 - RNA, 18 nt, Homo sapiens
  source 1..18: mol_type = transcribed RNA
ctggtacaggcctggggg

SEQ ID NO: 844 - RNA, 25 nt, Homo sapiens
  source 1..25: mol_type = transcribed RNA
actgcagtgaaggcacttgtagcat

SEQ ID NO: 845 - RNA, 15 nt, Homo sapiens
  source 1..15: mol_type = transcribed RNA
actgcagtgaaggca

SEQ ID NO: 846 - RNA, 22 nt, Homo sapiens
  source 1..22: mol_type = transcribed RNA
cgcggcggggacggcgattggt

SEQ ID NO: 847 - RNA, 17 nt, Homo sapiens
  source 1..17: mol_type = transcribed RNA
cggcggggacggcgatt

SEQ ID NO: 848 - RNA, 23 nt, Homo sapiens
  source 1..23: mol_type = transcribed RNA
tgcgcaggggccgggtgctcacc

SEQ ID NO: 849 - RNA, 19 nt, Homo sapiens
  source 1..19: mol_type = transcribed RNA
cgcaggggccgggtgctca

SEQ ID NO: 850 - RNA, 23 nt, Homo sapiens
  source 1..23: mol_type = transcribed RNA
aggaggggtcccgcactgggagg

SEQ ID NO: 851 - RNA, 15 nt, Homo sapiens
  source 1..15: mol_type = transcribed RNA
tgggaggggccctca

SEQ ID NO: 852 - RNA, 15 nt, Homo sapiens
  source 1..15: mol_type = transcribed RNA
cgcggcgggggcggc

SEQ ID NO: 853 - RNA, 29 nt, Homo sapiens
  source 1..29: mol_type = transcribed RNA
caacggaatcccaaaagcagctgttgtct

SEQ ID NO: 854 - RNA, 15 nt, Homo sapiens
  source 1..15: mol_type = transcribed RNA
caacggaatcccaaa

SEQ ID NO: 855 - RNA, 25 nt, Homo sapiens
  source 1..25: mol_type = transcribed RNA
aagctgccagttgaagaactgttgc

SEQ ID NO: 856 - RNA, 15 nt, Homo sapiens
  source 1..15: mol_type = transcribed RNA
aagctgccagttgaa

SEQ ID NO: 857 - RNA, 26 nt, Homo sapiens
  source 1..26: mol_type = transcribed RNA
aaaatcacattgccagggattaccac

SEQ ID NO: 858 - RNA, 15 nt, Homo sapiens
  source 1..15: mol_type = transcribed RNA
aatcacattgccagg

SEQ ID NO: 859 - RNA, 24 nt, Homo sapiens
  source 1..24: mol_type = transcribed RNA
actggctcagttcagcaggaacag

SEQ ID NO: 860 - RNA, 15 nt, Homo sapiens
  source 1..15: mol_type = transcribed RNA
tggctcagttcagca

SEQ ID NO: 861 - RNA, 15 nt, Homo sapiens
  source 1..15: mol_type = transcribed RNA
gagggttgggtggag

SEQ ID NO: 862 - RNA, 23 nt, Homo sapiens
  source 1..23: mol_type = transcribed RNA
gagggccccccctcaatcctgtt

SEQ ID NO: 863 - RNA, 16 nt, Homo sapiens
  source 1..16: mol_type = transcribed RNA
agggccccccctcaat

SEQ ID NO: 864 - RNA, 24 nt, Homo sapiens
  source 1..24: mol_type = transcribed RNA
tcgaggactggtggaagggccttt

SEQ ID NO: 865 - RNA, 16 nt, Homo sapiens
  source 1..16: mol_type = transcribed RNA
tcgaggactggtggaa

SEQ ID NO: 866 - RNA, 23 nt, Homo sapiens
  source 1..23: mol_type = transcribed RNA
agggagtagaagggtggggagca

SEQ ID NO: 867 - RNA, 16 nt, Homo sapiens
  source 1..16: mol_type = transcribed RNA
tagggagtagaagggt

SEQ ID NO: 868 - RNA, 26 nt, Homo sapiens
  source 1..26: mol_type = transcribed RNA
ccttctggagaggctttgtgcggata

SEQ ID NO: 869 - RNA, 15 nt, Homo sapiens
  source 1..15: mol_type = transcribed RNA
ccttctggagaggct

SEQ ID NO: 870 - RNA, 17 nt, Homo sapiens
  source 1..17: mol_type = transcribed RNA
gcggggcggcaggggcc

SEQ ID NO: 871 - RNA, 15 nt, Homo sapiens
  source 1..15: mol_type = transcribed RNA
gggggcggggcggca

SEQ ID NO: 872 - RNA, 15 nt, Homo sapiens
  source 1..15: mol_type = transcribed RNA
gcaggctcggaaagg

SEQ ID NO: 873 - RNA, 27 nt, Homo sapiens
  source 1..27: mol_type = transcribed RNA
gaaaagctgggttgagagggcgaaaaa

SEQ ID NO: 874 - RNA, 15 nt, Homo sapiens
  source 1..15: mol_type = transcribed RNA
cttctcttcccggtt

SEQ ID NO: 875 - RNA, 28 nt, Homo sapiens
  source 1..28: mol_type = transcribed RNA
aggggtgctatctgtgattgagggacat

SEQ ID NO: 876 - RNA, 15 nt, Homo sapiens
  source 1..15: mol_type = transcribed RNA
gctatctgtgattga

SEQ ID NO: 877 - RNA, 29 nt, Homo sapiens
  source 1..29: mol_type = transcribed RNA
gggagccgcggggatcgccgagggccggt

SEQ ID NO: 878 - RNA, 15 nt, Homo sapiens
  source 1..15: mol_type = transcribed RNA
ggcggcggtggtggg

SEQ ID NO: 879 - RNA, 19 nt, Homo sapiens
  source 1..19: mol_type = transcribed RNA
tggcgggtgcgggggtggg

SEQ ID NO: 880 - RNA, 15 nt, Homo sapiens
  source 1..15: mol_type = transcribed RNA
tggcgggtgcggggg

SEQ ID NO: 881 - RNA, 27 nt, Homo sapiens
  source 1..27: mol_type = transcribed RNA
tgagggacttttgggggcagatgtgtt

SEQ ID NO: 882 - RNA, 17 nt, Homo sapiens
  source 1..17: mol_type = transcribed RNA
ggacttttgggggcaga

SEQ ID NO: 883 - RNA, 18 nt, Homo sapiens
  source 1..18: mol_type = transcribed RNA
gcggcggcggcggcagca

SEQ ID NO: 884 - RNA, 15 nt, Homo sapiens
  source 1..15: mol_type = transcribed RNA
gcgggcggcggcggc

SEQ ID NO: 885 - RNA, 19 nt, Homo sapiens
  source 1..19: mol_type = transcribed RNA
tgaggatatggcagggaag

SEQ ID NO: 886 - RNA, 22 nt, Homo sapiens
  source 1..22: mol_type = transcribed RNA
actcaaactgtgggggcacttt

SEQ ID NO: 887 - RNA, 19 nt, Homo sapiens
  source 1..19: mol_type = transcribed RNA
actcaaactgtgggggcac

SEQ ID NO: 888 - RNA, 19 nt, Homo sapiens
  source 1..19: mol_type = transcribed RNA
gtgggttggggcgggctct

SEQ ID NO: 889 - RNA, 30 nt, Homo sapiens
  source 1..30: mol_type = transcribed RNA
tgaggggcagagagcgagacttttctattt

SEQ ID NO: 890 - RNA, 15 nt, Homo sapiens
  source 1..15: mol_type = transcribed RNA
tgaggggcagagagc

SEQ ID NO: 891 - RNA, 16 nt, Homo sapiens
  source 1..16: mol_type = transcribed RNA
tgggggggaagaaaag

SEQ ID NO: 892 - RNA, 22 nt, Homo sapiens
  source 1..22: mol_type = transcribed RNA
accccactcctggtaccatagt

SEQ ID NO: 893 - RNA, 15 nt, Homo sapiens
  source 1..15: mol_type = transcribed RNA
accccactcctggta

SEQ ID NO: 894 - RNA, 15 nt, Homo sapiens
  source 1..15: mol_type = transcribed RNA
aggaggaggaggcag

SEQ ID NO: 895 - RNA, 15 nt, Homo sapiens
  source 1..15: mol_type = transcribed RNA
ggtgggcttcccgga

SEQ ID NO: 896 - RNA, 20 nt, Homo sapiens
  source 1..20: mol_type = transcribed RNA
ctccccggtgtgcaaatgtg

SEQ ID NO: 897 - RNA, 15 nt, Homo sapiens
  source 1..15: mol_type = transcribed RNA
gtgtgcggtgttatg

SEQ ID NO: 898 - RNA, 23 nt, Homo sapiens
  source 1..23: mol_type = transcribed RNA
ccagggctggcagtgacatgggt

SEQ ID NO: 899 - RNA, 19 nt, Homo sapiens
  source 1..19: mol_type = transcribed RNA
cagggctggcagtgacatg

SEQ ID NO: 900 - RNA, 15 nt, Homo sapiens
  source 1..15: mol_type = transcribed RNA
cttggtctaggggta

SEQ ID NO: 901 - RNA, 21 nt, Homo sapiens
  source 1..21: mol_type = transcribed RNA
cggatccgagtcacggcacca

SEQ ID NO: 902 - RNA, 15 nt, Homo sapiens
  source 1..15: mol_type = transcribed RNA
ggatccgagtcacgg

SEQ ID NO: 903 - RNA, 25 nt, Homo sapiens
  source 1..25: mol_type = transcribed RNA
tggcggcggtagttatgggcttctc

SEQ ID NO: 904 - RNA, 29 nt, Homo sapiens
  source 1..29: mol_type = transcribed RNA
gctgggttaagccgagctgggttgggctg

SEQ ID NO: 905 - RNA, 19 nt, Homo sapiens
  source 1..19: mol_type = transcribed RNA
ctgggttgggctgggctgg

SEQ ID NO: 906 - RNA, 23 nt, Homo sapiens
  source 1..23: mol_type = transcribed RNA
agggtcggggcagggagggcagg

SEQ ID NO: 907 - RNA, 15 nt, Homo sapiens
  source 1..15: mol_type = transcribed RNA
gggagaagggtcggg

SEQ ID NO: 908 - RNA, 27 nt, Homo sapiens
  source 1..27: mol_type = transcribed RNA
aaaccgttaccattactgagtttagta

SEQ ID NO: 909 - RNA, 15 nt, Homo sapiens
  source 1..15: mol_type = transcribed RNA
gaaaccgttaccatt

SEQ ID NO: 910 - RNA, 15 nt, Homo sapiens
  source 1..15: mol_type = transcribed RNA
tctgggcgaggggtg

SEQ ID NO: 911 - RNA, 23 nt, Homo sapiens
  source 1..23: mol_type = transcribed RNA
ggtgggtgaggtcgggccccaag

SEQ ID NO: 912 - RNA, 22 nt, Homo sapiens
  source 1..22: mol_type = transcribed RNA
ctgggctcgggacgcgcggctc

SEQ ID NO: 913 - RNA, 19 nt, Homo sapiens
  source 1..19: mol_type = transcribed RNA
ctgggctcgggacgcgcgg

SEQ ID NO: 914 - RNA, 21 nt, Homo sapiens
  source 1..21: mol_type = transcribed RNA
ttgaggagacatggtgggggc

SEQ ID NO: 915 - RNA, 15 nt, Homo sapiens
  source 1..15: mol_type = transcribed RNA
ttgaggagacatggt

SEQ ID NO: 916 - RNA, 21 nt, Homo sapiens
  source 1..21: mol_type = transcribed RNA
aggaggcagtgggcgagcagg

SEQ ID NO: 917 - RNA, 23 nt, Homo sapiens
  source 1..23: mol_type = transcribed RNA
tggggaaggcgtcagtgtcgggt

SEQ ID NO: 918 - RNA, 16 nt, Homo sapiens
  source 1..16: mol_type = transcribed RNA
tggggaaggcgtcagt

SEQ ID NO: 919 - RNA, 23 nt, Homo sapiens
  source 1..23: mol_type = transcribed RNA
aagggaggaggagcggaggggcc

SEQ ID NO: 920 - RNA, 15 nt, Homo sapiens
  source 1..15: mol_type = transcribed RNA
gggaggaggagcgga

SEQ ID NO: 921 - RNA, 20 nt, Homo sapiens
  source 1..20: mol_type = transcribed RNA
tgagtggggctcccgggacg

SEQ ID NO: 922 - RNA, 15 nt, Homo sapiens
  source 1..15: mol_type = transcribed RNA
gggcggggggcggcg

SEQ ID NO: 923 - RNA, 17 nt, Homo sapiens
  source 1..17: mol_type = transcribed RNA
ggtgagcgctcgctggc

SEQ ID NO: 924 - RNA, 15 nt, Homo sapiens
  source 1..15: mol_type = transcribed RNA
cggtgagcgctcgct

SEQ ID NO: 925 - RNA, 23 nt, Homo sapiens
  source 1..23: mol_type = transcribed RNA
cggggcagctcagtacaggatac

SEQ ID NO: 926 - RNA, 15 nt, Homo sapiens
  source 1..15: mol_type = transcribed RNA
agctcagtacaggat

SEQ ID NO: 927 - RNA, 26 nt, Homo sapiens
  source 1..26: mol_type = transcribed RNA
agggctggactcagcggcggagctgg

SEQ ID NO: 928 - RNA, 17 nt, Homo sapiens
  source 1..17: mol_type = transcribed RNA
gcggcggagctggctgc

SEQ ID NO: 929 - RNA, 15 nt, Homo sapiens
  source 1..15: mol_type = transcribed RNA
gagggagtaagagcc

SEQ ID NO: 930 - RNA, 24 nt, Homo sapiens
  source 1..24: mol_type = transcribed RNA
tgggggagtgcagtgattgtggaa

SEQ ID NO: 931 - RNA, 19 nt, Homo sapiens
  source 1..19: mol_type = transcribed RNA
tgggggagtgcagtgattg

SEQ ID NO: 932 - RNA, 15 nt, Homo sapiens
  source 1..15: mol_type = transcribed RNA
tgaagcgggggggcg

SEQ ID NO: 933 - RNA, 16 nt, Homo sapiens
  source 1..16: mol_type = transcribed RNA
cgggcccggcgttccc

SEQ ID NO: 934 - RNA, 15 nt, Homo sapiens
  source 1..15: mol_type = transcribed RNA
ccgggcccggcgttc

SEQ ID NO: 935 - RNA, 23 nt, Homo sapiens
  source 1..23: mol_type = transcribed RNA
ctagtggaagaagatggcggaag

SEQ ID NO: 936 - RNA, 15 nt, Homo sapiens
  source 1..15: mol_type = transcribed RNA
tagtggaagaagatg

SEQ ID NO: 937 - RNA, 16 nt, Homo sapiens
  source 1..16: mol_type = transcribed RNA
gtgaaggcccggcgga

SEQ ID NO: 938 - RNA, 15 nt, Homo sapiens
  source 1..15: mol_type = transcribed RNA
gtgaaggcccggcgg

SEQ ID NO: 939 - RNA, 17 nt, Homo sapiens
  source 1..17: mol_type = transcribed RNA
tgcaggggcaggccagc

SEQ ID NO: 940 - RNA, 23 nt, Homo sapiens
  source 1..23: mol_type = transcribed RNA
gggggtccccggtgctcggatct

SEQ ID NO: 941 - RNA, 16 nt, Homo sapiens
  source 1..16: mol_type = transcribed RNA
tcgggaggggcgggag

SEQ ID NO: 942 - RNA, 24 nt, Homo sapiens
  source 1..24: mol_type = transcribed RNA
cccaaaatgctgggattacaggca

SEQ ID NO: 943 - RNA, 15 nt, Homo sapiens
  source 1..15: mol_type = transcribed RNA
gcccacctcagcctc

SEQ ID NO: 944 - RNA, 16 nt, Homo sapiens
  source 1..16: mol_type = transcribed RNA
ggcgcggagggcggac

SEQ ID NO: 945 - RNA, 15 nt, Homo sapiens
  source 1..15: mol_type = transcribed RNA
ggcgcggagggcgga

SEQ ID NO: 946 - RNA, 20 nt, Homo sapiens
  source 1..20: mol_type = transcribed RNA
aagacacatttggagaggga

SEQ ID NO: 947 - RNA, 16 nt, Homo sapiens
  source 1..16: mol_type = transcribed RNA
agacacatttggagag

SEQ ID NO: 948 - RNA, 27 nt, Homo sapiens
  source 1..27: mol_type = transcribed RNA
gctggggtctcaggaggcagcgctctc

SEQ ID NO: 949 - RNA, 17 nt, Homo sapiens
  source 1..17: mol_type = transcribed RNA
gctggggtctcaggagg

SEQ ID NO: 950 - RNA, 26 nt, Homo sapiens
  source 1..26: mol_type = transcribed RNA
cggtgggatcccgcggccgtgttttc

SEQ ID NO: 951 - RNA, 15 nt, Homo sapiens
  source 1..15: mol_type = transcribed RNA
ggggcgccgcgggac

SEQ ID NO: 952 - RNA, 23 nt, Homo sapiens
  source 1..23: mol_type = transcribed RNA
ggcccggccgtgcctgaggtttc

SEQ ID NO: 953 - RNA, 15 nt, Homo sapiens
  source 1..15: mol_type = transcribed RNA
ggcggtgggatcccg

SEQ ID NO: 954 - RNA, 21 nt, Homo sapiens
  source 1..21: mol_type = transcribed RNA
gaggcgatgtggggatgtaga

SEQ ID NO: 955 - RNA, 20 nt, Homo sapiens
  source 1..20: mol_type = transcribed RNA
cccagtctcatttcctcatc

SEQ ID NO: 956 - RNA, 25 nt, Homo sapiens
  source 1..25: mol_type = transcribed RNA
tgggcaggggcttattgtaggagtc

SEQ ID NO: 957 - RNA, 19 nt, Homo sapiens
  source 1..19: mol_type = transcribed RNA
tgggcaggggcttattgta

SEQ ID NO: 958 - RNA, 15 nt, Homo sapiens
  source 1..15: mol_type = transcribed RNA
gggtctggtgcggag

SEQ ID NO: 959 - RNA, 16 nt, Homo sapiens
  source 1..16: mol_type = transcribed RNA
ggacccagggagagac

SEQ ID NO: 960 - RNA, 25 nt, Homo sapiens
  source 1..25: mol_type = transcribed RNA
tgcggggctagggctaacagcagtc

SEQ ID NO: 961 - RNA, 15 nt, Homo sapiens
  source 1..15: mol_type = transcribed RNA
tgcggggctagggct

SEQ ID NO: 962 - RNA, 23 nt, Homo sapiens
  source 1..23: mol_type = transcribed RNA
tcggctctgggtctgtggggagc

SEQ ID NO: 963 - RNA, 15 nt, Homo sapiens
  source 1..15: mol_type = transcribed RNA
gcccggatacctcag

SEQ ID NO: 964 - RNA, 24 nt, Homo sapiens
  source 1..24: mol_type = transcribed RNA
ctgccctggcccgagggaccgact

SEQ ID NO: 965 - RNA, 15 nt, Homo sapiens
  source 1..15: mol_type = transcribed RNA
ctgccctggcccgag

SEQ ID NO: 966 - RNA, 25 nt, Homo sapiens
  source 1..25: mol_type = transcribed RNA
gggtggggatttgttgcattacttg

SEQ ID NO: 967 - RNA, 20 nt, Homo sapiens
  source 1..20: mol_type = transcribed RNA
gggtggggatttgttgcatt

SEQ ID NO: 968 - RNA, 26 nt, Homo sapiens
  source 1..26: mol_type = transcribed RNA
agggacgggacgcggtgcagtgttgt

SEQ ID NO: 969 - RNA, 15 nt, Homo sapiens
  source 1..15: mol_type = transcribed RNA
ggcgggcgggaggga

SEQ ID NO: 970 - RNA, 25 nt, Homo sapiens
  source 1..25: mol_type = transcribed RNA
aaggcagggcccccgctccccgggc

SEQ ID NO: 971 - RNA, 15 nt, Homo sapiens
  source 1..15: mol_type = transcribed RNA
gtgtgttgaggaagg

SEQ ID NO: 972 - RNA, 22 nt, Homo sapiens
  source 1..22: mol_type = transcribed RNA
cctcacacctgcctcgcccccc

SEQ ID NO: 973 - RNA, 15 nt, Homo sapiens
  source 1..15: mol_type = transcribed RNA
tcacacctgcctcgc

SEQ ID NO: 974 - RNA, 23 nt, Homo sapiens
  source 1..23: mol_type = transcribed RNA
gtgggggagaggctgtcttgtgt

SEQ ID NO: 975 - RNA, 15 nt, Homo sapiens
  source 1..15: mol_type = transcribed RNA
gtgtgggggagaggc

SEQ ID NO: 976 - RNA, 25 nt, Homo sapiens
  source 1..25: mol_type = transcribed RNA
actcggcgtggcgtcggtcgtggta

SEQ ID NO: 977 - RNA, 15 nt, Homo sapiens
  source 1..15: mol_type = transcribed RNA
actcggcgtggcgtc

SEQ ID NO: 978 - RNA, 27 nt, Homo sapiens
  source 1..27: mol_type = transcribed RNA
atcacattgccagggatttccaaccga

SEQ ID NO: 979 - RNA, 15 nt, Homo sapiens
  source 1..15: mol_type = transcribed RNA
aatcacattgccagg

SEQ ID NO: 980 - RNA, 26 nt, Homo sapiens
  source 1..26: mol_type = transcribed RNA
tagcaccatttgaaatcagtgttctt

SEQ ID NO: 981 - RNA, 15 nt, Homo sapiens
  source 1..15: mol_type = transcribed RNA
ctagcaccatttgaa

SEQ ID NO: 982 - RNA, 21 nt, Homo sapiens
  source 1..21: mol_type = transcribed RNA
cccaggctggagcgagtgcag

SEQ ID NO: 983 - RNA, 15 nt, Homo sapiens
  source 1..15: mol_type = transcribed RNA
agctcactgcagcct

SEQ ID NO: 984 - RNA, 21 nt, Homo sapiens
  source 1..21: mol_type = transcribed RNA
aagaaggcggtcggtctgcgg

SEQ ID NO: 985 - RNA, 20 nt, Homo sapiens
  source 1..20: mol_type = transcribed RNA
ccccggggagcccggcggtg

SEQ ID NO: 986 - RNA, 15 nt, Homo sapiens
  source 1..15: mol_type = transcribed RNA
accccggggagcccg

SEQ ID NO: 987 - RNA, 19 nt, Homo sapiens
  source 1..19: mol_type = transcribed RNA
gcaggcgaggctgggctga

SEQ ID NO: 988 - RNA, 16 nt, Homo sapiens
  source 1..16: mol_type = transcribed RNA
aggcgaggctgggctg

SEQ ID NO: 989 - RNA, 24 nt, Homo sapiens
  source 1..24: mol_type = transcribed RNA
gtgagtgggagccggtggggctgg

SEQ ID NO: 990 - RNA, 15 nt, Homo sapiens
  source 1..15: mol_type = transcribed RNA
ggggctggagtaagg

SEQ ID NO: 991 - RNA, 22 nt, Homo sapiens
  source 1..22: mol_type = transcribed RNA
ccccggtgttggggcgcgtctg

SEQ ID NO: 992 - RNA, 21 nt, Homo sapiens
  source 1..21: mol_type = transcribed RNA
cccggtgttggggcgcgtctg

SEQ ID NO: 993 - RNA, 20 nt, Homo sapiens
  source 1..20: mol_type = transcribed RNA
ggctggtcagatgggagtgg

SEQ ID NO: 994 - RNA, 26 nt, Homo sapiens
  source 1..26: mol_type = transcribed RNA
gactatagaactttccccctcatccc

SEQ ID NO: 995 - RNA, 15 nt, Homo sapiens
  source 1..15: mol_type = transcribed RNA
aactttccccctcat

SEQ ID NO: 996 - RNA, 25 nt, Homo sapiens
  source 1..25: mol_type = transcribed RNA
tgcaggcagaagtggggctgacagg

SEQ ID NO: 997 - RNA, 20 nt, Homo sapiens
  source 1..20: mol_type = transcribed RNA
ctgcaggcagaagtggggct

SEQ ID NO: 998 - RNA, 17 nt, Homo sapiens
  source 1..17: mol_type = transcribed RNA
tcctagtcacggcacca

SEQ ID NO: 999 - RNA, 28 nt, Homo sapiens
  source 1..28: mol_type = transcribed RNA
gtatggtattgcacttgtcccggcctgt

SEQ ID NO: 1000 - RNA, 15 nt, Homo sapiens
  source 1..15: mol_type = transcribed RNA
tattgcacttgtccc